(19) United States
(12) Patent Application Publication
(10) Pub. No.: US 2021/0261636 A1
(43) Pub. Date: Aug. 26, 2021

(54) NEOANTIGENS EXPRESSED IN MULTIPLE MYELOMA AND THEIR USES

(71) Applicant: Janssen Biotech, Inc., Horsham, PA (US)

(72) Inventors: Vipul BHARGAVA, Warrington, PA (US); Vinod Krishna, Philadelphia, PA (US); Jaymala Patel, Bensalem, PA (US); David J. Pocalyko, Doylestown, PA (US); Yashoda Rani Rajpurohit, Hillsborough, NJ (US); Pegah Safabakhsh, Jenkintown, PA (US); Manuel Alejandro Sepulveda, West Windsor, PA (US)

(21) Appl. No.: 17/174,590

(22) Filed: Feb. 12, 2021

Related U.S. Application Data
(60) Provisional application No. 62/976,386, filed on Feb. 14, 2020.

(51) Int. Cl. C07K 14/47; A61K 39/00; A61K 45/06; A61P 37/04

(52) U.S. Cl. CPC C07K 14/4748; A61K 39/0011; A61K 45/06; A61K 2039/804; A61K 2039/5256; A61K 2039/53; A61P 37/04

(57) ABSTRACT

The disclosure relates to multiple myeloma neoantigens, polynucleotides encoding them, vectors, host cells, recombinant virus particles, vaccines comprising the neoantigens, proteinaceous molecules binding the multiple myeloma neoantigens, and methods of making and using them.

### CROSS-REFERENCE TO RELATED APPLICATIONS

This application claims priority to U.S. Provisional Application No. 62/976,386 filed on Feb. 14, 2020, titled “NEOANTIGENS EXPRESSED IN MULTIPLE MYELOMA AND THEIR USES” which is incorporated by reference in its entirety.

### SEQUENCE LISTING

The instant application contains a Sequence Listing which has been submitted electronically in ASCII format and is hereby incorporated by reference in its entirety. The ASCII copy, created on Jan. 22, 2021, is named JBI6239USNP1_SL.txt and is 145,351 bytes in size.

### FIELD

The disclosure relates to multiple myeloma neoantigens, polynucleotides encoding them, vectors, host cells, vaccines comprising the neoantigens, proteinaceous molecules binding the multiple myeloma neoantigens, and methods of making and using them.

### BACKGROUND

Multiple myeloma causes significant morbidity and mortality. It accounts for approximately 1% of all malignancies and 13% of hematologic cancers worldwide. Approximately 50,000 patients per year are diagnosed with multiple myeloma in the EU and US, and 30,000 patients per year die due to multiple myeloma.

The majority of patients with multiple myeloma produce a monoclonal protein (paraprotein, M-protein or M-component) which is an immunoglobulin (Ig) or a fragment of one that has lost its function (Kyle and Rajkumar, Leukemia 23:3-9, 2009; Palumbo and Anderson, N Engl J Med 364:1046-1060, 2011). Normal immunoglobulin levels are compromised in patients, leading to susceptibility of infections. The proliferating multiple myeloma cells displace the normal bone marrow leading to dysfunction in normal hematopoietic tissue and destruction of the normal bone marrow architecture, which is reflected by clinical findings such as anemia, paraprotein in serum or urine, and bone resorption seen as diffuse osteoporosis or lytic lesions shown in radiographs (Kyle et al., Mayo Clin Proc 78:21-33, 2003). Furthermore, hypercalcemia, renal insufficiency or failure, and neurological complications are frequently seen. A small minority of patients with multiple myeloma are non-secretory.

Treatment choices for multiple myeloma vary with age, comorbidity, the aggressiveness of the disease, and related prognostic factors (Palumbo and Anderson, N Engl J Med 364:1046-1060, 2011). Newly diagnosed patients with multiple myeloma are typically categorized into 2 subpopulations usually defined by their age and suitability for the subsequent approach to treatment. Younger patients will typically receive an induction regimen followed by consolidation treatment with high-dose chemotherapy (HDC) and autologous stem cell transplantation (ASCT). For those not considered suitable for HDC and ASCT, longer-term treatment with multi-agent combinations including alkylators, high-dose steroids, and novel agents are currently considered as standards of care. In general, patients over the age of 65 or with significant comorbidities are usually not considered eligible for HDC and ASCT. For many years, the oral combination melphalan-prednisone (MP) was considered the standard of care for patients with multiple myeloma who were not eligible for ASCT (Gay and Palumbo, Blood Reviews 25:65-73, 2011). The advent of immunomodulatory agents (IMiDs) and proteasome inhibitors (PIs) has led to a multiplicity of new treatment options for newly diagnosed patients not considered suitable for transplant-based therapy.

Despite various attempts to improve the treatment of multiple myeloma, a need still remains for therapies against multiple myeloma.

### BRIEF SUMMARY

The disclosure provides an isolated polypeptide comprising an amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, or 421, or fragments thereof.

The disclosure also provides an isolated heterologous polypeptide comprising two or more polypeptides selected from the group consisting of SEQ ID NOs: 1, 3, 5, 7, 9, 11, 13, 15, 17, 19, 21, 23,25,27,29,31,33,35,37,39,41,43,45,47,49,51,53,55,57,59,61,63,65,67,69,71,73,75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, and 421, and fragments thereof.

The disclosure also provides an isolated polynucleotide comprising a sequence of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, or 422, or fragments thereof.

The disclosure also provides vectors comprising the polynucleotides encoding for the polypeptides disclosed herein.

The disclosure also provides viruses or recombinant viruses comprising the vectors of the disclosure.

The disclosure also provides cells comprising or transduced with the vectors of the disclosure or the recombinant viruses of the disclosure.

The disclosure also provides a vaccine comprising the polynucleotides of the disclosure.

The disclosure also provides a vaccine comprising the polypeptides of the disclosure.

The disclosure also provides a vaccine comprising the vectors of the disclosure.

The disclosure also provides a vaccine comprising recombinant viruses of the disclosure.

The disclosure also provides a vaccine comprising the self-replicating RNA molecule of the disclosure.

The disclosure also provides a method of preventing or treating a multiple myeloma in a subject, comprising administering to the subject a therapeutically effective amount of one or more vaccines of the disclosure, one or more virus or recombinant virus of the disclosure, or one or more pharmaceutical composition of the disclosure.

The disclosure also provides methods of inducing an immune response against one or more amino acid sequences of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213,215,217,219,221,223,225,227,229,231,233,235,237,239,241,243,245,247,249,251, 253,255,257,259,261,263,265,267,269,271,273,275,277,279,281,283,285,287,289,291, 293,295,297,299,301,303,305,307,309,311,313,315,317,319,321,323,325,327,329,331, 333,335,337,339,341,343,345,347,349,351,353,355,357,359,361,363,365,367,369,371, 373, 375, 377, 379, 381, 383, 385, 387, 389, 391,393, 395, 397, 399, 401, 403, 405, 407 or 421 in a subject, comprising administering to the subject one or more vaccines of the discolsure, or one or more recombinant viruses of the disclosure comprising the polynucleotides of the disclosure, and wherein the recombinant virus is Ad26, GAd20, MVA and/or admistering a self-replicating RNA molecule encoding polypeptides of the dislosure.

The disclosure also provides a method of treating or preventing a multiple myeloma in a subject, comprising

administering to the subject a therapeutically effective amount of a composition comprising a recombinant virus and/or a composition comprising a self-replicating RNA molecule encoding a heterologous polypeptide comprising two, three, four, five, six, seven, eight, nine, ten, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, or 204 polypeptides selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, and 421 and fragments thereof. In some embodiments, the recombinant virus is a Ad26, GAd20, or MVA virus. In some embodiements, In some embodiments, the administration comprises one or more administrations of the composition.

The disclosure also provides a method of treating or preventing a multiple myeloma in a subject, comprising administering to the subject

a first composition comprising a first heterologous polynucleotide encoding a first heterologous polypeptide, wherein the first heterologous polypeptide comprises two or more polypeptides selected from the group consisting of SEQ ID NOs: 1, 3, 5, 7, 9, 11, 13, 15, 17, 19, 21, 23,25,27,29,31,33,35,37,39,41,43,45,47,49,51,53,55,57,59,61,63,65,67,69,71,73,75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, and 421, and fragments thereof; and

a second composition comprising a second heterologous polynucleotide encoding a second heterologous polypeptide, wherein the second heterologous polypeptide comprises two or more polypeptides selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243,245,247,249,251,253,255,257,259,261,263,265,267,269,271,273,275,277,279,281, 283,285,287,289,291,293,295,297,299,301,303,305,307,309,311,313,315,317,319,321, 323,325,327,329,331,333,335,337,339,341,343,345,347,349,351,353,355,357,359,361, 363,365,367,369,371,373,375,377,379,381,383,385,387,389,391,393,395,397,399,401, 403, 405, 407, and 421, and fragments thereof; wherein the first heterologous polypeptide and the second heterologous polypeptide have distinct amino acid sequences.

The disclosure also provides a method of treating or preventing a multiple myeloma in a subject, comprising administering to the subject a therapeutically effective amount of a composition comprising a recombinant virus and/or a composition comprising a self-replicating RNA molecule encoding a heterologous polypeptide selected from SEQ ID NO: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, and 421

The disclosure also provides a method of inducing an immune response in a subject, comprising administering to the subject a compostion comprising a heterologous polynucleotide encoding a heterologous polypeptide, wherein the heterologous polypeptide comprises one or more polypeptides selected from the group consisting of SEQ ID NOs: 1, 3, 5, 7, 9, 11, 13, 15, 17, 19, 21, 23,25,27,29,31,33,35,37,39,41,43,45,47,49,51,53,55,57,59,61,63,65,67,69,71,73,75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203,205,207,209,211,213,215,217,219,221,223,225,227,229,231,233,235,237,239,241, 243,245,247,249,251,253,255,257,259,261,263,265,267,269,271,273,275,277,279,281, 283,285,287,289,291,293,295,297,299,301,303,305,307,309,311,313,315,317,319,321, 323,325,327,329,331,333,335,337,339,341,343,345,347,349,351,353,355,357,359,361, 363,365,367,369,371,373,375,377,379,381,383,385,387,389,391,393,395,397,399,401, 403, 405, 407, and 421 and fragments thereof; and wherein the administration comprises one or more administrations of the composition and wherein the heterologous polypeptides have distinct amino acid sequences.

The disclosure also a method of inducing an immune response in a subject, comprising administering to the subject a compostion comprising a heterologous polynucleotide encoding a heterologous polypeptide, wherein the heterologous polypeptide comprises one or more polypeptides selected from the group consisting of SEQ ID NO: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, and 421

The disclosure also provides an isolated proteinaceous molecule that specifically binds the polypeptide of the disclosure.

The disclosure also provides a method of preventing or treating a multiple myeloma in a subject, comprising administering to the subject the proteinaceous molecule of the disclosure.

The disclosure also provides administering an anti-CTLA-4 antibody, an anti-PD-1 or an anti-PD-L1 antibody in combination with any of the compositions comprising polynucleotides, polypeptides, vectors, or viruses disclosed herein.

It is to be understood, that the above embodiments of the invention encompass polypeptides comprising, in addition to the specifically recited polypeptides and fragments thereof, also additional polypeptide sequences, including one or more polypeptides different from those specifically recited. Similarly, the above embodiments of the invention also encompass polynucleotides comprising, in addition to the specifically recited polynucleotides and fragments thereof, also additional polynucleotide sequences, including one or more polynucleotides different from those specifically recited.

### BRIEF DESCRIPTION OF THE DRAWINGS

FIG. 1 shows a cartoon of chimeric read-through fusions between Gene A and Gene B. Neoantigenic peptide sequences arise at the breakpoint junction.

FIG. 2 shows a cartoon of gene fusions resulting from chromosomal alteration, such as DNA translocations.

FIG. 3 shows a cartoon of splice variants with alternative 5′ or 3′ splice sites, retained introns, excluded exons or alternative terminations or insertions.

FIG. 4 shows the cartoon for approach of identification of splice variants.

FIG. 5A, FIG. 5B, FIG. 5C and FIG. 5D shows a heat map representing tumor restricted expression of Multiple Myeloma (MM) neoantigens. These antigens do not have detectable expression in either healthy tissues or immune cells derived from healthy donors. The immune cell types (first 15 rows) were derived from three healthy donors (donor ID: D001003103, D001000682 and D001004622). CD138+ MM samples are labeled with prefix ‘MM’. The raw Ct values were normalized against the expression of an endogenous control gene, RPL13A. The black cells represent high expression (ΔCt below 15) in each sample. Four neoantigens (FUST, FUS20, AS43 and AS76) showing expression in normal donor-derived B-cells and plasma cells were retained in this group.

FIG. 6A, FIG. 6B, FIG. 6C and FIG. 6D shows a heat map representing Multiple Myeloma neoantigen candidates with expression in both control (tissues and immune cells derived from healthy donors) and tumor samples. The immune cell types (first 15 rows) were derived from three healthy donors (donor ID: D001003103, D001000682 and D001004622). CD138+ MM samples are labeled with prefix ‘MM’. The raw Ct values were normalized against the expression of an endogenous control gene, RPL13A. The black cells represent high expression (ΔCt below 15) in each sample.

FIG. 7A and FIG. 7B shows a representative dot plots depicting positive immunogenic responses of neoantigens by using exogenous autologous healthy donor restimulation assay. Immunogenicity responses were measured by estimating TNFα IFNγ double positive cells in the CD4+ and/or CD8+ T-cell populations. A response is considered positive if TNFα IFNγ double positive fraction was greater than or equal to three-fold over unstimulated cells (DMSO negative control) with a minimum frequency >=0.01%.

FIG. 8A and FIG. 8B show the number of donors with positive immunogenicity responses (CD8+ and/or CD4+ T-cell) for gene fusion associated neoantigens. FIG. 9A and FIG. 9B show the number of donors with positive immunogenicity responses (CD8+ and/or CD4+ T cells) for alternative splicing associated neoantigens.

### DETAILED DESCRIPTION

### Definitions

All publications, including but not limited to patents and patent applications, cited in this specification are herein incorporated by reference as though fully set forth.

It is to be understood that the terminology used herein is for describing particular embodiments only and is not intended to be limiting. Unless defined otherwise, all technical and scientific terms used herein have the same meaning as commonly understood by one of ordinary skill in the art to which the disclosure pertains.

Although any methods and materials similar or equivalent to those described herein may be used in the practice for testing of the present disclosure, exemplary materials and methods are described herein. In describing and claiming the present disclosure, the following terminology will be used.

As used in this specification and the appended claims, the singular forms “a,” “an,” and “the” include plural referents unless the content clearly dictates otherwise. Thus, for example, reference to “a cell” includes a combination of two or more cells, and the like.

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed disclosure. Embodiments described in terms of the phrase “comprising” (or its equivalents) also provide as embodiments those independently described in terms of “consisting of” and “consisting essentially of”.

As used in this specification and the appended claims, the phrase “and fragments thereof” when appended to a list includes all members of the associated list. The list may comprise a Markush group so that, as an example, the phrase “the group consisting of peptides A, B, and C, and fragments thereof” specifies or recites a Markush group including A, B, C, fragments of A, fragments of B, and fragments of C.

“Isolated” refers to a homogenous population of molecules (such as synthetic polynucleotides or polypeptides) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Polynucleotide” refers to a synthetic molecule comprising a chain of nucleotides covalently linked by a sugar-phosphate backbone or other equivalent covalent chemistry. cDNA is a typical example of a polynucleotide.

“Polypeptide” or “protein” refers to a molecule that comprises at least two amino acid residues linked by a peptide bond to form a polypeptide. Small polypeptides of less than 50 amino acids may be referred to as “peptides”.

“Immunogenic fragment” refers to a polypeptide that is recognized by cytotoxic T lymphocytes, helper T lymphocytes or B cells when the fragment is in complex with MHC class I or MHC class II molecules.

“In-frame” refers to the reading frame of codons in a first polynucleotide being the same as the reading frame of codons in a second polynucleotide which are joined together to form a heterologous polynucleotide. In-frame heterologous polynucleotide encodes a heterologous polypeptide encoded by both the first polynucleotide and the second polynucleotide.

“Immunogenic” refers to a polypeptide that comprises one or more immunogenic fragments.

“Heterologous” refers to two or more polynucleotides or two or more polypeptides that are not found in the same relationship to each other in nature.

“Heterologous polynucleotide” refers to a non-naturally occurring polynucleotide that encodes two or more neoantigens as described herein.

“Heterologous polypeptide” refers to a non-naturally occurring polypeptide comprising two or more neoantigen polypeptides as described herein.

“Non-naturally occurring” refers to a molecule that does not exist in nature.

“Vector” refers to a polynucleotide capable of being duplicated within a biological system or that can be moved between such systems. Vector polynucleotides typically contain elements, such as origins of replication, polyadenylation signal or selection markers, that function to facilitate the duplication or maintenance of these polynucleotides in a biological system. Examples of such biological systems may include a cell, virus, animal, plant, and reconstituted biological systems utilizing biological components capable of duplicating a vector. The polynucleotide comprising a vector may be DNA or RNA molecules or a hybrid of these.

“Expression vector” refers to a vector that can be utilized in a biological system or in a reconstituted biological system to direct the translation of a polypeptide encoded by a polynucleotide sequence present in the expression vector.

“Viral vector” refers to a vector construct that includes at least one polynucleotide element of viral origin and has the capacity to be packaged into a viral vector particle.

“Neoantigen” refers to a polypeptide that is present in CD138⁺ cells isolated from bone marrow aspirates from multiple myeloma patients that has at least one alteration that makes it distinct from the corresponding wild-type polypeptide present in non-malignant tissue, e.g., via mutation in a tumor cell or post-translational modification specific to a tumor cell. A mutation can include a frameshift or nonframeshift insertion or deletion, missense or nonsense substitution, splice site alteration, aberrant splice variants, genomic rearrangement or gene fusion, or any genomic or expression alteration giving rise to the neoantigen.

“Prevalence” refers to a percentage of a population studied harboring a multiple myeloma neoantigen.

“Recombinant” refers to polynucleotides, polypeptides, vectors, viruses and other macromolecules that are prepared, expressed, created or isolated by recombinant means.

“Vaccine” refers to a composition that comprises one or more immunogenic polypeptides, immunogenic polynucleotides or fragments, or any combination thereof intentionally administered to induce acquired immunity in the recipient (e.g. subject).

“Treat”, “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Prevent”, “preventing”, “prevention”, or “prophylaxis” of a disease or disorder means preventing that a disorder occurs in subject.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

“Relapsed” refers to the return of a disease or the signs and symptoms of a disease after a period of improvement after prior treatment with a therapeutic.

“Refractory” refers to a disease that does not respond to a treatment. A refractory disease can be resistant to a treatment before or at the beginning of the treatment, or a refractory disease can become resistant during a treatment.

“Replicon” refers to a viral nucleic acid that is capable of directing the generation of copies of itself and includes RNA as well as DNA. For example, double-stranded DNA versions of arterivirus genomes can be used to generate a single-stranded RNA transcript that constitutes an arterivirus replicon. Generally, a viral replicon contains the complete genome of the virus. “Sub-genomic replicon” refers to a viral nucleic acid that contains something less than the full complement of genes and other features of the viral genome yet is still capable of directing the generation of copies of itself. For example, the sub-genomic replicons of arterivirus may contain most of the genes for the non-structural proteins of the virus but are missing most of the genes coding for the structural proteins. Sub-genomic replicons are capable of directing the expression of all of the viral genes necessary for the replication of the viral sub-genome (replication of the sub-genomic replicon), without the production of viral particles.

“RNA replicon” (or “self-replicating RNA molecule”) refer to RNA which contains all of the genetic information required for directing its own amplification or self-replication within a permissive cell. To direct its own replication, the RNA molecule 1) encodes polymerase, replicase, or other proteins which may interact with viral or host cell-derived proteins, nucleic acids or ribonucleoproteins to catalyze the RNA amplification process; and 2) contain cis-acting RNA sequences required for replication and transcription of the replicon-encoded RNA. Self-replicating RNA is typically derived from the genomes of positive strand RNA viruses and can be used as basis of introducing foreign sequences to host cells by replacing viral sequences encoding structural or non-structural genes or inserting the foreign sequences 5′ or 3′ of the sequences encoding the structural or non-structural genes. Foreign sequences may also be introduced into the subgenomic regions of alphaviruses. Self-replicating RNA may be packaged into recombinant virus particles, such as recombinant alphavirus particles or alternatively delivered to the host using lipid nanoparticles (LNP). Self-replicating RNA may be at least 1 kb or at least 2 kb or at least 3 kb or at least 4 kb or at least 5 kb or at least 6 kb or at least 7 kb or at least 8 kb or at least 10 kb or at least 12 kb or at least 15 kb or at least 17 kb or at least 19 kb or at least 20 kb in size, or can be 100 bp-8 kb or 500 bp-8 kb or 500 bp-7 kb or 1-7 kb or 1-8 kb or 2-15 kb or 2-20 kb or 5-15 kb or 5-20 kb or 7-15 kb or 7-18 kb or 7-20 kb in size. Self-replicating RNAs are described, for example, in WO2017/180770, WO2018/075235, WO2019143949A2,

“Newly diagnosed” refers to a human subject who has been diagnosed with but has not yet received treatment for a disease, such as multiple myeloma.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

“In combination with” means that two or more therapeutic agents are be administered to a subject together in a mixture, concurrently as single agents or sequentially as single agents in any order.

“Enhance” or “induce” when in reference to an immune response refers to increasing the scale and/or efficiency of an immune response or extending the duration of the immune response. The terms are used interchangeably with “augment”.

“Immune response” refers to any response to an immunogenic polypeptide or polynucleotide or fragment by the immune system of a vertebrate subject. Exemplary immune responses include local and systemic cellular as well as humoral immunity, such as cytotoxic T lymphocyte (CTL) responses, including antigen-specific induction of CD8⁺ CTLs, helper T-cell responses including T-cell proliferative responses and cytokine release, and B-cell responses including antibody response.

“Specifically binds”, “specific binding”, “specifically binding” or “binds” refer to a proteinaceous molecule binding to an antigen or an epitope within the antigen (e.g. to multiple myeloma neoantigen) with greater affinity than for other antigens. Typically, the proteinaceous molecule binds to the antigen or the epitope within the antigen with an equilibrium dissociation constant (KD) of about 1×10⁻⁷M or less, for example about 5×10⁻⁸M or less, about 1×10⁻⁸M or less, about 1×10⁻⁹M or less, about 1×10⁻¹⁰ M or less, about 1×10⁻¹¹ M or less, or about 1×10¹² M or less, typically with the KD that is at least one hundred fold less than its KD for binding to a non-specific antigen (e.g., BSA, casein). In the context of the multiple myeloma neoantigens described here, “specific binding” refers to binding of the proteinaceous molecule to the multiple myeloma neoantigen without detectable binding to a wild-type protein the neoantigen is a variant of.

“Variant”, “mutant” or “altered” refers to a polypeptide or a polynucleotide that differs from a reference polypeptide or a reference polynucleotide by one or more modifications, for example one or more substitutions, insertions or deletions.

“Antibody” refers to an immunoglobulin molecule including monoclonal antibodies including murine, human, humanized and chimeric monoclonal antibodies, antigen-binding fragments, bispecific or multispecific antibodies, dimeric, tetrameric or multimeric antibodies, single chain antibodies, domain antibodies and any other modified configuration of the immunoglobulin molecule that comprises an antigen binding site of the required specificity.

“Alternative scaffold” refers to a single chain protein framework that contains a structured core associated with variable domains of high conformational tolerance. The variable domains tolerate variation to be introduced without compromising scaffold integrity, and hence the variable domains can be engineered and selected for binding to a specific antigen.

“Chimeric antigen receptor” or “CAR” refers to engineered T cell receptors which graft a ligand or antigen specificity onto T cells (for example naïve T cells central memory T cells effector memory T cells or combinations thereof). CARS are also known as artificial T-cell receptors, chimeric T-cell receptors or chimeric immunoreceptors. CARs comprise an extracellular domain capable of binding to an antigen, a transmembrane domain and at least one intracellular domain. CAR intracellular domain comprises a polypeptide known to function as a domain that transmits a signal to cause activation or inhibition of a biological process in a cell. The transmembrane domain comprises any peptide or polypeptide known to span the cell membrane and that can function to link the extracellular and signaling domains. A chimeric antigen receptor may optionally comprise a hinge domain which serves as a linker between the extracellular and transmembrane domains.

“T cell receptor” or “TCR” refers to a molecule capable of recognizing a peptide when presented by an MHC molecule. Naturally occurring TCR heterodimer consists of an alpha (α) and beta (β) chain in around 95% of T-cells, whereas around 5% of T-cells have TCRs consisting of gamma (γ) and delta (δ) chains. Each chain of a natural TCR is a member of the immunoglobulin superfamily and possesses one N-terminal immunoglobulin (Ig)-variable (V) domain, one Ig-constant (C) domain, a transmembrane/cell membrane-spanning region, and a short cytoplasmic tail at the C-terminal end. The variable domain of both the TCR α chain and β chain have three hypervariable or complementarity determining regions (CDRs), CDR1, CDR2 and CDR3, which are responsible for recognizing processed antigens presented on MHC.

TCR may be a full length α/β or γ/δ heterodimer or a soluble molecule comprising a portion of the extracellular domain of the TCR that retains binding the peptide/MHC complex. TCR may be engineered into a single chain TCR.

“T cell receptor complex” or “TCR complex” refers to a known TCR complex comprising of a TCRα and TCRβ chains, CD3ε, CD3γ, CD3δ and CD3ζ molecules. In some instances, TCRα and TCRβ chains are replaced by TCRγ and TCRδ chains. The amino acid sequences of the various proteins forming the TCR complex are well-known.

“T cell” and “T lymphocyte” are interchangeable and used synonymously herein. T cell includes thymocytes, naïve T lymphocytes, memory T cells, immature T lymphocytes, mature T lymphocytes, resting T lymphocytes, or activated T lymphocytes. A T cell can be a T helper (Th) cell, for example a T helper 1 (Thl) or a T helper 2 (Th2) cell. The T cell can be a helper T cell (HTL; CD4⁺ T cell) CD4⁺ T cell, a cytotoxic T cell (CTL; CD8⁺ T cell), a tumor infiltrating cytotoxic T cell (TIL; CD8⁺ T cell), CD4⁺CD8⁺ T cell, or any other subset of T cells. Also included are “NKT cells”, which refer to a specialized population of T cells that express a semi-invariant αβ T-cell receptor, but also express a variety of molecular markers that are typically associated with NK cells, such as NK1.1. NKT cells include NK1.1⁺ and NK1.1⁻, as well as CD4⁺, CD4⁻, CD8⁺ and CD8⁻ cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain . γδ T cells can play a role in immunosurveillance and immunoregulation, and were found to be an important source of IL-17 and to induce robust CD8⁺ cytotoxic T cell response. Also included are “regulatory T cells” or “Tregs” which refer to T cells that suppress an abnormal or excessive immune response and play a role in immune tolerance. Tregs are typically transcription factor Foxp3-positive CD4⁺T cells and can also include transcription factor Foxp3-negative regulatory T cells that are IL-10-producing CD4⁺T cells.

“Natural killer cell” or “NK cell” refers to a differentiated lymphocyte with a CD 16+ CD56+ and/or CD57+ TCR-phenotype. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Antigen presenting cell” (APC) refers to any cell that presents on its surface an antigen in association with a major histocompatibility complex molecule, either MHC class I or MHC class II molecule, or both.

“Prime-boost” or “prime-boost regimen” refers to a method of treating a subject involving priming a T-cell response with a first vaccine followed by boosting the immune response with a second vaccine. The first vaccine and the second vaccine are typically distinct. These prime-boost immunizations elicit immune responses of greater height and breadth than can be achieved by priming and boosting with the same vaccine. The priming step initiates memory cells and the boost step expands the memory response. Boosting can occur once or multiple times.

“Facilitator element” refers to any polynucleotide or polypeptide element that is operably linked to a polynucleotide or a polypeptide, and include promoters, enhancers, polyadenylation signals, stop codons, protein tags, such as histidine tag, and the like. Facilitator elements herein include regulatory elements.

“Distinct” in the context of polypeptide or polynucleotide sequences refers to polypeptide or polynucleotide sequences that are not identical.

### Compositions of Matter

The disclosure relates to multiple myeloma neoantigens, polynucleotides encoding them, vectors, host cells, vaccines comprising the neoantigens or polynucleotides encoding the neoantigens, proteinaceous molecules binding the multiple myeloma neoantigens, and methods of making and using them. The disclosure also provides vaccines comprising the multiple myeloma neoantigens of the disclosure that are prevalent in a population of multiple myeloma patients, thereby providing a pan-vaccine that may be useful to treating a broad population of patients having diagnosed with various stages of multiple myeloma, such as smoldering multiple myeloma or advanced multiple myeloma.

Cancer cells produce neoantigens that result from genomic alterations and aberrant transcriptional programs. Neoantigen burden in patients has been associated with response to immunotherapy (Snyder et al., N Engl J Med. 2014 Dec 4; 371(23):2189-2199; Le et al., N Engl J Med. 2015 Jun. 25; 372(26):2509-20; Rizvi et al., Science. 2015 Apr. 3; 348(6230):124-8; Van Allen et al., Science. 2015 Oct. 9; 350(6257):207-211). The disclosure is based, at least in part, on the identification of multiple myeloma neoantigens that are common in multiple myeloma patients and hence can be utilized to develop a therapy amenable to treatment of a spectrum of multiple myeloma patients. One or more neoantigens or polynucleotides encoding the neoantigens of the disclosure may also be used for diagnostic or prognostic purposes.

### Polypeptides

Disclosed herein are polypeptides comprising multiple myeloma neoantigen sequences that may elicit an immune response in a subject.

The disclosure provides an isolated polypeptide comprising an amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, or 421, or fragments thereof. In some embodiments, the polypeptide is encoded by a polynucleotide sequence of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, or 422, or fragments thereof.

In some embodiments, the isolated polypeptide may comprise at least two or more multiple myeloma neoantigen sequences.

The disclosure also provides an isolated heterologous polypeptide comprising two or more polypeptides selected from the group consisting of SEQ ID NOs: 1, 3, 5, 7, 9, 11, 13, 15, 17, 19, 21, 23,25,27,29,31,33,35,37,39,41,43,45,47,49,51,53,55,57,59,61,63,65,67,69,71,73,75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, and 421, and fragments thereof. In some embodiments, the two or more polypeptides disclosed herein may be present in tandem repeats in any order.

The disclosure also provides an isolated polypeptide comprising an amino acid sequence of SEQ ID NO: 1 or fragments thereof.

The disclosure also provides an isolated polypeptide comprising an amino acid sequence of SEQ ID NO: 3 or fragments thereof.

The disclosure also provides an isolated polypeptide comprising an amino acid sequence of SEQ ID NO: 5 or fragments thereof.

The disclosure also provides an isolated polypeptide comprising an amino acid sequence of SEQ ID NO: 7 or fragments thereof.

The disclosure also provides an isolated polypeptide comprising an amino acid sequence of SEQ ID NO: 9 or fragments thereof.

The disclosure also provides an isolated polypeptide comprising an amino acid sequence of SEQ ID NO: 11 or fragments thereof.

The disclosure also provides an isolated polypeptide comprising an amino acid sequence of SEQ ID NO: 13 or fragments thereof.

The disclosure also provides an isolated polypeptide comprising an amino acid sequence of SEQ ID NO: 15 or fragments thereof.

The disclosure also provides an isolated polypeptide comprising an amino acid sequence of SEQ ID NO: 17 or fragments thereof.

The disclosure also provides an isolated polypeptide comprising an amino acid sequence of SEQ ID NO: 19 or fragments thereof.

The disclosure also provides an isolated polypeptide comprising an amino acid sequence of SEQ ID NO: 21 or fragments thereof.

The disclosure also provides an isolated polypeptide comprising an amino acid sequence of SEQ ID NO: 23 or fragments thereof.

The disclosure also provides an isolated polypeptide comprising an amino acid sequence of SEQ ID NO: 25 or fragments thereof.

The disclosure also provides an isolated polypeptide comprising an amino acid sequence of SEQ ID NO: 27 or fragments thereof.

The disclosure also provides an isolated polypeptide comprising an amino acid sequence of SEQ ID NO: 29 or fragments thereof.

The disclosure also provides an isolated polypeptide comprising an amino acid sequence of SEQ ID NO: 31 or fragments thereof.

The disclosure also provides an isolated polypeptide comprising an amino acid sequence of SEQ ID NO: 33 or fragments thereof.

The disclosure also provides an isolated polypeptide comprising an amino acid sequence of SEQ ID NO: 35 or fragments thereof.

The disclosure also provides an isolated polypeptide comprising an amino acid sequence of SEQ ID NO: 37 or fragments thereof.

The disclosure also provides an isolated polypeptide comprising an amino acid sequence of SEQ ID NO: 39 or fragments thereof.

The disclosure also provides an isolated polypeptide comprising an amino acid sequence of SEQ ID NO: 41 or fragments thereof.

The disclosure also provides an isolated polypeptide comprising an amino acid sequence of SEQ ID NO: 43 or fragments thereof.

The disclosure also provides an isolated polypeptide comprising an amino acid sequence of SEQ ID NO: 45 or fragments thereof.

The disclosure also provides an isolated polypeptide comprising an amino acid sequence of SEQ ID NO: 47 or fragments thereof.

The disclosure also provides an isolated polypeptide comprising an amino acid sequence of SEQ ID NO: 49 or fragments thereof.

The disclosure also provides an isolated polypeptide comprising an amino acid sequence of SEQ ID NO: 51 or fragments thereof.

The disclosure also provides an isolated polypeptide comprising an amino acid sequence of SEQ ID NO: 53 or fragments thereof.

The disclosure also provides an isolated polypeptide comprising an amino acid sequence of SEQ ID NO: 55 or fragments thereof.

The disclosure also provides an isolated polypeptide comprising an amino acid sequence of SEQ ID NO: 57 or fragments thereof.

The disclosure also provides an isolated polypeptide comprising an amino acid sequence of SEQ ID NO: 59 or fragments thereof.

The disclosure also provides an isolated polypeptide comprising an amino acid sequence of SEQ ID NO: 61 or fragments thereof.

The disclosure also provides an isolated polypeptide comprising an amino acid sequence of SEQ ID NO: 63 or fragments thereof.

The disclosure also provides an isolated polypeptide comprising an amino acid sequence of SEQ ID NO: 65 or fragments thereof.

The disclosure also provides an isolated polypeptide comprising an amino acid sequence of SEQ ID NO: 67 or fragments thereof.

The disclosure also provides an isolated polypeptide comprising an amino acid sequence of SEQ ID NO: 69 or fragments thereof.

The disclosure also provides an isolated polypeptide comprising an amino acid sequence of SEQ ID NO: 71 or fragments thereof.

The disclosure also provides an isolated polypeptide comprising an amino acid sequence of SEQ ID NO: 73 or fragments thereof.

The disclosure also provides an isolated polypeptide comprising an amino acid sequence of SEQ ID NO: 75 or fragments thereof.

The disclosure also provides an isolated polypeptide comprising an amino acid sequence of SEQ ID NO: 77 or fragments thereof.

The disclosure also provides an isolated polypeptide comprising an amino acid sequence of SEQ ID NO: 79 or fragments thereof.

The disclosure also provides an isolated polypeptide comprising an amino acid sequence of SEQ ID NO: 81 or fragments thereof.

The disclosure also provides an isolated polypeptide comprising an amino acid sequence of SEQ ID NO: 83 or fragments thereof.

The disclosure also provides an isolated polypeptide comprising an amino acid sequence of SEQ ID NO: 85 or fragments thereof.

The disclosure also provides an isolated polypeptide comprising an amino acid sequence of SEQ ID NO: 87 or fragments thereof.

The disclosure also provides an isolated polypeptide comprising an amino acid sequence of SEQ ID NO: 89 or fragments thereof.

The disclosure also provides an isolated polypeptide comprising an amino acid sequence of SEQ ID NO: 91 or fragments thereof.

The disclosure also provides an isolated polypeptide comprising an amino acid sequence of SEQ ID NO: 93 or fragments thereof.

The disclosure also provides an isolated polypeptide comprising an amino acid sequence of SEQ ID NO: 95 or fragments thereof.

The disclosure also provides an isolated polypeptide comprising an amino acid sequence of SEQ ID NO: 97 or fragments thereof.

The disclosure also provides an isolated polypeptide comprising an amino acid sequence of SEQ ID NO: 99 or fragments thereof.

The disclosure also provides an isolated polypeptide comprising an amino acid sequence of SEQ ID NO: 101 or fragments thereof.

The disclosure also provides an isolated polypeptide comprising an amino acid sequence of SEQ ID NO: 103 or fragments thereof.

The disclosure also provides an isolated polypeptide comprising an amino acid sequence of SEQ ID NO: 105 or fragments thereof.

The disclosure also provides an isolated polypeptide comprising an amino acid sequence of SEQ ID NO: 107 or fragments thereof.

The disclosure also provides an isolated polypeptide comprising an amino acid sequence of SEQ ID NO: 109 or fragments thereof.

The disclosure also provides an isolated polypeptide comprising an amino acid sequence of SEQ ID NO: 111 or fragments thereof.

The disclosure also provides an isolated polypeptide comprising an amino acid sequence of SEQ ID NO: 113 or fragments thereof.

The disclosure also provides an isolated polypeptide comprising an amino acid sequence of SEQ ID NO: 115 or fragments thereof.

The disclosure also provides an isolated polypeptide comprising an amino acid sequence of SEQ ID NO: 117 or fragments thereof.

The disclosure also provides an isolated polypeptide comprising an amino acid sequence of SEQ ID NO: 119 or fragments thereof.

The disclosure also provides an isolated polypeptide comprising an amino acid sequence of SEQ ID NO: 121 or fragments thereof.

The disclosure also provides an isolated polypeptide comprising an amino acid sequence of SEQ ID NO: 123 or fragments thereof.

The disclosure also provides an isolated polypeptide comprising an amino acid sequence of SEQ ID NO: 125 or fragments thereof.

The disclosure also provides an isolated polypeptide comprising an amino acid sequence of SEQ ID NO: 127 or fragments thereof.

The disclosure also provides an isolated polypeptide comprising an amino acid sequence of SEQ ID NO: 129 or fragments thereof.

The disclosure also provides an isolated polypeptide comprising an amino acid sequence of SEQ ID NO: 131 or fragments thereof.

The disclosure also provides an isolated polypeptide comprising an amino acid sequence of SEQ ID NO: 133 or fragments thereof.

The disclosure also provides an isolated polypeptide comprising an amino acid sequence of SEQ ID NO: 135 or fragments thereof.

The disclosure also provides an isolated polypeptide comprising an amino acid sequence of SEQ ID NO: 137 or fragments thereof.

The disclosure also provides an isolated polypeptide comprising an amino acid sequence of SEQ ID NO: 139 or fragments thereof.

The disclosure also provides an isolated polypeptide comprising an amino acid sequence of SEQ ID NO: 141 or fragments thereof.

The disclosure also provides an isolated polypeptide comprising an amino acid sequence of SEQ ID NO: 143 or fragments thereof.

The disclosure also provides an isolated polypeptide comprising an amino acid sequence of SEQ ID NO: 145 or fragments thereof.

The disclosure also provides an isolated polypeptide comprising an amino acid sequence of SEQ ID NO: 147 or fragments thereof.

The disclosure also provides an isolated polypeptide comprising an amino acid sequence of SEQ ID NO: 149 or fragments thereof.

The disclosure also provides an isolated polypeptide comprising an amino acid sequence of SEQ ID NO: 151 or fragments thereof.

The disclosure also provides an isolated polypeptide comprising an amino acid sequence of SEQ ID NO: 153 or fragments thereof.

The disclosure also provides an isolated polypeptide comprising an amino acid sequence of SEQ ID NO: 155 or fragments thereof.

The disclosure also provides an isolated polypeptide comprising an amino acid sequence of SEQ ID NO: 157 or fragments thereof.

The disclosure also provides an isolated polypeptide comprising an amino acid sequence of SEQ ID NO: 159 or fragments thereof.

The disclosure also provides an isolated polypeptide comprising an amino acid sequence of SEQ ID NO: 161 or fragments thereof.

The disclosure also provides an isolated polypeptide comprising an amino acid sequence of SEQ ID NO: 163 or fragments thereof.

The disclosure also provides an isolated polypeptide comprising an amino acid sequence of SEQ ID NO: 165 or fragments thereof.

The disclosure also provides an isolated polypeptide comprising an amino acid sequence of SEQ ID NO: 167 or fragments thereof.

The disclosure also provides an isolated polypeptide comprising an amino acid sequence of SEQ ID NO: 169 or fragments thereof.

The disclosure also provides an isolated polypeptide comprising an amino acid sequence of SEQ ID NO: 171 or fragments thereof.

The disclosure also provides an isolated polypeptide comprising an amino acid sequence of SEQ ID NO: 173 or fragments thereof.

The disclosure also provides an isolated polypeptide comprising an amino acid sequence of SEQ ID NO: 175 or fragments thereof.

The disclosure also provides an isolated polypeptide comprising an amino acid sequence of SEQ ID NO: 177 or fragments thereof.

The disclosure also provides an isolated polypeptide comprising an amino acid sequence of SEQ ID NO: 179 or fragments thereof.

The disclosure also provides an isolated polypeptide comprising an amino acid sequence of SEQ ID NO: 181 or fragments thereof.

The disclosure also provides an isolated polypeptide comprising an amino acid sequence of SEQ ID NO: 183 or fragments thereof.

The disclosure also provides an isolated polypeptide comprising an amino acid sequence of SEQ ID NO: 185 or fragments thereof.

The disclosure also provides an isolated polypeptide comprising an amino acid sequence of SEQ ID NO: 187 or fragments thereof.

The disclosure also provides an isolated polypeptide comprising an amino acid sequence of SEQ ID NO: 189 or fragments thereof.

The disclosure also provides an isolated polypeptide comprising an amino acid sequence of SEQ ID NO: 191 or fragments thereof.

The disclosure also provides an isolated polypeptide comprising an amino acid sequence of SEQ ID NO: 193 or fragments thereof.

The disclosure also provides an isolated polypeptide comprising an amino acid sequence of SEQ ID NO: 195 or fragments thereof.

The disclosure also provides an isolated polypeptide comprising an amino acid sequence of SEQ ID NO: 197 or fragments thereof.

The disclosure also provides an isolated polypeptide comprising an amino acid sequence of SEQ ID NO: 199 or fragments thereof.

The disclosure also provides an isolated polypeptide comprising an amino acid sequence of SEQ ID NO: 201 or fragments thereof.

The disclosure also provides an isolated polypeptide comprising an amino acid sequence of SEQ ID NO: 203 or fragments thereof.

The disclosure also provides an isolated polypeptide comprising an amino acid sequence of SEQ ID NO: 205 or fragments thereof.

The disclosure also provides an isolated polypeptide comprising an amino acid sequence of SEQ ID NO: 207 or fragments thereof.

The disclosure also provides an isolated polypeptide comprising an amino acid sequence of SEQ ID NO: 209 or fragments thereof.

The disclosure also provides an isolated polypeptide comprising an amino acid sequence of SEQ ID NO: 211 or fragments thereof.

The disclosure also provides an isolated polypeptide comprising an amino acid sequence of SEQ ID NO: 213 or fragments thereof.

The disclosure also provides an isolated polypeptide comprising an amino acid sequence of SEQ ID NO: 215 or fragments thereof.

The disclosure also provides an isolated polypeptide comprising an amino acid sequence of SEQ ID NO: 217 or fragments thereof.

The disclosure also provides an isolated polypeptide comprising an amino acid sequence of SEQ ID NO: 219 or fragments thereof.

The disclosure also provides an isolated polypeptide comprising an amino acid sequence of SEQ ID NO: 221 or fragments thereof.

The disclosure also provides an isolated polypeptide comprising an amino acid sequence of SEQ ID NO: 223 or fragments thereof.

The disclosure also provides an isolated polypeptide comprising an amino acid sequence of SEQ ID NO: 225 or fragments thereof.

The disclosure also provides an isolated polypeptide comprising an amino acid sequence of SEQ ID NO: 227 or fragments thereof.

The disclosure also provides an isolated polypeptide comprising an amino acid sequence of SEQ ID NO: 229 or fragments thereof.

The disclosure also provides an isolated polypeptide comprising an amino acid sequence of SEQ ID NO: 231 or fragments thereof.

The disclosure also provides an isolated polypeptide comprising an amino acid sequence of SEQ ID NO: 233 or fragments thereof.

The disclosure also provides an isolated polypeptide comprising an amino acid sequence of SEQ ID NO: 235 or fragments thereof.

The disclosure also provides an isolated polypeptide comprising an amino acid sequence of SEQ ID NO: 237 or fragments thereof.

The disclosure also provides an isolated polypeptide comprising an amino acid sequence of SEQ ID NO: 239 or fragments thereof.

The disclosure also provides an isolated polypeptide comprising an amino acid sequence of SEQ ID NO: 241 or fragments thereof.

The disclosure also provides an isolated polypeptide comprising an amino acid sequence of SEQ ID NO: 243 or fragments thereof.

The disclosure also provides an isolated polypeptide comprising an amino acid sequence of SEQ ID NO: 245 or fragments thereof.

The disclosure also provides an isolated polypeptide comprising an amino acid sequence of SEQ ID NO: 247 or fragments thereof.

The disclosure also provides an isolated polypeptide comprising an amino acid sequence of SEQ ID NO: 249 or fragments thereof.

The disclosure also provides an isolated polypeptide comprising an amino acid sequence of SEQ ID NO: 251 or fragments thereof.

The disclosure also provides an isolated polypeptide comprising an amino acid sequence of SEQ ID NO: 253 or fragments thereof.

The disclosure also provides an isolated polypeptide comprising an amino acid sequence of SEQ ID NO: 255 or fragments thereof.

The disclosure also provides an isolated polypeptide comprising an amino acid sequence of SEQ ID NO: 257 or fragments thereof.

The disclosure also provides an isolated polypeptide comprising an amino acid sequence of SEQ ID NO: 259 or fragments thereof.

The disclosure also provides an isolated polypeptide comprising an amino acid sequence of SEQ ID NO: 261 or fragments thereof.

The disclosure also provides an isolated polypeptide comprising an amino acid sequence of SEQ ID NO: 263 or fragments thereof.

The disclosure also provides an isolated polypeptide comprising an amino acid sequence of SEQ ID NO: 265 or fragments thereof.

The disclosure also provides an isolated polypeptide comprising an amino acid sequence of SEQ ID NO: 267 or fragments thereof.

The disclosure also provides an isolated polypeptide comprising an amino acid sequence of SEQ ID NO: 269 or fragments thereof.

The disclosure also provides an isolated polypeptide comprising an amino acid sequence of SEQ ID NO: 271 or fragments thereof.

The disclosure also provides an isolated polypeptide comprising an amino acid sequence of SEQ ID NO: 273 or fragments thereof.

The disclosure also provides an isolated polypeptide comprising an amino acid sequence of SEQ ID NO: 275 or fragments thereof.

The disclosure also provides an isolated polypeptide comprising an amino acid sequence of SEQ ID NO: 277 or fragments thereof.

The disclosure also provides an isolated polypeptide comprising an amino acid sequence of SEQ ID NO: 279 or fragments thereof.

The disclosure also provides an isolated polypeptide comprising an amino acid sequence of SEQ ID NO: 281 or fragments thereof.

The disclosure also provides an isolated polypeptide comprising an amino acid sequence of SEQ ID NO: 283 or fragments thereof.

The disclosure also provides an isolated polypeptide comprising an amino acid sequence of SEQ ID NO: 285 or fragments thereof.

The disclosure also provides an isolated polypeptide comprising an amino acid sequence of SEQ ID NO: 287 or fragments thereof.

The disclosure also provides an isolated polypeptide comprising an amino acid sequence of SEQ ID NO: 289 or fragments thereof.

The disclosure also provides an isolated polypeptide comprising an amino acid sequence of SEQ ID NO: 291 or fragments thereof.

The disclosure also provides an isolated polypeptide comprising an amino acid sequence of SEQ ID NO: 293 or fragments thereof.

The disclosure also provides an isolated polypeptide comprising an amino acid sequence of SEQ ID NO: 295 or fragments thereof.

The disclosure also provides an isolated polypeptide comprising an amino acid sequence of SEQ ID NO: 297 or fragments thereof.

The disclosure also provides an isolated polypeptide comprising an amino acid sequence of SEQ ID NO: 299 or fragments thereof.

The disclosure also provides an isolated polypeptide comprising an amino acid sequence of SEQ ID NO: 301 or fragments thereof.

The disclosure also provides an isolated polypeptide comprising an amino acid sequence of SEQ ID NO: 303 or fragments thereof.

The disclosure also provides an isolated polypeptide comprising an amino acid sequence of SEQ ID NO: 305 or fragments thereof.

The disclosure also provides an isolated polypeptide comprising an amino acid sequence of SEQ ID NO: 307 or fragments thereof.

The disclosure also provides an isolated polypeptide comprising an amino acid sequence of SEQ ID NO: 309 or fragments thereof.

The disclosure also provides an isolated polypeptide comprising an amino acid sequence of SEQ ID NO: 311 or fragments thereof.

The disclosure also provides an isolated polypeptide comprising an amino acid sequence of SEQ ID NO: 313 or fragments thereof.

The disclosure also provides an isolated polypeptide comprising an amino acid sequence of SEQ ID NO: 315 or fragments thereof.

The disclosure also provides an isolated polypeptide comprising an amino acid sequence of SEQ ID NO: 317 or fragments thereof.

The disclosure also provides an isolated polypeptide comprising an amino acid sequence of SEQ ID NO: 319 or fragments thereof.

The disclosure also provides an isolated polypeptide comprising an amino acid sequence of SEQ ID NO: 321 or fragments thereof.

The disclosure also provides an isolated polypeptide comprising an amino acid sequence of SEQ ID NO: 323 or fragments thereof.

The disclosure also provides an isolated polypeptide comprising an amino acid sequence of SEQ ID NO: 325 or fragments thereof.

The disclosure also provides an isolated polypeptide comprising an amino acid sequence of SEQ ID NO: 327 or fragments thereof.

The disclosure also provides an isolated polypeptide comprising an amino acid sequence of SEQ ID NO: 329 or fragments thereof.

The disclosure also provides an isolated polypeptide comprising an amino acid sequence of SEQ ID NO: 331 or fragments thereof.

The disclosure also provides an isolated polypeptide comprising an amino acid sequence of SEQ ID NO: 333 or fragments thereof.

The disclosure also provides an isolated polypeptide comprising an amino acid sequence of SEQ ID NO: 335 or fragments thereof.

The disclosure also provides an isolated polypeptide comprising an amino acid sequence of SEQ ID NO: 337 or fragments thereof.

The disclosure also provides an isolated polypeptide comprising an amino acid sequence of SEQ ID NO: 339 or fragments thereof.

The disclosure also provides an isolated polypeptide comprising an amino acid sequence of SEQ ID NO: 341 or fragments thereof.

The disclosure also provides an isolated polypeptide comprising an amino acid sequence of SEQ ID NO: 343 or fragments thereof.

The disclosure also provides an isolated polypeptide comprising an amino acid sequence of SEQ ID NO: 345 or fragments thereof.

The disclosure also provides an isolated polypeptide comprising an amino acid sequence of SEQ ID NO: 347 or fragments thereof.

The disclosure also provides an isolated polypeptide comprising an amino acid sequence of SEQ ID NO: 349 or fragments thereof.

The disclosure also provides an isolated polypeptide comprising an amino acid sequence of SEQ ID NO: 351 or fragments thereof.

The disclosure also provides an isolated polypeptide comprising an amino acid sequence of SEQ ID NO: 353 or fragments thereof.

The disclosure also provides an isolated polypeptide comprising an amino acid sequence of SEQ ID NO: 355 or fragments thereof.

The disclosure also provides an isolated polypeptide comprising an amino acid sequence of SEQ ID NO: 357 or fragments thereof.

The disclosure also provides an isolated polypeptide comprising an amino acid sequence of SEQ ID NO: 359 or fragments thereof.

The disclosure also provides an isolated polypeptide comprising an amino acid sequence of SEQ ID NO: 361 or fragments thereof.

The disclosure also provides an isolated polypeptide comprising an amino acid sequence of SEQ ID NO: 363 or fragments thereof.

The disclosure also provides an isolated polypeptide comprising an amino acid sequence of SEQ ID NO: 365 or fragments thereof.

The disclosure also provides an isolated polypeptide comprising an amino acid sequence of SEQ ID NO: 367 or fragments thereof.

The disclosure also provides an isolated polypeptide comprising an amino acid sequence of SEQ ID NO: 369 or fragments thereof.

The disclosure also provides an isolated polypeptide comprising an amino acid sequence of SEQ ID NO: 371 or fragments thereof.

The disclosure also provides an isolated polypeptide comprising an amino acid sequence of SEQ ID NO: 373 or fragments thereof.

The disclosure also provides an isolated polypeptide comprising an amino acid sequence of SEQ ID NO: 375 or fragments thereof.

The disclosure also provides an isolated polypeptide comprising an amino acid sequence of SEQ ID NO: 377 or fragments thereof.

The disclosure also provides an isolated polypeptide comprising an amino acid sequence of SEQ ID NO: 379 or fragments thereof.

The disclosure also provides an isolated polypeptide comprising an amino acid sequence of SEQ ID NO: 381 or fragments thereof.

The disclosure also provides an isolated polypeptide comprising an amino acid sequence of SEQ ID NO: 383 or fragments thereof.

The disclosure also provides an isolated polypeptide comprising an amino acid sequence of SEQ ID NO: 385 or fragments thereof.

The disclosure also provides an isolated polypeptide comprising an amino acid sequence of SEQ ID NO: 387 or fragments thereof.

The disclosure also provides an isolated polypeptide comprising an amino acid sequence of SEQ ID NO: 389 or fragments thereof.

The disclosure also provides an isolated polypeptide comprising an amino acid sequence of SEQ ID NO: 391 or fragments thereof.

The disclosure also provides an isolated polypeptide comprising an amino acid sequence of SEQ ID NO: 393 or fragments thereof.

The disclosure also provides an isolated polypeptide comprising an amino acid sequence of SEQ ID NO: 395 or fragments thereof.

The disclosure also provides an isolated polypeptide comprising an amino acid sequence of SEQ ID NO: 397 or fragments thereof.

The disclosure also provides an isolated polypeptide comprising an amino acid sequence of SEQ ID NO: 399 or fragments thereof.

The disclosure also provides an isolated polypeptide comprising an amino acid sequence of SEQ ID NO: 401 or fragments thereof.

The disclosure also provides an isolated polypeptide comprising an amino acid sequence of SEQ ID NO: 403 or fragments thereof.

The disclosure also provides an isolated polypeptide comprising an amino acid sequence of SEQ ID NO: 405 or fragments thereof.

The disclosure also provides an isolated polypeptide comprising an amino acid sequence of SEQ ID NO: 407 or fragments thereof.

The disclosure also provides an isolated polypeptide comprising an amino acid sequence of SEQ ID NO: 421 or fragments thereof.

In some embodiments, the fragments are about 6-25 amino acids in length.

In some embodiments, the fragments comprise at least 6 amino acids. In some embodiments, the fragments comprise at least 7 amino acids In some embodiments, the fragments comprise at least 8 amino acids. In some embodiments, the fragments comprise at least 9 amino acids. In some embodiments, the fragments comprise at least 10 amino acids. In some embodiments, the fragments comprise at least 11 amino acids. In some embodiments, the fragments comprise at least 12 amino acids. In some embodiments, the fragments comprise at least 13 amino acids. In some embodiments, the fragments comprise at least 14 amino acids. In some embodiments, the fragments comprise at least 15 amino acids. In some embodiments, the fragments comprise at least 16 amino acids. In some embodiments, the fragments comprise at least 17 amino acids. In some embodiments, the fragments comprise at least 18 amino acids. In some embodiments, the fragments comprise at least 19 amino acids. In some embodiments, the fragments comprise at least 20 amino acids. In some embodiments, the fragments comprise at least 21 amino acids. In some embodiments, the fragments comprise at least 22 amino acids. In some embodiments, the fragments comprise at least 23 amino acids. In some embodiments, the fragments comprise at least 24 amino acids. In some embodiments, the fragments comprise at least 25 amino acids. In some embodiments, the fragments comprise about 6 amino acids. In some embodiments, the fragments comprise about 7 amino acids. In some embodiments, the fragments comprise about 8 amino acids. In some embodiments, the fragments comprise about 9 amino acids. In some embodiments, the fragments comprise about 10 amino acids. In some embodiments, the fragments comprise about 11 amino acids. In some embodiments, the fragments comprise about 12 amino acids. In some embodiments, the fragments comprise about 13 amino acids. In some embodiments, the fragments comprise about 14 amino acids. In some embodiments, the fragments comprise about 15 amino acids. In some embodiments, the fragments comprise about 16 amino acids. In some embodiments, the fragments comprise about 17 amino acids. In some embodiments, the fragments comprise about 18 amino acids. In some embodiments, the fragments comprise about 19 amino acids. In some embodiments, the fragments comprise about 20 amino acids. In some embodiments, the fragments comprise about 21 amino acids. In some embodiments, the fragments comprise about 22 amino acids. In some embodiments, the fragments comprise about 23 amino acids. In some embodiments, the fragments comprise about 24 amino acids. In some embodiments, the fragments comprise about 25 amino acids. In some embodiments, the fragments comprise about 6-25 amino acids. In some embodiments, the fragments comprise about 7-25 amino acids. In some embodiments, the fragments comprise about 8-25 amino acids. In some embodiments, the fragments comprise about 8-24 amino acids. In some embodiments, the fragments comprise about 8-23 amino acids. In some embodiments, the fragments comprise about 8-22 amino acids. In some embodiments, the fragments comprise about 8-21 amino acids. In some embodiments, the fragments comprise about 8-20 amino acids. In some embodiments, the fragments comprise about 8-19 amino acids. In some embodiments, the fragments comprise about 8-18 amino acids. In some embodiments, the fragments comprise about 8-17 amino acids. In some embodiments, the fragments comprise about 8-16 amino acids. In some embodiments, the fragments comprise about 8-15 amino acids. In some embodiments, the fragments comprise about 8-14 amino acids. In some embodiments, the fragments comprise about 9-14 amino acids. In some embodiments, the fragments comprise about 9-13 amino acids. In some embodiments, the fragments comprise about 9-12 amino acids. In some embodiments, the fragments comprise about 9-11 amino acids. In some embodiments, the fragments comprise about 9-10 amino acids.

In some embodiments, the fragments are immunogenic fragments.

Immunogenic fragments in general are peptides that activate T cells, for example those that induce cytotoxic T cells when presented on MHC. Methods for assessing activation of T cells and/or induction of cytotoxic T lymphocytes are well known. In an exemplary assay, PBMCs isolated from a multiple myeloma patient are cultured in vitro in the presence of a test neoantigen or fragments thereof and IL-25. The cultures may be replenished periodically with IL-15 and IL-2 and cultured for an additional 12 days. On day 12, the cultures are re-stimulated with the test neoantigen or fragments thereof and the following day T cell activation may be assessed by measuring a percentage of IFNγ⁺TNAα⁺ CD8⁺ cells when compared to a control culture.

The polypeptides and the heterologous polypeptides of the disclosure comprise one or more multiple myeloma neoantigens described herein. The polypeptides and the heterologous polypeptides of the disclosure are useful in generating the recombinant viruses, the cells and the vaccines of the disclosure and proteinaceous molecules that specifically bind the one or more multiple myeloma neoantigens of the disclosure or may be used directly as therapeutic agents by delivering them to a subject having a multiple myeloma using various technologies. The two or more neoantigens (e.g. polypeptides) may be incorporated into the vaccine in any order using standard cloning methods.

Through the validation process, 115 neoantigen polypeptides of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, and 421 were identified as particularly useful to be included into a multiple myeloma vaccine based on their expression profile, prevalence and in vitro immunogenicity. It is expected that any combination of two or more of the 115 neoantigens can be utilized to generate a multiple myeloma vaccine that can be delivered to a subject utilizing any available delivery vehicles and any form available, such as peptides, DNA, RNA, replicons, or using viral delivery. The two or more neoantigens (e.g. polypeptides) may be incorporated into the vaccine in any order using standard cloning methods.

The two or more polypeptides may be assembled into heterologous polynucleotides encoding heterologous polypeptides in any order, and the polypeptide order may differ between the various delivery options. In general, assembly of the polypeptides into a particular order may be based on generating a minimum number of junctional epitopes utilizing known algorithms.

In some embodiments, the disclosre provides a polypeptide comprising one or more polypeptides selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, and 421, and fragments thereof.

The disclosure also provides a polypeptide comprising two or more tandem repeats of SEQ ID NOS; 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof. In some embodiments, the polypeptide comprises 2, 3, 4, 5, or more than 5 repeats of the polypeptides of the disclosure.

In some embodiments, the polypeptides are joined head to tail.

In some embodimemt, the polypeptides can be separated by a linker.

Exemplary linker sequences include AAY, RR, DPP, HHAA, HHA, HHL, RKSYL, RKSY, SSL, or REKR. In some embodiments, the linkers disclosed herein may comprise a protease cleavage site such that the heterologous polypeptides may be cleaved in vivo in a subject into peptide fragments comprising neoantigen sequences, resulting in improved immune response.

In some embodiment the polypeptides are joined to each other directly without a linker without a linker.

In some embodiments, the polypeptides of the disclosure may further comprise a leader sequence or T-cell enhancer sequence (TCE) at the N-terminus. Leader sequences can increase the expression and/or increase immunological response. Exemplary leader sequences include the α chain of the TCR receptor of T² lymphocytes (HAVT20) (MACPGFLWALVISTC LEFSMA; SEQ ID NO: 423), a ubiquitin signal sequence (Ubiq) (MQIFVKTLTGKTITLEVEP SDTIENVKAKIQDKEGIPPDQQRLIFAGKQLEDGRTLSDYNIQKESTLHLVLRLRGVR; SEQ ID NO: 424), or a T cell enhancer (TCE) sequence, such as a peptide fragment of length of 28aa from the mandarin fish invariant chain (MGQKEQIHTLQKNSERMSKQLTRSSQAV; SEQ ID NO: 425). It is believed that the leader sequences may help in increasing an immune response to the epitopes disclosed herein.

### Polynucleotides

The disclosure also provides polynucleotides that encode any of the polypeptides disclosed herein

In some embodiments, the disclosure provides an isolated polynucleotide encoding a polypeptide of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407,or 421, or fragments thereof.

The disclosure also provides an isolated polynucleotide encoding a polypeptide that is at least 90% identical to the polypeptide of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127,129,131,133,135,137,139,141,143,145,147,149,151,153,155,157,159,161,163,165, 167,169,171,173,175,177,179,181,183,185,187,189,191,193,195,197,199,201,203,205, 207,209,211,213,215,217,219,221,223,225,227,229,231,233,235,237,239,241,243,245, 247,249,251,253,255,257,259,261,263,265,267,269,271,273,275,277,279,281,283,285, 287,289,291,293,295,297,299,301,303,305,307,309,311,313,315,317,319,321,323,325, 327,329,331,333,335,337,339,341,343,345,347,349,351,353,355,357,359,361,363,365, 367,369,371,373,375,377,379,381,383,385,387,389,391,393,395,397,399,401,403,405, 407, or 421 or fragments thereof;

The disclosure also provides an isolated polynucleotide comprising a polynucleotide sequence of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, or 422, or fragments thereof.

The disclosure also provides an isolated polynucleotide comprising a polynucleotide sequence that is at least 90% identical to the polynucleotide sequence of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, or 422, or fragments thereof.

The disclosure also provides an isolated heterologous polynucleotide comprising two or more polynucleotides selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, and 422, and fragments thereof.

The disclosure also provides an isolated heterologous polynucleotide encoding a heterologous polypeptide comprising two or more polypeptides selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, and 421, and fragments thereof.

In some embodiments, the fragments comprise at least 18 nucleotides. In some embodiments, the fragments comprise at least 21 nucleotides. In some embodiments, the fragments comprise at least 24 nucleotides. In some embodiments, the fragments comprise at least 27 nucleotides. In some embodiments, the fragments comprise at least 30 nucleotides. In some embodiments, the fragments comprise at least 33 nucleotides. In some embodiments, the fragments comprise at least 36 nucleotides. In some embodiments, the fragments comprise at least 39 nucleotides. In some embodiments, the fragments comprise at least 42 nucleotides. In some embodiments, the fragments comprise at least 45 nucleotides. In some embodiments, the fragments comprise at least 48 nucleotides. In some embodiments, the fragments comprise at least 51 nucleotides. In some embodiments, the fragments comprise at least 54 nucleotides. In some embodiments, the fragments comprise at least 57 nucleotides. In some embodiments, the fragments comprise at least 60 nucleotides. In some embodiments, the fragments comprise at least 63 nucleotides. In some embodiments, the fragments comprise at least 66 nucleotides. In some embodiments, the fragments comprise at least 69 nucleotides. In some embodiments, the fragments comprise at least 72 nucleotides. In some embodiments, the fragments comprise at least 75 nucleotides. In some embodiments, the fragments comprise about 18 nucleotides. In some embodiments, the fragments comprise about 21 nucleotides. In some embodiments, the fragments comprise about 24 nucleotides. In some embodiments, the fragments comprise about 27 nucleotides. In some embodiments, the fragments comprise about 30 nucleotides. In some embodiments, the fragments comprise about 33 nucleotides. In some embodiments, the fragments comprise about 36 nucleotides. In some embodiments, the fragments comprise about 39 nucleotides. In some embodiments, the fragments comprise about 42 nucleotides. In some embodiments, the fragments comprise about 45 nucleotides. In some embodiments, the fragments comprise about 48 nucleotides. In some embodiments, the fragments comprise about 51 nucleotides. In some embodiments, the fragments comprise about 54 nucleotides. In some embodiments, the fragments comprise about 57 nucleotides. In some embodiments, the fragments comprise about 60 nucleotides. In some embodiments, the fragments comprise about 63 nucleotides. In some embodiments, the fragments comprise about 66 nucleotides. In some embodiments, the fragments comprise about 69 nucleotides. In some embodiments, the fragments comprise about 72 nucleotides. In some embodiments, the fragments comprise about 75 nucleotides. In some embodiments, the fragments comprise about 18-75 nucleotides. In some embodiments, the fragments comprise about 21-75 nucleotides. In some embodiments, the fragments comprise about 24-75 nucleotides. In some embodiments, the fragments comprise about 24-72 nucleotides. In some embodiments, the fragments comprise about 24-69 nucleotides. In some embodiments, the fragments comprise about 24-66 nucleotides. In some embodiments, the fragments comprise about 24-63 nucleotides. In some embodiments, the fragments comprise about 24-60 nucleotides. In some embodiments, the fragments comprise about 24-57 nucleotides. In some embodiments, the fragments comprise about 24-54 nucleotides. In some embodiments, the fragments comprise about 24-51 nucleotides. In some embodiments, the fragments comprise about 24-48 nucleotides. In some embodiments, the fragments comprise about 24-45 nucleotides. In some embodiments, the fragments comprise about 24-42 nucleotides. In some embodiments, the fragments comprise about 27-42 nucleotides. In some embodiments, the fragments comprise about 27-39 nucleotides. In some embodiments, the fragments comprise about 27-36 nucleotides. In some embodiments, the fragments comprise about 27-33 nucleotides. In some embodiments, the fragments comprise about 27-30 nucleotides.

The polynucleotides and the heterologous polynucleotides of the disclosure encode the multiple myeloma neoantigens and heterologous polypeptides comprising two or more multiple myeloma neoantigens described herein. The polynucleotides and the heterologous polynucleotides of the disclosure are useful in generating the polypeptides, the heterologous polypeptides, the vectors, the recombinant viruses, the cells and the vaccines of the disclosure. The polynucleotides and the heterologous polynucleotides of the disclosure may be utilized as therapeutics by delivering them to a subject having a multiple myeloma using various technologies, including viral vectors as described herein or other delivery technologies as also described herein.

The disclosure also provides an isolated polynucleotide encoding the polypeptide of SEQ ID NO: 1 or fragments thereof. In some embodiments, the polypeptide of SEQ ID NO: 1, or a fragment thereof, is encoded by the polynucleotide of SEQ ID NO: 2 or fragments thereof.

The disclosure also provides an isolated polynucleotide encoding the polypeptide of SEQ ID NO: 3 or fragments thereof. In some embodiments, the polypeptide of SEQ ID NO: 3, or a fragment thereof, is encoded by the polynucleotide of SEQ ID NO: 4 or fragments thereof.

The disclosure also provides an isolated polynucleotide encoding the polypeptide of SEQ ID NO: 5 or fragments thereof. In some embodiments, the polypeptide of SEQ ID NO: 5, or a fragment thereof, is encoded by the polynucleotide of SEQ ID NO: 6 or fragments thereof.

The disclosure also provides an isolated polynucleotide encoding the polypeptide of SEQ ID NO: 7 or fragments thereof. In some embodiments, the polypeptide of SEQ ID NO: 7, or a fragment thereof, is encoded by the polynucleotide of SEQ ID NO: 8 or fragments thereof.

The disclosure also provides an isolated polynucleotide encoding the polypeptide of SEQ ID NO: 9 or fragments thereof. In some embodiments, the polypeptide of SEQ ID NO: 9, or a fragment thereof, is encoded by the polynucleotide of SEQ ID NO: 10 or fragments thereof.

The disclosure also provides an isolated polynucleotide encoding the polypeptide of SEQ ID NO: 11 or fragments thereof. In some embodiments, the polypeptide of SEQ ID NO: 11, or a fragment thereof, is encoded by the polynucleotide of SEQ ID NO: 12 or fragments thereof.

The disclosure also provides an isolated polynucleotide encoding the polypeptide of SEQ ID NO: 13 or fragments thereof. In some embodiments, the polypeptide of SEQ ID NO: 13, or a fragment thereof, is encoded by the polynucleotide of SEQ ID NO: 14 or fragments thereof.

The disclosure also provides an isolated polynucleotide encoding the polypeptide of SEQ ID NO: 15 or fragments thereof. In some embodiments, the polypeptide of SEQ ID NO: 15, or a fragment thereof, is encoded by the polynucleotide of SEQ ID NO: 16 or fragments thereof.

The disclosure also provides an isolated polynucleotide encoding the polypeptide of SEQ ID NO: 17 or fragments thereof. In some embodiments, the polypeptide of SEQ ID NO: 17, or a fragment thereof, is encoded by the polynucleotide of SEQ ID NO: 18 or fragments thereof.

The disclosure also provides an isolated polynucleotide encoding the polypeptide of SEQ ID NO: 19 or fragments thereof. In some embodiments, the polypeptide of SEQ ID NO: 19, or a fragment thereof, is encoded by the polynucleotide of SEQ ID NO: 20 or fragments thereof.

The disclosure also provides an isolated polynucleotide encoding the polypeptide of SEQ ID NO: 21 or fragments thereof. In some embodiments, the polypeptide of SEQ ID NO: 21, or a fragment thereof, is encoded by the polynucleotide of SEQ ID NO: 22 or fragments thereof.

The disclosure also provides an isolated polynucleotide encoding the polypeptide of SEQ ID NO: 23 or fragments thereof. In some embodiments, the polypeptide of SEQ ID NO: 23, or a fragment thereof, is encoded by the polynucleotide of SEQ ID NO: 24 or fragments thereof.

The disclosure also provides an isolated polynucleotide encoding the polypeptide of SEQ ID NO: 25 or fragments thereof. In some embodiments, the polypeptide of SEQ ID NO: 25, or a fragment thereof, is encoded by the polynucleotide of SEQ ID NO: 26 or fragments thereof.

The disclosure also provides an isolated polynucleotide encoding the polypeptide of SEQ ID NO: 27 or fragments thereof. In some embodiments, the polypeptide of SEQ ID NO: 27, or a fragment thereof, is encoded by the polynucleotide of SEQ ID NO: 28 or fragments thereof.

The disclosure also provides an isolated polynucleotide encoding the polypeptide of SEQ ID NO: 29 or fragments thereof. In some embodiments, the polypeptide of SEQ ID NO: 29, or a fragment thereof, is encoded by the polynucleotide of SEQ ID NO: 30 or fragments thereof.

The disclosure also provides an isolated polynucleotide encoding the polypeptide of SEQ ID NO: 31 or fragments thereof. In some embodiments, the polypeptide of SEQ ID NO: 31, or a fragment thereof, is encoded by the polynucleotide of SEQ ID NO: 32 or fragments thereof.

The disclosure also provides an isolated polynucleotide encoding the polypeptide of SEQ ID NO: 33 or fragments thereof. In some embodiments, the polypeptide of SEQ ID NO: 33, or a fragment thereof, is encoded by the polynucleotide of SEQ ID NO: 34 or fragments thereof.

The disclosure also provides an isolated polynucleotide encoding the polypeptide of SEQ ID NO: 35 or fragments thereof. In some embodiments, the polypeptide of SEQ ID NO: 35, or a fragment thereof, is encoded by the polynucleotide of SEQ ID NO: 36 or fragments thereof.

The disclosure also provides an isolated polynucleotide encoding the polypeptide of SEQ ID NO: 37 or fragments thereof. In some embodiments, the polypeptide of SEQ ID NO: 37, or a fragment thereof, is encoded by the polynucleotide of SEQ ID NO: 38 or fragments thereof.

The disclosure also provides an isolated polynucleotide encoding the polypeptide of SEQ ID NO: 39 or fragments thereof. In some embodiments, the polypeptide of SEQ ID NO: 39, or a fragment thereof, is encoded by the polynucleotide of SEQ ID NO: 40 or fragments thereof.

The disclosure also provides an isolated polynucleotide encoding the polypeptide of SEQ ID NO: 41 or fragments thereof. In some embodiments, the polypeptide of SEQ ID NO: 41, or a fragment thereof, is encoded by the polynucleotide of SEQ ID NO: 42 or fragments thereof.

The disclosure also provides an isolated polynucleotide encoding the polypeptide of SEQ ID NO: 43 or fragments thereof. In some embodiments, the polypeptide of SEQ ID NO: 43, or a fragment thereof, is encoded by the polynucleotide of SEQ ID NO: 44 or fragments thereof.

The disclosure also provides an isolated polynucleotide encoding the polypeptide of SEQ ID NO: 45 or fragments thereof. In some embodiments, the polypeptide of SEQ ID NO: 45, or a fragment thereof, is encoded by the polynucleotide of SEQ ID NO: 46 or fragments thereof.

The disclosure also provides an isolated polynucleotide encoding the polypeptide of SEQ ID NO: 47 or fragments thereof. In some embodiments, the polypeptide of SEQ ID NO: 47, or a fragment thereof, is encoded by the polynucleotide of SEQ ID NO: 48 or fragments thereof.

The disclosure also provides an isolated polynucleotide encoding the polypeptide of SEQ ID NO: 49 or fragments thereof. In some embodiments, the polypeptide of SEQ ID NO: 49, or a fragment thereof, is encoded by the polynucleotide of SEQ ID NO: 50 or fragments thereof.

The disclosure also provides an isolated polynucleotide encoding the polypeptide of SEQ ID NO: 51 or fragments thereof. In some embodiments, the polypeptide of SEQ ID NO: 51, or a fragment thereof, is encoded by the polynucleotide of SEQ ID NO: 52 or fragments thereof.

The disclosure also provides an isolated polynucleotide encoding the polypeptide of SEQ ID NO: 53 or fragments thereof. In some embodiments, the polypeptide of SEQ ID NO: 53, or a fragment thereof, is encoded by the polynucleotide of SEQ ID NO: 54 or fragments thereof.

The disclosure also provides an isolated polynucleotide encoding the polypeptide of SEQ ID NO: 55 or fragments thereof. In some embodiments, the polypeptide of SEQ ID NO: 55, or a fragment thereof, is encoded by the polynucleotide of SEQ ID NO: 56 or fragments thereof.

The disclosure also provides an isolated polynucleotide encoding the polypeptide of SEQ ID NO: 57 or fragments thereof. In some embodiments, the polypeptide of SEQ ID NO: 57, or a fragment thereof, is encoded by the polynucleotide of SEQ ID NO: 58 or fragments thereof.

The disclosure also provides an isolated polynucleotide encoding the polypeptide of SEQ ID NO: 59 or fragments thereof. In some embodiments, the polypeptide of SEQ ID NO: 59, or a fragment thereof, is encoded by the polynucleotide of SEQ ID NO: 60 or fragments thereof.

The disclosure also provides an isolated polynucleotide encoding the polypeptide of SEQ ID NO: 61 or fragments thereof. In some embodiments, the polypeptide of SEQ ID NO: 61, or a fragment thereof, is encoded by the polynucleotide of SEQ ID NO: 62 or fragments thereof.

The disclosure also provides an isolated polynucleotide encoding the polypeptide of SEQ ID NO: 63 or fragments thereof. In some embodiments, the polypeptide of SEQ ID NO: 63, or a fragment thereof, is encoded by the polynucleotide of SEQ ID NO: 64 or fragments thereof.

The disclosure also provides an isolated polynucleotide encoding the polypeptide of SEQ ID NO: 65 or fragments thereof. In some embodiments, the polypeptide of SEQ ID NO: 65, or a fragment thereof, is encoded by the polynucleotide of SEQ ID NO: 66 or fragments thereof.

The disclosure also provides an isolated polynucleotide encoding the polypeptide of SEQ ID NO: 67 or fragments thereof. In some embodiments, the polypeptide of SEQ ID NO: 67, or a fragment thereof, is encoded by the polynucleotide of SEQ ID NO: 68 or fragments thereof.

The disclosure also provides an isolated polynucleotide encoding the polypeptide of SEQ ID NO: 69 or fragments thereof. In some embodiments, the polypeptide of SEQ ID NO: 69, or a fragment thereof, is encoded by the polynucleotide of SEQ ID NO: 70 or fragments thereof.

The disclosure also provides an isolated polynucleotide encoding the polypeptide of SEQ ID NO: 71 or fragments thereof. In some embodiments, the polypeptide of SEQ ID NO: 71, or a fragment thereof, is encoded by the polynucleotide of SEQ ID NO: 72 or fragments thereof.

The disclosure also provides an isolated polynucleotide encoding the polypeptide of SEQ ID NO: 73 or fragments thereof. In some embodiments, the polypeptide of SEQ ID NO: 73, or a fragment thereof, is encoded by the polynucleotide of SEQ ID NO: 74 or fragments thereof.

The disclosure also provides an isolated polynucleotide encoding the polypeptide of SEQ ID NO: 75 or fragments thereof. In some embodiments, the polypeptide of SEQ ID NO: 75, or a fragment thereof, is encoded by the polynucleotide of SEQ ID NO: 76 or fragments thereof.

The disclosure also provides an isolated polynucleotide encoding the polypeptide of SEQ ID NO: 77 or fragments thereof. In some embodiments, the polypeptide of SEQ ID NO: 77, or a fragment thereof, is encoded by the polynucleotide of SEQ ID NO: 78 or fragments thereof.

The disclosure also provides an isolated polynucleotide encoding the polypeptide of SEQ ID NO: 79 or fragments thereof. In some embodiments, the polypeptide of SEQ ID NO: 79, or a fragment thereof, is encoded by the polynucleotide of SEQ ID NO: 80 or fragments thereof.

The disclosure also provides an isolated polynucleotide encoding the polypeptide of SEQ ID NO: 81 or fragments thereof. In some embodiments, the polypeptide of SEQ ID NO: 81, or a fragment thereof, is encoded by the polynucleotide of SEQ ID NO: 82 or fragments thereof.

The disclosure also provides an isolated polynucleotide encoding the polypeptide of SEQ ID NO: 83 or fragments thereof. In some embodiments, the polypeptide of SEQ ID NO: 83, or a fragment thereof, is encoded by the polynucleotide of SEQ ID NO: 84 or fragments thereof.

The disclosure also provides an isolated polynucleotide encoding the polypeptide of SEQ ID NO: 85 or fragments thereof. In some embodiments, the polypeptide of SEQ ID NO: 85, or a fragment thereof, is encoded by the polynucleotide of SEQ ID NO: 86 or fragments thereof.

The disclosure also provides an isolated polynucleotide encoding the polypeptide of SEQ ID NO: 87 or fragments thereof. In some embodiments, the polypeptide of SEQ ID NO: 87, or a fragment thereof, is encoded by the polynucleotide of SEQ ID NO: 88 or fragments thereof.

The disclosure also provides an isolated polynucleotide encoding the polypeptide of SEQ ID NO: 89 or fragments thereof. In some embodiments, the polypeptide of SEQ ID NO: 89, or a fragment thereof, is encoded by the polynucleotide of SEQ ID NO: 90 or fragments thereof.

The disclosure also provides an isolated polynucleotide encoding the polypeptide of SEQ ID NO: 91 or fragments thereof. In some embodiments, the polypeptide of SEQ ID NO: 91, or a fragment thereof, is encoded by the polynucleotide of SEQ ID NO: 92 or fragments thereof.

The disclosure also provides an isolated polynucleotide encoding the polypeptide of SEQ ID NO: 93 or fragments thereof. In some embodiments, the polypeptide of SEQ ID NO: 93, or a fragment thereof, is encoded by the polynucleotide of SEQ ID NO: 94 or fragments thereof.

The disclosure also provides an isolated polynucleotide encoding the polypeptide of SEQ ID NO: 95 or fragments thereof. In some embodiments, the polypeptide of SEQ ID NO: 95, or a fragment thereof, is encoded by the polynucleotide of SEQ ID NO: 96 or fragments thereof.

The disclosure also provides an isolated polynucleotide encoding the polypeptide of SEQ ID NO: 97 or fragments thereof. In some embodiments, the polypeptide of SEQ ID NO: 97, or a fragment thereof, is encoded by the polynucleotide of SEQ ID NO: 98 or fragments thereof.

The disclosure also provides an isolated polynucleotide encoding the polypeptide of SEQ ID NO: 99 or fragments thereof. In some embodiments, the polypeptide of SEQ ID NO: 99, or a fragment thereof, is encoded by the polynucleotide of SEQ ID NO: 100 or fragments thereof.

The disclosure also provides an isolated polynucleotide encoding the polypeptide of SEQ ID NO: 101 or fragments thereof. In some embodiments, the polypeptide of SEQ ID NO: 101, or a fragment thereof, is encoded by the polynucleotide of SEQ ID NO: 102 or fragments thereof.

The disclosure also provides an isolated polynucleotide encoding the polypeptide of SEQ ID NO: 103 or fragments thereof. In some embodiments, the polypeptide of SEQ ID NO: 103, or a fragment thereof, is encoded by the polynucleotide of SEQ ID NO: 104 or fragments thereof.

The disclosure also provides an isolated polynucleotide encoding the polypeptide of SEQ ID NO: 105 or fragments thereof. In some embodiments, the polypeptide of SEQ ID NO: 105, or a fragment thereof, is encoded by the polynucleotide of SEQ ID NO: 106 or fragments thereof.

The disclosure also provides an isolated polynucleotide encoding the polypeptide of SEQ ID NO: 107 or fragments thereof. In some embodiments, the polypeptide of SEQ ID NO: 107, or a fragment thereof, is encoded by the polynucleotide of SEQ ID NO: 108 or fragments thereof.

The disclosure also provides an isolated polynucleotide encoding the polypeptide of SEQ ID NO: 109 or fragments thereof. In some embodiments, the polypeptide of SEQ ID NO: 109, or a fragment thereof, is encoded by the polynucleotide of SEQ ID NO: 110 or fragments thereof.

The disclosure also provides an isolated polynucleotide encoding the polypeptide of SEQ ID NO: 111 or fragments thereof. In some embodiments, the polypeptide of SEQ ID NO: 111, or a fragment thereof, is encoded by the polynucleotide of SEQ ID NO: 112 or fragments thereof.

The disclosure also provides an isolated polynucleotide encoding the polypeptide of SEQ ID NO: 113 or fragments thereof. In some embodiments, the polypeptide of SEQ ID NO: 113, or a fragment thereof, is encoded by the polynucleotide of SEQ ID NO: 114 or fragments thereof.

The disclosure also provides an isolated polynucleotide encoding the polypeptide of SEQ ID NO: 115 or fragments thereof. In some embodiments, the polypeptide of SEQ ID NO: 115, or a fragment thereof, is encoded by the polynucleotide of SEQ ID NO: 116 or fragments thereof.

The disclosure also provides an isolated polynucleotide encoding the polypeptide of SEQ ID NO: 117 or fragments thereof. In some embodiments, the polypeptide of SEQ ID NO: 117, or a fragment thereof, is encoded by the polynucleotide of SEQ ID NO: 118 or fragments thereof.

The disclosure also provides an isolated polynucleotide encoding the polypeptide of SEQ ID NO: 119 or fragments thereof. In some embodiments, the polypeptide of SEQ ID NO: 119, or a fragment thereof, is encoded by the polynucleotide of SEQ ID NO: 120 or fragments thereof.

The disclosure also provides an isolated polynucleotide encoding the polypeptide of SEQ ID NO: 121 or fragments thereof. In some embodiments, the polypeptide of SEQ ID NO: 121, or a fragment thereof, is encoded by the polynucleotide of SEQ ID NO: 122 or fragments thereof.

The disclosure also provides an isolated polynucleotide encoding the polypeptide of SEQ ID NO: 123 or fragments thereof. In some embodiments, the polypeptide of SEQ ID NO: 123, or a fragment thereof, is encoded by the polynucleotide of SEQ ID NO: 124 or fragments thereof.

The disclosure also provides an isolated polynucleotide encoding the polypeptide of SEQ ID NO: 125 or fragments thereof. In some embodiments, the polypeptide of SEQ ID NO: 125, or a fragment thereof, is encoded by the polynucleotide of SEQ ID NO: 126 or fragments thereof.

The disclosure also provides an isolated polynucleotide encoding the polypeptide of SEQ ID NO: 127 or fragments thereof. In some embodiments, the polypeptide of SEQ ID NO: 127, or a fragment thereof, is encoded by the polynucleotide of SEQ ID NO: 128 or fragments thereof.

The disclosure also provides an isolated polynucleotide encoding the polypeptide of SEQ ID NO: 129 or fragments thereof. In some embodiments, the polypeptide of SEQ ID NO: 129, or a fragment thereof, is encoded by the polynucleotide of SEQ ID NO: 130 or fragments thereof.

The disclosure also provides an isolated polynucleotide encoding the polypeptide of SEQ ID NO: 131 or fragments thereof. In some embodiments, the polypeptide of SEQ ID NO: 131, or a fragment thereof, is encoded by the polynucleotide of SEQ ID NO: 132 or fragments thereof.

The disclosure also provides an isolated polynucleotide encoding the polypeptide of SEQ ID NO: 133 or fragments thereof. In some embodiments, the polypeptide of SEQ ID NO: 133, or a fragment thereof, is encoded by the polynucleotide of SEQ ID NO: 134 or fragments thereof.

The disclosure also provides an isolated polynucleotide encoding the polypeptide of SEQ ID NO: 135 or fragments thereof. In some embodiments, the polypeptide of SEQ ID NO: 135, or a fragment thereof, is encoded by the polynucleotide of SEQ ID NO: 136 or fragments thereof.

The disclosure also provides an isolated polynucleotide encoding the polypeptide of SEQ ID NO: 137 or fragments thereof. In some embodiments, the polypeptide of SEQ ID NO: 137, or a fragment thereof, is encoded by the polynucleotide of SEQ ID NO: 138 or fragments thereof.

The disclosure also provides an isolated polynucleotide encoding the polypeptide of SEQ ID NO: 139 or fragments thereof. In some embodiments, the polypeptide of SEQ ID NO: 139, or a fragment thereof, is encoded by the polynucleotide of SEQ ID NO: 140 or fragments thereof.

The disclosure also provides an isolated polynucleotide encoding the polypeptide of SEQ ID NO: 141 or fragments thereof. In some embodiments, the polypeptide of SEQ ID NO: 141, or a fragment thereof, is encoded by the polynucleotide of SEQ ID NO: 142 or fragments thereof.

The disclosure also provides an isolated polynucleotide encoding the polypeptide of SEQ ID NO: 143 or fragments thereof. In some embodiments, the polypeptide of SEQ ID NO: 143, or a fragment thereof, is encoded by the polynucleotide of SEQ ID NO: 144 or fragments thereof.

The disclosure also provides an isolated polynucleotide encoding the polypeptide of SEQ ID NO: 145 or fragments thereof. In some embodiments, the polypeptide of SEQ ID NO: 145, or a fragment thereof, is encoded by the polynucleotide of SEQ ID NO: 146 or fragments thereof.

The disclosure also provides an isolated polynucleotide encoding the polypeptide of SEQ ID NO: 147 or fragments thereof. In some embodiments, the polypeptide of SEQ ID NO: 147, or a fragment thereof, is encoded by the polynucleotide of SEQ ID NO: 148 or fragments thereof.

The disclosure also provides an isolated polynucleotide encoding the polypeptide of SEQ ID NO: 149 or fragments thereof. In some embodiments, the polypeptide of SEQ ID NO: 149, or a fragment thereof, is encoded by the polynucleotide of SEQ ID NO: 150 or fragments thereof.

The disclosure also provides an isolated polynucleotide encoding the polypeptide of SEQ ID NO: 151 or fragments thereof. In some embodiments, the polypeptide of SEQ ID NO: 151, or a fragment thereof, is encoded by the polynucleotide of SEQ ID NO: 152 or fragments thereof.

The disclosure also provides an isolated polynucleotide encoding the polypeptide of SEQ ID NO: 153 or fragments thereof. In some embodiments, the polypeptide of SEQ ID NO: 153, or a fragment thereof, is encoded by the polynucleotide of SEQ ID NO: 154 or fragments thereof.

The disclosure also provides an isolated polynucleotide encoding the polypeptide of SEQ ID NO: 155 or fragments thereof. In some embodiments, the polypeptide of SEQ ID NO: 155, or a fragment thereof, is encoded by the polynucleotide of SEQ ID NO: 156 or fragments thereof.

The disclosure also provides an isolated polynucleotide encoding the polypeptide of SEQ ID NO: 157 or fragments thereof. In some embodiments, the polypeptide of SEQ ID NO: 157, or a fragment thereof, is encoded by the polynucleotide of SEQ ID NO: 158 or fragments thereof.

The disclosure also provides an isolated polynucleotide encoding the polypeptide of SEQ ID NO: 159 or fragments thereof. In some embodiments, the polypeptide of SEQ ID NO: 159, or a fragment thereof, is encoded by the polynucleotide of SEQ ID NO: 160 or fragments thereof.

The disclosure also provides an isolated polynucleotide encoding the polypeptide of SEQ ID NO: 161 or fragments thereof. In some embodiments, the polypeptide of SEQ ID NO: 161, or a fragment thereof, is encoded by the polynucleotide of SEQ ID NO: 162 or fragments thereof.

The disclosure also provides an isolated polynucleotide encoding the polypeptide of SEQ ID NO: 163 or fragments thereof. In some embodiments, the polypeptide of SEQ ID NO: 163, or a fragment thereof, is encoded by the polynucleotide of SEQ ID NO: 164 or fragments thereof.

The disclosure also provides an isolated polynucleotide encoding the polypeptide of SEQ ID NO: 165 or fragments thereof. In some embodiments, the polypeptide of SEQ ID NO: 165, or a fragment thereof, is encoded by the polynucleotide of SEQ ID NO: 166 or fragments thereof.

The disclosure also provides an isolated polynucleotide encoding the polypeptide of SEQ ID NO: 167 or fragments thereof. In some embodiments, the polypeptide of SEQ ID NO: 167, or a fragment thereof, is encoded by the polynucleotide of SEQ ID NO: 168 or fragments thereof.

The disclosure also provides an isolated polynucleotide encoding the polypeptide of SEQ ID NO: 169 or fragments thereof. In some embodiments, the polypeptide of SEQ ID NO: 169, or a fragment thereof, is encoded by the polynucleotide of SEQ ID NO: 170 or fragments thereof.

The disclosure also provides an isolated polynucleotide encoding the polypeptide of SEQ ID NO: 171 or fragments thereof. In some embodiments, the polypeptide of SEQ ID NO: 171, or a fragment thereof, is encoded by the polynucleotide of SEQ ID NO: 172 or fragments thereof.

The disclosure also provides an isolated polynucleotide encoding the polypeptide of SEQ ID NO: 173 or fragments thereof. In some embodiments, the polypeptide of SEQ ID NO: 173, or a fragment thereof, is encoded by the polynucleotide of SEQ ID NO: 174 or fragments thereof.

The disclosure also provides an isolated polynucleotide encoding the polypeptide of SEQ ID NO: 175 or fragments thereof. In some embodiments, the polypeptide of SEQ ID NO: 175, or a fragment thereof, is encoded by the polynucleotide of SEQ ID NO: 176 or fragments thereof.

The disclosure also provides an isolated polynucleotide encoding the polypeptide of SEQ ID NO: 177 or fragments thereof. In some embodiments, the polypeptide of SEQ ID NO: 177, or a fragment thereof, is encoded by the polynucleotide of SEQ ID NO: 178 or fragments thereof.

The disclosure also provides an isolated polynucleotide encoding the polypeptide of SEQ ID NO: 179 or fragments thereof. In some embodiments, the polypeptide of SEQ ID NO: 179, or a fragment thereof, is encoded by the polynucleotide of SEQ ID NO: 180 or fragments thereof.

The disclosure also provides an isolated polynucleotide encoding the polypeptide of SEQ ID NO: 181 or fragments thereof. In some embodiments, the polypeptide of SEQ ID NO: 181, or a fragment thereof, is encoded by the polynucleotide of SEQ ID NO: 182 or fragments thereof.

The disclosure also provides an isolated polynucleotide encoding the polypeptide of SEQ ID NO: 183 or fragments thereof. In some embodiments, the polypeptide of SEQ ID NO: 183, or a fragment thereof, is encoded by the polynucleotide of SEQ ID NO: 184 or fragments thereof.

The disclosure also provides an isolated polynucleotide encoding the polypeptide of SEQ ID NO: 185 or fragments thereof. In some embodiments, the polypeptide of SEQ ID NO: 185, or a fragment thereof, is encoded by the polynucleotide of SEQ ID NO: 186 or fragments thereof.

The disclosure also provides an isolated polynucleotide encoding the polypeptide of SEQ ID NO: 187 or fragments thereof. In some embodiments, the polypeptide of SEQ ID NO: 187, or a fragment thereof, is encoded by the polynucleotide of SEQ ID NO: 188 or fragments thereof.

The disclosure also provides an isolated polynucleotide encoding the polypeptide of SEQ ID NO: 189 or fragments thereof. In some embodiments, the polypeptide of SEQ ID NO: 189, or a fragment thereof, is encoded by the polynucleotide of SEQ ID NO: 190 or fragments thereof.

The disclosure also provides an isolated polynucleotide encoding the polypeptide of SEQ ID NO: 191 or fragments thereof. In some embodiments, the polypeptide of SEQ ID NO: 191, or a fragment thereof, is encoded by the polynucleotide of SEQ ID NO: 192 or fragments thereof.

The disclosure also provides an isolated polynucleotide encoding the polypeptide of SEQ ID NO: 193 or fragments thereof. In some embodiments, the polypeptide of SEQ ID NO: 193, or a fragment thereof, is encoded by the polynucleotide of SEQ ID NO: 194 or fragments thereof.

The disclosure also provides an isolated polynucleotide encoding the polypeptide of SEQ ID NO: 195 or fragments thereof. In some embodiments, the polypeptide of SEQ ID NO: 195, or a fragment thereof, is encoded by the polynucleotide of SEQ ID NO: 196 or fragments thereof.

The disclosure also provides an isolated polynucleotide encoding the polypeptide of SEQ ID NO: 197 or fragments thereof. In some embodiments, the polypeptide of SEQ ID NO: 197, or a fragment thereof, is encoded by the polynucleotide of SEQ ID NO: 198 or fragments thereof.

The disclosure also provides an isolated polynucleotide encoding the polypeptide of SEQ ID NO: 199 or fragments thereof. In some embodiments, the polypeptide of SEQ ID NO: 199, or a fragment thereof, is encoded by the polynucleotide of SEQ ID NO: 200 or fragments thereof.

The disclosure also provides an isolated polynucleotide encoding the polypeptide of SEQ ID NO: 201 or fragments thereof. In some embodiments, the polypeptide of SEQ ID NO: 201, or a fragment thereof, is encoded by the polynucleotide of SEQ ID NO: 202 or fragments thereof.

The disclosure also provides an isolated polynucleotide encoding the polypeptide of SEQ ID NO: 203 or fragments thereof. In some embodiments, the polypeptide of SEQ ID NO: 203, or a fragment thereof, is encoded by the polynucleotide of SEQ ID NO: 204 or fragments thereof.

The disclosure also provides an isolated polynucleotide encoding the polypeptide of SEQ ID NO: 205 or fragments thereof. In some embodiments, the polypeptide of SEQ ID NO: 205, or a fragment thereof, is encoded by the polynucleotide of SEQ ID NO: 206 or fragments thereof.

The disclosure also provides an isolated polynucleotide encoding the polypeptide of SEQ ID NO: 207 or fragments thereof. In some embodiments, the polypeptide of SEQ ID NO: 207, or a fragment thereof, is encoded by the polynucleotide of SEQ ID NO: 208 or fragments thereof.

The disclosure also provides an isolated polynucleotide encoding the polypeptide of SEQ ID NO: 209 or fragments thereof. In some embodiments, the polypeptide of SEQ ID NO: 209, or a fragment thereof, is encoded by the polynucleotide of SEQ ID NO: 210 or fragments thereof.

The disclosure also provides an isolated polynucleotide encoding the polypeptide of SEQ ID NO: 211 or fragments thereof. In some embodiments, the polypeptide of SEQ ID NO: 211, or a fragment thereof, is encoded by the polynucleotide of SEQ ID NO: 212 or fragments thereof.

The disclosure also provides an isolated polynucleotide encoding the polypeptide of SEQ ID NO: 213 or fragments thereof. In some embodiments, the polypeptide of SEQ ID NO: 213, or a fragment thereof, is encoded by the polynucleotide of SEQ ID NO: 214 or fragments thereof.

The disclosure also provides an isolated polynucleotide encoding the polypeptide of SEQ ID NO: 215 or fragments thereof. In some embodiments, the polypeptide of SEQ ID NO: 215, or a fragment thereof, is encoded by the polynucleotide of SEQ ID NO: 216 or fragments thereof.

The disclosure also provides an isolated polynucleotide encoding the polypeptide of SEQ ID NO: 217 or fragments thereof. In some embodiments, the polypeptide of SEQ ID NO: 217, or a fragment thereof, is encoded by the polynucleotide of SEQ ID NO: 218 or fragments thereof.

The disclosure also provides an isolated polynucleotide encoding the polypeptide of SEQ ID NO: 219 or fragments thereof. In some embodiments, the polypeptide of SEQ ID NO: 219, or a fragment thereof, is encoded by the polynucleotide of SEQ ID NO: 220 or fragments thereof.

The disclosure also provides an isolated polynucleotide encoding the polypeptide of SEQ ID NO: 221 or fragments thereof. In some embodiments, the polypeptide of SEQ ID NO: 221, or a fragment thereof, is encoded by the polynucleotide of SEQ ID NO: 222 or fragments thereof.

The disclosure also provides an isolated polynucleotide encoding the polypeptide of SEQ ID NO: 223 or fragments thereof. In some embodiments, the polypeptide of SEQ ID NO: 223, or a fragment thereof, is encoded by the polynucleotide of SEQ ID NO: 224 or fragments thereof.

The disclosure also provides an isolated polynucleotide encoding the polypeptide of SEQ ID NO: 225 or fragments thereof. In some embodiments, the polypeptide of SEQ ID NO: 225, or a fragment thereof, is encoded by the polynucleotide of SEQ ID NO: 226 or fragments thereof.

The disclosure also provides an isolated polynucleotide encoding the polypeptide of SEQ ID NO: 227 or fragments thereof. In some embodiments, the polypeptide of SEQ ID NO: 227, or a fragment thereof, is encoded by the polynucleotide of SEQ ID NO: 228 or fragments thereof.

The disclosure also provides an isolated polynucleotide encoding the polypeptide of SEQ ID NO: 229 or fragments thereof. In some embodiments, the polypeptide of SEQ ID NO: 229, or a fragment thereof, is encoded by the polynucleotide of SEQ ID NO: 230 or fragments thereof.

The disclosure also provides an isolated polynucleotide encoding the polypeptide of SEQ ID NO: 231 or fragments thereof. In some embodiments, the polypeptide of SEQ ID NO: 231, or a fragment thereof, is encoded by the polynucleotide of SEQ ID NO: 232 or fragments thereof.

The disclosure also provides an isolated polynucleotide encoding the polypeptide of SEQ ID NO: 233 or fragments thereof. In some embodiments, the polypeptide of SEQ ID NO: 233, or a fragment thereof, is encoded by the polynucleotide of SEQ ID NO: 234 or fragments thereof.

The disclosure also provides an isolated polynucleotide encoding the polypeptide of SEQ ID NO: 235 or fragments thereof. In some embodiments, the polypeptide of SEQ ID NO: 235, or a fragment thereof, is encoded by the polynucleotide of SEQ ID NO: 236 or fragments thereof.

The disclosure also provides an isolated polynucleotide encoding the polypeptide of SEQ ID NO: 237 or fragments thereof. In some embodiments, the polypeptide of SEQ ID NO: 237, or a fragment thereof, is encoded by the polynucleotide of SEQ ID NO: 238 or fragments thereof.

The disclosure also provides an isolated polynucleotide encoding the polypeptide of SEQ ID NO: 239 or fragments thereof. In some embodiments, the polypeptide of SEQ ID NO: 239, or a fragment thereof, is encoded by the polynucleotide of SEQ ID NO: 240 or fragments thereof.

The disclosure also provides an isolated polynucleotide encoding the polypeptide of SEQ ID NO: 241 or fragments thereof. In some embodiments, the polypeptide of SEQ ID NO: 241, or a fragment thereof, is encoded by the polynucleotide of SEQ ID NO: 242 or fragments thereof.

The disclosure also provides an isolated polynucleotide encoding the polypeptide of SEQ ID NO: 243 or fragments thereof. In some embodiments, the polypeptide of SEQ ID NO: 243, or a fragment thereof, is encoded by the polynucleotide of SEQ ID NO: 244 or fragments thereof.

The disclosure also provides an isolated polynucleotide encoding the polypeptide of SEQ ID NO: 245 or fragments thereof. In some embodiments, the polypeptide of SEQ ID NO: 245, or a fragment thereof, is encoded by the polynucleotide of SEQ ID NO: 246 or fragments thereof.

The disclosure also provides an isolated polynucleotide encoding the polypeptide of SEQ ID NO: 247 or fragments thereof. In some embodiments, the polypeptide of SEQ ID NO: 247, or a fragment thereof, is encoded by the polynucleotide of SEQ ID NO: 248 or fragments thereof.

The disclosure also provides an isolated polynucleotide encoding the polypeptide of SEQ ID NO: 249 or fragments thereof. In some embodiments, the polypeptide of SEQ ID NO: 249, or a fragment thereof, is encoded by the polynucleotide of SEQ ID NO: 250 or fragments thereof.

The disclosure also provides an isolated polynucleotide encoding the polypeptide of SEQ ID NO: 251 or fragments thereof. In some embodiments, the polypeptide of SEQ ID NO: 251, or a fragment thereof, is encoded by the polynucleotide of SEQ ID NO: 252 or fragments thereof.

The disclosure also provides an isolated polynucleotide encoding the polypeptide of SEQ ID NO: 253 or fragments thereof. In some embodiments, the polypeptide of SEQ ID NO: 253, or a fragment thereof, is encoded by the polynucleotide of SEQ ID NO: 254 or fragments thereof.

The disclosure also provides an isolated polynucleotide encoding the polypeptide of SEQ ID NO: 255 or fragments thereof. In some embodiments, the polypeptide of SEQ ID NO: 255, or a fragment thereof, is encoded by the polynucleotide of SEQ ID NO: 256 or fragments thereof.

The disclosure also provides an isolated polynucleotide encoding the polypeptide of SEQ ID NO: 257 or fragments thereof. In some embodiments, the polypeptide of SEQ ID NO: 257, or a fragment thereof, is encoded by the polynucleotide of SEQ ID NO: 258 or fragments thereof.

The disclosure also provides an isolated polynucleotide encoding the polypeptide of SEQ ID NO: 259 or fragments thereof. In some embodiments, the polypeptide of SEQ ID NO: 259, or a fragment thereof, is encoded by the polynucleotide of SEQ ID NO: 260 or fragments thereof.

The disclosure also provides an isolated polynucleotide encoding the polypeptide of SEQ ID NO: 261 or fragments thereof. In some embodiments, the polypeptide of SEQ ID NO: 261, or a fragment thereof, is encoded by the polynucleotide of SEQ ID NO: 262 or fragments thereof.

The disclosure also provides an isolated polynucleotide encoding the polypeptide of SEQ ID NO: 263 or fragments thereof. In some embodiments, the polypeptide of SEQ ID NO: 263, or a fragment thereof, is encoded by the polynucleotide of SEQ ID NO: 264 or fragments thereof.

The disclosure also provides an isolated polynucleotide encoding the polypeptide of SEQ ID NO: 265 or fragments thereof. In some embodiments, the polypeptide of SEQ ID NO: 265, or a fragment thereof, is encoded by the polynucleotide of SEQ ID NO: 266 or fragments thereof.

The disclosure also provides an isolated polynucleotide encoding the polypeptide of SEQ ID NO: 267 or fragments thereof. In some embodiments, the polypeptide of SEQ ID NO: 267, or a fragment thereof, is encoded by the polynucleotide of SEQ ID NO: 268 or fragments thereof.

The disclosure also provides an isolated polynucleotide encoding the polypeptide of SEQ ID NO: 269 or fragments thereof. In some embodiments, the polypeptide of SEQ ID NO: 269, or a fragment thereof, is encoded by the polynucleotide of SEQ ID NO: 270 or fragments thereof.

The disclosure also provides an isolated polynucleotide encoding the polypeptide of SEQ ID NO: 271 or fragments thereof. In some embodiments, the polypeptide of SEQ ID NO: 271, or a fragment thereof, is encoded by the polynucleotide of SEQ ID NO: 272 or fragments thereof.

The disclosure also provides an isolated polynucleotide encoding the polypeptide of SEQ ID NO: 273 or fragments thereof. In some embodiments, the polypeptide of SEQ ID NO: 273, or a fragment thereof, is encoded by the polynucleotide of SEQ ID NO: 274 or fragments thereof.

The disclosure also provides an isolated polynucleotide encoding the polypeptide of SEQ ID NO: 275 or fragments thereof. In some embodiments, the polypeptide of SEQ ID NO: 275, or a fragment thereof, is encoded by the polynucleotide of SEQ ID NO: 276 or fragments thereof.

The disclosure also provides an isolated polynucleotide encoding the polypeptide of SEQ ID NO: 277 or fragments thereof. In some embodiments, the polypeptide of SEQ ID NO: 277, or a fragment thereof, is encoded by the polynucleotide of SEQ ID NO: 278 or fragments thereof.

The disclosure also provides an isolated polynucleotide encoding the polypeptide of SEQ ID NO: 279 or fragments thereof. In some embodiments, the polypeptide of SEQ ID NO: 279, or a fragment thereof, is encoded by the polynucleotide of SEQ ID NO: 280 or fragments thereof.

The disclosure also provides an isolated polynucleotide encoding the polypeptide of SEQ ID NO: 281 or fragments thereof. In some embodiments, the polypeptide of SEQ ID NO: 281, or a fragment thereof, is encoded by the polynucleotide of SEQ ID NO: 282 or fragments thereof.

The disclosure also provides an isolated polynucleotide encoding the polypeptide of SEQ ID NO: 283 or fragments thereof. In some embodiments, the polypeptide of SEQ ID NO: 283, or a fragment thereof, is encoded by the polynucleotide of SEQ ID NO: 284 or fragments thereof.

The disclosure also provides an isolated polynucleotide encoding the polypeptide of SEQ ID NO: 285 or fragments thereof. In some embodiments, the polypeptide of SEQ ID NO: 285, or a fragment thereof, is encoded by the polynucleotide of SEQ ID NO: 286 or fragments thereof.

The disclosure also provides an isolated polynucleotide encoding the polypeptide of SEQ ID NO: 287 or fragments thereof. In some embodiments, the polypeptide of SEQ ID NO: 287, or a fragment thereof, is encoded by the polynucleotide of SEQ ID NO: 288 or fragments thereof.

The disclosure also provides an isolated polynucleotide encoding the polypeptide of SEQ ID NO: 289 or fragments thereof. In some embodiments, the polypeptide of SEQ ID NO: 289, or a fragment thereof, is encoded by the polynucleotide of SEQ ID NO: 290 or fragments thereof.

The disclosure also provides an isolated polynucleotide encoding the polypeptide of SEQ ID NO: 291 or fragments thereof. In some embodiments, the polypeptide of SEQ ID NO: 291, or a fragment thereof, is encoded by the polynucleotide of SEQ ID NO: 292 or fragments thereof.

The disclosure also provides an isolated polynucleotide encoding the polypeptide of SEQ ID NO: 293 or fragments thereof. In some embodiments, the polypeptide of SEQ ID NO: 293, or a fragment thereof, is encoded by the polynucleotide of SEQ ID NO: 294 or fragments thereof.

The disclosure also provides an isolated polynucleotide encoding the polypeptide of SEQ ID NO: 295 or fragments thereof. In some embodiments, the polypeptide of SEQ ID NO: 295, or a fragment thereof, is encoded by the polynucleotide of SEQ ID NO: 296 or fragments thereof.

The disclosure also provides an isolated polynucleotide encoding the polypeptide of SEQ ID NO: 297 or fragments thereof. In some embodiments, the polypeptide of SEQ ID NO: 297, or a fragment thereof, is encoded by the polynucleotide of SEQ ID NO: 298 or fragments thereof.

The disclosure also provides an isolated polynucleotide encoding the polypeptide of SEQ ID NO: 299 or fragments thereof. In some embodiments, the polypeptide of SEQ ID NO: 299, or a fragment thereof, is encoded by the polynucleotide of SEQ ID NO: 300 or fragments thereof.

The disclosure also provides an isolated polynucleotide encoding the polypeptide of SEQ ID NO: 301 or fragments thereof. In some embodiments, the polypeptide of SEQ ID NO: 301, or a fragment thereof, is encoded by the polynucleotide of SEQ ID NO: 302 or fragments thereof.

The disclosure also provides an isolated polynucleotide encoding the polypeptide of SEQ ID NO: 303 or fragments thereof. In some embodiments, the polypeptide of SEQ ID NO: 303, or a fragment thereof, is encoded by the polynucleotide of SEQ ID NO: 304 or fragments thereof.

The disclosure also provides an isolated polynucleotide encoding the polypeptide of SEQ ID NO: 305 or fragments thereof. In some embodiments, the polypeptide of SEQ ID NO: 305, or a fragment thereof, is encoded by the polynucleotide of SEQ ID NO: 306 or fragments thereof.

The disclosure also provides an isolated polynucleotide encoding the polypeptide of SEQ ID NO: 307 or fragments thereof. In some embodiments, the polypeptide of SEQ ID NO: 307, or a fragment thereof, is encoded by the polynucleotide of SEQ ID NO: 308 or fragments thereof.

The disclosure also provides an isolated polynucleotide encoding the polypeptide of SEQ ID NO: 309 or fragments thereof. In some embodiments, the polypeptide of SEQ ID NO: 309, or a fragment thereof, is encoded by the polynucleotide of SEQ ID NO: 310 or fragments thereof.

The disclosure also provides an isolated polynucleotide encoding the polypeptide of SEQ ID NO: 311 or fragments thereof. In some embodiments, the polypeptide of SEQ ID NO: 311, or a fragment thereof, is encoded by the polynucleotide of SEQ ID NO: 312 or fragments thereof.

The disclosure also provides an isolated polynucleotide encoding the polypeptide of SEQ ID NO: 313 or fragments thereof. In some embodiments, the polypeptide of SEQ ID NO: 313, or a fragment thereof, is encoded by the polynucleotide of SEQ ID NO: 314 or fragments thereof.

The disclosure also provides an isolated polynucleotide encoding the polypeptide of SEQ ID NO: 315 or fragments thereof. In some embodiments, the polypeptide of SEQ ID NO: 315, or a fragment thereof, is encoded by the polynucleotide of SEQ ID NO: 316 or fragments thereof.

The disclosure also provides an isolated polynucleotide encoding the polypeptide of SEQ ID NO: 317 or fragments thereof. In some embodiments, the polypeptide of SEQ ID NO: 317, or a fragment thereof, is encoded by the polynucleotide of SEQ ID NO: 318 or fragments thereof.

The disclosure also provides an isolated polynucleotide encoding the polypeptide of SEQ ID NO: 319 or fragments thereof. In some embodiments, the polypeptide of SEQ ID NO: 319, or a fragment thereof, is encoded by the polynucleotide of SEQ ID NO: 320 or fragments thereof.

The disclosure also provides an isolated polynucleotide encoding the polypeptide of SEQ ID NO: 321 or fragments thereof. In some embodiments, the polypeptide of SEQ ID NO: 321, or a fragment thereof, is encoded by the polynucleotide of SEQ ID NO: 322 or fragments thereof.

The disclosure also provides an isolated polynucleotide encoding the polypeptide of SEQ ID NO: 323 or fragments thereof. In some embodiments, the polypeptide of SEQ ID NO: 323, or a fragment thereof, is encoded by the polynucleotide of SEQ ID NO: 324 or fragments thereof.

The disclosure also provides an isolated polynucleotide encoding the polypeptide of SEQ ID NO: 325 or fragments thereof. In some embodiments, the polypeptide of SEQ ID NO: 325, or a fragment thereof, is encoded by the polynucleotide of SEQ ID NO: 326 or fragments thereof.

The disclosure also provides an isolated polynucleotide encoding the polypeptide of SEQ ID NO: 327 or fragments thereof. In some embodiments, the polypeptide of SEQ ID NO: 327, or a fragment thereof, is encoded by the polynucleotide of SEQ ID NO: 328 or fragments thereof.

The disclosure also provides an isolated polynucleotide encoding the polypeptide of SEQ ID NO: 329 or fragments thereof. In some embodiments, the polypeptide of SEQ ID NO: 329, or a fragment thereof, is encoded by the polynucleotide of SEQ ID NO: 330 or fragments thereof.

The disclosure also provides an isolated polynucleotide encoding the polypeptide of SEQ ID NO: 331 or fragments thereof. In some embodiments, the polypeptide of SEQ ID NO: 331, or a fragment thereof, is encoded by the polynucleotide of SEQ ID NO: 332 or fragments thereof.

The disclosure also provides an isolated polynucleotide encoding the polypeptide of SEQ ID NO: 333 or fragments thereof. In some embodiments, the polypeptide of SEQ ID NO: 333, or a fragment thereof, is encoded by the polynucleotide of SEQ ID NO: 334 or fragments thereof.

The disclosure also provides an isolated polynucleotide encoding the polypeptide of SEQ ID NO: 335 or fragments thereof. In some embodiments, the polypeptide of SEQ ID NO: 335, or a fragment thereof, is encoded by the polynucleotide of SEQ ID NO: 336 or fragments thereof.

The disclosure also provides an isolated polynucleotide encoding the polypeptide of SEQ ID NO: 337 or fragments thereof. In some embodiments, the polypeptide of SEQ ID NO: 337, or a fragment thereof, is encoded by the polynucleotide of SEQ ID NO: 338 or fragments thereof.

The disclosure also provides an isolated polynucleotide encoding the polypeptide of SEQ ID NO: 339 or fragments thereof. In some embodiments, the polypeptide of SEQ ID NO: 339, or a fragment thereof, is encoded by the polynucleotide of SEQ ID NO: 340 or fragments thereof.

The disclosure also provides an isolated polynucleotide encoding the polypeptide of SEQ ID NO: 341 or fragments thereof. In some embodiments, the polypeptide of SEQ ID NO: 341, or a fragment thereof, is encoded by the polynucleotide of SEQ ID NO: 342 or fragments thereof.

The disclosure also provides an isolated polynucleotide encoding the polypeptide of SEQ ID NO: 343 or fragments thereof. In some embodiments, the polypeptide of SEQ ID NO: 343, or a fragment thereof, is encoded by the polynucleotide of SEQ ID NO: 344 or fragments thereof.

The disclosure also provides an isolated polynucleotide encoding the polypeptide of SEQ ID NO: 345 or fragments thereof. In some embodiments, the polypeptide of SEQ ID NO: 345, or a fragment thereof, is encoded by the polynucleotide of SEQ ID NO: 346 or fragments thereof.

The disclosure also provides an isolated polynucleotide encoding the polypeptide of SEQ ID NO: 347 or fragments thereof. In some embodiments, the polypeptide of SEQ ID NO: 347, or a fragment thereof, is encoded by the polynucleotide of SEQ ID NO: 348 or fragments thereof.

The disclosure also provides an isolated polynucleotide encoding the polypeptide of SEQ ID NO: 349 or fragments thereof. In some embodiments, the polypeptide of SEQ ID NO: 349, or a fragment thereof, is encoded by the polynucleotide of SEQ ID NO: 350 or fragments thereof.

The disclosure also provides an isolated polynucleotide encoding the polypeptide of SEQ ID NO: 351 or fragments thereof. In some embodiments, the polypeptide of SEQ ID NO: 351, or a fragment thereof, is encoded by the polynucleotide of SEQ ID NO: 352 or fragments thereof.

The disclosure also provides an isolated polynucleotide encoding the polypeptide of SEQ ID NO: 353 or fragments thereof. In some embodiments, the polypeptide of SEQ ID NO: 353, or a fragment thereof, is encoded by the polynucleotide of SEQ ID NO: 354 or fragments thereof.

The disclosure also provides an isolated polynucleotide encoding the polypeptide of SEQ ID NO: 355 or fragments thereof. In some embodiments, the polypeptide of SEQ ID NO: 355, or a fragment thereof, is encoded by the polynucleotide of SEQ ID NO: 356 or fragments thereof.

The disclosure also provides an isolated polynucleotide encoding the polypeptide of SEQ ID NO: 357 or fragments thereof. In some embodiments, the polypeptide of SEQ ID NO: 357, or a fragment thereof, is encoded by the polynucleotide of SEQ ID NO: 358 or fragments thereof.

The disclosure also provides an isolated polynucleotide encoding the polypeptide of SEQ ID NO: 359 or fragments thereof. In some embodiments, the polypeptide of SEQ ID NO: 359, or a fragment thereof, is encoded by the polynucleotide of SEQ ID NO: 360 or fragments thereof.

The disclosure also provides an isolated polynucleotide encoding the polypeptide of SEQ ID NO: 361 or fragments thereof. In some embodiments, the polypeptide of SEQ ID NO: 361, or a fragment thereof, is encoded by the polynucleotide of SEQ ID NO: 362 or fragments thereof.

The disclosure also provides an isolated polynucleotide encoding the polypeptide of SEQ ID NO: 363 or fragments thereof. In some embodiments, the polypeptide of SEQ ID NO: 363, or a fragment thereof, is encoded by the polynucleotide of SEQ ID NO: 364 or fragments thereof.

The disclosure also provides an isolated polynucleotide encoding the polypeptide of SEQ ID NO: 365 or fragments thereof. In some embodiments, the polypeptide of SEQ ID NO: 365, or a fragment thereof, is encoded by the polynucleotide of SEQ ID NO: 366 or fragments thereof.

The disclosure also provides an isolated polynucleotide encoding the polypeptide of SEQ ID NO: 367 or fragments thereof. In some embodiments, the polypeptide of SEQ ID NO: 367, or a fragment thereof, is encoded by the polynucleotide of SEQ ID NO: 368 or fragments thereof.

The disclosure also provides an isolated polynucleotide encoding the polypeptide of SEQ ID NO: 369 or fragments thereof. In some embodiments, the polypeptide of SEQ ID NO: 369, or a fragment thereof, is encoded by the polynucleotide of SEQ ID NO: 370 or fragments thereof.

The disclosure also provides an isolated polynucleotide encoding the polypeptide of SEQ ID NO: 371 or fragments thereof. In some embodiments, the polypeptide of SEQ ID NO: 371, or a fragment thereof, is encoded by the polynucleotide of SEQ ID NO: 372 or fragments thereof.

The disclosure also provides an isolated polynucleotide encoding the polypeptide of SEQ ID NO: 373 or fragments thereof. In some embodiments, the polypeptide of SEQ ID NO: 373, or a fragment thereof, is encoded by the polynucleotide of SEQ ID NO: 374 or fragments thereof.

The disclosure also provides an isolated polynucleotide encoding the polypeptide of SEQ ID NO: 375 or fragments thereof. In some embodiments, the polypeptide of SEQ ID NO: 375, or a fragment thereof, is encoded by the polynucleotide of SEQ ID NO: 376 or fragments thereof.

The disclosure also provides an isolated polynucleotide encoding the polypeptide of SEQ ID NO: 377 or fragments thereof. In some embodiments, the polypeptide of SEQ ID NO: 377, or a fragment thereof, is encoded by the polynucleotide of SEQ ID NO: 378 or fragments thereof.

The disclosure also provides an isolated polynucleotide encoding the polypeptide of SEQ ID NO: 379 or fragments thereof. In some embodiments, the polypeptide of SEQ ID NO: 379, or a fragment thereof, is encoded by the polynucleotide of SEQ ID NO: 380 or fragments thereof.

The disclosure also provides an isolated polynucleotide encoding the polypeptide of SEQ ID NO: 381 or fragments thereof. In some embodiments, the polypeptide of SEQ ID NO: 381, or a fragment thereof, is encoded by the polynucleotide of SEQ ID NO: 382 or fragments thereof.

The disclosure also provides an isolated polynucleotide encoding the polypeptide of SEQ ID NO: 383 or fragments thereof. In some embodiments, the polypeptide of SEQ ID NO: 383, or a fragment thereof, is encoded by the polynucleotide of SEQ ID NO: 384 or fragments thereof.

The disclosure also provides an isolated polynucleotide encoding the polypeptide of SEQ ID NO: 385 or fragments thereof. In some embodiments, the polypeptide of SEQ ID NO: 385, or a fragment thereof, is encoded by the polynucleotide of SEQ ID NO: 386 or fragments thereof.

The disclosure also provides an isolated polynucleotide encoding the polypeptide of SEQ ID NO: 387 or fragments thereof. In some embodiments, the polypeptide of SEQ ID NO: 387, or a fragment thereof, is encoded by the polynucleotide of SEQ ID NO: 388 or fragments thereof.

The disclosure also provides an isolated polynucleotide encoding the polypeptide of SEQ ID NO: 389 or fragments thereof. In some embodiments, the polypeptide of SEQ ID NO: 389, or a fragment thereof, is encoded by the polynucleotide of SEQ ID NO: 390 or fragments thereof.

The disclosure also provides an isolated polynucleotide encoding the polypeptide of SEQ ID NO: 391 or fragments thereof. In some embodiments, the polypeptide of SEQ ID NO: 391, or a fragment thereof, is encoded by the polynucleotide of SEQ ID NO: 392 or fragments thereof.

The disclosure also provides an isolated polynucleotide encoding the polypeptide of SEQ ID NO: 393 or fragments thereof. In some embodiments, the polypeptide of SEQ ID NO: 393, or a fragment thereof, is encoded by the polynucleotide of SEQ ID NO: 394 or fragments thereof.

The disclosure also provides an isolated polynucleotide encoding the polypeptide of SEQ ID NO: 395 or fragments thereof. In some embodiments, the polypeptide of SEQ ID NO: 395, or a fragment thereof, is encoded by the polynucleotide of SEQ ID NO: 396 or fragments thereof.

The disclosure also provides an isolated polynucleotide encoding the polypeptide of SEQ ID NO: 397 or fragments thereof. In some embodiments, the polypeptide of SEQ ID NO: 397, or a fragment thereof, is encoded by the polynucleotide of SEQ ID NO: 398 or fragments thereof.

The disclosure also provides an isolated polynucleotide encoding the polypeptide of SEQ ID NO: 399 or fragments thereof. In some embodiments, the polypeptide of SEQ ID NO: 399, or a fragment thereof, is encoded by the polynucleotide of SEQ ID NO: 400 or fragments thereof.

The disclosure also provides an isolated polynucleotide encoding the polypeptide of SEQ ID NO: 401 or fragments thereof. In some embodiments, the polypeptide of SEQ ID NO: 401, or a fragment thereof, is encoded by the polynucleotide of SEQ ID NO: 402 or fragments thereof.

The disclosure also provides an isolated polynucleotide encoding the polypeptide of SEQ ID NO: 403 or fragments thereof. In some embodiments, the polypeptide of SEQ ID NO: 403, or a fragment thereof, is encoded by the polynucleotide of SEQ ID NO: 404 or fragments thereof.

The disclosure also provides an isolated polynucleotide encoding the polypeptide of SEQ ID NO: 405 or fragments thereof. In some embodiments, the polypeptide of SEQ ID NO: 405, or a fragment thereof, is encoded by the polynucleotide of SEQ ID NO: 406 or fragments thereof.

The disclosure also provides an isolated polynucleotide encoding the polypeptide of SEQ ID NO: 407 or fragments thereof. In some embodiments, the polypeptide of SEQ ID NO: 407, or a fragment thereof, is encoded by the polynucleotide of SEQ ID NO: 408 or fragments thereof.

The disclosure also provides an isolated polynucleotide encoding the polypeptide of SEQ ID NO: 387 or fragments thereof. In some embodiments, the polypeptide of SEQ ID NO: 421, or a fragment thereof, is encoded by the polynucleotide of SEQ ID NO: 422 or fragments thereof.

In some embodiments, the heterologous polynucleotide is an in-frame heterologous polynucleotide.

For expression in various hosts, the polynucleotides may be codon-optimized utilizing known methods.

In some embodiments, the isolated heterologous polynucleotide is an in-frame heterologous polynucleotide.

In some embodiments, the polynucleotide comprises DNA or RNA.

In some embodiments, the polynucleotide comprises RNA.

In some embodiments, RNA is mRNA.

### Variants of and Engineered Polynucleotides, Polypeptides, Heterologous Polynucleotides and Heterologous Polypeptides of the Disclosure

Variants of the polynucleotides, polypeptides, heterologous polynucleotides and heterologous polypeptides or fragments thereof are within the scope of the disclosure. For example, variants may comprise one or more substitutions, deletions or insertions, as long as the variants retain or have improved characteristics (such as immunogenicity or stability) when compared to the parent. In some embodiments, the sequence identity may be about 80%, 81%, 82%, 83%, 84%, 85%, 86%, 87%, 88%, 89%, 90%, 91%, 92%, 93%, 94%, 95%, 96%, 97%, 98% or 99% between the parent and the variant. In some embodiments, variants are generated by conservative substitutions.

In some embodiments, the identity is about 80%. In some embodiments, the identity is about 85%. In some embodiments, the identity is about 90%. In some embodiments, the identity is about 91%. In some embodiments, the identity is about 91%. In some embodiments, the identity is about 92%. In some embodiments, the identity is about 93%. In some embodiments, the identity is about 94%. In some embodiments, the identity is 94%. In some embodiments, the identity is about 95%. In some embodiments, the identity is about 96%. In some embodiments, the identity is about 97%. In some embodiments, the identity is about 98%. In some embodiments, the identity is about 99%.

The percent identity between the two sequences is a function of the number of identical positions shared by the sequences (i.e., % identity=number of identical positions/total number of positions x100), taking into account the number of gaps, and the length of each gap, which need to be introduced for optimal alignment of the two sequences. The percent identity between two amino acid sequences may be determined using the algorithm of E. Meyers and W. Miller (Comput Appl Biosci 4:11-17 (1988)) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch (J Mol Biol 48:444-453 (1970)) algorithm which has been incorporated into the GAP program in the GCG software package (available at http_//_www_gcg_com), using either a Blossum 62 matrix or a PAM250 matrix, and a gap weight of 16, 14, 12, 10, 8, 6, or 4 and a length weight of 1, 2, 3, 4, 5, or 6.

The variants of the polypeptides or the heterologous polypeptides or fragments thereof containing one amino acid alteration generally retain similar tertiary structure and antigenicity relative to the parent. In some instances, the variant may also contain at least one amino acid alteration that causes the variant to have increased antigenicity, increased binding affinity to TCR or to antibody, or both. The variants of the polypeptides or the heterologous polypeptides may also have improved ability to bind to a HLA molecule.

The variants of the disclosure may be engineered to contain conservative substitutions. Conservative substitutions are herein defined as exchanges within one of the following five groups: Group 1-small aliphatic, nonpolar or slightly polar residues (Ala, Ser, Thr, Pro, Gly); Group 2-polar, negatively charged residues and their amides (Asp, Asn, Glu, Gin); Group 3-polar, positively charged residues (His, Arg, Lys); Group 4-large, aliphatic, nonpolar residues (Met, Leu, lie, Val, Cys); and Group 5-large, aromatic residues (Phe, Tyr, Trp).

The variants of the disclosure may be engineered to contain less conservative substitutions, such as the replacement of one amino acid by another that has similar characteristics but is somewhat different in size, such as replacement of an alanine by an isoleucine residue. The variants of the disclosure may also be engineered to contain highly non-conservative substitutions which may involve substituting an acidic amino acid for one that is polar, or even for one that is basic in character.

Additional substitutions that may be made to generate variants of the disclosure include substitutions may involve structures other than the common L-amino acids. Thus, D-amino and non-standard amino acids (i.e., other than the common naturally occurring proteinogenic amino acids) may also be used for substitution purposes to produce variants with enhanced immunogenicity when compared to the parent.

If substitutions at more than one position are found to result in polypeptides or heterologous polypeptides with substantially equivalent or greater immunogenicity, then combinations of those substitutions may be tested to determine if the combined substitutions result in additive or synergistic effects on the immunogenicity of the variant.

The amino acid residues that do not substantially contribute to interactions with the TCR may be modified by replacement with other amino acid whose incorporation does not substantially affect T-cell reactivity and does not eliminate binding to the relevant MHC. The amino acid residues that do not substantially contribute to interactions with the TCR may also be deleted as long as the deletion does not substantially affect T-cell reactivity and does not eliminate binding to the relevant MHC.

In addition, the polypeptides or the heterologous polypeptides or fragments thereof or variants may be further modified to improve stability and/or binding to MHC molecules in order to elicit a stronger immune response. Methods for such an optimization of a peptide sequence are well known in the art and include, for example, the introduction of reverse peptide bonds or non-peptide bonds. In a reverse peptide bond amino acid residues are not joined by peptide (—CO—NH—) linkages but the peptide bond is reversed. Such retro-inverso peptidomimetics may be made using methods known in the art, for example such as those described in Meziere et al (1997) (Meziere et al., 1997). This approach involves making pseudopeptides containing changes involving the backbone, and not the orientation of side chains. Meziere et al. (Meziere et al., 1997) show that for MHC binding and T helper cell responses, these pseudopeptides are useful. Retro-inverse peptides, which contain NH—CO bonds instead of CO—NH peptide bonds, are much more resistant to proteolysis. Additional non-peptide bond that may be used are, for example, —CH₂—NH, —CH₂S—, —CH₂CH₂—, —CH═CH—, —COCH₂—, —CH(OH)CH₂—, and —CH₂SO—.

The polypeptides or the heterologous polypeptides or fragments thereof, or variants of the disclosure may be synthesized with additional chemical groups present at their amino and/or carboxy termini, to enhance the stability, bioavailability, and/or affinity of the peptides. For example, hydrophobic groups such as carbobenzoxyl, dansyl, or t-butyloxycarbonyl groups may be added to the amino terminus. Likewise, an acetyl group or a 9-fluorenylmethoxy-carbonyl group may be placed at the amino termini. Additionally, the hydrophobic group, t-butyloxycarbonyl, or an amido group may be added to the carboxy termini.

Further, the polypeptides or the heterologous polypeptides or fragments thereof, or variants of the disclosure may be synthesized to alter their steric configuration. For example, the D-isomer of one or more of the amino acid residues of the peptide may be used, rather than the usual L-isomer.

Similarly, the polypeptides or the heterologous polypeptides or fragments thereof, or variants of the disclosure may be modified chemically by reacting specific amino acids either before or after synthesis of the polypeptides or the heterologous polypeptides or fragments thereof, or variants of the disclosure. Examples for such modifications are well known in the art and are summarized e.g. in R. Lundblad, Chemical Reagents for Protein Modification, 3rd ed. CRC Press, 2004 (Lundblad, 2004), which is incorporated herein by reference. Chemical modification of amino acids includes but is not limited to, modification by acylation, amidination, pyridoxylation of lysine, reductive alkylation, trinitrobenzylation of amino groups with 2,4,6-trinitrobenzene sulphonic acid (TNBS), amide modification of carboxyl groups and sulphydryl modification by performic acid oxidation of cysteine to cysteic acid, formation of mercurial derivatives, formation of mixed disulphides with other thiol compounds, reaction with maleimide, carboxymethylation with iodoacetic acid or iodoacetamide and carbamoylation with cyanate at alkaline pH, although without limitation thereto. In this regard, the skilled person is referred to Chapter 15 of Current Protocols In Protein Science, Eds. Coligan et al. (John Wiley and Sons NY 1995-2000) (Coligan et a 1., 1995) for more extensive methodology relating to chemical modification of proteins.

Briefly, modification of e.g. arginyl residues in proteins is often based on the reaction of vicinal dicarbonyl compounds such as phenylglyoxal, 2,3-butanedione, and 1,2-cyclohexanedione to form an adduct. Another example is the reaction of methylglyoxal with arginine residues. Cysteine can be modified without concomitant modification of other nucleophilic sites such as lysine and histidine. As a result, a large number of reagents are available for the modification of cysteine. The websites of companies such as Sigma-Aldrich (http://www.sigma-aldrich.com) provide information on specific reagents. Selective reduction of disulfide bonds in proteins is also common. Disulfide bonds can be formed and oxidized during the heat treatment of biopharmaceuticals. Woodward's Reagent K may be used to modify specific glutamic acid residues. N-(3-(dimethylamino)propyl)-N′-ethylcarbodiimide can be used to form intra-molecular crosslinks between a lysine residue and a glutamic acid residue. For example, diethylpyrocarbonate is a reagent for the modification of histidyl residues in proteins. Histidine can also be modified using 4-hydroxy-2-nonenal. The reaction of lysine residues and other a-amino groups is, for example, useful in binding of peptides to surfaces or the cross-linking of proteins/peptides. Lysine is the site of attachment of poly(ethylene)glycol and the major site of modification in the glycosylation of proteins. Methionine residues in proteins can be modified with e.g. iodoacetamide, bromoethylamine, and chloramine T. Tetranitromethane and N-acetylimidazole can be used for the modification of tyrosyl residues. Cross-linking via the formation of dityrosine can be accomplished with hydrogen peroxide/copper ions. Recent studies on the modification of tryptophan have used N-bromosuccinimide, 2-hydroxy-5-nitrobenzyl bromide or 3-bromo-3-methyl-2-(2-nitrophenylmercapto)-3H-indole (BPNS-skatole). Successful modification of therapeutic proteins and peptides with PEG is often associated with an extension of circulatory half-life while cross-linking of proteins with glutaraldehyde, polyethylene glycol diacrylate and formaldehyde is used for the preparation of hydrogels. Chemical modification of allergens for immunotherapy is often achieved by carbamylation with potassium cyanate.

The disclosure provides an isolated polypeptide that is about 80%, 81%, 82%, 83%, 84%, 85%, 86%, 87%, 88%, 89%, 90%, 91%, 92%, 93%, 94%, 95%, 96%, 97%, 98% or 99% identical to the polypeptide of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177,179,181,183,185,187,189,191,193,195,197,199,201,203,205,207,209,211,213,215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, or 421.

The disclosure also provides an isolated polynucleotide that is about 80%, 81%, 82%, 83%, 84%, 85%, 86%, 87%, 88%, 89%, 90%, 91%, 92%, 93%, 94%, 95%, 96%, 97%, 98% or 99% identical to the polynucleotide of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, or 422.

The disclosure also provides an isolated polypeptide comprising an amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, or 421, and fragments thereof, wherein the polypeptide comprises one or more reverse peptide bonds.

In some embodiments, the reverse peptide bond comprises NH—CO bond.

In some embodiments, the reverse peptide bond comprises CH₂—NH, —CH₂S—, —CH₂CH₂—, —COCH₂—, —CH(OH)CH₂—, or —CH₂SO— bond.

The disclosure also provides an isolated polypeptide comprising an amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, or 421 wherein the polypeptide comprises one or more chemical modifications.

In some embodiments, the one or more chemical modification comprises modification with carbobenzoxyl, dansyl, t-butyloxycarbonyl, 9-fluorenylmethoxy-carbonyl or D-isomer of an amino acid.

### Methods of Making Polynucleotides and Polypeptides of the Disclosure

The polynucleotides of the disclosure or variants may be in the form of RNA or in the form of DNA obtained by cloning or produced synthetically. The DNA may be double-stranded or single-stranded.

Methods of generating polynucleotides and heterologous polynucleotides of the disclosure or variants are known in the art and include chemical synthesis, enzymatic synthesis (e.g. in vitro transcription), enzymatic or chemical cleavage of a longer precursor, chemical synthesis of smaller fragments of the polynucleotides followed by ligation of the fragments or known PCR methods. The polynucleotide sequence to be synthesized may be designed with the appropriate codons for the desired amino acid sequence. In general, preferred codons may be selected for the intended host in which the sequence will be used for expression.

Methods of making polypeptides and heterologous polypeptides of the disclosure are known in the art and include standard molecular biology techniques for cloning and expression of the polypeptides and chemical synthesis of the polypeptides.

Peptides may be synthesized by the Fmoc-polyamide mode of solid-phase peptide synthesis as disclosed by Lukas et al. (Lukas et al., 1981) and by references as cited therein. Temporary N-amino group protection is afforded by the 9-fluorenylmethyloxycarbonyl (Fmoc) group. Repetitive cleavage of this highly base-labile protecting group is done using 20% piperidine in N, N-dimethylformamide. Side-chain functionalities may be protected as their butyl ethers (in the case of serine threonine and tyrosine), butyl esters (in the case of glutamic acid and aspartic acid), butyloxycarbonyl derivative (in the case of lysine and histidine), trityl derivative (in the case of cysteine) and 4-methoxy-2,3,6-trimethylbenzenesulphonyl derivative (in the case of arginine).

Where glutamine or asparagine are C-terminal residues, use is made of the 4,4′-dimethoxybenzhydryl group for protection of the side chain amido functionalities. The solid-phase support is based on a polydimethyl-acrylamide polymer constituted from the three monomers dimethylacrylamide (backbone-monomer), bisacryloylethylene diamine (cross linker) and acryloylsarcosine methyl ester (functionalizing agent). The peptide-to-resin cleavable linked agent used is the acid-labile 4-hydroxymethyl-phenoxyacetic acid derivative. All amino acid derivatives are added as their preformed symmetrical anhydride derivatives with the exception of asparagine and glutamine, which are added using a reversed N, N-dicyclohexyl-carbodiimide/1 hydroxybenzotriazole mediated coupling procedure. All coupling and deprotection reactions are monitored using ninhydrin, trinitrobenzene sulphonic acid or isotin test procedures. Upon completion of synthesis, peptides are cleaved from the resin support with concomitant removal of side-chain protecting groups by treatment with 95% trifluoroacetic acid containing a 50% scavenger mix. Scavengers commonly used include ethanedithiol, phenol, anisole and water, the exact choice depending on the constituent amino acids of the peptide being synthesized. Also a combination of solid phase and solution phase methodologies for the synthesis of peptides is possible (see, for example, (Bruckdorfer et al., 2004), and the references as cited therein).

U.S. Pat. No. 4,897,445 provides a method for the solid phase synthesis of non-peptide bonds (—CH₂—NH) in polypeptide chains which involves polypeptides synthesized by standard procedures and the non-peptide bond synthesized by reacting an amino aldehyde and an amino acid in the presence of NaCNBH₃.

### Vectors and Recombinant Viruses of the Disclosure

The disclosure also provides a vector comprising a polynucleotide or a heterologous polynucleotide of the disclosure. The disclosure also provides vectors comprising a polynucleotide encoding for one or more of the polypeptides disclosed herein.

The disclosure also provides a vector comprising a polynucleotide encoding one or more polypeptides of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, or 421, or fragments thereof.

The disclosure also provides a vector comprising one or more polynucleotides of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, or 422, or fragments thereof.

The disclosure also provides a vector comprising a polynucleotide encoding one or more polypeptides of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

The disclosure also provides a vector comprising one or more polynucleotides of SEQ ID NOs: 8, 10, 12, 14, 18, 22, 24, 26, 28, 32, 34, 36, 38, 40, 42, 44, 46, 48, 52, 54, 56, 60, 62, 64, 66, 70, 72, 76, 80, 82, 84, 86, 90, 92, 102, 104, 106, 108, 110, 112, 114, 120, 122, 124, 126, 132, 134, 136, 144, 146, 148, 150, 152, 158, 162, 164, 166, 172, 174, 180, 186, 188, 198, 200, 208, 243, 218, 222, 224, 226, 242, 248, 250, 260, 266, 268, 270, 282, 286, 288, 290, 294, 298, 300, 302, 304, 306, 308, 330, 332, 334, 336, 338, 340, 342, 344, 346, 350, 354, 356, 358, 360, 362, 364, 366, 368, 370, 372, 376, 378, 380, 382, 384, 386, or 422, or fragments thereof.

The disclosure also provides a vector comprising a heterologous polynucleotide encoding a heterologous polypeptide comprising two or more polypeptides selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, and 421, and fragments thereof.

The disclosure also provides a vector comprising a heterologous polynucleotide comprising two or more polynucleotides selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, and 422, and fragments thereof.

The disclosure also provides a vector comprising a heterologous polynucleotide encoding a heterologous polypeptide comprising 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, or 204 polypeptides selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, and 421, and fragments thereof.

The disclosure also provides a vector comprising a heterologous polynucleotide comprising 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, or 204 polynucleotides selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, and 422, and fragments thereof.

In some embodiments, the vector is an expression vector. The vector may be a vector intended for expression of the polynucleotide or the heterologous polynucleotide of the disclosure in any host, such as bacteria, yeast or a mammal. Suitable expression vectors are typically replicable in the host organisms either as episomes or as an integral part of the host chromosomal DNA. Commonly, expression vectors contain selection markers such as ampicillin-resistance, hygromycin-resistance, tetracycline resistance, kanamycin resistance or neomycin resistance to permit detection of those cells transformed or transduced with the desired DNA sequences. Exemplary vectors are plasmids, cosmids, phages, viral vectors, transposons or artificial chromosomes.

Suitable vectors are known; many are commercially available for generating recombinant constructs. The following vectors are provided by way of example. Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene), pSVK3, pBPV, pMSG and pSVL (Pharmacia). Transposon vectors: Sleeping Beauty transposon and PiggyBac transposon.

In some embodiments, the vector is a viral vector. The vectors of the disclosure may be utilized to generate recombinant viruses comprising the vectors of the disclosure or to express the polypeptides of the disclosure. Viral vectors are derived from naturally occurring virus genomes, which typically are modified to be replication incompetent, e.g. non-replicating. Non-replicating viruses require the provision of proteins in trans for replication. Typically, those proteins are stably or transiently expressed in a viral producer cell line, thereby allowing replication of the virus. The viral vectors are, thus, typically infectious and non-replicating. Viral vectors may be adenovirus vectors, adeno-associated virus (AAV) vectors (e.g., AAV type 5 and type 2), alphavirus vectors (e.g., Venezuelan equine encephalitis virus (VEE), Sindbis virus (SIN), Semliki forest virus (SFV), and VEE-SIN chimeras), herpes virus vectors (e.g. vectors derived from cytomegaloviruses, like rhesus cytomegalovirus (RhCMV)), arena virus vectors (e.g. lymphocytic choriomeningitis virus (LCMV) vectors), measles virus vectors, pox virus vectors (e.g., vaccinia virus, modified vaccinia virus Ankara (MVA), NYVAC (derived from the Copenhagen strain of vaccinia), and avipox vectors: canarypox (ALVAC) and fowlpox (FPV) vectors), vesicular stomatitis virus vectors, retrovirus vectors, lentivirus vectors, viral like particles, baculoviral vectors and bacterial spores. The vectors of the disclosure may be generated using known techniques.

### Adenovirus Vectors

In some embodiments, the viral vector is derived from an adenovirus. In some embodiments, the recombinant virus comprising the vector is derived from an adenovirus.

Adenovirus vectors may be derived from human adenovirus (Ad) but also from adenoviruses that infect other species, such as bovine adenovirus (e.g. bovine adenovirus 3, BAdV3), a canine adenovirus (e.g. CAdV2), a porcine adenovirus (e.g. PAdV3 or 5), or great apes, such as Chimpanzee (Pan), Gorilla (Gorilla), Orangutan (Pongo), Bonobo (Pan paniscus) and common chimpanzee (Pan troglodytes). Typically, naturally occurring great ape adenoviruses are isolated from stool samples of the respective great ape.

Human adenovirus vectors may be derived from various adenovirus serotypes, for example from human adenovirus serotypes hAd5, hAd7, hAd11, hAd26, hAd34, hAd35, hAd48, hAd49 or hAd50 (the serotypes are also referred to as Ad5, Ad7, Ad11, Ad26, Ad34, Ad35, Ad48, Ad49 or Ad50).

Great ape adenovirus vectors may be derived from various adenovirus serotypes, for example from great ape adenovirus serotypes GAd20, Gad19, GAd21, GAd25, GAd26, GAd27,

GAd28, GAd29, GAd30, GAd31, ChAd3, ChAd4, ChAd5, ChAd6, ChAd7, ChAd8, ChAd9, ChAd10, ChAd11, ChAd16, ChAdI7, ChAd19, ChAd20, ChAd22, ChAd24, ChAd26, ChAd30, ChAd31, ChAd37, ChAd38, ChAd44, ChAd55, ChAd63, ChAd73, ChAd82, ChAd83, ChAd146, ChAd147, PanAd1, PanAd2, or PanAd3.

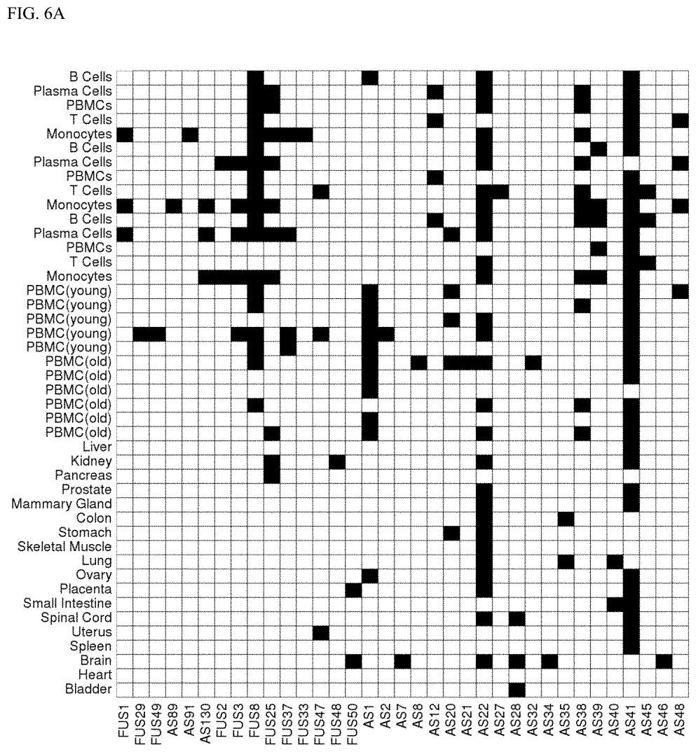

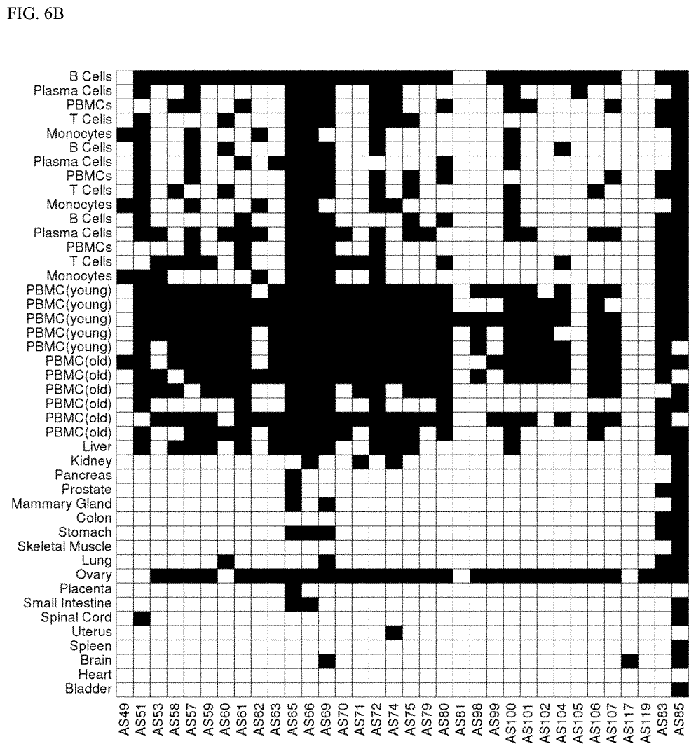

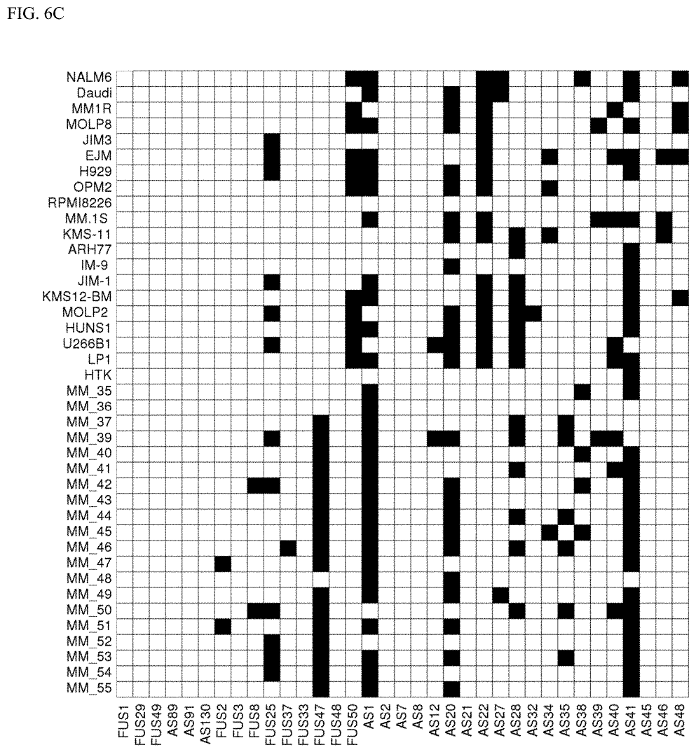

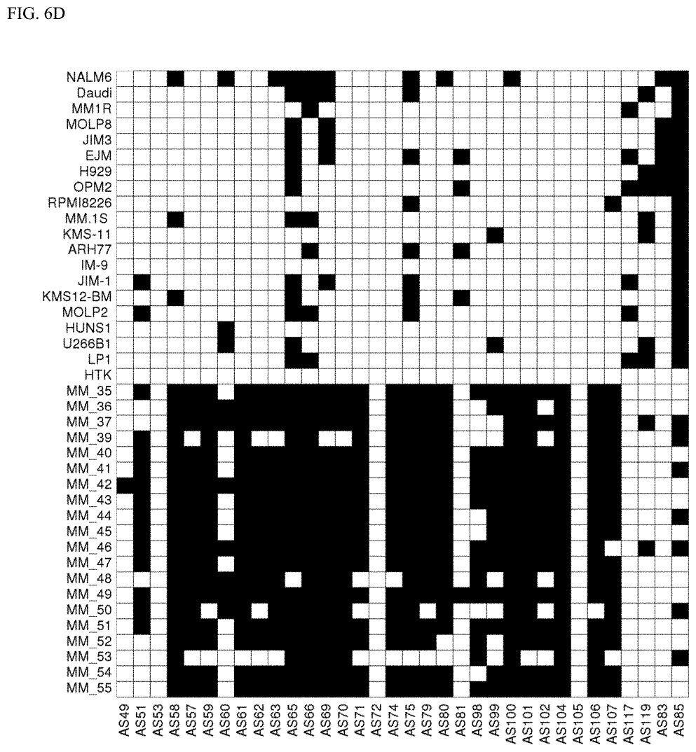

Adenovirus vectors are known in the art. The sequences of most of the human and non-human adenoviruses are known, and for others can be obtained using routine procedures. An exemplary genome sequence of Ad26 is found in GenBank Accession number EF153474 and in SEQ ID NO: 1 of Int. Pat. Publ. No. WO2007/104792. An exemplary genome sequence of Ad35 is found in FIG. 6 of Int. Pat. Publ. No. WO2000/70071. Vectors based on Ad26 are described for example, in Int. Pat. Publ. No. WO2007/104792. Vectors based on Ad35 are described for example in U.S. Pat. No. 7,270,811 and Int. Pat. Publ. No. WO2000/70071. Vectors based on ChAd3, ChAd4, ChAd5, ChAd6, ChAd7, ChAd8, ChAd9, ChAd10, ChAd11, ChAd16, ChAd17, ChAd19, ChAd20, ChAd22, ChAd24, ChAd26, ChAd30, ChAd31, ChAd37, ChAd38, ChAd44, ChAd63 and ChAd82 are described in WO2005/071093. Vectors based on PanAd1, PanAd2, PanAd3, ChAd55, ChAd73, ChAd83, ChAd146, and ChAd147 are described in Int. Pat. Publ. No. WO2010/086189.

Adenovirus vectors are engineered to comprise at least one functional deletion or a complete removal of a gene product that is essential for viral replication, such as one or more of the adenoviral regions E1, E2 and E4, therefore rendering the adenovirus to be incapable of replication. The deletion of the E1 region may comprise deletion of EIA, EIB 55K or EIB 21K, or any combination thereof. Replication deficient adenoviruses are propagated by providing the proteins encoded by the deleted region(s) in trans by the producer cell by utilizing helper plasmids or engineering the produce cell to express the required proteins. Adenovirus vectors may also have a deletion in the E3 region, which is dispensable for replication, and hence such a deletion does not have to be complemented. The adenovirus vector of the disclosure may comprise a functional deletion or a complete removal of the E1 region and at least part of the E3 region. The adenovirus vector of the disclosure may further comprise a functional deletion or a complete removal of the E4 region and/or the E2 region. Suitable producer cells that can be utilized are human retina cells immortalized by E1, e.g. 911 or PER.C6 cells (see, e.g., U.S. Pat. No. 5,994,128), E1-transformed amniocytes (See, e.g., EP 1230354), E 1-transformed A549 cells (see e.g. Int. Pat. Publ. No. WO1998/39411, U.S. Pat. No. 5,891,690).

Exemplary vectors that may be used are Ad26 comprising a functional E1 coding region that is sufficient for viral replication, a deletion in the E3 coding region and a deletion in the E4 coding region, provided that E4 open reading frame 6/7 is not deleted (see e.g. U.S. Pat. No. 9,750,801)

In some embodiments, the adenovirus vector is a human adenovirus (Ad) vector. In some embodiments, the Ad vector is derived from Ad5. In some embodiments, the Ad vector is derived from Ad11. In some embodiments, the Ad vector is derived from Ad26. In some embodiments, the Ad vector is derived from Ad34. In some embodiments, the Ad vector is derived from Ad35. In some embodiments, the Ad vector is derived from Ad48. In some embodiments, the Ad vector is derived from Ad49. In some embodiments, the Ad vector is derived from Ad50.

In some embodiments, the adenovirus vector is a great ape adenovirus (GAd) vector. In some embodiments, the GAd vector is derived from GAd20. In some embodiments, the GAd vector is derived from GAd19. In some embodiments, the GAd vector is derived from GAd21. In some embodiments, the GAd vector is derived from GAd25. In some embodiments, the GAd vector is derived from GAd26. In some embodiments, the GAd vector is derived from GAd27. In some embodiments, the GAd vector is derived from GAd28. In some embodiments, the GAd vector is derived from GAd29. In some embodiments, the GAd vector is derived from GAd30. In some embodiments, the GAd vector is derived from GAd31. In some embodiments, the GAd vector is derived from ChAd4. In some embodiments, the GAd vector is derived from ChAd5. In some embodiments, the GAd vector is derived from ChAd6. In some embodiments, the GAd vector is derived from ChAd7. In some embodiments, the GAd vector is derived from ChAd8. In some embodiments, the GAd vector is derived from ChAd9. In some embodiments, the GAd vector is derived from ChAd20. In some embodiments, the GAd vector is derived from ChAd22. In some embodiments, the GAd vector is derived from ChAd24. In some embodiments, the GAd vector is derived from ChAd26. In some embodiments, the GAd vector is derived from ChAd30. In some embodiments, the GAd vector is derived from ChAd31. In some embodiments, the GAd vector is derived from ChAd32. In some embodiments, the GAd vector is derived from ChAd33. In some embodiments, the GAd vector is derived from ChAd37. In some embodiments, the GAd vector is derived from ChAd38. In some embodiments, the GAd vector is derived from ChAd44. In some embodiments, the GAd vector is derived from ChAd55. In some embodiments, the GAd vector is derived from ChAd63. In some embodiments, the GAd vector is derived from ChAd68. In some embodiments, the GAd vector is derived from ChAd73. In some embodiments, the GAd vector is derived from ChAd82. In some embodiments, the GAd vector is derived from ChAd83.

The polypeptide or the heterologous polypeptide of the disclosure may be inserted into a site or region (insertion region) in the vector that does not affect virus viability of the resultant recombinant virus. The polypeptide or the heterologous polypeptide of the disclosure may be inserted into the deleted E1 region in parallel (transcribed 5′ to 3′) or anti-parallel (transcribed in a 3′ to 5′ direction relative to the vector backbone) orientation. In addition, appropriate transcriptional regulatory elements that are capable of directing expression of the polypeptide or the heterologous polypeptide of the disclosure in the mammalian host cells that the vector is being prepared for use may be operatively linked to the polypeptide or the heterologous polypeptide of the disclosure. “Operatively linked” sequences include both expression control sequences that are contiguous with the nucleic acid sequences that they regulate and regulatory sequences that act in trans, or at a distance to control the regulated nucleic acid sequence.

Recombinant adenoviral particles may be prepared and propagated according to any conventional technique in the field of the art (e.g., Int. Pat. Publ. No. WO1996/17070) using a complementation cell line or a helper virus, which supplies in trans the missing viral genes necessary for viral replication. The cell lines 293 (Graham et al., 1977, J. Gen. Virol. 36: 59-72), PER.C6 (see e.g. U.S. Pat. No. 5,994,128), E1 A549 and 911 are commonly used to complement E1 deletions. Other cell lines have been engineered to complement defective vectors (Yeh, et al., 1996, J. Virol. 70: 559-565; Kroughak and Graham, 1995, Human Gene Ther. 6: 1575-1586; Wang, et al., 1995, Gene Ther. 2: 775-783; Lusky, et al., 1998, J. Virol. 72: 2022-203; EP 919627 and Int. Pat. Publ. No. WO1997/04119). The adenoviral particles may be recovered from the culture supernatant but also from the cells after lysis and optionally further purified according to standard techniques (e.g., chromatography, ultracentrifugation, as described in Int. Pat. Publ. No. WO1996/27677, Int. Pat.

Publ. No. WO1998/00524, Int. Pat. Publ. No. WO1998/26048 and Int. Pat. Publ. No. WO2000/50573). The construction and methods for propagating adenoviral vectors are also described in for example, U.S. Pat. Nos. 5,559,099, 5,837,511, 5,846,782, 5,851,806, 5,994,106, 5,994,128, 5,965,541, 5,981,225, 6,040,174, 6,020,191, and 6,113,913.

The disclosure provides a recombinant adenovirus comprising the vector of the disclosure. The disclosure also provides a recombinant human adenovirus (rAd) comprising the vector of the disclosure. The disclosure also provides a recombinant human adenovirus derived from serotype 26 (rAd26) comprising the vector of the disclosure.

Provided herein is a viral vector comprising any of the polynucleotides of the disclosure, wherein the vector is derived from hAd26 (also referred to has Ad26).

In some embodiments, the Ad26 vector comprises a polynucleotide encoding an amino acid sequence of SEQ ID NO: 1 or having at least 90% sequence identity, or at least 95% sequence identity, or at least 99% sequence identity to SEQ ID NO: 1.

In some embodiments, the Ad26 vector comprises a polynucleotide encoding an amino acid sequence of SEQ ID NO: 3 or having at least 90% sequence identity, or at least 95% sequence identity, or at least 99% sequence identity to SEQ ID NO: 3.

In some embodiments, the Ad26 vector comprises a polynucleotide encoding an amino acid sequence of SEQ ID NO: 5 or having at least 90% sequence identity, or at least 95% sequence identity, or at least 99% sequence identity to SEQ ID NO: 5.

In some embodiments, the Ad26 vector comprises a polynucleotide encoding an amino acid sequence of SEQ ID NO: 7 or having at least 90% sequence identity, or at least 95% sequence identity, or at least 99% sequence identity to SEQ ID NO: 7.

In some embodiments, the Ad26 vector comprises a polynucleotide encoding an amino acid sequence of SEQ ID NO: 9 or having at least 90% sequence identity, or at least 95% sequence identity, or at least 99% sequence identity to SEQ ID NO: 9.

In some embodiments, the Ad26 vector comprises a polynucleotide encoding an amino acid sequence of SEQ ID NO: 11 or having at least 90% sequence identity, or at least 95% sequence identity, or at least 99% sequence identity to SEQ ID NO: 11.

In some embodiments, the Ad26 vector comprises a polynucleotide encoding an amino acid sequence of SEQ ID NO: 13 or having at least 90% sequence identity, or at least 95% sequence identity, or at least 99% sequence identity to SEQ ID NO: 13.

In some embodiments, the Ad26 vector comprises a polynucleotide encoding an amino acid sequence of SEQ ID NO: 15 or having at least 90% sequence identity, or at least 95% sequence identity, or at least 99% sequence identity to SEQ ID NO: 15.

In some embodiments, the Ad26 vector comprises a polynucleotide encoding an amino acid sequence of SEQ ID NO: 17 or having at least 90% sequence identity, or at least 95% sequence identity, or at least 99% sequence identity to SEQ ID NO: 17.

In some embodiments, the Ad26 vector comprises a polynucleotide encoding for a polypeptide of SEQ ID NO: 19 or having at least 90% sequence identity to SEQ ID NO: 19, or at least 95% sequence identity, or at least 99% sequence identity to SEQ ID NO: 19.

In some embodiments, the Ad26 vector comprises a polynucleotide encoding for a polypeptide of SEQ ID NO: 21 or having at least 90% sequence identity, or at least 95% sequence identity, or at least 99% sequence identity to SEQ ID NO: 21.

In some embodiments, the Ad26 vector comprises a polynucleotide encoding for a polypeptide of SEQ ID NO: 23 or having at least 90% sequence identity, or at least 95% sequence identity, or at least 99% sequence identity to SEQ ID NO: 23.

In some embodiments, the Ad26 vector comprises a polynucleotide encoding for a polypeptide encoding an amino acid sequence of SEQ ID NO: 25 or having at least 90% sequence identity, or at least 95% sequence identity, or at least 99% sequence identity to SEQ ID NO: 25.

In some embodiments, the Ad26 vector comprises a polynucleotide encoding for a polypeptide encoding an amino acid sequence of SEQ ID NO: 27 or having at least 90% sequence identity, or at least 95% sequence identity, or at least 99% sequence identity to SEQ ID NO: 27.

In some embodiments, the Ad26 vector comprises a polynucleotide encoding for a polypeptide encoding an amino acid sequence of SEQ ID NO: 29 or having at least 90% sequence identity, or at least 95% sequence identity, or at least 99% sequence identity to SEQ ID NO: 29.

In some embodiments, the Ad26 vector comprises a polynucleotide encoding for a polypeptide encoding an amino acid sequence of SEQ ID NO: 31 or having at least 90% sequence identity, or at least 95% sequence identity, or at least 99% sequence identity to SEQ ID NO: 31.

In some embodiments, the Ad26 vector comprises a polynucleotide encoding an amino acid sequence of SEQ ID NO: 33 or having at least 90% sequence identity, or at least 95% sequence identity, or at least 99% sequence identity to SEQ ID NO: 33.

In some embodiments, the Ad26 vector comprises a polynucleotide encoding an amino acid sequence of SEQ ID NO: 35 or having at least 90% sequence identity, or at least 95% sequence identity, or at least 99% sequence identity to SEQ ID NO: 35.

In some embodiments, the Ad26 vector comprises a polynucleotide encoding an amino acid sequence of SEQ ID NO: 37 or having at least 90% sequence identity, or at least 95% sequence identity, or at least 99% sequence identity to SEQ ID NO: 37.

In some embodiments, the Ad26 vector comprises a polynucleotide encoding an amino acid sequence of SEQ ID NO: 39 or having at least 90% sequence identity, or at least 95% sequence identity, or at least 99% sequence identity to SEQ ID NO: 39.

In some embodiments, the Ad26 vector comprises a polynucleotide encoding an amino acid sequence of SEQ ID NO: 41 or having at least 90% sequence identity, or at least 95% sequence identity, or at least 99% sequence identity to SEQ ID NO: 41.

In some embodiments, the Ad26 vector comprises a polynucleotide encoding an amino acid sequence of SEQ ID NO: 43 or having at least 90% sequence identity, or at least 95% sequence identity, or at least 99% sequence identity to SEQ ID NO: 43.

In some embodiments, the Ad26 vector comprises a polynucleotide encoding an amino acid sequence of SEQ ID NO: 45 or having at least 90% sequence identity, or at least 95% sequence identity, or at least 99% sequence identity to SEQ ID NO: 45. In some embodiments, the Ad26 vector comprises a polynucleotide encoding an amino acid sequence of SEQ ID NO: 47 or having at least 90% sequence identity, or at least 95% sequence identity, or at least 99% sequence identity to SEQ ID NO: 47.

In some embodiments, the Ad26 vector comprises a polynucleotide encoding an amino acid sequence of SEQ ID NO: 49 or having at least 90% sequence identity, or at least 95% sequence identity, or at least 99% sequence identity to SEQ ID NO: 49.

In some embodiments, the Ad26 vector comprises a polynucleotide encoding an amino acid sequence of SEQ ID NO: 51 or having at least 90% sequence identity, or at least 95% sequence identity, or at least 99% sequence identity to SEQ ID NO: 51.

In some embodiments, the Ad26 vector comprises a polynucleotide encoding an amino acid sequence of SEQ ID NO: 53 or having at least 90% sequence identity, or at least 95% sequence identity, or at least 99% sequence identity to SEQ ID NO: 53.

In some embodiments, the Ad26 vector comprises a polynucleotide encoding an amino acid sequence of SEQ ID NO: 55 or having at least 90% sequence identity, or at least 95% sequence identity, or at least 99% sequence identity to SEQ ID NO: 55.

In some embodiments, the Ad26 vector comprises a polynucleotide encoding an amino acid sequence of SEQ ID NO: 57 or having at least 90% sequence identity, or at least 95% sequence identity, or at least 99% sequence identity to SEQ ID NO: 57.

In some embodiments, the Ad26 vector comprises a polynucleotide encoding an amino acid sequence of SEQ ID NO: 59 or having at least 90% sequence identity, or at least 95% sequence identity, or at least 99% sequence identity to SEQ ID NO: 59.

In some embodiments, the Ad26 vector comprises a polynucleotide encoding an amino acid sequence of SEQ ID NO: 61 or having at least 90% sequence identity, or at least 95% sequence identity, or at least 99% sequence identity to SEQ ID NO: 61.

In some embodiments, the Ad26 vector comprises a polynucleotide encoding an amino acid sequence of SEQ ID NO: 63 or having at least 90% sequence identity, or at least 95% sequence identity, or at least 99% sequence identity to SEQ ID NO: 63.

In some embodiments, the Ad26 vector comprises a polynucleotide encoding an amino acid sequence of SEQ ID NO: 65 or having at least 90% sequence identity, or at least 95% sequence identity, or at least 99% sequence identity to SEQ ID NO: 65.

In some embodiments, the Ad26 vector comprises a polynucleotide encoding an amino acid sequence of SEQ ID NO: 67 or having at least 90% sequence identity, or at least 95% sequence identity, or at least 99% sequence identity to SEQ ID NO: 67.

In some embodiments, the Ad26 vector comprises a polynucleotide encoding an amino acid sequence of SEQ ID NO: 69 or having at least 90% sequence identity, or at least 95% sequence identity, or at least 99% sequence identity to SEQ ID NO: 69.

In some embodiments, the Ad26 vector comprises a polynucleotide encoding an amino acid sequence of SEQ ID NO: 71 or having at least 90% sequence identity, or at least 95% sequence identity, or at least 99% sequence identity to SEQ ID NO: 71.

In some embodiments, the Ad26 vector comprises a polynucleotide encoding an amino acid sequence of SEQ ID NO: 73 or having at least 90% sequence identity, or at least 95% sequence identity, or at least 99% sequence identity to SEQ ID NO: 73.

In some embodiments, the Ad26 vector comprises a polynucleotide encoding an amino acid sequence of SEQ ID NO: 75 or having at least 90% sequence identity, or at least 95% sequence identity, or at least 99% sequence identity to SEQ ID NO: 75.

In some embodiments, the Ad26 vector comprises a polynucleotide encoding an amino acid sequence of SEQ ID NO: 77 or having at least 90% sequence identity, or at least 95% sequence identity, or at least 99% sequence identity to SEQ ID NO: 77.

In some embodiments, the Ad26 vector comprises a polynucleotide encoding an amino acid sequence of SEQ ID NO: 79 or having at least 90% sequence identity, or at least 95% sequence identity, or at least 99% sequence identity to SEQ ID NO: 79.

In some embodiments, the Ad26 vector comprises a polynucleotide encoding an amino acid sequence of SEQ ID NO: 81 or having at least 90% sequence identity, or at least 95% sequence identity, or at least 99% sequence identity to SEQ ID NO: 81.

In some embodiments, the Ad26 vector comprises a polynucleotide encoding an amino acid sequence of SEQ ID NO: 83 or having at least 90% sequence identity, or at least 95% sequence identity, or at least 99% sequence identity to SEQ ID NO: 83.

In some embodiments, the Ad26 vector comprises a polynucleotide encoding an amino acid sequence of SEQ ID NO: 85 or having at least 90% sequence identity, or at least 95% sequence identity, or at least 99% sequence identity to SEQ ID NO: 85.

In some embodiments, the Ad26 vector comprises a polynucleotide encoding an amino acid sequence of SEQ ID NO: 87 or having at least 90% sequence identity, or at least 95% sequence identity, or at least 99% sequence identity to SEQ ID NO: 87.

In some embodiments, the Ad26 vector comprises a polynucleotide encoding an amino acid sequence of SEQ ID NO: 89 or having at least 90% sequence identity, or at least 95% sequence identity, or at least 99% sequence identity to SEQ ID NO: 89.

In some embodiments, the Ad26 vector comprises a polynucleotide encoding an amino acid sequence of SEQ ID NO: 91 or having at least 90% sequence identity, or at least 95% sequence identity, or at least 99% sequence identity to SEQ ID NO: 91.

In some embodiments, the Ad26 vector comprises a polynucleotide encoding an amino acid sequence of SEQ ID NO: 93 or having at least 90% sequence identity, or at least 95% sequence identity, or at least 99% sequence identity to SEQ ID NO: 93.

In some embodiments, the Ad26 vector comprises a polynucleotide encoding an amino acid sequence of SEQ ID NO: 95 or having at least 90% sequence identity, or at least 95% sequence identity, or at least 99% sequence identity to SEQ ID NO: 95.

In some embodiments, the Ad26 vector comprises a polynucleotide encoding an amino acid sequence of SEQ ID NO: 97 or having at least 90% sequence identity, or at least 95% sequence identity, or at least 99% sequence identity to SEQ ID NO: 97.

In some embodiments, the Ad26 vector comprises a polynucleotide encoding an amino acid sequence of SEQ ID NO: 99 or having at least 90% sequence identity, or at least 95% sequence identity, or at least 99% sequence identity to SEQ ID NO: 99.

In some embodiments, the Ad26 vector comprises a polynucleotide encoding an amino acid sequence of SEQ ID NO: 101 or having at least 90% sequence identity, or at least 95% sequence identity, or at least 99% sequence identity to SEQ ID NO: 101.

In some embodiments, the Ad26 vector comprises a polynucleotide encoding an amino acid sequence of SEQ ID NO: 103 or having at least 90% sequence identity, or at least 95% sequence identity, or at least 99% sequence identity to SEQ ID NO: 103.

In some embodiments, the Ad26 vector comprises a polynucleotide encoding an amino acid sequence of SEQ ID NO: 105 or having at least 90% sequence identity, or at least 95% sequence identity, or at least 99% sequence identity to SEQ ID NO: 105.

In some embodiments, the Ad26 vector comprises a polynucleotide encoding an amino acid sequence of SEQ ID NO: 107 or having at least 90% sequence identity, or at least 95% sequence identity, or at least 99% sequence identity to SEQ ID NO: 107.

In some embodiments, the Ad26 vector comprises a polynucleotide encoding an amino acid sequence of SEQ ID NO: 109 or having at least 90% sequence identity, or at least 95% sequence identity, or at least 99% sequence identity to SEQ ID NO: 109.

In some embodiments, the Ad26 vector comprises a polynucleotide encoding an amino acid sequence of SEQ ID NO: 111 or having at least 90% sequence identity, or at least 95% sequence identity, or at least 99% sequence identity to SEQ ID NO: 111.

In some embodiments, the Ad26 vector comprises a polynucleotide encoding an amino acid sequence of SEQ ID NO: 113 or having at least 90% sequence identity, or at least 95% sequence identity, or at least 99% sequence identity to SEQ ID NO: 113.

In some embodiments, the Ad26 vector comprises a polynucleotide encoding an amino acid sequence of SEQ ID NO: 115 or having at least 90% sequence identity, or at least 95% sequence identity, or at least 99% sequence identity to SEQ ID NO: 115.

In some embodiments, the Ad26 vector comprises a polynucleotide encoding an amino acid sequence of SEQ ID NO: 117 or having at least 90% sequence identity, or at least 95% sequence identity, or at least 99% sequence identity to SEQ ID NO: 117.

In some embodiments, the Ad26 vector comprises a polynucleotide encoding an amino acid sequence of SEQ ID NO: 119 or having at least 90% sequence identity, or at least 95% sequence identity, or at least 99% sequence identity to SEQ ID NO: 119.

In some embodiments, the Ad26 vector comprises a polynucleotide encoding an amino acid sequence of SEQ ID NO: 121 or having at least 90% sequence identity, or at least 95% sequence identity, or at least 99% sequence identity to SEQ ID NO: 121.

In some embodiments, the Ad26 vector comprises a polynucleotide encoding an amino acid sequence of SEQ ID NO: 123 or having at least 90% sequence identity, or at least 95% sequence identity, or at least 99% sequence identity to SEQ ID NO: 123.

In some embodiments, the Ad26 vector comprises a polynucleotide encoding an amino acid sequence of SEQ ID NO: 125 or having at least 90% sequence identity, or at least 95% sequence identity, or at least 99% sequence identity to SEQ ID NO: 125.

In some embodiments, the Ad26 vector comprises a polynucleotide encoding an amino acid sequence of SEQ ID NO: 127 or having at least 90% sequence identity, or at least 95% sequence identity, or at least 99% sequence identity to SEQ ID NO: 127.

In some embodiments, the Ad26 vector comprises a polynucleotide encoding an amino acid sequence of SEQ ID NO: 129 or having at least 90% sequence identity, or at least 95% sequence identity, or at least 99% sequence identity to SEQ ID NO: 129.

In some embodiments, the Ad26 vector comprises a polynucleotide encoding an amino acid sequence of SEQ ID NO: 131 or having at least 90% sequence identity, or at least 95% sequence identity, or at least 99% sequence identity to SEQ ID NO: 131.

In some embodiments, the Ad26 vector comprises a polynucleotide encoding an amino acid sequence of SEQ ID NO: 133 or having at least 90% sequence identity, or at least 95% sequence identity, or at least 99% sequence identity to SEQ ID NO: 133.

In some embodiments, the Ad26 vector comprises a polynucleotide encoding an amino acid sequence of SEQ ID NO: 135 or having at least 90% sequence identity, or at least 95% sequence identity, or at least 99% sequence identity to SEQ ID NO: 135.

In some embodiments, the Ad26 vector comprises a polynucleotide encoding an amino acid sequence of SEQ ID NO: 137 or having at least 90% sequence identity, or at least 95% sequence identity, or at least 99% sequence identity to SEQ ID NO: 137.

In some embodiments, the Ad26 vector comprises a polynucleotide encoding an amino acid sequence of SEQ ID NO: 139 or having at least 90% sequence identity, or at least 95% sequence identity, or at least 99% sequence identity to SEQ ID NO: 139.

In some embodiments, the Ad26 vector comprises a polynucleotide encoding an amino acid sequence of SEQ ID NO: 141 or having at least 90% sequence identity, or at least 95% sequence identity, or at least 99% sequence identity to SEQ ID NO: 141.

In some embodiments, the Ad26 vector comprises a polynucleotide encoding an amino acid sequence of SEQ ID NO: 143 or having at least 90% sequence identity, or at least 95% sequence identity, or at least 99% sequence identity to SEQ ID NO: 143.

In some embodiments, the Ad26 vector comprises a polynucleotide encoding an amino acid sequence of SEQ ID NO: 145 or having at least 90% sequence identity, or at least 95% sequence identity, or at least 99% sequence identity to SEQ ID NO: 145.

In some embodiments, the Ad26 vector comprises a polynucleotide encoding an amino acid sequence of SEQ ID NO: 147 or having at least 90% sequence identity, or at least 95% sequence identity, or at least 99% sequence identity to SEQ ID NO: 147.

In some embodiments, the Ad26 vector comprises a polynucleotide encoding an amino acid sequence of SEQ ID NO: 149 or having at least 90% sequence identity, or at least 95% sequence identity, or at least 99% sequence identity to SEQ ID NO: 149.

In some embodiments, the Ad26 vector comprises a polynucleotide encoding an amino acid sequence of SEQ ID NO: 151 or having at least 90% sequence identity, or at least 95% sequence identity, or at least 99% sequence identity to SEQ ID NO: 151.

In some embodiments, the Ad26 vector comprises a polynucleotide encoding an amino acid sequence of SEQ ID NO: 153 or having at least 90% sequence identity, or at least 95% sequence identity, or at least 99% sequence identity to SEQ ID NO: 153.

In some embodiments, the Ad26 vector comprises a polynucleotide encoding an amino acid sequence of SEQ ID NO: 155 or having at least 90% sequence identity, or at least 95% sequence identity, or at least 99% sequence identity to SEQ ID NO: 155.

In some embodiments, the Ad26 vector comprises a polynucleotide encoding an amino acid sequence of SEQ ID NO: 157 or having at least 90% sequence identity, or at least 95% sequence identity, or at least 99% sequence identity to SEQ ID NO: 157.

In some embodiments, the Ad26 vector comprises a polynucleotide encoding an amino acid sequence of SEQ ID NO: 159 or having at least 90% sequence identity, or at least 95% sequence identity, or at least 99% sequence identity to SEQ ID NO: 159.

In some embodiments, the Ad26 vector comprises a polynucleotide encoding an amino acid sequence of SEQ ID NO: 161 or having at least 90% sequence identity, or at least 95% sequence identity, or at least 99% sequence identity to SEQ ID NO: 161.

In some embodiments, the Ad26 vector comprises a polynucleotide encoding an amino acid sequence of SEQ ID NO: 163 or having at least 90% sequence identity, or at least 95% sequence identity, or at least 99% sequence identity to SEQ ID NO: 163.

In some embodiments, the Ad26 vector comprises a polynucleotide encoding an amino acid sequence of SEQ ID NO: 165 or having at least 90% sequence identity, or at least 95% sequence identity, or at least 99% sequence identity to SEQ ID NO: 165.

In some embodiments, the Ad26 vector comprises a polynucleotide encoding an amino acid sequence of SEQ ID NO: 167 or having at least 90% sequence identity, or at least 95% sequence identity, or at least 99% sequence identity to SEQ ID NO: 167.

In some embodiments, the Ad26 vector comprises a polynucleotide encoding an amino acid sequence of SEQ ID NO: 169 or having at least 90% sequence identity, or at least 95% sequence identity, or at least 99% sequence identity to SEQ ID NO: 169.

In some embodiments, the Ad26 vector comprises a polynucleotide encoding an amino acid sequence of SEQ ID NO: 171 or having at least 90% sequence identity, or at least 95% sequence identity, or at least 99% sequence identity to SEQ ID NO: 171.

In some embodiments, the Ad26 vector comprises a polynucleotide encoding an amino acid sequence of SEQ ID NO: 173 or having at least 90% sequence identity, or at least 95% sequence identity, or at least 99% sequence identity to SEQ ID NO: 173.

In some embodiments, the Ad26 vector comprises a polynucleotide encoding an amino acid sequence of SEQ ID NO: 175 or having at least 90% sequence identity, or at least 95% sequence identity, or at least 99% sequence identity to SEQ ID NO: 175.

In some embodiments, the Ad26 vector comprises a polynucleotide encoding an amino acid sequence of SEQ ID NO: 177 or having at least 90% sequence identity, or at least 95% sequence identity, or at least 99% sequence identity to SEQ ID NO: 177.

In some embodiments, the Ad26 vector comprises a polynucleotide encoding an amino acid sequence of SEQ ID NO: 179 or having at least 90% sequence identity, or at least 95% sequence identity, or at least 99% sequence identity to SEQ ID NO: 179.

In some embodiments, the Ad26 vector comprises a polynucleotide encoding an amino acid sequence of SEQ ID NO: 181 or having at least 90% sequence identity, or at least 95% sequence identity, or at least 99% sequence identity to SEQ ID NO: 181.

In some embodiments, the Ad26 vector comprises a polynucleotide encoding an amino acid sequence of SEQ ID NO: 183 or having at least 90% sequence identity, or at least 95% sequence identity, or at least 99% sequence identity to SEQ ID NO: 183.

In some embodiments, the Ad26 vector comprises a polynucleotide encoding an amino acid sequence of SEQ ID NO: 185 or having at least 90% sequence identity, or at least 95% sequence identity, or at least 99% sequence identity to SEQ ID NO: 185.

In some embodiments, the Ad26 vector comprises a polynucleotide encoding an amino acid sequence of SEQ ID NO: 187 or having at least 90% sequence identity, or at least 95% sequence identity, or at least 99% sequence identity to SEQ ID NO: 187.

In some embodiments, the Ad26 vector comprises a polynucleotide encoding an amino acid sequence of SEQ ID NO: 189 or having at least 90% sequence identity, or at least 95% sequence identity, or at least 99% sequence identity to SEQ ID NO: 189.

In some embodiments, the Ad26 vector comprises a polynucleotide encoding an amino acid sequence of SEQ ID NO: 191 or having at least 90% sequence identity, or at least 95% sequence identity, or at least 99% sequence identity to SEQ ID NO: 191.

In some embodiments, the Ad26 vector comprises a polynucleotide encoding an amino acid sequence of SEQ ID NO: 193 or having at least 90% sequence identity, or at least 95% sequence identity, or at least 99% sequence identity to SEQ ID NO: 193.

In some embodiments, the Ad26 vector comprises a polynucleotide encoding an amino acid sequence of SEQ ID NO: 195 or having at least 90% sequence identity, or at least 95% sequence identity, or at least 99% sequence identity to SEQ ID NO: 195.

In some embodiments, the Ad26 vector comprises a polynucleotide encoding an amino acid sequence of SEQ ID NO: 197 or having at least 90% sequence identity, or at least 95% sequence identity, or at least 99% sequence identity to SEQ ID NO: 197.

In some embodiments, the Ad26 vector comprises a polynucleotide encoding an amino acid sequence of SEQ ID NO: 199 or having at least 90% sequence identity, or at least 95% sequence identity, or at least 99% sequence identity to SEQ ID NO: 199.

In some embodiments, the Ad26 vector comprises a polynucleotide encoding an amino acid sequence of SEQ ID NO: 201 or having at least 90% sequence identity, or at least 95% sequence identity, or at least 99% sequence identity to SEQ ID NO: 201.

In some embodiments, the Ad26 vector comprises a polynucleotide encoding an amino acid sequence of SEQ ID NO: 203 or having at least 90% sequence identity, or at least 95% sequence identity, or at least 99% sequence identity to SEQ ID NO: 203.

In some embodiments, the Ad26 vector comprises a polynucleotide encoding an amino acid sequence of SEQ ID NO: 205 or having at least 90% sequence identity, or at least 95% sequence identity, or at least 99% sequence identity to SEQ ID NO: 205.

In some embodiments, the Ad26 vector comprises a polynucleotide encoding an amino acid sequence of SEQ ID NO: 207 or having at least 90% sequence identity, or at least 95% sequence identity, or at least 99% sequence identity to SEQ ID NO: 207.

In some embodiments, the Ad26 vector comprises a polynucleotide encoding an amino acid sequence of SEQ ID NO: 209 or having at least 90% sequence identity, or at least 95% sequence identity, or at least 99% sequence identity to SEQ ID NO: 209.

In some embodiments, the Ad26 vector comprises a polynucleotide encoding an amino acid sequence of SEQ ID NO: 211 or having at least 90% sequence identity, or at least 95% sequence identity, or at least 99% sequence identity to SEQ ID NO: 211.

In some embodiments, the Ad26 vector comprises a polynucleotide encoding an amino acid sequence of SEQ ID NO: 213 or having at least 90% sequence identity, or at least 95% sequence identity, or at least 99% sequence identity to SEQ ID NO: 213.

In some embodiments, the Ad26 vector comprises a polynucleotide encoding an amino acid sequence of SEQ ID NO: 215 or having at least 90% sequence identity, or at least 95% sequence identity, or at least 99% sequence identity to SEQ ID NO: 215.

In some embodiments, the Ad26 vector comprises a polynucleotide encoding an amino acid sequence of SEQ ID NO: 217 or having at least 90% sequence identity, or at least 95% sequence identity, or at least 99% sequence identity to SEQ ID NO: 217.

In some embodiments, the Ad26 vector comprises a polynucleotide encoding an amino acid sequence of SEQ ID NO: 219 or having at least 90% sequence identity, or at least 95% sequence identity, or at least 99% sequence identity to SEQ ID NO: 219.

In some embodiments, the Ad26 vector comprises a polynucleotide encoding an amino acid sequence of SEQ ID NO: 221 or having at least 90% sequence identity, or at least 95% sequence identity, or at least 99% sequence identity to SEQ ID NO: 221.

In some embodiments, the Ad26 vector comprises a polynucleotide encoding an amino acid sequence of SEQ ID NO: 223 or having at least 90% sequence identity, or at least 95% sequence identity, or at least 99% sequence identity to SEQ ID NO: 223.

In some embodiments, the Ad26 vector comprises a polynucleotide encoding an amino acid sequence of SEQ ID NO: 225 or having at least 90% sequence identity, or at least 95% sequence identity, or at least 99% sequence identity to SEQ ID NO: 225.

In some embodiments, the Ad26 vector comprises a polynucleotide encoding an amino acid sequence of SEQ ID NO: 227 or having at least 90% sequence identity, or at least 95% sequence identity, or at least 99% sequence identity to SEQ ID NO: 227.

In some embodiments, the Ad26 vector comprises a polynucleotide encoding an amino acid sequence of SEQ ID NO: 229 or having at least 90% sequence identity, or at least 95% sequence identity, or at least 99% sequence identity to SEQ ID NO: 229.

In some embodiments, the Ad26 vector comprises a polynucleotide encoding an amino acid sequence of SEQ ID NO: 231 or having at least 90% sequence identity, or at least 95% sequence identity, or at least 99% sequence identity to SEQ ID NO: 231.

In some embodiments, the Ad26 vector comprises a polynucleotide encoding an amino acid sequence of SEQ ID NO: 233 or having at least 90% sequence identity, or at least 95% sequence identity, or at least 99% sequence identity to SEQ ID NO: 233.

In some embodiments, the Ad26 vector comprises a polynucleotide encoding an amino acid sequence of SEQ ID NO: 235 or having at least 90% sequence identity, or at least 95% sequence identity, or at least 99% sequence identity to SEQ ID NO: 235.

In some embodiments, the Ad26 vector comprises a polynucleotide encoding an amino acid sequence of SEQ ID NO: 237 or having at least 90% sequence identity, or at least 95% sequence identity, or at least 99% sequence identity to SEQ ID NO: 237.

In some embodiments, the Ad26 vector comprises a polynucleotide encoding an amino acid sequence of SEQ ID NO: 239 or having at least 90% sequence identity, or at least 95% sequence identity, or at least 99% sequence identity to SEQ ID NO: 239.

In some embodiments, the Ad26 vector comprises a polynucleotide encoding an amino acid sequence of SEQ ID NO: 241 or having at least 90% sequence identity, or at least 95% sequence identity, or at least 99% sequence identity to SEQ ID NO: 241.

In some embodiments, the Ad26 vector comprises a polynucleotide encoding an amino acid sequence of SEQ ID NO: 243 or having at least 90% sequence identity, or at least 95% sequence identity, or at least 99% sequence identity to SEQ ID NO: 243.

In some embodiments, the Ad26 vector comprises a polynucleotide encoding an amino acid sequence of SEQ ID NO: 245 or having at least 90% sequence identity, or at least 95% sequence identity, or at least 99% sequence identity to SEQ ID NO: 245.

In some embodiments, the Ad26 vector comprises a polynucleotide encoding an amino acid sequence of SEQ ID NO: 247 or having at least 90% sequence identity, or at least 95% sequence identity, or at least 99% sequence identity to SEQ ID NO: 247.

In some embodiments, the Ad26 vector comprises a polynucleotide encoding an amino acid sequence of SEQ ID NO: 249 or having at least 90% sequence identity, or at least 95% sequence identity, or at least 99% sequence identity to SEQ ID NO: 249.

In some embodiments, the Ad26 vector comprises a polynucleotide encoding an amino acid sequence of SEQ ID NO: 251 or having at least 90% sequence identity, or at least 95% sequence identity, or at least 99% sequence identity to SEQ ID NO: 251.

In some embodiments, the Ad26 vector comprises a polynucleotide encoding an amino acid sequence of SEQ ID NO: 253 or having at least 90% sequence identity, or at least 95% sequence identity, or at least 99% sequence identity to SEQ ID NO: 253.

In some embodiments, the Ad26 vector comprises a polynucleotide encoding an amino acid sequence of SEQ ID NO: 255 or having at least 90% sequence identity, or at least 95% sequence identity, or at least 99% sequence identity to SEQ ID NO: 255.

In some embodiments, the Ad26 vector comprises a polynucleotide encoding an amino acid sequence of SEQ ID NO: 257 or having at least 90% sequence identity, or at least 95% sequence identity, or at least 99% sequence identity to SEQ ID NO: 257.

In some embodiments, the Ad26 vector comprises a polynucleotide encoding an amino acid sequence of SEQ ID NO: 259 or having at least 90% sequence identity, or at least 95% sequence identity, or at least 99% sequence identity to SEQ ID NO: 259.

In some embodiments, the Ad26 vector comprises a polynucleotide encoding an amino acid sequence of SEQ ID NO: 261 or having at least 90% sequence identity, or at least 95% sequence identity, or at least 99% sequence identity to SEQ ID NO: 261.

In some embodiments, the Ad26 vector comprises a polynucleotide encoding an amino acid sequence of SEQ ID NO: 263 or having at least 90% sequence identity, or at least 95% sequence identity, or at least 99% sequence identity to SEQ ID NO: 263.

In some embodiments, the Ad26 vector comprises a polynucleotide encoding an amino acid sequence of SEQ ID NO: 265 or having at least 90% sequence identity, or at least 95% sequence identity, or at least 99% sequence identity to SEQ ID NO: 265.

In some embodiments, the Ad26 vector comprises a polynucleotide encoding an amino acid sequence of SEQ ID NO: 267 or having at least 90% sequence identity, or at least 95% sequence identity, or at least 99% sequence identity to SEQ ID NO: 267.

In some embodiments, the Ad26 vector comprises a polynucleotide encoding an amino acid sequence of SEQ ID NO: 269 or having at least 90% sequence identity, or at least 95% sequence identity, or at least 99% sequence identity to SEQ ID NO: 269.

In some embodiments, the Ad26 vector comprises a polynucleotide encoding an amino acid sequence of SEQ ID NO: 271 or having at least 90% sequence identity, or at least 95% sequence identity, or at least 99% sequence identity to SEQ ID NO: 271.

In some embodiments, the Ad26 vector comprises a polynucleotide encoding an amino acid sequence of SEQ ID NO: 273 or having at least 90% sequence identity, or at least 95% sequence identity, or at least 99% sequence identity to SEQ ID NO: 273.

In some embodiments, the Ad26 vector comprises a polynucleotide encoding an amino acid sequence of SEQ ID NO: 275 or having at least 90% sequence identity, or at least 95% sequence identity, or at least 99% sequence identity to SEQ ID NO: 275.

In some embodiments, the Ad26 vector comprises a polynucleotide encoding an amino acid sequence of SEQ ID NO: 277 or having at least 90% sequence identity, or at least 95% sequence identity, or at least 99% sequence identity to SEQ ID NO: 277.

In some embodiments, the Ad26 vector comprises a polynucleotide encoding an amino acid sequence of SEQ ID NO: 279 or having at least 90% sequence identity, or at least 95% sequence identity, or at least 99% sequence identity to SEQ ID NO: 279.

In some embodiments, the Ad26 vector comprises a polynucleotide encoding an amino acid sequence of SEQ ID NO: 281 or having at least 90% sequence identity, or at least 95% sequence identity, or at least 99% sequence identity to SEQ ID NO: 281.

In some embodiments, the Ad26 vector comprises a polynucleotide encoding an amino acid sequence of SEQ ID NO: 283 or having at least 90% sequence identity, or at least 95% sequence identity, or at least 99% sequence identity to SEQ ID NO: 283.

In some embodiments, the Ad26 vector comprises a polynucleotide encoding an amino acid sequence of SEQ ID NO: 285 or having at least 90% sequence identity, or at least 95% sequence identity, or at least 99% sequence identity to SEQ ID NO: 285.

In some embodiments, the Ad26 vector comprises a polynucleotide encoding an amino acid sequence of SEQ ID NO: 287 or having at least 90% sequence identity, or at least 95% sequence identity, or at least 99% sequence identity to SEQ ID NO: 287.

In some embodiments, the Ad26 vector comprises a polynucleotide encoding an amino acid sequence of SEQ ID NO: 289 or having at least 90% sequence identity, or at least 95% sequence identity, or at least 99% sequence identity to SEQ ID NO: 289.

In some embodiments, the Ad26 vector comprises a polynucleotide encoding an amino acid sequence of SEQ ID NO: 291 or having at least 90% sequence identity, or at least 95% sequence identity, or at least 99% sequence identity to SEQ ID NO: 291.

In some embodiments, the Ad26 vector comprises a polynucleotide encoding an amino acid sequence of SEQ ID NO: 293 or having at least 90% sequence identity, or at least 95% sequence identity, or at least 99% sequence identity to SEQ ID NO: 293.

In some embodiments, the Ad26 vector comprises a polynucleotide encoding an amino acid sequence of SEQ ID NO: 295 or having at least 90% sequence identity, or at least 95% sequence identity, or at least 99% sequence identity to SEQ ID NO: 295.

In some embodiments, the Ad26 vector comprises a polynucleotide encoding an amino acid sequence of SEQ ID NO: 297 or having at least 90% sequence identity, or at least 95% sequence identity, or at least 99% sequence identity to SEQ ID NO: 297.

In some embodiments, the Ad26 vector comprises a polynucleotide encoding an amino acid sequence of SEQ ID NO: 299 or having at least 90% sequence identity, or at least 95% sequence identity, or at least 99% sequence identity to SEQ ID NO: 299.

In some embodiments, the Ad26 vector comprises a polynucleotide encoding an amino acid sequence of SEQ ID NO: 301 or having at least 90% sequence identity, or at least 95% sequence identity, or at least 99% sequence identity to SEQ ID NO: 301.

In some embodiments, the Ad26 vector comprises a polynucleotide encoding an amino acid sequence of SEQ ID NO: 303 or having at least 90% sequence identity, or at least 95% sequence identity, or at least 99% sequence identity to SEQ ID NO: 303.

In some embodiments, the Ad26 vector comprises a polynucleotide encoding an amino acid sequence of SEQ ID NO: 305 or having at least 90% sequence identity, or at least 95% sequence identity, or at least 99% sequence identity to SEQ ID NO: 305.

In some embodiments, the Ad26 vector comprises a polynucleotide encoding an amino acid sequence of SEQ ID NO: 307 or having at least 90% sequence identity, or at least 95% sequence identity, or at least 99% sequence identity to SEQ ID NO: 307.

In some embodiments, the Ad26 vector comprises a polynucleotide encoding an amino acid sequence of SEQ ID NO: 309 or having at least 90% sequence identity, or at least 95% sequence identity, or at least 99% sequence identity to SEQ ID NO: 309.

In some embodiments, the Ad26 vector comprises a polynucleotide encoding an amino acid sequence of SEQ ID NO: 311 or having at least 90% sequence identity, or at least 95% sequence identity, or at least 99% sequence identity to SEQ ID NO: 311.

In some embodiments, the Ad26 vector comprises a polynucleotide encoding an amino acid sequence of SEQ ID NO: 313 or having at least 90% sequence identity, or at least 95% sequence identity, or at least 99% sequence identity to SEQ ID NO: 313.

In some embodiments, the Ad26 vector comprises a polynucleotide encoding an amino acid sequence of SEQ ID NO: 315 or having at least 90% sequence identity, or at least 95% sequence identity, or at least 99% sequence identity to SEQ ID NO: 315.

In some embodiments, the Ad26 vector comprises a polynucleotide encoding an amino acid sequence of SEQ ID NO: 317 or having at least 90% sequence identity, or at least 95% sequence identity, or at least 99% sequence identity to SEQ ID NO: 317.

In some embodiments, the Ad26 vector comprises a polynucleotide encoding an amino acid sequence of SEQ ID NO: 319 or having at least 90% sequence identity, or at least 95% sequence identity, or at least 99% sequence identity to SEQ ID NO: 319.

In some embodiments, the Ad26 vector comprises a polynucleotide encoding an amino acid sequence of SEQ ID NO: 321 or having at least 90% sequence identity, or at least 95% sequence identity, or at least 99% sequence identity to SEQ ID NO: 321.

In some embodiments, the Ad26 vector comprises a polynucleotide encoding an amino acid sequence of SEQ ID NO: 323 or having at least 90% sequence identity, or at least 95% sequence identity, or at least 99% sequence identity to SEQ ID NO: 323.

In some embodiments, the Ad26 vector comprises a polynucleotide encoding an amino acid sequence of SEQ ID NO: 325 or having at least 90% sequence identity, or at least 95% sequence identity, or at least 99% sequence identity to SEQ ID NO: 325.

In some embodiments, the Ad26 vector comprises a polynucleotide encoding an amino acid sequence of SEQ ID NO: 327 or having at least 90% sequence identity, or at least 95% sequence identity, or at least 99% sequence identity to SEQ ID NO: 327.

In some embodiments, the Ad26 vector comprises a polynucleotide encoding an amino acid sequence of SEQ ID NO: 329 or having at least 90% sequence identity, or at least 95% sequence identity, or at least 99% sequence identity to SEQ ID NO: 329.

In some embodiments, the Ad26 vector comprises a polynucleotide encoding an amino acid sequence of SEQ ID NO: 331 or having at least 90% sequence identity, or at least 95% sequence identity, or at least 99% sequence identity to SEQ ID NO: 331.

In some embodiments, the Ad26 vector comprises a polynucleotide encoding an amino acid sequence of SEQ ID NO: 333 or having at least 90% sequence identity, or at least 95% sequence identity, or at least 99% sequence identity to SEQ ID NO: 333.

In some embodiments, the Ad26 vector comprises a polynucleotide encoding an amino acid sequence of SEQ ID NO: 335 or having at least 90% sequence identity, or at least 95% sequence identity, or at least 99% sequence identity to SEQ ID NO: 335.

In some embodiments, the Ad26 vector comprises a polynucleotide encoding an amino acid sequence of SEQ ID NO: 337 or having at least 90% sequence identity, or at least 95% sequence identity, or at least 99% sequence identity to SEQ ID NO: 337.

In some embodiments, the Ad26 vector comprises a polynucleotide encoding an amino acid sequence of SEQ ID NO: 339 or having at least 90% sequence identity, or at least 95% sequence identity, or at least 99% sequence identity to SEQ ID NO: 339.

In some embodiments, the Ad26 vector comprises a polynucleotide encoding an amino acid sequence of SEQ ID NO: 341 or having at least 90% sequence identity, or at least 95% sequence identity, or at least 99% sequence identity to SEQ ID NO: 341.

In some embodiments, the Ad26 vector comprises a polynucleotide encoding an amino acid sequence of SEQ ID NO: 343 or having at least 90% sequence identity, or at least 95% sequence identity, or at least 99% sequence identity to SEQ ID NO: 343.

In some embodiments, the Ad26 vector comprises a polynucleotide encoding an amino acid sequence of SEQ ID NO: 345 or having at least 90% sequence identity, or at least 95% sequence identity, or at least 99% sequence identity to SEQ ID NO: 345.

In some embodiments, the Ad26 vector comprises a polynucleotide encoding an amino acid sequence of SEQ ID NO: 347 or having at least 90% sequence identity, or at least 95% sequence identity, or at least 99% sequence identity to SEQ ID NO: 347.

In some embodiments, the Ad26 vector comprises a polynucleotide encoding an amino acid sequence of SEQ ID NO: 349 or having at least 90% sequence identity, or at least 95% sequence identity, or at least 99% sequence identity to SEQ ID NO: 349.

In some embodiments, the Ad26 vector comprises a polynucleotide encoding an amino acid sequence of SEQ ID NO: 351 or having at least 90% sequence identity, or at least 95% sequence identity, or at least 99% sequence identity to SEQ ID NO: 351.

In some embodiments, the Ad26 vector comprises a polynucleotide encoding an amino acid sequence of SEQ ID NO: 353 or having at least 90% sequence identity, or at least 95% sequence identity, or at least 99% sequence identity to SEQ ID NO: 353.

In some embodiments, the Ad26 vector comprises a polynucleotide encoding an amino acid sequence of SEQ ID NO: 355 or having at least 90% sequence identity, or at least 95% sequence identity, or at least 99% sequence identity to SEQ ID NO: 355.

In some embodiments, the Ad26 vector comprises a polynucleotide encoding an amino acid sequence of SEQ ID NO: 357 or having at least 90% sequence identity, or at least 95% sequence identity, or at least 99% sequence identity to SEQ ID NO: 357.

In some embodiments, the Ad26 vector comprises a polynucleotide encoding an amino acid sequence of SEQ ID NO: 359 or having at least 90% sequence identity, or at least 95% sequence identity, or at least 99% sequence identity to SEQ ID NO: 359.

In some embodiments, the Ad26 vector comprises a polynucleotide encoding an amino acid sequence of SEQ ID NO: 361 or having at least 90% sequence identity, or at least 95% sequence identity, or at least 99% sequence identity to SEQ ID NO: 361.

In some embodiments, the Ad26 vector comprises a polynucleotide encoding an amino acid sequence of SEQ ID NO: 363 or having at least 90% sequence identity, or at least 95% sequence identity, or at least 99% sequence identity to SEQ ID NO: 363.

In some embodiments, the Ad26 vector comprises a polynucleotide encoding an amino acid sequence of SEQ ID NO: 365 or having at least 90% sequence identity, or at least 95% sequence identity, or at least 99% sequence identity to SEQ ID NO: 365.

In some embodiments, the Ad26 vector comprises a polynucleotide encoding an amino acid sequence of SEQ ID NO: 367 or having at least 90% sequence identity, or at least 95% sequence identity, or at least 99% sequence identity to SEQ ID NO: 367.

In some embodiments, the Ad26 vector comprises a polynucleotide encoding an amino acid sequence of SEQ ID NO: 369 or having at least 90% sequence identity, or at least 95% sequence identity, or at least 99% sequence identity to SEQ ID NO: 369.

In some embodiments, the Ad26 vector comprises a polynucleotide encoding an amino acid sequence of SEQ ID NO: 371 or having at least 90% sequence identity, or at least 95% sequence identity, or at least 99% sequence identity to SEQ ID NO: 371.

In some embodiments, the Ad26 vector comprises a polynucleotide encoding an amino acid sequence of SEQ ID NO: 373 or having at least 90% sequence identity, or at least 95% sequence identity, or at least 99% sequence identity to SEQ ID NO: 373.

In some embodiments, the Ad26 vector comprises a polynucleotide encoding an amino acid sequence of SEQ ID NO: 375 or having at least 90% sequence identity, or at least 95% sequence identity, or at least 99% sequence identity to SEQ ID NO: 375.

In some embodiments, the Ad26 vector comprises a polynucleotide encoding an amino acid sequence of SEQ ID NO: 377 or having at least 90% sequence identity, or at least 95% sequence identity, or at least 99% sequence identity to SEQ ID NO: 377.

In some embodiments, the Ad26 vector comprises a polynucleotide encoding an amino acid sequence of SEQ ID NO: 379 or having at least 90% sequence identity, or at least 95% sequence identity, or at least 99% sequence identity to SEQ ID NO: 379.

In some embodiments, the Ad26 vector comprises a polynucleotide encoding an amino acid sequence of SEQ ID NO: 381 or having at least 90% sequence identity, or at least 95% sequence identity, or at least 99% sequence identity to SEQ ID NO: 381.

In some embodiments, the Ad26 vector comprises a polynucleotide encoding an amino acid sequence of SEQ ID NO: 383 or having at least 90% sequence identity, or at least 95% sequence identity, or at least 99% sequence identity to SEQ ID NO: 383.

In some embodiments, the Ad26 vector comprises a polynucleotide encoding an amino acid sequence of SEQ ID NO: 385 or having at least 90% sequence identity, or at least 95% sequence identity, or at least 99% sequence identity to SEQ ID NO: 385.

In some embodiments, the Ad26 vector comprises a polynucleotide encoding an amino acid sequence of SEQ ID NO: 387 or having at least 90% sequence identity, or at least 95% sequence identity, or at least 99% sequence identity to SEQ ID NO: 387.

In some embodiments, the Ad26 vector comprises a polynucleotide encoding an amino acid sequence of SEQ ID NO: 389 or having at least 90% sequence identity, or at least 95% sequence identity, or at least 99% sequence identity to SEQ ID NO: 389.

In some embodiments, the Ad26 vector comprises a polynucleotide encoding an amino acid sequence of SEQ ID NO: 391 or having at least 90% sequence identity, or at least 95% sequence identity, or at least 99% sequence identity to SEQ ID NO: 391.

In some embodiments, the Ad26 vector comprises a polynucleotide encoding an amino acid sequence of SEQ ID NO: 393 or having at least 90% sequence identity, or at least 95% sequence identity, or at least 99% sequence identity to SEQ ID NO: 393.

In some embodiments, the Ad26 vector comprises a polynucleotide encoding an amino acid sequence of SEQ ID NO: 395 or having at least 90% sequence identity, or at least 95% sequence identity, or at least 99% sequence identity to SEQ ID NO: 395.

In some embodiments, the Ad26 vector comprises a polynucleotide encoding an amino acid sequence of SEQ ID NO: 397 or having at least 90% sequence identity, or at least 95% sequence identity, or at least 99% sequence identity to SEQ ID NO: 397.

In some embodiments, the Ad26 vector comprises a polynucleotide encoding an amino acid sequence of SEQ ID NO: 399 or having at least 90% sequence identity, or at least 95% sequence identity, or at least 99% sequence identity to SEQ ID NO: 399.

In some embodiments, the Ad26 vector comprises a polynucleotide encoding an amino acid sequence of SEQ ID NO: 401 or having at least 90% sequence identity, or at least 95% sequence identity, or at least 99% sequence identity to SEQ ID NO: 401.

In some embodiments, the Ad26 vector comprises a polynucleotide encoding an amino acid sequence of SEQ ID NO: 403 or having at least 90% sequence identity, or at least 95% sequence identity, or at least 99% sequence identity to SEQ ID NO: 403.

In some embodiments, the Ad26 vector comprises a polynucleotide encoding an amino acid sequence of SEQ ID NO: 405 or having at least 90% sequence identity, or at least 95% sequence identity, or at least 99% sequence identity to SEQ ID NO: 405.

In some embodiments, the Ad26 vector comprises a polynucleotide encoding an amino acid sequence of SEQ ID NO: 407 or having at least 90% sequence identity, or at least 95% sequence identity, or at least 99% sequence identity to SEQ ID NO: 407.

In some embodiments, the Ad26 vector comprises a polynucleotide encoding an amino acid sequence of SEQ ID NO: 421 or having at least 90% sequence identity, or at least 95% sequence identity, or at least 99% sequence identity to SEQ ID NO: 421.

In some embodiments, the Ad26 vector comprises a polynucleotide encoding an amino acid sequence of two or more of the polypeptides selected from SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, and 421, and fragments thereof.

The disclosure also provides a recombinant great ape adenovirus (rGAd) comprising the vector of the disclosure. In some embodiments, the rGAd is derived from GAd20. In some embodiments, the rGAd is derived from GAd19. In some embodiments, the rGAd is derived from GAd21. In some embodiments, the rGAd is derived from GAd25. In some embodiments, the rGAd is derived from GAd26. In some embodiments, the rGAd is derived from GAd27. In some embodiments, the rGAd is derived from GAd28. In some embodiments, the rGAd is derived from GAd29. In some embodiments, the rGAd is derived from GAd30. In some embodiments, the rGAd is derived from GAd31. GAd19-21 and GAd25-31 are described in Int. Pat. Publ. No. WO2019/008111 and represent strains with high immunogenicity and no pre-existing immunity in the general human population. The polynucleotide sequence of GAd20 genome is disclosed in WO2019/008111.

Provided herein is a recombinant chimpanzee adenovirus derived from serotype 20 (rChAd20) comprising the vector of the disclosure. In some embodiments, the viral vector comprises any of the polynucleotides of the disclosure, wherein the vector is derived from GAd20.

In some embodiments, the GAd20 vector comprises a polynucleotide encoding an amino acid sequence of SEQ ID NO: 1 or having at least 90% sequence identity, or at least 95% sequence identity, or at least 99% sequence identity to SEQ ID NO: 1.

In some embodiments, the GAd20 vector comprises a polynucleotide encoding an amino acid sequence of SEQ ID NO: 3 or having at least 90% sequence identity, or at least 95% sequence identity, or at least 99% sequence identity to SEQ ID NO: 3.

In some embodiments, the GAd20 vector comprises a polynucleotide encoding an amino acid sequence of SEQ ID NO: 5 or having at least 90% sequence identity, or at least 95% sequence identity, or at least 99% sequence identity to SEQ ID NO: 5.

In some embodiments, the GAd20 vector comprises a polynucleotide encoding an amino acid sequence of SEQ ID NO: 7 or having at least 90% sequence identity, or at least 95% sequence identity, or at least 99% sequence identity to SEQ ID NO: 7.

In some embodiments, the GAd20 vector comprises a polynucleotide encoding an amino acid sequence of SEQ ID NO: 9 or having at least 90% sequence identity, or at least 95% sequence identity, or at least 99% sequence identity to SEQ ID NO: 9.

In some embodiments, the GAd20 vector comprises a polynucleotide encoding an amino acid sequence of SEQ ID NO: 11 or having at least 90% sequence identity, or at least 95% sequence identity, or at least 99% sequence identity to SEQ ID NO: 11.

In some embodiments, the GAd20 vector comprises a polynucleotide encoding an amino acid sequence of SEQ ID NO: 13 or having at least 90% sequence identity, or at least 95% sequence identity, or at least 99% sequence identity to SEQ ID NO: 13.

In some embodiments, the GAd20 vector comprises a polynucleotide encoding an amino acid sequence of SEQ ID NO: 15 or having at least 90% sequence identity, or at least 95% sequence identity, or at least 99% sequence identity to SEQ ID NO: 15.

In some embodiments, the GAd20 vector comprises a polynucleotide encoding an amino acid sequence of SEQ ID NO: 17 or having at least 90% sequence identity, or at least 95% sequence identity, or at least 99% sequence identity to SEQ ID NO: 17.

In some embodiments, the GAd20 vector comprises a polynucleotide encoding an amino acid sequence of SEQ ID NO: 19 or having at least 90% sequence identity to SEQ ID NO: 19, or at least 95% sequence identity, or at least 99% sequence identity to SEQ ID NO: 19.

In some embodiments, the GAd20 vector comprises a polynucleotide encoding an amino acid sequence of SEQ ID NO: 21 or having at least 90% sequence identity, or at least 95% sequence identity, or at least 99% sequence identity to SEQ ID NO: 21.

In some embodiments, the GAd20 vector comprises a polynucleotide encoding an amino acid sequence of SEQ ID NO: 23 or having at least 90% sequence identity, or at least 95% sequence identity, or at least 99% sequence identity to SEQ ID NO: 23.

In some embodiments, the GAd20 vector comprises a polynucleotide encoding an amino acid sequence of SEQ ID NO: 25 or having at least 90% sequence identity, or at least 95% sequence identity, or at least 99% sequence identity to SEQ ID NO: 25.

In some embodiments, the GAd20 vector comprises a polynucleotide encoding an amino acid sequence of SEQ ID NO: 27 or having at least 90% sequence identity, or at least 95% sequence identity, or at least 99% sequence identity to SEQ ID NO: 27.

In some embodiments, the GAd20 vector comprises a polynucleotide encoding an amino acid sequence of SEQ ID NO: 29 or having at least 90% sequence identity, or at least 95% sequence identity, or at least 99% sequence identity to SEQ ID NO: 29.

In some embodiments, the GAd20 vector comprises a polynucleotide encoding an amino acid sequence of SEQ ID NO: 31 or having at least 90% sequence identity, or at least 95% sequence identity, or at least 99% sequence identity to SEQ ID NO: 31.

In some embodiments, the GAd20 vector comprises a polynucleotide encoding an amino acid sequence of SEQ ID NO: 33 or having at least 90% sequence identity, or at least 95% sequence identity, or at least 99% sequence identity to SEQ ID NO: 33.

In some embodiments, the GAd20 vector comprises a polynucleotide encoding an amino acid sequence of SEQ ID NO: 35 or having at least 90% sequence identity, or at least 95% sequence identity, or at least 99% sequence identity to SEQ ID NO: 35.

In some embodiments, the GAd20 vector comprises a polynucleotide encoding an amino acid sequence of SEQ ID NO: 37 or having at least 90% sequence identity, or at least 95% sequence identity, or at least 99% sequence identity to SEQ ID NO: 37.

In some embodiments, the GAd20 vector comprises a polynucleotide encoding an amino acid sequence of SEQ ID NO: 39 or having at least 90% sequence identity, or at least 95% sequence identity, or at least 99% sequence identity to SEQ ID NO: 39.

In some embodiments, the GAd20 vector comprises a polynucleotide encoding an amino acid sequence of SEQ ID NO: 41 or having at least 90% sequence identity, or at least 95% sequence identity, or at least 99% sequence identity to SEQ ID NO: 41.

In some embodiments, the GAd20 vector comprises a polynucleotide encoding an amino acid sequence of SEQ ID NO: 43 or having at least 90% sequence identity, or at least 95% sequence identity, or at least 99% sequence identity to SEQ ID NO: 43.

In some embodiments, the GAd20 vector comprises a polynucleotide encoding an amino acid sequence of SEQ ID NO: 45 or having at least 90% sequence identity, or at least 95% sequence identity, or at least 99% sequence identity to SEQ ID NO: 45.

In some embodiments, the GAd20 vector comprises a polynucleotide encoding an amino acid sequence of SEQ ID NO: 47 or having at least 90% sequence identity, or at least 95% sequence identity, or at least 99% sequence identity to SEQ ID NO: 47.

In some embodiments, the GAd20 vector comprises a polynucleotide encoding an amino acid sequence of SEQ ID NO: 49 or having at least 90% sequence identity, or at least 95% sequence identity, or at least 99% sequence identity to SEQ ID NO: 49.

In some embodiments, the GAd20 vector comprises a polynucleotide encoding an amino acid sequence of SEQ ID NO: 51 or having at least 90% sequence identity, or at least 95% sequence identity, or at least 99% sequence identity to SEQ ID NO: 51.

In some embodiments, the GAd20 vector comprises a polynucleotide encoding an amino acid sequence of SEQ ID NO: 53 or having at least 90% sequence identity, or at least 95% sequence identity, or at least 99% sequence identity to SEQ ID NO: 53.

In some embodiments, the GAd20 vector comprises a polynucleotide encoding an amino acid sequence of SEQ ID NO: 55 or having at least 90% sequence identity, or at least 95% sequence identity, or at least 99% sequence identity to SEQ ID NO: 55.

In some embodiments, the GAd20 vector comprises a polynucleotide encoding an amino acid sequence of SEQ ID NO: 57 or having at least 90% sequence identity, or at least 95% sequence identity, or at least 99% sequence identity to SEQ ID NO: 57.

In some embodiments, the GAd20 vector comprises a polynucleotide encoding an amino acid sequence of SEQ ID NO: 59 or having at least 90% sequence identity, or at least 95% sequence identity, or at least 99% sequence identity to SEQ ID NO: 59.

In some embodiments, the GAd20 vector comprises a polynucleotide encoding an amino acid sequence of SEQ ID NO: 61 or having at least 90% sequence identity, or at least 95% sequence identity, or at least 99% sequence identity to SEQ ID NO: 61.

In some embodiments, the GAd20 vector comprises a polynucleotide encoding an amino acid sequence of SEQ ID NO: 63 or having at least 90% sequence identity, or at least 95% sequence identity, or at least 99% sequence identity to SEQ ID NO: 63.

In some embodiments, the GAd20 vector comprises a polynucleotide encoding an amino acid sequence of SEQ ID NO: 65 or having at least 90% sequence identity, or at least 95% sequence identity, or at least 99% sequence identity to SEQ ID NO: 65.

In some embodiments, the GAd20 vector comprises a polynucleotide encoding an amino acid sequence of SEQ ID NO: 67 or having at least 90% sequence identity, or at least 95% sequence identity, or at least 99% sequence identity to SEQ ID NO: 67.

In some embodiments, the GAd20 vector comprises a polynucleotide encoding an amino acid sequence of SEQ ID NO: 69 or having at least 90% sequence identity, or at least 95% sequence identity, or at least 99% sequence identity to SEQ ID NO: 69.

In some embodiments, the GAd20 vector comprises a polynucleotide encoding an amino acid sequence of SEQ ID NO: 71 or having at least 90% sequence identity, or at least 95% sequence identity, or at least 99% sequence identity to SEQ ID NO: 71.

In some embodiments, the GAd20 vector comprises a polynucleotide encoding an amino acid sequence of SEQ ID NO: 73 or having at least 90% sequence identity, or at least 95% sequence identity, or at least 99% sequence identity to SEQ ID NO: 73.

In some embodiments, the GAd20 vector comprises a polynucleotide encoding an amino acid sequence of SEQ ID NO: 75 or having at least 90% sequence identity, or at least 95% sequence identity, or at least 99% sequence identity to SEQ ID NO: 75.

In some embodiments, the GAd20 vector comprises a polynucleotide encoding an amino acid sequence of SEQ ID NO: 77 or having at least 90% sequence identity, or at least 95% sequence identity, or at least 99% sequence identity to SEQ ID NO: 77.

In some embodiments, the GAd20 vector comprises a polynucleotide encoding an amino acid sequence of SEQ ID NO: 79 or having at least 90% sequence identity, or at least 95% sequence identity, or at least 99% sequence identity to SEQ ID NO: 79.

In some embodiments, the GAd20 vector comprises a polynucleotide encoding an amino acid sequence of SEQ ID NO: 81 or having at least 90% sequence identity, or at least 95% sequence identity, or at least 99% sequence identity to SEQ ID NO: 81.

In some embodiments, the GAd20 vector comprises a polynucleotide encoding an amino acid sequence of SEQ ID NO: 83 or having at least 90% sequence identity, or at least 95% sequence identity, or at least 99% sequence identity to SEQ ID NO: 83.

In some embodiments, the GAd20 vector comprises a polynucleotide encoding an amino acid sequence of SEQ ID NO: 85 or having at least 90% sequence identity, or at least 95% sequence identity, or at least 99% sequence identity to SEQ ID NO: 85.

In some embodiments, the GAd20 vector comprises a polynucleotide encoding an amino acid sequence of SEQ ID NO: 87 or having at least 90% sequence identity, or at least 95% sequence identity, or at least 99% sequence identity to SEQ ID NO: 87.

In some embodiments, the GAd20 vector comprises a polynucleotide encoding an amino acid sequence of SEQ ID NO: 89 or having at least 90% sequence identity, or at least 95% sequence identity, or at least 99% sequence identity to SEQ ID NO: 89.

In some embodiments, the GAd20 vector comprises a polynucleotide encoding an amino acid sequence of SEQ ID NO: 91 or having at least 90% sequence identity, or at least 95% sequence identity, or at least 99% sequence identity to SEQ ID NO: 91.

In some embodiments, the GAd20 vector comprises a polynucleotide encoding an amino acid sequence of SEQ ID NO: 93 or having at least 90% sequence identity, or at least 95% sequence identity, or at least 99% sequence identity to SEQ ID NO: 93.

In some embodiments, the GAd20 vector comprises a polynucleotide encoding an amino acid sequence of SEQ ID NO: 95 or having at least 90% sequence identity, or at least 95% sequence identity, or at least 99% sequence identity to SEQ ID NO: 95.

In some embodiments, the GAd20 vector comprises a polynucleotide encoding an amino acid sequence of SEQ ID NO: 97 or having at least 90% sequence identity, or at least 95% sequence identity, or at least 99% sequence identity to SEQ ID NO: 97.

In some embodiments, the GAd20 vector comprises a polynucleotide encoding an amino acid sequence of SEQ ID NO: 99 or having at least 90% sequence identity, or at least 95% sequence identity, or at least 99% sequence identity to SEQ ID NO: 99.

In some embodiments, the GAd20 vector comprises a polynucleotide encoding an amino acid sequence of SEQ ID NO: 101 or having at least 90% sequence identity, or at least 95% sequence identity, or at least 99% sequence identity to SEQ ID NO: 101.

In some embodiments, the GAd20 vector comprises a polynucleotide encoding an amino acid sequence of SEQ ID NO: 103 or having at least 90% sequence identity, or at least 95% sequence identity, or at least 99% sequence identity to SEQ ID NO: 103.

In some embodiments, the GAd20 vector comprises a polynucleotide encoding an amino acid sequence of SEQ ID NO: 105 or having at least 90% sequence identity, or at least 95% sequence identity, or at least 99% sequence identity to SEQ ID NO: 105.

In some embodiments, the GAd20 vector comprises a polynucleotide encoding an amino acid sequence of SEQ ID NO: 107 or having at least 90% sequence identity, or at least 95% sequence identity, or at least 99% sequence identity to SEQ ID NO: 107.

In some embodiments, the GAd20 vector comprises a polynucleotide encoding an amino acid sequence of SEQ ID NO: 109 or having at least 90% sequence identity, or at least 95% sequence identity, or at least 99% sequence identity to SEQ ID NO: 109.

In some embodiments, the GAd20 vector comprises a polynucleotide encoding an amino acid sequence of SEQ ID NO: 111 or having at least 90% sequence identity, or at least 95% sequence identity, or at least 99% sequence identity to SEQ ID NO: 111.

In some embodiments, the GAd20 vector comprises a polynucleotide encoding an amino acid sequence of SEQ ID NO: 113 or having at least 90% sequence identity, or at least 95% sequence identity, or at least 99% sequence identity to SEQ ID NO: 113.

In some embodiments, the GAd20 vector comprises a polynucleotide encoding an amino acid sequence of SEQ ID NO: 115 or having at least 90% sequence identity, or at least 95% sequence identity, or at least 99% sequence identity to SEQ ID NO: 115.

In some embodiments, the GAd20 vector comprises a polynucleotide encoding an amino acid sequence of SEQ ID NO: 117 or having at least 90% sequence identity, or at least 95% sequence identity, or at least 99% sequence identity to SEQ ID NO: 117.

In some embodiments, the GAd20 vector comprises a polynucleotide encoding an amino acid sequence of SEQ ID NO: 119 or having at least 90% sequence identity, or at least 95% sequence identity, or at least 99% sequence identity to SEQ ID NO: 119.

In some embodiments, the GAd20 vector comprises a polynucleotide encoding an amino acid sequence of SEQ ID NO: 121 or having at least 90% sequence identity, or at least 95% sequence identity, or at least 99% sequence identity to SEQ ID NO: 121.

In some embodiments, the GAd20 vector comprises a polynucleotide encoding an amino acid sequence of SEQ ID NO: 123 or having at least 90% sequence identity, or at least 95% sequence identity, or at least 99% sequence identity to SEQ ID NO: 123.

In some embodiments, the GAd20 vector comprises a polynucleotide encoding an amino acid sequence of SEQ ID NO: 125 or having at least 90% sequence identity, or at least 95% sequence identity, or at least 99% sequence identity to SEQ ID NO: 125.

In some embodiments, the GAd20 vector comprises a polynucleotide encoding an amino acid sequence of SEQ ID NO: 127 or having at least 90% sequence identity, or at least 95% sequence identity, or at least 99% sequence identity to SEQ ID NO: 127.

In some embodiments, the GAd20 vector comprises a polynucleotide encoding an amino acid sequence of SEQ ID NO: 129 or having at least 90% sequence identity, or at least 95% sequence identity, or at least 99% sequence identity to SEQ ID NO: 129.

In some embodiments, the GAd20 vector comprises a polynucleotide encoding an amino acid sequence of SEQ ID NO: 131 or having at least 90% sequence identity, or at least 95% sequence identity, or at least 99% sequence identity to SEQ ID NO: 131.

In some embodiments, the GAd20 vector comprises a polynucleotide encoding an amino acid sequence of SEQ ID NO: 133 or having at least 90% sequence identity, or at least 95% sequence identity, or at least 99% sequence identity to SEQ ID NO: 133.

In some embodiments, the GAd20 vector comprises a polynucleotide encoding an amino acid sequence of SEQ ID NO: 135 or having at least 90% sequence identity, or at least 95% sequence identity, or at least 99% sequence identity to SEQ ID NO: 135.

In some embodiments, the GAd20 vector comprises a polynucleotide encoding an amino acid sequence of SEQ ID NO: 137 or having at least 90% sequence identity, or at least 95% sequence identity, or at least 99% sequence identity to SEQ ID NO: 137.

In some embodiments, the GAd20 vector comprises a polynucleotide encoding an amino acid sequence of SEQ ID NO: 139 or having at least 90% sequence identity, or at least 95% sequence identity, or at least 99% sequence identity to SEQ ID NO: 139.

In some embodiments, the GAd20 vector comprises a polynucleotide encoding an amino acid sequence of SEQ ID NO: 141 or having at least 90% sequence identity, or at least 95% sequence identity, or at least 99% sequence identity to SEQ ID NO: 141.

In some embodiments, the GAd20 vector comprises a polynucleotide encoding an amino acid sequence of SEQ ID NO: 143 or having at least 90% sequence identity, or at least 95% sequence identity, or at least 99% sequence identity to SEQ ID NO: 143.

In some embodiments, the GAd20 vector comprises a polynucleotide encoding an amino acid sequence of SEQ ID NO: 145 or having at least 90% sequence identity, or at least 95% sequence identity, or at least 99% sequence identity to SEQ ID NO: 145.

In some embodiments, the GAd20 vector comprises a polynucleotide encoding an amino acid sequence of SEQ ID NO: 147 or having at least 90% sequence identity, or at least 95% sequence identity, or at least 99% sequence identity to SEQ ID NO: 147.

In some embodiments, the GAd20 vector comprises a polynucleotide encoding an amino acid sequence of SEQ ID NO: 149 or having at least 90% sequence identity, or at least 95% sequence identity, or at least 99% sequence identity to SEQ ID NO: 149.

In some embodiments, the GAd20 vector comprises a polynucleotide encoding an amino acid sequence of SEQ ID NO: 151 or having at least 90% sequence identity, or at least 95% sequence identity, or at least 99% sequence identity to SEQ ID NO: 151.

In some embodiments, the GAd20 vector comprises a polynucleotide encoding an amino acid sequence of SEQ ID NO: 153 or having at least 90% sequence identity, or at least 95% sequence identity, or at least 99% sequence identity to SEQ ID NO: 153.

In some embodiments, the GAd20 vector comprises a polynucleotide encoding an amino acid sequence of SEQ ID NO: 155 or having at least 90% sequence identity, or at least 95% sequence identity, or at least 99% sequence identity to SEQ ID NO: 155.

In some embodiments, the GAd20 vector comprises a polynucleotide encoding an amino acid sequence of SEQ ID NO: 157 or having at least 90% sequence identity, or at least 95% sequence identity, or at least 99% sequence identity to SEQ ID NO: 157.

In some embodiments, the GAd20 vector comprises a polynucleotide encoding an amino acid sequence of SEQ ID NO: 159 or having at least 90% sequence identity, or at least 95% sequence identity, or at least 99% sequence identity to SEQ ID NO: 159.

In some embodiments, the GAd20 vector comprises a polynucleotide encoding an amino acid sequence of SEQ ID NO: 161 or having at least 90% sequence identity, or at least 95% sequence identity, or at least 99% sequence identity to SEQ ID NO: 161.

In some embodiments, the GAd20 vector comprises a polynucleotide encoding an amino acid sequence of SEQ ID NO: 163 or having at least 90% sequence identity, or at least 95% sequence identity, or at least 99% sequence identity to SEQ ID NO: 163.

In some embodiments, the GAd20 vector comprises a polynucleotide encoding an amino acid sequence of SEQ ID NO: 165 or having at least 90% sequence identity, or at least 95% sequence identity, or at least 99% sequence identity to SEQ ID NO: 165.

In some embodiments, the GAd20 vector comprises a polynucleotide encoding an amino acid sequence of SEQ ID NO: 167 or having at least 90% sequence identity, or at least 95% sequence identity, or at least 99% sequence identity to SEQ ID NO: 167.

In some embodiments, the GAd20 vector comprises a polynucleotide encoding an amino acid sequence of SEQ ID NO: 169 or having at least 90% sequence identity, or at least 95% sequence identity, or at least 99% sequence identity to SEQ ID NO: 169.

In some embodiments, the GAd20 vector comprises a polynucleotide encoding an amino acid sequence of SEQ ID NO: 171 or having at least 90% sequence identity, or at least 95% sequence identity, or at least 99% sequence identity to SEQ ID NO: 171.

In some embodiments, the GAd20 vector comprises a polynucleotide encoding an amino acid sequence of SEQ ID NO: 173 or having at least 90% sequence identity, or at least 95% sequence identity, or at least 99% sequence identity to SEQ ID NO: 173.

In some embodiments, the GAd20 vector comprises a polynucleotide encoding an amino acid sequence of SEQ ID NO: 175 or having at least 90% sequence identity, or at least 95% sequence identity, or at least 99% sequence identity to SEQ ID NO: 175.

In some embodiments, the GAd20 vector comprises a polynucleotide encoding an amino acid sequence of SEQ ID NO: 177 or having at least 90% sequence identity, or at least 95% sequence identity, or at least 99% sequence identity to SEQ ID NO: 177.

In some embodiments, the GAd20 vector comprises a polynucleotide encoding an amino acid sequence of SEQ ID NO: 179 or having at least 90% sequence identity, or at least 95% sequence identity, or at least 99% sequence identity to SEQ ID NO: 179.

In some embodiments, the GAd20 vector comprises a polynucleotide encoding an amino acid sequence of SEQ ID NO: 181 or having at least 90% sequence identity, or at least 95% sequence identity, or at least 99% sequence identity to SEQ ID NO: 181.

In some embodiments, the GAd20 vector comprises a polynucleotide encoding an amino acid sequence of SEQ ID NO: 183 or having at least 90% sequence identity, or at least 95% sequence identity, or at least 99% sequence identity to SEQ ID NO: 183.

In some embodiments, the GAd20 vector comprises a polynucleotide encoding an amino acid sequence of SEQ ID NO: 185 or having at least 90% sequence identity, or at least 95% sequence identity, or at least 99% sequence identity to SEQ ID NO: 185.

In some embodiments, the GAd20 vector comprises a polynucleotide encoding an amino acid sequence of SEQ ID NO: 187 or having at least 90% sequence identity, or at least 95% sequence identity, or at least 99% sequence identity to SEQ ID NO: 187.

In some embodiments, the GAd20 vector comprises a polynucleotide encoding an amino acid sequence of SEQ ID NO: 189 or having at least 90% sequence identity, or at least 95% sequence identity, or at least 99% sequence identity to SEQ ID NO: 189.

In some embodiments, the GAd20 vector comprises a polynucleotide encoding an amino acid sequence of SEQ ID NO: 191 or having at least 90% sequence identity, or at least 95% sequence identity, or at least 99% sequence identity to SEQ ID NO: 191.

In some embodiments, the GAd20 vector comprises a polynucleotide encoding an amino acid sequence of SEQ ID NO: 193 or having at least 90% sequence identity, or at least 95% sequence identity, or at least 99% sequence identity to SEQ ID NO: 193.

In some embodiments, the GAd20 vector comprises a polynucleotide encoding an amino acid sequence of SEQ ID NO: 195 or having at least 90% sequence identity, or at least 95% sequence identity, or at least 99% sequence identity to SEQ ID NO: 195.

In some embodiments, the GAd20 vector comprises a polynucleotide encoding an amino acid sequence of SEQ ID NO: 197 or having at least 90% sequence identity, or at least 95% sequence identity, or at least 99% sequence identity to SEQ ID NO: 197.

In some embodiments, the GAd20 vector comprises a polynucleotide encoding an amino acid sequence of SEQ ID NO: 199 or having at least 90% sequence identity, or at least 95% sequence identity, or at least 99% sequence identity to SEQ ID NO: 199.

In some embodiments, the GAd20 vector comprises a polynucleotide encoding an amino acid sequence of SEQ ID NO: 201 or having at least 90% sequence identity, or at least 95% sequence identity, or at least 99% sequence identity to SEQ ID NO: 201.

In some embodiments, the GAd20 vector comprises a polynucleotide encoding an amino acid sequence of SEQ ID NO: 203 or having at least 90% sequence identity, or at least 95% sequence identity, or at least 99% sequence identity to SEQ ID NO: 203.

In some embodiments, the GAd20 vector comprises a polynucleotide encoding an amino acid sequence of SEQ ID NO: 205 or having at least 90% sequence identity, or at least 95% sequence identity, or at least 99% sequence identity to SEQ ID NO: 205.

In some embodiments, the GAd20 vector comprises a polynucleotide encoding an amino acid sequence of SEQ ID NO: 207 or having at least 90% sequence identity, or at least 95% sequence identity, or at least 99% sequence identity to SEQ ID NO: 207.

In some embodiments, the GAd20 vector comprises a polynucleotide encoding an amino acid sequence of SEQ ID NO: 209 or having at least 90% sequence identity, or at least 95% sequence identity, or at least 99% sequence identity to SEQ ID NO: 209.

In some embodiments, the GAd20 vector comprises a polynucleotide encoding an amino acid sequence of SEQ ID NO: 211 or having at least 90% sequence identity, or at least 95% sequence identity, or at least 99% sequence identity to SEQ ID NO: 211.

In some embodiments, the GAd20 vector comprises a polynucleotide encoding an amino acid sequence of SEQ ID NO: 213 or having at least 90% sequence identity, or at least 95% sequence identity, or at least 99% sequence identity to SEQ ID NO: 213.

In some embodiments, the GAd20 vector comprises a polynucleotide encoding an amino acid sequence of SEQ ID NO: 215 or having at least 90% sequence identity, or at least 95% sequence identity, or at least 99% sequence identity to SEQ ID NO: 215.

In some embodiments, the GAd20 vector comprises a polynucleotide encoding an amino acid sequence of SEQ ID NO: 217 or having at least 90% sequence identity, or at least 95% sequence identity, or at least 99% sequence identity to SEQ ID NO: 217.

In some embodiments, the GAd20 vector comprises a polynucleotide encoding an amino acid sequence of SEQ ID NO: 219 or having at least 90% sequence identity, or at least 95% sequence identity, or at least 99% sequence identity to SEQ ID NO: 219.

In some embodiments, the GAd20 vector comprises a polynucleotide encoding an amino acid sequence of SEQ ID NO: 221 or having at least 90% sequence identity, or at least 95% sequence identity, or at least 99% sequence identity to SEQ ID NO: 221.

In some embodiments, the GAd20 vector comprises a polynucleotide encoding an amino acid sequence of SEQ ID NO: 223 or having at least 90% sequence identity, or at least 95% sequence identity, or at least 99% sequence identity to SEQ ID NO: 223.

In some embodiments, the GAd20 vector comprises a polynucleotide encoding an amino acid sequence of SEQ ID NO: 225 or having at least 90% sequence identity, or at least 95% sequence identity, or at least 99% sequence identity to SEQ ID NO: 225.

In some embodiments, the GAd20 vector comprises a polynucleotide encoding an amino acid sequence of SEQ ID NO: 227 or having at least 90% sequence identity, or at least 95% sequence identity, or at least 99% sequence identity to SEQ ID NO: 227.

In some embodiments, the GAd20 vector comprises a polynucleotide encoding an amino acid sequence of SEQ ID NO: 229 or having at least 90% sequence identity, or at least 95% sequence identity, or at least 99% sequence identity to SEQ ID NO: 229.

In some embodiments, the GAd20 vector comprises a polynucleotide encoding an amino acid sequence of SEQ ID NO: 231 or having at least 90% sequence identity, or at least 95% sequence identity, or at least 99% sequence identity to SEQ ID NO: 231.

In some embodiments, the GAd20 vector comprises a polynucleotide encoding an amino acid sequence of SEQ ID NO: 233 or having at least 90% sequence identity, or at least 95% sequence identity, or at least 99% sequence identity to SEQ ID NO: 233.

In some embodiments, the GAd20 vector comprises a polynucleotide encoding an amino acid sequence of SEQ ID NO: 235 or having at least 90% sequence identity, or at least 95% sequence identity, or at least 99% sequence identity to SEQ ID NO: 235.

In some embodiments, the GAd20 vector comprises a polynucleotide encoding an amino acid sequence of SEQ ID NO: 237 or having at least 90% sequence identity, or at least 95% sequence identity, or at least 99% sequence identity to SEQ ID NO: 237.

In some embodiments, the GAd20 vector comprises a polynucleotide encoding an amino acid sequence of SEQ ID NO: 239 or having at least 90% sequence identity, or at least 95% sequence identity, or at least 99% sequence identity to SEQ ID NO: 239.

In some embodiments, the GAd20 vector comprises a polynucleotide encoding an amino acid sequence of SEQ ID NO: 241 or having at least 90% sequence identity, or at least 95% sequence identity, or at least 99% sequence identity to SEQ ID NO: 241.

In some embodiments, the GAd20 vector comprises a polynucleotide encoding an amino acid sequence of SEQ ID NO: 243 or having at least 90% sequence identity, or at least 95% sequence identity, or at least 99% sequence identity to SEQ ID NO: 243.

In some embodiments, the GAd20 vector comprises a polynucleotide encoding an amino acid sequence of SEQ ID NO: 245 or having at least 90% sequence identity, or at least 95% sequence identity, or at least 99% sequence identity to SEQ ID NO: 245.

In some embodiments, the GAd20 vector comprises a polynucleotide encoding an amino acid sequence of SEQ ID NO: 247 or having at least 90% sequence identity, or at least 95% sequence identity, or at least 99% sequence identity to SEQ ID NO: 247.

In some embodiments, the GAd20 vector comprises a polynucleotide encoding an amino acid sequence of SEQ ID NO: 249 or having at least 90% sequence identity, or at least 95% sequence identity, or at least 99% sequence identity to SEQ ID NO: 249.

In some embodiments, the GAd20 vector comprises a polynucleotide encoding an amino acid sequence of SEQ ID NO: 251 or having at least 90% sequence identity, or at least 95% sequence identity, or at least 99% sequence identity to SEQ ID NO: 251.

In some embodiments, the GAd20 vector comprises a polynucleotide encoding an amino acid sequence of SEQ ID NO: 253 or having at least 90% sequence identity, or at least 95% sequence identity, or at least 99% sequence identity to SEQ ID NO: 253.

In some embodiments, the GAd20 vector comprises a polynucleotide encoding an amino acid sequence of SEQ ID NO: 255 or having at least 90% sequence identity, or at least 95% sequence identity, or at least 99% sequence identity to SEQ ID NO: 255.

In some embodiments, the GAd20 vector comprises a polynucleotide encoding an amino acid sequence of SEQ ID NO: 257 or having at least 90% sequence identity, or at least 95% sequence identity, or at least 99% sequence identity to SEQ ID NO: 257.

In some embodiments, the GAd20 vector comprises a polynucleotide encoding an amino acid sequence of SEQ ID NO: 259 or having at least 90% sequence identity, or at least 95% sequence identity, or at least 99% sequence identity to SEQ ID NO: 259.

In some embodiments, the GAd20 vector comprises a polynucleotide encoding an amino acid sequence of SEQ ID NO: 261 or having at least 90% sequence identity, or at least 95% sequence identity, or at least 99% sequence identity to SEQ ID NO: 261.

In some embodiments, the GAd20 vector comprises a polynucleotide encoding an amino acid sequence of SEQ ID NO: 263 or having at least 90% sequence identity, or at least 95% sequence identity, or at least 99% sequence identity to SEQ ID NO: 263.

In some embodiments, the GAd20 vector comprises a polynucleotide encoding an amino acid sequence of SEQ ID NO: 265 or having at least 90% sequence identity, or at least 95% sequence identity, or at least 99% sequence identity to SEQ ID NO: 265.

In some embodiments, the GAd20 vector comprises a polynucleotide encoding an amino acid sequence of SEQ ID NO: 267 or having at least 90% sequence identity, or at least 95% sequence identity, or at least 99% sequence identity to SEQ ID NO: 267.

In some embodiments, the GAd20 vector comprises a polynucleotide encoding an amino acid sequence of SEQ ID NO: 269 or having at least 90% sequence identity, or at least 95% sequence identity, or at least 99% sequence identity to SEQ ID NO: 269.

In some embodiments, the GAd20 vector comprises a polynucleotide encoding an amino acid sequence of SEQ ID NO: 271 or having at least 90% sequence identity, or at least 95% sequence identity, or at least 99% sequence identity to SEQ ID NO: 271.

In some embodiments, the GAd20 vector comprises a polynucleotide encoding an amino acid sequence of SEQ ID NO: 273 or having at least 90% sequence identity, or at least 95% sequence identity, or at least 99% sequence identity to SEQ ID NO: 273.

In some embodiments, the GAd20 vector comprises a polynucleotide encoding an amino acid sequence of SEQ ID NO: 275 or having at least 90% sequence identity, or at least 95% sequence identity, or at least 99% sequence identity to SEQ ID NO: 275.

In some embodiments, the GAd20 vector comprises a polynucleotide encoding an amino acid sequence of SEQ ID NO: 277 or having at least 90% sequence identity, or at least 95% sequence identity, or at least 99% sequence identity to SEQ ID NO: 277.

In some embodiments, the GAd20 vector comprises a polynucleotide encoding an amino acid sequence of SEQ ID NO: 279 or having at least 90% sequence identity, or at least 95% sequence identity, or at least 99% sequence identity to SEQ ID NO: 279.

In some embodiments, the GAd20 vector comprises a polynucleotide encoding an amino acid sequence of SEQ ID NO: 281 or having at least 90% sequence identity, or at least 95% sequence identity, or at least 99% sequence identity to SEQ ID NO: 281.

In some embodiments, the GAd20 vector comprises a polynucleotide encoding an amino acid sequence of SEQ ID NO: 283 or having at least 90% sequence identity, or at least 95% sequence identity, or at least 99% sequence identity to SEQ ID NO: 283.

In some embodiments, the GAd20 vector comprises a polynucleotide encoding an amino acid sequence of SEQ ID NO: 285 or having at least 90% sequence identity, or at least 95% sequence identity, or at least 99% sequence identity to SEQ ID NO: 285.

In some embodiments, the GAd20 vector comprises a polynucleotide encoding an amino acid sequence of SEQ ID NO: 287 or having at least 90% sequence identity, or at least 95% sequence identity, or at least 99% sequence identity to SEQ ID NO: 287.

In some embodiments, the GAd20 vector comprises a polynucleotide encoding an amino acid sequence of SEQ ID NO: 289 or having at least 90% sequence identity, or at least 95% sequence identity, or at least 99% sequence identity to SEQ ID NO: 289.

In some embodiments, the GAd20 vector comprises a polynucleotide encoding an amino acid sequence of SEQ ID NO: 291 or having at least 90% sequence identity, or at least 95% sequence identity, or at least 99% sequence identity to SEQ ID NO: 291.

In some embodiments, the GAd20 vector comprises a polynucleotide encoding an amino acid sequence of SEQ ID NO: 293 or having at least 90% sequence identity, or at least 95% sequence identity, or at least 99% sequence identity to SEQ ID NO: 293.

In some embodiments, the GAd20 vector comprises a polynucleotide encoding an amino acid sequence of SEQ ID NO: 295 or having at least 90% sequence identity, or at least 95% sequence identity, or at least 99% sequence identity to SEQ ID NO: 295.

In some embodiments, the GAd20 vector comprises a polynucleotide encoding an amino acid sequence of SEQ ID NO: 297 or having at least 90% sequence identity, or at least 95% sequence identity, or at least 99% sequence identity to SEQ ID NO: 297.

In some embodiments, the GAd20 vector comprises a polynucleotide encoding an amino acid sequence of SEQ ID NO: 299 or having at least 90% sequence identity, or at least 95% sequence identity, or at least 99% sequence identity to SEQ ID NO: 299.

In some embodiments, the GAd20 vector comprises a polynucleotide encoding an amino acid sequence of SEQ ID NO: 301 or having at least 90% sequence identity, or at least 95% sequence identity, or at least 99% sequence identity to SEQ ID NO: 301.

In some embodiments, the GAd20 vector comprises a polynucleotide encoding an amino acid sequence of SEQ ID NO: 303 or having at least 90% sequence identity, or at least 95% sequence identity, or at least 99% sequence identity to SEQ ID NO: 303.

In some embodiments, the GAd20 vector comprises a polynucleotide encoding an amino acid sequence of SEQ ID NO: 305 or having at least 90% sequence identity, or at least 95% sequence identity, or at least 99% sequence identity to SEQ ID NO: 305.

In some embodiments, the GAd20 vector comprises a polynucleotide encoding an amino acid sequence of SEQ ID NO: 307 or having at least 90% sequence identity, or at least 95% sequence identity, or at least 99% sequence identity to SEQ ID NO: 307.

In some embodiments, the GAd20 vector comprises a polynucleotide encoding an amino acid sequence of SEQ ID NO: 309 or having at least 90% sequence identity, or at least 95% sequence identity, or at least 99% sequence identity to SEQ ID NO: 309.

In some embodiments, the GAd20 vector comprises a polynucleotide encoding an amino acid sequence of SEQ ID NO: 311 or having at least 90% sequence identity, or at least 95% sequence identity, or at least 99% sequence identity to SEQ ID NO: 311.

In some embodiments, the GAd20 vector comprises a polynucleotide encoding an amino acid sequence of SEQ ID NO: 313 or having at least 90% sequence identity, or at least 95% sequence identity, or at least 99% sequence identity to SEQ ID NO: 313.

In some embodiments, the GAd20 vector comprises a polynucleotide encoding an amino acid sequence of SEQ ID NO: 315 or having at least 90% sequence identity, or at least 95% sequence identity, or at least 99% sequence identity to SEQ ID NO: 315.

In some embodiments, the GAd20 vector comprises a polynucleotide encoding an amino acid sequence of SEQ ID NO: 317 or having at least 90% sequence identity, or at least 95% sequence identity, or at least 99% sequence identity to SEQ ID NO: 317.

In some embodiments, the GAd20 vector comprises a polynucleotide encoding an amino acid sequence of SEQ ID NO: 319 or having at least 90% sequence identity, or at least 95% sequence identity, or at least 99% sequence identity to SEQ ID NO: 319.

In some embodiments, the GAd20 vector comprises a polynucleotide encoding an amino acid sequence of SEQ ID NO: 321 or having at least 90% sequence identity, or at least 95% sequence identity, or at least 99% sequence identity to SEQ ID NO: 321.

In some embodiments, the GAd20 vector comprises a polynucleotide encoding an amino acid sequence of SEQ ID NO: 323 or having at least 90% sequence identity, or at least 95% sequence identity, or at least 99% sequence identity to SEQ ID NO: 323.

In some embodiments, the GAd20 vector comprises a polynucleotide encoding an amino acid sequence of SEQ ID NO: 325 or having at least 90% sequence identity, or at least 95% sequence identity, or at least 99% sequence identity to SEQ ID NO: 325.

In some embodiments, the GAd20 vector comprises a polynucleotide encoding an amino acid sequence of SEQ ID NO: 327 or having at least 90% sequence identity, or at least 95% sequence identity, or at least 99% sequence identity to SEQ ID NO: 327.

In some embodiments, the GAd20 vector comprises a polynucleotide encoding an amino acid sequence of SEQ ID NO: 329 or having at least 90% sequence identity, or at least 95% sequence identity, or at least 99% sequence identity to SEQ ID NO: 329.

In some embodiments, the GAd20 vector comprises a polynucleotide encoding an amino acid sequence of SEQ ID NO: 331 or having at least 90% sequence identity, or at least 95% sequence identity, or at least 99% sequence identity to SEQ ID NO: 331.

In some embodiments, the GAd20 vector comprises a polynucleotide encoding an amino acid sequence of SEQ ID NO: 333 or having at least 90% sequence identity, or at least 95% sequence identity, or at least 99% sequence identity to SEQ ID NO: 333.

In some embodiments, the GAd20 vector comprises a polynucleotide encoding an amino acid sequence of SEQ ID NO: 335 or having at least 90% sequence identity, or at least 95% sequence identity, or at least 99% sequence identity to SEQ ID NO: 335.

In some embodiments, the GAd20 vector comprises a polynucleotide encoding an amino acid sequence of SEQ ID NO: 337 or having at least 90% sequence identity, or at least 95% sequence identity, or at least 99% sequence identity to SEQ ID NO: 337.

In some embodiments, the GAd20 vector comprises a polynucleotide encoding an amino acid sequence of SEQ ID NO: 339 or having at least 90% sequence identity, or at least 95% sequence identity, or at least 99% sequence identity to SEQ ID NO: 339.

In some embodiments, the GAd20 vector comprises a polynucleotide encoding an amino acid sequence of SEQ ID NO: 341 or having at least 90% sequence identity, or at least 95% sequence identity, or at least 99% sequence identity to SEQ ID NO: 341.

In some embodiments, the GAd20 vector comprises a polynucleotide encoding an amino acid sequence of SEQ ID NO: 343 or having at least 90% sequence identity, or at least 95% sequence identity, or at least 99% sequence identity to SEQ ID NO: 343.

In some embodiments, the GAd20 vector comprises a polynucleotide encoding an amino acid sequence of SEQ ID NO: 345 or having at least 90% sequence identity, or at least 95% sequence identity, or at least 99% sequence identity to SEQ ID NO: 345.

In some embodiments, the GAd20 vector comprises a polynucleotide encoding an amino acid sequence of SEQ ID NO: 347 or having at least 90% sequence identity, or at least 95% sequence identity, or at least 99% sequence identity to SEQ ID NO: 347. In some embodiments, the GAd20 vector comprises a polynucleotide encoding an amino acid sequence of SEQ ID NO: 349 or having at least 90% sequence identity, or at least 95% sequence identity, or at least 99% sequence identity to SEQ ID NO: 349.

In some embodiments, the GAd20 vector comprises a polynucleotide encoding an amino acid sequence of SEQ ID NO: 351 or having at least 90% sequence identity, or at least 95% sequence identity, or at least 99% sequence identity to SEQ ID NO: 351.

In some embodiments, the GAd20 vector comprises a polynucleotide encoding an amino acid sequence of SEQ ID NO: 353 or having at least 90% sequence identity, or at least 95% sequence identity, or at least 99% sequence identity to SEQ ID NO: 353.

In some embodiments, the GAd20 vector comprises a polynucleotide encoding an amino acid sequence of SEQ ID NO: 355 or having at least 90% sequence identity, or at least 95% sequence identity, or at least 99% sequence identity to SEQ ID NO: 355.

In some embodiments, the GAd20 vector comprises a polynucleotide encoding an amino acid sequence of SEQ ID NO: 357 or having at least 90% sequence identity, or at least 95% sequence identity, or at least 99% sequence identity to SEQ ID NO: 357. In some embodiments, the GAd20 vector comprises a polynucleotide encoding an amino acid sequence of SEQ ID NO: 359 or having at least 90% sequence identity, or at least 95% sequence identity, or at least 99% sequence identity to SEQ ID NO: 359.

In some embodiments, the GAd20 vector comprises a polynucleotide encoding an amino acid sequence of SEQ ID NO: 361 or having at least 90% sequence identity, or at least 95% sequence identity, or at least 99% sequence identity to SEQ ID NO: 361.

In some embodiments, the GAd20 vector comprises a polynucleotide encoding an amino acid sequence of SEQ ID NO: 363 or having at least 90% sequence identity, or at least 95% sequence identity, or at least 99% sequence identity to SEQ ID NO: 363.

In some embodiments, the GAd20 vector comprises a polynucleotide encoding an amino acid sequence of SEQ ID NO: 365 or having at least 90% sequence identity, or at least 95% sequence identity, or at least 99% sequence identity to SEQ ID NO: 365.

In some embodiments, the GAd20 vector comprises a polynucleotide encoding an amino acid sequence of SEQ ID NO: 367 or having at least 90% sequence identity, or at least 95% sequence identity, or at least 99% sequence identity to SEQ ID NO: 367.

In some embodiments, the GAd20 vector comprises a polynucleotide encoding an amino acid sequence of SEQ ID NO: 369 or having at least 90% sequence identity, or at least 95% sequence identity, or at least 99% sequence identity to SEQ ID NO: 369. In some embodiments, the GAd20 vector comprises a polynucleotide encoding an amino acid sequence of SEQ ID NO: 371 or having at least 90% sequence identity, or at least 95% sequence identity, or at least 99% sequence identity to SEQ ID NO: 371.

In some embodiments, the GAd20 vector comprises a polynucleotide encoding an amino acid sequence of SEQ ID NO: 373 or having at least 90% sequence identity, or at least 95% sequence identity, or at least 99% sequence identity to SEQ ID NO: 373.

In some embodiments, the GAd20 vector comprises a polynucleotide encoding an amino acid sequence of SEQ ID NO: 375 or having at least 90% sequence identity, or at least 95% sequence identity, or at least 99% sequence identity to SEQ ID NO: 375.

In some embodiments, the GAd20 vector comprises a polynucleotide encoding an amino acid sequence of SEQ ID NO: 377 or having at least 90% sequence identity, or at least 95% sequence identity, or at least 99% sequence identity to SEQ ID NO: 377.

In some embodiments, the GAd20 vector comprises a polynucleotide encoding an amino acid sequence of SEQ ID NO: 379 or having at least 90% sequence identity, or at least 95% sequence identity, or at least 99% sequence identity to SEQ ID NO: 379. In some embodiments, the GAd20 vector comprises a polynucleotide encoding an amino acid sequence of SEQ ID NO: 381 or having at least 90% sequence identity, or at least 95% sequence identity, or at least 99% sequence identity to SEQ ID NO: 381.

In some embodiments, the GAd20 vector comprises a polynucleotide encoding an amino acid sequence of SEQ ID NO: 383 or having at least 90% sequence identity, or at least 95% sequence identity, or at least 99% sequence identity to SEQ ID NO: 383.

In some embodiments, the GAd20 vector comprises a polynucleotide encoding an amino acid sequence of SEQ ID NO: 385 or having at least 90% sequence identity, or at least 95% sequence identity, or at least 99% sequence identity to SEQ ID NO: 385.

In some embodiments, the GAd20 vector comprises a polynucleotide encoding an amino acid sequence of SEQ ID NO: 387 or having at least 90% sequence identity, or at least 95% sequence identity, or at least 99% sequence identity to SEQ ID NO: 387.

In some embodiments, the GAd20 vector comprises a polynucleotide encoding an amino acid sequence of SEQ ID NO: 389 or having at least 90% sequence identity, or at least 95% sequence identity, or at least 99% sequence identity to SEQ ID NO: 389.

In some embodiments, the GAd20 vector comprises a polynucleotide encoding an amino acid sequence of SEQ ID NO: 391 or having at least 90% sequence identity, or at least 95% sequence identity, or at least 99% sequence identity to SEQ ID NO: 391.

In some embodiments, the GAd20 vector comprises a polynucleotide encoding an amino acid sequence of SEQ ID NO: 393 or having at least 90% sequence identity, or at least 95% sequence identity, or at least 99% sequence identity to SEQ ID NO: 393.

In some embodiments, the GAd20 vector comprises a polynucleotide encoding an amino acid sequence of SEQ ID NO: 395 or having at least 90% sequence identity, or at least 95% sequence identity, or at least 99% sequence identity to SEQ ID NO: 395.

In some embodiments, the GAd20 vector comprises a polynucleotide encoding an amino acid sequence of SEQ ID NO: 397 or having at least 90% sequence identity, or at least 95% sequence identity, or at least 99% sequence identity to SEQ ID NO: 397.

In some embodiments, the GAd20 vector comprises a polynucleotide encoding an amino acid sequence of SEQ ID NO: 399 or having at least 90% sequence identity, or at least 95% sequence identity, or at least 99% sequence identity to SEQ ID NO: 399.

In some embodiments, the GAd20 vector comprises a polynucleotide encoding an amino acid sequence of SEQ ID NO: 401 or having at least 90% sequence identity, or at least 95% sequence identity, or at least 99% sequence identity to SEQ ID NO: 401.

In some embodiments, the GAd20 vector comprises a polynucleotide encoding an amino acid sequence of SEQ ID NO: 403 or having at least 90% sequence identity, or at least 95% sequence identity, or at least 99% sequence identity to SEQ ID NO: 403.

In some embodiments, the GAd20 vector comprises a polynucleotide encoding an amino acid sequence of SEQ ID NO: 405 or having at least 90% sequence identity, or at least 95% sequence identity, or at least 99% sequence identity to SEQ ID NO: 405.

In some embodiments, the GAd20 vector comprises a polynucleotide encoding an amino acid sequence of SEQ ID NO: 405 or having at least 90% sequence identity, or at least 95% sequence identity, or at least 99% sequence identity to SEQ ID NO: 407.

In some embodiments, the GAd20 vector comprises a polynucleotide encoding an amino acid sequence of SEQ ID NO: 405 or having at least 90% sequence identity, or at least 95% sequence identity, or at least 99% sequence identity to SEQ ID NO: 421.

In some embodiments, the GAd20 vector comprises a polynucleotide encoding an amino acid sequence of two or more of the polypeptides selected from SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, and 421, and fragments thereof.

### Poxvirus Vectors

In some embodiments, the viral vector is derived from a poxvirus. In some embodiments, the recombinant virus comprising the vector is derived from a poxvirus.

Poxvirus (Poxviridae) vectors may be derived from smallpox virus (variola), vaccinia virus, cowpox virus or monkeypox virus. Exemplary vaccinia viruses are the Copenhagen vaccinia virus (W), New York Attenuated Vaccinia Virus (NYVAC), ALVAC, TROVAC and Modified Vaccinia Ankara (MVA).

MVA originates from the dermal vaccinia strain Ankara (Chorioallantois vaccinia Ankara (CVA) virus) that was maintained in the Vaccination Institute, Ankara, Turkey for many years and used as the basis for vaccination of humans. However, due to the often severe post-vaccinal complications associated with vaccinia viruses (VACV), there were several attempts to generate a more attenuated, safer smallpox vaccine.

MVA has been generated by 516 serial passages on chicken embryo fibroblasts of the CVA virus (see Meyer et al., J Gen. Virol., 72: 1031-1038 (1991) and U.S. Pat. No. 10,035,832). As a consequence of these long-term passages the resulting MVA virus deleted about 31 kilobases of its genomic sequence and, therefore, was described as highly host cell restricted to avian cells (Meyer, H. et al., Mapping of deletions in the genome of the highly attenuated vaccinia virus MVA and their influence on virulence, J. Gen. Virol. 72, 1031-1038, 1991; Meisinger-Henschel et al., Genomic sequence of chorioallantois vaccinia virus Ankara, the ancestor of modified vaccinia virus Ankara, J. Gen. Virol. 88, 3249-3259, 2007). Comparison of the MVA genome to its parent, CVA, revealed 6 major deletions of genomic DNA (deletion I, II, III, IV, V, and VI), totaling 31,000 basepairs. (Meyer et al., J. Gen. Virol. 72:1031-8 (1991)). It was shown in a variety of animal models that the resulting MVA was significantly avirulent (Mayr, A. & Danner, K. Vaccination against pox diseases under immunosuppressive conditions, Dev. Biol. Stand. 41: 225-34, 1978). Being that many passages were used to attenuate MVA, there are a number of different strains or isolates, depending on the passage number in CEF cells, such as MVA 476 MG/14/78, MVA-571, MVA-572, MVA-574, MVA-575 and MVA-BN. MVA 476 MG/14/78 is described for example in Int. Pat. Publ. No. WO2019/115816A1. MVA-572 strain was deposited at the European Collection of Animal Cell Cultures (“ECACC”), Health Protection Agency, Microbiology Services, Porton Down, Salisbury SP4 OJG, United Kingdom (“UK”), under the deposit number ECACC 94012707 on Jan. 27, 1994. MVA-575 strain was deposited at the ECACC under deposit number ECACC 00120707 on Dec. 7, 2000; MVA-Bavarian Nordic (“MVA-BN”) strain was deposited at the ECACC under deposit number V00080038 on Aug. 30, 2000. The genome sequences of MVA-BN and MVA-572 are available at GenBank (Accession numbers DQ983238 and DQ983237, respectively). The genome sequences of other MVA strains can be obtained using standard sequencing methods.

Vectors and viruses of the disclosure may be derived from any MVA strain or further derivatives of the MVA strain. A further exemplary MVA strain is deposit VR-1508, deposited at the American Type Culture collection (ATCC), Manassas, Va. 20108, USA.

“Derivatives” of MVA refer to viruses exhibiting essentially the same characteristics as the parent MVA but exhibiting differences in one or more parts of their genomes.

In some embodiments, the MVA vector is derived from MVA 476 MG/14/78 . In some embodiments, the MVA vector is derived from MVA-571. In some embodiments, the MVA vector is derived from MVA-572. In some embodiments, the MVA vector is derived from MVA-574. In some embodiments, the MVA vector is derived from MVA-575. In some embodiments, the MVA vector is derived from MVA-BN.

The polynucleotide or the heterologous polynucleotide of the disclosure may be inserted into a site or region (insertion region) in the MVA vector that does not affect virus viability of the resultant recombinant virus. Such regions can be readily identified by testing segments of virus DNA for regions that allow recombinant formation without seriously affecting virus viability of the recombinant virus. The thymidine kinase (TK) gene is an insertion region that may be used and is present in many viruses, such as in all examined poxvirus genomes. Additionally, MVA contains 6 natural deletion sites, each of which may be used as insertion sites (e.g. deletion I, II, III, IV, V, and VI; see e.g. U.S. Pat. Nos. 5,185,146 and6.440,442). One or more intergenic regions (IGR) of the MVA may also be used as an insertion site, such as IGRs IGR07/08, IGR 44/45, IGR 64/65, IGR 88/89, IGR 136/137, and IGR 148/149 (see e.g. U.S. Pat. Publ. No. 2018/0064803). Additional suitable insertion sites are described in Int. Pat. Publ. No. WO2005/048957.

Recombinant poxviral particles such as rMVA are prepared as described in the art (Piccini, et al., 1987, Methods of Enzymology 153: 545-563; U.S. Pat. Nos. 4,769,330; 4,772,848; 4,603,112; 5,100,587 and 5,179,993). In an exemplary method, the DNA sequence to be inserted into the virus can be placed into an E. coli plasmid construct into which DNA homologous to a section of DNA of the MVA has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of MVA DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA. Recombination between homologous MVA DNA in the plasmid and the viral genome, respectively, can generate an MVA modified by the presence of foreign DNA sequences. rMVA particles may be recovered from the culture supernatant or from the cultured cells after a lysis step (e.g., chemical lysis, freezing/thawing, osmotic shock, sonication and the like). Consecutive rounds of plaque purification can be used to remove contaminating wild type virus. Viral particles can then be purified using the techniques known in the art (e.g., chromatographic methods or ultracentrifugation on cesium chloride or sucrose gradients).

Provided herein is a viral vector comprising any of the polynucleotides of the disclosure, wherein the vector is derived from MVA. The disclosure also provides a recombinant modified vaccinia Ankara (rMVA) comprising the vector of the disclosure.

In some embodiments, the MVA vector comprises a polynucleotide encoding an amino acid sequence of SEQ ID NO: 1 or having at least 90% sequence identity, or at least 95% sequence identity, or at least 99% sequence identity to SEQ ID NO: 1.

In some embodiments, the MVA vector comprises a polynucleotide encoding an amino acid sequence of SEQ ID NO: 3 or having at least 90% sequence identity, or at least 95% sequence identity, or at least 99% sequence identity to SEQ ID NO: 3.

In some embodiments, the MVA vector comprises a polynucleotide encoding an amino acid sequence of SEQ ID NO: 5 or having at least 90% sequence identity, or at least 95% sequence identity, or at least 99% sequence identity to SEQ ID NO: 5.

In some embodiments, the MVA vector comprises a polynucleotide encoding an amino acid sequence of SEQ ID NO: 7 or having at least 90% sequence identity, or at least 95% sequence identity, or at least 99% sequence identity to SEQ ID NO: 7.

In some embodiments, the MVA vector comprises a polynucleotide encoding an amino acid sequence of SEQ ID NO: 9 or having at least 90% sequence identity, or at least 95% sequence identity, or at least 99% sequence identity to SEQ ID NO: 9.

In some embodiments, the MVA vector comprises a polynucleotide encoding an amino acid sequence of SEQ ID NO: 11 or having at least 90% sequence identity, or at least 95% sequence identity, or at least 99% sequence identity to SEQ ID NO: 11.

In some embodiments, the MVA vector comprises a polynucleotide encoding an amino acid sequence of SEQ ID NO: 13 or having at least 90% sequence identity, or at least 95% sequence identity, or at least 99% sequence identity to SEQ ID NO: 13.

In some embodiments, the MVA vector comprises a polynucleotide encoding an amino acid sequence of SEQ ID NO: 15 or having at least 90% sequence identity, or at least 95% sequence identity, or at least 99% sequence identity to SEQ ID NO: 15.

In some embodiments, the MVA vector comprises a polynucleotide encoding an amino acid sequence of SEQ ID NO: 17 or having at least 90% sequence identity, or at least 95% sequence identity, or at least 99% sequence identity to SEQ ID NO: 17.

In some embodiments, the MVA vector comprises a polynucleotide encoding an amino acid sequence of SEQ ID NO: 19 or having at least 90% sequence identity to SEQ ID NO: 19, or at least 95% sequence identity, or at least 99% sequence identity to SEQ ID NO: 19.

In some embodiments, the MVA vector comprises a polynucleotide encoding an amino acid sequence of SEQ ID NO: 21 or having at least 90% sequence identity, or at least 95% sequence identity, or at least 99% sequence identity to SEQ ID NO: 21.

In some embodiments, the MVA vector comprises a polynucleotide encoding an amino acid sequence of SEQ ID NO: 23 or having at least 90% sequence identity, or at least 95% sequence identity, or at least 99% sequence identity to SEQ ID NO: 23.

In some embodiments, the MVA vector comprises a polynucleotide encoding an amino acid sequence of SEQ ID NO: 25 or having at least 90% sequence identity, or at least 95% sequence identity, or at least 99% sequence identity to SEQ ID NO: 25.

In some embodiments, the MVA vector comprises a polynucleotide encoding an amino acid sequence of SEQ ID NO: 27 or having at least 90% sequence identity, or at least 95% sequence identity, or at least 99% sequence identity to SEQ ID NO: 27.

In some embodiments, the MVA vector comprises a polynucleotide encoding an amino acid sequence of SEQ ID NO: 29 or having at least 90% sequence identity, or at least 95% sequence identity, or at least 99% sequence identity to SEQ ID NO: 29.

In some embodiments, the MVA vector comprises a polynucleotide encoding an amino acid sequence of SEQ ID NO: 31 or having at least 90% sequence identity, or at least 95% sequence identity, or at least 99% sequence identity to SEQ ID NO: 31.

In some embodiments, the MVA vector comprises a polynucleotide encoding an amino acid sequence of SEQ ID NO: 33 or having at least 90% sequence identity, or at least 95% sequence identity, or at least 99% sequence identity to SEQ ID NO: 33.

In some embodiments, the MVA vector comprises a polynucleotide encoding an amino acid sequence of SEQ ID NO: 35 or having at least 90% sequence identity, or at least 95% sequence identity, or at least 99% sequence identity to SEQ ID NO: 35.

In some embodiments, the MVA vector comprises a polynucleotide encoding an amino acid sequence of SEQ ID NO: 37 or having at least 90% sequence identity, or at least 95% sequence identity, or at least 99% sequence identity to SEQ ID NO: 37.

In some embodiments, the MVA vector comprises a polynucleotide encoding an amino acid sequence of SEQ ID NO: 39 or having at least 90% sequence identity, or at least 95% sequence identity, or at least 99% sequence identity to SEQ ID NO: 39.

In some embodiments, the MVA vector comprises a polynucleotide encoding an amino acid sequence of SEQ ID NO: 41 or having at least 90% sequence identity, or at least 95% sequence identity, or at least 99% sequence identity to SEQ ID NO: 41.

In some embodiments, the MVA vector comprises a polynucleotide encoding an amino acid sequence of SEQ ID NO: 43 or having at least 90% sequence identity, or at least 95% sequence identity, or at least 99% sequence identity to SEQ ID NO: 43.

In some embodiments, the MVA vector comprises a polynucleotide encoding an amino acid sequence of SEQ ID NO: 45 or having at least 90% sequence identity, or at least 95% sequence identity, or at least 99% sequence identity to SEQ ID NO: 45.

In some embodiments, the MVA vector comprises a polynucleotide encoding an amino acid sequence of SEQ ID NO: 47 or having at least 90% sequence identity, or at least 95% sequence identity, or at least 99% sequence identity to SEQ ID NO: 47.

In some embodiments, the MVA vector comprises a polynucleotide encoding an amino acid sequence of SEQ ID NO: 49 or having at least 90% sequence identity, or at least 95% sequence identity, or at least 99% sequence identity to SEQ ID NO: 49.

In some embodiments, the MVA vector comprises a polynucleotide encoding an amino acid sequence of SEQ ID NO: 51 or having at least 90% sequence identity, or at least 95% sequence identity, or at least 99% sequence identity to SEQ ID NO: 51.

In some embodiments, the MVA vector comprises a polynucleotide encoding an amino acid sequence of SEQ ID NO: 53 or having at least 90% sequence identity, or at least 95% sequence identity, or at least 99% sequence identity to SEQ ID NO: 53.

In some embodiments, the MVA vector comprises a polynucleotide encoding an amino acid sequence of SEQ ID NO: 55 or having at least 90% sequence identity, or at least 95% sequence identity, or at least 99% sequence identity to SEQ ID NO: 55.

In some embodiments, the MVA vector comprises a polynucleotide encoding an amino acid sequence of SEQ ID NO: 57 or having at least 90% sequence identity, or at least 95% sequence identity, or at least 99% sequence identity to SEQ ID NO: 57.

In some embodiments, the MVA vector comprises a polynucleotide encoding an amino acid sequence of SEQ ID NO: 59 or having at least 90% sequence identity, or at least 95% sequence identity, or at least 99% sequence identity to SEQ ID NO: 59.

In some embodiments, the MVA vector comprises a polynucleotide encoding an amino acid sequence of SEQ ID NO: 61 or having at least 90% sequence identity, or at least 95% sequence identity, or at least 99% sequence identity to SEQ ID NO: 61.

In some embodiments, the MVA vector comprises a polynucleotide encoding an amino acid sequence of SEQ ID NO: 63 or having at least 90% sequence identity, or at least 95% sequence identity, or at least 99% sequence identity to SEQ ID NO: 63.

In some embodiments, the MVA vector comprises a polynucleotide encoding an amino acid sequence of SEQ ID NO: 65 or having at least 90% sequence identity, or at least 95% sequence identity, or at least 99% sequence identity to SEQ ID NO: 65.

In some embodiments, the MVA vector comprises a polynucleotide encoding an amino acid sequence of SEQ ID NO: 67 or having at least 90% sequence identity, or at least 95% sequence identity, or at least 99% sequence identity to SEQ ID NO: 67.

In some embodiments, the MVA vector comprises a polynucleotide encoding an amino acid sequence of SEQ ID NO: 69 or having at least 90% sequence identity, or at least 95% sequence identity, or at least 99% sequence identity to SEQ ID NO: 69.

In some embodiments, the MVA vector comprises a polynucleotide encoding an amino acid sequence of SEQ ID NO: 71 or having at least 90% sequence identity, or at least 95% sequence identity, or at least 99% sequence identity to SEQ ID NO: 71.

In some embodiments, the MVA vector comprises a polynucleotide encoding an amino acid sequence of SEQ ID NO: 73 or having at least 90% sequence identity, or at least 95% sequence identity, or at least 99% sequence identity to SEQ ID NO: 73.

In some embodiments, the MVA vector comprises a polynucleotide encoding an amino acid sequence of SEQ ID NO: 75 or having at least 90% sequence identity, or at least 95% sequence identity, or at least 99% sequence identity to SEQ ID NO: 75.

In some embodiments, the MVA vector comprises a polynucleotide encoding an amino acid sequence of SEQ ID NO: 77 or having at least 90% sequence identity, or at least 95% sequence identity, or at least 99% sequence identity to SEQ ID NO: 77.

In some embodiments, the MVA vector comprises a polynucleotide encoding an amino acid sequence of SEQ ID NO: 79 or having at least 90% sequence identity, or at least 95% sequence identity, or at least 99% sequence identity to SEQ ID NO: 79.

In some embodiments, the MVA vector comprises a polynucleotide encoding an amino acid sequence of SEQ ID NO: 81 or having at least 90% sequence identity, or at least 95% sequence identity, or at least 99% sequence identity to SEQ ID NO: 81.

In some embodiments, the MVA vector comprises a polynucleotide encoding an amino acid sequence of SEQ ID NO: 83 or having at least 90% sequence identity, or at least 95% sequence identity, or at least 99% sequence identity to SEQ ID NO: 83.

In some embodiments, the MVA vector comprises a polynucleotide encoding an amino acid sequence of SEQ ID NO: 85 or having at least 90% sequence identity, or at least 95% sequence identity, or at least 99% sequence identity to SEQ ID NO: 85.

In some embodiments, the MVA vector comprises a polynucleotide encoding an amino acid sequence of SEQ ID NO: 87 or having at least 90% sequence identity, or at least 95% sequence identity, or at least 99% sequence identity to SEQ ID NO: 87.

In some embodiments, the MVA vector comprises a polynucleotide encoding an amino acid sequence of SEQ ID NO: 89 or having at least 90% sequence identity, or at least 95% sequence identity, or at least 99% sequence identity to SEQ ID NO: 89.

In some embodiments, the MVA vector comprises a polynucleotide encoding an amino acid sequence of SEQ ID NO: 91 or having at least 90% sequence identity, or at least 95% sequence identity, or at least 99% sequence identity to SEQ ID NO: 91.

In some embodiments, the MVA vector comprises a polynucleotide encoding an amino acid sequence of SEQ ID NO: 93 or having at least 90% sequence identity, or at least 95% sequence identity, or at least 99% sequence identity to SEQ ID NO: 93.

In some embodiments, the MVA vector comprises a polynucleotide encoding an amino acid sequence of SEQ ID NO: 95 or having at least 90% sequence identity, or at least 95% sequence identity, or at least 99% sequence identity to SEQ ID NO: 95.

In some embodiments, the MVA vector comprises a polynucleotide encoding an amino acid sequence of SEQ ID NO: 97 or having at least 90% sequence identity, or at least 95% sequence identity, or at least 99% sequence identity to SEQ ID NO: 97.

In some embodiments, the MVA vector comprises a polynucleotide encoding an amino acid sequence of SEQ ID NO: 99 or having at least 90% sequence identity, or at least 95% sequence identity, or at least 99% sequence identity to SEQ ID NO: 99.

In some embodiments, the MVA vector comprises a polynucleotide encoding an amino acid sequence of SEQ ID NO: 101 or having at least 90% sequence identity, or at least 95% sequence identity, or at least 99% sequence identity to SEQ ID NO: 101.

In some embodiments, the MVA vector comprises a polynucleotide encoding an amino acid sequence of SEQ ID NO: 103 or having at least 90% sequence identity, or at least 95% sequence identity, or at least 99% sequence identity to SEQ ID NO: 103.

In some embodiments, the MVA vector comprises a polynucleotide encoding an amino acid sequence of SEQ ID NO: 105 or having at least 90% sequence identity, or at least 95% sequence identity, or at least 99% sequence identity to SEQ ID NO: 105.

In some embodiments, the MVA vector comprises a polynucleotide encoding an amino acid sequence of SEQ ID NO: 107 or having at least 90% sequence identity, or at least 95% sequence identity, or at least 99% sequence identity to SEQ ID NO: 107.

In some embodiments, the MVA vector comprises a polynucleotide encoding an amino acid sequence of SEQ ID NO: 109 or having at least 90% sequence identity, or at least 95% sequence identity, or at least 99% sequence identity to SEQ ID NO: 109.

In some embodiments, the MVA vector comprises a polynucleotide encoding an amino acid sequence of SEQ ID NO: 111 or having at least 90% sequence identity, or at least 95% sequence identity, or at least 99% sequence identity to SEQ ID NO: 111.

In some embodiments, the MVA vector comprises a polynucleotide encoding an amino acid sequence of SEQ ID NO: 113 or having at least 90% sequence identity, or at least 95% sequence identity, or at least 99% sequence identity to SEQ ID NO: 113.

In some embodiments, the MVA vector comprises a polynucleotide encoding an amino acid sequence of SEQ ID NO: 115 or having at least 90% sequence identity, or at least 95% sequence identity, or at least 99% sequence identity to SEQ ID NO: 115.

In some embodiments, the MVA vector comprises a polynucleotide encoding an amino acid sequence of SEQ ID NO: 117 or having at least 90% sequence identity, or at least 95% sequence identity, or at least 99% sequence identity to SEQ ID NO: 117.

In some embodiments, the MVA vector comprises a polynucleotide encoding an amino acid sequence of SEQ ID NO: 119 or having at least 90% sequence identity, or at least 95% sequence identity, or at least 99% sequence identity to SEQ ID NO: 119.

In some embodiments, the MVA vector comprises a polynucleotide encoding an amino acid sequence of SEQ ID NO: 121 or having at least 90% sequence identity, or at least 95% sequence identity, or at least 99% sequence identity to SEQ ID NO: 121.

In some embodiments, the MVA vector comprises a polynucleotide encoding an amino acid sequence of SEQ ID NO: 123 or having at least 90% sequence identity, or at least 95% sequence identity, or at least 99% sequence identity to SEQ ID NO: 123.

In some embodiments, the MVA vector comprises a polynucleotide encoding an amino acid sequence of SEQ ID NO: 125 or having at least 90% sequence identity, or at least 95% sequence identity, or at least 99% sequence identity to SEQ ID NO: 125.

In some embodiments, the MVA vector comprises a polynucleotide encoding an amino acid sequence of SEQ ID NO: 127 or having at least 90% sequence identity, or at least 95% sequence identity, or at least 99% sequence identity to SEQ ID NO: 127.

In some embodiments, the MVA vector comprises a polynucleotide encoding an amino acid sequence of SEQ ID NO: 129 or having at least 90% sequence identity, or at least 95% sequence identity, or at least 99% sequence identity to SEQ ID NO: 129.

In some embodiments, the MVA vector comprises a polynucleotide encoding an amino acid sequence of SEQ ID NO: 131 or having at least 90% sequence identity, or at least 95% sequence identity, or at least 99% sequence identity to SEQ ID NO: 131.

In some embodiments, the MVA vector comprises a polynucleotide encoding an amino acid sequence of SEQ ID NO: 133 or having at least 90% sequence identity, or at least 95% sequence identity, or at least 99% sequence identity to SEQ ID NO: 133.

In some embodiments, the MVA vector comprises a polynucleotide encoding an amino acid sequence of SEQ ID NO: 135 or having at least 90% sequence identity, or at least 95% sequence identity, or at least 99% sequence identity to SEQ ID NO: 135.

In some embodiments, the MVA vector comprises a polynucleotide encoding an amino acid sequence of SEQ ID NO: 137 or having at least 90% sequence identity, or at least 95% sequence identity, or at least 99% sequence identity to SEQ ID NO: 137.

In some embodiments, the MVA vector comprises a polynucleotide encoding an amino acid sequence of SEQ ID NO: 139 or having at least 90% sequence identity, or at least 95% sequence identity, or at least 99% sequence identity to SEQ ID NO: 139.

In some embodiments, the MVA vector comprises a polynucleotide encoding an amino acid sequence of SEQ ID NO: 141 or having at least 90% sequence identity, or at least 95% sequence identity, or at least 99% sequence identity to SEQ ID NO: 141.

In some embodiments, the MVA vector comprises a polynucleotide encoding an amino acid sequence of SEQ ID NO: 143 or having at least 90% sequence identity, or at least 95% sequence identity, or at least 99% sequence identity to SEQ ID NO: 143.

In some embodiments, the MVA vector comprises a polynucleotide encoding an amino acid sequence of SEQ ID NO: 145 or having at least 90% sequence identity, or at least 95% sequence identity, or at least 99% sequence identity to SEQ ID NO: 145.

In some embodiments, the MVA vector comprises a polynucleotide encoding an amino acid sequence of SEQ ID NO: 147 or having at least 90% sequence identity, or at least 95% sequence identity, or at least 99% sequence identity to SEQ ID NO: 147.

In some embodiments, the MVA vector comprises a polynucleotide encoding an amino acid sequence of SEQ ID NO: 149 or having at least 90% sequence identity, or at least 95% sequence identity, or at least 99% sequence identity to SEQ ID NO: 149.

In some embodiments, the MVA vector comprises a polynucleotide encoding an amino acid sequence of SEQ ID NO: 151 or having at least 90% sequence identity, or at least 95% sequence identity, or at least 99% sequence identity to SEQ ID NO: 151.

In some embodiments, the MVA vector comprises a polynucleotide encoding an amino acid sequence of SEQ ID NO: 153 or having at least 90% sequence identity, or at least 95% sequence identity, or at least 99% sequence identity to SEQ ID NO: 153.

In some embodiments, the MVA vector comprises a polynucleotide encoding an amino acid sequence of SEQ ID NO: 155 or having at least 90% sequence identity, or at least 95% sequence identity, or at least 99% sequence identity to SEQ ID NO: 155.

In some embodiments, the MVA vector comprises a polynucleotide encoding an amino acid sequence of SEQ ID NO: 157 or having at least 90% sequence identity, or at least 95% sequence identity, or at least 99% sequence identity to SEQ ID NO: 157.

In some embodiments, the MVA vector comprises a polynucleotide encoding an amino acid sequence of SEQ ID NO: 159 or having at least 90% sequence identity, or at least 95% sequence identity, or at least 99% sequence identity to SEQ ID NO: 159.

In some embodiments, the MVA vector comprises a polynucleotide encoding an amino acid sequence of SEQ ID NO: 161 or having at least 90% sequence identity, or at least 95% sequence identity, or at least 99% sequence identity to SEQ ID NO: 161.

In some embodiments, the MVA vector comprises a polynucleotide encoding an amino acid sequence of SEQ ID NO: 163 or having at least 90% sequence identity, or at least 95% sequence identity, or at least 99% sequence identity to SEQ ID NO: 163.

In some embodiments, the MVA vector comprises a polynucleotide encoding an amino acid sequence of SEQ ID NO: 165 or having at least 90% sequence identity, or at least 95% sequence identity, or at least 99% sequence identity to SEQ ID NO: 165.

In some embodiments, the MVA vector comprises a polynucleotide encoding an amino acid sequence of SEQ ID NO: 167 or having at least 90% sequence identity, or at least 95% sequence identity, or at least 99% sequence identity to SEQ ID NO: 167.

In some embodiments, the MVA vector comprises a polynucleotide encoding an amino acid sequence of SEQ ID NO: 169 or having at least 90% sequence identity, or at least 95% sequence identity, or at least 99% sequence identity to SEQ ID NO: 169.

In some embodiments, the MVA vector comprises a polynucleotide encoding an amino acid sequence of SEQ ID NO: 171 or having at least 90% sequence identity, or at least 95% sequence identity, or at least 99% sequence identity to SEQ ID NO: 171.

In some embodiments, the MVA vector comprises a polynucleotide encoding an amino acid sequence of SEQ ID NO: 173 or having at least 90% sequence identity, or at least 95% sequence identity, or at least 99% sequence identity to SEQ ID NO: 173.

In some embodiments, the MVA vector comprises a polynucleotide encoding an amino acid sequence of SEQ ID NO: 175 or having at least 90% sequence identity, or at least 95% sequence identity, or at least 99% sequence identity to SEQ ID NO: 175.

In some embodiments, the MVA vector comprises a polynucleotide encoding an amino acid sequence of SEQ ID NO: 177 or having at least 90% sequence identity, or at least 95% sequence identity, or at least 99% sequence identity to SEQ ID NO: 177.

In some embodiments, the MVA vector comprises a polynucleotide encoding an amino acid sequence of SEQ ID NO: 179 or having at least 90% sequence identity, or at least 95% sequence identity, or at least 99% sequence identity to SEQ ID NO: 179.

In some embodiments, the MVA vector comprises a polynucleotide encoding an amino acid sequence of SEQ ID NO: 181 or having at least 90% sequence identity, or at least 95% sequence identity, or at least 99% sequence identity to SEQ ID NO: 181.

In some embodiments, the MVA vector comprises a polynucleotide encoding an amino acid sequence of SEQ ID NO: 183 or having at least 90% sequence identity, or at least 95% sequence identity, or at least 99% sequence identity to SEQ ID NO: 183.

In some embodiments, the MVA vector comprises a polynucleotide encoding an amino acid sequence of SEQ ID NO: 185 or having at least 90% sequence identity, or at least 95% sequence identity, or at least 99% sequence identity to SEQ ID NO: 185.

In some embodiments, the MVA vector comprises a polynucleotide encoding an amino acid sequence of SEQ ID NO: 187 or having at least 90% sequence identity, or at least 95% sequence identity, or at least 99% sequence identity to SEQ ID NO: 187.

In some embodiments, the MVA vector comprises a polynucleotide encoding an amino acid sequence of SEQ ID NO: 189 or having at least 90% sequence identity, or at least 95% sequence identity, or at least 99% sequence identity to SEQ ID NO: 189.

In some embodiments, the MVA vector comprises a polynucleotide encoding an amino acid sequence of SEQ ID NO: 191 or having at least 90% sequence identity, or at least 95% sequence identity, or at least 99% sequence identity to SEQ ID NO: 191.

In some embodiments, the MVA vector comprises a polynucleotide encoding an amino acid sequence of SEQ ID NO: 193 or having at least 90% sequence identity, or at least 95% sequence identity, or at least 99% sequence identity to SEQ ID NO: 193.

In some embodiments, the MVA vector comprises a polynucleotide encoding an amino acid sequence of SEQ ID NO: 195 or having at least 90% sequence identity, or at least 95% sequence identity, or at least 99% sequence identity to SEQ ID NO: 195.

In some embodiments, the MVA vector comprises a polynucleotide encoding an amino acid sequence of SEQ ID NO: 197 or having at least 90% sequence identity, or at least 95% sequence identity, or at least 99% sequence identity to SEQ ID NO: 197.

In some embodiments, the MVA vector comprises a polynucleotide encoding an amino acid sequence of SEQ ID NO: 199 or having at least 90% sequence identity, or at least 95% sequence identity, or at least 99% sequence identity to SEQ ID NO: 199.

In some embodiments, the MVA vector comprises a polynucleotide encoding an amino acid sequence of SEQ ID NO: 201 or having at least 90% sequence identity, or at least 95% sequence identity, or at least 99% sequence identity to SEQ ID NO: 201.

In some embodiments, the MVA vector comprises a polynucleotide encoding an amino acid sequence of SEQ ID NO: 203 or having at least 90% sequence identity, or at least 95% sequence identity, or at least 99% sequence identity to SEQ ID NO: 203.

In some embodiments, the MVA vector comprises a polynucleotide encoding an amino acid sequence of SEQ ID NO: 205 or having at least 90% sequence identity, or at least 95% sequence identity, or at least 99% sequence identity to SEQ ID NO: 205.

In some embodiments, the MVA vector comprises a polynucleotide encoding an amino acid sequence of SEQ ID NO: 207 or having at least 90% sequence identity, or at least 95% sequence identity, or at least 99% sequence identity to SEQ ID NO: 207.

In some embodiments, the MVA vector comprises a polynucleotide encoding an amino acid sequence of SEQ ID NO: 209 or having at least 90% sequence identity, or at least 95% sequence identity, or at least 99% sequence identity to SEQ ID NO: 209.

In some embodiments, the MVA vector comprises a polynucleotide encoding an amino acid sequence of SEQ ID NO: 211 or having at least 90% sequence identity, or at least 95% sequence identity, or at least 99% sequence identity to SEQ ID NO: 211.

In some embodiments, the MVA vector comprises a polynucleotide encoding an amino acid sequence of SEQ ID NO: 213 or having at least 90% sequence identity, or at least 95% sequence identity, or at least 99% sequence identity to SEQ ID NO: 213.

In some embodiments, the MVA vector comprises a polynucleotide encoding an amino acid sequence of SEQ ID NO: 215 or having at least 90% sequence identity, or at least 95% sequence identity, or at least 99% sequence identity to SEQ ID NO: 215.

In some embodiments, the MVA vector comprises a polynucleotide encoding an amino acid sequence of SEQ ID NO: 217 or having at least 90% sequence identity, or at least 95% sequence identity, or at least 99% sequence identity to SEQ ID NO: 217.

In some embodiments, the MVA vector comprises a polynucleotide encoding an amino acid sequence of SEQ ID NO: 219 or having at least 90% sequence identity, or at least 95% sequence identity, or at least 99% sequence identity to SEQ ID NO: 219.

In some embodiments, the MVA vector comprises a polynucleotide encoding an amino acid sequence of SEQ ID NO: 221 or having at least 90% sequence identity, or at least 95% sequence identity, or at least 99% sequence identity to SEQ ID NO: 221.

In some embodiments, the MVA vector comprises a polynucleotide encoding an amino acid sequence of SEQ ID NO: 223 or having at least 90% sequence identity, or at least 95% sequence identity, or at least 99% sequence identity to SEQ ID NO: 223.

In some embodiments, the MVA vector comprises a polynucleotide encoding an amino acid sequence of SEQ ID NO: 225 or having at least 90% sequence identity, or at least 95% sequence identity, or at least 99% sequence identity to SEQ ID NO: 225.

In some embodiments, the MVA vector comprises a polynucleotide encoding an amino acid sequence of SEQ ID NO: 227 or having at least 90% sequence identity, or at least 95% sequence identity, or at least 99% sequence identity to SEQ ID NO: 227.

In some embodiments, the MVA vector comprises a polynucleotide encoding an amino acid sequence of SEQ ID NO: 229 or having at least 90% sequence identity, or at least 95% sequence identity, or at least 99% sequence identity to SEQ ID NO: 229.

In some embodiments, the MVA vector comprises a polynucleotide encoding an amino acid sequence of SEQ ID NO: 231 or having at least 90% sequence identity, or at least 95% sequence identity, or at least 99% sequence identity to SEQ ID NO: 231.

In some embodiments, the MVA vector comprises a polynucleotide encoding an amino acid sequence of SEQ ID NO: 233 or having at least 90% sequence identity, or at least 95% sequence identity, or at least 99% sequence identity to SEQ ID NO: 233.

In some embodiments, the MVA vector comprises a polynucleotide encoding an amino acid sequence of SEQ ID NO: 235 or having at least 90% sequence identity, or at least 95% sequence identity, or at least 99% sequence identity to SEQ ID NO: 235.

In some embodiments, the MVA vector comprises a polynucleotide encoding an amino acid sequence of SEQ ID NO: 237 or having at least 90% sequence identity, or at least 95% sequence identity, or at least 99% sequence identity to SEQ ID NO: 237.

In some embodiments, the MVA vector comprises a polynucleotide encoding an amino acid sequence of SEQ ID NO: 239 or having at least 90% sequence identity, or at least 95% sequence identity, or at least 99% sequence identity to SEQ ID NO: 239.

In some embodiments, the MVA vector comprises a polynucleotide encoding an amino acid sequence of SEQ ID NO: 241 or having at least 90% sequence identity, or at least 95% sequence identity, or at least 99% sequence identity to SEQ ID NO: 241.

In some embodiments, the MVA vector comprises a polynucleotide encoding an amino acid sequence of SEQ ID NO: 243 or having at least 90% sequence identity, or at least 95% sequence identity, or at least 99% sequence identity to SEQ ID NO: 243.

In some embodiments, the MVA vector comprises a polynucleotide encoding an amino acid sequence of SEQ ID NO: 245 or having at least 90% sequence identity, or at least 95% sequence identity, or at least 99% sequence identity to SEQ ID NO: 245.

In some embodiments, the MVA vector comprises a polynucleotide encoding an amino acid sequence of SEQ ID NO: 247 or having at least 90% sequence identity, or at least 95% sequence identity, or at least 99% sequence identity to SEQ ID NO: 247.

In some embodiments, the MVA vector comprises a polynucleotide encoding an amino acid sequence of SEQ ID NO: 249 or having at least 90% sequence identity, or at least 95% sequence identity, or at least 99% sequence identity to SEQ ID NO: 249.

In some embodiments, the MVA vector comprises a polynucleotide encoding an amino acid sequence of SEQ ID NO: 251 or having at least 90% sequence identity, or at least 95% sequence identity, or at least 99% sequence identity to SEQ ID NO: 251.

In some embodiments, the MVA vector comprises a polynucleotide encoding an amino acid sequence of SEQ ID NO: 253 or having at least 90% sequence identity, or at least 95% sequence identity, or at least 99% sequence identity to SEQ ID NO: 253.

In some embodiments, the MVA vector comprises a polynucleotide encoding an amino acid sequence of SEQ ID NO: 255 or having at least 90% sequence identity, or at least 95% sequence identity, or at least 99% sequence identity to SEQ ID NO: 255.

In some embodiments, the MVA vector comprises a polynucleotide encoding an amino acid sequence of SEQ ID NO: 257 or having at least 90% sequence identity, or at least 95% sequence identity, or at least 99% sequence identity to SEQ ID NO: 257.

In some embodiments, the MVA vector comprises a polynucleotide encoding an amino acid sequence of SEQ ID NO: 259 or having at least 90% sequence identity, or at least 95% sequence identity, or at least 99% sequence identity to SEQ ID NO: 259.

In some embodiments, the MVA vector comprises a polynucleotide encoding an amino acid sequence of SEQ ID NO: 261 or having at least 90% sequence identity, or at least 95% sequence identity, or at least 99% sequence identity to SEQ ID NO: 261.

In some embodiments, the MVA vector comprises a polynucleotide encoding an amino acid sequence of SEQ ID NO: 263 or having at least 90% sequence identity, or at least 95% sequence identity, or at least 99% sequence identity to SEQ ID NO: 263.

In some embodiments, the MVA vector comprises a polynucleotide encoding an amino acid sequence of SEQ ID NO: 265 or having at least 90% sequence identity, or at least 95% sequence identity, or at least 99% sequence identity to SEQ ID NO: 265.

In some embodiments, the MVA vector comprises a polynucleotide encoding an amino acid sequence of SEQ ID NO: 267 or having at least 90% sequence identity, or at least 95% sequence identity, or at least 99% sequence identity to SEQ ID NO: 267.

In some embodiments, the MVA vector comprises a polynucleotide encoding an amino acid sequence of SEQ ID NO: 269 or having at least 90% sequence identity, or at least 95% sequence identity, or at least 99% sequence identity to SEQ ID NO: 269.

In some embodiments, the MVA vector comprises a polynucleotide encoding an amino acid sequence of SEQ ID NO: 271 or having at least 90% sequence identity, or at least 95% sequence identity, or at least 99% sequence identity to SEQ ID NO: 271.

In some embodiments, the MVA vector comprises a polynucleotide encoding an amino acid sequence of SEQ ID NO: 273 or having at least 90% sequence identity, or at least 95% sequence identity, or at least 99% sequence identity to SEQ ID NO: 273.

In some embodiments, the MVA vector comprises a polynucleotide encoding an amino acid sequence of SEQ ID NO: 275 or having at least 90% sequence identity, or at least 95% sequence identity, or at least 99% sequence identity to SEQ ID NO: 275.

In some embodiments, the MVA vector comprises a polynucleotide encoding an amino acid sequence of SEQ ID NO: 277 or having at least 90% sequence identity, or at least 95% sequence identity, or at least 99% sequence identity to SEQ ID NO: 277.

In some embodiments, the MVA vector comprises a polynucleotide encoding an amino acid sequence of SEQ ID NO: 279 or having at least 90% sequence identity, or at least 95% sequence identity, or at least 99% sequence identity to SEQ ID NO: 279.

In some embodiments, the MVA vector comprises a polynucleotide encoding an amino acid sequence of SEQ ID NO: 281 or having at least 90% sequence identity, or at least 95% sequence identity, or at least 99% sequence identity to SEQ ID NO: 281.

In some embodiments, the MVA vector comprises a polynucleotide encoding an amino acid sequence of SEQ ID NO: 283 or having at least 90% sequence identity, or at least 95% sequence identity, or at least 99% sequence identity to SEQ ID NO: 283.

In some embodiments, the MVA vector comprises a polynucleotide encoding an amino acid sequence of SEQ ID NO: 285 or having at least 90% sequence identity, or at least 95% sequence identity, or at least 99% sequence identity to SEQ ID NO: 285.

In some embodiments, the MVA vector comprises a polynucleotide encoding an amino acid sequence of SEQ ID NO: 287 or having at least 90% sequence identity, or at least 95% sequence identity, or at least 99% sequence identity to SEQ ID NO: 287.

In some embodiments, the MVA vector comprises a polynucleotide encoding an amino acid sequence of SEQ ID NO: 289 or having at least 90% sequence identity, or at least 95% sequence identity, or at least 99% sequence identity to SEQ ID NO: 289.

In some embodiments, the MVA vector comprises a polynucleotide encoding an amino acid sequence of SEQ ID NO: 291 or having at least 90% sequence identity, or at least 95% sequence identity, or at least 99% sequence identity to SEQ ID NO: 291.

In some embodiments, the MVA vector comprises a polynucleotide encoding an amino acid sequence of SEQ ID NO: 293 or having at least 90% sequence identity, or at least 95% sequence identity, or at least 99% sequence identity to SEQ ID NO: 293.

In some embodiments, the MVA vector comprises a polynucleotide encoding an amino acid sequence of SEQ ID NO: 295 or having at least 90% sequence identity, or at least 95% sequence identity, or at least 99% sequence identity to SEQ ID NO: 295.

In some embodiments, the MVA vector comprises a polynucleotide encoding an amino acid sequence of SEQ ID NO: 297 or having at least 90% sequence identity, or at least 95% sequence identity, or at least 99% sequence identity to SEQ ID NO: 297.

In some embodiments, the MVA vector comprises a polynucleotide encoding an amino acid sequence of SEQ ID NO: 299 or having at least 90% sequence identity, or at least 95% sequence identity, or at least 99% sequence identity to SEQ ID NO: 299.

In some embodiments, the MVA vector comprises a polynucleotide encoding an amino acid sequence of SEQ ID NO: 301 or having at least 90% sequence identity, or at least 95% sequence identity, or at least 99% sequence identity to SEQ ID NO: 301.

In some embodiments, the MVA vector comprises a polynucleotide encoding an amino acid sequence of SEQ ID NO: 303 or having at least 90% sequence identity, or at least 95% sequence identity, or at least 99% sequence identity to SEQ ID NO: 303.

In some embodiments, the MVA vector comprises a polynucleotide encoding an amino acid sequence of SEQ ID NO: 305 or having at least 90% sequence identity, or at least 95% sequence identity, or at least 99% sequence identity to SEQ ID NO: 305.

In some embodiments, the MVA vector comprises a polynucleotide encoding an amino acid sequence of SEQ ID NO: 307 or having at least 90% sequence identity, or at least 95% sequence identity, or at least 99% sequence identity to SEQ ID NO: 307.

In some embodiments, the MVA vector comprises a polynucleotide encoding an amino acid sequence of SEQ ID NO: 309 or having at least 90% sequence identity, or at least 95% sequence identity, or at least 99% sequence identity to SEQ ID NO: 309.

In some embodiments, the MVA vector comprises a polynucleotide encoding an amino acid sequence of SEQ ID NO: 311 or having at least 90% sequence identity, or at least 95% sequence identity, or at least 99% sequence identity to SEQ ID NO: 311.

In some embodiments, the MVA vector comprises a polynucleotide encoding an amino acid sequence of SEQ ID NO: 313 or having at least 90% sequence identity, or at least 95% sequence identity, or at least 99% sequence identity to SEQ ID NO: 313.

In some embodiments, the MVA vector comprises a polynucleotide encoding an amino acid sequence of SEQ ID NO: 315 or having at least 90% sequence identity, or at least 95% sequence identity, or at least 99% sequence identity to SEQ ID NO: 315.

In some embodiments, the MVA vector comprises a polynucleotide encoding an amino acid sequence of SEQ ID NO: 317 or having at least 90% sequence identity, or at least 95% sequence identity, or at least 99% sequence identity to SEQ ID NO: 317.

In some embodiments, the MVA vector comprises a polynucleotide encoding an amino acid sequence of SEQ ID NO: 319 or having at least 90% sequence identity, or at least 95% sequence identity, or at least 99% sequence identity to SEQ ID NO: 319.

In some embodiments, the MVA vector comprises a polynucleotide encoding an amino acid sequence of SEQ ID NO: 321 or having at least 90% sequence identity, or at least 95% sequence identity, or at least 99% sequence identity to SEQ ID NO: 321.

In some embodiments, the MVA vector comprises a polynucleotide encoding an amino acid sequence of SEQ ID NO: 323 or having at least 90% sequence identity, or at least 95% sequence identity, or at least 99% sequence identity to SEQ ID NO: 323.

In some embodiments, the MVA vector comprises a polynucleotide encoding an amino acid sequence of SEQ ID NO: 325 or having at least 90% sequence identity, or at least 95% sequence identity, or at least 99% sequence identity to SEQ ID NO: 325.

In some embodiments, the MVA vector comprises a polynucleotide encoding an amino acid sequence of SEQ ID NO: 327 or having at least 90% sequence identity, or at least 95% sequence identity, or at least 99% sequence identity to SEQ ID NO: 327.

In some embodiments, the MVA vector comprises a polynucleotide encoding an amino acid sequence of SEQ ID NO: 329 or having at least 90% sequence identity, or at least 95% sequence identity, or at least 99% sequence identity to SEQ ID NO: 329.

In some embodiments, the MVA vector comprises a polynucleotide encoding an amino acid sequence of SEQ ID NO: 331 or having at least 90% sequence identity, or at least 95% sequence identity, or at least 99% sequence identity to SEQ ID NO: 331.

In some embodiments, the MVA vector comprises a polynucleotide encoding an amino acid sequence of SEQ ID NO: 333 or having at least 90% sequence identity, or at least 95% sequence identity, or at least 99% sequence identity to SEQ ID NO: 333.

In some embodiments, the MVA vector comprises a polynucleotide encoding an amino acid sequence of SEQ ID NO: 335 or having at least 90% sequence identity, or at least 95% sequence identity, or at least 99% sequence identity to SEQ ID NO: 335.

In some embodiments, the MVA vector comprises a polynucleotide encoding an amino acid sequence of SEQ ID NO: 337 or having at least 90% sequence identity, or at least 95% sequence identity, or at least 99% sequence identity to SEQ ID NO: 337.

In some embodiments, the MVA vector comprises a polynucleotide encoding an amino acid sequence of SEQ ID NO: 339 or having at least 90% sequence identity, or at least 95% sequence identity, or at least 99% sequence identity to SEQ ID NO: 339.

In some embodiments, the MVA vector comprises a polynucleotide encoding an amino acid sequence of SEQ ID NO: 341 or having at least 90% sequence identity, or at least 95% sequence identity, or at least 99% sequence identity to SEQ ID NO: 341.

In some embodiments, the MVA vector comprises a polynucleotide encoding an amino acid sequence of SEQ ID NO: 343 or having at least 90% sequence identity, or at least 95% sequence identity, or at least 99% sequence identity to SEQ ID NO: 343.

In some embodiments, the MVA vector comprises a polynucleotide encoding an amino acid sequence of SEQ ID NO: 345 or having at least 90% sequence identity, or at least 95% sequence identity, or at least 99% sequence identity to SEQ ID NO: 345.

In some embodiments, the MVA vector comprises a polynucleotide encoding an amino acid sequence of SEQ ID NO: 347 or having at least 90% sequence identity, or at least 95% sequence identity, or at least 99% sequence identity to SEQ ID NO: 347.

In some embodiments, the MVA vector comprises a polynucleotide encoding an amino acid sequence of SEQ ID NO: 349 or having at least 90% sequence identity, or at least 95% sequence identity, or at least 99% sequence identity to SEQ ID NO: 349.

In some embodiments, the MVA vector comprises a polynucleotide encoding an amino acid sequence of SEQ ID NO: 351 or having at least 90% sequence identity, or at least 95% sequence identity, or at least 99% sequence identity to SEQ ID NO: 351.

In some embodiments, the MVA vector comprises a polynucleotide encoding an amino acid sequence of SEQ ID NO: 353 or having at least 90% sequence identity, or at least 95% sequence identity, or at least 99% sequence identity to SEQ ID NO: 353.

In some embodiments, the MVA vector comprises a polynucleotide encoding an amino acid sequence of SEQ ID NO: 355 or having at least 90% sequence identity, or at least 95% sequence identity, or at least 99% sequence identity to SEQ ID NO: 355.

In some embodiments, the MVA vector comprises a polynucleotide encoding an amino acid sequence of SEQ ID NO: 357 or having at least 90% sequence identity, or at least 95% sequence identity, or at least 99% sequence identity to SEQ ID NO: 357.

In some embodiments, the MVA vector comprises a polynucleotide encoding an amino acid sequence of SEQ ID NO: 359 or having at least 90% sequence identity, or at least 95% sequence identity, or at least 99% sequence identity to SEQ ID NO: 359.

In some embodiments, the MVA vector comprises a polynucleotide encoding an amino acid sequence of SEQ ID NO: 361 or having at least 90% sequence identity, or at least 95% sequence identity, or at least 99% sequence identity to SEQ ID NO: 361.

In some embodiments, the MVA vector comprises a polynucleotide encoding an amino acid sequence of SEQ ID NO: 363 or having at least 90% sequence identity, or at least 95% sequence identity, or at least 99% sequence identity to SEQ ID NO: 363.

In some embodiments, the MVA vector comprises a polynucleotide encoding an amino acid sequence of SEQ ID NO: 365 or having at least 90% sequence identity, or at least 95% sequence identity, or at least 99% sequence identity to SEQ ID NO: 365.

In some embodiments, the MVA vector comprises a polynucleotide encoding an amino acid sequence of SEQ ID NO: 367 or having at least 90% sequence identity, or at least 95% sequence identity, or at least 99% sequence identity to SEQ ID NO: 367.

In some embodiments, the MVA vector comprises a polynucleotide encoding an amino acid sequence of SEQ ID NO: 369 or having at least 90% sequence identity, or at least 95% sequence identity, or at least 99% sequence identity to SEQ ID NO: 369.

In some embodiments, the MVA vector comprises a polynucleotide encoding an amino acid sequence of SEQ ID NO: 371 or having at least 90% sequence identity, or at least 95% sequence identity, or at least 99% sequence identity to SEQ ID NO: 371.

In some embodiments, the MVA vector comprises a polynucleotide encoding an amino acid sequence of SEQ ID NO: 373 or having at least 90% sequence identity, or at least 95% sequence identity, or at least 99% sequence identity to SEQ ID NO: 373.

In some embodiments, the MVA vector comprises a polynucleotide encoding an amino acid sequence of SEQ ID NO: 375 or having at least 90% sequence identity, or at least 95% sequence identity, or at least 99% sequence identity to SEQ ID NO: 375.

In some embodiments, the MVA vector comprises a polynucleotide encoding an amino acid sequence of SEQ ID NO: 377 or having at least 90% sequence identity, or at least 95% sequence identity, or at least 99% sequence identity to SEQ ID NO: 377.

In some embodiments, the MVA vector comprises a polynucleotide encoding an amino acid sequence of SEQ ID NO: 379 or having at least 90% sequence identity, or at least 95% sequence identity, or at least 99% sequence identity to SEQ ID NO: 379.

In some embodiments, the MVA vector comprises a polynucleotide encoding an amino acid sequence of SEQ ID NO: 381 or having at least 90% sequence identity, or at least 95% sequence identity, or at least 99% sequence identity to SEQ ID NO: 381.

In some embodiments, the MVA vector comprises a polynucleotide encoding an amino acid sequence of SEQ ID NO: 383 or having at least 90% sequence identity, or at least 95% sequence identity, or at least 99% sequence identity to SEQ ID NO: 383.

In some embodiments, the MVA vector comprises a polynucleotide encoding an amino acid sequence of SEQ ID NO: 385 or having at least 90% sequence identity, or at least 95% sequence identity, or at least 99% sequence identity to SEQ ID NO: 385.

In some embodiments, the MVA vector comprises a polynucleotide encoding an amino acid sequence of SEQ ID NO: 387 or having at least 90% sequence identity, or at least 95% sequence identity, or at least 99% sequence identity to SEQ ID NO: 387.

In some embodiments, the MVA vector comprises a polynucleotide encoding an amino acid sequence of SEQ ID NO: 389 or having at least 90% sequence identity, or at least 95% sequence identity, or at least 99% sequence identity to SEQ ID NO: 389.

In some embodiments, the MVA vector comprises a polynucleotide encoding an amino acid sequence of SEQ ID NO: 391 or having at least 90% sequence identity, or at least 95% sequence identity, or at least 99% sequence identity to SEQ ID NO: 391.

In some embodiments, the MVA vector comprises a polynucleotide encoding an amino acid sequence of SEQ ID NO: 393 or having at least 90% sequence identity, or at least 95% sequence identity, or at least 99% sequence identity to SEQ ID NO: 393.

In some embodiments, the MVA vector comprises a polynucleotide encoding an amino acid sequence of SEQ ID NO: 395 or having at least 90% sequence identity, or at least 95% sequence identity, or at least 99% sequence identity to SEQ ID NO: 395.

In some embodiments, the MVA vector comprises a polynucleotide encoding an amino acid sequence of SEQ ID NO: 397 or having at least 90% sequence identity, or at least 95% sequence identity, or at least 99% sequence identity to SEQ ID NO: 397.

In some embodiments, the MVA vector comprises a polynucleotide encoding an amino acid sequence of SEQ ID NO: 399 or having at least 90% sequence identity, or at least 95% sequence identity, or at least 99% sequence identity to SEQ ID NO: 399.

In some embodiments, the MVA vector comprises a polynucleotide encoding an amino acid sequence of SEQ ID NO: 401 or having at least 90% sequence identity, or at least 95% sequence identity, or at least 99% sequence identity to SEQ ID NO: 401.

In some embodiments, the MVA vector comprises a polynucleotide encoding an amino acid sequence of SEQ ID NO: 403 or having at least 90% sequence identity, or at least 95% sequence identity, or at least 99% sequence identity to SEQ ID NO: 403.

In some embodiments, the MVA vector comprises a polynucleotide encoding an amino acid sequence of SEQ ID NO: 405 or having at least 90% sequence identity, or at least 95% sequence identity, or at least 99% sequence identity to SEQ ID NO: 405.

In some embodiments, the MVA vector comprises a polynucleotide encoding an amino acid sequence of SEQ ID NO: 405 or having at least 90% sequence identity, or at least 95% sequence identity, or at least 99% sequence identity to SEQ ID NO: 407.

In some embodiments, the MVA vector comprises a polynucleotide encoding an amino acid sequence of SEQ ID NO: 405 or having at least 90% sequence identity, or at least 95% sequence identity, or at least 99% sequence identity to SEQ ID NO: 421.

In some embodiments, the MVA vector comprises a polynucleotide encoding an amino acid sequence of two or more of the polypeptides selected from SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, and 421, and fragments thereof.

### Self-Replicating RNA Molecules

In some embodiments, the viral vector is a self-replicating RMA molecule derived from an alphavirus.

Self-replicating RNA molecules may be derived from alphavirus. Alphaviruses may belong to the VEEV/EEEV group, or the SF group, or the SIN group. Non-limiting examples of SF group alphaviruses include Semliki Forest virus, O′Nyong-Nyong virus, Ross River virus, Middelburg virus, Chikungunya virus, Barmah Forest virus, Getah virus, Mayaro virus, Sagiyama virus, Bebaru virus, and Una virus. Non-limiting examples of SIN group alphaviruses include Sindbis virus, Girdwood S. A. virus, South African Arbovirus No. 86, Ockelbo virus, Aura virus, Babanki virus, Whataroa virus, and Kyzylagach virus. Non-limiting examples of VEEV/EEEV group alphaviruses include Eastern equine encephalitis virus (EEEV), Venezuelan equine encephalitis virus (VEEV), Everglades virus (EVEV), Mucambo virus (MUCV), Pixuna virus (PIXV), Middleburg virus (MIDV), Chikungunya virus (CHIKV), O′Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), and Una virus (UNAV).

The self-replicating RNA molecules can be derived from alphavirus genomes, meaning that they have some of the structural characteristics of alphavirus genomes, or similar to them. The self-replicating RNA molecules can be derived from modified alphavirus genomes.

Self-replicating RNA molecules may be derived from Eastern equine encephalitis virus (EEEV), Venezuelan equine encephalitis virus (VEEV), Everglades virus (EVEV), Mucambo virus (MUCV), Semliki forest virus (SFV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu (NDUV), and Buggy Creek virus. Virulent and avirulent alphavirus strains are both suitable. In some embodiments, the alphavirus RNA replicon is of a Sindbis virus (SIN), a Semliki Forest virus (SFV), a Ross River virus (RRV), a Venezuelan equine encephalitis virus (VEEV), or an Eastern equine encephalitis virus (EEEV).

In some embodiments, the alphavirus-derived self-replicating RNA molecule is a Venezuelan equine encephalitis virus (VEEV).

The self-replicating RNA molecules can contain RNA sequences from (or amino acid sequences encoded by) a wild-type New World or Old World alphavirus genome. Any of the self-replicating RNA molecules disclosed herein can contain RNA sequences “derived from” or “based on” wild type alphavirus genome sequences, meaning that they have at least 60% or at least 65% or at least 68% or at least 70% or at least 80% or at least 85% or at least 90% or at least 95% or at least 97% or at least 98% or at least 99% or 100% or 80-99% or 90-100% or 95-99% or 95-100% or 97-99% or 98-99% sequence identity with an RNA sequence (which can be a corresponding RNA sequence) from a wild type RNA alphavirus genome, which can be a New World or Old World alphavirus genome.

Self-replicating RNA molecules contain all of the genetic information required for directing their own amplification or self-replication within a permissive cell. To direct their own replication, self-replicating RNA molecules encode polymerase, replicase, or other proteins which may interact with viral or host cell-derived proteins, nucleic acids or ribonucleoproteins to catalyze the RNA amplification process; and contain cis-acting RNA sequences required for replication and transcription of the replicon-encoded RNA. Thus, RNA replication leads to the production of multiple daughter RNAs. These daughter RNAs, as well as collinear subgenomic transcripts, can be translated to provide in situ expression of a gene of interest, or can be transcribed to provide further transcripts with the same sense as the delivered RNA which are translated to provide in situ expression of the gene of interest. The overall results of this sequence of transcriptions is a huge amplification in the number of the introduced replicon RNAs and so the encoded gene of interest becomes a major polypeptide product of the cells.

There are two open reading frames (ORF's) in the genome of alphaviruses, non-structural (ns) and structural genes. The ns ORF encodes proteins (nsP1-nsP4) necessary for transcription and replication of viral RNA and are produced as a polyprotein and are the virus replication machinery. The structural ORF encodes three structural proteins: the core nucleocapsid protein C, and the envelope proteins P62 and E1 that associate as a heterodimer. The viral membrane-anchored surface glycoproteins are responsible for receptor recognition and entry into target cells through membrane fusion. The four ns protein genes are encoded by genes in the 5′ two-thirds of the genome, while the three structural proteins are translated from a subgenomic mRNA colinear with the 3′ one-third of the genome.

Self-replicating RNA molecules can be used as basis of introducing foreign sequences to host cells by replacing viral sequences encoding structural genes or inserting the foreign sequences 5′ or 3′ of the sequences encoding the structural genes. They can be engineered to replace the viral structural genes downstream of the replicase, which are under control of a subgenomic promoter, by genes of interest (GOI), e.g. any of the polynucleotides encoding for any of the polypeptides of the disclosure. Upon transfection, the replicase which is translated immediately, interacts with the 5′ and 3′ termini of the genomic RNA, and synthesizes complementary genomic RNA copies. Those act as templates for the synthesis of novel positive-stranded, capped, and poly-adenylated genomic copies, and subgenomic transcripts. Amplification eventually leads to very high RNA copy numbers of up to 2×10⁵ copies per cell. The result is a uniform and/or enhanced expression of a GOI (e.g. a polynucleotide encoding for one or more of the polypeptides of the disclosure) that can affect vaccine efficacy or therapeutic impact of a treatment. Vaccines based on self-replicating RNA molecules can therefore be dosed at very low levels due to the very high copies of RNA generated compared to conventional viral vector.

The self-replicating RNA molecules of the disclosure comprising the RNA encoding for one or more of the multiple myeloma neoantigens polypeptides of the disclosure may be utilized as therapeutics by delivering them to a subject having multiple myeloma or at risk of multiple myeloma using various technologies, including viral vectors as described herein or other delivery technologies as also described herein.

The multiple myeloma cancer neoantigen polynucleotides of the disclosure can be expressed under the control of a subgenomic promoter. In certain embodiments, instead of the native subgenomic promoter, the subgenomic RNA can be placed under control of internal ribosome entry site (IRES) derived from encephalomyocarditis viruses (EMCV), Bovine Viral Diarrhea Viruses (BVDV), polioviruses, Foot-and-mouth disease viruses (FMD), enterovirus 71, or hepatitis C viruses. Subgenomic promoters range from 24 nucleotide (Sindbis virus) to over 100 nucleotides (Beet necrotic yellow vein virus) and are usually found upstream of the transcription start.

The disclosure provides a self-replicating RNA molecule containing all of the genetic information required for directing its own amplification or self-replication within a permissive cell.

The disclosure also provides a self-replicating RNA molecule that can be used as the basis of introducing foreign sequences to host cells (e.g. the multiple myeloma neoantigen polypeptides of the disclosure) by replacing viral sequences encoding structural genes. Provided herein is a viral vector comprising any of the polynucleotides of the disclosure, wherein the vector is a self-replicating RNA molecule.

In some embodiments, the self-replicating RNA molecule comprises an RNA sequence encoding an amino acid sequence of SEQ ID NO: 1 or having at least 90% sequence identity, or at least 95% sequence identity, or at least 99% sequence identity to SEQ ID NO: 1.

In some embodiments, the self-replicating RNA molecule comprises a polynucleotide encoding an amino acid sequence of SEQ ID NO: 3 or having at least 90% sequence identity, or at least 95% sequence identity, or at least 99% sequence identity to SEQ ID NO: 3.

In some embodiments, the self-replicating RNA molecule comprises a polynucleotide encoding an amino acid sequence of SEQ ID NO: 5 or having at least 90% sequence identity, or at least 95% sequence identity, or at least 99% sequence identity to SEQ ID NO: 5.

In some embodiments, the self-replicating RNA molecule comprises a polynucleotide encoding an amino acid sequence of SEQ ID NO: 7 or having at least 90% sequence identity, or at least 95% sequence identity, or at least 99% sequence identity to SEQ ID NO: 7.

In some embodiments, the self-replicating RNA molecule comprises a polynucleotide encoding an amino acid sequence of SEQ ID NO: 9 or having at least 90% sequence identity, or at least 95% sequence identity, or at least 99% sequence identity to SEQ ID NO: 9.

In some embodiments, the self-replicating RNA molecule comprises a polynucleotide encoding an amino acid sequence of SEQ ID NO: 11 or having at least 90% sequence identity, or at least 95% sequence identity, or at least 99% sequence identity to SEQ ID NO: 11.

In some embodiments, the self-replicating RNA molecule comprises a polynucleotide encoding an amino acid sequence of SEQ ID NO: 13 or having at least 90% sequence identity, or at least 95% sequence identity, or at least 99% sequence identity to SEQ ID NO: 13.

In some embodiments, the self-replicating RNA molecule comprises a polynucleotide encoding an amino acid sequence of SEQ ID NO: 15 or having at least 90% sequence identity, or at least 95% sequence identity, or at least 99% sequence identity to SEQ ID NO: 15.

In some embodiments, the self-replicating RNA molecule comprises a polynucleotide encoding an amino acid sequence of SEQ ID NO: 17 or having at least 90% sequence identity, or at least 95% sequence identity, or at least 99% sequence identity to SEQ ID NO: 17.

In some embodiments, the self-replicating RNA molecule comprises a polynucleotide encoding an amino acid sequence of SEQ ID NO: 19 or having at least 90% sequence identity, or at least 95% sequence identity, or at least 99% sequence identity to SEQ ID NO: 19.

In some embodiments, the self-replicating RNA molecule comprises a polynucleotide encoding an amino acid sequence of SEQ ID NO: 21 or having at least 90% sequence identity, or at least 95% sequence identity, or at least 99% sequence identity to SEQ ID NO: 21.

In some embodiments, the self-replicating RNA molecule comprises a polynucleotide encoding an amino acid sequence of SEQ ID NO: 23 or having at least 90% sequence identity, or at least 95% sequence identity, or at least 99% sequence identity to SEQ ID NO: 23.

In some embodiments, the self-replicating RNA molecule comprises a polynucleotide encoding an amino acid sequence of SEQ ID NO: 25 or having at least 90% sequence identity, or at least 95% sequence identity, or at least 99% sequence identity to SEQ ID NO: 25.

In some embodiments, the self-replicating RNA molecule comprises a polynucleotide encoding an amino acid sequence of SEQ ID NO: 27 or having at least 90% sequence identity, or at least 95% sequence identity, or at least 99% sequence identity to SEQ ID NO: 27.

In some embodiments, the self-replicating RNA molecule comprises a polynucleotide encoding an amino acid sequence of SEQ ID NO: 29 or having at least 90% sequence identity, or at least 95% sequence identity, or at least 99% sequence identity to SEQ ID NO: 29.

In some embodiments, the self-replicating RNA molecule comprises a polynucleotide encoding an amino acid sequence of SEQ ID NO: 31 or having at least 90% sequence identity, or at least 95% sequence identity, or at least 99% sequence identity to SEQ ID NO: 31.

In some embodiments, the self-replicating RNA molecule comprises a polynucleotide encoding an amino acid sequence of SEQ ID NO: 33 or having at least 90% sequence identity, or at least 95% sequence identity, or at least 99% sequence identity to SEQ ID NO: 33.

In some embodiments, the self-replicating RNA molecule comprises a polynucleotide encoding an amino acid sequence of SEQ ID NO: 35 or having at least 90% sequence identity, or at least 95% sequence identity, or at least 99% sequence identity to SEQ ID NO: 35.

In some embodiments, the self-replicating RNA molecule comprises a polynucleotide encoding an amino acid sequence of SEQ ID NO: 37 or having at least 90% sequence identity, or at least 95% sequence identity, or at least 99% sequence identity to SEQ ID NO: 37.

In some embodiments, the self-replicating RNA molecule comprises a polynucleotide encoding an amino acid sequence of SEQ ID NO: 39 or having at least 90% sequence identity, or at least 95% sequence identity, or at least 99% sequence identity to SEQ ID NO: 39.

In some embodiments, the self-replicating RNA molecule comprises a polynucleotide encoding an amino acid sequence of SEQ ID NO: 41 or having at least 90% sequence identity, or at least 95% sequence identity, or at least 99% sequence identity to SEQ ID NO: 41.

In some embodiments, the self-replicating RNA molecule comprises a polynucleotide encoding an amino acid sequence of SEQ ID NO: 43 or having at least 90% sequence identity, or at least 95% sequence identity, or at least 99% sequence identity to SEQ ID NO: 43.

In some embodiments, the self-replicating RNA molecule comprises a polynucleotide encoding an amino acid sequence of SEQ ID NO: 45 or having at least 90% sequence identity, or at least 95% sequence identity, or at least 99% sequence identity to SEQ ID NO: 45.

In some embodiments, the self-replicating RNA molecule comprises a polynucleotide encoding an amino acid sequence of SEQ ID NO: 47 or having at least 90% sequence identity, or at least 95% sequence identity, or at least 99% sequence identity to SEQ ID NO: 47.

In some embodiments, the self-replicating RNA molecule comprises a polynucleotide encoding an amino acid sequence of SEQ ID NO: 49 or having at least 90% sequence identity, or at least 95% sequence identity, or at least 99% sequence identity to SEQ ID NO: 49. In some embodiments, the self-replicating RNA molecule comprises a polynucleotide encoding an amino acid sequence of SEQ ID NO: 51 or having at least 90% sequence identity, or at least 95% sequence identity, or at least 99% sequence identity to SEQ ID NO: 51.

In some embodiments, the self-replicating RNA molecule comprises a polynucleotide encoding an amino acid sequence of SEQ ID NO: 53 or having at least 90% sequence identity, or at least 95% sequence identity, or at least 99% sequence identity to SEQ ID NO: 53.

In some embodiments, the self-replicating RNA molecule comprises a polynucleotide encoding an amino acid sequence of SEQ ID NO: 55 or having at least 90% sequence identity, or at least 95% sequence identity, or at least 99% sequence identity to SEQ ID NO: 55.

In some embodiments, the self-replicating RNA molecule comprises a polynucleotide encoding an amino acid sequence of SEQ ID NO: 57 or having at least 90% sequence identity, or at least 95% sequence identity, or at least 99% sequence identity to SEQ ID NO: 57.

In some embodiments, the self-replicating RNA molecule comprises a polynucleotide encoding an amino acid sequence of SEQ ID NO: 59 or having at least 90% sequence identity, or at least 95% sequence identity, or at least 99% sequence identity to SEQ ID NO: 59.

In some embodiments, the self-replicating RNA molecule comprises a polynucleotide encoding an amino acid sequence of SEQ ID NO: 61 or having at least 90% sequence identity, or at least 95% sequence identity, or at least 99% sequence identity to SEQ ID NO: 61.

In some embodiments, the self-replicating RNA molecule comprises a polynucleotide encoding an amino acid sequence of SEQ ID NO: 63 or having at least 90% sequence identity, or at least 95% sequence identity, or at least 99% sequence identity to SEQ ID NO: 63.

In some embodiments, the self-replicating RNA molecule comprises a polynucleotide encoding an amino acid sequence of SEQ ID NO: 65 or having at least 90% sequence identity, or at least 95% sequence identity, or at least 99% sequence identity to SEQ ID NO: 65.

In some embodiments, the self-replicating RNA molecule comprises a polynucleotide encoding an amino acid sequence of SEQ ID NO: 67 or having at least 90% sequence identity, or at least 95% sequence identity, or at least 99% sequence identity to SEQ ID NO: 67.

In some embodiments, the self-replicating RNA molecule comprises a polynucleotide encoding an amino acid sequence of SEQ ID NO: 69 or having at least 90% sequence identity, or at least 95% sequence identity, or at least 99% sequence identity to SEQ ID NO: 69.

In some embodiments, the self-replicating RNA molecule comprises a polynucleotide encoding an amino acid sequence of SEQ ID NO: 71 or having at least 90% sequence identity, or at least 95% sequence identity, or at least 99% sequence identity to SEQ ID NO: 71.

In some embodiments, the self-replicating RNA molecule comprises a polynucleotide encoding an amino acid sequence of SEQ ID NO: 73 or having at least 90% sequence identity, or at least 95% sequence identity, or at least 99% sequence identity to SEQ ID NO: 73.

In some embodiments, the self-replicating RNA molecule comprises a polynucleotide encoding an amino acid sequence of SEQ ID NO: 75 or having at least 90% sequence identity, or at least 95% sequence identity, or at least 99% sequence identity to SEQ ID NO: 75.

In some embodiments, the self-replicating RNA molecule comprises a polynucleotide encoding an amino acid sequence of SEQ ID NO: 77 or having at least 90% sequence identity, or at least 95% sequence identity, or at least 99% sequence identity to SEQ ID NO: 77.

In some embodiments, the self-replicating RNA molecule comprises a polynucleotide encoding an amino acid sequence of SEQ ID NO: 79 or having at least 90% sequence identity, or at least 95% sequence identity, or at least 99% sequence identity to SEQ ID NO: 79.

In some embodiments, the self-replicating RNA molecule comprises a polynucleotide encoding an amino acid sequence of SEQ ID NO: 81 or having at least 90% sequence identity, or at least 95% sequence identity, or at least 99% sequence identity to SEQ ID NO: 81.

In some embodiments, the self-replicating RNA molecule comprises a polynucleotide encoding an amino acid sequence of SEQ ID NO: 83 or having at least 90% sequence identity, or at least 95% sequence identity, or at least 99% sequence identity to SEQ ID NO: 83.

In some embodiments, the self-replicating RNA molecule comprises a polynucleotide encoding an amino acid sequence of SEQ ID NO: 85 or having at least 90% sequence identity, or at least 95% sequence identity, or at least 99% sequence identity to SEQ ID NO: 85.

In some embodiments, the self-replicating RNA molecule comprises a polynucleotide encoding an amino acid sequence of SEQ ID NO: 87 or having at least 90% sequence identity, or at least 95% sequence identity, or at least 99% sequence identity to SEQ ID NO: 87.

In some embodiments, the self-replicating RNA molecule comprises a polynucleotide encoding an amino acid sequence of SEQ ID NO: 89 or having at least 90% sequence identity, or at least 95% sequence identity, or at least 99% sequence identity to SEQ ID NO: 89.

In some embodiments, the self-replicating RNA molecule comprises a polynucleotide encoding an amino acid sequence of SEQ ID NO: 91 or having at least 90% sequence identity, or at least 95% sequence identity, or at least 99% sequence identity to SEQ ID NO: 91.

In some embodiments, the self-replicating RNA molecule comprises a polynucleotide encoding an amino acid sequence of SEQ ID NO: 93 or having at least 90% sequence identity, or at least 95% sequence identity, or at least 99% sequence identity to SEQ ID NO: 93.

In some embodiments, the self-replicating RNA molecule comprises a polynucleotide encoding an amino acid sequence of SEQ ID NO: 95 or having at least 90% sequence identity, or at least 95% sequence identity, or at least 99% sequence identity to SEQ ID NO: 95.

In some embodiments, the self-replicating RNA molecule comprises a polynucleotide encoding an amino acid sequence of SEQ ID NO: 97 or having at least 90% sequence identity, or at least 95% sequence identity, or at least 99% sequence identity to SEQ ID NO: 97.

In some embodiments, the self-replicating RNA molecule comprises a polynucleotide encoding an amino acid sequence of SEQ ID NO: 99 or having at least 90% sequence identity, or at least 95% sequence identity, or at least 99% sequence identity to SEQ ID NO: 99.

In some embodiments, the self-replicating RNA molecule comprises a polynucleotide encoding an amino acid sequence of SEQ ID NO: 101 or having at least 90% sequence identity, or at least 95% sequence identity, or at least 99% sequence identity to SEQ ID NO: 101.

In some embodiments, the self-replicating RNA molecule comprises a polynucleotide encoding an amino acid sequence of SEQ ID NO: 103 or having at least 90% sequence identity, or at least 95% sequence identity, or at least 99% sequence identity to SEQ ID NO: 103.

In some embodiments, the self-replicating RNA molecule comprises a polynucleotide encoding an amino acid sequence of SEQ ID NO: 105 or having at least 90% sequence identity, or at least 95% sequence identity, or at least 99% sequence identity to SEQ ID NO: 105.

In some embodiments, the self-replicating RNA molecule comprises a polynucleotide encoding an amino acid sequence of SEQ ID NO: 107 or having at least 90% sequence identity, or at least 95% sequence identity, or at least 99% sequence identity to SEQ ID NO: 107.

In some embodiments, the self-replicating RNA molecule comprises a polynucleotide encoding an amino acid sequence of SEQ ID NO: 109 or having at least 90% sequence identity, or at least 95% sequence identity, or at least 99% sequence identity to SEQ ID NO: 109.

In some embodiments, the self-replicating RNA molecule comprises a polynucleotide encoding an amino acid sequence of SEQ ID NO: 111 or having at least 90% sequence identity, or at least 95% sequence identity, or at least 99% sequence identity to SEQ ID NO: 111.

In some embodiments, the self-replicating RNA molecule comprises a polynucleotide encoding an amino acid sequence of SEQ ID NO: 113 or having at least 90% sequence identity, or at least 95% sequence identity, or at least 99% sequence identity to SEQ ID NO: 113.

In some embodiments, the self-replicating RNA molecule comprises a polynucleotide encoding an amino acid sequence of SEQ ID NO: 115 or having at least 90% sequence identity, or at least 95% sequence identity, or at least 99% sequence identity to SEQ ID NO: 115.

In some embodiments, the self-replicating RNA molecule comprises a polynucleotide encoding an amino acid sequence of SEQ ID NO: 117 or having at least 90% sequence identity, or at least 95% sequence identity, or at least 99% sequence identity to SEQ ID NO: 117.

In some embodiments, the self-replicating RNA molecule comprises a polynucleotide encoding an amino acid sequence of SEQ ID NO: 119 or having at least 90% sequence identity, or at least 95% sequence identity, or at least 99% sequence identity to SEQ ID NO: 119.

In some embodiments, the self-replicating RNA molecule comprises a polynucleotide encoding an amino acid sequence of SEQ ID NO: 121 or having at least 90% sequence identity, or at least 95% sequence identity, or at least 99% sequence identity to SEQ ID NO: 121.

In some embodiments, the self-replicating RNA molecule comprises a polynucleotide encoding an amino acid sequence of SEQ ID NO: 123 or having at least 90% sequence identity, or at least 95% sequence identity, or at least 99% sequence identity to SEQ ID NO: 123.

In some embodiments, the self-replicating RNA molecule comprises a polynucleotide encoding an amino acid sequence of SEQ ID NO: 125 or having at least 90% sequence identity, or at least 95% sequence identity, or at least 99% sequence identity to SEQ ID NO: 125.

In some embodiments, the self-replicating RNA molecule comprises a polynucleotide encoding an amino acid sequence of SEQ ID NO: 127 or having at least 90% sequence identity, or at least 95% sequence identity, or at least 99% sequence identity to SEQ ID NO: 127.

In some embodiments, the self-replicating RNA molecule comprises a polynucleotide encoding an amino acid sequence of SEQ ID NO: 129 or having at least 90% sequence identity, or at least 95% sequence identity, or at least 99% sequence identity to SEQ ID NO: 129.

In some embodiments, the self-replicating RNA molecule comprises a polynucleotide encoding an amino acid sequence of SEQ ID NO: 131 or having at least 90% sequence identity, or at least 95% sequence identity, or at least 99% sequence identity to SEQ ID NO: 131.

In some embodiments, the self-replicating RNA molecule comprises a polynucleotide encoding an amino acid sequence of SEQ ID NO: 133 or having at least 90% sequence identity, or at least 95% sequence identity, or at least 99% sequence identity to SEQ ID NO: 133.

In some embodiments, the self-replicating RNA molecule comprises a polynucleotide encoding an amino acid sequence of SEQ ID NO: 135 or having at least 90% sequence identity, or at least 95% sequence identity, or at least 99% sequence identity to SEQ ID NO: 135.

In some embodiments, the self-replicating RNA molecule comprises a polynucleotide encoding an amino acid sequence of SEQ ID NO: 137 or having at least 90% sequence identity, or at least 95% sequence identity, or at least 99% sequence identity to SEQ ID NO: 137.

In some embodiments, the self-replicating RNA molecule comprises a polynucleotide encoding an amino acid sequence of SEQ ID NO: 139 or having at least 90% sequence identity, or at least 95% sequence identity, or at least 99% sequence identity to SEQ ID NO: 139.

In some embodiments, the self-replicating RNA molecule comprises a polynucleotide encoding an amino acid sequence of SEQ ID NO: 141 or having at least 90% sequence identity, or at least 95% sequence identity, or at least 99% sequence identity to SEQ ID NO: 141.

In some embodiments, the self-replicating RNA molecule comprises a polynucleotide encoding an amino acid sequence of SEQ ID NO: 143 or having at least 90% sequence identity, or at least 95% sequence identity, or at least 99% sequence identity to SEQ ID NO: 143.

In some embodiments, the self-replicating RNA molecule comprises a polynucleotide encoding an amino acid sequence of SEQ ID NO: 145 or having at least 90% sequence identity, or at least 95% sequence identity, or at least 99% sequence identity to SEQ ID NO: 145.

In some embodiments, the self-replicating RNA molecule comprises a polynucleotide encoding an amino acid sequence of SEQ ID NO: 147 or having at least 90% sequence identity, or at least 95% sequence identity, or at least 99% sequence identity to SEQ ID NO: 147.

In some embodiments, the self-replicating RNA molecule comprises a polynucleotide encoding an amino acid sequence of SEQ ID NO: 149 or having at least 90% sequence identity, or at least 95% sequence identity, or at least 99% sequence identity to SEQ ID NO: 149.

In some embodiments, the self-replicating RNA molecule comprises a polynucleotide encoding an amino acid sequence of SEQ ID NO: 151 or having at least 90% sequence identity, or at least 95% sequence identity, or at least 99% sequence identity to SEQ ID NO: 151.

In some embodiments, the self-replicating RNA molecule comprises a polynucleotide encoding an amino acid sequence of SEQ ID NO: 153 or having at least 90% sequence identity, or at least 95% sequence identity, or at least 99% sequence identity to SEQ ID NO: 153.

In some embodiments, the self-replicating RNA molecule comprises a polynucleotide encoding an amino acid sequence of SEQ ID NO: 155 or having at least 90% sequence identity, or at least 95% sequence identity, or at least 99% sequence identity to SEQ ID NO: 155.

In some embodiments, the self-replicating RNA molecule comprises a polynucleotide encoding an amino acid sequence of SEQ ID NO: 157 or having at least 90% sequence identity, or at least 95% sequence identity, or at least 99% sequence identity to SEQ ID NO: 157.

In some embodiments, the self-replicating RNA molecule comprises a polynucleotide encoding an amino acid sequence of SEQ ID NO: 159 or having at least 90% sequence identity, or at least 95% sequence identity, or at least 99% sequence identity to SEQ ID NO: 159.

In some embodiments, the self-replicating RNA molecule comprises a polynucleotide encoding an amino acid sequence of SEQ ID NO: 161 or having at least 90% sequence identity, or at least 95% sequence identity, or at least 99% sequence identity to SEQ ID NO: 161.

In some embodiments, the self-replicating RNA molecule comprises a polynucleotide encoding an amino acid sequence of SEQ ID NO: 163 or having at least 90% sequence identity, or at least 95% sequence identity, or at least 99% sequence identity to SEQ ID NO: 163.

In some embodiments, the self-replicating RNA molecule comprises a polynucleotide encoding an amino acid sequence of SEQ ID NO: 165 or having at least 90% sequence identity, or at least 95% sequence identity, or at least 99% sequence identity to SEQ ID NO: 165.

In some embodiments, the self-replicating RNA molecule comprises a polynucleotide encoding an amino acid sequence of SEQ ID NO: 167 or having at least 90% sequence identity, or at least 95% sequence identity, or at least 99% sequence identity to SEQ ID NO: 167.

In some embodiments, the self-replicating RNA molecule comprises a polynucleotide encoding an amino acid sequence of SEQ ID NO: 169 or having at least 90% sequence identity, or at least 95% sequence identity, or at least 99% sequence identity to SEQ ID NO: 169.

In some embodiments, the self-replicating RNA molecule comprises a polynucleotide encoding an amino acid sequence of SEQ ID NO: 171 or having at least 90% sequence identity, or at least 95% sequence identity, or at least 99% sequence identity to SEQ ID NO: 171.

In some embodiments, the self-replicating RNA molecule comprises a polynucleotide encoding an amino acid sequence of SEQ ID NO: 173 or having at least 90% sequence identity, or at least 95% sequence identity, or at least 99% sequence identity to SEQ ID NO: 173.

In some embodiments, the self-replicating RNA molecule comprises a polynucleotide encoding an amino acid sequence of SEQ ID NO: 175 or having at least 90% sequence identity, or at least 95% sequence identity, or at least 99% sequence identity to SEQ ID NO: 175.

In some embodiments, the self-replicating RNA molecule comprises a polynucleotide encoding an amino acid sequence of SEQ ID NO: 177 or having at least 90% sequence identity, or at least 95% sequence identity, or at least 99% sequence identity to SEQ ID NO: 177.

In some embodiments, the self-replicating RNA molecule comprises a polynucleotide encoding an amino acid sequence of SEQ ID NO: 179 or having at least 90% sequence identity, or at least 95% sequence identity, or at least 99% sequence identity to SEQ ID NO: 179.

In some embodiments, the self-replicating RNA molecule comprises a polynucleotide encoding an amino acid sequence of SEQ ID NO: 181 or having at least 90% sequence identity, or at least 95% sequence identity, or at least 99% sequence identity to SEQ ID NO: 181.

In some embodiments, the self-replicating RNA molecule comprises a polynucleotide encoding an amino acid sequence of SEQ ID NO: 183 or having at least 90% sequence identity, or at least 95% sequence identity, or at least 99% sequence identity to SEQ ID NO: 183.

In some embodiments, the self-replicating RNA molecule comprises a polynucleotide encoding an amino acid sequence of SEQ ID NO: 185 or having at least 90% sequence identity, or at least 95% sequence identity, or at least 99% sequence identity to SEQ ID NO: 185.

In some embodiments, the self-replicating RNA molecule comprises a polynucleotide encoding an amino acid sequence of SEQ ID NO: 187 or having at least 90% sequence identity, or at least 95% sequence identity, or at least 99% sequence identity to SEQ ID NO: 187.

In some embodiments, the self-replicating RNA molecule comprises a polynucleotide encoding an amino acid sequence of SEQ ID NO: 189 or having at least 90% sequence identity, or at least 95% sequence identity, or at least 99% sequence identity to SEQ ID NO: 189.

In some embodiments, the self-replicating RNA molecule comprises a polynucleotide encoding an amino acid sequence of SEQ ID NO: 191 or having at least 90% sequence identity, or at least 95% sequence identity, or at least 99% sequence identity to SEQ ID NO: 191.

In some embodiments, the self-replicating RNA molecule comprises a polynucleotide encoding an amino acid sequence of SEQ ID NO: 193 or having at least 90% sequence identity, or at least 95% sequence identity, or at least 99% sequence identity to SEQ ID NO: 193.

In some embodiments, the self-replicating RNA molecule comprises a polynucleotide encoding an amino acid sequence of SEQ ID NO: 195 or having at least 90% sequence identity, or at least 95% sequence identity, or at least 99% sequence identity to SEQ ID NO: 195.

In some embodiments, the self-replicating RNA molecule comprises a polynucleotide encoding an amino acid sequence of SEQ ID NO: 197 or having at least 90% sequence identity, or at least 95% sequence identity, or at least 99% sequence identity to SEQ ID NO: 197.

In some embodiments, the self-replicating RNA molecule comprises a polynucleotide encoding an amino acid sequence of SEQ ID NO: 199 or having at least 90% sequence identity, or at least 95% sequence identity, or at least 99% sequence identity to SEQ ID NO: 199.

In some embodiments, the self-replicating RNA molecule comprises a polynucleotide encoding an amino acid sequence of SEQ ID NO: 201 or having at least 90% sequence identity, or at least 95% sequence identity, or at least 99% sequence identity to SEQ ID NO: 201.

In some embodiments, the self-replicating RNA molecule comprises a polynucleotide encoding an amino acid sequence of SEQ ID NO: 203 or having at least 90% sequence identity, or at least 95% sequence identity, or at least 99% sequence identity to SEQ ID NO: 203.

In some embodiments, the self-replicating RNA molecule comprises a polynucleotide encoding an amino acid sequence of SEQ ID NO: 205 or having at least 90% sequence identity, or at least 95% sequence identity, or at least 99% sequence identity to SEQ ID NO: 205.

In some embodiments, the self-replicating RNA molecule comprises a polynucleotide encoding an amino acid sequence of SEQ ID NO: 207 or having at least 90% sequence identity, or at least 95% sequence identity, or at least 99% sequence identity to SEQ ID NO: 207.

In some embodiments, the self-replicating RNA molecule comprises a polynucleotide encoding an amino acid sequence of SEQ ID NO: 209 or having at least 90% sequence identity, or at least 95% sequence identity, or at least 99% sequence identity to SEQ ID NO: 209.

In some embodiments, the self-replicating RNA molecule comprises a polynucleotide encoding an amino acid sequence of SEQ ID NO: 211 or having at least 90% sequence identity, or at least 95% sequence identity, or at least 99% sequence identity to SEQ ID NO: 211.

In some embodiments, the self-replicating RNA molecule comprises a polynucleotide encoding an amino acid sequence of SEQ ID NO: 213 or having at least 90% sequence identity, or at least 95% sequence identity, or at least 99% sequence identity to SEQ ID NO: 213.

In some embodiments, the self-replicating RNA molecule comprises a polynucleotide encoding an amino acid sequence of SEQ ID NO: 215 or having at least 90% sequence identity, or at least 95% sequence identity, or at least 99% sequence identity to SEQ ID NO: 215.

In some embodiments, the self-replicating RNA molecule comprises a polynucleotide encoding an amino acid sequence of SEQ ID NO: 217 or having at least 90% sequence identity, or at least 95% sequence identity, or at least 99% sequence identity to SEQ ID NO: 217.

In some embodiments, the self-replicating RNA molecule comprises a polynucleotide encoding an amino acid sequence of SEQ ID NO: 219 or having at least 90% sequence identity, or at least 95% sequence identity, or at least 99% sequence identity to SEQ ID NO: 219.

In some embodiments, the self-replicating RNA molecule comprises a polynucleotide encoding an amino acid sequence of SEQ ID NO: 221 or having at least 90% sequence identity, or at least 95% sequence identity, or at least 99% sequence identity to SEQ ID NO: 221.

In some embodiments, the self-replicating RNA molecule comprises a polynucleotide encoding an amino acid sequence of SEQ ID NO: 223 or having at least 90% sequence identity, or at least 95% sequence identity, or at least 99% sequence identity to SEQ ID NO: 223.

In some embodiments, the self-replicating RNA molecule comprises a polynucleotide encoding an amino acid sequence of SEQ ID NO: 225 or having at least 90% sequence identity, or at least 95% sequence identity, or at least 99% sequence identity to SEQ ID NO: 225.

In some embodiments, the self-replicating RNA molecule comprises a polynucleotide encoding an amino acid sequence of SEQ ID NO: 227 or having at least 90% sequence identity, or at least 95% sequence identity, or at least 99% sequence identity to SEQ ID NO: 227.

In some embodiments, the self-replicating RNA molecule comprises a polynucleotide encoding an amino acid sequence of SEQ ID NO: 229 or having at least 90% sequence identity, or at least 95% sequence identity, or at least 99% sequence identity to SEQ ID NO: 229.

In some embodiments, the self-replicating RNA molecule comprises a polynucleotide encoding an amino acid sequence of SEQ ID NO: 231 or having at least 90% sequence identity, or at least 95% sequence identity, or at least 99% sequence identity to SEQ ID NO: 231.

In some embodiments, the self-replicating RNA molecule comprises a polynucleotide encoding an amino acid sequence of SEQ ID NO: 233 or having at least 90% sequence identity, or at least 95% sequence identity, or at least 99% sequence identity to SEQ ID NO: 233.

In some embodiments, the self-replicating RNA molecule comprises a polynucleotide encoding an amino acid sequence of SEQ ID NO: 235 or having at least 90% sequence identity, or at least 95% sequence identity, or at least 99% sequence identity to SEQ ID NO: 235.

In some embodiments, the self-replicating RNA molecule comprises a polynucleotide encoding an amino acid sequence of SEQ ID NO: 237 or having at least 90% sequence identity, or at least 95% sequence identity, or at least 99% sequence identity to SEQ ID NO: 237.

In some embodiments, the self-replicating RNA molecule comprises a polynucleotide encoding an amino acid sequence of SEQ ID NO: 239 or having at least 90% sequence identity, or at least 95% sequence identity, or at least 99% sequence identity to SEQ ID NO: 239.

In some embodiments, the self-replicating RNA molecule comprises a polynucleotide encoding an amino acid sequence of SEQ ID NO: 241 or having at least 90% sequence identity, or at least 95% sequence identity, or at least 99% sequence identity to SEQ ID NO: 241.

In some embodiments, the self-replicating RNA molecule comprises a polynucleotide encoding an amino acid sequence of SEQ ID NO: 243 or having at least 90% sequence identity, or at least 95% sequence identity, or at least 99% sequence identity to SEQ ID NO: 243.

In some embodiments, the self-replicating RNA molecule comprises a polynucleotide encoding an amino acid sequence of SEQ ID NO: 245 or having at least 90% sequence identity, or at least 95% sequence identity, or at least 99% sequence identity to SEQ ID NO: 245.

In some embodiments, the self-replicating RNA molecule comprises a polynucleotide encoding an amino acid sequence of SEQ ID NO: 247 or having at least 90% sequence identity, or at least 95% sequence identity, or at least 99% sequence identity to SEQ ID NO: 247.

In some embodiments, the self-replicating RNA molecule comprises a polynucleotide encoding an amino acid sequence of SEQ ID NO: 249 or having at least 90% sequence identity, or at least 95% sequence identity, or at least 99% sequence identity to SEQ ID NO: 249.

In some embodiments, the self-replicating RNA molecule comprises a polynucleotide encoding an amino acid sequence of SEQ ID NO: 251 or having at least 90% sequence identity, or at least 95% sequence identity, or at least 99% sequence identity to SEQ ID NO: 251.

In some embodiments, the self-replicating RNA molecule comprises a polynucleotide encoding an amino acid sequence of SEQ ID NO: 253 or having at least 90% sequence identity, or at least 95% sequence identity, or at least 99% sequence identity to SEQ ID NO: 253.

In some embodiments, the self-replicating RNA molecule comprises a polynucleotide encoding an amino acid sequence of SEQ ID NO: 255 or having at least 90% sequence identity, or at least 95% sequence identity, or at least 99% sequence identity to SEQ ID NO: 255.

In some embodiments, the self-replicating RNA molecule comprises a polynucleotide encoding an amino acid sequence of SEQ ID NO: 257 or having at least 90% sequence identity, or at least 95% sequence identity, or at least 99% sequence identity to SEQ ID NO: 257.

In some embodiments, the self-replicating RNA molecule comprises a polynucleotide encoding an amino acid sequence of SEQ ID NO: 259 or having at least 90% sequence identity, or at least 95% sequence identity, or at least 99% sequence identity to SEQ ID NO: 259.

In some embodiments, the self-replicating RNA molecule comprises a polynucleotide encoding an amino acid sequence of SEQ ID NO: 261 or having at least 90% sequence identity, or at least 95% sequence identity, or at least 99% sequence identity to SEQ ID NO: 261.

In some embodiments, the self-replicating RNA molecule comprises a polynucleotide encoding an amino acid sequence of SEQ ID NO: 263 or having at least 90% sequence identity, or at least 95% sequence identity, or at least 99% sequence identity to SEQ ID NO: 263.

In some embodiments, the self-replicating RNA molecule comprises a polynucleotide encoding an amino acid sequence of SEQ ID NO: 265 or having at least 90% sequence identity, or at least 95% sequence identity, or at least 99% sequence identity to SEQ ID NO: 265.

In some embodiments, the self-replicating RNA molecule comprises a polynucleotide encoding an amino acid sequence of SEQ ID NO: 267 or having at least 90% sequence identity, or at least 95% sequence identity, or at least 99% sequence identity to SEQ ID NO: 267.

In some embodiments, the self-replicating RNA molecule comprises a polynucleotide encoding an amino acid sequence of SEQ ID NO: 269 or having at least 90% sequence identity, or at least 95% sequence identity, or at least 99% sequence identity to SEQ ID NO: 269.

In some embodiments, the self-replicating RNA molecule comprises a polynucleotide encoding an amino acid sequence of SEQ ID NO: 271 or having at least 90% sequence identity, or at least 95% sequence identity, or at least 99% sequence identity to SEQ ID NO: 271.

In some embodiments, the self-replicating RNA molecule comprises a polynucleotide encoding an amino acid sequence of SEQ ID NO: 273 or having at least 90% sequence identity, or at least 95% sequence identity, or at least 99% sequence identity to SEQ ID NO: 273.

In some embodiments, the self-replicating RNA molecule comprises a polynucleotide encoding an amino acid sequence of SEQ ID NO: 275 or having at least 90% sequence identity, or at least 95% sequence identity, or at least 99% sequence identity to SEQ ID NO: 275.

In some embodiments, the self-replicating RNA molecule comprises a polynucleotide encoding an amino acid sequence of SEQ ID NO: 277 or having at least 90% sequence identity, or at least 95% sequence identity, or at least 99% sequence identity to SEQ ID NO: 277.

In some embodiments, the self-replicating RNA molecule comprises a polynucleotide encoding an amino acid sequence of SEQ ID NO: 279 or having at least 90% sequence identity, or at least 95% sequence identity, or at least 99% sequence identity to SEQ ID NO: 279.

In some embodiments, the self-replicating RNA molecule comprises a polynucleotide encoding an amino acid sequence of SEQ ID NO: 281 or having at least 90% sequence identity, or at least 95% sequence identity, or at least 99% sequence identity to SEQ ID NO: 281.

In some embodiments, the self-replicating RNA molecule comprises a polynucleotide encoding an amino acid sequence of SEQ ID NO: 283 or having at least 90% sequence identity, or at least 95% sequence identity, or at least 99% sequence identity to SEQ ID NO: 283.

In some embodiments, the self-replicating RNA molecule comprises a polynucleotide encoding an amino acid sequence of SEQ ID NO: 285 or having at least 90% sequence identity, or at least 95% sequence identity, or at least 99% sequence identity to SEQ ID NO: 285.

In some embodiments, the self-replicating RNA molecule comprises a polynucleotide encoding an amino acid sequence of SEQ ID NO: 287 or having at least 90% sequence identity, or at least 95% sequence identity, or at least 99% sequence identity to SEQ ID NO: 287.

In some embodiments, the self-replicating RNA molecule comprises a polynucleotide encoding an amino acid sequence of SEQ ID NO: 289 or having at least 90% sequence identity, or at least 95% sequence identity, or at least 99% sequence identity to SEQ ID NO: 289.

In some embodiments, the self-replicating RNA molecule comprises a polynucleotide encoding an amino acid sequence of SEQ ID NO: 291 or having at least 90% sequence identity, or at least 95% sequence identity, or at least 99% sequence identity to SEQ ID NO: 291.

In some embodiments, the self-replicating RNA molecule comprises a polynucleotide encoding an amino acid sequence of SEQ ID NO: 293 or having at least 90% sequence identity, or at least 95% sequence identity, or at least 99% sequence identity to SEQ ID NO: 293.

In some embodiments, the self-replicating RNA molecule comprises a polynucleotide encoding an amino acid sequence of SEQ ID NO: 295 or having at least 90% sequence identity, or at least 95% sequence identity, or at least 99% sequence identity to SEQ ID NO: 295.

In some embodiments, the self-replicating RNA molecule comprises a polynucleotide encoding an amino acid sequence of SEQ ID NO: 297 or having at least 90% sequence identity, or at least 95% sequence identity, or at least 99% sequence identity to SEQ ID NO: 297.

In some embodiments, the self-replicating RNA molecule comprises a polynucleotide encoding an amino acid sequence of SEQ ID NO: 299 or having at least 90% sequence identity, or at least 95% sequence identity, or at least 99% sequence identity to SEQ ID NO: 299.

In some embodiments, the self-replicating RNA molecule comprises a polynucleotide encoding an amino acid sequence of SEQ ID NO: 301 or having at least 90% sequence identity, or at least 95% sequence identity, or at least 99% sequence identity to SEQ ID NO: 301.

In some embodiments, the self-replicating RNA molecule comprises a polynucleotide encoding an amino acid sequence of SEQ ID NO: 303 or having at least 90% sequence identity, or at least 95% sequence identity, or at least 99% sequence identity to SEQ ID NO: 303.

In some embodiments, the self-replicating RNA molecule comprises a polynucleotide encoding an amino acid sequence of SEQ ID NO: 305 or having at least 90% sequence identity, or at least 95% sequence identity, or at least 99% sequence identity to SEQ ID NO: 305.

In some embodiments, the self-replicating RNA molecule comprises a polynucleotide encoding an amino acid sequence of SEQ ID NO: 307 or having at least 90% sequence identity, or at least 95% sequence identity, or at least 99% sequence identity to SEQ ID NO: 307.

In some embodiments, the self-replicating RNA molecule comprises a polynucleotide encoding an amino acid sequence of SEQ ID NO: 309 or having at least 90% sequence identity, or at least 95% sequence identity, or at least 99% sequence identity to SEQ ID NO: 309.

In some embodiments, the self-replicating RNA molecule comprises a polynucleotide encoding an amino acid sequence of SEQ ID NO: 311 or having at least 90% sequence identity, or at least 95% sequence identity, or at least 99% sequence identity to SEQ ID NO: 311.

In some embodiments, the self-replicating RNA molecule comprises a polynucleotide encoding an amino acid sequence of SEQ ID NO: 313 or having at least 90% sequence identity, or at least 95% sequence identity, or at least 99% sequence identity to SEQ ID NO: 313.

In some embodiments, the self-replicating RNA molecule comprises a polynucleotide encoding an amino acid sequence of SEQ ID NO: 315 or having at least 90% sequence identity, or at least 95% sequence identity, or at least 99% sequence identity to SEQ ID NO: 315.

In some embodiments, the self-replicating RNA molecule comprises a polynucleotide encoding an amino acid sequence of SEQ ID NO: 317 or having at least 90% sequence identity, or at least 95% sequence identity, or at least 99% sequence identity to SEQ ID NO: 317.

In some embodiments, the self-replicating RNA molecule comprises a polynucleotide encoding an amino acid sequence of SEQ ID NO: 319 or having at least 90% sequence identity, or at least 95% sequence identity, or at least 99% sequence identity to SEQ ID NO: 319.

In some embodiments, the self-replicating RNA molecule comprises a polynucleotide encoding an amino acid sequence of SEQ ID NO: 321 or having at least 90% sequence identity, or at least 95% sequence identity, or at least 99% sequence identity to SEQ ID NO: 321.

In some embodiments, the self-replicating RNA molecule comprises a polynucleotide encoding an amino acid sequence of SEQ ID NO: 323 or having at least 90% sequence identity, or at least 95% sequence identity, or at least 99% sequence identity to SEQ ID NO: 323.

In some embodiments, the self-replicating RNA molecule comprises a polynucleotide encoding an amino acid sequence of SEQ ID NO: 325 or having at least 90% sequence identity, or at least 95% sequence identity, or at least 99% sequence identity to SEQ ID NO: 325.

In some embodiments, the self-replicating RNA molecule comprises a polynucleotide encoding an amino acid sequence of SEQ ID NO: 327 or having at least 90% sequence identity, or at least 95% sequence identity, or at least 99% sequence identity to SEQ ID NO: 327.

In some embodiments, the self-replicating RNA molecule comprises a polynucleotide encoding an amino acid sequence of SEQ ID NO: 329 or having at least 90% sequence identity, or at least 95% sequence identity, or at least 99% sequence identity to SEQ ID NO: 329.

In some embodiments, the self-replicating RNA molecule comprises a polynucleotide encoding an amino acid sequence of SEQ ID NO: 331 or having at least 90% sequence identity, or at least 95% sequence identity, or at least 99% sequence identity to SEQ ID NO: 331.

In some embodiments, the self-replicating RNA molecule comprises a polynucleotide encoding an amino acid sequence of SEQ ID NO: 333 or having at least 90% sequence identity, or at least 95% sequence identity, or at least 99% sequence identity to SEQ ID NO: 333.

In some embodiments, the self-replicating RNA molecule comprises a polynucleotide encoding an amino acid sequence of SEQ ID NO: 335 or having at least 90% sequence identity, or at least 95% sequence identity, or at least 99% sequence identity to SEQ ID NO: 335.

In some embodiments, the self-replicating RNA molecule comprises a polynucleotide encoding an amino acid sequence of SEQ ID NO: 337 or having at least 90% sequence identity, or at least 95% sequence identity, or at least 99% sequence identity to SEQ ID NO: 337.

In some embodiments, the self-replicating RNA molecule comprises a polynucleotide encoding an amino acid sequence of SEQ ID NO: 339 or having at least 90% sequence identity, or at least 95% sequence identity, or at least 99% sequence identity to SEQ ID NO: 339.

In some embodiments, the self-replicating RNA molecule comprises a polynucleotide encoding an amino acid sequence of SEQ ID NO: 341 or having at least 90% sequence identity, or at least 95% sequence identity, or at least 99% sequence identity to SEQ ID NO: 341.

In some embodiments, the self-replicating RNA molecule comprises a polynucleotide encoding an amino acid sequence of SEQ ID NO: 343 or having at least 90% sequence identity, or at least 95% sequence identity, or at least 99% sequence identity to SEQ ID NO: 343.

In some embodiments, the self-replicating RNA molecule comprises a polynucleotide encoding an amino acid sequence of SEQ ID NO: 345 or having at least 90% sequence identity, or at least 95% sequence identity, or at least 99% sequence identity to SEQ ID NO: 345.

In some embodiments, the self-replicating RNA molecule comprises a polynucleotide encoding an amino acid sequence of SEQ ID NO: 347 or having at least 90% sequence identity, or at least 95% sequence identity, or at least 99% sequence identity to SEQ ID NO: 347.

In some embodiments, the self-replicating RNA molecule comprises a polynucleotide encoding an amino acid sequence of SEQ ID NO: 349 or having at least 90% sequence identity, or at least 95% sequence identity, or at least 99% sequence identity to SEQ ID NO: 349.

In some embodiments, the self-replicating RNA molecule comprises a polynucleotide encoding an amino acid sequence of SEQ ID NO: 351 or having at least 90% sequence identity, or at least 95% sequence identity, or at least 99% sequence identity to SEQ ID NO: 351.

In some embodiments, the self-replicating RNA molecule comprises a polynucleotide encoding an amino acid sequence of SEQ ID NO: 353 or having at least 90% sequence identity, or at least 95% sequence identity, or at least 99% sequence identity to SEQ ID NO: 353.

In some embodiments, the self-replicating RNA molecule comprises a polynucleotide encoding an amino acid sequence of SEQ ID NO: 355 or having at least 90% sequence identity, or at least 95% sequence identity, or at least 99% sequence identity to SEQ ID NO: 355.

In some embodiments, the self-replicating RNA molecule comprises a polynucleotide encoding an amino acid sequence of SEQ ID NO: 357 or having at least 90% sequence identity, or at least 95% sequence identity, or at least 99% sequence identity to SEQ ID NO: 357.

In some embodiments, the self-replicating RNA molecule comprises a polynucleotide encoding an amino acid sequence of SEQ ID NO: 359 or having at least 90% sequence identity, or at least 95% sequence identity, or at least 99% sequence identity to SEQ ID NO: 359.

In some embodiments, the self-replicating RNA molecule comprises a polynucleotide encoding an amino acid sequence of SEQ ID NO: 361 or having at least 90% sequence identity, or at least 95% sequence identity, or at least 99% sequence identity to SEQ ID NO: 361.

In some embodiments, the self-replicating RNA molecule comprises a polynucleotide encoding an amino acid sequence of SEQ ID NO: 363 or having at least 90% sequence identity, or at least 95% sequence identity, or at least 99% sequence identity to SEQ ID NO: 363.

In some embodiments, the self-replicating RNA molecule comprises a polynucleotide encoding an amino acid sequence of SEQ ID NO: 365 or having at least 90% sequence identity, or at least 95% sequence identity, or at least 99% sequence identity to SEQ ID NO: 365.

In some embodiments, the self-replicating RNA molecule comprises a polynucleotide encoding an amino acid sequence of SEQ ID NO: 367 or having at least 90% sequence identity, or at least 95% sequence identity, or at least 99% sequence identity to SEQ ID NO: 367.

In some embodiments, the self-replicating RNA molecule comprises a polynucleotide encoding an amino acid sequence of SEQ ID NO: 369 or having at least 90% sequence identity, or at least 95% sequence identity, or at least 99% sequence identity to SEQ ID NO: 369.

In some embodiments, the self-replicating RNA molecule comprises a polynucleotide encoding an amino acid sequence of SEQ ID NO: 371 or having at least 90% sequence identity, or at least 95% sequence identity, or at least 99% sequence identity to SEQ ID NO: 371.

In some embodiments, the self-replicating RNA molecule comprises a polynucleotide encoding an amino acid sequence of SEQ ID NO: 373 or having at least 90% sequence identity, or at least 95% sequence identity, or at least 99% sequence identity to SEQ ID NO: 373.

In some embodiments, the self-replicating RNA molecule comprises a polynucleotide encoding an amino acid sequence of SEQ ID NO: 375 or having at least 90% sequence identity, or at least 95% sequence identity, or at least 99% sequence identity to SEQ ID NO: 375.

In some embodiments, the self-replicating RNA molecule comprises a polynucleotide encoding an amino acid sequence of SEQ ID NO: 377 or having at least 90% sequence identity, or at least 95% sequence identity, or at least 99% sequence identity to SEQ ID NO: 377.

In some embodiments, the self-replicating RNA molecule comprises a polynucleotide encoding an amino acid sequence of SEQ ID NO: 379 or having at least 90% sequence identity, or at least 95% sequence identity, or at least 99% sequence identity to SEQ ID NO: 379.

In some embodiments, the self-replicating RNA molecule comprises a polynucleotide encoding an amino acid sequence of SEQ ID NO: 381 or having at least 90% sequence identity, or at least 95% sequence identity, or at least 99% sequence identity to SEQ ID NO: 381.

In some embodiments, the self-replicating RNA molecule comprises a polynucleotide encoding an amino acid sequence of SEQ ID NO: 383 or having at least 90% sequence identity, or at least 95% sequence identity, or at least 99% sequence identity to SEQ ID NO: 383.

In some embodiments, the self-replicating RNA molecule comprises a polynucleotide encoding an amino acid sequence of SEQ ID NO: 385 or having at least 90% sequence identity, or at least 95% sequence identity, or at least 99% sequence identity to SEQ ID NO: 385.

In some embodiments, the self-replicating RNA molecule comprises a polynucleotide encoding an amino acid sequence of SEQ ID NO: 387 or having at least 90% sequence identity, or at least 95% sequence identity, or at least 99% sequence identity to SEQ ID NO: 387.

In some embodiments, the self-replicating RNA molecule comprises a polynucleotide encoding an amino acid sequence of SEQ ID NO: 389 or having at least 90% sequence identity, or at least 95% sequence identity, or at least 99% sequence identity to SEQ ID NO: 389.

In some embodiments, the self-replicating RNA molecule comprises a polynucleotide encoding an amino acid sequence of SEQ ID NO: 391 or having at least 90% sequence identity, or at least 95% sequence identity, or at least 99% sequence identity to SEQ ID NO: 391.

In some embodiments, the self-replicating RNA molecule comprises a polynucleotide encoding an amino acid sequence of SEQ ID NO: 393 or having at least 90% sequence identity, or at least 95% sequence identity, or at least 99% sequence identity to SEQ ID NO: 393.

In some embodiments, the self-replicating RNA molecule comprises a polynucleotide encoding an amino acid sequence of SEQ ID NO: 395 or having at least 90% sequence identity, or at least 95% sequence identity, or at least 99% sequence identity to SEQ ID NO: 395.

In some embodiments, the self-replicating RNA molecule comprises a polynucleotide encoding an amino acid sequence of SEQ ID NO: 397 or having at least 90% sequence identity, or at least 95% sequence identity, or at least 99% sequence identity to SEQ ID NO: 397.

In some embodiments, the self-replicating RNA molecule comprises a polynucleotide encoding an amino acid sequence of SEQ ID NO: 399 or having at least 90% sequence identity, or at least 95% sequence identity, or at least 99% sequence identity to SEQ ID NO: 399.

In some embodiments, the self-replicating RNA molecule comprises a polynucleotide encoding an amino acid sequence of SEQ ID NO: 401 or having at least 90% sequence identity, or at least 95% sequence identity, or at least 99% sequence identity to SEQ ID NO: 401.

In some embodiments, the self-replicating RNA molecule comprises a polynucleotide encoding an amino acid sequence of SEQ ID NO: 403 or having at least 90% sequence identity, or at least 95% sequence identity, or at least 99% sequence identity to SEQ ID NO: 403.

In some embodiments, the self-replicating RNA molecule comprises a polynucleotide encoding an amino acid sequence of SEQ ID NO: 405 or having at least 90% sequence identity, or at least 95% sequence identity, or at least 99% sequence identity to SEQ ID NO: 405.

In some embodiments, the self-replicatin RNA molecule comprises a polynucleotide encoding an amino acid sequence of two or more of the polypeptides selected from SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145,147,149,151,157,161,163,165,171,173,179,185,187,197,199,207,213,217,221,223, 225,241,247,249,259,265,267,269,281,285,287,289,293,297,299,301,303,305,307,329, 331,333,335,337,339,341,343,345,349,353,355,357,359,361,363,365,367,369,371,375, 377, 379, 381, 383, 385, and 421, and fragments thereof.

Any of the above self-replicating RNA molecules can further comprise one or more of the following:
one or more nonstructural genes nsP1, nsP2, nsP3 and nsP4;
at least one of a DLP motif, a 5′ UTR, a 3′UTR and a Poly A; and
a subgenomic promoter.

In some embodiments, for example, the self-replicating RNA molecule can comprise one or more of the following:
one or more nonstructural genes nsP1, nsP2, nsP3 and nsP4;
at least one of a DLP motif, a 5′ UTR, a 3′UTR and a Poly A; and
a subgenomic promoter; and
an RNA encoding for any of the polypeptides of the disclosure, and operably linked to the subgenomic promoter.

In some embodiments, the self-replicating RNA molecule comprises an RNA sequence encoding a protein or peptide; 5′ and 3′ alphavirus untranslated regions; RNA sequences encoding amino acid sequences derived from New World alphavirus VEEV nonstructural proteins nsP1, nsP2, nsP3 and nsP4; a sub-genomic promoter that is operably linked to and regulates translation of the

RNA sequence encoding the protein; a 5′ cap and a 3′ poly-A tail; positive sense, single-stranded RNA; a DLP from Sindbis virus upstream of the non-structural protein 1(nsP1); a 2A ribosome skipping element; and a nspl nucleotide repeat downstream of the 5′-UTR and upstream of the DLP.

In some embodiments, the self-replicating RNA molecules may be at least 1 kb or at least 2 kb or at least 3 kb or at least 4 kb or at least 5 kb or at least 6 kb or at least 7 kb or at least 8 kb or at least 10 kb or at least 12 kb or at least 15 kb or at least 17 kb or at least 19 kb or at least 20 kb in size, or can be 100 bp-8 kb or 500 bp-8 kb or 500 bp-7 kb or 1-7 kb or 1-8 kb or 2-15 kb or 2-20 kb or 5-15 kb or 5-20 kb or 7-15 kb or 7-18 kb or 7-20 kb in size.

Any of the above-disclosed self-replicating RNA molecules can further include a coding sequence for an autoprotease peptide (e.g., autocatalytic self-cleaving peptide), where the coding sequence for the autoprotease is optionally operably linked upstream to the second nucleic acid sequence.

Generally, any proteolytic cleavage site known in the art can be incorporated into the nucleic acid molecules of the disclosure and can be, for example, proteolytic cleavage sequences that are cleaved post-production by a protease. Further suitable proteolytic cleavage sites also include proteolytic cleavage sequences that can be cleaved following addition of an external protease. As used herein the term “autoprotease” refers to a “self-cleaving” peptide that possesses autoproteolytic activity and is capable of cleaving itself from a larger polypeptide moiety. First identified in the foot-and-mouth disease virus (FMDV), a member of the picornavirus group, several autoproteases have been subsequently identified such as, for example, “2A like” peptides from equine rhinitis A virus (E2A), porcine teschovirus-1 (P2A) and Thosea asigna virus (T2A), and their activities in proteolytic cleavage have been shown in various ex vitro and in vivo eukaryotic systems. As such, the concept of autoproteases is available to one of skill in the art as many naturally occurring autoprotease systems have been identified. Well studied autoprotease systems are e.g. viral proteases, developmental proteins (e.g. HetR, Hedgehog proteins), RumA autoprotease domain, UmuD, etc.). Non-limiting examples of autoprotease peptides suitable for the compositions and methods of the present disclosure include the peptide sequences from porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof.

In some embodiments, the coding sequence for the autoprotease peptide is operably linked downstream of the DLP motif and upstream to the first and second polynucleotides.

In some embodiments, the autoprotease peptide comprises, or consists of, a peptide sequence selected from the group consisting of porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), and a combination thereof. In some embodiments, the autoprotease peptide includes a peptide sequence of porcine teschovirus-1 2A (P2A).

In some embodiments, the autoprotease peptide is porcine teschovirus-1 2A (P2A).

The incorporation of the P2A peptide in the modified viral RNA replicons of the present disclosure allows release of protein encoded by GOI (e.g. multiple myelomaneoantigen polypeptides of the disclosure) from the capsid-GOI fusion.

In some embodiments disclosed herein, the porcine teschovirus-1 2A (P2A) peptide sequence is engineered in-frame immediately after the DLP sequence and in-frame immediately upstream of all GOI.

Any of the above-disclosed self-replicating RNA molecules can further include a coding sequence downstream Loop (DLP) motif.

Some viruses have sequences capable of forming one or more stem-loop structures which regulate, for example increase, capsid gene expression. Viral capsid enhancer as used herein refers to a regulatory element comprising sequences capable of forming such stem-loop structures. In some examples, the stem-loop structures are formed by sequences within the coding sequence of a capsid protein and named Downstream Loop (DLP) sequence. As disclosed herein, these stem-loop structures or variants thereof can be used to regulate, for example increase, expression level of genes of interest. For example, these stem-loop structures or variants thereof can be used in a recombinant vector (e.g., in a heterologous viral genome) for enhancing transcription and/or translation of coding sequence operably linked downstream thereto.

Alphavirus replication in host cells is known to induce the double-stranded RNA-dependent protein kinase (PKR). PKR phosphorylates the eukaryotic translation initiation factor 2a (eIF2a). Phosphorylation of eIF2a blocks translation initiation of mRNA and in doing so keeps viruses from a completing a productive replication cycle. Members of the Alphavirus genus can resist the activation of antiviral RNA-activated protein kinase (PKR) by means of the dowsntream loop (DLP) present within the viral 26S transcripts, which allows an eIF2-independent translation initiation of these mRNAs. The DLP structure can stall a ribosome on the wild type AUG and this supports translation of the subgenomic mRNA without the requirement for functional eIF2a. The DLP structure was first characterized in Sindbis virus (SINV) 26S mRNA and also detected in Semliki Forest virus (SFV). Similar DLP structures have been reported to be present in at least 14 other members of the Alphavirus genus including New World (for example, MAYV, UNAV, EEEV (NA), EEEV (SA), AURAV) and Old World (SV, SFV, BEBV, RRV, SAG, GETV, MIDV, CHIKV, and ONNV) members. The DLP is located downstream from the AUG in SINV 26S mRNA and in other members of the Alphavirus genus.

In some embodiments, the nucleic acid molecules of the disclosure can include a coding sequence for a gene of interest (GOI) operably linked to DLP motif(s) and/or the coding sequence for the DLP motifs.

In some embodiments, the DLP of the self-replicating RNA molecule is derived from Sindbis virus.

In some embodiments, the downstream loop (DLP) comprises at least one RNA-stem-loop.

In some instances, DLP activity depends on the distance between the DLP motif and the initiation codon AUG (AUGi). The AUG-DLP spacing in Alphavirus 26S mRNAs is tuned to the topology of the ES6S region of the ribosomal 18S rRNA in a way that allows the placement of the AUGi in the P site of the 40S subunit stalled by the DLP, allowing the incorporation of Met-tRNA without the participation of eIF2. In the case of Sindbis virus, the DLP motif is found in the first \tilde{ }150 nt of the Sindbis subgenomic RNA. The hairpin is located downstream of the Sindbis capsid AUG initiation codon (AUG at nt 50 of the Sindbis subgenomic RNA) and results in stalling a ribosome such that the correct capsid gene AUG is used to initiate translation.

Without being bound by any particular theory, it is believed that placing the DLP motif upstream of a coding sequence for any GOI typically results in a fusion-protein of N-terminal capsid amino acids that are encoded in the hairpin region to the GOI encoded protein because initiation occurs on the capsid AUG not the GOI AUG.

In some embodiments, the self-replicating RNA molecule comprises a downstream loop placed upstream of the non-structural protein 1(nsP1).

In some embodiments, the downstream loop is placed upstream of the non-structural protein 1 (nsP1) and is joined to the nsP1 by a porcine teschovirus-1 2A (P2A) ribosome skipping element.

The DLP-containing self-replicating RNA of the disclosure can also be useful in conferring a resistance to the innate immune system in a subject. Unmodified RNA replicons are sensitive to the initial innate immune system state of cells they are introduced into. If the cells/individuals are in a highly active innate immune system state, the RNA replicon performance (e.g., replication and expression of a GOI) can be negatively impacted. By engineering a DLP to control initiation of protein translation, particularly of non-structural proteins, the impact of the pre-existing activation state of the innate immune system to influence efficient RNA replicon replication is removed or lessened. The result is more uniform and/or enhanced expression of a GOI that can impact vaccine efficacy or therapeutic impact of a treatment.

The DLP motif of the self-replicating RNA of the disclosure can confer efficient mRNA translation in cellular environments where cellular mRNA translation is inhibited. When a DLP is linked with translation of a replicon vector's non-structural protein genes the replicase and transcriptase proteins are capable of initiating functional replication in PKR activated cellular environments. When a DLP is linked with translation of subgenomic mRNAs robust GOI expression is possible even when cellular mRNA is restricted due to innate immune activation. Accordingly, engineering self-replicating RNA that contain DLP structures to help drive translation of both non-structural protein genes and subgenomic mRNAs provides a powerful way to overcome innate immune activation.

Examples of a self-replicating RNA vector comprising a DLP motif are described in US Patent Application Publication US2018/0171340 and the International Patent Application Publication WO2018106615, the content of which is incorporated herein by reference in its entirety.

Any of the above-disclosed self-replicating RNA molecules can further comprise nonstructural genes nsP1, nsP2, nsP3 and/or nsP4.

Alphavirus genomes encode non-structural proteins nsP1, nsP2, nsP3, and nsP4, which are produced as a single polyprotein precursor, sometimes designated P1234 (or nsP1-4 or nsP1234), and which is cleaved into the mature proteins through proteolytic. nsP1 can be about 60 kDa in size and may have methyltransferase activity and be involved in the viral capping reaction. nsP2 has a size of about 90 kDa and may have helicase and protease activity while nsP3 is about 60 kDa and contains three domains: a macrodomain, a central (or alphavirus unique) domain, and a hypervariable domain (HVD). nsP4 is about 70 kDa in size and contains the core RNA-dependent RNA polymerase (RdRp) catalytic domain. After infection the alphavirus genomic RNA is translated to yield a P1234 polyprotein, which is cleaved into the individual proteins.

Alphavirus genomes also encode three structural proteins: the core nucleocapsid protein C, and the envelope proteins P62, and E1 that associate as a heterodimer. Structural proteins are under the control of a subgenomic promoter and can be replaced by gene of interests (GIO).

In some embodiments, the self-replicating RNA molecule does not encode functional viral structural proteins.

In some embodiments of the present disclosure, the self-replicating RNA can lack (or not contain) the sequence(s) of at least one (or all) of the structural viral proteins (e.g. nucleocapsid protein C, and envelope proteins P62, 6K, and E1). In these embodiments, the sequences encoding one or more structural genes can be substituted with one or more sequences such as, for example, a coding sequence for at least one protein or peptide (or other gene of interest (GOI)) e.g. the multiple myeloma cancer neoantigen polypeptides of the disclosure.

In some embodiments, the self-replicating RNA lack sequences encoding alphavirus structural proteins; or do not encode alphavirus (or, optionally, any other) structural proteins. In some embodiments, the self-replicating RNA molecules are further devoided of a part or the entire coding region for one or more viral structural proteins. For example, the alphavirus expression system may be devoid of a portion of or the entire coding sequence for one or more of the viral capsid protein C, E1 glycoprotein, E2 glycoprotein, E3 protein and 6K protein.

In some embodiments, the self-replicating RNA molecule does not contain coding sequences for at least one of the structural viral proteins. In these instances, the sequences encoding structural genes can be substituted with one or more sequences such as, for example, a coding sequence for a multiple myeloma neoantigen polynucleotides of the disclosure.

The disclosure also provides a self-replicating RNA molecule comprising nonstructural genes nsP1, nsP2, nsP3 and nsP4, and wherein the self-replicating RNA molecule does not encode a functional viral structural protein.

In some embodiments, the self-replicating RNA molecule can include one or more nonstructural viral proteins. In certain embodiments, the one or more nonstructural viral proteins are derived from the same virus. In other embodiments, the one or more nonstructural proteins are derived from different viruses.

In some embodiments, the disclosure provides a self-replicating RNA molecule comprising the coding sequence for at least one, at least two, at least three, or at least four nonstructural viral proteins (e.g. nsP1, nsP2, nsP3, nsP4). The nsP1, nsP2, nsP3, and nsP4 proteins encoded by the replicon are functional or biologically active proteins.

In some embodiments, the self-replicating RNA molecule includes the coding sequence for a portion of the at least one nonstructural viral protein. For example, the self-replicating RNA molecules can include about 10%, 20%, 30%, 40%, 50%, 60%, 70%, 80%, 90%, 95%, 100%, or a range between any two of these values, of the encoding sequence for the at least one nonstructural viral protein. In some embodiments, the self-replicating RNA molecule can include the coding sequence for a substantial portion of the at least one nonstructural viral protein. As used herein, a “substantial portion” of a nucleic acid sequence encoding a nonstructural viral protein comprises enough of the nucleic acid sequence encoding the nonstructural viral protein to afford putative identification of that protein, either by manual evaluation of the sequence by one skilled in the art, or by computer-automated sequence comparison and identification using algorithms such as BLAST (see, for example, in “Basic Local Alignment Search Tool”; Altschul S F et al., J. Mol. Biol. 215:403-410, 1993).

In some embodiments, the self-replicating RNA molecule can include the entire coding sequence for the at least one nonstructural protein. In some embodiments, the self-replicating RNA molecule comprises substantially all the coding sequence for the native viral nonstructural proteins.

In some embodiments, the self-replicating RNA molecule comprises nsP1, nsP2, nsP3 and nsP4 sequences derived from the Venezuelan equine encephalitis virus (VEEV) and a DLP motif derived from the Sindbis virus (SIN).

In some embodiments, the self-replicating RNA molecules also have an RNA sub-sequence encoding an amino acid sequence derived from an alphavirus nsP3 macro domain, and an RNA sub-sequence encoding an amino acid sequence derived from an alphavirus nsP3 central domain. In various embodiments the macro and central domain(s) can both be derived from a New World wild type alphavirus nsP3 or can both be derived from an Old World wild type alphavirus nsP3 protein. In other embodiments, the macro domain can be derived from a New World wild type alphavirus macro domain and the central domain can be derived from an Old World wild type alphavirus central domain, or vice versa. The various domains can be of any sequence described herein. The self-replicating RNA molecules can also have an RNA sub-sequence encoding an amino acid sequence derived entirely from an Old World alphavirus nsP3 hypervariable domain; or can have an amino acid sequence having a portion derived from a New World alphavirus nsP3 hypervariable domain, and a portion derived from an Old World alphavirus nsP3 hypervariable domain. i.e. the hyper variable domain (HVD) can be a hybrid or chimeric New World/Old World sequence.

In some embodiments, the self-replicating RNA molecules can have an RNA sequence encoding amino acid sequences derived from a wild type New World alphavirus nsP1, nsP2, nsP3 and nsP4 protein sequences.

In some embodiments, the self-replicating RNA molecule contains non VEEV nonstructural proteins nsP1, nsP2, nsP3 and nsP4.

The accumulated experimental evidence has demonstrated that replication/amplification of VEEV and other alphavirus genomes and their defective interfering (DI) RNAs is determined by three promoter elements: (i) the conserved 3′-terminal sequence element (3′ CSE) and the following poly(A) tail; (ii) the 5′ UTR, which functions as a key promoter element for both negative- and positive-strand RNA synthesis; and (iii) the 51-nt conserved sequence element (51-nt CSE), which is located in the nsP1-coding sequence and functions as an enhancer of alphavirus genome replication (Kim et al., PNAS, 2014, 111: 10708-10713).

The 5′ and 3′ untranslated regions can be operably linked to any of the other sequences encoded by the replicon. The UTRs can be operably linked to a promoter and/or sequence encoding a protein or peptide by providing sequences and spacing necessary for recognition and transcription of the other encoded sequences.

Any of the above-disclosed self-replicating RNA molecules can further include an unmodified 5′ untranslated region (5′UTR).

In some embodiment, a self-replicating RNA molecule comprises a modified 5′ untranslated region (5′-UTR). For example, the modified 5′-UTR can comprise one or more nucleotide substitutions at position 1, 2, 4, or a combination thereof. Preferably, the modified 5′-UTR comprises a nucleotide substitution at position 2, more preferably, the modified 5′-UTR has a U->G substitution at position 2. Examples of such self-replicating RNA molecules are described in US Patent Application Publication US2018/0104359 and the International Patent Application Publication WO2018075235, the content of which is incorporated herein by reference in its entirety.

In some embodiments, the UTRs can be wild type New World or Old World alphavirus UTR sequences, or a sequence derived from any of them. The 5′ UTR can be of any suitable length, such as about 60 nt or 50-70 nt or 40-80 nt. In some embodiments the 5′ UTR can also have conserved primary or secondary structures (e.g. one or more stem-loop(s)) and can participate in the replication of alphavirus or of replicon RNA. The 3′ UTR can be up to several hundred nucleotides, for example it can be 50-900 or 100-900 or 50-800 or 100-700 or 200 nt-700 nt. The '3 UTR also can have secondary structures, e.g. a step loop, and can be followed by a polyadenylate tract or poly-A tail.

In some embodiments, the self-replicating RNA molecules can have a 3′ poly-A tail. It can also include a poly-A polymerase recognition sequence (e.g. AAUAAA) near its 3′ end.

In those instances where the self-replicating RNA molecule is to be packaged into a recombinant alphavirus particle, it can contain one or more sequences, so-called packaging signals, which serve to initiate interactions with alphavirus structural proteins that lead to particle formation. In some embodiments, the alphavirus particles comprise RNA derived from one or more alphaviruses; and structural proteins wherein at least one of said structural proteins is derived from two or more alphaviruses.

In some embodiments, the self-replicating RNA molecule comprises a VEEV derived vector wherein the structural viral proteins (e.g. nucleocapsid protein C, and envelope proteins P62, 6K, and E1) are removed and replaced by the coding sequence of the multiple myeloma neoantigen polynucleotides of the disclosure.

Previous studies have demonstrated that during VEEV and Sindbis virus infections only a small portion of viral nonstructural proteins (nsPs) is colocalized with dsRNA replication intermediates. Thus, it appears that a large fraction of nsPs are not involved in RNA replication (Gorchakov R, et al. (2008) A new role for ns polyprotein cleavage in Sindbis virus replication. J Virol 82(13):6218-6231). This has provided an opportunity to exploit the under used ns proteins for amplification of the subgenomic RNAs encoding proteins of interest, which is normally transcribed from the subgenomic promoter and is not further amplified.

In some embodiments, a fragment of the nsP1 of the self-replicating RNA molecule of the disclosure is duplicated downstream of the 5′-UTR and upstream of the DLP. In some embodiments the first 193 nucleotides of nsP1 are duplicated downstream of the 5′ UTR and upstream of the DLP

### Other Viral Vectors and Recombinant Viruses

The viral vector comprising the polynucleotide of the disclosure may be derived from other viral vectors including vectors derived from human adeno-associated viruses, such as AAV-2 (adeno-associated virus type 2). An attractive feature of AAV vectors is that they do not express any viral genes. The only viral DNA sequences included in the AAV vectors are the 145 bp inverted terminal repeats (ITR). Thus, as in immunization with naked DNA, the only gene expressed is that of the antigen, or antigen chimera. Additionally, AAV vectors are known to transduce both dividing and non-dividing cells, such as human peripheral blood monocyte-derived dendritic cells, with persistent transgene expression, and with the possibility of oral and intranasal delivery for generation of mucosal immunity. Moreover, the amount of DNA required appears to be much less by several orders of magnitude, with maximum responses at doses of 10¹⁰ to 10¹¹ particles or copies of DNA in contrast to naked DNA doses of 50 μg or about 10¹⁵ copies. AAV vectors are packaged by co-transfection of a suitable cell line (e.g., human 293 cells) with the DNA contained in the AAV ITR chimeric protein encoding constructs and an AAV helper plasmid ACG2 containing the AAV coding region (AAV rep and cap genes) without the ITRs. The cells are subsequently infected with the adenovirus Ad5. Vectors can be purified from cell lysates using methods known in the art (e.g., such as cesium chloride density gradient ultracentrifugation) and are validated to ensure that they are free of detectable replication-competent AAV or adenovirus (e.g., by a cytopathic effect bioassay).

Retroviral vectors may also be used. Retroviruses are a class of integrative viruses which replicate using a virus-encoded reverse transcriptase, to replicate the viral RNA genome into double stranded DNA which is integrated into chromosomal DNA of the infected cells (e.g., target cells). Such vectors include those derived from murine leukemia viruses, especially Moloney (Gilboa, et al., 1988, Adv. Exp. Med. Biol. 241: 29) or Friend's FB29 strains (Int. Pat. Publ. No. WO1995/01447). Generally, a retroviral vector is deleted of all or part of the viral genes gag, pol and env and retains 5′ and 3′ LTRs and an encapsidation sequence. These elements may be modified to increase expression level or stability of the retroviral vector. Such modifications include the replacement of the retroviral encapsidation sequence by one of a retrotransposon such as VL30 (see, e.g., U.S. Pat. No. 5,747,323). The polynucleotides of the disclosure may be inserted downstream of the encapsidation sequence, such as in opposite direction relative to the retroviral genome. Retroviral particles are prepared in the presence of a helper virus or in an appropriate complementation (packaging) cell line which contains integrated into its genome the retroviral genes for which the retroviral vector is defective (e.g. gag/pol and env). Such cell lines are described in the prior art (Miller and Rosman, 1989, BioTechniques 7: 980; Danos and Mulligan, 1988, Proc. Natl. Acad. Sci. USA 85: 6460; Markowitz, et al., 1988, Virol. 167: 400). The product of the env gene is responsible for the binding of the viral particle to the viral receptors present on the surface of the target cell and, therefore determines the host range of the retroviral particle. Packaging cell line, such as the PA317 cells (ATCC CRL 9078) or 293E16 (WO97/35996) containing an amphotropic envelope protein may therefore be used to allow infection of human and other species' target cells. The retroviral particles are recovered from the culture supernatant and may optionally be further purified according to standard techniques (e.g. chromatography, ultracentrifugation).

### Regulatory Elements

The polynucleotide or the heterologous polynucleotide of the disclosure may be operably linked to one or more regulatory elements in the vector. The regulatory elements may comprise promoters, enhancers, polyadenylation signals, repressors and the like. As used herein, the term “operably linked” is to be taken in its broadest reasonable context and refers to a linkage of polynucleotide elements in a functional relationship. A polynucleotide is “operably linked” when it is placed into a functional relationship with another polynucleotide. For instance, a promoter is operably linked to a coding sequence if it affects the transcription of the coding sequence.

Some of the commonly used enhancer and promoter sequences in expression vectors and viral vectors are, for example, human cytomegalovirus (hCMV), vaccinia P7.5 early/late promoter, CAG, SV40, mouse CMV (mCMV), EF-1 and hPGK promoters. Due to its high potency and moderate size of ca. 0.8 kB, the hCMV promoter is one of the most commonly used of these promoters. The hPGK promoter is characterized by a small size (ca. 0.4 kB), but it is less potent than the hCMV promoter. On the other hand, the CAG promoter consisting of a cytomegalovirus early enhancer element, promoter, first exon and intron of chicken beta-actin gene, and splice acceptor of the rabbit beta-globin gene, can direct very potent gene expression that is comparable to the hCMV promoter, but its large size makes it less suitable in viral vectors where space constraints can be a significant concern, e.g., in adenoviral vectors (AdV), adeno-associated viral vectors (AAV) or lentiviral vectors (LVs).

Additional promoters that may be used are Aotine Herpesvirus 1 major immediate early promoter (AoHV-1 promoter) described in Int. Pat. Publ. No. WO2018/146205. The promoter may be operably coupled to a repressor operator sequence, to which a repressor protein can bind in order to repress expression of the promoter in the presence of the repressor protein. In certain embodiments, the repressor operator sequence is a TetO sequence or a CuO sequence (see e.g. U.S. Pat. No. 9,790,256).

In certain cases, it may be desirable to express at least two separate polypeptides from the same vector. In this case each polynucleotide may be operably linked to the same or different promoter and/or enhancer sequences, or well-known bicistronic expression systems for example by utilizing internal ribosome entry site (IRES) from encephalomyocarditis virus may be used. Alternatively, bidirectional synthetic promoters may be used, such as a hCMV-rhCMV promoter and other promoters described in Int. Pat. Publ. No. WO2017/220499.

Polyadenylation signals may be derived from SV40 or bovine growth hormone (BGH).

The polynucleotide or the heterologous polynucleotide of the self-replicating RNA vectors of the disclosure may be operably linked to one or more regulatory elements in the vector. The self-replicating RNA vectors comprising the polynucleotide encoding the polypeptide of the disclosure can further comprise any regulatory elements to establish conventional function(s) of the vector, including but not limited to replication and expression of the polypeptide of the disclosure encoded by the polynucleotide sequence of the vector. Regulatory elements include, but are not limited to, a promoter, an enhancer, a polyadenylation signal, translation stop codon, a ribosome binding element, a transcription terminator, selection markers, origin of replication, etc. A vector can comprise one or more expression cassettes. An “expression cassette” is part of a vector that directs the cellular machinery to make RNA and protein. An expression cassette typically comprises three components: a promoter sequence, an open reading frame, and a 3′-untranslated region (UTR) optionally comprising a polyadenylation signal. An open reading frame (ORF) is a reading frame that contains a coding sequence of a protein of interest (e.g., the polypeptides of the disclosure) from a start codon to a stop codon. Regulatory elements of the expression cassette can be operably linked to a polynucleotide sequence encoding the polypeptides of interest. Any components suitable for use in an expression cassette described herein can be used in any combination and in any order to prepare vectors of the application.

The vector can comprise a promoter sequence, preferably within an expression cassette, to control expression of the polypeptides of the disclosure.

In a self-replicating RNA, the vector can further comprise additional polynucleotide sequences that stabilize the expressed transcript, enhance nuclear export of the RNA transcript, and/or improve transcriptional-translational coupling. Examples of such sequences include polyadenylation signals and enhancer sequences. A polyadenylation signal is typically located downstream of the coding sequence for a protein of interest (e.g., the polypeptides of the disclosure) within an expression cassette of the vector Enhancer sequences are regulatory DNA sequences that, when bound by transcription factors, enhance the transcription of an associated gene. An enhancer sequence is preferably located upstream of the polynucleotide sequence encoding the polypeptides of the disclosure, but downstream of a promoter sequence within an expression cassette of the vector.

Any enhancer sequence known to those skilled in the art in view of the present disclosure can be used.

Any of the components or sequences of the self-replicating RNA vector of the disclosure can be functionally or operably linked to any other of the components or sequences.

A promoter or UTR operably linked to a coding sequence is capable of effecting the transcription and expression of the coding sequence when the proper enzymes are present. The promoter need not be contiguous with the coding sequence, so long as it functions to direct the expression thereof. Thus, an operable linkage between an RNA sequence encoding a protein or peptide and a regulatory sequence (for example, a promoter or UTR) is a functional link that allows for expression of the polynucleotide of interest. Operably linked can also refer to sequences such as the sequences encoding the RdRp (e.g. nsP4), nsP1-4, the UTRs, promoters, and other sequences encoding in the RNA replicon, are linked so that they enable transcription and translation of the polypeptide and/or replication of the replicon. The UTRs can be operably linked by providing sequences and spacing necessary for recognition and translation by a ribosome of other encoded sequences.

A molecule is functional or biologically active if it performs at least 50% of the same activity as its natural (or wild type), corresponding molecule, but a functional molecule can also perform at least 60% or at least 70% or at least 90% or at least 95% or 100% of the same activity as its natural (or wild type) corresponding molecule. The self-replicating RNA molecules can also encode an amino acid sequence derived from or based on a wild type alphavirus amino acid sequence, meaning that they have at least 60% or at least 65% or at least 68% or at least 70% or at least 80% or at least 70% or at least 80% or at least 90% or at least 95% or at least 97% or at least 98% or at least 99% or 100% or 80-99% or 90-100% or 95-99% or 95-100% or 97-99% or 98-99% sequence identity with an amino acid sequence (which can be a corresponding sequence) encoded by a wild type RNA alphavirus genome, which can be a New World or Old World alphavirus genome. Sequences derived from other sequences can be up to 5% or up to 10% or up to 20% or up to 30% longer or shorter than the original sequence. In any of the embodiments the sequence identity can be at least 95% or at least 97% or at least 98% or at least 99% or 100% for any nucleotide sequence encoding (or amino acid sequence having) a G3BP or FXR binding site thereon. These sequences can also be up to 5% or up to 10% or up to 20% or up to 30% longer or shorter than the original sequence.

Cells of the Disclosure

The disclosure also provides a cell comprising or transduced with one or more vectors of the disclosure or one or more recombinant viruses of the disclosure.

Suitable cells include prokaryotic and eukaryotic cells, e.g., mammalian cells, yeast, fungi and bacteria (such as E. coli), such as Hek 293, CHO, PER.C6 or chicken embryonic fibroblast (CEF) cells. The cell can be used in vitro, such as for research or for production of the polypeptides or viruses, or the cell can be used in vivo. In some embodiments, the cell is a muscle cell. In some embodiments, the cell is an antigen presenting cell (APC). Suitable antigen presenting cells include dendritic cells, B lymphocytes, monocytes and macrophages.

The cells that are transfected with the polynucleotides or vectors of the disclosure may typically be obtained through cell culture repositories such as ATCC. APCs may be obtained from the peripheral blood using leukopheresis and “FICOLL/HYPAQUE” density gradient centrifugation (stepwise centrifugation through Ficoll and discontinuous Percoll density gradients). APCs may be isolated, cultured and engineered using known methods. For example, immature and mature dendritic cells may be generated from peripheral blood mononuclear cells (PBMCs) using known methods. In an exemplary method, isolated PBMCs are pre-treated to deplete T- and B-cells by means of an immunomagnetic technique. Lymphocyte-depleted PBMC are then cultured for in RPMI medium 9 e.g., about 7 days), supplemented with human plasma (preferably autologous plasma) and GM-CSF/IL-4, to generate dendritic cells. Dendritic cells are nonadherent when compared to their monocyte progenitors. Thus, on approximately day 7, non-adherent cells are harvested for further processing. The dendritic cells derived from PBMC in the presence of GM-CSF and IL-4 are immature, in that they can lose the nonadherence property and revert back to macrophage cell fate if the cytokine stimuli are removed from the culture. The dendritic cells in an immature state are effective in processing native protein antigens for the MHC class II restricted pathway (Romani, et al., J. Exp. Med. 169: 1169, 1989). Further maturation of cultured dendritic cells is accomplished by culturing for 3 days in a macrophage-conditioned medium (CM), which contains the necessary maturation factors. Mature dendritic cells are less able to capture new proteins for presentation but are much better at stimulating resting T cells (both CD4 and CD8) to grow and differentiate. Mature dendritic cells can be identified by their change in morphology, such as the formation of more motile cytoplasmic processes; by their nonadherence; by the presence of at least one of the following markers: CD83, CD68, HLA-DR or CD86; or by the loss of Fc receptors such as CD115 (reviewed in Steinman, Annu. Rev. Immunol. 9: 271, 1991). Mature dendritic cells can be collected and analyzed using typical cytofluorography and cell sorting techniques and devices, such as FACScan and FACStar. Primary antibodies used for flow cytometry are those specific to cell surface antigens of mature dendritic cells and are commercially available. Secondary antibodies can be biotinylated Igs followed by FITC- or PE-conjugated streptavidin. The vectors and recombinant viruses of the disclosure can be introduced into cells including APCs using the methods known in the art, including, but not limited to, transfection, electroporation, fusion, microinjection, viral-based delivery, or cell-based delivery.

### Vaccines and Pharmaceutical Compositions of the Disclosure

The disclosure also provides compositions comprising any of the polynucleotides, any of the polypeptides, and any of the vectors disclosed herein. In some embodiments, the compositions may comprise a vector comprising any of the nucleotides disclosed herein, wherein the vector is selected from Ad26, GAd20, MVA, or a self-replicating RNA molecule. In some embodiments, the compositions may comprise a recombinant virus or a self-replicating RNA molecule expressing any of the polypeptides or neoantigens disclosed herein. In some embodiments, the recombinant virus may be Ad26 virus, GAd20 virus or MVA virus.

Any of the compositions described above may comprise or may be formulated into a pharmaceutical composition comprising the composition and a pharmaceutically acceptable excipient.

The polypeptides or the heterologous polypeptides or fragments thereof, or the polynucleotides encoding them may be delivered into the subject utilizing any known delivery vehicle suitable for administering to the subject. It is expected that the polypeptides, the heterologous polypeptides or fragments thereof will be immunogenic in the subject regardless of the delivery vehicle used. The polynucleotide may be DNA or RNA, or derivatives thereof. RNA may be in the form of oligonucleotide RNA, tRNA (transfer RNA), snRNA (small nuclear RNA), rRNA (ribosomal RNA), mRNA (messenger RNA), antisense RNA, siRNA (small interfering RNA), self-replicating RNA, ribozymes, chimeric sequences, or derivatives of these groups.

The disclosure also provides a vaccine comprising the polynucleotide of the disclosure.

In some embodiments, the polynucleotide is DNA.

In some embodiments, the polynucleotides is RNA.

In some embodiments, RNA is mRNA.

The disclosure also provides a vaccine comprising the vector of the disclosure.

The disclosure also provides a vaccine comprising the rAd26 of the disclosure.

The disclosure also provides a vaccine comprising the rMVA of the disclosure.

The disclosure also provides a vaccine comprising the rGAd of the disclosure.

The disclosure also provides a vaccine comprising the rGAd20 of the disclosure.

The disclosure also provides a vaccine comprising the ChAd20 of the disclosure.

The disclosure also provides a vaccine comprising the self-replicating RNA molecule of the disclosure.

The disclosure also provides a vaccine comprising the cell of the disclosure.

The preparation of vaccine compositions is well known. Vaccines may comprise or may be formulated into a pharmaceutical composition comprising the vaccine and a pharmaceutically acceptable excipient.

“Pharmaceutically acceptable” refers to the excipient that at the dosages and concentrations employed, will not cause unwanted or harmful effects in the subjects to which they are administered and include carrier, buffers, stabilizers or other materials well known to those skilled in the art. The precise nature of the carrier or other material may depend on the route of administration, e.g., intramuscular, subcutaneous, oral, intravenous, cutaneous, intramucosal (e.g., gut), intranasal or intraperitoneal routes. Liquid carriers such as water, petroleum, animal or vegetable oils, mineral oil or synthetic oil may be included. Physiological saline solution, dextrose or other saccharide solution or glycols such as ethylene glycol, propylene glycol or polyethylene glycol may be included. Exemplary viral formulation are the Adenovirus World Standard (Hoganson et al, 2002): 20 mM Tris pH 8, 25 mM NaCl, 2.5% glycerol; or 20 mM Tris, 2 mM MgCl₂, 25 mM NaCl, sucrose 10% w/v, polysorbate-80 0.02% w/v; or 10-25 mM citrate buffer pH 5.9-6.2, 4-6% (w/w) hydroxypropyl-beta-cyclodextrin (HBCD), 70-100 mM NaCl, 0.018-0.035% (w/w) polysorbate-80, and optionally 0.3-0.45% (w/w) ethanol. Many other buffers can be used, and examples of suitable formulations for the storage and for pharmaceutical administration of purified pharmaceutical preparations are known.

### Adjuvants

The vaccine or pharmaceutical composition may comprise one or more adjuvants. Examples of such adjuvants include but are not limited to inorganic adjuvants (e.g. inorganic metal salts such as aluminium phosphate or aluminium hydroxide), organic adjuvants (e.g. saponins or squalene), oil-based adjuvants (e.g. Freund's complete adjuvant and Freund's incomplete adjuvant), liposomes, or biodegradable microspheres), virosomes, bacterial adjuvants (e.g. monophosphoryl lipid A, or muramyl peptides), synthetic adjuvants (e.g. non-ionic block copolymers, muramyl peptide analogues, or synthetic lipid A), or synthetic polynucleotides adjuvants (e.g polyarginine or polylysine). Suitable adjuvants include QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, Alum, and MF59. Other adjuvants that may be used include lectins, growth factors, cytokines and lymphokines such as alpha-interferon, gamma interferon, platelet derived growth factor (PDGF), granulocyte-colony stimulating factor (gCSF), granulocyte macrophage colony stimulating factor (gMCSF), tumor necrosis factor (TNF), epidermal growth factor (EGF), IL-1, IL-2, IL-4, IL-6, IL-8, IL-10, IL-12 or TLR agonists.

“Adjuvant” and “immune stimulant” are used interchangeably herein and are defined as one or more substances that cause stimulation of the immune system. In this context, an adjuvant is used to enhance an immune response to the vaccines or viral vectors described herein.

A pharmaceutical composition according to the disclosure may in certain embodiments be the vaccine of the disclosure.

Similarly, the polynucleotides, the heterologous polynucleotides, the polypeptides and the heterologous polypeptides of the disclosure may be formulated into pharmaceutical compositions comprising the polynucleotides, the heterologous polynucleotides, the polypeptides and the heterologous polypeptides and the pharmaceutically acceptable excipients.

In some embodiments, the pharmaceutical compositions are devoid of adjuvants.

### Nanoparticles

In some embodiments, the compositions of the disclosure may comprise nanoparticles. Any of the polypeptides or the heterologous polypeptides or fragments thereof, the polynucleotides encoding them or vectors comprising the polynucleotides of the disclosure may be attached to or in contact with nanoparticles for delivery to a subject. Delivery of the polypeptides or the heterologous polypeptides or fragments thereof, the polynucleotides encoding them or the vectors comprising the polynucleotides using nanoparticles may eliminate the need to include a virus or an adjuvant in the vaccine composition. The nanoparticles may contain immune danger signals that help to effectively induce an immune response to the peptides. The nanoparticles may induce dendritic cell (DC) activation and maturation, required for a robust immune response. The nanoparticles may contain non-self components that improve uptake of the nanoparticles and thus the peptides by cells, such as antigen presenting cells.

The nanoparticles are typically from about 1 nm to about 100 nm in diameter, such as about 20 nm to about 40 nm. Nanoparticles with a mean diameter of 20 to 40 nm may facilitate uptake of the nanoparticle to the cytosol (see. e.g. WO2019/135086). Exemplary nanoparticles are polymeric nanoparticles, inorganic nanoparticles, liposomes, lipid nanoparticles (LNP), an immune stimulating complex (ISCOM), a virus-like particle (VLP), or a self-assembling protein.

The nanoparticles may be calcium phosphate nanoparticles, silicon nanoparticles or gold nanoparticles. The polymeric nanoparticles may comprise one or more synthetic polymers, such as poly(d,l-lactide-co-glycolide) (PLG), poly(d,l-lactic-coglycolic acid) (PLGA), poly(g-glutamic acid) (g-PGA)m poly(ethylene glycol) (PEG), or polystyrene or one or more natural polymers such as a polysaccharide, for example pullulan, alginate, inulin, and chitosan. The use of a polymeric nanoparticles may be advantageous due to the properties of the polymers that may be include in the nanoparticle. For instance, the natural and synthetic polymers recited above may have good biocompatibility and biodegradability, a non-toxic nature and/or the ability to be manipulated into desired shapes and sizes. The polymeric nanoparticle may also form hydrogel nanoparticles, hydrophilic three-dimensional polymer networks with favorable properties including flexible mesh size, large surface area for multivalent conjugation, high water content, and high loading capacity for antigens. Polymers such as Poly (L-lactic acid) (PLA), PLGA, PEG, and polysaccharides are suitable for forming hydrogel nanoparticles. Inorganic nanoparticles typically have a rigid structure and comprise a shell in which an antigen is encapsulated or a core to which the antigen may be covalently attached. The core may comprise one or more atoms such as gold (Au), silver (Ag), copper (Cu) atoms, Au/Ag, Au/Cu, Au/Ag/Cu, Au/Pt, Au/Pd or Au/Ag/Cu/Pd or calcium phosphate (CaP).

In some embodiments, the nanoparticles may be liposomes. Liposomes are typically formed from biodegradable, non-toxic phospholipids and comprise a self-assembling phospholipid bilayer shell with an aqueous core. Liposomes may be an unilamellar vesicle comprising a single phospholipid bilayer, or a multilamellar vesicle that comprises several concentric phospholipid shells separated by layers of water. As a consequence, liposomes may be tailored to incorporate either hydrophilic molecules into the aqueous core or hydrophobic molecules within the phospholipid bilayers. Liposomes may encapsulate polynucleotides or the polypeptides or fragments thereof of the disclosure within the core for delivery. Liposomes and liposomal formulations can be prepared according to standard methods and are well known in the art, see, e.g., Remington's; Akimaru, 1995, Cytokines Mol. Ther. 1: 197-210; Alving, 1995, Immunol. Rev. 145: 5-31; Szoka, 1980, Ann. Rev. Biophys. Bioeng. 9: 467; U.S. Pat. Nos. 4,235,871; 4,501,728; and 4,837,028. The liposomes may comprise a targeting molecule for targeting liposome complexes to a particular cell type. Targeting molecule may comprise a binding partner (e.g., a ligand or receptor) for a biomolecule (e.g., a receptor or ligand) on the surface of a blood vessel or a cell found in a target tissue. Liposome charge is an important determinant in liposome clearance from the blood, with negatively charged liposomes being taken up more rapidly by the reticuloendothelial system (Juliano, 1975, Biochem. Biophys. Res. Commun. 63: 651) and thus having shorter half-lives in the bloodstream. Incorporating phosphatidylethanolamine derivatives enhances the circulation time by preventing liposomal aggregation. For example, incorporation of N-(omega-carboxy)acylamidophosphatidylethanolamines into large unilamellar vesicles of L-alpha-distearoylphosphatidylcholine dramatically increases the in vivo liposomal circulation lifetime (see, e.g., Ahl, 1997, Biochim. Biophys. Acta 1329: 370-382). Typically, liposomes are prepared with about 5 to 15 mole percent negatively charged phospholipids, such as phosphatidylglycerol, phosphatidylserine or phosphatidyl-inositol. Added negatively charged phospholipids, such as phosphatidylglycerol, also serve to prevent spontaneous liposome aggregation, and thus minimize the risk of undersized liposomal aggregate formation. Membrane-rigidifying agents, such as sphingomyelin or a saturated neutral phospholipid, at a concentration of at least about 50 mole percent, and 5 to 15 mole percent of monosialylganglioside can also impart desirably liposome properties, such as rigidity (see, e.g., U.S. Pat. No. 4,837,028). Additionally, the liposome suspension can include lipid-protective agents which protect lipids against free-radical and lipid-peroxidative damages on storage. Lipophilic free-radical quenchers, such as alpha-tocopherol and water-soluble iron-specific chelators, such as ferrioxianine, are preferred.

In some embodiments, the nanoparticles can include multilamellar vesicles of heterogeneous sizes. For example, vesicle-forming lipids can be dissolved in a suitable organic solvent or solvent system and dried under vacuum or an inert gas to form a thin lipid film. If desired, the film can be redissolved in a suitable solvent, such as tertiary butanol, and then lyophilized to form a more homogeneous lipid mixture which is in a more easily hydrated powder like form. This film is covered with an aqueous solution of the polypeptide or polynucleotide and allowed to hydrate, typically over a 15 to 60 minute period with agitation.

The size distribution of the resulting multilamellar vesicles can be shifted toward smaller sizes by hydrating the lipids under more vigorous agitation conditions or by adding solubilizing detergents such as deoxycholate. The hydration medium may comprise the nucleic acid at a concentration which is desired in the interior volume of the liposomes in the final liposome suspension. Suitable lipids that may be used to form multilamellar vesicles include DOTMA

DOGS or Transfectain™, DNERIE or DORIE, DC-CHOL, DOTAP™, Lipofectamine™ and glycerolipid compounds.

In some embodiments, the nanoparticle may be an immune-stimulating complex (ISCOM). ISCOMs are cage-like particles which are typically formed from colloidal saponin-containing micelles. ISCOMs may comprise cholesterol, phospholipid (such as phosphatidylethanolamine or phosphatidylcholine) and saponin (such as Quil A from the tree Quillaia saponaria).

In some embodiments, the nanoparticle may be a virus-like particle (VLP). VLPs are self-assembling nanoparticles that lack infectious nucleic acid, which are formed by self-assembly of biocompatible capsid protein. VLPs are typically about 20 to about 150 nm, such as about 20 to about 40 nm, about 30 to about 140 nm, about 40 to about 130 nm, about 50 to about 120 nm, about 60 to about 110 nm, about 70 to about 100 nm, or about 80 to about 90 nm in diameter. VLPs advantageously harness the power of evolved viral structure, which is naturally optimized for interaction with the immune system. The naturally-optimized nanoparticle size and repetitive structural order means that VLPs induce potent immune responses, even in the absence of adjuvant.

### Encapsulated Self-Replicating RNA Molecules

The self-replicating RNA molecules and/or compositions comprising the same can also be formulated as a nanoparticle using a combination of polymers, lipids, and/or other biodegradable agents, such as, but not limited to, calcium phosphate, polymers. Components can be combined in a core-shell, hybrid, and/or layer-by-layer architecture, to allow for fine-tuning of the nanoparticle so that delivery of the molecules and/or compositions of the disclosure can be enhanced.

The disclosed self-replicating RNA molecules and/or compositions comprising the self-replicating RNA molecules encoding any of the polypeptides of the disclosure can be encapsulated using one or more liposomes, lipoplexes, and/or lipid nanoparticles. Liposomes are artificially prepared vesicles which can primarily be composed of a lipid bilayer and can be used as a delivery vehicle for the administration of polynucleotides and self-replicating RNA molecules. Liposomes can be of different sizes such as, but not limited to, a multilamellar vesicle (MLV) which can be hundreds of nanometers in diameter and can contain a series of concentric bilayers separated by narrow aqueous compartments, a small unicellular vesicle (SUV) which can be smaller than 50 nm in diameter, and a large unilamellar vesicle (LUV) which can be between 50 and 500 nm in diameter. Liposome design can include, but is not limited to, opsonins or ligands in order to improve the attachment of liposomes to unhealthy tissue or to activate events such as, but not limited to, endocytosis. Liposomes can contain a low or a high pH in order to improve the delivery of the polynucleotides and self-replicating RNA molecules disclosed herein.

The formation of liposomes can depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In some embodiments, the self-replicating RNA molecule is encapsulated in, bound to or adsorbed on a liposome, a lipoplex, a lipid nanoparticle, or combinations thereof, preferably the self-replicating RNA molecule is encapsulated in a lipid nanoparticle.

In some embodiments, the self-replicating RNA molecule encoding the any of the polypeptides of the disclosure can be fully encapsulated within the lipid portion of the particle, thereby protecting the RNA from nuclease degradation. “Fully encapsulated” means that the RNA is not significantly degraded after exposure to serum or a nuclease assay that would significantly degrade free RNA. When fully encapsulated, preferably less than 25% of the nucleic acid in the particle is degraded in a treatment that would normally degrade 100% of free nucleic acid, more preferably less than 10%, and most preferably less than 5% of the nucleic acid in the particle is degraded. “Fully encapsulated” also means that the nucleic acid-lipid particles do not rapidly decompose into their component parts upon in vivo administration.

In some embodiments, the self-replicating RNA molecules and/or compositions of the disclosure comprising the same can be formulated in a lipid vesicle which can have crosslinks between functionalized lipid bilayers. In some embodiments, the self-replicating RNA molecules and/or compositions of the disclosure can be formulated in a lipid-polycation complex. The formation of the lipid-polycation complex can be accomplished by methods known in the art. As a non-limiting example, the polycation can include a cationic peptide or a polypeptide such as, but not limited to, polylysine, polyornithine and/or polyarginine and the cationic peptides. In some embodiments, the self-replicating RNA molecules and/or compositions disclosed herein can be formulated in a lipid-polycation complex which can further include a neutral lipid such as, but not limited to, cholesterol or dioleoyl phosphatidylethanolamine (DOPE). The lipid nanoparticle formulation can be influenced by, but not limited to, the selection of the cationic lipid component, the degree of cationic lipid saturation, the nature of the PEGylation, ratio of all components and biophysical parameters such as size.

In some embodiments, the self-replicating RNA molecule disclosed herein is encapsulated in a lipid nanoparticle (LNP). Lipid nanoparticles typically comprise four different lipids—an ionizable lipid, a neutral helper lipid, cholesterol, and a diffusible polyethylene glycol (PEG) lipid. LNPs are similar to liposomes but have slightly different function and composition. LNPs are designed toward encapsulating polynucleotides, such as DNA, mRNA, siRNA and sRNA. Traditional liposomes contain an aqueous core surrounded by one or more lipid bilayers. LNPs may assume a micelle-like structure, encapsulating polynucleotides in a non-aqueous core. LNPs typically contain a cationic lipid, a non-cationic lipid, and a lipid that prevents aggregation of the particle (e.g., a PEG-lipid conjugate). LNPs are useful for systemic applications, as they exhibit extended circulation lifetimes following intravenous (i.e.) injection and accumulate at distal sites (e.g., sites physically separated from the administration site). The LNPs may have a mean diameter of about 50 nm to about 150 nm, such as about 60 nm to about 130 nm, or about 70 nm to about 110 nm, or about 70 nm to about 90 nm, and are substantially nontoxic. Preparation of polynucleotide loaded LNPs are disclosed in, e.g., U.S. Pat. Nos. 5,976,567; 5,981,501; 6,534,484; 6,586,410; 6,815,432; and PCT Publication No. WO 96/40964. Polynucleotide containing LNPs are described for example in WO2019/191780.

In some embodiments, the lipid nanoparticles comprise a cationic lipid (e.g., one or more cationic lipids or salts thereof described herein), a phospholipid, and a conjugated lipid that inhibits aggregation of the particles (e.g., one or more PEG-lipid conjugates). The lipid particles can also include cholesterol. The lipid particles may encapsulate at least 1, 2, 3, 4, 5, 6, 7, 8, 9, 10, or more self-replicating RNA molecules that encode for one or more polypeptides.

In some embodiments, the LNP formulations comprising a polycationic composition can be used for the delivery of the self-replicating RNA molecules described herein in vivo and/or ex vitro. The disclosure further provides a LNP formulations comprising a cationic lipid.

The terms “cationic lipid” and “amino lipid” are used interchangeably herein to include those lipids and salts thereof having one, two, three, or more fatty acid or fatty alkyl chains and a pH-titratable amino head group (e.g., an alkylamino or dialkylamino head group). The cationic lipid is typically protonated (i.e., positively charged) at a pH below the pKa of the cationic lipid and is substantially neutral at a pH above the pKa. The cationic lipids may also be termed titratable cationic lipids. In some embodiments, the cationic lipids comprise: a protonatable tertiary amine (e.g., pH-titratable) head group; C18 alkyl chains, wherein each alkyl chain independently has 0 to 3 (e.g., 0, 1, 2, or 3) double bonds; and ether, ester, or ketal linkages between the head group and alkyl chains. Such cationic lipids include, but are not limited to, DSDMA, DODMA, DLinDMA, DLenDMA, γ-DLenDMA, DLin-K-DMA, DLin-K-C2-DMA (also known as DLin-C2K-DMA, XTC2, and C2K), DLin-K-C3-DM A, DLin-K-C4-DMA, DLen-C2K-DMA, y-DLen-C2K-DMA, DLin-M-C2-DMA (also known as MC2), DLin-M-C3-DMA (also known as MC3) and (DLin-MP-DMA)(also known as 1-B1 1).

The disclosure also provides an encapsulated self-replicating RNA molecule, wherein the cationic lipid comprises a protonatable tertiary amine. In some embodiments, the cationic lipid is di((Z)-non-2-en-1-yl) 8,8′-((((2-(dimethylamino)ethyl)thio)carbonyl)azanediyl) dioctanoate.

In some embodiments, the cationic lipid compounds are relatively non-cytotoxic. The cationic lipid compounds may be biocompatible and biodegradable. The cationic lipid may have a pKa in the range of approximately 5.5 to approximately 7.5, more preferably between approximately 6.0 and approximately 7.0.

The cationic lipid compounds described herein are particularly attractive for drug delivery for several reasons: they contain amino groups for interacting with DNA, RNA, other polynucleotides, and other negatively charged agents, for buffering the pH, for causing endo-osmolysis, for protecting the self-replicating RNA molecule to be delivered, they can be synthesized from commercially available starting materials; and/or they are pH responsive and can be engineered with a desired pKa.

Lipid nanoparticle formulations can be improved by replacing the cationic lipid with a biodegradable cationic lipid which is known as a rapidly eliminated lipid nanoparticle (reLNP). Ionizable cationic lipids, such as, but not limited to, DLinDMA, DLin-KC2-DMA, and DLin-MC3-DMA, have been shown to accumulate in plasma and tissues over time and can be a potential source of toxicity. The rapid metabolism of the rapidly eliminated lipids can improve the tolerability and therapeutic index of the lipid nanoparticles by an order of magnitude from a 1 mg/kg dose to a 10 mg/kg dose in rat. Inclusion of an enzymatically degraded ester linkage can improve the degradation and metabolism profile of the cationic component, while still maintaining the activity of the reLNP formulation. The ester linkage can be internally located within the lipid chain or it can be terminally located at the terminal end of the lipid chain. The internal ester linkage can replace any carbon in the lipid chain.

In some embodiments, the self-replicating RNA molecule can be packaged or encapsulated in cationic molecules, such as, polyamidoamine, dendritic polylysine, polyethylene irinine or polypropylene h-nine, polylysine, chitosan, DNA-gelatin coarcervates or DEAE dextran, dendrimers, or polyethylenimine (PEI).

In some embodiments, the lipid particles may comprise a lipid conjugate. The conjugated lipid is useful in that it prevents the aggregation of particles. Suitable conjugated lipids include, but are not limited to, PEG-lipid conjugates, cationic-polymer-lipid conjugates, and mixtures thereof.

PEG is a linear, water-soluble polymer of ethylene PEG repeating units with two terminal hydroxyl groups. PEGs are classified by their molecular weights; and include the following: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), monomethoxypolyethylene glycol-imida-zolyl-carbonyl (MePEG-IM), as well as such compounds containing a terminal hydroxyl group instead of a terminal methoxy group (e.g, HO-PEG-S, HO-PEG-S-NHS, HO-PEG-NH2).

The PEG moiety of the PEG-lipid conjugates described herein may comprise an average molecular weight ranging from 550 daltons to 10,000 daltons. Examples of PEG-lipids include, but are not limited to, PEG coupled to dialkyloxypropyls (PEG-DAA), PEG coupled to diacylglycerol (PEG-DAG), PEG coupled to phospholipids such as phosphatidylethanolamine (PEG-PE), PEG conjugated to ceramides, PEG conjugated to cholesterol or a derivative thereof, and mixtures thereof. In some embodiments, the PEG conjugated lipid is a DMG-PEG-2000.

The self-replicating RNA molecules can also be formulated in a particle comprising non-cationic lipids. Suitable non-cationic lipids include, for example, neutral uncharged, zwitterionic, or anionic lipids capable of producing a stable complex. Non-limiting examples of non-cationic lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoyl-phosphatidylcholine (POPC), palmitoylo-leoyl-phosphatidylethanolamine (POPE), palmitoyloleyol-phosphatidylglycerol (POPG), dioleoylphosphatidylethanolamine 4-(N-maleimidomethyl)-cyclohexane-1-carboxylate (DOPE-mal),phosphatidylethanolamine phosphatidylethanolamine phosphatidylethanolamine phosphatidylethanolamine, phosphatidylethanolamine, phosphatidylethanolaminedipalmitoyl-dimyristoyl-distearoyl-monomethyl-dimethyl-dielaidoyl-stearoyloleoyl-phosphatidylethanolamine (SOPE), lysophosphatidylcholine, dilinoleoylphosphatidylcholine, and mixtures thereof. Other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, palmitoyl, stearoyl, or oleoyl.

Additional examples of non-cationic lipids include sterols such as cholesterol and derivatives thereof. Non-limiting examples of cholesterol derivatives include polar analogues such as 5a-cholestanol, 5a-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5a-cholestanone, and cholesteryl decanoate; and mixtures thereof. In preferred embodiments, the cholesterol derivative is a polar analogue such as cholesteryl-(4′-hydroxy)-butyl ether. In some embodiments, the phospholipid is DSPC. In some embodiments, the non-cationic lipid present in lipid particles comprises or consists of a mixture of one or more phospholipids and cholesterol or a derivative thereof. In some embodiments where the lipid particles contain a mixture of phospholipid and cholesterol or a cholesterol derivative, the mixture may comprise up to 40 mol %, 45 mol %, 50 mol %, 55 mol %, or 60 mol % of the total lipid present in the particle.

In some embodiments, LNPs may comprise 30-70% cationic lipid compound, 0-60% cholesterol, 0-30% phospholipid, and 1-10% polyethylene glycol (PEG).

In some embodiments, the cationic lipid, zwitterion lipid, cholesterol and conjugated lipid are combined in a molar ratio of 50:7:40:3, respectively in the lipid nanoparticle

In some embodiments, the LNP formulations described herein can additionally comprise a permeability enhancer molecule.

In some embodiments, the nanoparticle formulations can be a carbohydrate nanoparticle comprising a carbohydrate carrier and self-replicating RNA molecule. As a non-limiting example, the carbohydrate carrier can include, but is not limited to, an anhydride-modified phytoglycogen or glycogen-type material, phtoglycogen octenyl succinate, phytoglycogen beta-dextrin, and anhydride-modified phytoglycogen beta-dextrin.

### Kits

The disclosure also provides a kit comprising one or more compositions, one or more polynucleotides, one or more polypeptides or one or more vectors of the disclosure. The disclosure also provides a kit comprising one or more recombinant viruses of the disclosure. The kits may be used to facilitate performing the methods described herein. In some embodiments, the kit further comprises reagents to facilitate entry of the vaccines of the disclosure into a cell, such as lipid-based formulations or viral packaging materials.

In some embodiments, the kit comprises one or more Ad26 vectors comprising any of the polynucleotides of the disclosure. In some embodiments, the kit comprises one or more MVA vectors comprising any of the polynucleotides of the disclosure. In some embodiments, the kit comprises one or more GAd20 vectors comprising any of the polynucleotides of the disclosure. In some embodiments, the kit comprises one or more self-replicating RNA molecules comprising any of the polynucleotides of the disclosure.

In some embodiments, the kit comprises an Ad26 vector of the disclosure and a MVA vector of the disclosure. In some embodiments, the kit comprises a GAd20 vector of the disclosure and a MVA vector of the disclosure. In some embodiments, the kit comprises an Ad26 vector of the disclosure and a Gad20 vector of the disclosure. In some embodiments, the kit comprises a self-replicating RNA molecule of the disclosure and a Gad20 vector of the disclosure. In some embodiments, the kit comprises a self-replicating RNA molecule of the disclosure and a MVA vector of the disclosure. In some embodiments, the kit comprises a self-replicating RNA molecule of the disclosure and an Ad26 vector of the disclosure. In some embodiments, the kit comprises one or more polynucleotides of the disclosure. In some embodiments, the kit comprises one or more polypeptides of the disclosure. In some embodiment, the kit comprises one or more cells of the disclosure.

In some embodiments, the kit comprises:

a first vaccine comprising a recombinant virus derived from Ad26, GAd20, or MVA, or a self-replicating RNA molecule comprising a heterologous polynucleotide encoding a heterologous polypeptide, wherein the heterologous polypeptide comprises two or more polypeptides selected from the group consisting SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, and 421, and fragments thereof; and

a second vaccine comprising a recombinant virus derived from Ad26, GAd20, or MVA, or a self-replicating RNA molecule comprising a heterologous polynucleotide encoding a heterologous polypeptide, wherein the heterologous polypeptide comprises two or more polypeptides selected from the group consisting SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, and 421, and fragments thereof.

In some embodiments, the kit comprises:

a first vaccine comprising a recombinant virus derived from Ad26, GAd20, or MVA or a self-replicating RNA molecule comprising a heterologous polynucleotide encoding a heterologous polypeptide, wherein the heterologous polypeptide comprises two or more polypeptides selected from the group consisting SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, and 421 and fragments thereof; and

a second vaccine comprising a recombinant virus derived from Ad26, Gad20 or MVA or a self-replicating RNA molecule comprising a heterologous polynucleotide encoding a heterologous polypeptide, wherein the heterologous polypeptide comprises two or more polypeptides selected from the group consisting SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, and 421, and fragments thereof.

### Other Molecules

### Multiple Myeloma Neoantigen/HLA Complexes

The disclosure also provides a protein complex comprising a multiple myeloma neoantigen and HLA. The disclosure also provides a protein complex comprising a fragment of the multiple myeloma neoantigen and HLA. The disclosure also provides a protein complex comprising a variant of the multiple myeloma neoantigen and HLA. The disclosure also provides a protein complex comprising a variant of a fragment of the multiple myeloma neoantigen and HLA.

In some embodiments, the multiple myeloma neoantigen comprises the polypeptide sequence selected from the group of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405 407, and 421, and fragments thereof.

In some embodiments, HLA is class I HLA. In some embodiments, HLA is class II HLA. In some embodiments, HLA is HLA-A. In some embodiments, HLA is HLA-B. In some embodiments, HLA is HLA-C. In some embodiments, HLA is HLA-DP. In some embodiments, HLA is HLA-DQ. In some embodiments, HLA is HLA-DR. In some embodiments, HLA is HLA-A*01:01, A*02:01, A*03:01, A*24:02, B*07:02 or B*08:01.

The complex of the multiple myeloma neoantigen and HLA may be used to for example isolate cognate T cells in vitro or in vivo. The complex of the multiple myeloma neoantigen and HLA may also be conjugated to a detectable label and used as a detection agent to detect, visualize or isolate cognate TCR or T cells expressing the cognate TCR. The complex of the multiple myeloma neoantigen and HLA may also be conjugated to a cytotoxic agent and used to deplete or reduce the number of cells expressing the cognate TCR. The complex may be in its native configuration or alternatively the multiple myeloma neoantigen and/or the HLA may be engineered. In some embodiments, the protein complex of the disclosure is conjugated to a detection agent or a cytotoxic agent.

Engineering concepts include covalent coupling of the peptide to the HLA, for example by using covalent linkers that may be cleavable. The multiple myeloma neoantigen and HLA complex may be a monomer or a multimer. The multiple myeloma neoantigen and HLA complex may be coupled to a toxin or a detection agent. Various engineering concepts include expressing the complex as a covalent multiple myeloma neoantigen-β2-α2-α1-β1 chain or multiple myeloma neoantigen-α chain, e.g. as a soluble complex. Linkers which are at least 15 amino acids long may be used between the multiple myeloma neoantigen and the HLA. Alternatively, the complex may be expressed as covalently coupled multiple myeloma neoantigen-single chain β1-α1. The multiple myeloma neoantigen/HLA complex may also be expressed as a full length HLAαβ chains to which the multiple myeloma neoantigen is covalently coupled to the N-terminus of the α chain or alternatively the multiple myeloma neoantigen is associated with the αβ chain via non-covalent interactions. Various expression formats are disclosed in U.S. Pat. Nos. 5,976,551, 5,734,023, 5,820,866, 7,141,656B2, 6,270,772B1 and 7,074,905B2. Additionally, the HLA may be expressed as a single chain construct which is mutated at al chain or stabilized via disulfide bonds via α2 and β2 domains as described in U.S. Pat. Nos. 8,377,447B2 and 8,828,379B2. The multiple myeloma neoantigen or fragment thereof may be coupled to the HLA via light sensitive or periodate sensitive cleavable linkers as described in U.S. Pat. Nos. 9,079,941B2. The multiple myeloma neoantigen/HLA complexes may be engineered into multimeric format. Multimeric formats may be generated by incorporating a reactive side chain to the C-terminus of the HLA α or β chain to facilitate cross-linking of two or more multiple myeloma neoantigen/HLA complexes, as described in U.S. Pat. Nos. 7,074,904B2. Alternatively, a biotinylation recognition sequence BirA may be incorporated to the C-terminus of the HLA α or β chain which is subsequently biotinylated and the multimer is formed by binding to avidin/streptavidin as described in U.S. Pat. No. 563,536. Multimeric multiple myeloma neoantigen/HLA complexes may further be generated utilizing Fc fusions, coupling the multiple myeloma neoantigen/HLA complexes in dextran carriers, oligomerizing the via coiled-coil domains, utilizing additional biotinylation peptides or conjugating the multiple myeloma neoantigen/HLA complexes onto nanoparticles or chelate carrier as is described in U.S. Pat. Nos. 6,197,302B1, 6,268,411B1, US20150329617A1, EP1670823B1, EP1882700B1, EP2061807B1, US20120093934A1, US20130289253A1, US20170095544A1, US20170003288A1 and WO2017015064A1.

The disclosure provides protein complex comprising human leucocyte antigen (HLA) and a polypeptide of the disclosure comprising the amino acid sequence of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, or 421, or a fragment thereof.

In some embodiments, the HLA may comprise class I or class II.

In some embodiments, the HLA may comprise HLA-A, HLA-B or HLA-C.

In some embodiments, the HLA may comprise HLA-DP, HLA-DQ or HLA-DR.

In some embodiments, the HLA may comprise class I alleles HLA-A*01:01, A*02:01, A*03:01, A*24:02, B*07:02 or B*08:01.

### Proteinaceous Molecules

The disclosure also provides an isolated proteinaceous molecule that specifically binds the polypeptide of the disclosure or the complex of the HLA and the polypeptide.

In some embodiments, the proteinaceous molecule is an antibody, an alternative scaffold, a chimeric antigen receptor (CAR) or a T cell receptor (TCR).

In some embodiments, the disclosure also provides proteinaceous molecules that bind the polypeptide of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217,219,221,223,225,227,229,231,233,235,237,239,241,243,245,247,249,251,253,255, 257,259,261,263,265,267,269,271,273,275,277,279,281,283,285,287,289,291,293,295, 297,299,301,303,305,307,309,311,313,315,317,319,321,323,325,327,329,331,333,335, 337,339,341,343,345,347,349,351,353,355,357,359,361,363,365,367,369,371,373,375, 377,379,381,383,385,387,389,391,393,395,397,399,401,403,405,407,or421. The proteinaceous molecules have insubstantial binding to a wild-type protein the neoantigen is a variant of.

The disclosure also provides proteinaceous molecules that bind a multiple myeloma neoantigen/HLA complex, wherein the multiple myeloma neoantigen comprises a polypeptide of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, or 421, or fragments thereof.

In some embodiments, the proteinaceous molecule is an antigen binding fragment of an antibody.

In some embodiments, the proteinaceous molecule is a multispecific molecule. In some embodiments, the proteinaceous molecule is a bispecific molecule. In some embodiments, the proteinaceous molecule is a trispecific molecule. In some embodiments, the multispecific molecule binds two or more distinct multiple myeloma neoantigens. In some embodiments, the multispecific molecule binds a multiple myeloma neoantigen and a T cell receptor (TCR) complex. In some embodiments, the multispecific molecule binds two or more distinct multiple myeloma neoantigens and a T cell receptor (TCR) complex.

In some embodiments, the proteinaceous molecule is an antibody.

In some embodiments, the proteinaceous molecule is a multispecific antibody. In some embodiments, the proteinaceous molecule is a bispecific antibody. In some embodiments, the proteinaceous molecule is a trispecific antibody. In some embodiments, the proteinaceous molecule is a T cell redirecting molecule.

In instances where the multiple myeloma neoantigen of the disclosure is part of an extracellular domain of a protein, the multiple myeloma neoantigen may be used as a tumor associated antigen for recruiting T cells to tumors or targeting CAR-T and other cellular therapies to tumor utilizing antigen binding domains that selectively bind the multiple myeloma neoantigen on tumor cells.

In instances in which the multiple myeloma neoantigen is part of an intracellular domain, antigen binding domains having the ability to be delivered into intracellular compartments conjugated to cytotoxic agent or a therapeutic agent may be used as therapeutics. Alternatively, cells engineered to express cognate TCR which bind the multiple myeloma neoantigen/HLA complex may be used as therapeutics.

In some embodiments, the proteinaceous molecule is an alternative scaffold.

In some embodiments, the proteinaceous molecule is a chimeric antigen receptor (CAR).

In some embodiments, the proteinaceous molecule is a T cell receptor (TCR).

Binding of the proteinaceous molecule to the multiple myeloma neoantigen or the multiple myeloma neoantigen/HLA complex of the disclosure may be determined experimentally using any suitable method. Such methods may utilize ProteOn XPR36, Biacore 3000 or KinExA instrumentation, ELISA or competitive binding assays known to those skilled in the art. The measured binding may vary if measured under different conditions (e.g., osmolarity, pH). Thus, measurements of affinity and other binding parameters (e.g., K_{D}, Kₒₙ, K_{off}) are typically made with standardized conditions and a standardized buffer, such as the buffer described herein. Skilled in the art will appreciate that the internal error for affinity measurements for example using Biacore 3000 or ProteOn (measured as standard deviation, SD) may typically be within 5-33% for measurements within the typical limits of detection. “Insubstantial” refers to binding that is 100-fold less when compared to the measured binding of the proteinaceous molecule to the multiple myeloma neoantigen of the disclosure. The proteinaceous molecule of the disclosure may further be characterized for their activity and function using know methods and those described herein, such as ability of the proteinaceous molecules to kill cells expressing the multiple myeloma neoantigens or multiple myeloma neoantigen/HLA complexes.

### Antibodies and Antigen Binding Domains

Antibodies and antigen binding domains that specifically bind the multiple myeloma neoantigens or the multiple myeloma neoantigen/HLA complexes may be generated using known methods. Such antibodies may include immunoglobulin molecules of any type (e.g., IgG, IgE, IgM, IgD, IgA and IgY), class (e.g., IgG1, IgG2, IgG3, IgG4, IgA1, and IgA2) or subclass.

For example, the hybridoma method of Kohler and Milstein, Nature 256:495, 1975 may be used to generate monoclonal antibodies. In the hybridoma method, a mouse or other host animal, such as a hamster, rat or monkey, is immunized with one or more multiple myeloma neoantigens, or/multiple myeloma neoantigen/HLA complexes followed by fusion of spleen cells from immunized animals with myeloma cells using standard methods to form hybridoma cells (Goding, Monoclonal Antibodies: Principles and Practice, pp.59-103 (Academic Press, 1986)). Colonies arising from single immortalized hybridoma cells are screened for production of antibodies with desired properties, such as specificity of binding and affinity for the multiple myeloma neoantigen of the disclosure.

Various host animals may be used to produce the antibodies. For example, Balb/c mice, rats or chickens may be used to generate antibodies containing the VH/VL pair, and llama and alpaca may be used to generated heavy chain only (WH) antibodies using standard immunization protocols. The antibodies made in non-human animals may be humanized using various technologies to generate more human-like sequences.

Exemplary humanization techniques including selection of human acceptor frameworks are known and include CDR grafting (U.S. Pat. No. 5,225,539), SDR grafting (U.S. Pat. No. 6,818,749), resurfacing (Padlan, (1991) Mol Immunol 28:489-499), Specificity Determining Residues Resurfacing (U.S. Patent Publ. No. 2010/0261620), human framework adaptation (U.S. Pat. No. 8,748,356) and superhumanization (U.S. Pat. No. 7,709, 226). In these methods, CDRs of parental antibodies are transferred onto human frameworks that may be selected based on their overall homology to the parental frameworks, based on similarity in CDR length, or canonical structure identity, or any combination thereof.

Humanized antibodies may be further optimized to improve their selectivity or affinity to a desired antigen by incorporating altered framework support residues to preserve binding affinity (backmutations) by techniques such as those described in Int. Patent Publ. Nos. WO1090/007861 and WO1992/22653, or by introducing variation at any of the CDRs for example to improve affinity of the antibody.

Transgenic animals, such as mice or rats carrying human immunoglobulin (Ig) loci in their genome may be used to generate human antibodies against the multiple myeloma neoantigens of the multiple myeloma neoantigen/HLA complexes, and are described in for example U.S. Pat. No. 6,150,584, Int. Patent Publ. No. WO99/45962, Int. Patent Publ. Nos. WO2002/066630, WO2002/43478, WO2002/043478 and WO1990/04036, Lonberg et al (1994) Nature 368:856-9; Green et al (1994) Nature Genet. 7:13-21; Green & Jakobovits (1998) Exp. Med. 188:483-95; Lonberg and Huszar (1995) Int Rev Immunol 13:65-93; Bruggemann et al., (1991) Eur J Immunol 21:1323-1326; Fishwild et al., (1996) Nat Biotechnol 14:845-851; Mendez et al., (1997) Nat Genet 15:146-156; Green (1999) J Immunol Methods 231:11-23; Yang et al., (1999) Cancer Res 59:1236-1243; Brüggemann and Taussig (1997) Curr Opin Biotechnol 8:455-458. The endogenous immunoglobulin loci in such animal may be disrupted or deleted, and at least one complete or partial human immunoglobulin locus may be inserted into the genome of the animal using homologous or non-homologous recombination, using transchromosomes, or using minigenes. Companies such as Regeneron (http://_www_regeneron_com), Harbour Antibodies (http://_www_harbourantibodies_com), Open Monoclonal Technology, Inc. (OMT) (http://_www_omtinc_net), KyMab (http://www_kymab_com), Trianni (http://_www.trianni_com) and Ablexis (http://_www_ablexis_com) may be engaged to provide human antibodies directed against a selected antigen using technologies as described above.

Human antibodies may be selected from a phage display library, where the phage is engineered to express human immunoglobulins or portions thereof such as Fabs, single chain antibodies (scFv), domain antibodies or unpaired or paired antibody variable regions (Knappik et al., (2000) J Mol Biol 296:57-86; Krebs et al., (2001) J Immunol Meth 254:67-84; Vaughan et al., (1996) Nature Biotechnology 14:309-314; Sheets et al., (1998) PITAS (USA) 95:6157-6162; Hoogenboom and Winter (1991) J Mol Biol 227:381; Marks et al., (1991) J Mol Biol 222:581). The antibodies of the disclosure may be isolated for example from phage display library expressing antibody heavy and light chain variable regions as heterologous polypeptides with bacteriophage pIX coat protein as described in Shi et al., (2010) J Mol Biol 397:385-96, and Int. Patent Publ. No. WO09/085462). The libraries may be screened for phage binding to the multiple myeloma neoantigen or the multiple myeloma neoantigen/HLA complex and the obtained positive clones may be further characterized, the Fabs isolated from the clone lysates, and expressed as full length IgGs. Such phage display methods for isolating human antibodies are described in for example: U.S. Pat. Nos. 5,223,409, 5,403,484, 5,571,698, 5,427,908, 5, 580,717, 5,969,108, 6,172,197, 5,885,793; 6,521,404; 6,544,731; 6,555,313; 6,582,915 and 6,593,081. The antibodies may further be tested for their binding to the HLA/neoantigen complex or to the neoantigen alone.

Preparation of immunogenic antigens and monoclonal antibody production may be performed using any suitable technique, such as recombinant protein production or by chemical synthesis of peptides. The immunogenic antigens may be administered to an animal in the form of purified protein, or protein mixtures including whole cells or cell or tissue extracts, or the antigen may be formed de novo in the animal's body from nucleic acids encoding said antigen or a portion thereof.

Antigen binding domains that specifically bind the multiple myeloma neoantigen or multiple myeloma neoantigen/HLA complexes may also be derived from the antibodies described herein. Antigen binding domains include single chain antibodies, Fab fragments, Fv fragments, single-chain Fv fragments (scFv), VHH domains, VH, VL, alternative scaffolds (e.g. non-antibody antigen binding domains), a divalent antibody fragment such as an (Fab)2′-fragment, F(ab′) fragments, disulfide-linked Fvs (sdFv), intrabodies, minibodies, diabodies, triabodies and decabodies.

Bispecific and multispecific antibodies that specifically bind the multiple myeloma neoantigen or multiple myeloma neoantigen/HLA complexes and a second antigen may be generated using known methods. The second antigen may be a T cell receptor complex (TCR complex). The second antigen may be CD3 within the TCR complex. The bispecific and multispecific antibodies that specifically bind the multiple myeloma neoantigen or multiple myeloma neoantigen/HLA complexes of the disclosure and the second antigen may be engineered into any multivalent format using any known antigen binding domains format that specifically bind the multiple myeloma neoantigens or multiple myeloma neoantigen/HLA complexes and the second antigen. The antigen binding domain that specifically bind the multiple myeloma neoantigen or multiple myeloma neoantigen/HLA complex may be conjugated to one or more Fc domains or fragment thereof, or optionally to other scaffolds such as half-life extending moieties including albumin, PEG or transferrin.

Multispecific antibodies that specifically bind two or more multiple myeloma neoantigens may provide a benefit in terms of improved specificity in targeting tumor cells expressing the multiple myeloma neoantigens.

The antigen binding domains that specifically bind the multiple myeloma neoantigen or multiple myeloma neoantigen/HLA complexes may be engineered into full length multispecific antibodies which are generated using Fab arm exchange, in which substitutions are introduced into two monospecific bivalent antibodies within the Ig constant region CH3 domain which promote Fab arm exchange in vitro. In the methods, two monospecific bivalent antibodies are engineered to have certain substitutions at the CH3 domain that promote heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol, preferably a reducing agent selected from the group consisting of: 2-mercaptoethylamine, dithiothreitol and tris(2-carboxyethyl)phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

CH3 mutations that may be used include technologies such as Knob-in-Hole mutations (Genentech), electrostatically-matched mutations (Chugai, Amgen, NovoNordisk, Oncomed), the Strand Exchange Engineered Domain body (SEEDbody) (EMD Serono), Duobody® mutations (Genmab), and other asymmetric mutations (e.g. Zymeworks).

Knob-in-hole mutations are disclosed for example in WO1996/027011 and include mutations on the interface of CH3 region in which an amino acid with a small side chain (hole) is introduced into the first CH3 region and an amino acid with a large side chain (knob) is introduced into the second CH3 region, resulting in preferential interaction between the first CH3 region and the second CH3 region. Exemplary CH3 region mutations forming a knob and a hole are T366Y/F405A, T366W/F405W, F405W/Y407A, T394W/Y407T, T394S/Y407A, T366W/T394S, F405W/T394S and T366W/T366S_L368A_Y407V.

Heavy chain heterodimer formation may be promoted by using electrostatic interactions by substituting positively charged residues on the first CH3 region and negatively charged residues on the second CH3 region as described in US2010/0015133, US2009/0182127, US2010/028637 or US2011/0123532.

Other asymmetric mutations that can be used to promote heavy chain heterodimerization are L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in US2012/0149876 or US2013/0195849 (Zymeworks).

SEEDbody mutations involve substituting select IgG residues with IgA residues to promote heavy chai heterodimerization as described in US20070287170.

Other exemplary mutations that may be used are R409D_K370E/D399K_E357K, S354C_T366W/Y349C_T366S_L368A_Y407V, Y349C_T366W/S354C_T366S L368A_Y407V, T366K/L351D, L351K/Y349E, L351K/Y349D, L351K/L368E, L351Y_Y407A/T366A_K409F, L351Y_Y407A/T366V_K409F, K392D/D399K, K392D/E356K, K253E D282K_K322D/D239K_E240K_K292D, K392D_K409D/D356K_D399K as described in WO2007/147901, WO 2011/143545, WO2013157954, WO2013096291 and US2018/0118849.

Duobody® mutations (Genmab) are disclosed for example in U.S. Pat. No. 9,150,663 and US2014/0303356 and include mutations F405L/K409R, wild-type/F405L_R409K, T350I_K370T F405L/K409R, K370W/K409R, D399AFGHILMNRSTVWY/K409R, T366ADEFGHILMQVY/K409R, L368ADEGHNRSTVQ/K409AGRH, D399FHKRQ/K409AGRH, F405IKLSTVW/K409AGRH and Y407LWQ/K409AGRH.

Additional bispecific or multispecific structures into which the antigen binding domains that specifically bind the multiple myeloma neoantigen or multiple myeloma neoantigen/HLA complexes can be incorporated include Dual Variable Domain Immunoglobulins (DVD) (Int. Pat. Publ. No. WO2009/134776; DVDs are full length antibodies comprising the heavy chain having a structure VH1-linker-VH2-CH and the light chain having the structure VL1-linker-VL2-CL; linker being optional), structures that include various dimerization domains to connect the two antibody arms with different specificity, such as leucine zipper or collagen dimerization domains (Int. Pat. Publ. No. WO2012/022811, U.S. Pat. Nos. 5,932,448; 6,833,441), two or more domain antibodies (dAbs) conjugated together, diabodies, heavy chain only antibodies such as camelid antibodies and engineered camelid antibodies, Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer), IgG-like Bispecific (InnClone/Eli Lilly), Ts2Ab (MedImmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv)2-Fab (National Research Center for Antibody Medicine--China), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based, and domain antibodies, include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

### Alternative Scaffolds

Alternative scaffolds (also referred to as antibody mimetics) that specifically bind the multiple myeloma neoantigen or multiple myeloma neoantigen/HLA complexes may be generated using various scaffolds known in the art and described herein. Alternative scaffolds may be monobodies, designed to incorporate the fibronectin type III domain (Fn3) of fibronectin or tenascin as a protein scaffold (U.S. Pat. Nos. 6,673,901; 6,348,584) or synthetic FN3 domains such as tencon as described in U.S. Pat. Publ. No. 2010/0216708 and U.S. Pat. Pub. No. 2010/0255056. Additional alternative scaffolds comprise Adnectin™, an iMab, an Anticalin®, an EETI-II/AGRP, a Kunitz domain, a thioredoxin peptide aptamer, an Affibody®, a DARPin, an Affilin, a Tetranectin, a Fynomer, and an Avimer. Alternative scaffolds are single chain polypeptidic frameworks that contains a highly structured core associated with variable domains of high conformational tolerance allowing insertions, deletions, or other substitutions within the variable domains. Libraries introducing diversity to one or more variable domains, and in some instances to the structured core, may be generated using known protocols and the resulting libraries may be screened for binding to the neoantigen of the disclosure, and the identified binders may be further characterized for their specificity using known methods. Alternative scaffold may be derived from Protein A, in particular, the Z-domain thereof (affibodies), ImmE7 (immunity proteins), BPTI/APPI (Kunitz domains), Ras-binding protein AF-6 (PDZ-domains), charybdotoxin (Scorpion toxin), CTLA-4, Min-23 (knottins), lipocalins (anticalins), neokarzinostatin, a fibronectin domain, an ankyrin consensus repeat domain, or thioredoxin (Skerra, A., “Alternative Non-Antibody Scaffolds for Molecular Recognition,” Curr. Opin. Biotechnol. 18:295-304 (2005); Hosse et al., “A New Generation of Protein Display Scaffolds for Molecular Recognition,” Protein Sci. 15:14-27 (2006); Nicaise et al., “Affinity Transfer by CDR Grafting on a Nonimmunoglobulin Scaffold,” Protein Sci. 13:1882-1891 (2004); Nygren and Uhlen, “Scaffolds for Engineering Novel Binding Sites in Proteins,” Curr. Opin. Struc. Biol. 7:463-469 (1997).

### Chimeric Antigen Receptors (CAR)

CARS may be generated that bind the multiple myeloma neoantigens or the multiple myeloma neoantigen/HLA complex by incorporating an antigen binding domain that specifically binds the multiple myeloma neoantigens or the multiple myeloma neoantigen/HLA complex to the extracellular domain of the CAR. CARS are genetically engineered receptors. These engineered receptors can be readily inserted into and expressed by immune cells, including T cells in accordance with techniques known in the art. With a CAR, a single receptor can be programmed to both recognize a specific antigen and, when bound to that antigen, activate the immune cell to attack and destroy the cell bearing that antigen. When these antigens exist on tumor cells, an immune cell that expresses the CAR can target and kill the tumor cell.

The CAR typically comprises an extracellular domain that binds the antigen (e.g. the multiple myeloma neoantigen or the multiple myeloma neoantigen/HLA complex), an optional linker, a transmembrane domain, and a cytosolic domain comprising a costimulatory domain and/or a signaling domain.

The extracellular domain of the CAR may contain any polypeptide that specifically binds the desired antigen (e.g. multiple myeloma neoantigen). The extracellular domain may comprise a scFv, a portion of an antibody or an alternative scaffold. The CARs may also be engineered to bind two or more desired antigens that may be arranged in tandem and separated by linker sequences. For example, one or more domain antibodies, scFvs, llama VHH antibodies or other VH only antibody fragments may be organized in tandem via a linker to provide bispecificity or multispecificity to the CAR.

The transmembrane domain of the CAR may be derived from the transmembrane domain of CD8, an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CDI la, CD18), ICOS (CD278), 4-1 BB (CD137), 4-1 BBL, GITR, CD40, BAFFR, HVEM (LIGHTR), SLAMF7, NKp80 (KLRFI), CD160, CD1 9, IL2R beta, IL2R gamma, IL7R a, ITGA1, VLA1, CD49a, ITGA4, IA4, CD49D, ITGA6, VLA-6, CD49f, ITGAD, CDI Id, ITGAE, CD103, ITGAL, CDI 1a, LFA-1, ITGAM, CDI 1b, ITGAX, CDI 1c, ITGB1, CD29, ITGB2, CD1 8, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

The intracellular costimulatory domain of CAR may be derived from the intracellular domains of one or more co-stimulatory molecules. Co-stimulatory molecules are well-known cell surface molecules other than antigen receptors or Fc receptors that provide a second signal required for efficient activation and function of T lymphocytes upon binding to antigen. Exemplary co-stimulatory domains that can be used in CARs are intracellular domains of 4-1BB, CD2, CD7, CD27, CD28, CD30, CD40, CD54 (ICAM), CD83, CD134 (OX40), CD150 (SLAMF1), CD152 (CTLA4), CD223 (LAG3), CD270 (HVEM), CD278 (ICOS), DAP10, LAT, NKD2C SLP76, TRIM, BTLA, GITR, CD226, HVEM, and ZAP70.

The intracellular signaling domain of the CAR may be derived from the signaling domains of for example CD3ζ, CD3ε, CD22, CD79a, CD66d, CD39 DAP10, DAP12, Fc epsilon receptor I gamma chain (FCER1G), FcRβ, CD3δ, CD3γ, CD5, CD226, or CD79B. “Intracellular signaling domain” refers to the part of the CAR polypeptide that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited following antigen binding to the extracellular CAR domain.

The optional linker of the CAR positioned between the extracellular domain and the transmembrane domain may be a polypeptide of about 2 to 100 amino acids in length. The linker may include or be composed of flexible residues such as glycine and serine so that the adjacent protein domains are free to move relative to one another. Longer linkers may be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. The linker may also be derived from a hinge region or portion of the hinge region of any immunoglobulin. Non-limiting examples of linkers include a part of human CD8α chain, partial extracellular domain of CD28, FcyRllla receptor, IgG, IgM, IgA, IgD, IgE, an Ig hinge, or functional fragment thereof.

Exemplary CARs that may be used are for example CAR that contains an extracellular domain that binds the multiple myeloma neoantigen of the disclosure, CD8 transmembrane domain and CD3ζ signaling domain. Other exemplary CARs contain an extracellular domain that binds the multiple myeloma neoantigen of the disclosure, CD8 or CD28 transmembrane domain, CD28, 41BB or OX40 costimulatory domain and CD3ζ signaling domain.

The CARs are generated by standard molecular biology techniques. The extracellular domain that binds the desired antigen may be derived from antibodies or their antigen binding fragments generated using the technologies described herein.

### T Cell Receptor (TCR)

TCRs may be generated that bind the multiple myeloma neoantigen/HLA complexes. The TCRs may be identified based on T cell binding to the multiple myeloma neoantigen/HLA complex, either in-vivo or as an in-vitro system, for example, as a multimeric complex of neoantigen bound

HLA molecules, isolating the T cell and sequencing the TCR expressed in the T cells. The identified TCRs may be identified from αβ T cells or γδ T cells. The identified TCRs may be further engineered to improve their affinity, stability, solubility or the like. TCRs may be affinity matured utilizing the same technologies utilized to affinity mature immunoglobulins. TCRs may be expressed as soluble TCRs which have been cysteine stabilized, they can be stabilized by engineering mutations onto α/β interaction surface, for example G192R on α chain and R208G on β chain. TCRs may also be stabilized by engineering cysteine residues which form disulfide bonds into TCR constant domain, by introducing mutations into the hydrophobic core, such as at positions 11, 13,19, 21, 53, 76, 89, 91 or 94 of α chain, utilizing domain swaps including swaps between α and β chain V domains, transmembrane domains or constant domains as described in U.S. Pat. No. 7,329,731, 7,871,817B2, 7,569,664, 9,133,264, 9,624,292, US20120252742A1, US2016/0130319, EP3215164A1, EP3286210A1, WO2017091905A1 or U.S. Pat. No. 9,884,075.

### Cells Expressing the CARs or the TCRs of the Disclosure

Cells expressing the CARs or the TCRs that specifically bind the multiple myeloma neoantigens of the disclosure of the multiple myeloma neoantigen/HLA complexes of the disclosure are within the scope of the disclosure. The disclosure also provides isolated cells comprising the CAR of the disclosure or the TCR of the disclosure. In some embodiments, the isolated cells are transduced with the CAR or the TCR of the disclosure, resulting in constitutive expression of the CAR or the TCR of the disclosure on the surface of the cell. The cells expressing the CAR or the TCR of the disclosure may further be engineered to express one or more co-stimulatory molecules. Exemplary co-stimulatory molecules are CD28, ICOS, LIGHT, GITR, 4-1BB and OX40. The cells expressing the CAR or the TCR of the disclosure may further be engineered to produce one or more cytokines or chemokines or proinflammatory mediators, such as TNFα, IFNγ, IL-2, IL-3, IL-6, IL-7, IL-11, IL-12, IL-15, IL-17 or IL-21. The cells may have their endogenous TCR locus and/or HLA locus inactivated using known gene editing technologies. In some embodiment, the cell comprising the CAR or the TCR of the disclosure is a T cell, a natural killer (NK) cell, a cytotoxic T lymphocyte (CTL), a regulatory T cell (Treg), a human embryonic stem cell, a lymphoid progenitor cell, a T cell-precursor cell, or a pluripotent stem cell or induced pluripotent stem cell (iPSC) from which lymphoid cells may be differentiated.

In some embodiments, the isolated cell comprising the CAR or the TCR of the disclosure is a T cell. The T cell may be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from any source, including to bone marrow, blood, lymph node, thymus, or other tissues or fluids. T cells may also be enriched for or purified. The T cell may be a human T cell. The T cell may be a T cell isolated from a human. The T cell can be any type of T cell and may be of any developmental stage, including, CD4⁺CD8⁺ double positive T cells, CD8⁺ T cells (e.g., cytotoxic T cells), CD4⁺ helper T cells, e.g., Th1 and Th2 cells,1 peripheral blood mononuclear cells (PBMCs), peripheral blood leukocytes (PBLs), tumor infiltrating cells, memory T cells, naïve T cells, and the like. The T cell may be a CD8⁺ T cell or a CD4⁺ T cell. The T cell may be an αβ T cell or a γδ T cell.

In some embodiments, the isolated cell comprising the CAR of the disclosure or the TCR of the disclosure is a NK cell.

In some embodiments, the isolated cell comprising the CAR of the disclosure or the TCR of the disclosure is an αβ T cell.

In some embodiments, the isolated cell comprising the CAR of the disclosure or the TCR of the disclosure is a γδ T cell.

In some embodiments, the isolated cell comprising the CAR of the disclosure or the TCR of the disclosure is a CTL.

In some embodiments, the isolated cell comprising the CAR of the disclosure or the TCR of the disclosure is a human embryonic stem cell.

In some embodiments, the isolated cell comprising the CAR of the disclosure or the TCR of the disclosure is a lymphoid progenitor cell.

In some embodiments, the isolated cell comprising the CAR of the disclosure or the TCR of the disclosure is a pluripotent stem cell.

In some embodiments, the isolated cell comprising the CAR of the disclosure or the TCR of the disclosure is an induced pluripotent stem cell (iPSC).

The cells of the disclosure may be generated by introducing a lentiviral vector comprising a desired CAR or TCR into the cells using known methods. The cells of the disclosure are able to replicate in vivo resulting in long-term persistence that can lead to sustained tumor control.

Conjugates with Cytotoxic Agents, Drugs, Detectable Labels, and the Like

The polypeptides, the heterologous polypeptide and the proteinaceous molecules binding them may be conjugated to a cytotoxic agent, therapeutics, detectable labels and the like. These molecules are referred herein to immunoconjugates. The immunoconjugates comprising the multiple myeloma neoantigens may be used to detect, deliver payload or kill cells expressing a HLA molecule that binds the multiple myeloma neoantigen. The immunoconjugates comprising the antibodies, antigen binding fragments or alternative scaffolds which specifically bind the multiple myeloma neoantigen or the multiple myeloma neoantigen/HLA complex may be used to detect, deliver payload or kill cells that express the multiple myeloma neoantigen on their surface in the context or a larger protein or in complex with HLA, or detect intracellular multiple myeloma neoantigens after lysis of the cells.

In some embodiments, the immunoconjugate comprises a detectable label.

In some embodiments, the immunoconjugate comprises a cytotoxic agent.

In some embodiments, the immunoconjugate comprises a therapeutic.

Detectable label includes compositions that can be visualized via spectroscopic, photochemical, biochemical, immunochemical, or chemical means. Detectable labels may also include cytotoxic agents, an cytotoxic agents may include detectable labels.

Exemplary detectable labels include radioactive isotopes, magnetic beads, metallic beads, colloidal particles, fluorescent dyes, electron-dense reagents, enzymes (for example, as commonly used in an ELISA), biotin, digoxigenin, haptens, luminescent molecules, chemiluminescent molecules, fluorochromes, fluorophores, fluorescent quenching agents, colored molecules, radioactive isotopes, scintillates, avidin, streptavidin, protein A, protein G, antibodies or fragments thereof, polyhistidine, Ni²⁺, Flag tags, myc tags, heavy metals, enzymes, alkaline phosphatase, peroxidase, luciferase, electron donors/acceptors, acridinium esters, and colorimetric substrates.

A detectable label may emit a signal spontaneously, such as when the detectable label is a radioactive isotope. In other cases, the detectable label emits a signal as a result of being stimulated by an external field.

Exemplary radioactive isotopes may be γ-emitting, Auger-emitting, β-emitting, an alpha-emitting or positron-emitting radioactive isotope. Exemplary radioactive isotopes include ³H, ¹¹C, ¹³C, ¹⁵N, ¹⁸F, ¹⁹F, ⁵⁵Co, ⁵⁷Co, ⁶⁰CO, ⁶¹Cu, ⁶²Cu, ⁶⁴Cu, ⁶⁷Cu, ⁶⁸Ga, ⁷²AS, ⁷⁵Br, ⁸⁶Y, ⁸⁹Zr, ⁹⁰Sr, ^{94m}Tc, ^{99m}TC, ¹¹⁵In, ¹²³I, ¹²⁴I, ¹²⁵I, ¹³¹I, ²¹¹At, ²¹²Bi, ²¹³Bi, ²²³Ra, ²²⁶Ra, ²²⁵Ac and ²²⁷Ac.

Exemplary metal atoms are metals with an atomic number greater than 20, such as calcium atoms, scandium atoms, titanium atoms, vanadium atoms, chromium atoms, manganese atoms, iron atoms, cobalt atoms, nickel atoms, copper atoms, zinc atoms, gallium atoms, germanium atoms, arsenic atoms, selenium atoms, bromine atoms, krypton atoms, rubidium atoms, strontium atoms, yttrium atoms, zirconium atoms, niobium atoms, molybdenum atoms, technetium atoms, ruthenium atoms, rhodium atoms, palladium atoms, silver atoms, cadmium atoms, indium atoms, tin atoms, antimony atoms, tellurium atoms, iodine atoms, xenon atoms, cesium atoms, barium atoms, lanthanum atoms, hafnium atoms, tantalum atoms, tungsten atoms, rhenium atoms, osmium atoms, iridium atoms, platinum atoms, gold atoms, mercury atoms, thallium atoms, lead atoms, bismuth atoms, francium atoms, radium atoms, actinium atoms, cerium atoms, praseodymium atoms, neodymium atoms, promethium atoms, samarium atoms, europium atoms, gadolinium atoms, terbium atoms, dysprosium atoms, holmium atoms, erbium atoms, thulium atoms, ytterbium atoms, lutetium atoms, thorium atoms, protactinium atoms, uranium atoms, neptunium atoms, plutonium atoms, americium atoms, curium atoms, berkelium atoms, californium atoms, einsteinium atoms, fermium atoms, mendelevium atoms, nobelium atoms, or lawrencium atoms.

In some embodiments, the metal atoms may be alkaline earth metals with an atomic number greater than twenty.

In some embodiments, the metal atoms may be lanthanides.

In some embodiments, the metal atoms may be actinides.

In some embodiments, the metal atoms may be transition metals.

In some embodiments, the metal atoms may be poor metals.

In some embodiments, the metal atoms may be gold atoms, bismuth atoms, tantalum atoms, and gadolinium atoms.

In some embodiments, the metal atoms may be metals with an atomic number of 53 (i.e. iodine) to 83 (i.e. bismuth).

In some embodiments, the metal atoms may be atoms suitable for magnetic resonance imaging.

The metal atoms may be metal ions in the form of +1, +2, or +3 oxidation states, such as Ba²⁺, Bi³⁺, Cs⁺, Ca²⁺, Cr²⁺, Cr³⁺, Cr⁶⁺, Co²⁺, Co³⁺, Cu⁺, Cu²⁺, Cu³⁺, Ga³⁺, Gd³⁺, Au⁺, Au³⁺, Fe²⁺, Fe³⁺, F³⁺, Pb²⁺, Mn²⁺, Mn³⁺, Mn⁴⁺, Mn⁷⁺, Hg²⁺, Ni²⁺, Ni³⁺, Ag⁺, Sr²⁺, Sn²⁺, Sn⁴⁺, and Zn²⁺. The metal atoms may comprise a metal oxide, such as iron oxide, manganese oxide, or gadolinium oxide.

Suitable dyes include any commercially available dyes such as, for example, 5(6)-carboxyfluorescein, IRDye 680RD maleimide or IRDye 800CW, ruthenium polypyridyl dyes, and the like.

Suitable fluorophores are fluorescein isothiocyanate (FITC), fluorescein thiosemicarbazide, rhodamine, Texas Red, CyDyes (e.g., Cy3, Cy5, Cy5.5), Alexa Fluors (e.g., Alexa488, Alexa555, Alexa594; Alexa647), near infrared (NIR) (700-900 nm) fluorescent dyes, and carbocyanine and aminostyryl dyes.

The immunoconjugates comprising a detectable label may be used as an imaging agent.

In some embodiments, the cytotoxic agent is a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

In some embodiments, the cytotoxic agent is daunomycin, doxorubicin, methotrexate, vindesine, bacterial toxins such as diphtheria toxin, ricin, geldanamycin, maytansinoids or calicheamicin. The cytotoxic agent may elicit their cytotoxic and cytostatic effects by mechanisms including tubulin binding, DNA binding, or topoisomerase inhibition.

In some embodiments, the cytotoxic agent is an enzymatically active toxin such as diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinahs inhibitor, gelonin, mitogellin, restrictocin, phenomycin, enomycin, and the tricothecenes.

In some embodiments, the cytotoxic agent is a radionuclide, such as ²¹²Bi, ¹³¹I, ¹³¹In, ⁹⁰Y, and ¹⁸⁶Re.

In some embodiments, the cytotoxic agent is dolastatins or dolostatin peptidic analogs and derivatives, auristatin or monomethyl auristatin phenylalanine Exemplary molecules are disclosed in U.S. Pat Nos. 5,635,483 and 5,780,588. Dolastatins and auristatins have been shown to interfere with microtubule dynamics, GTP hydrolysis, and nuclear and cellular division (Woyke et al (2001) Antimicrob Agents and Chemother. 45(12):3580-3584) and have anticancer and antifungal activity. The dolastatin or auristatin drug moiety may be attached to the antibody of the invention through the N (amino) terminus or the C (carboxyl) terminus of the peptidic drug moiety (WO02/088172), or via any cysteine engineered into the antibody.

The immunoconjugates may be made using known methods.

In some embodiments, the detectable label is complexed with a chelating agent.

The detectable label, cytotoxic agent or therapeutic may be linked directly, or indirectly via a linker, to the polypeptides, the heterologous polypeptides or the proteinaceous molecules that bind the polypeptides or the heterologous polypeptides. Suitable linkers are known in the art and include, for example, prosthetic groups, non-phenolic linkers (derivatives of N-succimidyl-benzoates; dodecaborate), chelating moieties of both macrocyclics and acyclic chelators, such as derivatives of 1,4,7,10-tetraazacyclododecane-1,4,7,10,tetraacetic acid (DOTA), derivatives of diethylenetriaminepentaacetic avid (DTPA), derivatives of S-2-(4-Isothiocyanatobenzyl)-1,4,7-triazacyclononane-1,4,7-triacetic acid (NOTA) and derivatives of 1,4,8,11-tetraazacyclodocedan-1,4,8,11-tetraacetic acid (TETA), N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene) and other chelating moieties. Suitable peptide linkers are well known.

### Methods of Treatment, Uses and Administration of any of the Compositions Herein

Provided herein are methods for treating a subject with the compositions disclosed herein. The methods provided herein comprise administering a composition comprising any of the polynucleotides, polypeptides, vectors, and recombinant viruses, of the disclosure. The composition comprising polynucleotides, polypeptides, vectors, recombinant viruses, and administration regimens of the disclosure may be used to treat, prevent or reduce the risk of a clinical condition.

In some embodiments, the clinical condition is multiple myeloma.

“Multiple myeloma” refers to a malignant disorder of plasma cells characterized by uncontrolled and progressive proliferation of one or more malignant plasma cells. The abnormal proliferation of plasma (myeloma) cells causes displacement of the normal bone marrow leading to dysfunction in hematopoietic tissue and destruction of the bone marrow architecture, resulting in progressive morbidity and eventual mortality.

In a majority of patients, multiple myeloma evolves from premalignant, asymptomatic plasma cell disorders such as non IgM monoclonal gammopathy of undetermined significance (MGUS) or smoldering multiple myeloma (SMM), which are characterized by monoclonal plasma cell proliferation in the bone marrow and absence of end-organ damage such as renal failure, anemia, and lytic bone lesions. Smoldering multiple myeloma accounts for 13% to 15% of all myeloma patients and progresses to symptomatic multiple myeloma at a rate of 10% per year for the first 5 years, decreasing to 3% per year over the following 5 years.

In some embodiments, multiple myeloma is non IgM monoclonal gammopathy of undetermined significance (MGUS) or smoldering multiple myeloma (SMM).

In some embodiments, multiple myeloma is newly diagnosed multiple myeloma.

In some embodiments, multiple myeloma is relapsed, refractory, or both relapsed and refractory.

In some embodiments, the multiple myeloma is relapsed or refractory to treatment with an anti-CD38 antibody, a glutamic acid derivative, a proteasome inhibitor, an alkylating agent, a microtubule inhibitor, lenalinomide, bortezomib, pomalidomide, carfilzomib, elotuzumab, ixazomib, melphalan or thalidomide, or any combination thereof.

In some embodiments, the multiple myeloma is relapsed or refractory to treatment with an anti-CD38 antibody. In some embodiments, the multiple myeloma is relapsed or refractory to treatment with glutamic acid derivative. In some embodiments, the multiple myeloma is relapsed or refractory to treatment with a proteasome inhibitor. In some embodiments, the multiple myeloma is relapsed or refractory to treatment with an alkylating agent. In some embodiments, the multiple myeloma is relapsed or refractory to treatment with a microtubule agent. In some embodiments, the multiple myeloma is relapsed or refractory to treatment with lenalinomide. In some embodiments, the multiple myeloma is relapsed or refractory to treatment with bortezomib. In some embodiments, the multiple myeloma is relapsed or refractory to treatment with pomalidomide. In some embodiments, the multiple myeloma is relapsed or refractory to treatment with carfilzomib. In some embodiments, the multiple myeloma is relapsed or refractory to treatment with elotozumab. In some embodiments, the multiple myeloma is relapsed or refractory to treatment with ixazomib. In some embodiments, the multiple myeloma is relapsed or refractory to treatment with melphalan. In some embodiments, the multiple myeloma is relapsed or refractory to treatment with or thalidomide.

In some embodiments, the subject has high-risk multiple myeloma. Subjects can be classified as “high risk” if they have one or more of the following cytogenetic abnormalities: t(4; 14)(p16; q32), t(14; 16)(q32; q23), or del17p. Thus, the subject having high-risk multiple myeloma can have one or more chromosomal abnormalities comprising:
a. t(4; 14)(p16; q32);
b. t(14; 16)(q32; q23);
c. del17p;
d. t(4; 14)(p16; q32) and t(14; 16)(q32; q23);
e. t(4; 14)(p16; q32) and del17p;
f. t(14; 16)(q32; q23) and del17p; or
g. t(4; 14)(p16; q32), t(14; 16)(q32; q23) and del17p.

The cytogenetic abnormalities can be detected for example by fluorescent in situ hybridization (FISH). In chromosomal translocations, an oncogene is translocated to the IgH region on chromosome 14q32, resulting in dysregulation of these genes. t(4; 14)(p16; q32) involves translocation of fibroblast growth factor receptor 3 (FGFR3) and multiple myeloma SET domain containing protein (MMSET) (also called WHSC1/NSD2), and t(14; 16)(q32; q23) involves translocation of the MAF transcription factor C-MAF. Deletion of 17p (del17p) involves loss of the p53 gene locus.

In some embodiments, the subject is treatment naive.

In some embodiments, the subject has received high dose chemotherapy (HDC) and stem cell transplant (SCT).

In some embodiments, the subject has an elevated level of monoclonal paraprotein (M-protein). M-protein levels may also be compared to post-high dose chemotherapy (HDC) and stem cell transplant (SCT) levels. Subjects afflicted with multiple myeloma satisfy the CRAB (calcium elevation, renal insufficiency, anemia and bone abnormalities) criteria, and have clonal bone marrow plasma cells ≥10% or biopsy-proven bony or extramedullary plasmacytoma, and measurable disease.

Measurable disease is defined by any of the following;
IgG myeloma: Serum monoclonal paraprotein (M-protein) level ≥1.0 g/dL or urine M-protein level ≥200 mg/24 hours; or
IgA, IgM, IgD, or IgE multiple myeloma: serum M-protein level ≥0.5 g/dL or urine M-protein level ≥200 mg/24 hours; or
Light chain multiple myeloma without measurable disease in serum or urine: Serum immunoglobulin free light chain ≥10 mg/dL and abnormal serum immunoglobulin kappa lambda free light chain ratio.
   CRAB criteria is defined by the following:
Hypercalcemia: serum calcium >0.25 mM/L (>1 mg/dL) higher than the upper limit of the normal range [ULN] or >2.75 mM/L (>11 mg/dL)
Renal insufficiency: creatinine clearance <40mL/min or serum creatinine >177 μM/L (>2 mg/dL)
Anemia: hemoglobin >2 g/dL below the lower limit of normal or hemoglobin <10 g/dL
Bone lesions: one or more osteolytic lesions on skeletal radiography, CT, or PET-CT.

Response to treatment may be assessed using International Myeloma Working Group (IMWG) uniform response criteria recommendations (International Uniform Response Criteria Consensus Recommendations) as shown in Table 1.

TABLE 1

| Response | Response Criteria |
|---|---|
| Stringent | CR as defined below, plus |
| complete | Normal FLC ratio, and |
| Response (sCR) | Absence of clonal PCs by immunohistochemistry, immunofluorescence or |
| | 2- to 4-color flow cytometry |
| Complete | Negative immunofixation on the serum and urine, and |
| response (CR) | Disappearance of any soft tissue plasmacytomas, and |
| | <5% PCs in bone marrow |
| Very good | Serum and urine M-component detectable by immunofixation but not on |
| partial | electrophoresis, |
| Response | or |
| (VGPR) | ≥90% reduction in serum M-protein plus urine M-protein <100 mg/24 hours |
| Partial response | ≥50% reduction of serum M-protein and reduction in 24-hour urinary M- |
| (PR) | protein by ≥90% or to <200 mg/24 hours |
| | If the serum and urine M-protein are not measurable, a decrease of ≥50% in |
| | the difference between involved and uninvolved FLC levels is required in |
| | place of the M-protein criteria |
| | If serum and urine M-protein are not measurable, and serum free light assay |
| | is also not measurable, ≥50% reduction in bone marrow PCs is required in |
| | place of M-protein, provided baseline bone marrow plasma cell percentage |
| | was ≥30% |
| | In addition to the above criteria, if present at baseline, a ≥50% reduction in |
| | the size of soft tissue plasmacytomas is also required. |
| Stable disease | Not meeting criteria for CR, VGPR, PR, or progressive disease |
| (SD) | |
| Progressive | Increase of 25% from lowest response value in any one of the following: |
| disease (PD) | Serum M-component (absolute increase must be ≥0.5 g/dL), |
| | Urine M-component (absolute increase must be ≥200 mg/24 hours), |
| | Only in subjects without measurable serum and urine M-protein levels: the |
| | difference between involved and uninvolved FLC levels (absolute increase |
| | must be >10 mg/dL) |
| | Only in subjects without measurable serum and urine M-protein levels and |
| | without measurable disease by FLC levels, bone marrow PC percentage |
| | (absolute percentage must be ≥10%) |
| | Bone marrow plasma cell percentage: the absolute percentage must be >10% |
| | Definite development of new bone lesions or soft tissue plasmacytomas or |
| | definite increase in the size of existing bone lesions or soft tissue |
| | plasmacytomas |
| | Development of hypercalcemia (corrected serum calcium >11.5 mg/dL) that |
| | can be attributed solely to the PC proliferative disorder |

EBMT = European Group for Blood and Marrow Transplantation; FLC = free light chain; PC = plasma cell

Provided herein, are methods for treating, preventing or reducing the risk of multiple myeloma in a subject comprising admistering the various compositions of the disclosure that can be used to introduce the multiple myeloma neoantigens of the disclosure into a subject, e.g. the polynucleotides, the heterologous polynucleotides, the polypeptides, the heterologous polypeptides, the vectors, the recombinant viruses and vaccines of the disclosure may be used to treat multiple myeloma in a subject. Additionally, the proteinaceous molecules that bind the multiple myeloma neoantigens of the disclosure can be used in the methods of the disclosure.

The disclosure also provides methods for inducing an immune response in a subject comprising admistering the various compositions of the disclosure that can be used to introduce the multiple myeloma cancer neoantigens of the disclosure into the subject, e.g. the polynucleotides, the heterologous polynucleotides, the polypeptides, the heterologous polypeptides, the vectors, the recombinant viruses and vaccines of the disclosure.

In some embodiments, the multiple myeloma neoantigens identified herein are present at a frequency of at least about 1% or more, about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 16% or more about 17% or more, about 18% or more, about 19% or more, about 20% or more, about 21% or more, about 22% or more, about 23% or more, about24% or more, about 25% or more, about 26% or more, about 27% or more, about 28% or more, about 29% or more, about 30% or more, about 35% or more, about 40% or more, about 45% or more, about 50% or more, about 55% or more, about 60% or more, about 65% or more or about 70% or more in a population of subjects having the multiple myeloma.

In some embodiments, the method of treating, preventing, reducing a risk of onset or delaying the onset of multiple myeloma cancer in a subject comprises administering to the subject in need thereof any of the compositions disclosed herein, and wherein the administration comprises one or more administrations of the composition.

In some embodiments, the method of inducing an immune response comprises administering to the subject in need thereof any of the compositions disclosed herein, and wherein the administration comprises one or more administrations of the composition.

In any of the methods disclosed herein, the composition that is administered to a subject may comprise a recombinant virus selected from adenovirus, alphavirus, poxvirus, adeno-associated virus, retrovirus, or may comprise a self-replicating RNA, or a combination thereof.

In some embodiments, the recombinant virus comprises the multiple myeloma cancer neoantigens of the disclosure, e.g. the polynucleotides, the heterologous polynucleotides, the polypeptides, the heterologous polypeptides and the vectors, of the disclosure.

In some embodiments, the virus or recombinant virus is selected from Ad26, MVA, GAd20, and combinations thereof.

In some embodiments, the vaccine comprises the rAd26 of the disclosure.

In some embodiments, the composition comprises the rMVA of the disclosure.

In some embodiments, the composition comprises the rGAd of the disclosure.

In some embodiments, the composition comprises the rGAd20 of the disclosure.

In some embodiments, the composition comprises the rCh20 of the disclosure.

In some embodiments, the composition comprises the rAd26 of the disclosure and rMVA of the disclosure.

In some embodiments, the composition comprises the rGAd20 of the disclosure and rMVA of the disclosure.

In some embodiments, the composition comprises the self-replicating RNA molecule of the disclosure and rMVA of the disclosure.

In some embodiments, the composition comprises the heterologous polypeptide of the disclosure.

In some embodiments, the composition comprises the heterologous polynucleotide of the disclosure.

In some embodiments, the composition comprises the polynucleotide of the disclosure.

In some embodiments, the composition comprises the polypeptide of the disclosure.

### Second Admistration

In some embodiments, the methods disclosed herein comprise one or more administrations of the compositions provided in the disclosure. For example, the method comprises a first administration followed by a second administration, with a time period between the two administrations.

In some embodiments, the first administration and the second administration may comprise the same or different compositions. For example, the first administration may comprise a composition comprising a recombinant virus selected from Ad26, GAd20, or MVA or a self-replicating RNA molecule comprising a polynucleotide encoding for any of the polypeptide of the disclosure, or combination thereof. In some embodiments, the second administration may comprise a composition comprising a recombinant virus selected from Ad26, GAd20, or MVA or a self-replicating RNA molecule comprising a polynucleotide encoding for any of the polypeptides of the diclosure, or combination thereof.

In some embodiments, the first administration and the second administration are administered once in a lifetime of the subject. In some embodiments, first administration and the second administration are administered two or more times in the lifetime of the subject.

In some embodiments, the time period between the first administration and the second administration is about 1 week to about 2 weeks, about 1 week to about 4 weeks, about 1 week to about 6 weeks, about 1 week to about 8 weeks, about 1 week to about 12 weeks, about 1 week to about 20 weeks, about 1 week to about 24 weeks, or about 1 week to about 52 weeks.

In some embodiments, the time period between the first administration and the second administration is about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, or about 52 weeks.

In some embodiments, the time period between the first administration and the second administration is about 2 weeks.

In some embodiments, the time period between the first administration and the second administration is about 4 weeks.

In some embodiments, the first administration and the second administration constitute a cycle, and the treatment regime may include two or more cycles, each cycle being spaced apart by about 1 month, about 2 months, about 3 months, about 4 months, about 5 months, about 6 months, about 7 months, about 8 months, about 9 months, about 10 months, about 11 months, or about 12 months.

The following example is provided to further describe some of the embodiments disclosed herein. The examples are intended to illustrate, not to limit, the disclosed embodiments. In some embodiments, the first administration and second administration can comprise any combination of recombinant virus or self-replicating RNA molecule provided in Table 2 comprising a polynucleotide encoding one or more polypeptides of the disclosure, or any combination thereof.

TABLE 2
Recombinant vims and self-replicating RNA molecule
composition in first and second administration

| First administration | Second administration |
|---|---|
| Ad26 | MVA |
| Ad26 | GAd20 |
| Ad26 | Self-replicating RNA molecule |
| Ad26 | Ad26 |
| MVA | Ad26 |
| MVA | GAd20 |
| MVA | Self-replicating RNA molecule |
| MVA | MVA |
| GAd20 | Ad26 |
| GAd20 | MVA |
| GAd20 | Self-replicating RNA molecule |
| GAd20 | GAd20 |
| Self-replicating RNA molecule | Ad26 |
| Self-replicating RNA molecule | MVA |
| Self-replicating RNA molecule | GAd20 |
| Self-replicating RNA molecule | Self-replicating RNA molecule |

In some embodiments, the first administration and second administration can comprise a polynucleotide encoding for any polypeptide of the disclosure or combination thereof. In some embodiments, the first administration and second administration can comprise a polynucleotide encoding for any polypeptide selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, and 421, and combinations thereof. In some embodiments, the first administration and second administration can comprise a polynucleotide encoding two or more tandem repeats of any polypeptides of the disclosure.

In some embodiments, the first and the second administration may comprise a distinct recombinant virus.

In some embodiments, the first and the second administration comprise a recombinant virus comprising a polynucleotide encoding for a polypeptide of distinct amino acid sequence.

In some embodiments, a method of inducing an immune response or a method of treating, preventing, reducing a risk of onset or delaying the onset of multiple myeloma in a subject comprises a treatment cycle, wherein each cycle comprises:

a first administration comprising a first composition comprising a recombinant virus or self-replicating RNA molecule comprising a polynucleotide encoding one or more polypeptides of the disclosure or combination thereof, wherein the virus or recombinant virus is selected from Ad26, MVA, GAd20; and

a second administration comprising a second composition comprising a recombinant virus, or a self-replicating RNA molecule comprising a polynucleotide encoding for any polypeptide of the disclosure, or combination thereof, wherein the recombinant virus is selected from Ad26, MVA, GAd20.

### Third Administration

In some embodiments, any of the methods disclosed herein may further comprise a third administration. For example, the method may comprise a first administration, a second administration, followed by a third administration, with a time period between each administration.

In some embodiments, the first administration, second administration, and third administration may comprise the same or different compositions. For example, the first administration may comprise a composition comprising a recombinant virus selected from Ad26, GAd20, or MVA or a self-replicating RNA molecule comprising a polynucleotide encoding for any of the polypeptides of the composition or combination thereof. In some embodiments, the second administration may comprise a recombinant virus selected from Ad26, GAd20, or MVA or a self-replicating RNA molecule comprising a polynucleotide encoding for any of the polypeptides of the composition or combination thereof. In some embodiments, the third administration may comprise a composition comprising a recombinant virus selected from Ad26, GAd20, or MVA or a self-replicating RNA molecule comprising a polynucleotide encoding for any of the polypeptides of the composition or combination thereof.

In some embodiments, the first administration, second administration and third administration comprise a composition comprising a recombinant virus selected from Ad26, GAd20, MVA or a self-replicating RNA molecule comprising a polynucleotide encoding one or more polypeptides selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, and 421, and combinations thereof.

In some embodiments, the first, the second or the third administration comprise a polynucleotide encoding two or more tandem repeats of any polypeptides of the disclosure.

In some embodiments, the first, the second or the third administration may comprise a distinct recombinant virus.

In some embodiments, the first, the second or the third administration may comprise a recombinant virus comprising a polynucleotide encoding for a polypeptide of distinct amino acid sequence.

For example, the first administration may comprise a polynucleotide encoding one or more polypeptides selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, and 421, and combinations thereof. In some embodiments, the second administration may comprise a polynucleotide encoding one or more polypeptides selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, and 421, and combination thereof. In some embodiments, the third administration may comprise a polynucleotide encoding one or more polypeptides selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, and 421, and combinations thereof.

In some embodiments, the first administration, the second administration, and the third administration are administered once in a lifetime of the subject. In some embodiments, the first, second, and third administration are administered two or more times in the lifetime of the subject.

In some embodiments, the time period between the second administration and the third administration is about 1 week to about 2 weeks, about 1 week to about 4 weeks, about 1 week to about 6 weeks, about 1 week to about 8 weeks, about 1 week to about 12 weeks, about 1 week to about 20 weeks, about 1 week to about 24 weeks, or about 1 week to about 52 weeks.

In some embodiments, the time period between the second administration and the third administration is about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, or about 52 weeks.

In some embodiments, the time period between the second administration and the third administration is about 6 weeks.

In some embodiments, the time period between the second administration and the third administration is about 8 weeks.

In some embodiments, the first administration, second administration, and third administration together constitute a cycle, and the treatment regime may include two or more cycles, each cycle being spaced apart by about 1 month, about 2 months, about 3 months, about 4 months, about 5 months, about 6 months, about 7 months, about 8 months, about 9 months, about 10 months, about 11 months, or about 12 months.

The following examples are provided to further describe some of the embodiments disclosed herein. The first, second, and third administrations used in the methods disclosed herein can comprise any combination of the epitopes and compositions provided in Table 3.

TABLE 3
Recombinant vims and self-replicating RNA molecule composition
in first, second and third administration

| First administration | Second administration | Third administration |
|---|---|---|
| Ad26 | Ad26 | Ad26 |
| Ad26 | Ad26 | MVA |
| Ad26 | Ad26 | GAd20 |
| Ad26 | Ad26 | Self-replicating RNA |
| | | molecule |
| Ad26 | MVA | Ad26 |
| Ad26 | MVA | MVA |
| Ad26 | MVA | GAd20 |
| Ad26 | MVA | Self-replicating RNA |
| | | molecule |
| Ad26 | GAd20 | Ad26 |
| Ad26 | GAd20 | MVA |
| Ad26 | GAd20 | GAd20 |
| Ad26 | GAd20 | Self-replicating RNA |
| | | molecule |
| Ad26 | Self-replicating RNA | Ad26 |
| | molecule | |
| Ad26 | Self-replicating RNA | MVA |
| | molecule | |
| Ad26 | Self-replicating RNA | GAd20 |
| | molecule | |
| Ad26 | Self-replicating RNA | Self-replicating RNA |
| | molecule | molecule |
| MVA | Ad26 | Ad26 |
| MVA | Ad26 | MVA |
| MVA | Ad26 | GAd20 |
| MVA | Ad26 | Self-replicating RNA |
| | | molecule |
| MVA | MVA | Ad26 |
| MVA | MVA | MVA |
| MVA | MVA | GAd20 |
| MVA | MVA | Self-replicating RNA |
| | | molecule |
| MVA | GAd20 | Ad26 |
| MVA | GAd20 | MVA |
| MVA | GAd20 | GAd20 |
| MVA | GAd20 | Self-replicating RNA |
| | | molecule |
| MVA | Self-replicating RNA | Ad26 |
| | molecule | |
| MVA | Self-replicating RNA | MVA |
| | molecule | |
| MVA | Self-replicating RNA | GAd20 |
| | molecule | |
| MVA | Self-replicating RNA | Self-replicating RNA |
| | molecule | molecule |
| GAd20 | Ad26 | Ad26 |
| GAd20 | Ad26 | MVA |
| GAd20 | Ad26 | GAd20 |
| GAd20 | Ad26 | Self-replicating RNA |
| | | molecule |
| GAd20 | MVA | Ad26 |
| GAd20 | MVA | MVA |
| GAd20 | MVA | GAd20 |
| GAd20 | MVA | Self-replicating RNA |
| | | molecule |
| GAd20 | GAd20 | Ad26 |
| GAd20 | GAd20 | MVA |
| GAd20 | GAd20 | GAd20 |
| GAd20 | GAd20 | Self-replicating RNA |
| | | molecule |
| GAd20 | Self-replicating RNA | Ad26 |
| | molecule | |
| GAd20 | Self-replicating RNA | MVA |
| | molecule | |
| GAd20 | Self-replicating RNA | GAd20 |
| | molecule | |
| GAd20 | Self-replicating RNA | Self-replicating RNA |
| | molecule | molecule |
| Self-replicating RNA | Ad26 | Ad26 |
| molecule | | |
| Self-replicating RNA | Ad26 | MVA |
| molecule | | |
| Self-replicating RNA | Ad26 | GAd20 |
| molecule | | |
| Self-replicating RNA | Ad26 | Self-replicating RNA |
| molecule | | molecule |
| Self-replicating RNA | MVA | Ad26 |
| molecule | | |
| Self-replicating RNA | MVA | MVA |
| molecule | | |
| Self-replicating RNA | MVA | GAd20 |
| molecule | | |
| Self-replicating RNA | MVA | Self-replicating RNA |
| molecule | | molecule |
| Self-replicating RNA | GAd20 | Ad26 |
| molecule | | |
| Self-replicating RNA | GAd20 | MVA |
| molecule | | |
| Self-replicating RNA | GAd20 | GAd20 |
| molecule | | |
| Self-replicating RNA | GAd20 | Self-replicating RNA |
| molecule | | molecule |
| Self-replicating RNA | Self-replicating RNA | Ad26 |
| molecule | molecule | |
| Self-replicating RNA | Self-replicating RNA | MVA |
| molecule | molecule | |
| Self-replicating RNA | Self-replicating RNA | GAd20 |
| molecule | molecule | |
| Self-replicating RNA | Self-replicating RNA | Self-replicating RNA |
| molecule | molecule | molecule |

In some embodiments, a method of inducing an immune response or a method of treating, preventing, reducing a risk of onset or delaying the onset of multiple myeloma in a subject comprises a treatment cycle, wherein each cycle comprises:
a first administration comprising a first composition comprising a recombinant virus, or self-replicating RNA molecule comprising a polynucleotide encoding one or more polypeptides of the disclosure, wherein the recombinant virus is selected from Ad26, MVA, GAd20; and
a second administration comprising a scond composition comprising a recombinant virus or self-replicating RNA molecule comprising a polynucleotide encoding one or more polypeptides of the disclosure, wherein the recombinant virus is selected from Ad26, MVA, GAd20; and
a third administration comprising a third composition comprising a recombinant virus or self-replicating RNA molecule comprising a polynucleotide encoding one or more polypeptides of the disclosure, wherein the recombinant virus is selected from Ad26, MVA, GAd20.

### Fourth Administration

In some embodiments, any of the methods disclosed herein may further comprise a fourth administration. For example, the method may comprise a first administration, a second administration, a third administration, and a fourth administration, with a time period between each administration. In some embodiments, the first administration, second administration, third administration, and fourth administration may comprise the same or different compositions. For example, the fourth administration may comprise a composition comprising a recombinant virus selected from Ad26, GAd20, or MVA or a self-replicating RNA molecule encoding one or more polypeptides of the disclosure.

In some embodiment the first administration, the second administration, the third administration, and the fourth administration comprise a composition comprising a recombinant virus selected from Ad26, GAd20, or MVA or a self-replicating RNA molecule encoding one or more polypeptide selected from the group consisting of SEQ ID NO 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, and 421, and combinations thereof.

In some embodiments, the first, the second, the third, or the fourth administration comprise a polynucleotide encoding two or more tandem repeats of any polypeptides of the disclosure.

In some embodiments, the first, the second, the third, or the fourth administration may comprise a distinct recombinant virus.

In some embodiments, the first, the second, the third or the fourth administration may comprise a recombinant virus comprising a polynucleotide encoding for a polypeptide of distinct amino acid sequence.

In some embodiments, the first administration, the second administration, the third administration, and the fourth administration are administered once in a lifetime of the subject. In some embodiments, the first, second, third, and the fourth administration are administered two or more times in the lifetime of the subject.

In some embodiments, the time period between the third administration and the fourth administration is about 1 week to about 2 weeks, about 1 week to about 4 weeks, about 1 week to about 6 weeks, about 1 week to about 8 weeks, about 1 week to about 12 weeks, about 1 week to about 20 weeks, about 1 week to about 24 weeks, or about 1 week to about 52 weeks.

In some embodiments, the time period between the third administration and the fourth administration is about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, or about 52 weeks.

In some embodiments, the time period between the third administration and the fourth administration is about 4 weeks.

In some embodiments, the time period between the third administration and the fourth administration is about 8 weeks.

In some embodiments, the first administration, second administration, third administration, and the fourth administration together constitute a cycle, and the treatment regime may include two or more cycles, each cycle being spaced apart by about 1 month, about 2 months, about 3 months, about 4 months, about 5 months, about 6 months, about 7 months, about 8 months, about 9 months, about 10 months, about 11 months, or about 12 months.

In some embodiments, a method of inducing an immune response or a method of treating, preventing, reducing a risk of onset or delaying the onset of multiple myeloma in a subject comprises a treatment cycle, wherein each cycle comprises:
a first administration comprising a first composition comprising a recombinant virus, or self-replicating RNA molecule comprising a polynucleotide encoding one or more polypeptides of the disclosure, wherein the recombinant virus is selected from Ad26, MVA, GAd20; and
a second administration comprising a second composition comprising a recombinant virus, or self-replicating RNA molecule comprising a polynucleotide encoding one or more polypeptides of the disclosure, wherein the recombinant virus is selected from Ad26, MVA, GAd20; and
a third administration comprising a third composition comprising a recombinant virus, or self-replicating RNA molecule comprising a polynucleotide encoding one or more polypeptides of the disclosure, wherein the recombinant virus is selected from Ad26, MVA, GAd20, or a self-replicating RNA molecule; and
a fourth administration comprising a fourth composition comprising a recombinant virus, or self-replicating RNA molecule comprising a polynucleotide encoding one or more polypeptides of the disclosure, wherein the recombinant virus is selected from Ad26, MVA, GAd20.

### Maintenance Administration

In some embodiments, the method further comprises administering to the subject a composition at regular intervals during the treatment cycles, and may continue even after the treatment cycles have ended. For example, the composition may be administered to a subject every month during the treatment regimen, and may continue for additional 6 months. In some embodiments, the composition may be administered between two treatment cycles. In some embodiments, the composition may be any of the compositions disclosed herein, such as a composition comprising a vector selected from Ad26 vector, GAd20 vector, MVA vector or self-replicating RNA molecule encoding the epitope sequences.

### Dose and Route of Administration

The compositions of the disclosure may be administered to a subject by a variety of routes such as subcutaneous, topical, oral and intramuscular. Administration of the compositions may be accomplished orally or parenterally. Methods of parenteral delivery include topical, intra-arterial (directly to the tissue), intramuscular, intradermal, subcutaneous, intramedullary, intrathecal, intraventricular, intravenous, intraperitoneal, or intranasal administration. The present disclosure also has the objective of providing suitable topical, oral, systemic and parenteral formulations for use in the methods of prophylaxis and treatment.

In some embodiments, intramuscular administration of the vaccine composition can be achieved by using a needle. An alternative is the use of a needleless injection device to administer the composition (using, e.g., Biojector™) or a freeze-dried powder containing the vaccine.

For intravenous, cutaneous or subcutaneous injection, or injection at the site of affliction, the vaccine composition may be in the form of a parenterally acceptable aqueous solution which is pyrogen-free and has suitable pH, isotonicity and stability. Those of skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required. A slow-release formulation may also be employed.

Typically, administration will have a prophylactic aim to generate an immune response against the multiple myeloma neoantigens before development of symptoms of multiple myeloma cancer.

The compositions of the disclosure are administered to a subject, giving rise to an immune response in the subject. The amount of the vaccine able to induce a detectable immune response is defined to be an “immunologically effective dose.” The compositions of the disclosure may induce a humoral as well as a cell-mediated immune response. In a typical embodiment the immune response is a protective immune response.

In some embodiments, the methods of treating, preventing, reducing a risk of onset or delaying the onset of multiple myeloma in a subject, comprise administering to the subject a therapeutically effective amount of one or more vaccines of the disclosure.

In some embodiments, the methods of treating, preventing, reducing a risk of onset or delaying the onset of multiple myeloma in a subject, comprise administering to the subject a therapeutically effective amount of one or more compositions of the disclosure.

In some embodiments, the method of creating an immunre response in a subject, comprise administering to the subject a immunologically therapeutically effective amount of one or more compositions of the disclosure.

In some embodiments, the method of treating, preventing, reducing a risk of onset or delaying the onset of multiple myeloma in a subject, comprises administering to the subject a therapeutically effective amount of a vaccine or composition comprising a polynucleotide encoding one or more polypeptide of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, thereby treating, preventing, reducing a risk of onset or delaying the onset of the multiple myeloma in the subject, wherein the administration comprises one or more administrations of the composition.

In any of the methods disclosed herein, the composition that is administered to a subject may comprise a recombinant virus selected from adenovirus, alphavirus, poxvirus, adeno-associated virus, retrovirus, or may comprise a self-replicating RNA, or a combination thereof. In some embodiments, the subject is suspected to have or is suspected to develop multiple myeloma.

The actual amount administered, and rate and time-course of administration, will depend on the nature and severity of what is being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners.

In some embodiments, the compositions comprising recombinant adenovirus is administered at a dose from about 1×10⁴ IFU (Infectious Unit) to about 1×10¹² IFU per dose, about 1×10⁴ IFU to about 1×10¹¹IFU per dose, about 1×10⁴ IFU to about 1×10¹⁰ IFU per dose, about 1×10⁴ IFU to about 1×10⁹ IFU per dose, about 1×10⁴ IFU to about 1×10⁸ IFU per dose, or about 1×10⁴ IFU to about 1×10⁶ IFU per dose.

In some embodiments, the compositions comprising recombinant adenovirus is administered at a dose from about 1×10⁶ VP (viral particles) to about 1×10¹⁴ VP per dose, about 1×10⁶ VP to about 1×10¹² VP per dose, about 1×10⁶ VP to about 1×10¹⁰ VP per dose, about 1×10⁶ VP to about 1×10⁸ VP per dose, or about 1×10⁶ VP to about 1×10⁷ VP per dose.

In some embodiments, a composition comprising recombinant Ad26 virus is administered at about 1×10¹⁰ IFU per dose. In some embodiments, a composition comprising recombinant Ad26 virus is administered at about 1×10¹¹IFU per dose. In some embodiments, a composition comprising recombinant Ad26 virus is administered at about 1×10¹⁰ VP per dose. In some embodiments, a composition comprising recombinant Ad26 virus is administered at about 1×10¹¹ VP per dose.

In some embodiments, a composition comprising recombinant GAd20 virus is administered at about 1×10⁸IFU per dose. In some embodiments, a composition comprising recombinant GAd20 virus is administered at about 1×10¹⁰ IFU per dose. In some embodiments, a composition comprising recombinant GAd20 virus is administered at about 1×10¹⁰ VP per dose. In some embodiments, a composition comprising recombinant GAd20 virus is administered at about 1×10¹¹ VP per dose.

In some embodiments, the compositions comprising recombinant poxvirus is administered at dose from about 1×10⁴ IFU (Infectious Unit) to about 1×10¹² IFU per dose, about 1×10⁴ IFU to about 1×10¹¹IFU per dose, about 1×10⁴ IFU to about 1×10¹⁰ IFU per dose, about 1×10⁴ IFU to about 1×10⁹ IFU per dose, about 1×10⁴ IFU to about 1×10⁸ IFU per dose, or about 1×10⁴ IFU to about 1×10⁶ IFU per dose.

In some embodiments, a composition comprising recombinant MVA virus is administered from about 1×10⁸IFU per dose. In some embodiments, a composition comprising recombinant MVA virus is administered from about 1×10¹⁰ IFU per dose.

In some embodiments, the compositions comprising self-replicating RNA molecule is administered at a dose from about 1 microgram to about 100 microgram, about 1 microgram to about 90 micrograms, about 1 microgram to about 80 microgram, about 1 microgram to about 70 micrograms, about 1 microgram to about 60 micrograms, about 1 microgram to about 50 micrograms, about 1 microgram to about 40 micrograms, about 1 microgram to about 30 micrograms, about 1 microgram to about 20 micrograms, about 1 microgram to about 10 micrograms, or about 1 microgram to about 5 micrograms of the self-replicating RNA molecule.

In one exemplary regimen, the composition comprising the adenovirus is administered (e.g., intramuscularly) in a volume ranging between about 100 μL to about 10 ml containing concentrations of about 10⁴ to 10¹² virus particles/ml. The adenovirus vector may be administered in a volume ranging between 0.25 and 1.0 ml, such as in a volume of 0.5 ml.

The adenovirus may be administered in an amount of about 10⁹ to about 10¹² viral particles (vp) to a human subject during one administration, more typically in an amount of about 10¹⁰ to about 10¹² vp.

In one exemplary regimen, the composition comprising the rMVA virus of the disclosure is administered (e.g., intramuscularly) in a volume ranging between about 100 μL to about 10 ml of saline solution containing a dose of about 1×10⁷ TCID₅₀ to 1×10⁹ TCID₅₀ (50% Tissue Culture Infective Dose) or Inf.U. (Infectious Unit). The rMVA virus may be administered in a volume ranging between 0.25 and 1.0 ml. Compositions may be administered two or more times, weeks or months after administration of the priming composition, for example, about 1 or 2 weeks or 3 weeks, or 4 weeks, or 6 weeks, or 8 weeks, or 12 weeks, or 16 weeks, or 20 weeks, or 24 weeks, or 28 weeks, or 32 weeks or one to two years after the first administration of the first composition. Additional administrations of the compositions may be administered 6 weeks to 5 years after the boosting step (b), such as 6, 7, 8, 9, 10, 11, 12, 13, 14, 15, 16, 17, 18, 19, 20, 21, 22, 23, 24 or 25 weeks, or 7, 8, 9, 10, 11 or 12 months, or 2, 3, 4 or 5 years, after the initial boosting inoculation. Optionally, the further administration step (c) can be repeated one or more times as needed.

Combination therapies

The vaccines and compositions of the disclosure may be used in combination with at least one additional cancer therapeutic agent for treating multiple myeloma.

The additional cancer therapeutic agent may be a chemotherapeutic agent, an immunomodulatory agent, a corticosteroid, a radiation therapy, a targeted therapy, a high dose chemotherapy (HDC) and stem cell transplant (SCT), a checkpoint inhibitor, an antibiotic, an immunostimulating agent, or cellular therapy, or a surgery, or any combination thereof.

Exemplary chemotherapeutic agents are proteasome inhibitors; alkylating agents; microtubule inhibitors; nitrosoureas; antineoplastic antimetabolites; antitumor antibiotics; plant alkyloids; taxanes; hormonal agents; and miscellaneous agents, such as busulfan, a platin compound (e.g., carboplatin, cisplatin), chlorambucil, cyclophosphamide, dacarbazine, ifosfamide, mechlorethamine hydrochloride, melphalan, procarbazine, thiotepa, uracil mustard, 5-fluorouracil, 6-mercaptopurine, capecitabine, cytosine arabinoside, floxuridine, fludarabine, gemcitabine, methotrexate, thioguanine, dactinomycin, daunorubicin, doxorubicin, idarubicin, mitomycin-C, and mitoxantrone, vinblastine, vincristine, vindesine, vinorelbine, paclitaxel, and docetaxel.

In some embodiments, the chemotherapeutic agent is a proteasome inhibitor.

In some embodiments, the proteasome inhibitor is bortezomib, carfilzomib, marizomib or ixazomib, or any combination thereof.

In some embodiments, the chemotherapeutic agent is an alkylating agent.

“Alkylating agent” refers to family of DNA alkylating agents including cyclophosphamide, ifosfamide, melphalan or nitrosoureas. Cyclophosphamide is marketed under the trade name Cyclostin™. Ifosfamide is marketed under the trade name Holoxan™. Melphalan is marketed under the trade name ALKERAN®. Nitrosureas include carmustine, lomustine and semustine. Carmustine is marketed under the trade name BiCNU®. Lomustine is marketed under the trade name GLEOSTINE®.

In some embodiments, the alkylating agent is melphalan, cyclophosphamide, ifosfamide or nitrosourea, or any combination thereof.

In some embodiments, the chemotherapeutic agent is a microtubule inhibitor.

“Microtubule inhibitor” (MTI) refers to microtubule destabilizing compounds and microtubule polymerization inhibitors including to taxanes, such as paclitaxel and docetaxel, vinca alkaloids, such as vinblastine or vinblastine sulfate, vincristine or vincristine sulfate and vinorelbine. Paclitaxel is marketed under the trade name TAXOL®. Docetaxel is marketed under the trade name TAXOTERE®. Vinblastine sulfate is marketed under the trade name Vinblastin R.P™. Vincristine sulfate is marketed under the trade name Farmistin™. Vinorelbine is marketed under the trade name NAVELBINE®.

In some embodiments, the microtubule inhibitor is a taxane or a vinca alkaloid, or any combination thereof.

In some embodiments, the vinca alkaloid is vincristine.

In some embodiments, the chemotherapeutic agent an antineoplastic antimetabolite.

“Antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name XELODA®. Gemcitabine is marketed under the trade name GEMZAR®

Exemplary immunomodulatory agents include glutamic acid derivatives,

“Glutamic acid derivative” refers to immunomodulatory drugs that are derivatives of glutamic acid such as lenalidomide, thalidomide and pomalidomide. Lenalinomide is marketed under the trade name REVLIMID®. Thalidomide is marketed under the trade name THALOMID®. Pomalidomide is marketed under the trade name POMALYST®

In some embodiments, the glutamic acid derivative is lenalinomide, pomalidomide or thalidomide, or any combination thereof.

Exemplary corticosteroids include dexamethasone or prednisone, or any combination thereof.

Radiation therapy may be administered using various methods, including external-beam therapy, internal radiation therapy, implant radiation, stereotactic radiosurgery, systemic radiation therapy, radiotherapy and permanent or temporary interstitial brachytherapy. External-beam therapy involves three-dimensional, conformal radiation therapy where the field of radiation is designed, local radiation (e.g., radiation directed to a preselected target or organ), or focused radiation. Focused radiation may be selected from stereotactic radiosurgery, fractionated stereotactic radiosurgery or intensity-modulated radiation therapy. Focused radiation may have particle beam (proton), cobalt-60 (photon) linear accelerator (x-ray) as a radiation source (see e.g. WO 2012/177624). “Brachytherapy,” refers to radiation therapy delivered by a spatially confined radioactive material inserted into the body at or near a tumor or other proliferative tissue disease site, and includes exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner include both solids and liquids. The radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material may also be a fluid made from any solution of radionuclide(s), e.g., a solution of I-125 or I-131, or a radioactive fluid can be produced using a slurry of a suitable fluid containing small particles of solid radionuclides, such as Au-198, Y-90. The radionuclide(s) may be embodied in a gel or radioactive micro spheres.

Targeted therapies include, but are not limited to, anti-CD38 antibodies (e.g., daratumumab and elotuzumab), anti-BCMA antibodies or CAR-Ts, anti-GPRCSD antibodies or CAR-Ts, and anti-SLAMF7 antibodies (e.g., elotuzumab).

Stem cell transplant (SCT) may be an autologous SCT (ASCT), allogenic SCT or syngeneic SCT. In some embodiments, SCT is ASCT.

In some embodiments, the additional therapeutics comprise bortezomib and dexamethasone.

In some embodiments, bortezomib is administered at a dose of about 1.3 mg/m² and dexamethasone is administered at a dose of about 20 mg.

In some embodiments, the additional therapeutics comprise lenalidomide and dexamethasone.

In some embodiments, lenalidomide is administered at a dose of about 25 mg and dexamethasone is administered at a dose of between about 20 mg and about 40 mg.

In some embodiments, the additional therapeutics comprise pomalidomide and dexamethasone.

In some embodiments, pomalidomide is administered at a dose of about 25 mg and dexamethasone is administered at a dose of between about 20 mg and about 40 mg.

In some embodiments, the additional therapeutics comprise bortezomib, melphalan and prednisone.

In some embodiments, bortezomib is administered at a dose of about 1.3 mg/m², melphalan is administered at a dose of about 9 mg/m² and prednisone is administered at a dose of about 60 mg/m².

In some embodiments, the additional therapeutics comprise bortezomib, thalidomide and dexamethasone.

In some embodiments, bortezomib is administered at a dose of about 1.3 mg/m², thalidomide is administered at a dose of about 25 mg and dexamethasone is administered at a dose of about between about 20 mg and about 40 mg.

In some embodiments, the subject is eligible for high dose chemotherapy (HDC) and stem cell transplant (SCT).

“High dose chemotherapy” (HDC) and “autologous stem cell transplant” (ASCT) refer to the treatment of subjects with newly diagnosed multiple myeloma who are considered fit (e.g. subjects are “eligible”). Subjects under the age of 65 years who have one or more comorbidities likely to have a negative impact on tolerability of HDC and ASCT or subjects over the age of 65 years are usually not considered eligible for HDC and ASCT due to their frail physical status which increases the risk of mortality and transplant-related complications (e.g. subjects are “ineligible”). An exemplary comorbidity is a renal dysfunction. Exemplary HDC regimens are melphalan at a dose of 200 mg/m² body surface areawith dose reductions based on age and renal function, cyclophosphamide and melphalan, carmustine, etoposide, cytarabine, and melphalan (BEAM), high-dose idarubicin, cyclophosphamide, thiotepa, busulfan, and cyclophosphamide, busulfan and melphalan, and high-dose lenalidomide (Mahajan et al., Ther Adv Hematol 9:123-133, 2018). Cyclophosphamide is marketed under the trade name Cyclostin™. Melphalan is marketed under the trade name ALKERAN®. Carmustine is marketed under the trade name BiCNU®. Etoposide is marketed under the trade name VEPESID®. Cytarabine is marketed under the trade name CYTOSAR-U®. Idarubicin is marketed under the trade name IDAMYCN®. Thitepa is marketed under the trade name THIOPLEX®. Lenalidomide is marketed under the trade name REVLIMID®.

In some embodiments, SCT is autologous SCT (ASCT), allogenic SCT or syngeneic SCT.

In some embodiments, SCT is ASCT.

In some embodiments, HDC is melphalan.

Exemplary checkpoint inhibitors are antagonists of PD-1, PD-L1, PD-L2, VISTA, BTNL2, B7-H3, B7-H4, HVEM, HHLA2, CTLA-4, LAG-3, TIM-3, BTLA, CD160, CEACAM-1, LAIR1, TGFβ, IL-10, Siglec family protein, KIR, CD96, TIGIT, NKG2A, CD112, CD47, SIRPA or CD244. “Antagonist” refers to a molecule that, when bound to a cellular protein, suppresses at least one reaction or activity that is induced by a natural ligand of the protein. A molecule is an antagonist when the at least one reaction or activity is suppressed by at least about 30%, 40%, 45%, 50%, 55%, 60%, 65%, 70%, 75%, 80%, 85%, 90%, 95%, or 100% more than the at least one reaction or activity suppressed in the absence of the antagonist (e.g., negative control), or when the suppression is statistically significant when compared to the suppression in the absence of the antagonist. Antagonist may be an antibody, a soluble ligand, a small molecule, a DNA or RNA such as siRNA. Exemplary antagonists of checkpoint inhibitors are described in U.S. Pat. Publ. No. 2017/0121409.

In some embodiments, one or more vaccines or compositions of the disclosure is administered in combination with a CTLA-4 antibody, a CTLA4 ligand, a PD-1 axis inhibitor, a PD-L1axis inhibitor, a TLR agonist, a CD40 agonist, an OX40 agonist, hydroxyurea, ruxolitinib, fedratinib, a 41BB agonist, aa CD28 agonist, a STING agonist, a RIG-I agonist, TCR-T therapy, CAR-T therapy, FLT3 ligand, aluminum sulfate, BTK inhibitor, CD38 antibody, CDK inhibitor, CD33 antibody, CD37 antibody, CD25 antibody, GM-CSF inhibitor, IL-2, IL-15, IL-7, CD3 redirection molecules, pomalimib, IFNγ, IFNα, TNFα, VEGF antibody, CD70 antibody, CD27 antibody, BCMA antibody or GPRC5D antibody, or any combination thereof.

In some embodiments, the checkpoint inhibitor is ipilimumab, cetrelimab, pembrolizumab, nivolumab, sintilimab, cemiplimab, toripalimab, camrelizumab, tislelizumab, dostralimab, spartalizumab, prolgolimab, AK-105, HLX-10, balstilimab, MEDI-0680, HX-008, GLS-010, BI-754091, genolimzumab, AK-104, MGA-012, F-520, 609A, LY-3434172, AMG-404, SL-279252, SCT-I10A, RO-7121661, ICTCAR-014, MEDI-5752, CS-1003, XmAb-23104, Sym-021, LZM-009, hAB21, BAT-1306, MGD-019, JTX-4014, budigalimab, XmAb-20717, AK-103, MGD-013, IBI-318, sasanlimab, CC-90006, avelumab, atezolizumab, durvalumab, CS-1001, bintrafusp alpha, envafolimab, CX-072, GEN-1046, GS-4224, KL-A167, BGB-A333, SHR-1316, CBT-502, IL-103, KN-046, ZKAB-001, CA-170, TG 1501, LP-002, INCB-86550, ADG-104, SHR-1701, BCD-135, IMC-001, MSB-2311, FPT-155, FAZ-053, HLX-20, iodapolimab, FS-118, BMS-986189, AK-106, MCLA-145, IBI-318 or CK-301, or any combination thereof.

In some embodiments, one or more vaccines or compositions of the disclosure are administered in combination with ipilimumab, cetrelimab, pembrolizumab, nivolumab, sintilimab, cemiplimab, toripalimab, camrelizumab, tislelizumab, dostralimab, spartalizumab, prolgolimab, balstilimab, budigalimab, sasanlimab, avelumab, atezolizumab, durvalumab, envafolimab or iodapolimab, or any combination thereof.

In some embodiments, one or more vaccines or compositions of the disclosure is administered in combination with a CTLA-4 antibody, a CTLA4 ligand, a PD-1 axis inhibitor, a PD-L1 axis inhibitor, a TLR agonist, a CD40 agonist, an OX40 agonist, hydroxyurea, ruxolitinib, fedratinib, a 41BB agonist, aa CD28 agonist, a STING agonist, a RIG-I agonist, TCR-T therapy, CAR-T therapy, FLT3 ligand, aluminum sulfate, BTK inhibitor, CD38 antibody, CDK inhibitor, CD33 antibody, CD37 antibody, CD25 antibody, GM-CSF inhibitor, IL-2, IL-15, IL-7, CD3 redirection molecules, pomalimib, IFNγ, IFNα, TNFα, VEGF antibody, CD70 antibody, CD27 antibody, BCMA antibody or GPRCSD antibody, or any combination thereof.

In some embodiments, the second therapeutic agent may be administered in combination with a first composition of the first administration or a second composition of the second administration or a third composition of the third administration, or a fourth composition of the fourth administration.

In some embodiments, the anti-CTLA-4 antibody is combined with any of the first, or the second, or the third, or the fourth administration of the composition of the disclosure.

In some embodiments, the anti-PD-1 or anti-PD-L1 antibody is combined with any of the first, or the second, or the third, or the fourth administration of the composition of the disclosure.

In some embodiments, the checkpoint inhibitors are administered at as dose of about 0.5 to about 5 mg/kg, about 5 to about 10 mg/kg, about 10 to about 15 mg/kg, about 15 to about 20 mg/kg, about 20 to about 25 mg/kg, about 20 to about 50 mg/kg, about 25 to about 50 mg/kg, about 50 to about 75 mg/kg, about 50 to about 100 mg/kg, about 75 to about 100 mg/kg, about 100 to about 125 mg/kg, about 125 to about 150 mg/kg, about 150 to about 175 mg/kg, about 175 to about 200 mg/kg, about 200 to about 225 mg/kg, about 225 to about 250 mg/kg, or about 250 to about 300 mg/kg.

### EXAMPLES

The following examples are provided to further describe some of the embodiments disclosed herein. The examples are intended to illustrate, not to limit, the disclosed embodiments.

### Example 1: Identification of Neoantigens by Bioinformatics

A computational framework was developed to analyze various multiple myeloma RNA-seq datasets by bioinformatics means to identify common multiple myeloma neoantigens resulting from gene fusion events that resulted in generation of novel peptide sequences, intron retention, alternatively spliced variants, aberrant expression of developmentally silenced genes or point mutations.

The datasets queried were:
The Genotype-Tissue Expression (GTEx) Consortium. This dataset encompasses 6137 RNA-seq datasets from 49 normal tissues and was used to assess frequency of multiple myeloma neoantigen candidates in normal tissue.
Immune cell-type specific RNA-seq dataset. This internal study comprised of 110 RNA-seq datasets obtained from 20 immune cell-types (T cells, B cells. NK cells and Myeloid cell-types) derived from five healthy donors.
MMRF CoMMpass study (https://themmrf.org/we-are-curing-multiple-myeloma/mmrf-commpass-study/). This study encompasses 807 RNA-seq datasets obtained from newly diagnosed multiple myeloma patients.
SMM 2001 study. This study comprised of 130 RNA-seq datasets obtained from patients with smoldering multiple myeloma.
MMY3007 study. This study encompasses 586 RNA-seq datasets obtained from patients enrolled in Daratumumab clinical trial.
MMY3003-3004 study. This study encompasses 861 RNA-seq datasets obtained from patients enrolled in Daratumumab clinical trial.

All samples included in the RNA-seq database (MMRF and clinical trial studies described. above) are CD138+ cells enriched from patient bone marrow aspirates.

Quality control (QC) of raw data was conducted prior to analysis. Sequencing reads were first trimmed to remove Illumina's adapter sequences and reads mapping to human tRNA and rRNA were removed from downstream analysis. Reads were also trimmed of low-quality base calls (<10 Phred quality score; indicating a base with a 1 in 10 probablility of being incorrect) at either ends.

Trimmed reads with less than 25 base pairs (bp) were removed from the datasets. Additionally, following QC steps were considered to remove poor quality reads: remove reads having maximal base quality score less than 15, remove reads with average base quality score less than 10, remove reads having polyATCG rate >80%, remove RNA sequences in which one of the two reads failed.

Reads were later mapped to Human Genoese Build 38 using ArrayStudio ((https_//www_omicsoft_com/array-studio/) platform. NCBI's Refseq gene model (release date June 6, 2017) was used to map reads to known exonic regions of human genome.

### Identification of Gene Fusion Events

FusionMap algorithm (Ge H et al., Bioinformatics. 2011 Jul. 15; 27(14):1922-8.) was used to identify gene fusion events in the cancer datasets described above. FusionMap detected fusion junctions based on reads that contained the fusion position in the middle region of the sequencing reads. This was followed by searching possible fusion junction positions from the consensus of seed reads. FusionMap built the reference index based on the pseudo fusion library and aligned unmapped potential fusion reads to this pseudo reference. Reads mapped during this step were considered as rescue reads.

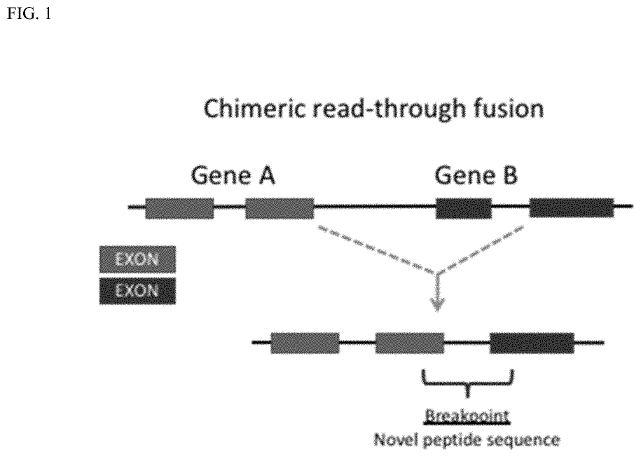

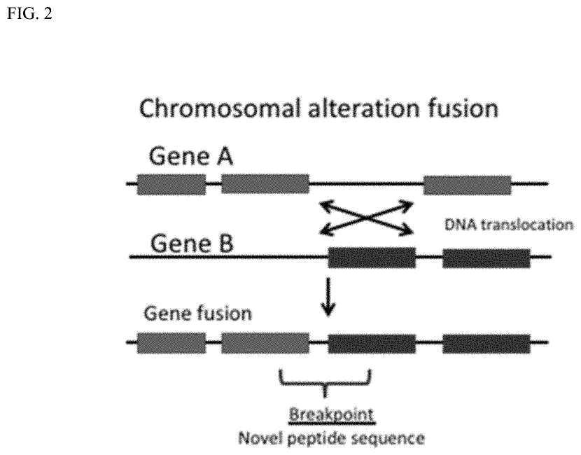

This algorithm identified both chimeric read-through fusions as shown in FIG. 1 and gene fusion events resulting from chromosomal translocations as shown in FIG. 2. A gene fusion event was called in a RNA-seq dataset when following criteria were met: at least two seed reads with different mapping position in the genome, at least four seed and rescued reads supporting the fusion junction and at least one junction spanning read pair. Gene fusion events coming from gene pairs that shared high sequence similarity (orthologs and protein families) were ignored from downstream analysis.

Shared neoantigens originating from gene fusion events were identified using following criteria: the incidence of gene fusion event in a disease cohort was to be greater than 5%, the occurrence of the gene fusion event was to be less than 1% in the entire GTEx dataset using a lenient criteria (at least 2 seed reads and one junction spanning read) and the occurrence of the gene fusion event was to be <=2 RNA-seq datasets derived from normal immune cell-types. The open reading frame from Gene A (FIG. 1 and FIG. 2) was used to obtain protein sequence originating from the identified novel junction.

### Identification of Splice Variants

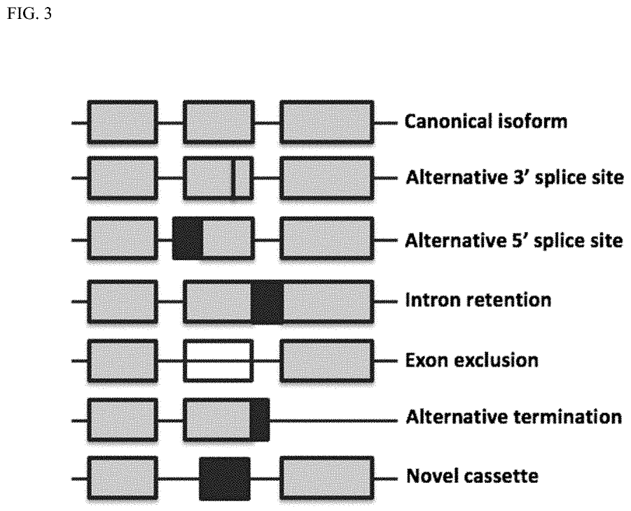

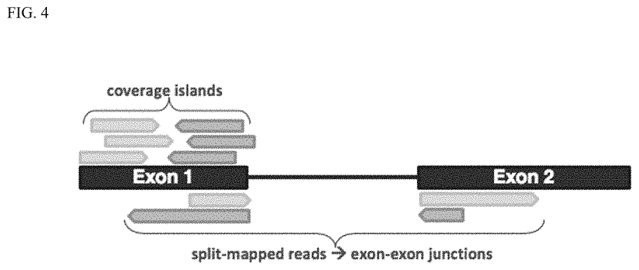

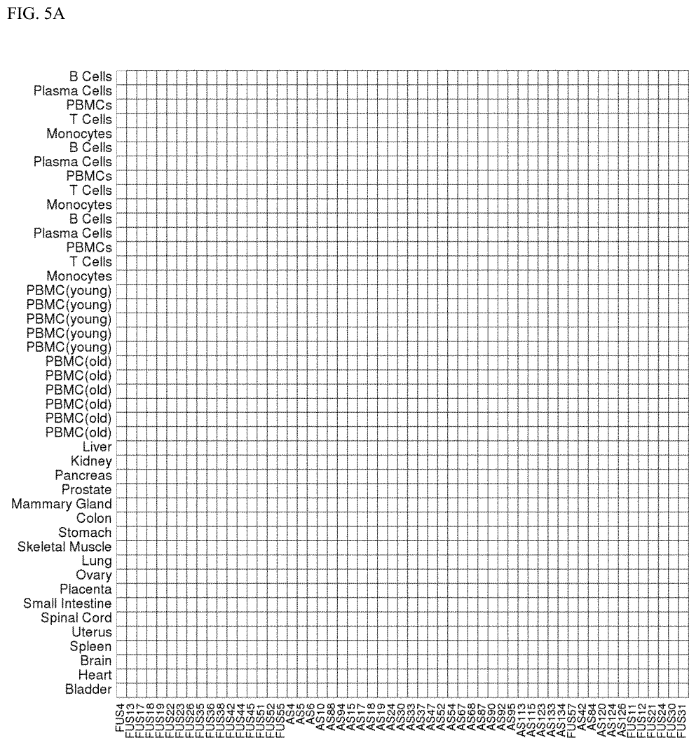

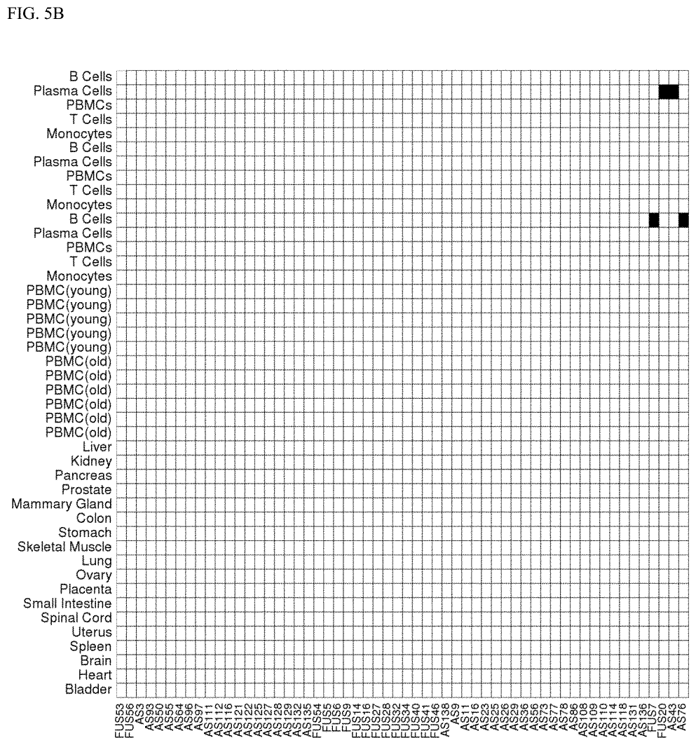

A custom bioinformatics process was developed to analyze paired-end RNA-seq data to identify potential neoantigens arising from alternative splicing events. Utilizing the developed process, splice variants with alternative 5′ or 3′ splice sites, retained introns, excluded exons, alternative terminations or insertion(s) of novel cassettes as shown in FIG. 3 were identified. The process identified splice variants that were not present in the NCBI's RefSeq gene model through two main functionalities: 1) Identification of novel junctions based on sequencing reads with alignment gaps >5 base pair and ≥15 base pair aligned on each side of the gap, henceforth referred to as split-mapped reads. For each RNA-seq dataset, novel junctions were called if they were supported by at least 5 split-mapped reads and one mate pair of junction-spanning reads 2) Identification of islands of aligned reads, henceforth referred to as coverage islands. FIG. 4 shows the cartoon of the approach.

In order to assess the signal to noise ratio in each sample, where genomic DNA and pre-mRNA are potential contributors to noise, two parameters were computed from a set of 200 highly expressed housekeeping genes:
1. Intron depth of coverage (IDC): 90^{th} percentile depth of coverage for all housekeeping intronic bases. If the coverage of a particular region fell below this value, the first base where this occurred was defined as a coverage island boundary.
2. Intron/exon coverage ratio (IECR): 90^{th} percentile of the ratio between median intron coverage and median coverage of the nearest upstream exon of all housekeeping gene introns

Following criteria was used to classify the various splice variants:

### Alternative 3′/5′ Splice Site Identification:

Novel splice site boundary was defined by split-mapped reads
Intronic region resulting from using the splice site (if applicable) exceeded IECR and entire region exceeded IDC

### Novel Cassette Identification:

Two novel splice sites in an intronic region defined by split-mapped reads
Region between the two splice sites exceeded IECR and entire region exceeded IDC

### Intron Retention Identification:

Intronic region exceeded IECR and entire region exceeded IDC
At least 5 reads spanned both intron-exon boundaries, with at least 15 hp aligned on each side of the boundaries

### Alternative Termination Identification:

3′ boundary defined as the edge of a coverage island that did not fall within 60 bp of the 3′ end of a canonical exon
Any intronic regions between 5′ end of a canonical exon and the 3′ boundary exceeded IECR and entire region exceeded IDC

### Exon Exclusion Identification:

Novel junction defined by split-mapped reads where one or more canonical exons were skipped

Shared neoantigens originating from aberrant splicing events were identified using following criteria: the incidence of a splicing event in a disease cohort was to be greater than 5%, the occurrence of the splicing event was to be less than 1% in the entire GTEx dataset using a lenient criteria (at least 2 split-mapped reads) and the splicing event was to be present in ≤2 RNA-seq datasets derived from normal immune cell-types. For exon exclusion, novel cassette, and alternative 3′/5′ splice sites, events were to have a median split-mapped read counts per million mapped reads (CPM)≥0.05 and a median percent spliced-in (PSI) >0.1, calculated using the formula below:

$$PSI = \frac{inclusion reads}{inclusion + exclusion reads}$$

Events with median value of 0.05≥PSI≥0.1 were selected if the aberrantly spliced gene was found to be 2-fold upregulated in disease cohort versus healthy tissue differential gene expression analysis. For alternative termination and retained introns, events were to have a median number of split-mapped CPM≥0.1 and a median PSI≥0.5. They were also to be detected in ≥1% of at least two disease cohorts.

### Isoform Prediction and Translation:

In order to assemble isoforms containing the alternatively spliced neoantigens, canonical exons neighboring the novel spliced features were identified using the split-mapped reads. The most highly expressed isoform that could potentially contain the predicted neoepitope was chosen for translation into the corresponding protein by choice of the appropriate open reading frame. The neoantigen portion of the protein sequence was extracted and concatenated with an additional 8 amino acid residues upstream of the first altered amino acid. This protein sequence was then used for subsequent validation studies.

### Identification of DNA Mutation Neoantigens

Datasets generated by the Broad Institute containing exome sequencing data from patients with Multiple Myeloma (Lohr, J. G. et. al Cancer Cell 2014, Jan. 13; 25(1) 91-101) were examined. Mutation calls published by the consortia that generated this dataset were downloaded, and gene mutations that were present in >5% of the patient population or in genes known to be critical drivers of cancer were identified. For these genes, the most recurrent point mutations were chosen for further study, and genes that did not have recurrent point mutations at a position were dropped from consideration. For each single point mutation chosen, a 17 mer peptide with the mutated amino acid at its center was identified for further validation studies

### Splicing Isoform Prediction

In certain cases, there were multiple reading frames and exons upstream of identified splicing events that could impact canonical peptide sequence preceding a neoepitope sequence. In these genes, it was determined which canonical exons neighbored each neoepitope feature based on split-mapped reads present at the exon boundaries. A most highly expressed isoform with highest average expression in a disease cohort with highest prevalence of an event that could contain the predicted neoepitope was chosen for translation into a corresponding protein by choice of an open reading frame associated with the isoform. A neoepitope portion of the protein sequence was extracted, with an additional 8 amino acid residues upstream of the first altered amino acid included and used for subsequent validation studies. A similar procedure was followed to identify putative immunogenic antigens from DNA frameshift alterations. For both frameshift deletions and insertions, the resulting DNA sequence was translated into the corresponding protein by choice of appropriate open reading frame, and a frameshift altered portion of the protein sequence was extracted, with an additional 8 amino acid residues upstream of the first altered amino acid included.

Table 5 shows gene origin and amino acid sequence of identified neoantigens that arose from gene fusion (FUS) events. In Table 5, bolded letters indicate canonical amino acids from gene 1. Italic letters indicate canonical amino acids from gene 2 for in-frame gene fusion events. Unbolded letters indicate novel amino acid sequences generated dfrom out-of-frame gene fusion events. Table 6 shows the full names of the fusion genes. Table 7 shows their corresponding polynucleotide sequences.

TABLE 5

| Neoantigen | | | SEQ ID |
|---|---|---|---|
| ID | Fusion Gene | Amino acid sequence | NO: |
| FUS1 | MCL1-> ADAMTSL4-AS1 | ISNKIALLQTF | 1 |
| FUS2 | MCL1-> ADAMTSL4-AS1 | WLVKQRGWANLLEKSAEQICT | 3 |
| | | GEFKAKASQS | |
| FUS3 | MCL1-> ADAMTSL4-AS1 | NHETAFQGKPFREVS | 5 |
| FUS4 | CD5L-> FCRL1 | DVAVICSGRRSARDP | 7 |
| FUSS | CD5L-> FCRL1 | DVAVICSGVPVADVS | 9 |
| FUS6 | CD5L-> FCRL1 | EDVAVICSELFLIASP | 11 |
| FUS7 | FAM98A- | GRAGQGGGMISVSQEFIK | 13 |
| | > LOC105374454 | | |
| FUS8 | ANKRD44-> ANKRD44- | AVLKLTDQHNAVKMGEERCVS | 15 |
| | IT1 | SEI | |
| FUS9 | EAF2-> SLC15A2 | SGLLMNTLTVLILYFLY | 17 |
| FUS10 | DDX21-> JCHAIN | APKPKKMKNHLLFW | 19 |
| FUS11 | GAB1-> SMARCA5 | GKSTPPRKEIFDDASP | 21 |
| FUS12 | NUDT12-> LINC02115 | ARWFTREQGSEN | 23 |
| FUS13 | KCNQ5-> KCNQ5-IT1 | AFIYHAFVGSTKTAWRTTL | 25 |
| FUS14 | UBE2J1-> GABRR2 | LARQISFKSLI | 27 |
| FUS15 | UBE2J1-> GABRR2 | QPRDNHTDQVTYIRRTLM | 29 |
| FUS16 | UBE2J1-> GABRR2 | PRDNHTDHENPRGSDGQGRWK | 31 |
| | | CPSQVTYIRRTLM | |
| FUS17 | HBS1L-> ALDH8A1 | AAGVVTEIEAAVKA | 33 |
| FUS18 | CADPS2-> RNF148 | NAVRSYYEGTENIVAV | 35 |
| FUS19 | CADPS2-> RNF148 | NAVRSYYETNGGMSFLRITP | 37 |
| FUS20 | PTGES2-> SLC25A25-AS1 | ISKRLKSRRRSGWQLNRAGNR | 39 |
| | | GLSPGLGLFPRGCCRWGGAYTR | |
| | | LPSANQT | |
| FUS21 | FOSB-> KLF6 | PPTAAASQTCLELER | 41 |
| FUS22 | IGLL5-> COMMD3- | ACGACGAGFFIKQKCIEQRESRS | 43 |
| | BMI1, BMI1 | LS | |
| FUS23 | NDUFB8-> SEC31B | VYPVYQPVVSQAGLGELWQWA | 45 |
| | | SGKLRGYCWRRGQWHAYSIQC | |
| | | DPHPVFGEGACDCSETEAHGGC | |
| | | QSPRLESFPGQPPGFRGQRF | |
| FUS24 | B2M-> DUSP5 | SGLEAIQRGYETFYSE | 47 |
| FUS25 | CTSD-> IFITM10 | SPEDYTLKAQGPGQCP | 49 |
| FUS26 | P2RY6-> ARHGEF17 | ARHLLTLGTCGSTWP | 51 |
| FUS27 | TBCEL-> TECTA | PQEEVPFRSITTELFPSMC | 53 |
| FUS28 | NCOR2-> UBC | QIIYDENRTMQIFVKT | 55 |
| FUS29 | TPM4-> UBC | GERERREKTMQIFVKT | 57 |
| FUS30 | ZFP36-> UBC | MDLTAIYETMQIFVKT | 59 |
| FUS31 | FOSB-> UBC | PPTAAASQTMQIFVKT | 61 |
| FUS32 | GANC-> CAPN3 | SSVTTHSSGNLRESPIYH | 63 |
| FUS33 | B2M-> KLF2 | SGLEAIQREKPYHCNW | 65 |
| FUS34 | CLN6-> CALML4 | VAPSGLYYCG | 67 |
| FUS35 | TMED3-> KIAA1024 | NRVTALTQN | 69 |
| FUS36 | TNFRSF17-> SNX29 | IEKSISARITEQ | 71 |
| FUS37 | NLRP1-> LOC728392 | PSDCSIRKTLLSLPGGVETGTRR | 73 |
| | | SCLTRRPRPGSVRDSRDLWLQTP | |
| | | PARRPRAGALLRMRLWGP | |
| FUS38 | CD79B-> GH1 | GEVKWSVGSRTSLLL | 75 |
| FUS39 | TPM4-> KLF2 | LTEKKASDVRSPTTATGTAAAG | 77 |
| | | SLRAQTSSRATTESTRATGHSSA | |
| | | ICAIVPSRAPITWRCT | |
| FUS40 | KLF2-> TPM4 | CRERGLQEAEGDVAAL | 79 |
| FUS41 | FOSB-> KLF2 | PPTAAASQVRSPTTATGTAAAG | 81 |
| | | SLRAQTSSRATTESTRATGHS | SA |
| | | ICAIVPSRAPITWRCT | |
| FUS42 | RBM42-> ETV2 | GGPPFVGPLRGPRAQPSLSGLC | 83 |
| | | GRRTGSRDTIKIPGQT | |
| FUS43 | STX16-> STX16- | GLKQLHKAGYEEETQNNRSGL | 85 |
| | NPEPL1, NPEPL1, NPEPL | WNYMELWQAAL | |
| | 1 | | |
| FUS44 | ZGPAT-> LIME1 | LDQCVETLCSLSKSD | 87 |
| FUS45 | IFNAR2-> IL10RB | LLPPGQESAIIGPPGMQ | 89 |
| FUS46 | GCNA-> LOC101059915 | YECTGCKTSFPHRSQS | 91 |
| FUS47 | CCAR1-> SNORD98 | QHPARLVKCGTQ | 93 |
| FUS48 | NDUFA11-> FUT5 | GLTLGARTTLTHGSPGPSQATV | 95 |
| | | AVAPLSGRAAVSAAGGCVFLLL | |
| | | PACVPRRCHWIP | |
| FUS49 | ZFP36-> PLEKHG2 | MDLTAIYECCNLSVSE | 97 |
| FUS50 | ZNF772-> VN1R1 | VIVDPIQGYLRQWQLLD | 99 |
| FUS51 | POU2AF1-> COLCA1 | PPLITNVTEKPWKHTRIEMPLA | 101 |
| | | RLTRP | |
| FUS52 | SELPLG-> TMEM119 | MEPTTKRGPGGTMVSAAAPS | 103 |
| FUS53 | PTPRG-> C3orf14 | RPGVFTDIL | 105 |
| FUS54 | ZFP36-> PLEKHG2 | MDLTAIYEDSAGPLSHA | 107 |
| FUS55 | FAM214A-> ARPP19 | LRYLIHLRKWKIK | 109 |
| FUS56 | ZNF772-> VN1R1 | FALMASLGIPQTMAA | 111 |
| FUS57 | MED12-> NLGN3 | RQLQQQLSSLSGPEGVPFLKLW | 113 |
| | | CLDDLLSTLLGTCRCPSRAQF | |

TABLE 6

| Neoantigen | | |
|---|---|---|
| ID | Full Name of Fusion Gene 1 | Full Name(s) of Fusion Gene 2 |
| FUS1 | BCL2 family apoptosis regulator | ADAMTSL4 antisense RNA 1 |
| FUS2 | BCL2 family apoptosis regulator | ADAMTSL4 antisense RNA 1 |
| FUS3 | BCL2 family apoptosis regulator | ADAMTSL4 antisense RNA 1 |
| FUS4 | CD5 molecule like | Fc receptor like 1 |
| FUS5 | CD5 molecule like | Fc receptor like 1 |
| FUS6 | CD5 molecule like | Fc receptor like 1 |
| FUS7 | family with sequence similarity 98 | uncharacterized LOC105374454 |
| | member A | |
| FUS8 | ankyrin repeat domain 44 | ANKRD44 intronic transcript 1 |
| FUS9 | ELL associated factor 2 | solute carrier family 15 member 2 |
| FUS10 | DExD-box helicase 21 | joining chain of multimeric IgA and |
| | | IgM |
| FUS11 | GRB2 associated binding protein 1 | SWI/SNF related, matrix associated, |
| | | actin dependent regulator of |
| | | chromatin, subfamily a, member 5 |
| FUS12 | nudix hydrolase 12 | long intergenic non-protein coding |
| | | RNA 2115 |
| FUS13 | potassium voltage-gated channel | KCNQ5 intronic transcript 1 |
| | subfamily Q member 5 | |
| FUS14 | ubiquitin conjugating enzyme E2 J1 | gamma-aminobutyric acid type A |
| | | receptor rho2 subunit |
| FUS15 | ubiquitin conjugating enzyme E2 J1 | gamma-aminobutyric acid type A |
| | | receptor rho2 subunit |
| FUS16 | ubiquitin conjugating enzyme E2 J1 | gamma-aminobutyric acid type A |
| | | receptor rho2 subunit |
| FUS17 | HBS1 like translational GTPase | aldehyde dehydrogenase 8 family |
| | | member A1 |
| FUS18 | calcium dependent secretion activator 2 | ring finger protein 148 |
| FUS19 | calcium dependent secretion activator 2 | ring finger protein 148 |
| FUS20 | prostaglandin E synthase 2 | SLC25A25 antisense RNA 1 |
| FUS21 | FosB proto-oncogene, AP-1 | Kruppel like factor 6 |
| | transcription factor subunit | |
| FUS22 | immunoglobulin lambda like | COMMD3-BMI1 readthrough, |
| | polypeptide 5 | polycomb complex protein BMI-1 |
| FUS23 | NADH:ubiquinone oxidoreductase | SEC31 homolog B, COPII coat |
| | subunit B8 | complex component |
| FUS24 | beta-2-microglobulin | dual specificity phosphatase 5 |
| FUS25 | cathepsin D | interferon induced transmembrane |
| | | protein 10 |
| FUS26 | pyrimidinergic receptor P2Y6 | Rho guanine nucleotide exchange |
| | | factor 17 |
| FUS27 | tubulin folding cofactor E like | tectorin alpha |
| FUS28 | nuclear receptor corepressor 2 | ubiquitin C |
| FUS29 | tropomyosin 4 | ubiquitin C |
| FUS30 | ZFP36 ring finger protein | ubiquitin C |
| FUS31 | FosB proto-oncogene, AP-1 | ubiquitin C |
| | transcription factor subunit | |
| FUS32 | glucosidase alpha, neutral C | calpain 3 |
| FUS33 | beta-2-microglobulin | Kruppel like factor 2 |
| FUS34 | ceroid-lipofuscinosis, neuronal 6, late | calmodulin like 4 |
| | infantile, variant | |
| FUS35 | transmembrane p24 trafficking protein | Major intrinsically disordered |
| | 3 | Notch2-binding receptor 1 |
| FUS36 | TNF receptor superfamily member 17 | sorting nexin 29 |
| FUS37 | NLR family pyrin domain containing 1 | uncharacterized LOC728392 |
| FUS38 | CD79b molecule | growth hormone 1 |
| FUS39 | tropomyosin 4 | Kruppel like factor 2 |
| FUS40 | Kruppel like factor 2 | tropomyosin 4 |
| FUS41 | FosB proto-oncogene, AP-1 | Kruppel like factor 2 |
| | transcription factor subunit | |
| FUS42 | RNA binding motif protein 42 | ETS variant 2 |
| FUS43 | syntaxin 16 | STX16-NPEPL1 readthrough, |
| | | aminopeptidase-like 1 |
| FUS44 | zinc finger CCCH-type and G-patch | Lck interacting transmembrane |
| | domain containing | adaptor 1 |
| FUS45 | interferon alpha and beta receptor | interleukin 10 receptor subunit beta |
| | subunit 2 | |
| FUS46 | Germ cell nuclear antigen | chromosome X open reading frame |
| | | 49-like |
| FUS47 | cell division cycle and apoptosis | small nucleolar RNA, C/D box 98 |
| | regulator 1 | |
| FUS48 | NADH:ubiquinone oxidoreductase | fucosyltransferase 5 |
| | subunit A11 | |
| FUS49 | ZFP36 ring finger protein | pleckstrin homology and RhoGEF |
| | | domain containing G2 |
| FUS50 | zinc finger protein 772 | vomeronasal 1 receptor 1 |
| FUS51 | POU class 2 associating factor 1 | colorectal cancer associated 1 |
| FUS52 | selectin P ligand | transmembrane protein 119 |
| FUS53 | protein tyrosine phosphatase, receptor | chromosome 3 open reading frame 14 |
| | type G | |
| FUS54 | ZFP36 ring finger protein | pleckstrin homology and RhoGEF |
| | | domain containing G2 |
| FUS55 | family with sequence similarity 214 | cAMP regulated phosphoprotein 19 |
| | member A | |
| FUS56 | zinc finger protein 772 | vomeronasal 1 receptor 1 |
| FUS57 | mediator complex subunit 12 | neuroligin 3 |

TABLE 7

| Neoantigen | | SEQ ID |
|---|---|---|
| ID | Polynucleotide sequence | NO: |
| FUS1 | ATATCTAATAAGATAGCCTTACTGCAAACCTTT | 2 |
| FUS2 | TGGCTAGTTAAACAAAGAGGCTGGGCAAACCTTTTAGAGAA | 4 |
| | GTCAGCTGAGCAAATATGTACAGGTGAATTCAAAGCAAAAG | |
| | CCTCACAAAGT | |
| FUS3 | AACCACGAGACGGCCTTCCAAGGCAAACCTTTTAGAGAAGT | 6 |
| | CAGC | |
| FUS4 | GATGTGGCTGTCATCTGCTCAGGAAGACGTTCAGCCAGGGA | 8 |
| | TCCA | |
| FUS5 | GATGTGGCTGTCATCTGCTCAGGGGTCCCTGTCGCTGATGTG | 10 |
| | AGC | |
| FUS6 | GAAGATGTGGCTGTCATCTGCTCAGAGCTGTTTTTGATAGCC | 12 |
| | AGCCCC | |
| FUS7 | GGTCGTGCAGGCCAGGGAGGAGGCATGATTTCTGTGAGTCA | 14 |
| | GGAATTCATTAAG | |
| FUS8 | GCAGTGCTCAAACTCACCGACCAGCATAATGCAGTGAAGAT | 16 |
| | GGGTGAAGAGAGATGTGTTTCATCTGAAATT | |
| FUS9 | AGTGGCCTTCTGATGAATACTTTAACTGTGCTGATCCTGTAT | 18 |
| | TTCCTGTAT | |
| FUS10 | GCTCCTAAGCCCAAGAAGATGAAGAACCATTTGCTTTTCTGG | 20 |
| FUS11 | GGGAAATCCACACCACCACGTAAGGAAATATTTGATGATGC | 22 |
| | GTCACCT | |
| FUS12 | GCCCGCTGGTTCACTAGAGAACAGGGCAGTGAAAAC | 24 |
| FUS13 | GCGTTCATCTACCACGCTTTCGTTGGGTCAACCAAAACCGCA | 26 |
| | TGGCGTACTACTCTC | |
| FUS14 | CTGGCTAGGCAAATAAGCTTTAAGTCACTTATA | 28 |
| FUS15 | CAGCCAAGAGACAACCACACTGATCAAGTCACTTATATAAG | 30 |
| | AAGAACCTTGATG | |
| FUS16 | CCAAGAGACAACCACACTGATCATGAAAACCCAAGAGGAA | 32 |
| | GCGATGGACAGGGCAGGTGGAAATGCCCAAGCCAAGTCACT | |
| | TATATAAGAAGAACCTTGATG | |
| FUS17 | GCTGCTGGTGTTGTCACTGAGATCGAAGCCGCGGTCAAGGC | 34 |
| | C | |
| FUS18 | AACGCAGTTCGGAGTTATTATGAGGGGACGGAAAATATAGT | 36 |
| | CGCGGTG | |
| FUS19 | AACGCAGTTCGGAGTTATTATGAGACAAATGGAGGCATGAG | 38 |
| | CTTCCTTAGAATTACCCCT | |
| FUS20 | ATCAGCAAGCGACTCAAGAGCAGAAGGAGATCCGGTTGGCA | 40 |
| | GCTAAACCGCGCTGGGAACAGGGGCCTGAGTCCTGGACTAG | |
| | GGCTCTTTCCCCGGGGCTGCTGCAGATGGGGAGGAGCCTAC | |
| | ACCCGCCTCCCGAGTGCTAATCAGACC | |
| FUS21 | CCACCCACCGCCGCCGCCTCCCAGACCTGCCTAGAGCTGGA | 42 |
| | ACGT | |
| FUS22 | GCCTGCGGAGCCTGTGGGGCAGGATTTTTTATCAAGCAGAA | 44 |
| | ATGCATCGAACAACGAGAATCAAGATCACTGAGC | |
| FUS23 | GTGTACCCTGTCTACCAGCCTGTGGTTTCACAAGCTGGTCTG | 46 |
| | GGGGAGCTTTGGCAGTGGGCTTCTGGAAAGCTCCGGGGTTA | |
| | TTGTTGGCGGCGGGGACAATGGCATGCTTATTCTATACAATG | |
| | TGACCCACATCCTGTCTTCGGGGAAGGAGCCTGTGATTGCTC | |
| | AGAAACAGAAGCACACGGGGGCTGTCAGAGCCCTCGACTTG | |
| | AATCCTTTCCAGGGCAACCTCCTGGCTTCAGGGGCCAGCGAT | |
| | TC | |
| FUS24 | TCTGGCCTGGAGGCTATCCAGCGGGGATATGAGACTTTCTAC | 48 |
| | TCGGAA | |
| FUS25 | TCCCCAGAGGACTACACGCTCAAGGCCCAGGGCCCCGGCCA | 50 |
| | GTGCCCA | |
| FUS26 | GCGAGGCACTTGCTAACTCTTGGGACATGCGGAAGCACGTG | 52 |
| | GCCA | |
| FUS27 | CCACAGGAAGAAGTGCCATTCAGGTCAATAACAACGGAGTT | 54 |
| | GTTTCCTTCAATGTGC | |
| FUS28 | CAGATCATCTACGACGAGAACCGGACAATGCAGATCTTCGT | 56 |
| | GAAGACT | |
| FUS29 | GGCGAGCGCGAGCGGCGCGAGAAAACAATGCAGATCTTCGT | 58 |
| | GAAGACT | |
| FUS30 | ATGGATCTGACTGCCATCTACGAGACAATGCAGATCTTCGTG | 60 |
| | AAGACT | |
| FUS31 | CCACCCACCGCCGCCGCCTCCCAGACAATGCAGATCTTCGTG | 62 |
| | AAGACT | |
| FUS32 | TCTTCTGTGACTACCCACTCATCTGGAAATTTGCGAGAATCC | 64 |
| | CCGATTTATCAT | |
| FUS33 | TCTGGCCTGGAGGCTATCCAGCGTGAGAAGCCCTACCACTG | 66 |
| | CAACTGG | |
| FUS34 | GTGGCACCCAGTGGCCTGTACTACTGTGGA | 68 |
| FUS35 | AACAGGGTCACAGCTCTCACCCAGAAC | 70 |
| FUS36 | ATAGAGAAATCAATTTCTGCTAGGATCACAGAACAA | 72 |
| FUS37 | CCAAGTGACTGCTCCATTCGGAAGACTTTGTTGTCGCTGCCC | 74 |
| | GGAGGAGTCGAGACTGGTACCCGGAGGAGCTGTCTCACCAG | |
| | GAGACCACGTCCTGGAAGTGTCCGGGACTCGCGGGACCTGT | |
| | GGCTGCAGACCCCGCCGGCACGCAGGCCCAGAGCTGGCGCA | |
| | CTCCTGAGGATGAGACTCTGGGGGCCC | |
| FUS38 | GGGGAAGTGAAGTGGTCTGTAGGCTCCCGGACGTCCCTGCT | 76 |
| | CCTG | |
| FUS39 | CTCACGGAGAAGAAGGCCTCCGACGTGAGAAGCCCTACCAC | 78 |
| | TGCAACTGGGACGGCTGCGGCTGGAAGTTTGCGCGCTCAGA | |
| | CGAGCTCACGCGCCACTACCGAAAGCACACGGGCCACCGGC | |
| | CATTCCAGTGCCATCTGTGCGATCGTGCCTTCTCGCGCTCCG | |
| | ATCACCTGGCGCTGCACA | |
| FUS40 | TGCCGCGAGCGCGGCCTGCAGGAGGCTGAAGGTGATGTGGC | 80 |
| | CGCCCTC | |
| FUS41 | CCACCCACCGCCGCCGCCTCCCAGGTGAGAAGCCCTACCAC | 82 |
| | TGCAACTGGGACGGCTGCGGCTGGAAGTTTGCGCGCTCAGA | |
| | CGAGCTCACGCGCCACTACCGAAAGCACACGGGCCACCGGC | |
| | CATTCCAGTGCCATCTGTGCGATCGTGCCTTCTCGCGCTCCG | |
| | ATCACCTGGCGCTGCACA | |
| FUS42 | GGTGGCCCTCCTTTTGTAGGCCCGCTTCGGGGGCCGCGTGCC | 84 |
| | CAGCCTAGCCTATCCGGACTGTGCGGGAGGCGGACGGGGAG | |
| | CAGAGACACAATAAAAATTCCCGGTCAAACC | |
| FUS43 | GGTTTGAAACAGCTTCACAAGGCAGGTTATGAAGAAGAAAC | 86 |
| | TCAAAATAACAGGAGTGGCTTATGGAACTACATGGAGCTCT | |
| | GGCAGGCTGCCCTG | |
| FUS44 | CTGGACCAGTGTGTGGAGACCCTCTGCTCCCTCAGCAAGTCG | 88 |
| | GAC | |
| FUS45 | CTCCTTCCACCTGGCCAGGAATCAGCCATTATTGGACCCCCT | 90 |
| | GGAATGCAA | |
| FUS46 | TATGAATGTACTGGATGCAAAACGAGCTTCCCACACAGAAG | 92 |
| | CCAGAGC | |
| FUS47 | CAACATCCTGCTAGACTTGTTAAGTGTGGAACACAA | 94 |
| FUS48 | GGCCTGACTCTGGGAGCACGCACTACTCTGACCCATGGATC | 96 |
| | CCCTGGGCCCAGCCAAGCCACAGTGGCTGTGGCGCCGCTGT | |
| | CTGGCCGGGCTGCTGTTTCAGCTGCTGGTGGCTGTGTGTTTC | |
| | TTCTCCTACCTGCGTGTGTCCCGAGACGATGCCACTGGATCC | |
| | CC | |
| FUS49 | ATGGATCTGACTGCCATCTACGAGTGCTGCAACCTGAGCGT | 98 |
| | GAGCGAG | |
| FUS50 | GTAATTGTGGACCCTATACAGGGATACCTCAGACAATGGCA | 100 |
| | GCTTTTGGAT | |
| FUS51 | CCGCCACTCATCACCAATGTCACGGAAAAGCCGTGGAAACA | 102 |
| | CACCCGGATTGAAATGCCCCTGGCCCGCCTGACCCGACCC | |
| FUS52 | ATGGAACCTACTACCAAAAGAGGTCCTGGGGGCACCATGGT | 104 |
| | TTCGGCGGCAGCCCCCAGC | |
| FUS53 | AGGCCTGGAGTATTCACAGACATTCTT | 106 |
| FUS54 | ATGGATCTGACTGCCATCTACGAGGACTCTGCTGGGCCGCTC | 108 |
| | AGCCATGCC | |
| FUS55 | TTACGCTACCTCATACATCTGAGGAAATGGAAGATAAAG | 110 |
| FUS56 | TTTGCACTTATGGCCTCTCTGGGGATACCTCAGACAATGGCA | 112 |
| | GCT | |
| FUS57 | CGGCAACTTCAACAACAGCTCTCTAGCCTGTCTGGCCCTGAG | 114 |
| | GGAGTCCCCTTTCTGAAGCTGTGGTGCTTGGACGACCTGCTC | |
| | TCTACATTGCTGGGCACCTGTAGGTGTCCCTCGAGAGCTCAG | |
| | TTT | |

Table 8 shows gene origin and amino acid sequences of identified neoantigens that arose from alternative splicing (AS) events. Bolded letters represent sequences from the wild-type protein while regular letters represent mutant sequence resulted from alternative splicing events. Table 9 shows the genomic coordinates of the alternative splicing events and the full gene names. Table 10 shows their corresponding polynucleotide sequences.

TABLE 8

| Neoepitope | | | SEQ ID |
|---|---|---|---|
| ID | Gene | Amino acid sequence | NO: |
| AS1 | ASPM | LKTTNRASVSISQLPFFKIIFLWS | 115 |
| AS2 | ATP8B2 | RLASIYEELLGATAIE | 117 |
| AS3 | BBS9 | SLLSLFPGKTVEITCLQHQKC | 119 |
| AS4 | BTBD3 | TQRLPGHKVSNSCMTGLVLTFTKRDPFHKPVT | 121 |
| | | WCGQLADVRQCMFHSIRERAHPLQRALATLNF | |
| | | FFVSFYTALYLTHPLLTLQTWEVVVTVCFSCW | |
| | | ELCVPCDVLRRTCRGQR | |
| AS5 | C3orf79 | LQAILQPGNFILVNEI | 123 |
| AS6 | CTHRC1 | RQREVVDLVSPREPSRDRRAGGGDLAARPTG | 125 |
| | | RASVWLLGVSVCTAVCRVSCCAGVSCFLFTGE | |
| | | EGPGYACSESWNSEGRPKWEAIVERTCDMGPY | |
| | | NGMCLQG | |
| AS7 | ERC2 | LVHQLKQQVGPPARQTQNRMKLM | 127 |
| AS8 | FRA10AC1 | FHLIAMDAVSFFYFQIFITSRIF | 129 |
| AS9 | GAS2 | GEKILFIRVKFYFSLILFLWEDSEFELGEI | 131 |
| AS10 | PDK1 | MKQFLDFGECGPGPWAFLRGPGRGAAAAAPG | 133 |
| | | RVGAGQLSPEAHPSSSAFPPPAP | |
| AS11 | TSG101 | QLKKMVSKVRLRRARLPGRAHRSLPRPVESVP | 135 |
| | | AQPSKLPRRAGSPGAVLSDLLRTPPRGACRLVR | |
| | | ESPYGSWEGGGRRQSTKAWSGGYLTPAAHPPS | |
| | | SLPLSLELSVGQCSFSFVFINTNKGSYLVVDLRQ | |
| | | SSTPVPI | |
| AS12 | XRN1 | DALIQNQHVSTVVCIVSFRIDKLLKY | 137 |
| AS13 | ANKRD36B | GGNNSNQQLDDARNKA | 139 |
| AS14 | ARMC2 | NSGHLLVQRWSLALSPRPECSGMISAHCNLHL | 141 |
| | | PGSSSSPASASQVDGITGTCRHFFNDVFVNGKE | |
| | | TAIFYLWGFLPIKELPAA | |
| AS15 | B4GALT2 | YGVYVINQKGHSGCIWSMDGEGRLETAWPV | 143 |
| | | WKRGRCPGKRR | |
| AS16 | CAMSAP1 | SRAPIKMSVLGQGHSRCLLHCLCLVAPMAFPV | 145 |
| | | RLRVPCGG | |
| AS17 | DCC | SATTRSITGQDILSLEKERR | 147 |
| AS18 | FCRLB | LTALLLLASTPTP | 149 |
| AS19 | GABRR2 | FSMRPAFGETALGCKKW | 151 |
| AS20 | IGLL5 | SSLRSLWGSRLLLQPSP | 153 |
| AS21 | KLHL14 | QLPPMQERATISLQGTEYPKY | 155 |
| AS22 | KMT2C | TFTHLKQQLSLLPLMEPIIGVNFAHFLPYGSGQ | 157 |
| | | FNSGNRLLGTFGSATLEGVSDYYSQLIYKVCFS | |
| | | AKVLSCFIFG | |
| AS23 | KMT2C | CKVCQNCKNPKMARAS | 159 |
| AS24 | ST3GAL6 | DLFDEFDNSRLVSRSWFFFYLRNKWNLALGF | 161 |
| AS25 | TBCEL | RSISLHKSDPSLLSPN | 163 |
| AS26 | AGFG1 | FLQKHGNENTVLMLETLSEIRYGYVSCVCCPL | 165 |
| | | LSPYGRCLN | |
| AS27 | AKAP9 | PPEILSNESLHGGIWPLRFWLPHWIISMERSQY | 167 |
| | | GTQTRKTVYEN | |
| AS28 | ARHGAP26 | EAMDGREPHLLDKNDDRSISW | 169 |
| AS29 | ARHGAP42 | SLLIGALRITWKREAGRKSFFKCGLRVHMWK | 171 |
| | | GLQFIEITRRNWMLIFQSCSLFGTSQYRSLTKKC | |
| | | QLCFRFKRVSSKGSDITDKREVTH | |
| AS30 | BLNK | TVPASQKLSCSLDPKHLRQFHL | 173 |
| AS31 | BMP6 | MVMSFVNLALARLTQLQQESPARVWPSWEEG | 175 |
| | | SSVRFSPLNCRLLPPNLECSFPTETFRESDSGWE | |
| | | L | |
| AS32 | CARMIL1 | LEDLDTCMKPHAFQHAMMTFASTTFRLCQKS | 177 |
| | | SKLSLLSS | |
| AS33 | CFAP54 | FGTSHMMVRHF | 179 |
| AS34 | CNTN5 | GHFESIRAVLFHWIPVTTLLVSQVIYK | 181 |
| AS35 | DYRK1A | MHTGTISQHCQLLDCSLGAILHIGYTK | 183 |
| AS36 | EPHA6 | EIEGSCHGGMITMDIKMGSYSCDYLKHSETLS | 185 |
| | | AMVVSCGISFLFLFPRKKIAKLIFKEQVP | |
| AS37 | FAM13A | CEIMPLQSYILL | 187 |
| AS38 | FEM1C | VNRKSVKGFHVAHLPSA | 189 |
| AS39 | GOSR2 | FQDKYFMIATN | 191 |
| AS40 | ITGA8 | VAQVEIRGLEPPVHDQ | 193 |
| AS41 | KMT2A | GGGGGSGELTTQIPCSWRTKGHIHDKKTEPFR | 195 |
| | | LLAWSWCLNVS | |
| AS42 | LARP1B | VAPSQSRQEECWRHKMSW | 197 |
| AS43 | LYSMD3 | IALQYCCTCSISQ | 199 |
| AS44 | NBEA | SAVDDMIAAFPFKRA | 201 |
| AS45 | NEDD9 | MWTRKARGTTICDRRRQICRWRE | 203 |
| AS46 | NLGN4X | INYRLGILDNVS | 205 |
| AS47 | NRG3 | TSTSPKFPVFIFRLVCWVRTELVLR | 207 |
| AS48 | NSRP1 | MAIPGRQNIVLRSHRKRVGI | 209 |
| AS49 | OXR1 | EAEFDKTTRTFLQIFNRKRGKIQ | 211 |
| AS50 | PDZRN4 | KLLYEVSQKMVL | 213 |
| AS51 | PHC3 | WAFIHSLPALLGIFSYPPSR | 215 |
| AS52 | PRDM5 | SSLQEHRKLLS | 217 |
| AS53 | RUFY4 | RFSRRYPCSARTSLGREESEPPGEAAMWRWRL | 219 |
| | | WRGRASGHPSGEGRPPETNQGHSTGR | |
| AS54 | SETBP1 | ERSHSKKKDGLVRNDGPGFPPMFSTSENTNLD | 221 |
| AS55 | TYW5 | AGDVLFIPGISLKVHKVQHSKVINICN | 223 |
| AS56 | ADGRE5 | IENEGGQVRSRPAPS | 225 |
| AS57 | ALOX5AP | LISVVQNGKESPSLREEQKGRFSLMVVWKSGL | 227 |
| | | NNCVCVCACTNTFTLSLFSSFYLNV | |
| AS58 | ATP11B | EHQRNSSKVFYGVIDCVIKETITWHD | 229 |
| AS59 | CASP10 | DSLPKTEMVSGSYRMGLCEHCLNQ | 231 |
| AS60 | DDX3Y | RPRKDTWKVRDPALRYTPLLIQSLDSRLAESC | 233 |
| | | AVFIWYCIFTLGDTCRSRPVEN | |
| AS61 | DOCK8 | PVGSVSFQVGCVQLFP | 235 |
| AS62 | FCAR | QRIQAQEGKCPVNLSQPL | 237 |
| AS63 | GAB1 | NLKPDRKGKESMAK | 239 |
| AS64 | ITGA8 | DLNQDGYNGNLNQSVEVIYVSSPLHKGDHAC | 241 |
| | | FKVFSMVCVGAVIITDF | |
| AS65 | MORF4L1 | APHLLRLFGNMSCRK | 243 |
| AS66 | MTCH1 | SLKKVVKEVGVESWRGRGAWRGA | 245 |
| AS67 | MTMR14 | PVILFKGKVRPIP | 247 |
| AS68 | NFKB1 | QILEQPKQVRLKGWDFKC | 249 |
| AS69 | PARP8 | IATGAQVVTQDTNYFLFCFEKFVPVH | 251 |
| AS70 | PDZRN4 | LLYEVSQVSAGSDTSN | 253 |
| AS71 | PPFIBP2 | WKLKATKVNGTPDAYGESMRALWGGSAFQL | 255 |
| | | GGFQ | |
| AS72 | RGPD2 | SPAIYKLKVNIQNIKGENLRHNHF | 257 |
| AS73 | SMC5 | DMEVFLKEASTNQHNTLIHLTLTFST | 259 |
| AS74 | TBC1D23 | LQANQLQGVSKGNPVC | 261 |
| AS75 | TFG | LTDDQVSGKLVSNSFTPFVSFIFPFFPFLLSLIEE | 263 |
| AS76 | TLE4 | QRDRDSIKVGLKIYRLRNGLKGL | 265 |
| AS77 | TRAF3 | KRYGCVFQVSIRHLSFPVTDILP | 267 |
| AS78 | UBE2B | EGTPFEDGKSYSLCFL | 269 |
| AS79 | USP38 | SMTQALCRYFFMTLLMVIVDLGKRKYFVFCE | 271 |
| AS80 | WASHC4 | LLDGMIFQVSRSVSDCNSGAHIRRQSSEL | 273 |
| AS81 | ZNF215 | EIPRKTIFGKNQVDMRP | 275 |
| AS82 | ANKRD36 | SQKQPAEKCLLRNNQLRRLQVTRKILFQI | 277 |
| AS83 | ANKRD36 | SRKKPALKCLLRNNQH | 279 |
| AS84 | ANKRD36 | SQKQPALKCLLRNNRP | 281 |
| AS85 | ANKRD36C | SEQPPGLKCLLGKKQP | 283 |
| AS86 | ATP10B | VYYLYKNVAYKGSDID | 285 |
| AS87 | BMP6 | QLSVVTRDDYNSSELK | 287 |
| AS88 | CADPS2 | LEDKELGRISVCPWTEGLEKMEKTLLCSSSG | 289 |
| AS89 | CCDC88A | ENQRLSKKDLVSEKLK | 291 |
| AS90 | CSAG1 | MSATTGSQDNPEGKRDPSRKFQEQKALPKRPP | 293 |
| | | LQKNLRNGVGQHYPATLTSRTRNSIKMRHSRT | |
| | | TRARRPPPSPVLPWAASNSQARPETSSLSEKSPE | |
| | | V | |
| AS91 | KMT2B | QDLATEDTGSVTK | 295 |
| AS92 | MSI2 | DPTTKRSRLTPKLHFLVERNPRWSQEQRKYL | 297 |
| AS93 | TJP1 | ILAQKKKDELSS | 299 |
| AS94 | TTLL7 | AKFWSDISINRAPSFG | 301 |
| AS95 | WDR64 | LDPPHDEKALACPQWTLLWIFWTAKAL | 303 |
| AS96 | ANKRD28 | KKEDVNFQVKQLIH | 305 |
| AS97 | ANKRD28 | GNVLVRYVSLYVYYILLN | 307 |
| AS98 | ARHGAP26 | QRSFIKAASKSFLSQF | 309 |
| AS99 | DST | FSGRSRSRGKTRRDGFLRATRNN | 311 |
| AS100 | HDAC9 | RESKRHQGKRWTLFPHR | 313 |
| AS101 | IZUMO4 | HLAIPAKISECRSPAQSRLPRQRDPGAGRSPGF | 315 |
| | | SSCPQPGRSWTKWRQQCTR | |
| AS102 | MAGEC3 | LDLANPQGKGPKGELRDFAPRTEEAPVCPALP | 317 |
| AS103 | PLEKHG2 | RDLRSIVEVRRADTRGQWVPRPAPWPP | 319 |
| AS104 | QRICH2 | LLTEMDNKVRQGHGGALHPTALPPGSGQHHL | 321 |
| | | LLGQPPTCFLRGPLAPLSPPQLDRLELDP | |
| AS105 | ST3GAL6 | MLRRCPCVTNVVLAL | 323 |
| AS106 | TEX14 | AGKERSTQVRVGPTL | 325 |
| AS107 | TEX9 | AGRSLCLTVSSTPGSWGASGFRRLGRLTSFNF | 327 |
| | | GSESLWLFYREAAFQGLRSRHPFSNPLHLDPTS | |
| | | SPWRKNISV | |
| AS108 | ACIN1 | VEDEEKKEAGTHFIHLTGTTVSAGVPEEMPAT | 329 |
| | | TLRREVF | |
| AS109 | ADAMTS15 | TAILFTRQQSKALPSLGITNRHGFWAGQGNTV | 331 |
| | | MHGRDSDGKQVKGTVVILAQGAFHQ | |
| AS110 | CCDC88A | NASLHEVKDFFVPFPLPLLHLTSLKRNKLFN | 333 |
| AS111 | CD55 | GTTRLLSGEDFS | 335 |
| AS112 | CFAP54 | SEMVAHERYVFEATGNDSFRTLCLLMK | 337 |
| AS113 | CLCC1 | IIMALAILDLFAVLKIEAHKQLLLKSFCYGAG | 339 |
| | | K | |
| AS114 | CNTN5 | QIRVPSYSDEG | 341 |
| AS115 | CTHRC1 | RQREVVDLVKKDLVMRAVSLGTQRGDQNGR | 343 |
| | | PSWSGHAIWARIMECAYKGQQECLVETGALGP | |
| | | MAFRVHLGSQVGMDSKEKRGNV | |
| AS116 | FCRL5 | VHLDFSSAEMGFPHAAQANVELLGSSDLLT | 345 |
| AS117 | GAS2 | GEKILFIRGLHGCDYISTAKAKQTFKMLHNK | 347 |
| | | HVM | |
| AS118 | IARS | GFFETEMAVPFFSRKIRKVRRAIMKWTFCCYF | 349 |
| | | Y | |
| AS119 | NBEA | DSQVETTVWGVGNRQEWREILYGSTQNFASL | 351 |
| | | L | |
| AS120 | NCAM1 | WTRPEKQELNSSCCSLIL | 353 |
| AS121 | OGT | LGRLEEAKSFETEEN | 355 |
| AS122 | OGT | LGRLEEAKVPLKMLLF | 357 |
| AS123 | PCNX3 | WLLRTWERADSGL | 359 |
| AS124 | PDZRN4 | LVARPEIQIFMEEDVMKVE | 361 |
| AS125 | PRDM5 | RHQENVHTVLVHGKVKGLFYI | 363 |
| AS126 | RAB2B | IMLIGNKSNCQKQLLRDMKILNEFYHGLILTLQ | 365 |
| | | LS | |
| AS127 | ST3GAL6 | PKVFPKNQVISLIPKAQEDPEHT | 367 |
| AS128 | STARD5 | GWKICREGIGPVMLTMTSGEWSFSFLEAICGV | 369 |
| | | SREPVPRRRHCIWDTRGGVGLCEASCWRPTSE | |
| | | VG | |
| AS129 | TET1 | IDPSSPLHTYYERITKGRNPERRYMKPERISPG | 371 |
| | | HEAMEKNLEDNL | |
| AS130 | TMTC2 | NMADMLYNFLSQTSAAAKLNLQFEMITAVFR | 373 |
| | | LNRCAECLGLLLQENS | |
| AS131 | USP25 | QILQQALKKLPFSLCITECETIAYHLARDSNGN | 375 |
| | | LEL | |
| AS132 | USP25 | QILQQALKIAFLKVVVPLKLHPYSFCH | 377 |
| AS133 | WNT10A | YESPIFSRDRVNPGALSGSLGQARAACWRTLG | 379 |
| | | SLKPGGEFRAAEQRRAGWVPGAPGFTCSLISSR | |
| | | FPRERFCLRHRSSWRGARRVQCVCPGQTEGLW | |
| | | L | |
| AS134 | XPO1 | FSQNMNTKMIRGTERKQQYYGLQILE | 381 |
| AS135 | ZBP1 | GEPGEDAGIGRPEEREREEEMADQ | 383 |
| AS136 | ZNF592 | PPGGHSPQDLSIREIFCLGHKEVC | 385 |
| AS137 | ZNF680 | NYRNLVFLDPCFQENL | 387 |
| AS138 | BTBD3 | TQRLPGHKVSNSCMTGLVLTFTKRDPFHKPVT | 421 |
| | | WCGQLADVRQCMFHSIRERAHPLQRALATLNF | |
| | | FFVSFYTALYLTHPLLTLQTWEVVVTGQESRM | |
| | | FTLLLEISYVSYYCMYLVMRGHVH | |

TABLE 9

| Neo- | | |
|---|---|---|
| epitope | AS genomic | |
| ID | coordinate | Full gene name |
| AS1 | ASPM.197087073 | abnormal spindle microtubule assembly |
| AS2 | ATP8B2.154344039 | ATPase phospholipid transporting 8B2 |
| AS3 | BBS9.33358079 | Bardet-Biedl syndrome 9 |
| AS4 | BTBD3.11919423 | BTB domain containing 3 |
| ASS | C3orf79.153486182 | chromosome 3 open reading frame 79 |
| AS6 | CTHRC1.103372064 | collagen triple helix repeat containing 1 |
| AS7 | ERC2.55734750 | ELKS/RAB6-interacting/CAST family member 2 |
| AS8 | FRA10AC1.93695770 | FRA10A associated CGG repeat 1 |
| AS9 | GAS2.22756255 | growth arrest specific 2 |
| AS10 | PDK1.172556874 | pyruvate dehydrogenase kinase 1 |
| AS11 | TSG101.18525661 | tumor susceptibility 101 |
| AS12 | XRN1.142376697 | 5′-3′ exoribonuclease 1 |
| AS13 | ANKRD36B.97511347 | ankyrin repeat domain 36B |
| AS14 | ARMC2.108920209 | armadillo repeat containing 2 |
| AS15 | B4GALT2.43982817 | beta-1,4-galactosyltransferase 2 |
| AS16 | CAMSAP1.135882376 | calmodulin regulated spectrin associated |
| | | protein 1 |
| AS17 | DCC.53388643 | DCC netrin 1 receptor |
| AS18 | FCRLB.161723047 | Fc receptor like B |
| AS19 | GABRR2.89272016 | gamma-aminobutyric acid type A receptor |
| | | rho2 subunit |
| AS20 | IGLL5.22893697 | immunoglobulin lambda like polypeptide 5 |
| AS21 | KLHL14.32680935 | kelch like family member 14 |
| AS22 | KMT2C.152157925 | lysine methyltransferase 2C |
| AS23 | KMT2C.152263362 | lysine methyltransferase 2C |
| AS24 | ST3GAL6.98784799 | ST3 beta-galactoside alpha-2,3- |
| | | sialyltransferase 6 |
| AS25 | TBCEL.121057508 | tubulin folding cofactor E like |
| AS26 | AGFG1.227510727 | ArfGAP with FG repeats 1 |
| AS27 | AKAP9.92043282 | A-kinase anchoring protein 9 |
| AS28 | ARHGAP26.142961909 | Rho GTPase activating protein 26 |
| AS29 | ARHGAP42.100738266 | Rho GTPase activating protein 42 |
| AS30 | BLNK.96257140 | B-cell linker |
| AS31 | BMP6.7727950 | bone morphogenetic protein 6 |
| AS32 | CARMIL1.25563202 | capping protein regulator and myosin 1 |
| | | linker 1 |
| AS33 | CFAP54.96645158 | cilia and flagella associated protein 54 |
| AS34 | CNTN5.100202717 | contactin 5 |
| AS35 | DYRK1A.37421774 | dual specificity tyrosine phosphorylation |
| | | regulated kinase 1A |
| AS36 | EPHA6.97028432 | EPH receptor A6 |
| AS37 | FAM13A.88822753 | family with sequence similarity 13 member A |
| AS38 | FEM1C.115529578 | fem-1 homolog C |
| AS39 | GOSR2.46948943 | golgi SNAP receptor complex member 2 |
| AS40 | ITGA8.15580920 | integrin subunit alpha 8 |
| AS41 | KMT2A.118439024 | lysine methyltransferase 2A |
| AS42 | LARP1B.128168818 | La ribonucleoprotein domain family member |
| | | 1B |
| AS43 | LYSMD3.90520461 | LysM domain containing 3 |
| AS44 | NBEA.35042719 | Neurobeachin |
| AS45 | NEDD9.11272293 | neural precursor cell expressed, |
| | | developmentally down-regulated 9 |
| AS46 | NLGN4X.5936111 | neuroligin 4, X-linked |
| AS47 | NRG3.81880826 | neuregulin 3 |
| AS48 | NSRP1.30117302 | nuclear speckle splicing regulatory |
| | | protein 1 |
| AS49 | OXR1.106690929 | oxidation resistance 1 |
| AS50 | PDZRN4.41462796 | PDZ domain containing ring finger 4 |
| AS51 | PHC3.170102305 | polyhomeotic homolog 3 |
| AS52 | PRDM5.120808021 | PR/SET domain 5 |
| AS53 | RUFY4.218089687 | RUN and FYVE domain containing 4 |
| AS54 | SETBP1.44758802 | SET binding protein 1 |
| AS55 | TYW5.199934863 | tRNA-yW synthesizing protein 5 |
| AS56 | ADGRE5.14405748- | adhesion G protein-coupled receptor E5 |
| | QVRSRPAPS* | |
| AS57 | ALOX5AP.30735508- | arachidonate 5-lipoxygenase activating |
| | LFSSFYLNV* | protein |
| AS58 | ATP11B.182857878- | ATPase phospholipid transporting 11B |
| | IKETITWHD* | |
| AS59 | CASP10.201187706- | caspase 10 |
| | GLCEHCLNQ* | |
| AS60 | DDX3Y.12905705- | DEAD-box helicase 3, Y-linked |
| | TCRSRPVEN* | |
| AS61 | DOCK8.434783- | dedicator of cytokinesis 8 |
| | QVGCVQLFP* | |
| AS62 | FCAR.54875330- | Fc fragment of IgA receptor |
| | CPVNLSQPL* | |
| AS63 | GAB1.143440079- | GRB2 associated binding protein 1 |
| | RKGKESMAK* | |
| AS64 | ITGA8.15647051- | integrin subunit alpha 8 |
| | VGAVIITDF* | |
| AS65 | MORF4L1.78894058- | mortality factor 4 like 1 |
| | LFGNMSCRK* | |
| AS66 | MTCH1.36978155- | mitochondrial carrier 1 |
| | STSCKWMVR* | |
| AS67 | MTMR14.9662267- | myotubularin related protein 14 |
| | FKGKVRPIP* | |
| AS68 | NFKB1.102533845- | nuclear factor kappa B subunit 1 |
| | RLKGWDFKC* | |
| AS69 | PARP8.50815432- | poly(ADP-ribose) polymerase family |
| | RIATGIKTE* | member 8 |
| AS70 | PDZRN4.41437739- | PDZ domain containing ring finger 4 |
| | VSAGSDTSN* | |
| AS71 | PPFIBP2.7620935- | PPFIA binding protein 2 |
| | SAFQLGGFQ* | |
| AS72 | RGPD2.86953498- | RANBP2-like and GRIP domain containing 2 |
| | GENLRHNHF* | |
| AS73 | SMC5.70305247- | structural maintenance of chromosomes 5 |
| | IHLTLTFST* | |
| AS74 | TBC1D23.100297923- | TBC1 domain family member 23 |
| | GVSKGNPVC* | |
| AS75 | TFG.100732508- | TRK-fused gene |
| | PFLLSLIEE* | |
| AS76 | TLE4.79654059- | transducin like enhancer of split 4 |
| | RLRNGLKGL* | |
| AS77 | TRAF3.102889560- | TNF receptor associated factor 3 |
| | SFPVTDILP* | |
| AS78 | UBE2B.134376669- | ubiquitin conjugating enzyme E2 B |
| | GKSYSLCFL* | |
| AS79 | USP38.143187826- | ubiquitin specific peptidase 38 |
| | KRKYFVFCE* | |
| AS80 | WASHC4.105122118- | WASH complex subunit 4 |
| | HIRRQSSEL* | |
| AS81 | ZNF215.6943546- | zinc finger protein 215 |
| | GKNQVDMRP* | |
| AS82 | ANKRD36.97179766- | ankyrin repeat domain 36 |
| | 97181598 | |
| AS83 | ANKRD36.97194754- | ankyrin repeat domain 36 |
| | 97211546 | |
| AS84 | ANKRD36.97142668- | ankyrin repeat domain 36 |
| | 97144518 | |
| AS85 | ANKRD36C.95895590- | ankyrin repeat domain 36C |
| | 95903053 | |
| AS86 | ATP10B.160589696- | ATPase phospholipid transporting 10B |
| | 160602577 | |
| AS87 | BMP6.7861599- | bone morphogenetic protein 6 |
| | 7879074 | |
| AS88 | CADPS2.122513315- | calcium dependent secretion activator 2 |
| | 122581179 | |
| AS89 | CCDC88A.55332693- | coiled-coil domain containing 88A |
| | 55339464 | |
| AS90 | CSAG1.152727863- | chondrosarcoma associated gene 1 |
| | 152732445 | |
| AS91 | KMT2B.35722718- | lysine methyltransferase 2B |
| | 35723447 | |
| AS92 | MSI2.57257547- | musashi RNA binding protein 2 |
| | 57262151 | |
| AS93 | TJP1.29732815- | tight junction protein 1 |
| | 29734274 | |
| AS94 | TTLL7.83929230- | tubulin tyrosine ligase like 7 |
| | 83937852 | |
| AS95 | WDR64.241780062- | WD repeat domain 64 |
| | 241787849 | |
| AS96 | ANKRD28.15781656- | ankyrin repeat domain 28 |
| | 15795222 | |
| AS97 | ANKRD28.15795307- | ankyrin repeat domain 28 |
| | 15796395 | |
| AS98 | ARHGAP26.143056087- | Rho GTPase activating protein 26 |
| | 143057641 | |
| AS99 | DST.56953820- | Dystonin |
| | 56954406 | |
| AS100 | HDAC9.18590502- | histone deacetylase 9 |
| | 18591515 | |
| AS101 | IZUMO4.2097163- | IZUMO family member 4 |
| | 2097251 | |
| AS102 | MAGEC3.141895408- | MAGE family member C3 |
| | 141895484 | |
| AS103 | PLEKHG2.39415261- | pleckstrin homology and RhoGEF domain |
| | 39415338 | containing G2 |
| AS104 | QRICH2.76280155- | glutamine rich 2 |
| | 76280286 | |
| AS105 | ST3GAL6.98782969- | ST3 beta-galactoside alpha-2,3- |
| | 98784944 | sialyltransferase 6 |
| AS106 | TEX14.58621787- | testis expressed 14, intercellular bridge |
| | 58622846 | forming factor |
| AS107 | TEX9.56365478- | testis expressed 9 |
| | 56365578 | |
| AS108 | ACIN1.23067314- | apoptotic chromatin condensation inducer 1 |
| | 23068008 | |
| AS109 | ADAMTS15.130456502- | ADAM metallopeptidase with |
| | 130458465 | thrombospondin type 1 motif 15 |
| AS110 | CCDC88A.55297925- | coiled-coil domain containing 88A |
| | 55298414 | |
| AS111 | CD55.207345733- | Complement decay-accelerating factor |
| | 207347256 | |
| AS112 | CFAP54.96638664- | cilia and flagella associated protein 54 |
| | 96638718 | |
| AS113 | CLCC1.108938166- | chloride channel CLIC like 1 |
| | 108938216 | |
| AS114 | CNTN5.100352805- | contactin 5 |
| | 100352912 | |
| AS115 | CTHRC1.103371967- | collagen triple helix repeat containing 1 |
| | 103372064 | |
| AS116 | FCRL5.157546188- | Fc receptor like 5 |
| | 157546283 | |
| AS117 | GAS2.22770929- | growth arrest specific 2 |
| | 22770982 | |
| AS118 | IARS.92255535- | isoleucyl-tRNA synthetase |
| | 92255620 | |
| AS119 | NBEA.35095996- | Neurobeachin |
| | 35096101 | |
| AS120 | NCAM1.113221738- | neural cell adhesion molecule 1 |
| | 113224044 | |
| AS121 | OGT.71544734- | O-linked N-acetylglucosamine (GlcNAc) |
| | 71545037 | transferase |
| AS122 | OGT.71544997- | O-linked N-acetylglucosamine (GlcNAc) |
| | 71545037 | transferase |
| AS123 | PCNX3.65635969- | Pecanex-like protein 3 |
| | 65636031 | |
| AS124 | PDZRN4.41564311- | PDZ domain containing ring finger 4 |
| | 41564348 | |
| AS125 | PRDM5.120812551- | PR/SET domain 5 |
| | 120812814 | |
| AS126 | RAB2B.21466652- | RAB2B, member RAS oncogene family |
| | 21466752 | |
| AS127 | ST3GAL6.98789807- | ST3 beta-galactoside alpha-2,3- |
| | 98791015 | sialyltransferase 6 |
| AS128 | STARD5.81323802- | StAR related lipid transfer domain |
| | 81323835 | containing 5 |
| AS129 | TET1.68673401- | tet methylcytosine dioxygenase 1 |
| | 68673487 | |
| AS130 | TMTC2.82964071- | transmembrane and tetratricopeptide |
| | 82964160 | repeat containing 2 |
| AS131 | USP25.15764238- | ubiquitin specific peptidase 25 |
| | 15764297 | |
| AS132 | USP25.15764805- | ubiquitin specific peptidase 25 |
| | 15764884 | |
| AS133 | WNT10A.218884155- | Wnt family member 10A |
| | 218884323 | |
| AS134 | XPO1.61525492- | exportin 1 |
| | 61525521 | |
| AS135 | ZBP1.57605234- | Z-DNA binding protein 1 |
| | 57605602 | |
| AS136 | ZNF592.84800773- | zinc finger protein 592 |
| | 84801603 | |
| AS137 | ZNF680.64544077- | zinc finger protein 680 |
| | 64544201 | |
| AS138 | BTBD3.11919177- | BTB domain containing 3 |
| | 11919717 | |

TABLE 10

| Neoepitope | | SEQ ID |
|---|---|---|
| ID | Polynucleotide sequence | NO: |
| AS1 | CTGAAGACAACAAATAGAGCCTCTGTAAGTATTAGTCAGTT | 116 |
| | GCCTTTCTTTAAGATTATTTTTCTGTGGTCT | |
| AS2 | AGGCTGGCTAGCATCTATGAGGAGCTGCTGGGTGCAACGG | 118 |
| | CCATTGAG | |
| AS3 | AGTCTTCTTAGTCTCTTCCCAGGTAAGACTGTTGAAATAAC | 120 |
| | ATGCCTGCAGCATCAAAAATGC | |
| AS4 | ACTCAACGGTTGCCAGGACACAAAGTAAGCAACAGCTGCA | 122 |
| | TGACCGGTTTAGTCCTGACGTTTACAAAGAGGGACCCTTTC | |
| | CATAAGCCTGTAACTTGGTGTGGGCAGCTTGCCGATGTCAG | |
| | GCAGTGCATGTTTCACTCGATTAGGGAGAGAGCGCACCCTC | |
| | TCCAGAGGGCTTTGGCCACGCTTAATTTTTTCTTTGTTTCCT | |
| | TCTATACTGCTTTATATCTCACACATCCCCTCTTAACTCTCC | |
| | AGACATGGGAAGTTGTTGTGACAGTATGTTTTAGCTGTTGG | |
| | GAGCTCTGTGTTCCATGCGATGTTTTACGGAGAACTTGCAG | |
| | AGGACAAAGA | |
| AS5 | TTGCAGGCAATTTTGCAACCAGGTAACTTCATTCTGGTCAA | 124 |
| | CGAGATT | |
| AS6 | CGGCAGAGGGAGGTGGTGGACCTGGTGAGTCCGAGGGAGC | 126 |
| | CGAGCCGGGACCGCCGCGCTGGTGGAGGGGACCTGGCCGC | |
| | GCGCCCCACGGGCAGGGCGTCAGTCTGGCTGTTGGGGGTG | |
| | TCTGTCTGTACAGCTGTGTGTCGTGTGTCTTGCTGCGCCGG | |
| | GGTGTCATGCTTTTTATTTACAGGTGAAGAAGGACCTGGTT | |
| | ATGCGTGCAGTGAGTCTTGGAACTCAGAGGGGAGACCAAA | |
| | ATGGGAGGCCATCGTGGAGCGGACATGCGATATGGGCCCG | |
| | TATAATGGAATGTGCTTACAAGGG | |
| AS7 | CTAGTACATCAATTAAAGCAGCAGGTGGGGCCTCCTGCAA | 128 |
| | GACAGACCCAGAACAGAATGAAGTTGATG | |
| AS8 | TTTCATCTCATAGCTATGGATGCTGTATCCTTTTTTTATTTC | 130 |
| | CAGATTTTTATAACTAGTAGAATATTT | |
| AS9 | GGAGAAAAGATCCTCTTCATTAGGGTAAAGTTTTACTTTTC | 132 |
| | ACTTATCTTGTTTTTATGGGAAGATTCAGAATTTGAGTTAG | |
| | GGGAAATA | |
| AS10 | ATGAAGCAGTTCCTGGACTTCGGTGAGTGCGGCCCGGGAC | 134 |
| | CTTGGGCCTTTTTGCGCGGTCCCGGGCGGGGAGCTGCGGCC | |
| | GCTGCCCCAGGCCGGGTCGGCGCCGGCCAGCTCTCGCCTGA | |
| | GGCGCACCCCTCCTCCTCAGCGTTTCCGCCCCCAGCGCCT | |
| AS11 | CAGCTCAAGAAAATGGTGTCCAAGGTGAGGCTGCGACGCG | 136 |
| | CTCGCCTCCCAGGGCGCGCCCACCGCTCCCTTCCGCGCCCT | |
| | GTCGAGTCCGTCCCGGCCCAGCCAAGCAAGCTTCCCAGAC | |
| | GGGCCGGAAGCCCCGGTGCAGTCCTTAGCGACCTCCTCAG | |
| | AACCCCGCCCCGAGGCGCCTGTCGCCTGGTGCGGGAATCCC | |
| | CGTACGGGAGCTGGGAGGGTGGGGGACGGCGACAGTCAAC | |
| | AAAGGCGTGGAGCGGAGGCTACCTGACACCTGCCGCCCAC | |
| | CCGCCCTCCTCTCTTCCACTGAGTTTGGAGCTGTCTGTGGG | |
| | GCAGTGTAGTTTTTCGTTTGTTTTTATAAACACAAACAAGG | |
| | GATCATACTTAGTTGTAGATCTGAGGCAATCCTCTACTCCT | |
| | GTCCCAATC | |
| AS12 | GATGCTTTAATACAGAACCAGCATGTGAGTACAGTAGTTTG | 138 |
| | TATTGTTTCATTTAGAATTGATAAACTTTTAAAATAT | |
| AS13 | GGAGGAAATAATTCAAATCAGCAACTGGATGATGCTCGCA | 140 |
| | ACAAAGCT | |
| AS14 | AATTCGGGCCACTTGCTAGTCCAGAGATGGAGTCTTGCTCT | 142 |
| | GTCGCCCAGGCCAGAGTGCAGTGGCATGATCTCAGCTCACT | |
| | GCAACCTCCACCTCCCGGGTTCAAGCAGTTCTCCTGCCTCA | |
| | GCCTCCCAAGTAGATGGGATTACAGGCACGTGCCGCCACTT | |
| | TTTTAATGATGTATTTGTTAATGGGAAGGAGACAGCTATTT | |
| | TCTACTTATGGGGCTTCCTTCCTATAAAAGAATTGCCAGCA | |
| | GCA | |
| AS15 | TACGGCGTCTATGTCATCAACCAGAAAGGTCACTCTGGTTG | 144 |
| | CATATGGAGCATGGATGGAGAGGGGAGATTAGAGACAGCG | |
| | TGGCCCGTTTGGAAGAGAGGACGATGCCCAGGAAAGAGGC | |
| | GG | |
| AS16 | AGTCGCGCACCCATAAAAATGAGCGTTCTGGGTCAGGGCC | 146 |
| | ATTCTCGGTGCCTCCTGCACTGCCTTTGTTTGGTTGCTCCCA | |
| | TGGCGTTCCCGGTGAGACTGCGGGTGCCGTGCGGTGGT | |
| AS17 | AGTGCCACCACCAGGTCTATAACCGGACAAGATATCTTGA | 148 |
| | GCCTGGAGAAGGAAAGAAGA | |
| AS18 | CTGACAGCCCTTCTGCTCCTGGCTTCCACTCCAACCCCC | 150 |
| AS19 | TTCAGCATGAGACCCGCCTTCGGAGAGACAGCCCTGGGAT | 152 |
| | GTAAAAAGTGG | |
| AS20 | TCCAGCCTGCGGAGCCTGTGGGGCAGCAGGCTCCTGCTCCA | 154 |
| | GCCCAGCCCC | |
| AS21 | CAACTTCCACCCATGCAGGAAAGAGCTACGATATCCTTACA | 156 |
| | AGGTACCGAATATCCCAAATAT | |
| AS22 | ACGTTTACCCACTTGAAACAGCAGCTCTCTCTGCTCCCTCT | 158 |
| | AATGGAACCAATCATTGGAGTGAACTTTGCGCACTTTCTTC | |
| | CTTATGGCAGTGGCCAATTTAATAGTGGGAATCGACTTCTA | |
| | GGAACTTTTGGCAGTGCTACCCTGGAAGGGGTTTCGGACTA | |
| | CTATTCTCAGTTGATCTACAAGGTATGTTTCTCTGCCAAGGT | |
| | GTTGTCTTGCTTCATCTTTGGG | |
| AS23 | TGCAAAGTGTGCCAGAACTGCAAGAATCCTAAAATGGCTA | 160 |
| | GAGCAAGT | |
| AS24 | GATCTCTTTGATGAGTTTGACAACTCTAGACTTGTAAGTCG | 162 |
| | CAGTTGGTTCTTTTTCTACTTGCGCAACAAGTGGAATTTGG | |
| | CACTGGGTTTC | |
| AS25 | CGATCCATCAGCCTCCACAAGTCAGATCCATCTCTCCTGTC | 164 |
| | ACCAAAT | |
| AS26 | TTCTTACAAAAACATGGAAATGAAAACACAGTTCTGATGTT | 166 |
| | AGAGACCTTAAGTGAAATAAGGTATGGGTATGTGAGCTGT | |
| | GTGTGCTGCCCTTTACTAAGTCCTTATGGTAGATGTCTGAA | |
| | T | |
| AS27 | CCTCCTGAGATTTTGTCTAATGAAAGCTTGCATGGAGGAAT | 168 |
| | TTGGCCCCTAAGATTTTGGCTTCCCCATTGGATCATCAGTAT | |
| | GGAACGCAGTCAGTATGGAACTCAAACAAGAAAGACAGTG | |
| | TATGAAAAT | |
| AS28 | GAAGCCATGGATGGCCGGGAACCTCATCTGTTAGACAAAA | 170 |
| | ATGACGACAGATCCATTTCCTGG | |
| AS29 | TCTCTGCTCATTGGGGCGTTGAGGATAACGTGGAAGCGGG | 172 |
| | AAGCTGGGAGAAAGTCATTTTTTAAATGTGGATTGAGAGTT | |
| | CATATGTGGAAAGGGCTGCAATTCATTGAAATAACCAGGA | |
| | GAAATTGGATGCTGATATTTCAGAGCTGCAGTCTGTTTGGA | |
| | ACCTCACAGTACCGCAGTCTAACAAAGAAGTGCCAGCTTTG | |
| | TTTTAGATTTAAAAGAGTATCATCTAAAGGATCTGACATTA | |
| | CAGACAAAAGAGAGGTAACTCAC | |
| AS30 | ACCGTCCCCGCCAGTCAGAAGTTGAGCTGCTCACTGGACCC | 174 |
| | GAAACATCTCAGACAGTTTCACCTC | |
| AS31 | ATGGTCATGAGCTTTGTGAACCTGGCTCTGGCCAGGTTAAC | 176 |
| | TCAACTGCAGCAGGAGAGTCCAGCCCGAGTGTGGCCATCC | |
| | TGGGAAGAGGGCTCTTCAGTACGATTTTCCCCTCTAAACTG | |
| | CCGCCTTTTGCCCCCTAACTTGGAGTGCAGTTTTCCCACCG | |
| | AGACTTTTAGGGAATCCGACAGCGGCTGGGAACTT | |
| AS32 | TTGGAAGATCTGGATACCTGTATGAAACCTCATGCTTTTCA | 178 |
| | ACATGCCATGATGACATTTGCATCAACGACGTTTCGTTTAT | |
| | GTCAAAAAAGCTCCAAATTATCACTGCTTTCCAGT | |
| AS33 | TTTGGCACATCACATATGATGGTCAGACATTTT | 180 |
| AS34 | GGACATTTTGAAAGCATCAGGGCCGTATTATTTCATTGGAT | 182 |
| | TCCTGTAACTACACTGTTAGTCAGCCAAGTTATATATAAA | |
| AS35 | ATGCATACAGGAACTATTTCTCAGCATTGTCAGCTCCTGGA | 184 |
| | TTGCTCCTTGGGCGCTATACTGCACATTGGCTATACCAAG | |
| AS36 | GAAATTGAGGGTTCTTGCCATGGTGGTATGATCACTATGGA | 186 |
| | TATCAAAATGGGAAGTTACAGTTGCGATTACTTAAAACATT | |
| | CAGAAACATTATCTGCTATGGTGGTTAGCTGTGGAATATCC | |
| | TTTCTCTTTCTCTTTCCAAGGAAAAAAATTGCAAAATTGATT | |
| | TTTAAGGAACAAGTACCA | |
| AS37 | TGTGAAATCATGCCTCTGCAAAGTTATATCCTTCTC | 188 |
| AS38 | GTTAATAGAAAAAGTGTCAAAGGCTTCCACGTGGCTCATCT | 190 |
| | TCCTTCTGCC | |
| AS39 | TTCCAGGACAAGTACTTTATGATAGCTACGAAC | 192 |
| AS40 | GTAGCGCAGGTGGAAATAAGAGGACTAGAACCACCAGTGC | 194 |
| | ATGATCAA | |
| AS41 | GGCGGGGGAGGCGGCAGCGGAGAGCTAACAACACAGATC | 196 |
| | CCATGTAGTTGGAGAACCAAAGGCCACATACATGACAAAA | |
| | AGACTGAACCGTTCAGGTTACTTGCATGGAGTTGGTGCTTA | |
| | AATGTGAGT | |
| AS42 | GTAGCACCTTCACAGTCCAGGCAAGAGGAATGCTGGCGTC | 198 |
| | ATAAAATGAGTTGG | |
| AS43 | ATAGCCCTTCAGTACTGTTGTACGTGTTCCATTTCTCAA | 200 |
| AS44 | AGTGCTGTAGATGACATGATAGCAGCATTTCCCTTCAAAAG | 202 |
| | AGCT | |
| AS45 | ATGTGGACAAGGAAAGCAAGAGGAACTACCATTTGTGATA | 204 |
| | GACGAAGACAGATTTGCAGGTGGAGGGAG | |
| AS46 | ATTAACTACCGTCTGGGAATACTAGATAATGTTTCA | 206 |
| AS47 | ACTAGCACCAGCCCCAAATTTCCTGTTTTTATCTTTAGACTC | 208 |
| | GTGTGTTGGGTAAGGACAGAGCTGGTACTGCGT | |
| AS48 | ATGGCGATTCCGGGCAGGCAGAATATTGTTCTGAGAAGCC | 210 |
| | ACAGAAAAAGGGTAGGCATA | |
| AS49 | GAGGCTGAATTTGATAAGACCACTAGAACATTTCTACAAAT | 212 |
| | TTTCAATCGCAAAAGAGGAAAAATACAA | |
| AS50 | AAACTGCTGTATGAAGTTTCCCAGAAAATGGTCTTA | 214 |
| AS51 | TGGGCCTTCATCCATTCTTTGCCTGCACTACTGGGTATTTTC | 216 |
| | TCATACCCCCCAAGTCGA | |
| AS52 | TCAAGCCTACAGGAACATAGAAAGCTTCTCTCC | 218 |
| AS53 | CGATTTTCTCGGCGGTATCCATGCAGTGCTAGGACCAGCCT | 220 |
| | TGGGAGAGAGGAAAGTGAGCCACCAGGAGAAGCTGCCATG | |
| | TGGAGGTGGAGGCTCTGGAGGGGTCGGGCATCAGGACACC | |
| | CTTCAGGAGAGGGGAGACCTCCTGAGACAAACCAAGGGCA | |
| | TTCCACTGGGAGA | |
| AS54 | GAAAGAAGCCACTCCAAAAAGAAGGATGGTCTTGTGAGAA | 222 |
| | ATGACGGACCTGGATTCCCACCTATGTTTTCCACATCAGAA | |
| | AATACCAACTTGGAT | |
| AS55 | GCTGGTGATGTATTATTCATTCCTGGCATTAGTTTGAAAGT | 224 |
| | CCATAAAGTTCAACACAGCAAGGTAATTAACATCTGTAAT | |
| AS56 | ATCGAGAACGAAGGCGGCCAGGTGAGGTCCCGCCCCGCTC | 226 |
| | CCTCC | |
| AS57 | CTCATCAGCGTGGTCCAGAATGGTAAGGAAAGCCCTTCACT | 228 |
| | CAGGGAAGAACAGAAGGGGAGATTTTCTTTGATGGTTGTTT | |
| | GGAAGTCAGGCTTAAACAATTGTGTCTGTGTGTGCGCATGC | |
| | ACAAACACTTTTACCTTATCTTTATTTTCTTCTTTTTATTTGA | |
| | ATGTA | |
| AS58 | GAACATCAAAGAAATAGCAGTAAGGTATTTTATGGTGTTAT | 230 |
| | TGACTGTGTCATAAAGGAAACTATTACTTGGCACGAT | |
| AS59 | GACTCGCTTCCCAAAACTGAAATGGTGAGTGGGTCATACA | 232 |
| | GAATGGGTCTGTGTGAGCACTGTCTTAATCAA | |
| AS60 | CGCCCGCGGAAAGACACCTGGAAGGTTAGAGATCCAGCAT | 234 |
| | TGCGCTACACCCCTTTGTTAATTCAGTCACTGGACAGCCGC | |
| | CTAGCCGAGAGCTGTGCGGTTTTTATATGGTATTGTATCTTT | |
| | ACTTTAGGCGATACATGCAGAAGTCGTCCGGTAGAAAAC | |
| AS61 | CCCGTGGGCAGTGTCAGCTTCCAGGTAGGGTGTGTGCAGCT | 236 |
| | TTTCCCT | |
| AS62 | CAGAGGATTCAGGCACAGGAAGGTAAGTGTCCTGTAAATC | 238 |
| | TCTCCCAGCCCCTT | |
| AS63 | AACCTCAAGCCAGACAGAAAAGGTAAGGAGAGCATGGCA | 240 |
| | AAG | |
| AS64 | GACCTGAACCAAGATGGATACAATGGTAATTTAAACCAAA | 242 |
| | GCGTGGAAGTCATTTATGTGTCGTCACCACTGCATAAAGGA | |
| | GACCATGCCTGTTTTAAAGTATTCAGTATGGTGTGTGTGGG | |
| | TGCAGTTATTATTACTGATTTT | |
| AS65 | GCGCCACATCTCCTGAGATTATTTGGTAATATGTCATGTAG | 244 |
| | AAAA | |
| AS66 | TCCCTGAAGAAAGTTGTGAAGGAGGTGGGTGTTGAGAGTT | 246 |
| | GGAGAGGGAGAGGTGCGTGGAGGGGAGCC | |
| AS67 | CCAGTAATCCTGTTCAAGGGCAAGGTAAGGCCCATACCA | 248 |
| AS68 | CAAATATTAGAGCAACCTAAACAGGTAAGATTAAAGGGGT | 250 |
| | GGGACTTTAAATGT | |
| AS69 | ATAGCAACTGGAGCTCAGGTAGTAACACAGGATACTAATT | 252 |
| | ATTTCTTATTTTGCTTTGAAAAATTTGTTCCAGTCCAC | |
| AS70 | CTGCTGTATGAAGTTTCCCAGGTGAGTGCAGGGTCTGATAC | 254 |
| | CAGCAAC | |
| AS71 | TGGAAGCTAAAGGCCACTAAGGTAAACGGCACTCCTGATG | 256 |
| | CTTATGGAGAGAGTATGAGGGCCTTGTGGGGAGGGTCTGC | |
| | ATTCCAACTTGGGGGTTTCCAG | |
| AS72 | AGTCCTGCAATTTATAAACTAAAGGTAAACATACAAAACA | 258 |
| | TAAAGGGAGAAAACTTAAGACATAACCATTTC | |
| AS73 | GATATGGAGGTTTTCCTCAAAGAGGCAAGTACTAACCAAC | 260 |
| | ACAACACTTTGATTCACTTGACACTTACTTTCAGCACT | |
| AS74 | CTTCAAGCGAATCAGCTACAAGGGGTAAGTAAAGGAAACC | 262 |
| | CAGTTTGT | |
| AS75 | TTAACAGATGATCAGGTTTCAGGTAAGTTGGTTTCCAACTC | 264 |
| | CTTTACACCCTTCGTTTCCTTCATCTTTCCGTTCTTCCCTTTC | |
| | CTTCTTTCTTTAATTGAAGAA | |
| AS76 | CAAAGAGACAGAGACTCCATCAAGGTAGGACTCAAAATTT | 266 |
| | ACAGACTAAGGAATGGCTTAAAAGGGCTG | |
| AS77 | AAGCGCTATGGCTGCGTTTTTCAGGTCAGTATCCGACATTT | 268 |
| | GTCCTTCCCAGTCACTGACATTCTGCCA | |
| AS78 | GAAGGGACACCTTTTGAAGATGGTAAGTCATACTCATTATG | 270 |
| | TTTTCTA | |
| AS79 | AGTATGACCCAAGCCCTTTGCAGGTACTTCTTCATGACACT | 272 |
| | ATTAATGGTAATTGTAGATTTGGGGAAGAGGAAGTATTTTG | |
| | TATTTTGTGAG | |
| AS80 | TTACTGGATGGAATGATATTCCAGGTAAGTAGGTCTGTATC | 274 |
| | AGACTGTAACAGTGGGGCTCATATTAGACGACAAAGCTCA | |
| | GAATTA | |
| AS81 | GAAATACCAAGGAAGACTATTTTTGGTAAGAACCAGGTAG | 276 |
| | ATATGAGGCCA | |
| AS82 | TCTCAGAAACAACCAGCTGAGAAGTGTCTTCTCAGAAACA | 278 |
| | ACCAGCTGAGAAGGCTACAAGTGACGAGAAAGATTCTGTT | |
| | TCAAATA | |
| AS83 | TCTCGGAAAAAACCAGCCTTGAAGTGTCTTCTCCGAAACAA | 280 |
| | CCAGCAT | |
| AS84 | TCTCAGAAACAACCAGCCTTGAAGTGTCTTCTCAGAAACAA | 282 |
| | CCGGCCT | |
| AS85 | TCTGAGCAACCACCAGGCTTGAAGTGTCTTCTCGGAAAAAA | 284 |
| | GCAGCCT | |
| AS86 | GTGTACTACCTCTACAAGAACGTGGCCTATAAGGGCTCTGA | 286 |
| | TATAGAT | |
| AS87 | CAGCTGAGCGTGGTGACAAGGGATGATTACAACAGCAGTG | 288 |
| | AATTGAAA | |
| AS88 | CTGGAAGATAAAGAACTGGGAAGGATATCTGTATGCCCTT | 290 |
| | GGACAGAAGGTTTGGAAAAGATGGAAAAAACGTTACTTTG | |
| | TTCTAGTTCAGGT | |
| AS89 | GAAAATCAAAGGCTCAGTAAAAAGGATTTGGTGAGTGAAA | 292 |
| | AGTTGAAG | |
| AS90 | ATGTCGGCGACTACAGGTTCCCAAGACAACCCAGAAGGGA | 294 |
| | AAAGGGACCCGTCAAGGAAGTTCCAGGAACAAAAGGCTCT | |
| | CCCTAAAAGACCACCGCTTCAAAAAAACCTGAGGAATGGA | |
| | GTGGGCCAACACTATCCAGCCACTCTGACCAGCCGAACGA | |
| | GGAACTCAATCAAAATGCGCCATAGCAGGACCACAAGGGC | |
| | AAGGAGACCACCGCCTTCTCCAGTGCTTCCTTGGGCAGCCA | |
| | GTAATTCCCAGGCAAGGCCAGAGACTTCAAGTCTATCTGAA | |
| | AAGTCTCCAGAAGTC | |
| AS91 | CAGGACCTCGCCACAGAGGATACCGGAAGTGTGACAAAA | 296 |
| AS92 | GATCCCACTACGAAACGCTCCAGATTGACCCCAAAGTTGCA | 298 |
| | TTTCCTCGTCGAGCGCAACCCAAGATGGTCACAAGAACAA | |
| | AGAAAATATTTG | |
| AS93 | ATATTGGCTCAGAAGAAGAAGGATGAGCTGAGCAGC | 300 |
| AS94 | GCTAAGTTTTGGAGTGATATTTCAATTAACCGAGCCCCAAG | 302 |
| | CTTTGGA | |
| AS95 | CTGGATCCACCTCATGATGAAAAGGCTCTGGCATGCCCTCA | 304 |
| | ATGGACATTATTGTGGATATTTTGGACAGCGAAGGCTCTT | |
| AS96 | AAGAAAGAAGATGTTAACTTTCAGGTAAAACAGTTAATTC | 306 |
| | AC | |
| AS97 | GGAAATGTATTGGTAAGATATGTAAGTTTATATGTTTACTA | 308 |
| | TATTCTACTGAAC | |
| AS98 | CAAAGAAGTTTCATCAAAGCAGCAAGTAAGTCTTTTTTGTC | 310 |
| | TCAGTTT | |
| AS99 | TTCTCGGGTCGTTCAAGAAGCCGAGGTAAGACACGCCGCG | 312 |
| | ACGGGTTTCTGCGGGCTACCAGGAACAAT | |
| AS100 | CGAGAAAGTAAGAGGCACCAGGGTAAACGATGGACTCTCT | 314 |
| | TTCCTCATCGT | |
| AS101 | CACCTGGCCATCCCCGCCAAGATCAGTGAGTGCCGGAGCC | 316 |
| | CAGCCCAGTCCCGACTACCCCGCCAGCGAGACCCCGGGGC | |
| | AGGCCGGTCACCTGGCTTCTCCTCCTGCCCGCAGCCCGGGA | |
| | GAAGCTGGACCAAGTGGCGACAGCAGTGTACCAGA | |
| AS102 | TTAGACCTGGCCAATCCTCAAGGTAAGGGCCCTAAGGGAG | 318 |
| | AACTGAGGGACTTCGCACCAAGGACAGAAGAAGCCCCGGT | |
| | CTGCCCTGCGCTGCCA | |
| AS103 | AGGGACCTCCGCAGCATCGTGGAGGTAAGGCGGGCAGACA | 320 |
| | CCAGAGGGCAGTGGGTACCCAGGCCAGCCCCTTGGCCCCC | |
| | A | |
| AS104 | CTGCTCACAGAGATGGACAACAAGGTGAGGCAGGGGCATG | 322 |
| | GCGGGGCTCTCCATCCCACAGCCTTGCCCCCAGGATCGGGA | |
| | CAGCACCACCTTCTCCTGGGTCAGCCCCCTACCTGCTTCCTT | |
| | AGAGGTCCCCTGGCCCCTCTTTCCCCGCCACAGCTGGACCG | |
| | CCTGGAGCTGGACCCA | |
| AS105 | ATGCTCAGAAGGTGCCCATGTGTGACAAATGTGGTCCTGGC | 324 |
| | ATTG | |
| AS106 | GCAGGAAAGGAGCGTAGCACCCAGGTAAGGGTGGGTCCCA | 326 |
| | CTCTG | |
| AS107 | GCGGGGCGAAGTCTGTGTCTCACGGTCAGTTCAACTCCAGG | 328 |
| | CTCCTGGGGAGCGTCTGGGTTCCGGCGACTAGGACGCCTAA | |
| | CTTCCTTTAACTTCGGGTCTGAAAGTCTGTGGCTCTTTTACA | |
| | GAGAAGCAGCGTTCCAGGGACTCCGTTCCCGCCACCCGTTC | |
| | AGCAACCCTCTACACCTGGACCCGACCTCCTCGCCTTGGAG | |
| | GAAGAATATAAGCGTT | |
| AS108 | GTTGAAGATGAGGAGAAGAAAGAGGCAGGGACTCATTTCA | 330 |
| | TCCACCTGACTGGAACCACTGTCTCAGCTGGAGTCCCTGAG | |
| | GAGATGCCAGCCACAACTCTCCGAAGAGAAGTATTC | |
| AS109 | ACTGCCATCCTCTTCACCAGGCAGCAAAGCAAGGCCCTCCC | 332 |
| | ATCCTTGGGGATTACAAACAGACATGGATTTTGGGCTGGAC | |
| | AAGGAAATACAGTGATGCATGGCAGAGATTCGGATGGGAA | |
| | ACAAGTTAAAGGTACAGTAGTAATACTAGCGCAGGGTGCT | |
| | TTTCACCAG | |
| AS110 | AATGCTTCACTACATGAAGTCAAAGATTTCTTTGTTCCTTTC | 334 |
| | CCACTCCCACTACTTCATTTGACTAGCCTTAAAAGAAATAA | |
| | ATTATTTAAT | |
| AS111 | GGTACTACCCGTCTTCTATCTGGGGAGGACTTTTCC | 336 |
| AS112 | TCTGAAATGGTGGCACATGAAAGGTATGTTTTTGAAGCTAC | 338 |
| | CGGAAATGATTCTTTTAGAACTCTTTGTTTATTGATGAAA | |
| AS113 | ATAATTATGGCATTAGCCATCCTGGACCTCTTTGCAGTCTT | 340 |
| | AAAAATTGAAGCCCACAAACAGCTCTTACTTAAGAGTTTCT | |
| | GCTATGGTGCTGGAAAA | |
| AS114 | CAAATTAGGGTACCATCATATTCAGATGAAGGC | 342 |
| AS115 | CGGCAGAGGGAGGTGGTGGACCTGGTGAAGAAGGACCTGG | 344 |
| | TTATGCGTGCAGTGAGTCTTGGAACTCAGAGGGGAGACCA | |
| | AAATGGGAGGCCATCGTGGAGCGGACATGCGATATGGGCC | |
| | CGTATAATGGAATGTGCTTACAAGGGCCAGCAGGAGTGCC | |
| | TGGTCGAGACGGGAGCCCTGGGGCCAATGGCATTCCGGGT | |
| | ACACCTGGGATCCCAGGTCGGGATGGATTCAAAGGAGAAA | |
| | AGGGGGAATGTC | |
| AS116 | GTGCACTTGGATTTTTCTTCAGCAGAGATGGGATTTCCTCA | 346 |
| | TGCTGCCCAGGCTAATGTTGAACTCCTGGGCTCAAGTGATC | |
| | TGCTCACC | |
| AS117 | GGAGAAAAGATCCTCTTCATTAGGGGTCTTCATGGTTGTGA | 348 |
| | CTATATCTCCACAGCCAAAGCAAAGCAGACATTTAAGATG | |
| | CTGCACAACAAACATGTCATG | |
| AS118 | GGCTTCTTTGAGACTGAAATGGCAGTCCCCTTCTTTTCCAG | 350 |
| | AAAAATTAGAAAAGTCAGAAGAGCAATAATGAAATGGACA | |
| | TTTTGCTGTTATTTCTAT | |
| AS119 | GACAGTCAAGTGGAAACAACTGTCTGGGGAGTTGGAAATA | 352 |
| | GGCAAGAATGGAGAGAAATTTTGTATGGCAGTACACAGAA | |
| | TTTTGCAAGTTTATTG | |
| AS120 | TGGACTCGACCAGAGAAGCAAGAGCTCAACTCATCTTGTTG | 354 |
| | CTCACTTATTTTA | |
| AS121 | CTGGGTCGCTTGGAAGAAGCCAAGTCCTTTGAAACTGAGG | 356 |
| | AAAAC | |
| AS122 | CTGGGTCGCTTGGAAGAAGCCAAGGTGCCATTGAAAATGC | 358 |
| | TGCTCTTC | |
| AS123 | TGGCTCCTGCGCACCTGGGAGAGAGCTGACAGTGGCCTT | 360 |
| AS124 | CTTGTTGCAAGGCCAGAGATTCAGATCTTTATGGAAGAAGA | 362 |
| | TGTCATGAAAGTAGAG | |
| AS125 | AGACACCAGGAAAATGTCCACACTGTATTAGTACATGGAA | 364 |
| | AAGTGAAAGGCCTGTTTTATATA | |
| AS126 | ATCATGCTCATTGGGAATAAGAGCAACTGCCAGAAACAGC | 366 |
| | TTCTCAGAGATATGAAGATATTGAACGAATTTTATCATGGT | |
| | CTGATATTGACATTGCAACTGTCT | |
| AS127 | CCAAAAGTGTTTCCCAAAAATCAGGTAATATCTTTAATTCC | 368 |
| | AAAAGCACAGGAAGACCCTGAGCATACT | |
| AS128 | GGCTGGAAGATTTGCCGGGAAGGCATTGGGCCTGTTATGCT | 370 |
| | CACAATGACTTCAGGTGAATGGAGTTTCAGTTTCCTGGAGG | |
| | CCATCTGTGGAGTTTCCAGGGAACCTGTACCGAGGAGAAG | |
| | GCATTGTATATGGGACACTAGAGGAGGTGTGGGACTGTGT | |
| | GAAGCCAGCTGTTGGAGGCCTACGAGTGAAGTGGGA | |
| AS129 | ATTGATCCAAGCTCTCCCTTACATACCTACTATGAAAGAAT | 372 |
| | TACTAAAGGACGTAATCCAGAAAGAAGATATATGAAACCG | |
| | GAACGAATCAGTCCGGGACACGAGGCCATGGAAAAAAACC | |
| | TTGAAGATAACTTA | |
| AS130 | AACATGGCTGACATGCTTTATAATTTCCTTTCACAGACCTC | 374 |
| | AGCAGCTGCCAAGTTGAATCTGCAATTTGAGATGATAACA | |
| | GCAGTATTCAGGCTCAATAGATGTGCGGAATGTCTAGGGCT | |
| | ACTTCTCCAGGAGAACAGC | |
| AS131 | CAGATACTACAGCAAGCCTTGAAGAAACTGCCTTTTTCATT | 376 |
| | ATGTATTACTGAGTGTGAGACCATAGCCTATCACCTTGCCA | |
| | GGGATAGTAATGGAAACTTGGAATTA | |
| AS132 | CAGATACTACAGCAAGCCTTGAAGATAGCATTTTTGAAAGT | 378 |
| | GGTGGTGCCTTTGAAACTGCATCCTTACTCCTTTTGTCAC | |
| AS133 | TATGAGAGTCCCATCTTCAGCAGAGACCGAGTGAATCCAG | 380 |
| | GTGCCCTGAGTGGCTCGCTGGGTCAGGCCCGCGCGGCCTGC | |
| | TGGAGGACCTTGGGGTCTTTGAAGCCTGGGGGAGAGTTCA | |
| | GAGCGGCAGAACAGCGCAGGGCTGGCTGGGTGCCAGGCGC | |
| | TCCCGGGTTCACCTGCAGTTTAATAAGCTCGAGGTTTCCGA | |
| | GAGAGCGCTTTTGCCTACGCCATCGCAGCAGCTGGCGTGGT | |
| | GCACGCCGTGTCCAATGCGTGTGCCCTGGGCAAACTGAAG | |
| | GCCTGTGGCTG | |
| AS134 | TTTTCTCAGAATATGAATACGAAAATGATTAGAGGAACAG | 382 |
| | AAAGAAAGCAACAGTACTATGGACTACAAATTTTGGAA | |
| AS135 | GGGGAGCCAGGGGAGGACGCAGGAATAGGGAGGCCTGAG | 384 |
| | GAGAGGGAGAGAGAGGAGGAGATGGCTGATCAG | |
| AS136 | CCTCCGGGTGGACATTCCCCTCAGGATTTGTCTATTAGAGA | 386 |
| | GATCTTTTGCCTTGGGCATAAAGAAGTCTGC | |
| AS137 | AACTACAGAAACCTGGTCTTCCTGGATCCCTGTTTTCAAGA | 388 |
| | AAATCTT | |
| AS138 | ACTCAACGGTTGCCAGGACACAAAGTAAGCAACAGCTGCA | 422 |
| | TGACCGGTTTAGTCCTGACGTTTACAAAGAGGGACCCTTTC | |
| | CATAAGCCTGTAACTTGGTGTGGGCAGCTTGCCGATGTCAG | |
| | GCAGTGCATGTTTCACTCGATTAGGGAGAGAGCGCACCCTC | |
| | TCCAGAGGGCTTTGGCCACGCTTAATTTTTTCTTTGTTTCCT | |
| | TCTATACTGCTTTATATCTCACACATCCCCTCTTAACTCTCC | |
| | AGACATGGGAAGTTGTTGTGACAGGTCAGGAAAGTCGTAT | |
| | GTTTACCCTTCTCCTAGAAATTAGTTATGTAAGCTATTATTG | |
| | TATGTATTTAGTAATGAGGGGACATGTGCAT | |

Table 11 shows gene origin, full gene name, mutation and amino acid sequence of identified neoantigens that arose from point mutations events. Point mutations are indicated with bolded letters. Table 9 shows their corresponding polynucleotide sequences.

TABLE 11

| Neo- | | | | | SEQ |
|---|---|---|---|---|---|
| epitope | | | | | ID |
| ID | Gene | Full Gene Name | Mutation | Protein | NO |
| M1 | KRAS | GTPase KRas | G12A | YKLVVVGAAGVGKSALT | 389 |
| M2 | KRAS | GTPase KRas | G12D | YKLVVVGADGVGKSALT | 391 |
| M3 | KRAS | GTPase KRas | G13D | KLVVVGAGDVGKSALTI | 393 |
| M4 | KRAS | GTPase KRas | G13R | KLVVVGAGRVGKSALTI | 395 |
| M5 | KRAS | GTPase KRas | Q61H | LDILDTAGHEEYSAMRD | 397 |
| M6 | KRAS | GTPase KRas | Q61R | LDILDTAGREEYSAMRD | 399 |
| M7 | KRAS | GTPase KRas | Q61K | LDILDTAGKEEYSAMRD | 401 |
| M8 | DIS3 | Exosome | R780K | YTHFTSPIKRYADVIVH | 403 |
| | | complex | | | |
| | | exonuclease | | | |
| | | RRP44 | | | |
| M9 | BRAF | Serine/threonine- | V600E | IGDFGLATEKSRWSGSH | 405 |
| | | protein kinase B- | | | |
| | | raf | | | |
| M10 | BRAF | Serine/threonine- | G469A | QRIGSGSFATVYKGKWH | 407 |
| | | protein kinase B- | | | |
| | | raf | | | |

TABLE 12

| Neo- | | SEQ |
|---|---|---|
| epitope | | ID |
| ID | Polynucleotide sequence | NO: |
| M1 | TATAAACTTGTGGTAGTTGGAGCTGC | 390 |
| | TGGCGTAGGCAAGAGTGCCTTGACG | |
| M2 | TATAAACTTGTGGTAGTTGGAGCTGA | 392 |
| | TGGCGTAGGCAAGAGTGCCTTGACG | |
| M3 | AAACTTGTGGTAGTTGGAGCTGGTGA | 394 |
| | CGTAGGCAAGAGTGCCTTGACGATA | |
| M4 | AAACTTGTGGTAGTTGGAGCTGGTCG | 396 |
| | CGTAGGCAAGAGTGCCTTGACGATA | |
| M5 | TTGGATATTCTCGACACAGCAGGTCA | 398 |
| | CGAGGAGTACAGTGCAATGAGGGAC | |
| M6 | TTGGATATTCTCGACACAGCAGGTCG | 400 |
| | AGAGGAGTACAGTGCAATGAGGGAC | |
| M7 | TTGGATATTCTCGACACAGCAGGTAA | 402 |
| | AGAGGAGTACAGTGCAATGAGGGAC | |
| M8 | TACACACATTTTACTTCACCCATTAA | 404 |
| | AAGATACGCAGATGTCATTGTTCAT | |
| M9 | ATAGGTGATTTTGGTCTAGCTACAGA | 406 |
| | GAAATCTCGATGGAGTGGGTCCCAT | |
| M10 | CAAAGAATTGGATCTGGATCATTTGC | 408 |
| | AACAGTCTACAAGGGAAAGTGGCAT | |

### Example 2: Quantitative PCR Analysis of Multiple Myeloma Neoantigens in Tumor and Normal Tissues

Multiple Myeloma (MM) neoantigen candidates were tested for their expression in following samples:
20 CD138+ plasma cells derived from MM patients
20 MM and lymphoma cell lines (NALM6, Daudi, MM1R, MOLP8, JIM3, ELM, H929, OPM2, RPMI8226, MM.1S, KMS11, ARH77, IM9, JIM1, KMS12-BM, MOP2, HUNS1, U266B1 and HTK)
11 PBMCs obtained from healthy donors, 5 from young (<30 years old) and 6 from old donors (>60 years old)
Sorted immune cells derived from 3 healthy donors (B-cells, Plasma Cells, T-cells, PBMCs and monocytes) and
18 healthy donor derived tissues (liver, kidney, pancreas, prostate, mammary gland, colon, stomach, skeletal muscle, lung, ovary, placenta, small intestine, spinal cord, uterus, spleen, brain, heart and bladder)

Quantitative PCR primers were designed to span the breakpoint junction sequences using the Primer Express software (version 3.0.1). Primers with Tm of 60° C., GC content between 30-80% and low likelihood of forming stable secondary structures were selected for expression analysis.

RNA from these samples was isolated using Qiagen RNA isolation kit (#430098094) as per manufacturer's protocol. Complementary DNA libraires were prepared using oligo dT primers provided in the high-capacity cDNA reverse transcription kit (Invitrogen-part #11904018) from 200 ng of total RNA. Next, 3-10ng of cDNA was pre-amplified for 10 PCR cycles in 15 ul ofpre-amplification mix using TaqMan preamplification kit (ThermoFisher Scientific, #4384267). For each sample, input cDNA was estimated to keep the Ct values of endogenous controls (RPL13A, GAPDH, HPRT1, B2M) in the range of 13-15 Ct values. Among the tested control genes, RPL13A showed the most consistent expression among the healthy tissues. Finally, the pre-amplified cDNA was diluted 5 folds and loaded onto Fluidigm Biomark™ HD for 40 cycles of PCR amplification.

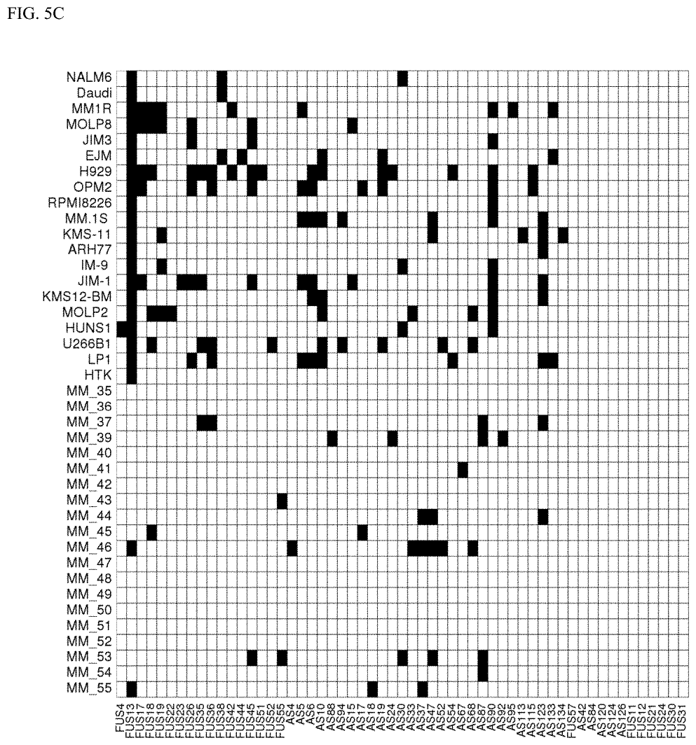

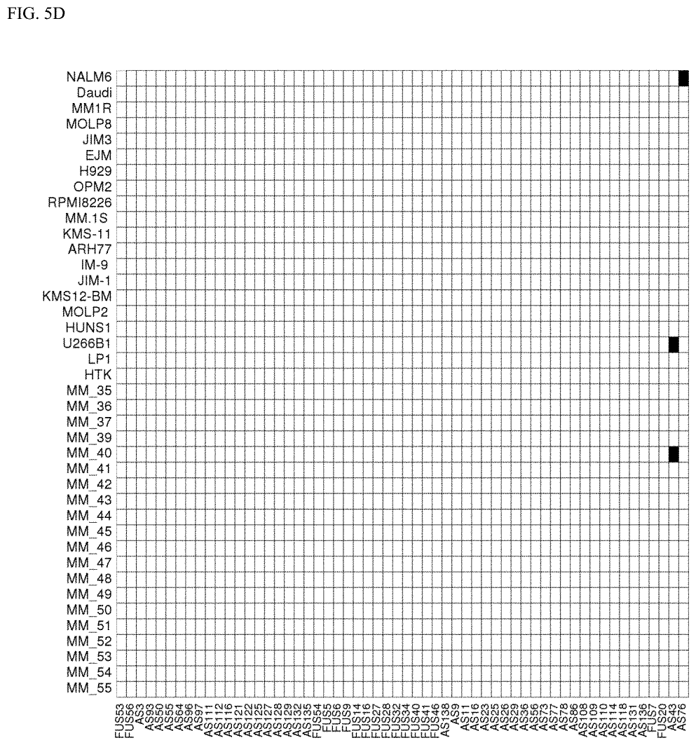

The expression of the neoantigen candidates (Ct values) was normalized against an endogenous control, RPL13A. A cutoff value of ACt<15 (fold change of ˜32,000) was used to determine the expression of neoantigen candidates in a biological sample. The results of the expression profile for all the tumor restricted neoantigen candidates are shown in FIG. 5A, FIG. 5B, FIG, 5C and FIG. 5D. Antigens with expression in both control and tumor samples are shown in FIG. 6A, FIG. 6B, FIG. 6C and FIG. 6D.

### Example 3: In Vitro Immunogenicity Assessment of Neoantigens

The immunogenecity of neoantigens was assessed using known methods. The 9 mer fragments selected in Example 2 were assessed for their ability to activate T cells using known assays, using TNFα and IFNγ production by CD8⁺ T and CD4⁺ T cells as a readout. Peptides were synthesized by GenScript with purity >80%. The lyophilized peptides were solubilized in 100% DMSO.

### Exogenous Autologous Healthy Donor Restimulation Assay

Overlapping 15-mer peptides were designed to span each indicated neoantigen. Their ability to activate T cells was assessed using known methods, such as the assay defnied as exogenous autologous normal donor restimulation assay. The peptides were used as pools using TNFα and IFNγ production by CD8⁺ and CD4⁺ T cells as a readout. Maximum frequency of CD8⁺TNFα⁺IFNγ⁺ and CD4⁺TNFα⁺IFNγ⁺ T cells and maximum fold change over background for each pool of peptides were analyzed, which were calculated as the highest frequency of CD8⁺ TNFα⁺IFNγ⁺ and CD4⁺ TNFα⁺IFNγ⁺ T cells and resulting fold change across the normal donors evaluated for the peptide.

CD1c+Dendritic Cells (CD1c+DC) isolated from human healthy PBMCs were thawed using media (IMDM (Gibco) supplemented with glutamine, HEPES, 5% human serum (Sigma), and 1× Pen-Strep). DC cells were resuspended in media supplemented with IL-4 (Peprotech, 20 ng/mL) and GM-CSF (Gibco, 20 ng/mL), plated in 6 well microplates, and rested overnight at 37° C. and 5% CO2 incubator. The following day, DC cells were counted and plated in a 96 well round bottom microplate at a concentration of 30,000 viable cells per well. Lyophilized neoantigen peptide pools (15-mer peptides with 8-mer overlapping peptide sequences) were solubilized in 100% DMSO with a stock concentration of 20 mg/mL Neoantigen peptides pools were added to DCs for a final concentration of 10 μg/mL and rested for 2 hours at 37° C. and 5% CO2 incubator. CEF Peptide Pool “Plus” (Cellular Technologies, Ltd.) was utilized as a positive control (each viral peptide at a final concentration of 4 ug/ml) and DMSO at the same final concentration (0.05%) as the experimental peptides was utilized as a negative control. After 2 hours, DC cells were irradiated with 50 gray of ionizing radiation. Autologous CD3+ Pan-T cells isolated from human normal PBMCs were thawed using media. Following irradiation, autologous Pan-T cells were added to the irradiated DCs at 300,000 viable cells per well. Human IL-15 (Peprotech) was added to all wells at final concentration of 10 ng/ml. Plates were incubated at 37° C. and 5% CO2 incubator for a total of 12 days. Media was refreshed every 2-3 days with IL-15 (R&D System, 10 ng/mL final concentration) and IL-2 (R&D systems, 10 IU/mL final concentration).

On Day 11, cells were re-stimulated with identical experimental peptide pools or controls, at same concentration as peptide stimulation on Day 1. Protein Inhibitor Cocktail (eBioscience) was added to every well and plate was incubated overnight for 14-16 hours at 37° C. and 5% CO2 incubator. On Day 12, cells were stained for surface and intracellular flow cytometry analysis. The cells were washed with PBS and stained with Live/Dead Fixable Aqua Dead Cell stain (Thermo-Fisher). Following the live/dead stain, cells were blocked using Biotin-Free Fc Receptor Blocker (Accurate Chemical & Scientific Corp). Extracellular cellular flow panel (1 μL/antibody per well in 50 μL) consisted of CD3 PerCP-Cy5.5 (Biolegend), CD4 BV421 (Biolegend), and CD8 APC-Cy7 (Biolegend). After extracellular staining, cells were fixed and permeabilized using Foxp3/Transcription Factor Staining Buffer Set (eBioscience) and stained for intracellular proteins (1:50 dilution) using TNFα FITC (R&D Systems) and IFNγ BV785 (Biolegend). Cells were washed and resuspended in stain buffer, analyzed, and recorded in a BD Celesta flow cytometer.

Flow cytometry analysis was conducted on FlowJo v10.6 software. Cells were gated on live, singlet, CD3+, CD4+ and CD8+ T cells. The CD8+ and CD4+ T cells were analyzed for TNFα and IFNγ expression.

Immunogenicity responses were considered as positive for a peptide pool if the following criteria was met:
Frequency of double positive TNFα/IFNγ CD8+ and/or TNFα/IFNγ CD4+ T cells upon stimulation with an experimental peptide pool was greater than or equal to 3-fold over the DMSO control
Frequency of double positive TNFα/IFNγ CD8+ and/or double positive TNFα/IFNγ CD4+ T cells was at least 0.01%

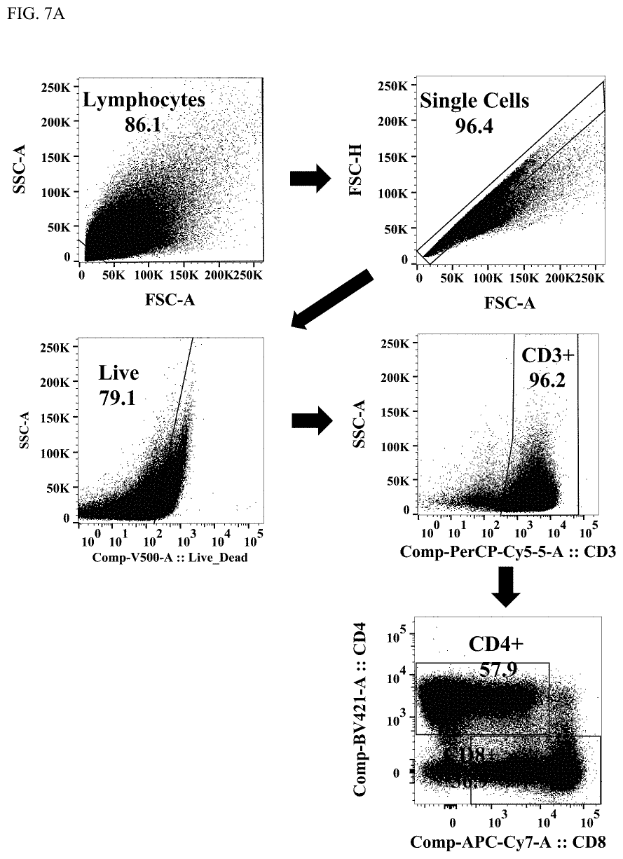

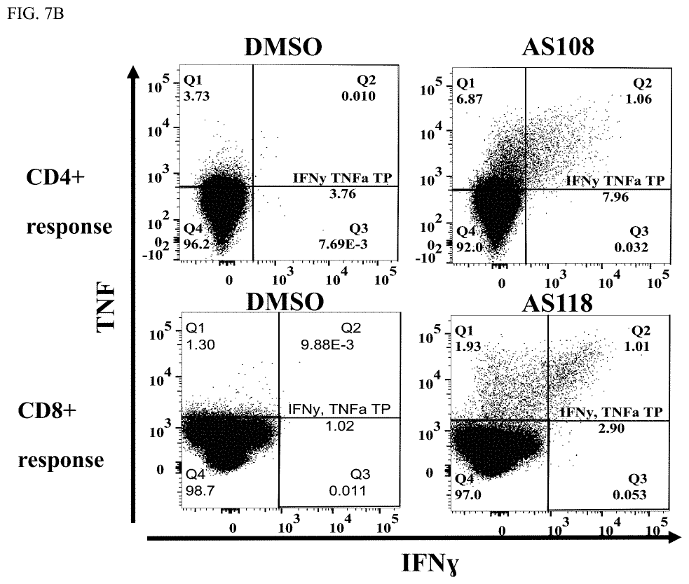

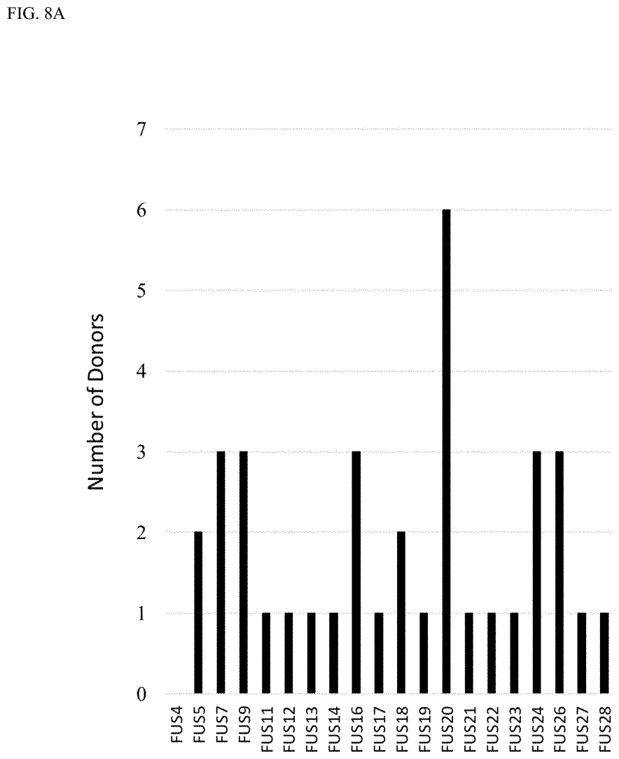

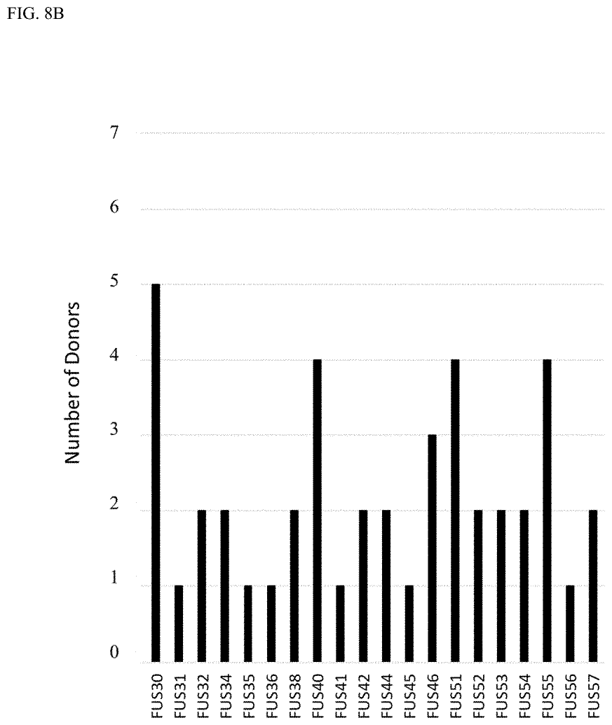

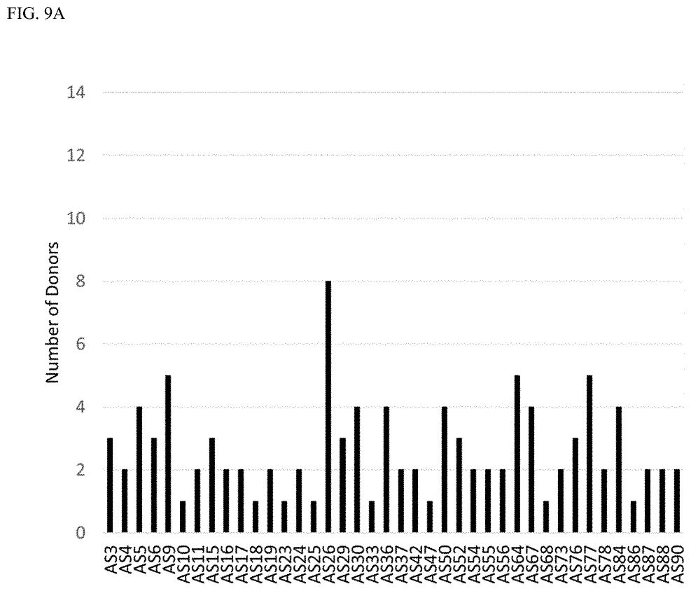

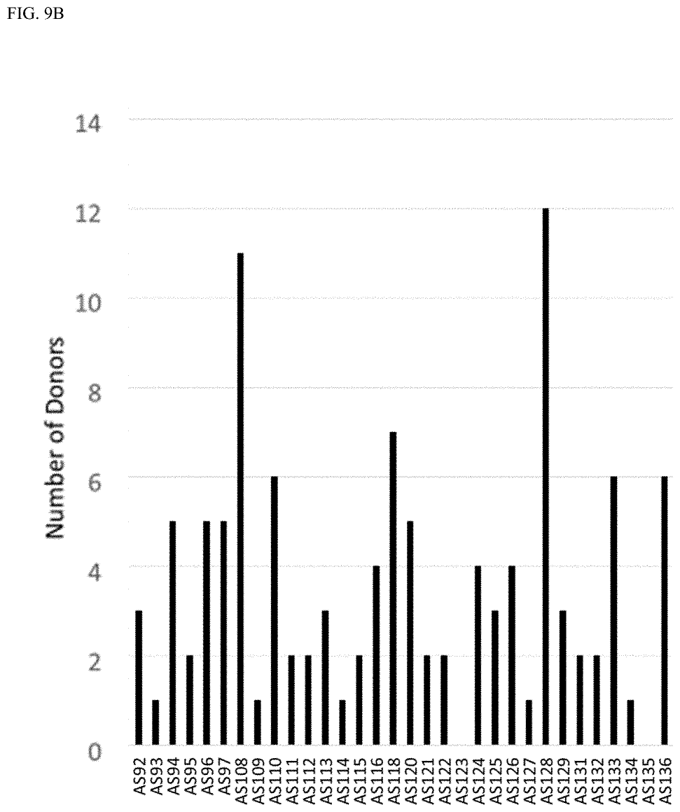

The immunogenicity of neoantigens was first investigated in 5-7 healthy donors. The non-reactive neoantigens were further tested on a new cohort of 22 healthy donors. The immunogenicity data for the neoantigens is summarized in Table 13. FIG. 7A and FIG. 7B displays a representative dot plot showing the gating strategy and the immunogenic responses achieved for few neoantigens. Interestingly, majority of the neoantigens showed immunogenic responses in multiple donors (FIG. 8A, FIG. 8B, FIG. 9A and FIG. 9B).

TABLE 13
Immunogenicity data summarization of all tumor specific neoantigens.
For each neoantigen, the maximum CD8+ and CD4+ T-cell
responses (TNFα and IFNγ) are reported. The responses reported
are donor independent.

| | | | | Fold | Frequency | Fold | Frequency | |
|---|---|---|---|---|---|---|---|---|
| | | | | change | (TNFα + | change | (TNFα+ | |
| Neo- | | | | (CD8+) | IFNγ + | (CD4+) | IFNγ+ | Immuno- |
| peptide | SEQ | | Neoantigen | vs. | CD8 T | vs. | CD4 T | genic |
| ID | ID | Gene ID | Sequence | DMSO | cells) | DMSO | cells) | (yes/no) |
| FUS4 | 7 | CD5L-> | DVAVICSGR | 3.1 | 0.04 | 7.13 | 0.029 | No |
| | | FCRL1 | RSARDP | | | | | |
| FUS5 | 9 | CD5L-> | DVAVICSGV | 4.59 | 0.079 | 6.97 | 0.021 | Yes |
| | | FCRL1 | PVADVS | | | | | |
| FUS6 | 11 | CD5L-> | EDVAVICSE | NA | NA | NA | NA | NA |
| | | FCRL1 | LFLIASP | | | | | |
| FUS7 | 13 | FAM98A-> | GRAGQGGGMI | 3.04 | 0.093 | 17.18 | 0.21 | Yes |
| | | LOC105374454 | SVSQEFIK | | | | | |
| FUS9 | 17 | EAF2-> | SGLLMNTLT | 5.53 | 0.13 | 3.08 | 0.031 | Yes |
| | | SLC15A2 | VLILYFLY | | | | | |
| FUS11 | 21 | GAB1-> | GKSTPPRKEI | 7.36 | 0.013 | 11.4 | 0.008 | Yes |
| | | SMARCA5 | FDDASP | | | | | |
| FUS12 | 23 | NUDT12-> | ARWFTREQ | 24.68 | 0.58 | 2.77 | 0.027 | Yes |
| | | LINC02115 | GSEN | | | | | |
| FUS13 | 25 | KCNQ5-> | AFIYHAFVG | 3.96 | 0.033 | 8.79 | 0.057 | Yes |
| | | KCNQ5-IT1 | STKTAWRTTL | | | | | |
| FUS14 | 27 | UBE2J1-> | LARQISFKSL | 3.27 | 0.005 | 256.93 | 0.19 | Yes |
| | | GABRR2 | I | | | | | |
| FUS16 | 31 | UBE2J1-> | PRDNHTDH | 8.86 | 0.12 | 4.388 | 0.027 | Yes |
| | | GABRR2 | ENPRGSDG | | | | | |
| | | | QGRWKCPS | | | | | |
| | | | QVTYIRRTL | | | | | |
| | | | M | | | | | |
| FUS17 | 33 | HBS1L-> | AAGVVTEIE | 5.31 | 0.13 | 2.11 | 0.012 | Yes |
| | | ALDH8A1 | AAVKA | | | | | |
| FUS18 | 35 | CADPS2-> | NAVRSYYEG | 542.51 | 0.671 | 0.95 | 0.005 | Yes |
| | | RNF148 | TENIVAV | | | | | |
| FUS19 | 37 | CADPS2-> | NAVRSYYETN | 12.96 | 0.016 | 1.26 | 0.009 | Yes |
| | | RNF148 | GGMSFLRITP | | | | | |
| FUS20 | 39 | PTGES2-> | ISKRLKSRRR | 511.064 | 4.85 | 11.664 | 0.068 | Yes |
| | | SLC25A25- | SGWQLNRA | | | | | |
| | | AS1 | GNRGLSPGL | | | | | |
| | | | GLFPRGCCR | | | | | |
| | | | WGGAYTRL | | | | | |
| | | | PSANQT | | | | | |
| FUS21 | 41 | FOSB-> | PPTAAASQT | 4.24 | 0.04 | 8.08 | 0.023 | Yes |
| | | KLF6 | CLELER | | | | | |
| FUS22 | 43 | IGLL5-> | ACGACGAG | 89.07 | 0.11 | 0.86 | 0.006 | Yes |
| | | COMMD3-BMI1, | FFIKQKCIEQ | | | | | |
| | | BMI1 | RESRSLS | | | | | |
| FUS23 | 45 | NDUFB8-> | VYPVYQPV | 42.81 | 0.064 | 1.94 | 0.011 | Yes |
| | | SEC31B | VSQAGLGEL | | | | | |
| | | | WQWASGK | | | | | |
| | | | LRGYCWRR | | | | | |
| | | | GQWHAYSI | | | | | |
| | | | QCDPHPVF | | | | | |
| | | | GEGACDCS | | | | | |
| | | | ETEAHGGC | | | | | |
| | | | QSPRLESFP | | | | | |
| | | | GQPPGFRG | | | | | |
| | | | QRF | | | | | |
| FU524 | 47 | B2M-> | SGLEAIQRG | 259.11 | 0.48 | 3.13 | 0.014 | Yes |
| | | DUSP5 | YETFYSE | | | | | |
| FUS26 | 51 | P2RY6-> | ARHLLTLGT | 7.07 | 0.1 | 2.46 | 0.026 | Yes |
| | | ARHGEF17 | CGSTWP | | | | | |
| FUS27 | 53 | TBCEL-> | PQEEVPFRS | 3.38 | 0.009 | 3.3 | 0.038 | Yes |
| | | TECTA | ITTELFPSMC | | | | | |
| FUS28 | 55 | NCOR2-> | QIIYDENRT | 247.49 | 0.37 | 1.13 | 0.009 | Yes |
| | | UBC | MQIFVKT | | | | | |
| FUS30 | 59 | ZFP36-> | MDLTAIYET | 3.5 | 0.13 | 51.81 | 0.1 | Yes |
| | | UBC | MQIFVKT | | | | | |
| FUS31 | 61 | FOSB-> | PPTAAASQT | 14.72 | 0.022 | 0.7 | 0.004 | Yes |
| | | UBC | MQIFVKT | | | | | |
| FUS32 | 63 | GANC-> | SSVTTHSSG | 12.04 | 0.018 | 3.08 | 0.008 | Yes |
| | | CAPN3 | NLRESPIYH | | | | | |
| FUS34 | 67 | CLN6-> | VAPSGLYYC | 8.7 | 0.013 | 8.33 | 0.015 | Yes |
| | | CALML4 | G | | | | | |
| FUS35 | 69 | TMED3-> | NRVTALTQ | 3.24 | 0.049 | 2.28 | 0.019 | Yes |
| | | KIAA1024 | N | | | | | |
| FUS36 | 71 | TNFRSF17-> | IEKSISARITE | 8.7 | 0.013 | 4.69 | 0.008 | Yes |
| | | SNX29 | Q | | | | | |
| FUS38 | 75 | CD79B-> | GEVKWSVG | 23.41 | 0.18 | 4.06 | 0.007 | Yes |
| | | GH1 | SRTSLLL | | | | | |
| FUS40 | 79 | KLF2-> | CRERGLQEA | 18.3 | 0.43 | 1.26 | 0.024 | Yes |
| | | TPM4 | EGDVAAL | | | | | |
| FUS41 | 81 | FOSB-> | PPTAAASQ | 37.46 | 0.056 | 6.84 | 0.002 | Yes |
| | | KLF2 | VRSPTTATG | | | | | |
| | | | TAAAGSLRA | | | | | |
| | | | QTSSRATTE | | | | | |
| | | | STRATGHSS | | | | | |
| | | | AICAIVPSRA | | | | | |
| | | | PITWRCT | | | | | |
| FUS42 | 83 | RBM42-> | GGPPFVGPL | 14.72 | 0.027 | 9.54 | 0.11 | Yes |
| | | ETV2 | RGPRAQPSL | | | | | |
| | | | SGLCGRRTG | | | | | |
| | | | SRDTIKIPG | | | | | |
| | | | QT | | | | | |
| FUS44 | 87 | ZGPAT-> | LDQCVETLC | 4.82 | 0.097 | 4.8 | 0.024 | Yes |
| | | LIME1 | SLSKSD | | | | | |
| FUS45 | 89 | IFNAR2-> | LLPPGQESA | 88.8 | 0.43 | 10.56 | 0.1 | Yes |
| | | IL10RB | IIGPPGMQ | | | | | |
| FUS46 | 91 | GCNA-> | YECTGCKTS | 143.71 | 1.33 | 9.3 | 0.017 | Yes |
| | | LOC101059915 | FPHRSQS | | | | | |
| FUS51 | 101 | POU2AF1-> | PPLITNVTEK | 200 | 0.06 | 11.93 | 0.061 | Yes |
| | | COLCA1 | PWKHTRIE | | | | | |
| | | | MPLARLTRP | | | | | |
| FUS52 | 103 | SELPLG-> | MEPTTKRGPG | 46.95 | 2.09 | 6.831 | 0.02 | Yes |
| | | TMEM119 | GTMVSAAAPS | | | | | |
| FUS53 | 105 | PTPRG-> | RPGVFTDIL | 76.5 | 0.78 | 6.29 | 0.041 | Yes |
| | | C3orf14 | | | | | | |
| FUS54 | 107 | ZFP36-> | MDLTAIYED | 15.176 | 2.58 | 9.66 | 0.063 | Yes |
| | | PLEKHG2 | SAGPLSHA | | | | | |
| FUS55 | 109 | FAM214A-> | LRYLIHLRK | 13.57 | 0.38 | 74.67 | 0.056 | Yes |
| | | ARPP19 | WKIK | | | | | |
| FUS56 | 111 | ZNF772-> | FALMASLGI | 2.73 | 0.007 | 115.03 | 0.75 | Yes |
| | | VN1R1 | PQTMAA | | | | | |
| FUS57 | 113 | MED12-> | RQLQQQLS | 17.2 | 0.14 | 11.65 | 0.24 | Yes |
| | | NLGN3 | SLSGPEGVP | | | | | |
| | | | FLKLWCLDD | | | | | |
| | | | LLSTLLGTCR | | | | | |
| | | | CPSRAQF | | | | | |
| AS3 | 119 | BBS9 | SLLSLFPGKT | 92.41 | 0.28 | 3.43 | 0.026 | Yes |
| | | | VEITCLQHQ | | | | | |
| | | | KC | | | | | |
| AS4 | 121 | BTBD3 | TQRLPGHK | 17.3 | 0.77 | 7.26 | 0.025 | Yes |
| | | | VSNSCMTG | | | | | |
| | | | LVLTFTKRD | | | | | |
| | | | PFHKPVTW | | | | | |
| | | | CGQLADVR | | | | | |
| | | | QCMFHSIRE | | | | | |
| | | | RAHPLQRAL | | | | | |
| | | | ATLNFFFVS | | | | | |
| | | | FYTALYLTH | | | | | |
| | | | PLLTLQTWE | | | | | |
| | | | VVVTVCFSC | | | | | |
| | | | WELCVPCD | | | | | |
| | | | VLRRTCRGQ | | | | | |
| | | | R | | | | | |
| AS5 | 123 | C3orf79 | LQAILQPGN | 4.64 | 0.068 | 19.14 | 0.14 | Yes |
| | | | FILVNEI | | | | | |
| AS6 | 125 | CTHRC1 | RQREVVDL | 2700 | 0.27 | 57.613 | 0.072 | Yes |
| | | | VSPREPSRD | | | | | |
| | | | RRAGGGDL | | | | | |
| | | | AARPTGRAS | | | | | |
| | | | VWLLGVSV | | | | | |
| | | | CTAVCRVSC | | | | | |
| | | | CAGVSCFLF | | | | | |
| | | | TGEEGPGYA | | | | | |
| | | | CSESWNSE | | | | | |
| | | | GRPKWEAI | | | | | |
| | | | VERTCDMG | | | | | |
| | | | PYNGMCLQ | | | | | |
| | | | G | | | | | |
| AS9 | 131 | GAS2 | GEKILFIRVK | 358.4 | 1.99 | 20.874 | 0.099 | Yes |
| | | | FYFSLILFLW | | | | | |
| | | | EDSEFELGEI | | | | | |
| AS10 | 133 | PDK1 | MKQFLDFG | 70.588 | 12 | 76.818 | 0.028 | Yes |
| | | | ECGPGPWA | | | | | |
| | | | FLRGPGRG | | | | | |
| | | | AAAAAPGR | | | | | |
| | | | VGAGQLSP | | | | | |
| | | | EAHPSSSAF | | | | | |
| | | | PPPAP | | | | | |
| AS11 | 135 | TSG101 | QLKKMVSK | 4 | 0.033 | 69.02 | 0.45 | Yes |
| | | | VRLRRARLP | | | | | |
| | | | GRAHRSLPR | | | | | |
| | | | PVESVPAQP | | | | | |
| | | | SKLPRRAGS | | | | | |
| | | | PGAVLSDLL | | | | | |
| | | | RTPPRGACR | | | | | |
| | | | LVRESPYGS | | | | | |
| | | | WEGGGRR | | | | | |
| | | | QSTKAWSG | | | | | |
| | | | GYLTPAAHP | | | | | |
| | | | PSSLPLSLEL | | | | | |
| | | | SVGQCSFSF | | | | | |
| | | | VFINTNKGS | | | | | |
| | | | YLVVDLRQS | | | | | |
| | | | STPVPI | | | | | |
| AS15 | 143 | B4GALT2 | YGVYVINQK | 290 | 0.029 | 2469.136 | 0.9 | Yes |
| | | | GHSGCIWS | | | | | |
| | | | MDGEGRLE | | | | | |
| | | | TAWPVWK | | | | | |
| | | | RGRCPGKR | | | | | |
| | | | R | | | | | |
| AS16 | 145 | CAMSAP1 | SRAPIKMSV | 8200 | 0.82 | 15.062 | 0.011 | Yes |
| | | | LGQGHSRCL | | | | | |
| | | | LHCLCLVAP | | | | | |
| | | | MAFPVRLR | | | | | |
| | | | VPCGG | | | | | |
| AS17 | 147 | DCC | SATTRSITG | 14.05 | 0.13 | 7.078 | 0.028 | Yes |
| | | | QDILSLEKER | | | | | |
| | | | R | | | | | |
| AS18 | 149 | FCRLB | LTALLLLAST | 7.33 | 0.082 | 12.922 | 0.006 | Yes |
| | | | PTP | | | | | |
| AS19 | 151 | GABRR2 | FSMRPAFG | 140 | 0.031 | 23.16 | 0.12 | Yes |
| | | | ETALGCKK | | | | | |
| | | | W | | | | | |
| AS23 | 157 | KMT2C | CKVCQNCK | 3.05 | 0.065 | 18 | 0.063 | Yes |
| | | | NPKMARAS | | | | | |
| AS24 | 161 | ST3GAL6 | DLFDEFDNS | 1300 | 0.13 | 613.88 | 3.14 | Yes |
| | | | RLVSRSWFF | | | | | |
| | | | FYLRNKWN | | | | | |
| | | | LALGF | | | | | |
| AS25 | 163 | TBCEL | RSISLHKSDP | 43.6 | 0.028 | 57.01 | 0.061 | Yes |
| | | | SLLSPN | | | | | |
| AS26 | 165 | AGFG1 | FLQKHGNE | 84.36 | 0.75 | 5.7 | 0.097 | Yes |
| | | | NTVLMLETL | | | | | |
| | | | SEIRYGYVSC | | | | | |
| | | | VCCPLLSPY | | | | | |
| | | | GRCLN | | | | | |
| AS29 | 171 | ARHGAP42 | SLLIGALRIT | 1100 | 0.29 | 15.34 | 0.1 | Yes |
| | | | WKREAGRK | | | | | |
| | | | SFFKCGLRV | | | | | |
| | | | HMWKGLQ | | | | | |
| | | | FIEITRRNW | | | | | |
| | | | MLIFQSCSL | | | | | |
| | | | FGTSQYRSL | | | | | |
| | | | TKKCQLCFR | | | | | |
| | | | FKRVSSKGS | | | | | |
| | | | DITDKREVT | | | | | |
| | | | H | | | | | |
| AS30 | 173 | BLNK | TVPASQKLS | 13.86 | 0.082 | 2.23 | 0.012 | Yes |
| | | | CSLDPKHLR | | | | | |
| | | | QFHL | | | | | |
| AS33 | 179 | CFAP54 | FGTSHMMV | 47.27 | 0.49 | 2.78 | 0.009 | Yes |
| | | | RHF | | | | | |
| AS36 | 185 | EPHA6 | EIEGSCHGG | 100 | 17 | 111.44 | 0.57 | Yes |
| | | | MITMDIKM | | | | | |
| | | | GSYSCDYLK | | | | | |
| | | | HSETLSAMV | | | | | |
| | | | VSCGISFLFL | | | | | |
| | | | FPRKKIAKLI | | | | | |
| | | | FKEQVP | | | | | |
| AS37 | 187 | AM13A | CEIMPLQSYI | 17.37 | 0.32 | 576.132 | 0.21 | Yes |
| | | | LL | | | | | |
| AS42 | 197 | LARP1B | VAPSQSRQ | 40 | 0.076 | 3.83 | 0.025 | Yes |
| | | | EECWRHKM | | | | | |
| | | | SW | | | | | |
| AS43 | 199 | LYSMD3 | IALQYCCTC | NA | NA | NA | NA | NA |
| | | | SISQ | | | | | |
| AS47 | 207 | NRG3 | TSTSPKFPVF | 53.5 | 0.25 | 1.907 | 0.011 | Yes |
| | | | IFRLVCWVR | | | | | |
| | | | TELVLR | | | | | |
| AS50 | 213 | PDZRN4 | KLLYEVSQK | 3.032 | 0.038 | 8.29 | 0.041 | Yes |
| | | | MVL | | | | | |
| AS52 | 217 | PRDM5 | SSLQEHRKL | 12.72 | 0.18 | 1.9 | 0.012 | Yes |
| | | | LS | | | | | |
| AS54 | 221 | SETBP1 | ERSHSKKKD | 0.73 | 0.008 | 90.535 | 0.033 | Yes |
| | | | GLVRNDGP | | | | | |
| | | | GFPPMFSTS | | | | | |
| | | | ENTNLD | | | | | |
| AS55 | 223 | TYW5 | AGDVLFIPGI | 96.05 | 0.17 | 27.71 | 0.042 | Yes |
| | | | SLKVHKVQ | | | | | |
| | | | HSKVINICN | | | | | |
| AS56 | 225 | ADGRE5 | IENEGGQVR | 5 | 0.069 | 3.71 | 0.019 | Yes |
| | | | SRPAPS | | | | | |
| AS64 | 241 | ITGA8 | DLNQDGYN | 6.83 | 0.8 | 376.11 | 0.57 | Yes |
| | | | GNLNQSVE | | | | | |
| | | | VIYVSSPLHK | | | | | |
| | | | GDHACFKV | | | | | |
| | | | FSMVCVGA | | | | | |
| | | | VIITDF | | | | | |
| AS67 | 247 | MTMR14 | PVILFKGKV | 28.23 | 0.52 | 8.57 | 0.071 | Yes |
| | | | RPIP | | | | | |
| AS68 | 249 | NFKB1 | QILEQPKQV | 4 | 0.022 | 21.77 | 0.033 | Yes |
| | | | RLKGWDFK | | | | | |
| | | | C | | | | | |
| AS73 | 259 | SMC5 | DMEVFLKE | 3 | 0.02 | 72.58 | 0.11 | Yes |
| | | | ASTNQHNT | | | | | |
| | | | LIHLTLTFST | | | | | |
| AS76 | 265 | TLE4 | QRDRDSIKV | 6.15 | 0.54 | 224.35 | 0.34 | Yes |
| | | | GLKIYRLRN | | | | | |
| | | | GLKGL | | | | | |
| AS77 | 267 | TRAF3 | KRYGCVFQ | 369.64 | 1.12 | 7.547 | 0.046 | Yes |
| | | | VSIRHLSFPV | | | | | |
| | | | TDILP | | | | | |
| AS78 | 269 | UBE2B | EGTPFEDGK | 29.32 | 0.52 | 3.14 | 0.024 | Yes |
| | | | SYSLCFL | | | | | |
| AS84 | 281 | ANKRD36 | SQKQPALKC | 162.14 | 4.54 | 10.66 | 0.019 | Yes |
| | | | LLRNNRP | | | | | |
| AS86 | 285 | ATP10B | VYYLYKNVA | 2.7 | 0.018 | 12.54 | 0.019 | Yes |
| | | | YKGSDID | | | | | |
| AS87 | 287 | BMP6 | QLSVVTRD | 44.64 | 1.25 | 4.45 | 0.016 | Yes |
| | | | DYNSSELK | | | | | |
| AS88 | 289 | CADPS2 | LEDKELGRIS | 4.55 | 0.025 | 79.18 | 0.12 | Yes |
| | | | VCPWTEGL | | | | | |
| | | | EKMEKTLLC | | | | | |
| | | | SSSG | | | | | |
| AS90 | 293 | CSAG1 | MSATTGSQ | 6327.68 | 11.2 | 79.18 | 0.12 | Yes |
| | | | DNPEGKRD | | | | | |
| | | | PSRKFQEQK | | | | | |
| | | | ALPKRPPLQ | | | | | |
| | | | KNLRNGVG | | | | | |
| | | | QHYPATLTS | | | | | |
| | | | RTRNSIKMR | | | | | |
| | | | HSRTTRARR | | | | | |
| | | | PPPSPVLPW | | | | | |
| | | | AASNSQAR | | | | | |
| | | | PETSSLSEKS | | | | | |
| | | | PEV | | | | | |
| AS92 | 297 | MSI2 | DPTTKRSRL | 41.24 | 0.073 | 31.67 | 0.048 | Yes |
| | | | TPKLHFLVE | | | | | |
| | | | RNPRWSQE | | | | | |
| | | | QRKYL | | | | | |
| AS93 | 299 | TJP1 | ILAQKKKDE | 1.44 | 0.014 | 9.9 | 0.015 | Yes |
| | | | LSS | | | | | |
| AS94 | 301 | TTLL7 | AKFWSDISI | 97.14 | 2.72 | 62.53 | 0.63 | Yes |
| | | | NRAPSFG | | | | | |
| AS95 | 303 | WDR64 | LDPPHDEKA | 1.58 | 0.13 | 19.58 | 0.014 | Yes |
| | | | LACPQWTLL | | | | | |
| | | | WIFWTAKA | | | | | |
| | | | L | | | | | |
| AS96 | 305 | ANKRD28 | KKEDVNFQ | 7.34 | 0.15 | 5.47 | 0.027 | Yes |
| | | | VKQLIH | | | | | |
| AS97 | 307 | ANKRD28 | GNVLVRYVS | 13446.33 | 23.8 | 96.12 | 0.57 | Yes |
| | | | LYVYYILLN | | | | | |
| AS108 | 329 | ACIN1 | VEDEEKKEA | 1406.07 | 12.5 | 179.31 | 1.06 | Yes |
| | | | GTHFIHLTG | | | | | |
| | | | TTVSAGVPE | | | | | |
| | | | EMPATTLRR | | | | | |
| | | | EVF | | | | | |
| AS109 | 331 | ADAMTS15 | TAILFTRQQ | 4.15 | 0.019 | 42.23 | 0.064 | Yes |
| | | | SKALPSLGIT | | | | | |
| | | | NRHGFWA | | | | | |
| | | | GQGNTVM | | | | | |
| | | | HGRDSDGK | | | | | |
| | | | QVKGTVVIL | | | | | |
| | | | AQGAFHQ | | | | | |
| AS110 | 333 | CCDC88A | NASLHEVKD | 228.56 | 4.21 | 36.021 | 0.21 | Yes |
| | | | FFVPFPLPLL | | | | | |
| | | | HLTSLKRNK | | | | | |
| | | | LFN | | | | | |
| AS111 | 335 | CD55 | GTTRLLSGE | 1.16 | 0.012 | 16.71 | 0.06 | Yes |
| | | | DFS | | | | | |
| AS112 | 337 | CFAP54 | SEMVAHER | 35.03 | 0.062 | 7.92 | 0.012 | Yes |
| | | | YVFEATGN | | | | | |
| | | | DSFRTLCLL | | | | | |
| | | | MK | | | | | |
| AS113 | 339 | CLCC1 | IMALAILDLF | 5.07 | 0.38 | 5.7 | 0.009 | Yes |
| | | | AVLKIEAHK | | | | | |
| | | | QLLLKSFCY | | | | | |
| | | | GAGK | | | | | |
| AS114 | 341 | CNTN5 | QIRVPSYSD | 7.6 | 0.14 | 2.05 | 0.02 | Yes |
| | | | EG | | | | | |
| AS115 | 343 | CTHRC1 | RQREVVDL | 288.14 | 0.51 | 223.78 | 0.16 | Yes |
| | | | VKKDLVMR | | | | | |
| | | | AVSLGTQR | | | | | |
| | | | GDQNGRPS | | | | | |
| | | | WSGHAIWA | | | | | |
| | | | RIMECAYKG | | | | | |
| | | | QQECLVET | | | | | |
| | | | GALGPMAF | | | | | |
| | | | RVHLGSQV | | | | | |
| | | | GMDSKEKR | | | | | |
| | | | GNV | | | | | |
| AS116 | 345 | FCRL5 | VHLDFSSAE | 192.71 | 1.42 | 6.32 | 0.076 | Yes |
| | | | MGFPHAAQ | | | | | |
| | | | ANVELLGSS | | | | | |
| | | | DLLT | | | | | |
| AS118 | 349 | IARS | GFFETEMA | 106.428 | 1.01 | 7.692 | 0.15 | Yes |
| | | | VPFFSRKIRK | | | | | |
| | | | VRRAIMKW | | | | | |
| | | | TFCCYFY | | | | | |
| AS120 | 353 | NCAM1 | WTRPEKQE | 13.03 | 0.24 | 3.71 | 0.018 | Yes |
| | | | LNSSCCSLIL | | | | | |
| AS121 | 355 | OGT | LGRLEEAKS | 194.72 | 0.59 | 1.41 | 0.02 | Yes |
| | | | FETEEN | | | | | |
| AS122 | 357 | OGT | LGRLEEAKV | 3.63 | 0.044 | 13.71 | 0.048 | Yes |
| | | | PLKMLLF | | | | | |
| AS123 | 359 | PCNX3 | WLLRTWER | 5.371 | 0.046 | 2.25 | 0.05 | No |
| | | | ADSGL | | | | | |
| AS124 | 361 | PDZRN4 | LVARPEIQIF | 5.27 | 0.089 | 7.31 | 0.064 | Yes |
| | | | MEEDVMK | | | | | |
| | | | VE | | | | | |
| AS125 | 363 | PRDM5 | RHQENVHT | 28.62 | 0.78 | 6.57 | 0.023 | Yes |
| | | | VLVHGKVK | | | | | |
| | | | GLFYI | | | | | |
| AS126 | 365 | RAB2B | IMLIGNKSN | 161.72 | 0.49 | 6.861 | 0.04 | Yes |
| | | | CQKQLLRD | | | | | |
| | | | MKILNEFYH | | | | | |
| | | | GLILTLQLS | | | | | |
| AS127 | 367 | ST3GAL6 | PKVFPKNQ | 1.44 | 0.08 | 7.179 | 0.14 | Yes |
| | | | VISLIPKAQE | | | | | |
| | | | DPEHT | | | | | |
| AS128 | 369 | STARD5 | GWKICREGI | 874.02 | 7.77 | 44.35 | 0.081 | Yes |
| | | | GPVMLTMT | | | | | |
| | | | SGEWSFSFL | | | | | |
| | | | EAICGVSRE | | | | | |
| | | | PVPRRRHCI | | | | | |
| | | | WDTRGGV | | | | | |
| | | | GLCEASCW | | | | | |
| | | | RPTSEVG | | | | | |
| AS129 | 371 | TET1 | IDPSSPLHTY | 19.08 | 0.27 | 9.79 | 0.022 | Yes |
| | | | YERITKGRN | | | | | |
| | | | PERRYMKP | | | | | |
| | | | ERISPGHEA | | | | | |
| | | | MEKNLEDN | | | | | |
| | | | L | | | | | |
| AS131 | 375 | USP25 | QILQQALKK | 299.44 | 0.53 | 10.53 | 0.008 | Yes |
| | | | LPFSLCITEC | | | | | |
| | | | ETIAYHLAR | | | | | |
| | | | DSNGNLEL | | | | | |
| AS132 | 377 | USP25 | QILQQALKI | 4.73 | 0.026 | 181.82 | 0.17 | Yes |
| | | | AFLKVVVPL | | | | | |
| | | | KLHPYSFCH | | | | | |
| AS133 | 379 | WNT10A | YESPIFSRDR | 38.74 | 0.095 | 12.303 | 0.071 | Yes |
| | | | VNPGALSGS | | | | | |
| | | | LGQARAAC | | | | | |
| | | | WRTLGSLKP | | | | | |
| | | | GGEFRAAE | | | | | |
| | | | QRRAGWVP | | | | | |
| | | | GAPGFTCSL | | | | | |
| | | | ISSRFPRERF | | | | | |
| | | | CLRHRSSW | | | | | |
| | | | RGARRVQC | | | | | |
| | | | VCPGQTEG | | | | | |
| | | | LWL | | | | | |
| AS134 | 381 | XPO1 | FSQNMNTK | 4.51 | 0.091 | 1.429 | 0.02 | Yes |
| | | | MIRGTERK | | | | | |
| | | | QQYYGLQIL | | | | | |
| | | | E | | | | | |
| AS135 | 383 | ZBP1 | GEPGEDAGI | 2.68 | 0.092 | 3.73 | 0.03 | No |
| | | | GRPEERERE | | | | | |
| | | | EEMADQ | | | | | |
| AS136 | 385 | ZNF592 | PPGGHSPQ | 5.91 | 0.072 | 10.86 | 0.07 | Yes |
| | | | DLSIREIFCL | | | | | |
| | | | GHKEVC | | | | | |
| AS138 | 421 | BTBD3 | TQRLPGHK | NA | NA | NA | NA | NA |
| | | | VSNSCMTG | | | | | |
| | | | LVLTFTKRD | | | | | |
| | | | PFHKPVTW | | | | | |
| | | | CGQLADVR | | | | | |
| | | | QCMFHSIRE | | | | | |
| | | | RAHPLQRAL | | | | | |
| | | | ATLNFFFVS | | | | | |
| | | | FYTALYLTH | | | | | |
| | | | PLLTLQTWE | | | | | |
| | | | VVVTGQES | | | | | |
| | | | RMFTLLLEIS | | | | | |
| | | | YVSYYCMYL | | | | | |
| | | | VMRGHVH | | | | | |

### Example 4: HLA Binding Predictions

Amino acid sequences of neoantigens identified using the various approaches as described in Example 1 are split into all possible unique, contiguous 9 mer amino acid fragments and HLA binding predictions to six common HLA alleles (HLA-A*01:01, HLA-A*02:01, HLA-A*03:01, HLA-A*24:02, HLA-B*07:02, HLA-B*08:01) are performed for each of these 9 mers using netMHCpan4.0. Several 9 mer fragments are selected for further analysis based on ranking by likelihood of binding to one or more of the tested HLA alleles and their prevalence in multiple myeloma patients.

### Example 5: In Vitro Binding of Neoantigens to HLA

Binding of select neoantigens of fragments thereof to HLA-A*01:01, HLA-A*02:01, HLA-A*03:01, HLA-A*24:02, HLA-B*07:02 and HLA-B*08:01 or any other HLA is evaluated using known methods. The principle of the method is briefly described below and consists of two parts, one involving exchange of peptide with a positive control induced by Ultraviolet (UV) radiation, and the second is an enzyme immunoassay to detect stable HLA-peptide and empty HLA complexes.

HLA-bound peptides are critical for the stability of the HLA complex. A conditional HLA class I complex is stabilized by an UV-labile peptide utilizing a different peptide (Pos) for each HLA (Pos: HLA-A*01:01: CTELKLSDY(SEQ ID NO: 409), HLA-A*02:01: NLVPMVATV (SEQ ID NO: 410), HLA-A*03:01: LIYRRRLMK (SEQ ID NO: 411), HLA-A*24:02: LYSACFWWL (SEQ ID NO: 412), HLA-B*07:02: NPKASLLSL (SEQ ID NO: 413), HLA-B*08:01: ELRSRYWAI (SEQ ID NO: 414)), which could be cleaved by UV irradiation when bound to the HLA molecule. Upon cleavage, the resulting peptide fragments dissociate from the HLA class I complex since their length is insufficient to bind stably to HLA. Under the conditions in which peptide cleavage is performed (neutral pH, on melting ice), the peptide-free HLA complex remains stable. Thus, when cleavage is performed in the presence of another HLA class I peptide of choice, this reaction results in net exchange of the cleaved UV-labile peptide Pos with the chosen peptide (Rodenko, B et al. (2006) Nature Protocols 1: 1120-32, Toebes, M et al. (2006) Nat Med 12: 246-51, Bakker, AH et al. (2008) Proc Natl Acad Sci USA 105: 3825-30).

The exchange efficiency between the peptide of interest and Pos is analyzed using an HLA class I ELISA. The combined technologies allow the identification of ligands for an HLA molecule of interest which are potentially immunogenic.

Exchange control peptide Pos is a high affinity binder to the relevant HLA class I allele while exchange control peptide Neg is a non-binder. UV control represents UV-irradiation of conditional HLA class I complex in the absence of a rescue peptide. Binding of exchange control peptide Neg (HLA-A*01:01: NPKASLLSL (SEQ ID NO: 415), HLA-A*02-01: IVTDFSVIK (SEQ ID NO: 416), HLA-A*03:01: NPKASLLSL (SEQ ID NO: 417), HLA-A*24:02: NLVPMVATV (SEQ ID NO: 418), HLA-B*07:02: LIYRRRLMK (SEQ ID NO: 419), HLA-B*08:01: NLVPMVATV (SEQ ID NO: 420)) and all experimental peptides are evaluated relative to that of exchange control peptide Pos. The absorption of the latter peptide is set at 100%. This procedure results in a range of different exchange percentages that reflects the affinities of the different experimental peptides for the HLA allele used.

HLA class I ELISA is an enzyme immunoassay based on the detection of beta2-microglobulin (B2M) of (peptide-stabilized) HLA class I complexes. To this end streptavidin is bound onto polystyrene microtiter wells. After washing and blocking, HLA complex present in exchange reaction mixtures or ELISA controls is captured by the streptavidin on the microtiter plate via its biotinylated heavy chain. Non-bound material is removed by washing. Subsequently, horseradish peroxidase (HRP)-conjugated antibody to human B2M is added. The HRP-conjugated antibody binds only to an intact HLA complex present in the microtiter well because unsuccessful peptide exchange results in disintegration of the original UV-sensitive HLA complex upon UV illumination. In the latter case B2M is removed during the washing step. After removal of non-bound HRP conjugate by washing, a substrate solution is added to the wells. A colored product forms in proportion to the amount of intact HLA complex present in the samples. After the reaction is terminated by the addition of a stop solution, absorbance is measured in a microtiter plate reader. The absorbance is normalized to the absorbance of an exchange control peptide (represents 100%). Suboptimal HLA binding of peptides with a moderate to low affinity for HLA class I molecules can also be detected by this ELISA technique (Rodenko, B et al. (2006) Nature Protocols 1: 1120-32).

According to the protocol described herein, HLA allele that is tested has a corresponding positive control (Pos) and a negative control (Neg) peptide against which the peptide of interest is exchanged. An exchange rate of 100% with Pos means that the peptide of interest has the same binding affinity to the HLA allele as the positive control peptide. Peptides with an exchange rate of at least 10% with the corresponding Pos peptide for at least one of the 6 HLA alleles are considered for further evaluation. Higher percentages correspond to stronger binding to the HLA allele.

### Embodiments

The following list of embodiments is intended to complement, rather than displace or supersede, the previous descriptions.

Embodiment 1. A polypeptide comprising at least one or more peptides sequences selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, or 405, 407, or 421 or fragments thereof.

Embodiment 2. A polypeptide comprising at least one or more peptides sequences selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

Embodiment 3. A polypeptide comprising two or more tandem repeats of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

Embodiment 4. The polypeptide of any of one of embodiments 1-3, wherein the polypeptide sequences are connected to each other in any order.

Embodiment 5. The polypeptide of embodiment 2, wherein the polypeptide is selected from:

an amino acid sequence of SEQ ID NO: 7 or having at least 90% sequence identity to SEQ ID NO: 7;

an amino acid sequence of SEQ ID NO: 9 or having at least 90% sequence identity to SEQ ID NO: 9;

an amino acid sequence of SEQ ID NO: 11 or having at least 90% sequence identity to SEQ ID NO: 11;

an amino acid sequence of SEQ ID NO: 13 or having at least 90% sequence identity to SEQ ID NO: 13;

an amino acid sequence of SEQ ID NO: 17 or having at least 90% sequence identity to SEQ ID NO: 17;

an amino acid sequence of SEQ ID NO: 21 or having at least 90% sequence identity to SEQ ID NO: 21;

an amino acid sequence of SEQ ID NO: 23 or having at least 90% sequence identity to SEQ ID NO: 23;

an amino acid sequence of SEQ ID NO: 25 or having at least 90% sequence identity to SEQ ID NO: 25;

an amino acid sequence of SEQ ID NO: 27 or having at least 90% sequence identity to SEQ ID NO: 27;

an amino acid sequence of SEQ ID NO: 31 or having at least 90% sequence identity to SEQ ID NO: 31;

an amino acid sequence of SEQ ID NO: 33 or having at least 90% sequence identity to SEQ ID NO: 33;

an amino acid sequence of SEQ ID NO: 35 or having at least 90% sequence identity to SEQ ID NO: 35;

an amino acid sequence of SEQ ID NO: 37 or having at least 90% sequence identity to SEQ ID NO: 37;

an amino acid sequence of SEQ ID NO: 39 or having at least 90% sequence identity to SEQ ID NO: 39;

an amino acid sequence of SEQ ID NO: 41 or having at least 90% sequence identity to SEQ ID NO: 41;

an amino acid sequence of SEQ ID NO: 43 or having at least 90% sequence identity to SEQ ID NO: 43;

an amino acid sequence of SEQ ID NO: 45 or having at least 90% sequence identity to SEQ ID NO: 45;

an amino acid sequence of SEQ ID NO: 47 or having at least 90% sequence identity to SEQ ID NO: 47;

an amino acid sequence of SEQ ID NO: 51 or having at least 90% sequence identity to SEQ ID NO: 51;

an amino acid sequence of SEQ ID NO: 53 or having at least 90% sequence identity to SEQ ID NO: 53;

an amino acid sequence of SEQ ID NO: 55 or having at least 90% sequence identity to SEQ ID NO: 55;

an amino acid sequence of SEQ ID NO: 59 or having at least 90% sequence identity to SEQ ID NO: 59;

an amino acid sequence of SEQ ID NO: 61 or having at least 90% sequence identity to SEQ ID NO: 61;

an amino acid sequence of SEQ ID NO: 63 or having at least 90% sequence identity to SEQ ID NO: 63;

an amino acid sequence of SEQ ID NO: 67 or having at least 90% sequence identity to SEQ ID NO: 67;

an amino acid sequence of SEQ ID NO: 69 or having at least 90% sequence identity to SEQ ID NO: 69;

an amino acid sequence of SEQ ID NO: 71 or having at least 90% sequence identity to SEQ ID NO: 71;

an amino acid sequence of SEQ ID NO: 75 or having at least 90% sequence identity to SEQ ID NO: 75;

an amino acid sequence of SEQ ID NO: 79 or having at least 90% sequence identity to SEQ ID NO: 79;

an amino acid sequence of SEQ ID NO: 81 or having at least 90% sequence identity to SEQ ID NO: 81;

an amino acid sequence of SEQ ID NO: 83 or having at least 90% sequence identity to SEQ ID NO: 83;

an amino acid sequence of SEQ ID NO: 87 or having at least 90% sequence identity to SEQ ID NO: 87;

an amino acid sequence of SEQ ID NO: 89 or having at least 90% sequence identity to SEQ ID NO: 89;

an amino acid sequence of SEQ ID NO: 91 or having at least 90% sequence identity to SEQ ID NO: 91;

an amino acid sequence of SEQ ID NO: 101 or having at least 90% sequence identity to SEQ ID NO: 101;

an amino acid sequence of SEQ ID NO: 103 or having at least 90% sequence identity to SEQ ID NO: 103;

an amino acid sequence of SEQ ID NO: 105 or having at least 90% sequence identity to SEQ ID NO: 105;

an amino acid sequence of SEQ ID NO: 109 or having at least 90% sequence identity to SEQ ID NO: 109;

an amino acid sequence of SEQ ID NO: 111 or having at least 90% sequence identity to SEQ ID NO: 111;

an amino acid sequence of SEQ ID NO: 113 or having at least 90% sequence identity to SEQ ID NO: 113;

an amino acid sequence of SEQ ID NO: 119 or having at least 90% sequence identity to SEQ ID NO: 119;

an amino acid sequence of SEQ ID NO: 121 or having at least 90% sequence identity to SEQ ID NO: 121;

an amino acid sequence of SEQ ID NO: 123 or having at least 90% sequence identity to SEQ ID NO: 123;

an amino acid sequence of SEQ ID NO: 125 or having at least 90% sequence identity to SEQ ID NO: 125;

an amino acid sequence of SEQ ID NO: 131 or having at least 90% sequence identity to SEQ ID NO: 131;

an amino acid sequence of SEQ ID NO: 133 or having at least 90% sequence identity to SEQ ID NO: 133;

an amino acid sequence of SEQ ID NO: 135 or having at least 90% sequence identity to SEQ ID NO: 135;

an amino acid sequence of SEQ ID NO: 143 or having at least 90% sequence identity to SEQ ID NO: 143;

an amino acid sequence of SEQ ID NO: 145 or having at least 90% sequence identity to SEQ ID NO: 145;

an amino acid sequence of SEQ ID NO: 147 or having at least 90% sequence identity to SEQ ID NO: 147;

an amino acid sequence of SEQ ID NO: 149 or having at least 90% sequence identity to SEQ ID NO: 149;

an amino acid sequence of SEQ ID NO: 151 or having at least 90% sequence identity to SEQ ID NO: 151;

an amino acid sequence of SEQ ID NO: 157 or having at least 90% sequence identity to SEQ ID NO: 157;

an amino acid sequence of SEQ ID NO: 161 or having at least 90% sequence identity to SEQ ID NO: 161;

an amino acid sequence of SEQ ID NO: 163 or having at least 90% sequence identity to SEQ ID NO: 163;

an amino acid sequence of SEQ ID NO: 165 or having at least 90% sequence identity to SEQ ID NO: 165;

an amino acid sequence of SEQ ID NO: 171 or having at least 90% sequence identity to SEQ ID NO: 171;

an amino acid sequence of SEQ ID NO: 173 or having at least 90% sequence identity to SEQ ID NO: 173;

an amino acid sequence of SEQ ID NO: 179 or having at least 90% sequence identity to SEQ ID NO: 179;

an amino acid sequence of SEQ ID NO: 185 or having at least 90% sequence identity to SEQ ID NO: 185;

an amino acid sequence of SEQ ID NO: 187 or having at least 90% sequence identity to SEQ ID NO: 187;

an amino acid sequence of SEQ ID NO: 197 or having at least 90% sequence identity to SEQ ID NO: 197;

an amino acid sequence of SEQ ID NO: 199 or having at least 90% sequence identity to SEQ ID NO: 199;

an amino acid sequence of SEQ ID NO: 207 or having at least 90% sequence identity to SEQ ID NO: 207;

an amino acid sequence of SEQ ID NO: 213 or having at least 90% sequence identity to SEQ ID NO: 213;

an amino acid sequence of SEQ ID NO: 217 or having at least 90% sequence identity to SEQ ID NO: 217;

an amino acid sequence of SEQ ID NO: 221 or having at least 90% sequence identity to SEQ ID NO: 221;

an amino acid sequence of SEQ ID NO: 223 or having at least 90% sequence identity to SEQ ID NO: 223;

an amino acid sequence of SEQ ID NO: 225 or having at least 90% sequence identity to SEQ ID NO: 225;

an amino acid sequence of SEQ ID NO: 241 or having at least 90% sequence identity to SEQ ID NO: 241;

an amino acid sequence of SEQ ID NO: 247 or having at least 90% sequence identity to SEQ ID NO: 247;

an amino acid sequence of SEQ ID NO: 249 or having at least 90% sequence identity to SEQ ID NO: 249;

an amino acid sequence of SEQ ID NO: 259 or having at least 90% sequence identity to SEQ ID NO: 259;

an amino acid sequence of SEQ ID NO: 265 or having at least 90% sequence identity to SEQ ID NO: 265;

an amino acid sequence of SEQ ID NO: 267 or having at least 90% sequence identity to SEQ ID NO: 267;

an amino acid sequence of SEQ ID NO: 269 or having at least 90% sequence identity to SEQ ID NO: 269;

an amino acid sequence of SEQ ID NO: 281 or having at least 90% sequence identity to SEQ ID NO: 281;

an amino acid sequence of SEQ ID NO: 285 or having at least 90% sequence identity to SEQ ID NO: 285;

an amino acid sequence of SEQ ID NO: 287 or having at least 90% sequence identity to SEQ ID NO: 287;

an amino acid sequence of SEQ ID NO: 289 or having at least 90% sequence identity to SEQ ID NO: 289;

an amino acid sequence of SEQ ID NO: 293 or having at least 90% sequence identity to SEQ ID NO: 293;

an amino acid sequence of SEQ ID NO: 297 or having at least 90% sequence identity to SEQ ID NO: 297;

an amino acid sequence of SEQ ID NO: 299 or having at least 90% sequence identity to SEQ ID NO: 299;

an amino acid sequence of SEQ ID NO: 301 or having at least 90% sequence identity to SEQ ID NO: 301;

an amino acid sequence of SEQ ID NO: 303 or having at least 90% sequence identity to SEQ ID NO: 303;

an amino acid sequence of SEQ ID NO: 305 or having at least 90% sequence identity to SEQ ID NO: 305;

an amino acid sequence of SEQ ID NO: 307 or having at least 90% sequence identity to SEQ ID NO: 307;

an amino acid sequence of SEQ ID NO: 329 or having at least 90% sequence identity to SEQ ID NO: 329;

an amino acid sequence of SEQ ID NO: 331 or having at least 90% sequence identity to SEQ ID NO: 331;

an amino acid sequence of SEQ ID NO: 333 or having at least 90% sequence identity to SEQ ID NO: 333;

an amino acid sequence of SEQ ID NO: 335 or having at least 90% sequence identity to SEQ ID NO: 335;

an amino acid sequence of SEQ ID NO: 337 or having at least 90% sequence identity to SEQ ID NO: 337;

an amino acid sequence of SEQ ID NO: 339 or having at least 90% sequence identity to SEQ ID NO: 339;

an amino acid sequence of SEQ ID NO: 341 or having at least 90% sequence identity to SEQ ID NO: 341;

an amino acid sequence of SEQ ID NO: 343 or having at least 90% sequence identity to SEQ ID NO: 343;

an amino acid sequence of SEQ ID NO: 345 or having at least 90% sequence identity to SEQ ID NO: 345;

an amino acid sequence of SEQ ID NO: 349 or having at least 90% sequence identity to SEQ ID NO: 349;

an amino acid sequence of SEQ ID NO: 359 or having at least 90% sequence identity to SEQ ID NO: 359;

an amino acid sequence of SEQ ID NO: 361 or having at least 90% sequence identity to SEQ ID NO: 361;

an amino acid sequence of SEQ ID NO: 363 or having at least 90% sequence identity to SEQ ID NO: 363;

an amino acid sequence of SEQ ID NO: 365 or having at least 90% sequence identity to SEQ ID NO: 365;

an amino acid sequence of SEQ ID NO: 367 or having at least 90% sequence identity to SEQ ID NO: 367;

an amino acid sequence of SEQ ID NO: 369 or having at least 90% sequence identity to SEQ ID NO: 369;

an amino acid sequence of SEQ ID NO: 371 or having at least 90% sequence identity to SEQ ID NO: 371;

an amino acid sequence of SEQ ID NO: 375 or having at least 90% sequence identity to SEQ ID NO: 375;

an amino acid sequence of SEQ ID NO: 377 or having at least 90% sequence identity to SEQ ID NO: 377;

an amino acid sequence of SEQ ID NO: 379 or having at least 90% sequence identity to SEQ ID NO: 379;

an amino acid sequence of SEQ ID NO: 381 or having at least 90% sequence identity to SEQ ID NO: 381;

an amino acid sequence of SEQ ID NO: 383 or having at least 90% sequence identity to SEQ ID NO: 383;

an amino acid sequence of SEQ ID NO: 385 or having at least 90% sequence identity to SEQ ID NO: 385;

an amino acid sequence of SEQ ID NO: 385 or having at least 90% sequence identity to SEQ ID NO: 385;

an amino acid sequence of SEQ ID NO: 421 or having at least 90% sequence identity to SEQ ID NO: 421;

and combinations thereof.

Embodiment 6. A polynucleotide encoding a polypeptide of any one of the embodiments 1-5.

Embodiment 7. The polynucleotide of embodiment 6, wherein the polynucleotide is selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, or 406, 408, or 422, or fragments thereof.

Embodiments 8. A vector comprising a polynucleotide of embodiment 6 or embodiment 7.

Embodiments 9. The vector of embodiment 8, wherein the vector is selected from an adenovirus vector, an alphaviral vector, a poxvirus vector, an adeno-associated virus vector, a retrovirus vector, a self-replicating RNA molecule, and a combination thereof.

Embodiment 10. The vector of embodiment 9, wherein the adenovirus vector is selected from hAd5, hAd7, hAd11, hAd26, hAd34, hAd35, hAd48, hAd49, hAd50, GAd20, Gad19, GAd21, GAd25, GAd26, GAd27, GAd28, GAd29, GAd30, GAd31, ChAd3, ChAd4, ChAd5, ChAd6, ChAd7, ChAd8, ChAd9, ChAd10, ChAd11, ChAd16, ChAdI7, ChAd19, ChAd20, ChAd22, ChAd24, ChAd26, ChAd30, ChAd31, ChAd37, ChAd38, ChAd44, ChAd55, ChAd63, ChAd73, ChAd82, ChAd83, ChAd146, ChAd147, PanAdl, PanAd2, and PanAd3.

Embodiment 11. The vector of embodiment 9, wherein the poxvirus vector is selected from smallpox virus vector, vaccinia virus vector, cowpox virus vector, monkeypox virus vector, Copenhagen vaccinia virus (W) vector, New York Attenuated Vaccinia Virus (NYVAC) vector, and Modified Vaccinia Ankara (MVA) vector.

Embodiment 12. The vector of embodiment 9, wherein the vector is the adenovirus vector comprising a polynucleotide encoding any one of the polypeptides of any one of embodiments 1-5.

Embodiment 13. The vector of embodiment 9, wherein the vector is the poxvirus vector comprising a polynucleotide encoding any one of the polypeptides of any one of embodiments 1-5.

Embodiment 14. The vector of embodiment 9, wherein the vector is the self-replicating RNA molecule comprising a polynucleotide encoding any one of the polypeptides of any one of embodiments 1-5.

Embodiment 15. A pharmaceutical composition comprising a polypeptide of any one of embodiments 1-5.

Embodiment 16. A pharmaceutical composition comprising a polynucleotide of any one of embodiments 6 and 7.

Embodiment 17. A pharmaceutical composition comprising a vector of any one of embodiments 8-14.

Embodiment 18. The pharmaceutical composition of embodiment 17, wherein the vector is selected from an Ad26 vector, a MVA vector, a GAd20 vector, a self-replicating RNA molecule, and combinations thereof.

Embodiment 19. The pharmaceutical composition of embodiment 18, wherein the vector is an Ad26 vector comprising

a polynucleotide encoding one or more polypeptides selected from the group consisting of SEQ ID NOs 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, or

a polynucleotide encoding one or more polypeptides having at least 90% sequence identity to SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

Embodiment 20. The pharmaceutical composition of embodiment 18, wherein the vector is an GAd20 vector comprising

a polynucleotide encoding one or more polypeptides selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, or

a polynucleotide encoding one or more polypeptides having at least 90% sequence identity to SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

Embodiment 21. The pharmaceutical composition of embodiment 18, wherein the vector is an MVA vector comprising

a polynucleotide encoding one or more polypeptides selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, or

a polynucleotide encoding one or more polypeptides having at least 90% sequence identity to SEQ ID NOs:7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

Embodiment 22. The pharmaceutical composition of embodiment 18, wherein the vector is a self-replicating RNA molecule comprising

a polynucleotide encoding one or more polypeptides selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, or

a polynucleotide encoding one or more polypeptides having at least 90% sequence identity to SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

Embodiment 23. A method of inducing an immune response in a subject comprising administering to the subject in need thereof a pharmaceutical composition of any one of embodiments 15-22.

Embodiment 24. A method of inducing an immune response in a subject comprising administering to the subject in need thereof a composition comprising a recombinant virus and/or a composition comprising a self-replicating RNA molecule, wherein the recombinant virus or the self-replicating RNA molecule comprises a polynucleotide encoding at least one or more polypeptide selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

Embodiment 25. The method of embodiment 23 or 24, wherein the subject expresses or is suspected to express one or more polypeptides of claim 1.

Embodiment 26. A method of treating, preventing, reducing a risk of onset or delaying the onset of multiple myeloma in a subject comprising administering to the subject in need thereof a pharmaceutical composition of any one of embodiments 15-22.

Embodiment 27. A method of treating, preventing, reducing a risk of onset or delaying the onset of multiple myeloma in a subject comprising administering to the subject in need thereof a composition comprising a recombinant virus and/or a composition comprising a self-replicating RNA molecule, wherein the recombinant virus or the self-replicating RNA molecule comprises a polynucleotide encoding at least one or more polypeptides selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

Embodiment 28. A method of treating, preventing, reducing a risk of onset or delaying the onset of multiple myeloma in a subject comprising administering to the subject in need thereof a composition comprising a recombinant virus and/or a composition comprising a self-replicating RNA molecule, wherein the recombinant virus or the self-replicating RNA molecule comprises a polynucleotide encoding at least one or more polypeptides selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, and wherein the administration comprises one or more administrations of the composition.

Embodiment 29. The method of any one of embodiments 23-28. wherein the virus or recombinant virus is selected from Ad26, MVA, GAd20, and combinations thereof.

Embodiment 30. The method of embodiment 29, wherein the recombinant virus is an Ad26 virus comprising a polynucleotide encoding one or more polypeptides from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

Embodiment 31. The method of embodiment 29, wherein the recombinant virus is a GAd20 virus comprising a polynucleotide encoding one or more polypeptides from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

Embodiment 32. The method of embodiment 29, wherein the recombinant virus is a MVA virus comprising a polynucleotide encoding one or more polypeptides from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

Embodiment 33. The method of embodiment 29, wherein the self-replicating RNA molecule comprising a polynucleotide encoding one or more polypeptides from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

Embodiment 34. The method of any one of embodiments 23-33, comprising one or more treatment cycles, wherein each cycle comprises:

a first administration comprising a first composition comprising a recombinant virus or a self-replicating RNA molecule comprising a polynucleotide encoding one or more polypeptides from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, and wherein the recombinant virus is selected from Ad26, MVA, or GAd20; and

a second administration comprising a second composition comprising a recombinant virus or a self-replicating RNA molecule comprising a polynucleotide encoding one or more polypeptides from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, and wherein the recombinant virus is selected from Ad26, MVA, or GAd20.

Embodiment 35. The method of any one of embodiments 23-33, comprising one or more treatment cycles, wherein each cycle comprises:

a first administration comprising a first composition comprising or recombinant virus or a self-replicating RNA molecule comprising a polynucleotide encoding one or more polypeptides from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, and wherein the recombinant virus is selected from Ad26, MVA, or GAd20; and

a second administration comprising a second composition comprising a recombinant virus or a self-replicating RNA molecule comprising a polynucleotide encoding one or more polypeptides from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, and wherein the recombinant virus is selected from Ad26, MVA, or GAd20; and

a third administration comprising a second composition comprising a recombinant virus comprising a polynucleotide encoding one or more polypeptides from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, and wherein the recombinant virus is selected from Ad26, MVA, or GAd20.

Embodiment 36. The method of any one of embodiments 23-33, comprising one or more treatment cycles, wherein each cycle comprises:

a first administration comprising a first composition comprising a recombinant virus or a self-replicating RNA molecule comprising a polynucleotide encoding one or more polypeptides from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, and wherein the recombinant virus is selected from Ad26, MVA, or GAd20; and

a second administration comprising a second composition comprising a recombinant virus or a self-replicating RNA molecule comprising a polynucleotide encoding one or more polypeptides from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, and wherein the recombinant virus is selected from Ad26, MVA, or GAd20; and

a third administration comprising a second composition comprising a recombinant virus or a self-replicating RNA molecule comprising a polynucleotide encoding one or more polypeptides from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, and wherein the recombinant virus is selected from Ad26, MVA, or GAd20; and

a fourth administration comprising a second composition comprising a recombinant virus or a self-replicating RNA molecule comprising a polynucleotide encoding one or more polypeptides from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, and wherein the recombinant virus is selected from Ad26, MVA, or GAd20.

Embodiment 37. The method of embodiments 34-36, wherein the first, the second, the third or the fourth administration comprise a distinct recombinant virus.

Embodiment 38. The method of embodiments 34-37, wherein the first, the second, the third or the fourth administration comprise a recombinant virus comprising a polynucleotide encoding for a polypeptide of distinct amino acid sequence.

Embodiment 39. The method of any one of embodiments 26-38, further comprising administering a second therapeutic agent selected from a CTLA-4 antibody, a PD-1 antibody, a PD-L1 antibody, a TLR agonist, a CD40 agonist, an OX40 agonist, hydroxyurea, ruxolitinib, fedratinib, a 41BB agonist, a CD28 agonist, FLT3 ligand, aluminum sulfate, a BTK inhibitor, a JAK inhibitor, a CD38 antibody, a CDK inhibitor, a CD33 antibody, a CD37 antibody, a CD25 antibody, a GM-CSF inhibitor, IL-2, IL-15, IL-7, IFNγ, IFNα, TNFα, a VEGF antibody, a CD70 antibody, a CD27 antibody, a BCMA antibody, a GPRC5D antibody, and combinations thereof.

Embodiments 40. The method of embodiment 25-39, wherein multiple myeloma is non-IgM monoclonal gammopathy of undetermined significance (MGUS) or smoldering multiple myeloma (SMM), or a combination thereof.

Embodiment 41. The method of embodiment 23-40, wherein the one or more polypeptides of claim 1 is present at a frequency of at least about 1% or more, about 2% or more, about 3% or more, about 4% or more, about 5% or more, about 6% or more, about 7% or more, about 8% or more, about 9% or more, about 10% or more, about 11% or more, about 12% or more, about 13% or more, about 14% or more, about 15% or more, about 16% or more, about 17% or more, about 18% or more, about 19% or more, about 20% or more, about 21% or more, about 22% or more, about 23% or more, about 24% or more, about 25% or more, about 26% or more, about 27% or more, about 28% or more, about 29% or more, about 30% or more, about 35% or more, about 40% or more, about 45% or more, about 50% or more, about 55% or more, about 60% or more, about 65% or more or about 70% or more in a population of subjects having multiple myeloma.

We claim

1) A polypeptide comprising
a) one or more amino acid sequences selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, or 405, 407, or 421 or fragments thereof;
b) one or more an amino acid sequences that are at least 90% identical to the amino acid sequences selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, or 421 or fragments thereof;
c) one or more amino acid sequences selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof; or
d) one or more an amino acid sequences that are at least 90% identical to the amino acid sequences selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof; or
e) two or more tandem repeats of amino acid sequences of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

2) The polypeptide of claim 1, wherein the one or more amino acid sequences are connected to each other in any order.

3) The polypeptide of claim 2, wherein the one or more amino acid sequences are connected to each other without a linker.

4) The polypeptide of claim 1, wherein the polypeptide comprises one or more reverse peptide bonds, D-isomers of amino acids or chemical modifications, or any combination thereof.

5) A polynucleotide encoding a polypeptide of claim 1.

6) The polynucleotide of claim 5,
a) wherein the polynucleotide is selected from the group consisting of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, or 406, 408, or 422, or fragments thereof;
b) wherein the polynucleotide is selected from the group consisting of SEQ ID NOs: 8, 10, 12, 14, 18, 22, 24, 26, 28, 32, 34, 36, 38, 40, 42, 44, 46, 48, 52, 54, 56, 60, 62, 64, 68, 70, 72, 76, 80, 82, 84, 88, 90, 92, 102, 104, 106, 108, 110, 112, 114, 120, 122, 124, 126, 132, 134, 136, 144, 146, 148, 150, 152, 158, 162, 164, 166, 172, 174, 180, 186, 188, 198, 200, 208, 214, 218, 222, 224, 226, 242, 248, 250, 260, 266, 268, 270, 282, 286, 288, 290, 294, 298, 300, 302, 304, 306, 308, 330, 332, 334, 336, 338, 340, 342, 344, 346, 350, 354, 356, 358, 360, 362, 364, 366, 368, 370, 372, 376, 378, 380, 382, 384, 386, or 422, or fragments thereof;
c) wherein the polynucleotide is at least 90% identical to the polynucleotide of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, or 406, 408, or 422 or fragments thereof; or
d) wherein the polynucleotide is at least 90% identical to the polynucleotide of SEQ ID NOs: 8, 10, 12, 14, 18, 22, 24, 26, 28, 32, 34, 36, 38, 40, 42, 44, 46, 48, 52, 54, 56, 60, 62, 64, 68, 70, 72, 76, 80, 82, 84, 88, 90, 92, 102, 104, 106, 108, 110, 112, 114, 120, 122, 124, 126, 132, 134, 136, 144, 146, 148, 150, 152, 158, 162, 164, 166, 172, 174, 180, 186, 188, 198, 200, 208, 214, 218, 222, 224, 226, 242, 248, 250, 260, 266, 268, 270, 282, 286, 288, 290, 294, 298, 300, 302, 304, 306, 308, 330, 332, 334, 336, 338, 340, 342, 344, 346, 350, 354, 356, 358, 360, 362, 364, 366, 368, 370, 372, 376, 378, 380, 382, 384, 386, or 422, o fragments thereof.

7) The polynucleotide of claim 6, wherein the polynucleotide comprises DNA or RNA

8) The polynucleotide of claim 7, wherein the RNA is mRNA.

9) A vector comprising the polynucleotide of claim 5.

10) The vector of claim 9, wherein the vector is selected from an adenovirus vector, a poxvirus vector, adeno-associated virus vector, a retrovirus vector, a self-replicating RNA molecule, and a combination thereof.

11) The vector of claim 10, wherein the adenovirus vector is derived from hAd5, hAd7, hAd11, hAd26, hAd34, hAd35, hAd48, hAd49, hAd50, GAd20, Gad19, GAd21, GAd25, GAd26, GAd27, GAd28, GAd29, GAd30, GAd31, ChAd3, ChAd4, ChAd5, ChAd6, ChAd7, ChAd8, ChAd9, ChAd10, ChAd 11, ChAdI6, ChAdI7, ChAdI9, ChAd20, ChAd22, ChAd24, ChAd26, ChAd30, ChAd31, ChAd37, ChAd38, ChAd44, ChAd55, ChAd63, ChAd73, ChAd82, ChAd83, ChAd146, ChAd147, PanAdl, PanAd2, or PanAd3.

12) The vector of claim 10, wherein the poxvirus vector is selected from smallpox virus vector, vaccinia virus vector, cowpox virus vector, monkeypox virus vector, Copenhagen vaccinia virus (W) vector, New York Attenuated Vaccinia Virus (NYVAC) vector, and Modified Vaccinia Ankara (MVA) vector.

13) The vector of claim 10, wherein the vector is the adenovirus vector comprising a polynucleotide encoding any one of the polypeptides of claim 1.

14) The vector of claim 10, wherein the vector is the poxvirus vector comprising a polynucleotide encoding any one of the polypeptides of claim 1.

15) The vector of claim 10, wherein the vector is the self-replicating RNA molecule comprising a polynucleotide encoding any one of the polypeptides of claim 1.

16) The vector of claim 11, wherein the vector is an Ad26 vector comprising a polynucleotide
encoding one or more polypeptides selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof; or
encoding one or more polypeptides having at least 90% sequence identity to SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

17) The vector of claim 11, wherein the vector is an GAd20 vector comprising a polynucleotide
encoding one or more polypeptides selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof; or
encoding one or more polypeptides having at least 90% sequence identity to SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

18) The vector of claim 11, wherein the vector is an MVA vector comprising a polynucleotide
encoding one or more polypeptides selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof; or
encoding one or more polypeptides having at least 90% sequence identity to SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

19) The vector of claim 11, wherein the vector is self-replicating RNA molecule comprising a polynucleotide
encoding one or more polypeptides selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, or
encoding one or more polypeptides having at least 90% sequence identity to SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof.

20) A pharmaceutical composition comprising a recombinant virus comprising a vector of claim 9.

21) A method of inducing an immune response in a subject comprising administering to the subject in need thereof a composition comprising a recombinant virus or a self-replicating RNA molecule comprising
a polynucleotide encoding at least one or more polypeptide sequences selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof; or
a polynucleotide encoding one or more polypeptides having at least 90% sequence identity to SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, and wherein the recombinant virus is selected from Ad26, MVA, or GAd20,
and wherein the administration comprises one or more administrations of the composition.

22) A method of treating, preventing, reducing a risk of onset or delaying the onset of multiple myeloma in a subject comprising administering to the subject in need thereof a composition comprising a recombinant virus or a self-replicating RNA molecule comprising
a polynucleotide encoding at least one or more polypeptide sequences selected from the group consisting of SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof; or
a polynucleotide encoding one or more polypeptides having at least 90% sequence identity to SEQ ID NOs: 7, 9, 11, 13, 17, 21, 23, 25, 27, 31, 33, 35, 37, 39, 41, 43, 45, 47, 51, 53, 55, 59, 61, 63, 67, 69, 71, 75, 79, 81, 83, 87, 89, 91, 101, 103, 105, 107, 109, 111, 113, 119, 121, 123, 125, 131, 133, 135, 143, 145, 147, 149, 151, 157, 161, 163, 165, 171, 173, 179, 185, 187, 197, 199, 207, 213, 217, 221, 223, 225, 241, 247, 249, 259, 265, 267, 269, 281, 285, 287, 289, 293, 297, 299, 301, 303, 305, 307, 329, 331, 333, 335, 337, 339, 341, 343, 345, 349, 353, 355, 357, 359, 361, 363, 365, 367, 369, 371, 375, 377, 379, 381, 383, 385, or 421, or fragments thereof, and wherein the virus or recombinant virus is selected from Ad26, MVA, or GAd20;
and wherein the administration comprises one or more administrations of the composition.

23) The method of claim 22, wherein the multiple myeloma is non-IgM monoclonal gammopathy of undetermined significance (MGUS) or smoldering multiple myeloma (SMM), or a combination thereof.

24) The method of claim 22, wherein the multiple myeloma is a relapsed multiple myeloma, or a refractory multiple myeloma.

25) The method of claim 22, wherein the subject has multiple myeloma, is at high risk of multiple myeloma or has one or more chromosomal abnormalities comprising t(4; 14)(p16; q32); t(14; 16)(q32; q23); del17p, t(4; 14)(p16; q32) and t(14; 16)(q32; q23), t(4; 14)(p16; q32) and del17p, t(14; 16)(q32; q23) and del17p; or t(4; 14)(p16; q32), t(14; 16)(q32; q23) and del17p.

26) The method of claim 22, further comprising administering an additional cancer therapeutic agent to the subject.

27) The method of claim 26, wherein the additional cancer therapeutic agent is a chemotherapeutic agent, an immunomodulatory agent, a glutamic acid derivative, a proteasome inhibitor, an alkylating agent, a microtubule inhibitor, a corticosteroid, a radiation therapy, a targeted therapy, a high dose chemotherapy (HDC) and stem cell transplant (SCT), a checkpoint inhibitor, an oncolytic virus, an antibody-drug conjugate or a surgery, or any combination thereof.

28) The method of claim 26, wherein the additional cancer therapeutic agent is a CTLA-4 antibody, a CTLA4 ligand, a PD-1 axis inhibitor, a PD-L1 axis inhibitor, a TLR agonist, a CD40 agonist, an OX40 agonist, hydroxyurea, ruxolitinib, fedratinib, a 41BB agonist, aa CD28 agonist, a STING antagonist, a RIG-1 antagonist, TCR-T therapy, CAR-T therapy, FLT3 ligand, aluminum sulfate, BTK inhibitor, CD38 antibody, CDK inhibitor, CD33 antibody, CD37 antibody, CD25 antibody, GM-CSF inhibitor, IL-2, IL-15, IL-7, CD3 redirection molecules, pomalimib, IFNγ, IFNα, TNFα, VEGF antibody, CD70 antibody, CD27 antibody, BCMA antibody or GPRC5D antibody, or any combination thereof.

29) The method of claim 27, wherein the checkpoint inhibitor is ipilimumab, cetrelimab, pembrolizumab, nivolumab, sintilimab. cemiplimab, toripalimab, camrelizumab, tislelizumab, dostralimab, spartalizumab, prolgolimab, balstilimab, budigalimab, sasanlimab, avelumab, atezolizumab, durvalumab, envafolimab, or iodapolimab, or any combination thereof.


SEQUENCE LISTING (425 sequences)

SEQ ID NO: 1 - protein, 11 aa, Artificial Sequence
  Description of Artificial Sequence Synthetic peptide
ISNKIALLQTF

SEQ ID NO: 2 - DNA, 33 bp, Artificial Sequence
  Description of Artificial Sequence Synthetic oligonucleotide
atatctaataagatagccttactgcaaaccttt

SEQ ID NO: 3 - protein, 31 aa, Artificial Sequence
  Description of Artificial Sequence Synthetic polypeptide
WLVKQRGWANLLEKSAEQICTGEFKAKASQS

SEQ ID NO: 4 - DNA, 93 bp, Artificial Sequence
  Description of Artificial Sequence Synthetic oligonucleotide
tggctagttaaacaaagaggctgggcaaaccttttagagaagtcagctgagcaaatatgtacaggtgaattcaaagcaaaagcctcacaaagt

SEQ ID NO: 5 - protein, 15 aa, Artificial Sequence
  Description of Artificial Sequence Synthetic peptide
NHETAFQGKPFREVS

SEQ ID NO: 6 - DNA, 45 bp, Artificial Sequence
  Description of Artificial Sequence Synthetic oligonucleotide
aaccacgagacggccttccaaggcaaaccttttagagaagtcagc

SEQ ID NO: 7 - protein, 15 aa, Artificial Sequence
  Description of Artificial Sequence Synthetic peptide
DVAVICSGRRSARDP

SEQ ID NO: 8 - DNA, 45 bp, Artificial Sequence
  Description of Artificial Sequence Synthetic oligonucleotide
gatgtggctgtcatctgctcaggaagacgttcagccagggatcca

SEQ ID NO: 9 - protein, 15 aa, Artificial Sequence
  Description of Artificial Sequence Synthetic peptide
DVAVICSGVPVADVS

SEQ ID NO: 10 - DNA, 45 bp, Artificial Sequence
  Description of Artificial Sequence Synthetic oligonucleotide
gatgtggctgtcatctgctcaggggtccctgtcgctgatgtgagc

SEQ ID NO: 11 - protein, 16 aa, Artificial Sequence
  Description of Artificial Sequence Synthetic peptide
EDVAVICSELFLIASP

SEQ ID NO: 12 - DNA, 48 bp, Artificial Sequence
  Description of Artificial Sequence Synthetic oligonucleotide
gaagatgtggctgtcatctgctcagagctgtttttgatagccagcccc

SEQ ID NO: 13 - protein, 18 aa, Artificial Sequence
  Description of Artificial Sequence Synthetic peptide
GRAGQGGGMISVSQEFIK

SEQ ID NO: 14 - DNA, 54 bp, Artificial Sequence
  Description of Artificial Sequence Synthetic oligonucleotide
ggtcgtgcaggccagggaggaggcatgatttctgtgagtcaggaattcattaag

SEQ ID NO: 15 - protein, 24 aa, Artificial Sequence
  Description of Artificial Sequence Synthetic peptide
AVLKLTDQHNAVKMGEERCVSSEI

SEQ ID NO: 16 - DNA, 72 bp, Artificial Sequence
  Description of Artificial Sequence Synthetic oligonucleotide
gcagtgctcaaactcaccgaccagcataatgcagtgaagatgggtgaagagagatgtgtttcatctgaaatt

SEQ ID NO: 17 - protein, 17 aa, Artificial Sequence
  Description of Artificial Sequence Synthetic peptide
SGLLMNTLTVLILYFLY

SEQ ID NO: 18 - DNA, 51 bp, Artificial Sequence
  Description of Artificial Sequence Synthetic oligonucleotide
agtggccttctgatgaatactttaactgtgctgatcctgtatttcctgtat

SEQ ID NO: 19 - protein, 14 aa, Artificial Sequence
  Description of Artificial Sequence Synthetic peptide
APKPKKMKNHLLFW

SEQ ID NO: 20 - DNA, 42 bp, Artificial Sequence
  Description of Artificial Sequence Synthetic oligonucleotide
gctcctaagcccaagaagatgaagaaccatttgcttttctgg

SEQ ID NO: 21 - protein, 16 aa, Artificial Sequence
  Description of Artificial Sequence Synthetic peptide
GKSTPPRKEIFDDASP

SEQ ID NO: 22 - DNA, 48 bp, Artificial Sequence
  Description of Artificial Sequence Synthetic oligonucleotide
gggaaatccacaccaccacgtaaggaaatatttgatgatgcgtcacct

SEQ ID NO: 23 - protein, 12 aa, Artificial Sequence
  Description of Artificial Sequence Synthetic peptide
ARWFTREQGSEN

SEQ ID NO: 24 - DNA, 36 bp, Artificial Sequence
  Description of Artificial Sequence Synthetic oligonucleotide
gcccgctggttcactagagaacagggcagtgaaaac

SEQ ID NO: 25 - protein, 19 aa, Artificial Sequence
  Description of Artificial Sequence Synthetic peptide
AFIYHAFVGSTKTAWRTTL

SEQ ID NO: 26 - DNA, 57 bp, Artificial Sequence
  Description of Artificial Sequence Synthetic oligonucleotide
gcgttcatctaccacgctttcgttgggtcaaccaaaaccgcatggcgtactactctc

SEQ ID NO: 27 - protein, 11 aa, Artificial Sequence
  Description of Artificial Sequence Synthetic peptide
LARQISFKSLI

SEQ ID NO: 28 - DNA, 33 bp, Artificial Sequence
  Description of Artificial Sequence Synthetic oligonucleotide
ctggctaggcaaataagctttaagtcacttata

SEQ ID NO: 29 - protein, 18 aa, Artificial Sequence
  Description of Artificial Sequence Synthetic peptide
QPRDNHTDQVTYIRRTLM

SEQ ID NO: 30 - DNA, 54 bp, Artificial Sequence
  Description of Artificial Sequence Synthetic oligonucleotide
cagccaagagacaaccacactgatcaagtcacttatataagaagaaccttgatg

SEQ ID NO: 31 - protein, 34 aa, Artificial Sequence
  Description of Artificial Sequence Synthetic polypeptide
PRDNHTDHENPRGSDGQGRWKCPSQVTYIRRTLM

SEQ ID NO: 32 - DNA, 102 bp, Artificial Sequence
  Description of Artificial Sequence Synthetic polynucleotide
ccaagagacaaccacactgatcatgaaaacccaagaggaagcgatggacagggcaggtggaaatgcccaagccaagtcacttatataagaagaaccttgatg

SEQ ID NO: 33 - protein, 14 aa, Artificial Sequence
  Description of Artificial Sequence Synthetic peptide
AAGVVTEIEAAVKA

SEQ ID NO: 34 - DNA, 42 bp, Artificial Sequence
  Description of Artificial Sequence Synthetic oligonucleotide
gctgctggtgttgtcactgagatcgaagccgcggtcaaggcc

SEQ ID NO: 35 - protein, 16 aa, Artificial Sequence
  Description of Artificial Sequence Synthetic peptide
NAVRSYYEGTENIVAV

SEQ ID NO: 36 - DNA, 48 bp, Artificial Sequence
  Description of Artificial Sequence Synthetic oligonucleotide
aacgcagttcggagttattatgaggggacggaaaatatagtcgcggtg

SEQ ID NO: 37 - protein, 20 aa, Artificial Sequence
  Description of Artificial Sequence Synthetic peptide
NAVRSYYETNGGMSFLRITP

SEQ ID NO: 38 - DNA, 60 bp, Artificial Sequence
  Description of Artificial Sequence Synthetic oligonucleotide
aacgcagttcggagttattatgagacaaatggaggcatgagcttccttagaattacccct

SEQ ID NO: 39 - protein, 50 aa, Artificial Sequence
  Description of Artificial Sequence Synthetic polypeptide
ISKRLKSRRRSGWQLNRAGNRGLSPGLGLFPRGCCRWGGAYTRLPSANQT

SEQ ID NO: 40 - DNA, 150 bp, Artificial Sequence
  Description of Artificial Sequence Synthetic polynucleotide
atcagcaagcgactcaagagcagaaggagatccggttggcagctaaaccgcgctgggaacaggggcctgagtcctggactagggctctttccccggggctgctgcagatggggaggagcctacacccgcctcccgagtgctaatcagacc

SEQ ID NO: 41 - protein, 15 aa, Artificial Sequence
  Description of Artificial Sequence Synthetic peptide
PPTAAASQTCLELER

SEQ ID NO: 42 - DNA, 45 bp, Artificial Sequence
  Description of Artificial Sequence Synthetic oligonucleotide
ccacccaccgccgccgcctcccagacctgcctagagctggaacgt

SEQ ID NO: 43 - protein, 25 aa, Artificial Sequence
  Description of Artificial Sequence Synthetic peptide
ACGACGAGFFIKQKCIEQRESRSLS

SEQ ID NO: 44 - DNA, 75 bp, Artificial Sequence
  Description of Artificial Sequence Synthetic oligonucleotide
gcctgcggagcctgtggggcaggattttttatcaagcagaaatgcatcgaacaacgagaatcaagatcactgagc

SEQ ID NO: 45 - protein, 84 aa, Artificial Sequence
  Description of Artificial Sequence Synthetic polypeptide
VYPVYQPVVSQAGLGELWQWASGKLRGYCWRRGQWHAYSIQCDPHPVFGEGACDCSETEAHGGCQSPRLESFPGQPPGFRGQRF

SEQ ID NO: 46 - DNA, 252 bp, Artificial Sequence
  Description of Artificial Sequence Synthetic polynucleotide
gtgtaccctgtctaccagcctgtggtttcacaagctggtctgggggagctttggcagtgggcttctggaaagctccggggttattgttggcggcggggacaatggcatgcttattctatacaatgtgacccacatcctgtcttcggggaaggagcctgtgattgctcagaaacagaagcacacgggggctgtcagagccctcgacttgaatcctttccagggcaacctcctggcttcaggggccagcgattc

SEQ ID NO: 47 - protein, 16 aa, Artificial Sequence
  Description of Artificial Sequence Synthetic peptide
SGLEAIQRGYETFYSE

SEQ ID NO: 48 - DNA, 48 bp, Artificial Sequence
  Description of Artificial Sequence Synthetic oligonucleotide
tctggcctggaggctatccagcggggatatgagactttctactcggaa

SEQ ID NO: 49 - protein, 16 aa, Artificial Sequence
  Description of Artificial Sequence Synthetic peptide
SPEDYTLKAQGPGQCP

SEQ ID NO: 50 - DNA, 48 bp, Artificial Sequence
  Description of Artificial Sequence Synthetic oligonucleotide
tccccagaggactacacgctcaaggcccagggccccggccagtgccca

SEQ ID NO: 51 - protein, 15 aa, Artificial Sequence
  Description of Artificial Sequence Synthetic peptide
ARHLLTLGTCGSTWP

SEQ ID NO: 52 - DNA, 45 bp, Artificial Sequence
  Description of Artificial Sequence Synthetic oligonucleotide
gcgaggcacttgctaactcttgggacatgcggaagcacgtggcca

SEQ ID NO: 53 - protein, 19 aa, Artificial Sequence
  Description of Artificial Sequence Synthetic peptide
PQEEVPFRSITTELFPSMC

SEQ ID NO: 54 - DNA, 57 bp, Artificial Sequence
  Description of Artificial Sequence Synthetic oligonucleotide
ccacaggaagaagtgccattcaggtcaataacaacggagttgtttccttcaatgtgc

SEQ ID NO: 55 - protein, 16 aa, Artificial Sequence
  Description of Artificial Sequence Synthetic peptide
QIIYDENRTMQIFVKT

SEQ ID NO: 56 - DNA, 48 bp, Artificial Sequence
  Description of Artificial Sequence Synthetic oligonucleotide
cagatcatctacgacgagaaccggacaatgcagatcttcgtgaagact

SEQ ID NO: 57 - protein, 16 aa, Artificial Sequence
  Description of Artificial Sequence Synthetic peptide
GERERREKTMQIFVKT

SEQ ID NO: 58 - DNA, 48 bp, Artificial Sequence
  Description of Artificial Sequence Synthetic oligonucleotide
ggcgagcgcgagcggcgcgagaaaacaatgcagatcttcgtgaagact

SEQ ID NO: 59 - protein, 16 aa, Artificial Sequence
  Description of Artificial Sequence Synthetic peptide
MDLTAIYETMQIFVKT

SEQ ID NO: 60 - DNA, 48 bp, Artificial Sequence
  Description of Artificial Sequence Synthetic oligonucleotide
atggatctgactgccatctacgagacaatgcagatcttcgtgaagact

SEQ ID NO: 61 - protein, 16 aa, Artificial Sequence
  Description of Artificial Sequence Synthetic peptide
PPTAAASQTMQIFVKT

SEQ ID NO: 62 - DNA, 48 bp, Artificial Sequence
  Description of Artificial Sequence Synthetic oligonucleotide
ccacccaccgccgccgcctcccagacaatgcagatcttcgtgaagact

SEQ ID NO: 63 - protein, 18 aa, Artificial Sequence
  Description of Artificial Sequence Synthetic peptide
SSVTTHSSGNLRESPIYH

SEQ ID NO: 64 - DNA, 54 bp, Artificial Sequence
  Description of Artificial Sequence Synthetic oligonucleotide
tcttctgtgactacccactcatctggaaatttgcgagaatccccgatttatcat

SEQ ID NO: 65 - protein, 16 aa, Artificial Sequence
  Description of Artificial Sequence Synthetic peptide
SGLEAIQREKPYHCNW

SEQ ID NO: 66 - DNA, 48 bp, Artificial Sequence
  Description of Artificial Sequence Synthetic oligonucleotide
tctggcctggaggctatccagcgtgagaagccctaccactgcaactgg

SEQ ID NO: 67 - protein, 10 aa, Artificial Sequence
  Description of Artificial Sequence Synthetic peptide
VAPSGLYYCG

SEQ ID NO: 68 - DNA, 30 bp, Artificial Sequence
  Description of Artificial Sequence Synthetic oligonucleotide
gtggcacccagtggcctgtactactgtgga

SEQ ID NO: 69 - protein, 9 aa, Artificial Sequence
  Description of Artificial Sequence Synthetic peptide
NRVTALTQN

SEQ ID NO: 70 - DNA, 27 bp, Artificial Sequence
  Description of Artificial Sequence Synthetic oligonucleotide
aacagggtcacagctctcacccagaac

SEQ ID NO: 71 - protein, 12 aa, Artificial Sequence
  Description of Artificial Sequence Synthetic peptide
IEKSISARITEQ

SEQ ID NO: 72 - DNA, 36 bp, Artificial Sequence
  Description of Artificial Sequence Synthetic oligonucleotide
atagagaaatcaatttctgctaggatcacagaacaa

SEQ ID NO: 73 - protein, 64 aa, Artificial Sequence
  Description of Artificial Sequence Synthetic polypeptide
PSDCSIRKTLLSLPGGVETGTRRSCLTRRPRPGSVRDSRDLWLQTPPARRPRAGALLRMRLWGP

SEQ ID NO: 74 - DNA, 192 bp, Artificial Sequence
  Description of Artificial Sequence Synthetic polynucleotide
ccaagtgactgctccattcggaagactttgttgtcgctgcccggaggagtcgagactggtacccggaggagctgtctcaccaggagaccacgtcctggaagtgtccgggactcgcgggacctgtggctgcagaccccgccggcacgcaggcccagagctggcgcactcctgaggatgagactctgggggccc

SEQ ID NO: 75 - protein, 15 aa, Artificial Sequence
  Description of Artificial Sequence Synthetic peptide
GEVKWSVGSRTSLLL

SEQ ID NO: 76 - DNA, 45 bp, Artificial Sequence
  Description of Artificial Sequence Synthetic oligonucleotide
ggggaagtgaagtggtctgtaggctcccggacgtccctgctcctg

SEQ ID NO: 77 - protein, 61 aa, Artificial Sequence
  Description of Artificial Sequence Synthetic polypeptide
LTEKKASDVRSPTTATGTAAAGSLRAQTSSRATTESTRATGHSSAICAIVPSRAPITWRCT

SEQ ID NO: 78 - DNA, 183 bp, Artificial Sequence
  Description of Artificial Sequence Synthetic polynucleotide
ctcacggagaagaaggcctccgacgtgagaagccctaccactgcaactgggacggctgcggctggaagtttgcgcgctcagacgagctcacgcgccactaccgaaagcacacgggccaccggccattccagtgccatctgtgcgatcgtgccttctcgcgctccgatcacctggcgctgcaca

SEQ ID NO: 79 - protein, 16 aa, Artificial Sequence
  Description of Artificial Sequence Synthetic peptide
CRERGLQEAEGDVAAL

SEQ ID NO: 80 - DNA, 48 bp, Artificial Sequence
  Description of Artificial Sequence Synthetic oligonucleotide
tgccgcgagcgcggcctgcaggaggctgaaggtgatgtggccgccctc

SEQ ID NO: 81 - protein, 61 aa, Artificial Sequence
  Description of Artificial Sequence Synthetic polypeptide
PPTAAASQVRSPTTATGTAAAGSLRAQTSSRATTESTRATGHSSAICAIVPSRAPITWRCT

SEQ ID NO: 82 - DNA, 183 bp, Artificial Sequence
  Description of Artificial Sequence Synthetic polynucleotide
ccacccaccgccgccgcctcccaggtgagaagccctaccactgcaactgggacggctgcggctggaagtttgcgcgctcagacgagctcacgcgccactaccgaaagcacacgggccaccggccattccagtgccatctgtgcgatcgtgccttctcgcgctccgatcacctggcgctgcaca

SEQ ID NO: 83 - protein, 38 aa, Artificial Sequence
  Description of Artificial Sequence Synthetic polypeptide
GGPPFVGPLRGPRAQPSLSGLCGRRTGSRDTIKIPGQT

SEQ ID NO: 84 - DNA, 114 bp, Artificial Sequence
  Description of Artificial Sequence Synthetic polynucleotide
ggtggccctccttttgtaggcccgcttcgggggccgcgtgcccagcctagcctatccggactgtgcgggaggcggacggggagcagagacacaataaaaattcccggtcaaacc

SEQ ID NO: 85 - protein, 32 aa, Artificial Sequence
  Description of Artificial Sequence Synthetic polypeptide
GLKQLHKAGYEEETQNNRSGLWNYMELWQAAL

SEQ ID NO: 86 - DNA, 96 bp, Artificial Sequence
  Description of Artificial Sequence Synthetic oligonucleotide
ggtttgaaacagcttcacaaggcaggttatgaagaagaaactcaaaataacaggagtggcttatggaactacatggagctctggcaggctgccctg

SEQ ID NO: 87 - protein, 15 aa, Artificial Sequence
  Description of Artificial Sequence Synthetic peptide
LDQCVETLCSLSKSD

SEQ ID NO: 88 - DNA, 45 bp, Artificial Sequence
  Description of Artificial Sequence Synthetic oligonucleotide
ctggaccagtgtgtggagaccctctgctccctcagcaagtcggac

SEQ ID NO: 89 - protein, 17 aa, Artificial Sequence
  Description of Artificial Sequence Synthetic peptide
LLPPGQESAIIGPPGMQ

SEQ ID NO: 90 - DNA, 51 bp, Artificial Sequence
  Description of Artificial Sequence Synthetic oligonucleotide
ctccttccacctggccaggaatcagccattattggaccccctggaatgcaa

SEQ ID NO: 91 - protein, 16 aa, Artificial Sequence
  Description of Artificial Sequence Synthetic peptide
YECTGCKTSFPHRSQS

SEQ ID NO: 92 - DNA, 48 bp, Artificial Sequence
  Description of Artificial Sequence Synthetic oligonucleotide
tatgaatgtactggatgcaaaacgagcttcccacacagaagccagagc

SEQ ID NO: 93 - protein, 12 aa, Artificial Sequence
  Description of Artificial Sequence Synthetic peptide
QHPARLVKCGTQ

SEQ ID NO: 94 - DNA, 36 bp, Artificial Sequence
  Description of Artificial Sequence Synthetic oligonucleotide
caacatcctgctagacttgttaagtgtggaacacaa

SEQ ID NO: 95 - protein, 56 aa, Artificial Sequence
  Description of Artificial Sequence Synthetic polypeptide
GLTLGARTTLTHGSPGPSQATVAVAPLSGRAAVSAAGGCVFLLLPACVPRRCHWIP

SEQ ID NO: 96 - DNA, 168 bp, Artificial Sequence
  Description of Artificial Sequence Synthetic polynucleotide
ggcctgactctgggagcacgcactactctgacccatggatcccctgggcccagccaagccacagtggctgtggcgccgctgtctggccgggctgctgtttcagctgctggtggctgtgtgtttcttctcctacctgcgtgtgtcccgagacgatgccactggatcccc

SEQ ID NO: 97 - protein, 16 aa, Artificial Sequence
  Description of Artificial Sequence Synthetic peptide
MDLTAIYECCNLSVSE

SEQ ID NO: 98 - DNA, 48 bp, Artificial Sequence
  Description of Artificial Sequence Synthetic oligonucleotide
atggatctgactgccatctacgagtgctgcaacctgagcgtgagcgag

SEQ ID NO: 99 - protein, 17 aa, Artificial Sequence
  Description of Artificial Sequence Synthetic peptide
VIVDPIQGYLRQWQLLD

SEQ ID NO: 100 - DNA, 51 bp, Artificial Sequence
  Description of Artificial Sequence Synthetic oligonucleotide
gtaattgtggaccctatacagggatacctcagacaatggcagcttttggat

SEQ ID NO: 101 - protein, 27 aa, Artificial Sequence
  Description of Artificial Sequence Synthetic peptide
PPLITNVTEKPWKHTRIEMPLARLTRP

SEQ ID NO: 102 - DNA, 81 bp, Artificial Sequence
  Description of Artificial Sequence Synthetic oligonucleotide
ccgccactcatcaccaatgtcacggaaaagccgtggaaacacacccggattgaaatgcccctggcccgcctgacccgaccc

SEQ ID NO: 103 - protein, 20 aa, Artificial Sequence
  Description of Artificial Sequence Synthetic peptide
MEPTTKRGPGGTMVSAAAPS

SEQ ID NO: 104 - DNA, 60 bp, Artificial Sequence
  Description of Artificial Sequence Synthetic oligonucleotide
atggaacctactaccaaaagaggtcctgggggcaccatggtttcggcggcagcccccagc

SEQ ID NO: 105 - protein, 9 aa, Artificial Sequence
  Description of Artificial Sequence Synthetic peptide
RPGVFTDIL

SEQ ID NO: 106 - DNA, 27 bp, Artificial Sequence
  Description of Artificial Sequence Synthetic oligonucleotide
aggcctggagtattcacagacattctt

SEQ ID NO: 107 - protein, 17 aa, Artificial Sequence
  Description of Artificial Sequence Synthetic peptide
MDLTAIYEDSAGPLSHA

SEQ ID NO: 108 - DNA, 51 bp, Artificial Sequence
  Description of Artificial Sequence Synthetic oligonucleotide
atggatctgactgccatctacgaggactctgctgggccgctcagccatgcc

SEQ ID NO: 109 - protein, 13 aa, Artificial Sequence
  Description of Artificial Sequence Synthetic peptide
LRYLIHLRKWKIK

SEQ ID NO: 110 - DNA, 39 bp, Artificial Sequence
  Description of Artificial Sequence Synthetic oligonucleotide
ttacgctacctcatacatctgaggaaatggaagataaag

SEQ ID NO: 111 - protein, 15 aa, Artificial Sequence
  Description of Artificial Sequence Synthetic peptide
FALMASLGIPQTMAA

SEQ ID NO: 112 - DNA, 45 bp, Artificial Sequence
  Description of Artificial Sequence Synthetic oligonucleotide
tttgcacttatggcctctctggggatacctcagacaatggcagct

SEQ ID NO: 113 - protein, 43 aa, Artificial Sequence
  Description of Artificial Sequence Synthetic polypeptide
RQLQQQLSSLSGPEGVPFLKLWCLDDLLSTLLGTCRCPSRAQF

SEQ ID NO: 114 - DNA, 129 bp, Artificial Sequence
  Description of Artificial Sequence Synthetic polynucleotide
cggcaacttcaacaacagctctctagcctgtctggccctgagggagtcccctttctgaagctgtggtgcttggacgacctgctctctacattgctgggcacctgtaggtgtccctcgagagctcagttt

SEQ ID NO: 115 - protein, 24 aa, Artificial Sequence
  Description of Artificial Sequence Synthetic peptide
LKTTNRASVSISQLPFFKIIFLWS

SEQ ID NO: 116 - DNA, 72 bp, Artificial Sequence
  Description of Artificial Sequence Synthetic oligonucleotide
ctgaagacaacaaatagagcctctgtaagtattagtcagttgcctttctttaagattatttttctgtggtct

SEQ ID NO: 117 - protein, 16 aa, Artificial Sequence
  Description of Artificial Sequence Synthetic peptide
RLASIYEELLGATAIE

SEQ ID NO: 118 - DNA, 48 bp, Artificial Sequence
  Description of Artificial Sequence Synthetic oligonucleotide
aggctggctagcatctatgaggagctgctgggtgcaacggccattgag

SEQ ID NO: 119 - protein, 21 aa, Artificial Sequence
  Description of Artificial Sequence Synthetic peptide
SLLSLFPGKTVEITCLQHQKC

SEQ ID NO: 120 - DNA, 63 bp, Artificial Sequence
  Description of Artificial Sequence Synthetic oligonucleotide
agtcttcttagtctcttcccaggtaagactgttgaaataacatgcctgcagcatcaaaaatgc

SEQ ID NO: 121 - protein, 113 aa, Artificial Sequence
  Description of Artificial Sequence Synthetic polypeptide
TQRLPGHKVSNSCMTGLVLTFTKRDPFHKPVTWCGQLADVRQCMFHSIRERAHPLQRALATLNFFFVSFYTALYLTHPLLTLQTWEVVVTVCFSCWELCVPCDVLRRTCRGQR

SEQ ID NO: 122 - DNA, 339 bp, Artificial Sequence
  Description of Artificial Sequence Synthetic polynucleotide
actcaacggttgccaggacacaaagtaagcaacagctgcatgaccggtttagtcctgacgtttacaaagagggaccctttccataagcctgtaacttggtgtgggcagcttgccgatgtcaggcagtgcatgtttcactcgattagggagagagcgcaccctctccagagggctttggccacgcttaattttttctttgtttccttctatactgctttatatctcacacatcccctcttaactctccagacatgggaagttgttgtgacagtatgttttagctgttgggagctctgtgttccatgcgatgttttacggagaacttgcagaggacaaaga

SEQ ID NO: 123 - protein, 16 aa, Artificial Sequence
  Description of Artificial Sequence Synthetic peptide
LQAILQPGNFILVNEI

SEQ ID NO: 124 - DNA, 48 bp, Artificial Sequence
  Description of Artificial Sequence Synthetic oligonucleotide
ttgcaggcaattttgcaaccaggtaacttcattctggtcaacgagatt

SEQ ID NO: 125 - protein, 102 aa, Artificial Sequence
  Description of Artificial Sequence Synthetic polypeptide
RQREVVDLVSPREPSRDRRAGGGDLAARPTGRASVWLLGVSVCTAVCRVSCCAGVSCFLFTGEEGPGYACSESWNSEGRPKWEAIVERTCDMGPYNGMCLQG

SEQ ID NO: 126 - DNA, 306 bp, Artificial Sequence
  Description of Artificial Sequence Synthetic polynucleotide
cggcagagggaggtggtggacctggtgagtccgagggagccgagccgggaccgccgcgctggtggaggggacctggccgcgcgccccacgggcagggcgtcagtctggctgttgggggtgtctgtctgtacagctgtgtgtcgtgtgtcttgctgcgccggggtgtcatgctttttatttacaggtgaagaaggacctggttatgcgtgcagtgagtcttggaactcagaggggagaccaaaatgggaggccatcgtggagcggacatgcgatatgggcccgtataatggaatgtgcttacaaggg

SEQ ID NO: 127 - protein, 23 aa, Artificial Sequence
  Description of Artificial Sequence Synthetic peptide
LVHQLKQQVGPPARQTQNRMKLM

SEQ ID NO: 128 - DNA, 69 bp, Artificial Sequence
  Description of Artificial Sequence Synthetic oligonucleotide
ctagtacatcaattaaagcagcaggtggggcctcctgcaagacagacccagaacagaatgaagttgatg

SEQ ID NO: 129 - protein, 23 aa, Artificial Sequence
  Description of Artificial Sequence Synthetic peptide
FHLIAMDAVSFFYFQIFITSRIF

SEQ ID NO: 130 - DNA, 69 bp, Artificial Sequence
  Description of Artificial Sequence Synthetic oligonucleotide
tttcatctcatagctatggatgctgtatcctttttttatttccagatttttataactagtagaatattt

SEQ ID NO: 131 - protein, 30 aa, Artificial Sequence
  Description of Artificial Sequence Synthetic polypeptide
GEKILFIRVKFYFSLILFLWEDSEFELGEI

SEQ ID NO: 132 - DNA, 90 bp, Artificial Sequence
  Description of Artificial Sequence Synthetic oligonucleotide
ggagaaaagatcctcttcattagggtaaagttttacttttcacttatcttgtttttatgggaagattcagaatttgagttaggggaaata

SEQ ID NO: 133 - protein, 54 aa, Artificial Sequence
  Description of Artificial Sequence Synthetic polypeptide
MKQFLDFGECGPGPWAFLRGPGRGAAAAAPGRVGAGQLSPEAHPSSSAFPPPAP

SEQ ID NO: 134 - DNA, 162 bp, Artificial Sequence
  Description of Artificial Sequence Synthetic polynucleotide
atgaagcagttcctggacttcggtgagtgcggcccgggaccttgggcctttttgcgcggtcccgggcggggagctgcggccgctgccccaggccgggtcggcgccggccagctctcgcctgaggcgcacccctcctcctcagcgtttccgcccccagcgcct

SEQ ID NO: 135 - protein, 138 aa, Artificial Sequence
  Description of Artificial Sequence Synthetic polypeptide
QLKKMVSKVRLRRARLPGRAHRSLPRPVESVPAQPSKLPRRAGSPGAVLSDLLRTPPRGACRLVRESPYGSWEGGGRRQSTKAWSGGYLTPAAHPPSSLPLSLELSVGQCSFSFVFINTNKGSYLVVDLRQSSTPVPI

SEQ ID NO: 136 - DNA, 414 bp, Artificial Sequence
  Description of Artificial Sequence Synthetic polynucleotide
cagctcaagaaaatggtgtccaaggtgaggctgcgacgcgctcgcctcccagggcgcgcccaccgctcccttccgcgccctgtcgagtccgtcccggcccagccaagcaagcttcccagacgggccggaagccccggtgcagtccttagcgacctcctcagaaccccgccccgaggcgcctgtcgcctggtgcgggaatccccgtacgggagctgggagggtgggggacggcgacagtcaacaaaggcgtggagcggaggctacctgacacctgccgcccacccgccctcctctcttccactgagtttggagctgtctgtggggcagtgtagtttttcgtttgtttttataaacacaaacaagggatcatacttagttgtagatctgaggcaatcctctactcctgtcccaatc

SEQ ID NO: 137 - protein, 26 aa, Artificial Sequence
  Description of Artificial Sequence Synthetic peptide
DALIQNQHVSTVVCIVSFRIDKLLKY

SEQ ID NO: 138 - DNA, 78 bp, Artificial Sequence
  Description of Artificial Sequence Synthetic oligonucleotide
gatgctttaatacagaaccagcatgtgagtacagtagtttgtattgtttcatttagaattgataaacttttaaaatat

SEQ ID NO: 139 - protein, 16 aa, Artificial Sequence
  Description of Artificial Sequence Synthetic peptide
GGNNSNQQLDDARNKA

SEQ ID NO: 140 - DNA, 48 bp, Artificial Sequence
  Description of Artificial Sequence Synthetic oligonucleotide
ggaggaaataattcaaatcagcaactggatgatgctcgcaacaaagct

SEQ ID NO: 141 - protein, 83 aa, Artificial Sequence
  Description of Artificial Sequence Synthetic polypeptide
NSGHLLVQRWSLALSPRPECSGMISAHCNLHLPGSSSSPASASQVDGITGTCRHFFNDVFVNGKETAIFYLWGFLPIKELPAA

SEQ ID NO: 142 - DNA, 249 bp, Artificial Sequence
  Description of Artificial Sequence Synthetic polynucleotide
aattcgggccacttgctagtccagagatggagtcttgctctgtcgcccaggccagagtgcagtggcatgatctcagctcactgcaacctccacctcccgggttcaagcagttctcctgcctcagcctcccaagtagatgggattacaggcacgtgccgccacttttttaatgatgtatttgttaatgggaaggagacagctattttctacttatggggcttccttcctataaaagaattgccagcagca

SEQ ID NO: 143 - protein, 41 aa, Artificial Sequence
  Description of Artificial Sequence Synthetic polypeptide
YGVYVINQKGHSGCIWSMDGEGRLETAWPVWKRGRCPGKRR

SEQ ID NO: 144 - DNA, 123 bp, Artificial Sequence
  Description of Artificial Sequence Synthetic polynucleotide
tacggcgtctatgtcatcaaccagaaaggtcactctggttgcatatggagcatggatggagaggggagattagagacagcgtggcccgtttggaagagaggacgatgcccaggaaagaggcgg

SEQ ID NO: 145 - protein, 40 aa, Artificial Sequence
  Description of Artificial Sequence Synthetic polypeptide
SRAPIKMSVLGQGHSRCLLHCLCLVAPMAFPVRLRVPCGG

SEQ ID NO: 146 - DNA, 120 bp, Artificial Sequence
  Description of Artificial Sequence Synthetic polynucleotide
agtcgcgcacccataaaaatgagcgttctgggtcagggccattctcggtgcctcctgcactgcctttgtttggttgctcccatggcgttcccggtgagactgcgggtgccgtgcggtggt

SEQ ID NO: 147 - protein, 20 aa, Artificial Sequence
  Description of Artificial Sequence Synthetic peptide
SATTRSITGQDILSLEKERR

SEQ ID NO: 148 - DNA, 60 bp, Artificial Sequence
  Description of Artificial Sequence Synthetic oligonucleotide
agtgccaccaccaggtctataaccggacaagatatcttgagcctggagaaggaaagaaga

SEQ ID NO: 149 - protein, 13 aa, Artificial Sequence
  Description of Artificial Sequence Synthetic peptide
LTALLLLASTPTP

SEQ ID NO: 150 - DNA, 39 bp, Artificial Sequence
  Description of Artificial Sequence Synthetic oligonucleotide
ctgacagcccttctgctcctggcttccactccaaccccc

SEQ ID NO: 151 - protein, 17 aa, Artificial Sequence
  Description of Artificial Sequence Synthetic peptide
FSMRPAFGETALGCKKW

SEQ ID NO: 152 - DNA, 51 bp, Artificial Sequence
  Description of Artificial Sequence Synthetic oligonucleotide
ttcagcatgagacccgccttcggagagacagccctgggatgtaaaaagtgg

SEQ ID NO: 153 - protein, 17 aa, Artificial Sequence
  Description of Artificial Sequence Synthetic peptide
SSLRSLWGSRLLLQPSP

SEQ ID NO: 154 - DNA, 51 bp, Artificial Sequence
  Description of Artificial Sequence Synthetic oligonucleotide
tccagcctgcggagcctgtggggcagcaggctcctgctccagcccagcccc

SEQ ID NO: 155 - protein, 21 aa, Artificial Sequence
  Description of Artificial Sequence Synthetic peptide
QLPPMQERATISLQGTEYPKY

SEQ ID NO: 156 - DNA, 63 bp, Artificial Sequence
  Description of Artificial Sequence Synthetic oligonucleotide
caacttccacccatgcaggaaagagctacgatatccttacaaggtaccgaatatcccaaatat

SEQ ID NO: 157 - protein, 76 aa, Artificial Sequence
  Description of Artificial Sequence Synthetic polypeptide
TFTHLKQQLSLLPLMEPIIGVNFAHFLPYGSGQFNSGNRLLGTFGSATLEGVSDYYSQLIYKVCFSAKVLSCFIFG

SEQ ID NO: 158 - DNA, 228 bp, Artificial Sequence
  Description of Artificial Sequence Synthetic polynucleotide
acgtttacccacttgaaacagcagctctctctgctccctctaatggaaccaatcattggagtgaactttgcgcactttcttccttatggcagtggccaatttaatagtgggaatcgacttctaggaacttttggcagtgctaccctggaaggggtttcggactactattctcagttgatctacaaggtatgtttctctgccaaggtgttgtcttgcttcatctttggg

SEQ ID NO: 159 - protein, 16 aa, Artificial Sequence
  Description of Artificial Sequence Synthetic peptide
CKVCQNCKNPKMARAS

SEQ ID NO: 160 - DNA, 48 bp, Artificial Sequence
  Description of Artificial Sequence Synthetic oligonucleotide
tgcaaagtgtgccagaactgcaagaatcctaaaatggctagagcaagt

SEQ ID NO: 161 - protein, 31 aa, Artificial Sequence
  Description of Artificial Sequence Synthetic polypeptide
DLFDEFDNSRLVSRSWFFFYLRNKWNLALGF

SEQ ID NO: 162 - DNA, 93 bp, Artificial Sequence
  Description of Artificial Sequence Synthetic oligonucleotide
gatctctttgatgagtttgacaactctagacttgtaagtcgcagttggttctttttctacttgcgcaacaagtggaatttggcactgggtttc

SEQ ID NO: 163 - protein, 16 aa, Artificial Sequence
  Description of Artificial Sequence Synthetic peptide
RSISLHKSDPSLLSPN

SEQ ID NO: 164 - DNA, 48 bp, Artificial Sequence
  Description of Artificial Sequence Synthetic oligonucleotide
cgatccatcagcctccacaagtcagatccatctctcctgtcaccaaat

SEQ ID NO: 165 - protein, 41 aa, Artificial Sequence
  Description of Artificial Sequence Synthetic polypeptide
FLQKHGNENTVLMLETLSEIRYGYVSCVCCPLLSPYGRCLN

SEQ ID NO: 166 - DNA, 123 bp, Artificial Sequence
  Description of Artificial Sequence Synthetic polynucleotide
ttcttacaaaaacatggaaatgaaaacacagttctgatgttagagaccttaagtgaaataaggtatgggtatgtgagctgtgtgtgctgccctttactaagtccttatggtagatgtctgaat

SEQ ID NO: 167 - protein, 44 aa, Artificial Sequence
  Description of Artificial Sequence Synthetic polypeptide
PPEILSNESLHGGIWPLRFWLPHWIISMERSQYGTQTRKTVYEN

SEQ ID NO: 168 - DNA, 132 bp, Artificial Sequence
  Description of Artificial Sequence Synthetic polynucleotide
cctcctgagattttgtctaatgaaagcttgcatggaggaatttggcccctaagattttggcttccccattggatcatcagtatggaacgcagtcagtatggaactcaaacaagaaagacagtgtatgaaaat

SEQ ID NO: 169 - protein, 21 aa, Artificial Sequence
  Description of Artificial Sequence Synthetic peptide
EAMDGREPHLLDKNDDRSISW

SEQ ID NO: 170 - DNA, 63 bp, Artificial Sequence
  Description of Artificial Sequence Synthetic oligonucleotide
gaagccatggatggccgggaacctcatctgttagacaaaaatgacgacagatccatttcctgg

SEQ ID NO: 171 - protein, 89 aa, Artificial Sequence
  Description of Artificial Sequence Synthetic polypeptide
SLLIGALRITWKREAGRKSFFKCGLRVHMWKGLQFIEITRRNWMLIFQSCSLFGTSQYRSLTKKCQLCFRFKRVSSKGSDITDKREVTH

SEQ ID NO: 172 - DNA, 267 bp, Artificial Sequence
  Description of Artificial Sequence Synthetic polynucleotide
tctctgctcattggggcgttgaggataacgtggaagcgggaagctgggagaaagtcattttttaaatgtggattgagagttcatatgtggaaagggctgcaattcattgaaataaccaggagaaattggatgctgatatttcagagctgcagtctgtttggaacctcacagtaccgcagtctaacaaagaagtgccagctttgttttagatttaaaagagtatcatctaaaggatctgacattacagacaaaagagaggtaactcac

SEQ ID NO: 173 - protein, 22 aa, Artificial Sequence
  Description of Artificial Sequence Synthetic peptide
TVPASQKLSCSLDPKHLRQFHL

SEQ ID NO: 174 - DNA, 66 bp, Artificial Sequence
  Description of Artificial Sequence Synthetic oligonucleotide
accgtccccgccagtcagaagttgagctgctcactggacccgaaacatctcagacagtttcacctc

SEQ ID NO: 175 - protein, 66 aa, Artificial Sequence
  Description of Artificial Sequence Synthetic polypeptide
MVMSFVNLALARLTQLQQESPARVWPSWEEGSSVRFSPLNCRLLPPNLECSFPTETFRESDSGWEL

SEQ ID NO: 176 - DNA, 198 bp, Artificial Sequence
  Description of Artificial Sequence Synthetic polynucleotide
atggtcatgagctttgtgaacctggctctggccaggttaactcaactgcagcaggagagtccagcccgagtgtggccatcctgggaagagggctcttcagtacgattttcccctctaaactgccgccttttgccccctaacttggagtgcagttttcccaccgagacttttagggaatccgacagcggctgggaactt

SEQ ID NO: 177 - protein, 39 aa, Artificial Sequence
  Description of Artificial Sequence Synthetic polypeptide
LEDLDTCMKPHAFQHAMMTFASTTFRLCQKSSKLSLLSS

SEQ ID NO: 178 - DNA, 117 bp, Artificial Sequence
  Description of Artificial Sequence Synthetic polynucleotide
ttggaagatctggatacctgtatgaaacctcatgcttttcaacatgccatgatgacatttgcatcaacgacgtttcgtttatgtcaaaaaagctccaaattatcactgctttccagt

SEQ ID NO: 179 - protein, 11 aa, Artificial Sequence
  Description of Artificial Sequence Synthetic peptide
FGTSHMMVRHF

SEQ ID NO: 180 - DNA, 33 bp, Artificial Sequence
  Description of Artificial Sequence Synthetic oligonucleotide
tttggcacatcacatatgatggtcagacatttt

SEQ ID NO: 181 - protein, 27 aa, Artificial Sequence
  Description of Artificial Sequence Synthetic peptide
GHFESIRAVLFHWIPVTTLLVSQVIYK

SEQ ID NO: 182 - DNA, 81 bp, Artificial Sequence
  Description of Artificial Sequence Synthetic oligonucleotide
ggacattttgaaagcatcagggccgtattatttcattggattcctgtaactacactgttagtcagccaagttatatataaa

SEQ ID NO: 183 - protein, 27 aa, Artificial Sequence
  Description of Artificial Sequence Synthetic peptide
MHTGTISQHCQLLDCSLGAILHIGYTK

SEQ ID NO: 184 - DNA, 81 bp, Artificial Sequence
  Description of Artificial Sequence Synthetic oligonucleotide
atgcatacaggaactatttctcagcattgtcagctcctggattgctccttgggcgctatactgcacattggctataccaag

SEQ ID NO: 185 - protein, 61 aa, Artificial Sequence
  Description of Artificial Sequence Synthetic polypeptide
EIEGSCHGGMITMDIKMGSYSCDYLKHSETLSAMVVSCGISFLFLFPRKKIAKLIFKEQVP

SEQ ID NO: 186 - DNA, 183 bp, Artificial Sequence
  Description of Artificial Sequence Synthetic polynucleotide
gaaattgagggttcttgccatggtggtatgatcactatggatatcaaaatgggaagttacagttgcgattacttaaaacattcagaaacattatctgctatggtggttagctgtggaatatcctttctctttctctttccaaggaaaaaaattgcaaaattgatttttaaggaacaagtacca

SEQ ID NO: 187 - protein, 12 aa, Artificial Sequence
  Description of Artificial Sequence Synthetic peptide
CEIMPLQSYILL

SEQ ID NO: 188 - DNA, 36 bp, Artificial Sequence
  Description of Artificial Sequence Synthetic oligonucleotide
tgtgaaatcatgcctctgcaaagttatatccttctc

SEQ ID NO: 189 - protein, 17 aa, Artificial Sequence
  Description of Artificial Sequence Synthetic peptide
VNRKSVKGFHVAHLPSA

SEQ ID NO: 190 - DNA, 51 bp, Artificial Sequence
  Description of Artificial Sequence Synthetic oligonucleotide
gttaatagaaaaagtgtcaaaggcttccacgtggctcatcttccttctgcc

SEQ ID NO: 191 - protein, 11 aa, Artificial Sequence
  Description of Artificial Sequence Synthetic peptide
FQDKYFMIATN

SEQ ID NO: 192 - DNA, 33 bp, Artificial Sequence
  Description of Artificial Sequence Synthetic oligonucleotide
ttccaggacaagtactttatgatagctacgaac

SEQ ID NO: 193 - protein, 16 aa, Artificial Sequence
  Description of Artificial Sequence Synthetic peptide
VAQVEIRGLEPPVHDQ

SEQ ID NO: 194 - DNA, 48 bp, Artificial Sequence
  Description of Artificial Sequence Synthetic oligonucleotide
gtagcgcaggtggaaataagaggactagaaccaccagtgcatgatcaa

SEQ ID NO: 195 - protein, 43 aa, Artificial Sequence
  Description of Artificial Sequence Synthetic polypeptide
GGGGGSGELTTQIPCSWRTKGHIHDKKTEPFRLLAWSWCLNVS

SEQ ID NO: 196 - DNA, 129 bp, Artificial Sequence
  Description of Artificial Sequence Synthetic polynucleotide
ggcgggggaggcggcagcggagagctaacaacacagatcccatgtagttggagaaccaaaggccacatacatgacaaaaagactgaaccgttcaggttacttgcatggagttggtgcttaaatgtgagt

SEQ ID NO: 197 - protein, 18 aa, Artificial Sequence
  Description of Artificial Sequence Synthetic peptide
VAPSQSRQEECWRHKMSW

SEQ ID NO: 198 - DNA, 54 bp, Artificial Sequence
  Description of Artificial Sequence Synthetic oligonucleotide
gtagcaccttcacagtccaggcaagaggaatgctggcgtcataaaatgagttgg

SEQ ID NO: 199 - protein, 13 aa, Artificial Sequence
  Description of Artificial Sequence Synthetic peptide
IALQYCCTCSISQ

SEQ ID NO: 200 - DNA, 39 bp, Artificial Sequence
  Description of Artificial Sequence Synthetic oligonucleotide
atagcccttcagtactgttgtacgtgttccatttctcaa

SEQ ID NO: 201 - protein, 15 aa, Artificial Sequence
  Description of Artificial Sequence Synthetic peptide
SAVDDMIAAFPFKRA

SEQ ID NO: 202 - DNA, 45 bp, Artificial Sequence
  Description of Artificial Sequence Synthetic oligonucleotide
agtgctgtagatgacatgatagcagcatttcccttcaaaagagct

SEQ ID NO: 203 - protein, 23 aa, Artificial Sequence
  Description of Artificial Sequence Synthetic peptide
MWTRKARGTTICDRRRQICRWRE

SEQ ID NO: 204 - DNA, 69 bp, Artificial Sequence
  Description of Artificial Sequence Synthetic oligonucleotide
atgtggacaaggaaagcaagaggaactaccatttgtgatagacgaagacagatttgcaggtggagggag

SEQ ID NO: 205 - protein, 12 aa, Artificial Sequence
  Description of Artificial Sequence Synthetic peptide
INYRLGILDNVS

SEQ ID NO: 206 - DNA, 36 bp, Artificial Sequence
  Description of Artificial Sequence Synthetic oligonucleotide
attaactaccgtctgggaatactagataatgtttca

SEQ ID NO: 207 - protein, 25 aa, Artificial Sequence
  Description of Artificial Sequence Synthetic peptide
TSTSPKFPVFIFRLVCWVRTELVLR

SEQ ID NO: 208 - DNA, 75 bp, Artificial Sequence
  Description of Artificial Sequence Synthetic oligonucleotide
actagcaccagccccaaatttcctgtttttatctttagactcgtgtgttgggtaaggacagagctggtactgcgt

SEQ ID NO: 209 - protein, 20 aa, Artificial Sequence
  Description of Artificial Sequence Synthetic peptide
MAIPGRQNIVLRSHRKRVGI

SEQ ID NO: 210 - DNA, 60 bp, Artificial Sequence
  Description of Artificial Sequence Synthetic oligonucleotide
atggcgattccgggcaggcagaatattgttctgagaagccacagaaaaagggtaggcata

SEQ ID NO: 211 - protein, 23 aa, Artificial Sequence
  Description of Artificial Sequence Synthetic peptide
EAEFDKTTRTFLQIFNRKRGKIQ

SEQ ID NO: 212 - DNA, 69 bp, Artificial Sequence
  Description of Artificial Sequence Synthetic oligonucleotide
gaggctgaatttgataagaccactagaacatttctacaaattttcaatcgcaaaagaggaaaaatacaa

SEQ ID NO: 213 - protein, 12 aa, Artificial Sequence
  Description of Artificial Sequence Synthetic peptide
KLLYEVSQKMVL

SEQ ID NO: 214 - DNA, 36 bp, Artificial Sequence
  Description of Artificial Sequence Synthetic oligonucleotide
aaactgctgtatgaagtttcccagaaaatggtctta

SEQ ID NO: 215 - protein, 20 aa, Artificial Sequence
  Description of Artificial Sequence Synthetic peptide
WAFIHSLPALLGIFSYPPSR

SEQ ID NO: 216 - DNA, 60 bp, Artificial Sequence
  Description of Artificial Sequence Synthetic oligonucleotide
tgggccttcatccattctttgcctgcactactgggtattttctcataccccccaagtcga

SEQ ID NO: 217 - protein, 11 aa, Artificial Sequence
  Description of Artificial Sequence Synthetic peptide
SSLQEHRKLLS

SEQ ID NO: 218 - DNA, 33 bp, Artificial Sequence
  Description of Artificial Sequence Synthetic oligonucleotide
tcaagcctacaggaacatagaaagcttctctcc

SEQ ID NO: 219 - protein, 58 aa, Artificial Sequence
  Description of Artificial Sequence Synthetic polypeptide
RFSRRYPCSARTSLGREESEPPGEAAMWRWRLWRGRASGHPSGEGRPPETNQGHSTGR

SEQ ID NO: 220 - DNA, 174 bp, Artificial Sequence
  Description of Artificial Sequence Synthetic polynucleotide
cgattttctcggcggtatccatgcagtgctaggaccagccttgggagagaggaaagtgagccaccaggagaagctgccatgtggaggtggaggctctggaggggtcgggcatcaggacacccttcaggagaggggagacctcctgagacaaaccaagggcattccactgggaga

SEQ ID NO: 221 - protein, 32 aa, Artificial Sequence
  Description of Artificial Sequence Synthetic polypeptide
ERSHSKKKDGLVRNDGPGFPPMFSTSENTNLD

SEQ ID NO: 222 - DNA, 96 bp, Artificial Sequence
  Description of Artificial Sequence Synthetic oligonucleotide
gaaagaagccactccaaaaagaaggatggtcttgtgagaaatgacggacctggattcccacctatgttttccacatcagaaaataccaacttggat

SEQ ID NO: 223 - protein, 27 aa, Artificial Sequence
  Description of Artificial Sequence Synthetic peptide
AGDVLFIPGISLKVHKVQHSKVINICN

SEQ ID NO: 224 - DNA, 81 bp, Artificial Sequence
  Description of Artificial Sequence Synthetic oligonucleotide
gctggtgatgtattattcattcctggcattagtttgaaagtccataaagttcaacacagcaaggtaattaacatctgtaat

SEQ ID NO: 225 - protein, 15 aa, Artificial Sequence
  Description of Artificial Sequence Synthetic peptide
IENEGGQVRSRPAPS

SEQ ID NO: 226 - DNA, 45 bp, Artificial Sequence
  Description of Artificial Sequence Synthetic oligonucleotide
atcgagaacgaaggcggccaggtgaggtcccgccccgctccctcc

SEQ ID NO: 227 - protein, 57 aa, Artificial Sequence
  Description of Artificial Sequence Synthetic polypeptide
LISVVQNGKESPSLREEQKGRFSLMVVWKSGLNNCVCVCACTNTFTLSLFSSFYLNV

SEQ ID NO: 228 - DNA, 171 bp, Artificial Sequence
  Description of Artificial Sequence Synthetic polynucleotide
ctcatcagcgtggtccagaatggtaaggaaagcccttcactcagggaagaacagaaggggagattttctttgatggttgtttggaagtcaggcttaaacaattgtgtctgtgtgtgcgcatgcacaaacacttttaccttatctttattttcttctttttatttgaatgta

SEQ ID NO: 229 - protein, 26 aa, Artificial Sequence
  Description of Artificial Sequence Synthetic peptide
EHQRNSSKVFYGVIDCVIKETITWHD

SEQ ID NO: 230 - DNA, 78 bp, Artificial Sequence
  Description of Artificial Sequence Synthetic oligonucleotide
gaacatcaaagaaatagcagtaaggtattttatggtgttattgactgtgtcataaaggaaactattacttggcacgat

SEQ ID NO: 231 - protein, 24 aa, Artificial Sequence
  Description of Artificial Sequence Synthetic peptide
DSLPKTEMVSGSYRMGLCEHCLNQ

SEQ ID NO: 232 - DNA, 72 bp, Artificial Sequence
  Description of Artificial Sequence Synthetic oligonucleotide
gactcgcttcccaaaactgaaatggtgagtgggtcatacagaatgggtctgtgtgagcactgtcttaatcaa

SEQ ID NO: 233 - protein, 54 aa, Artificial Sequence
  Description of Artificial Sequence Synthetic polypeptide
RPRKDTWKVRDPALRYTPLLIQSLDSRLAESCAVFIWYCIFTLGDTCRSRPVEN

SEQ ID NO: 234 - DNA, 162 bp, Artificial Sequence
  Description of Artificial Sequence Synthetic polynucleotide
cgcccgcggaaagacacctggaaggttagagatccagcattgcgctacacccctttgttaattcagtcactggacagccgcctagccgagagctgtgcggtttttatatggtattgtatctttactttaggcgatacatgcagaagtcgtccggtagaaaac

SEQ ID NO: 235 - protein, 16 aa, Artificial Sequence
  Description of Artificial Sequence Synthetic peptide
PVGSVSFQVGCVQLFP

SEQ ID NO: 236 - DNA, 48 bp, Artificial Sequence
  Description of Artificial Sequence Synthetic oligonucleotide
cccgtgggcagtgtcagcttccaggtagggtgtgtgcagcttttccct

SEQ ID NO: 237 - protein, 18 aa, Artificial Sequence
  Description of Artificial Sequence Synthetic peptide
QRIQAQEGKCPVNLSQPL

SEQ ID NO: 238 - DNA, 54 bp, Artificial Sequence
  Description of Artificial Sequence Synthetic oligonucleotide
cagaggattcaggcacaggaaggtaagtgtcctgtaaatctctcccagcccctt

SEQ ID NO: 239 - protein, 14 aa, Artificial Sequence
  Description of Artificial Sequence Synthetic peptide
NLKPDRKGKESMAK

SEQ ID NO: 240 - DNA, 42 bp, Artificial Sequence
  Description of Artificial Sequence Synthetic oligonucleotide
aacctcaagccagacagaaaaggtaaggagagcatggcaaag

SEQ ID NO: 241 - protein, 48 aa, Artificial Sequence
  Description of Artificial Sequence Synthetic polypeptide
DLNQDGYNGNLNQSVEVIYVSSPLHKGDHACFKVFSMVCVGAVIITDF

SEQ ID NO: 242 - DNA, 144 bp, Artificial Sequence
  Description of Artificial Sequence Synthetic polynucleotide
gacctgaaccaagatggatacaatggtaatttaaaccaaagcgtggaagtcatttatgtgtcgtcaccactgcataaaggagaccatgcctgttttaaagtattcagtatggtgtgtgtgggtgcagttattattactgatttt

SEQ ID NO: 243 - protein, 15 aa, Artificial Sequence
  Description of Artificial Sequence Synthetic peptide
APHLLRLFGNMSCRK

SEQ ID NO: 244 - DNA, 45 bp, Artificial Sequence
  Description of Artificial Sequence Synthetic oligonucleotide
gcgccacatctcctgagattatttggtaatatgtcatgtagaaaa

SEQ ID NO: 245 - protein, 23 aa, Artificial Sequence
  Description of Artificial Sequence Synthetic peptide
SLKKVVKEVGVESWRGRGAWRGA

SEQ ID NO: 246 - DNA, 69 bp, Artificial Sequence
  Description of Artificial Sequence Synthetic oligonucleotide
tccctgaagaaagttgtgaaggaggtgggtgttgagagttggagagggagaggtgcgtggaggggagcc

SEQ ID NO: 247 - protein, 13 aa, Artificial Sequence
  Description of Artificial Sequence Synthetic peptide
PVILFKGKVRPIP

SEQ ID NO: 248 - DNA, 39 bp, Artificial Sequence
  Description of Artificial Sequence Synthetic oligonucleotide
ccagtaatcctgttcaagggcaaggtaaggcccatacca

SEQ ID NO: 249 - protein, 18 aa, Artificial Sequence
  Description of Artificial Sequence Synthetic peptide
QILEQPKQVRLKGWDFKC

SEQ ID NO: 250 - DNA, 54 bp, Artificial Sequence
  Description of Artificial Sequence Synthetic oligonucleotide
caaatattagagcaacctaaacaggtaagattaaaggggtgggactttaaatgt

SEQ ID NO: 251 - protein, 26 aa, Artificial Sequence
  Description of Artificial Sequence Synthetic peptide
IATGAQVVTQDTNYFLFCFEKFVPVH

SEQ ID NO: 252 - DNA, 78 bp, Artificial Sequence
  Description of Artificial Sequence Synthetic oligonucleotide
atagcaactggagctcaggtagtaacacaggatactaattatttcttattttgctttgaaaaatttgttccagtccac

SEQ ID NO: 253 - protein, 16 aa, Artificial Sequence
  Description of Artificial Sequence Synthetic peptide
LLYEVSQVSAGSDTSN

SEQ ID NO: 254 - DNA, 48 bp, Artificial Sequence
  Description of Artificial Sequence Synthetic oligonucleotide
ctgctgtatgaagtttcccaggtgagtgcagggtctgataccagcaac

SEQ ID NO: 255 - protein, 34 aa, Artificial Sequence
  Description of Artificial Sequence Synthetic polypeptide
WKLKATKVNGTPDAYGESMRALWGGSAFQLGGFQ

SEQ ID NO: 256 - DNA, 102 bp, Artificial Sequence
  Description of Artificial Sequence Synthetic polynucleotide
tggaagctaaaggccactaaggtaaacggcactcctgatgcttatggagagagtatgagggccttgtggggagggtctgcattccaacttgggggtttccag

SEQ ID NO: 257 - protein, 24 aa, Artificial Sequence
  Description of Artificial Sequence Synthetic peptide
SPAIYKLKVNIQNIKGENLRHNHF

SEQ ID NO: 258 - DNA, 72 bp, Artificial Sequence
  Description of Artificial Sequence Synthetic oligonucleotide
agtcctgcaatttataaactaaaggtaaacatacaaaacataaagggagaaaacttaagacataaccatttc

SEQ ID NO: 259 - protein, 26 aa, Artificial Sequence
  Description of Artificial Sequence Synthetic peptide
DMEVFLKEASTNQHNTLIHLTLTFST

SEQ ID NO: 260 - DNA, 78 bp, Artificial Sequence
  Description of Artificial Sequence Synthetic oligonucleotide
gatatggaggttttcctcaaagaggcaagtactaaccaacacaacactttgattcacttgacacttactttcagcact

SEQ ID NO: 261 - protein, 16 aa, Artificial Sequence
  Description of Artificial Sequence Synthetic peptide
LQANQLQGVSKGNPVC

SEQ ID NO: 262 - DNA, 48 bp, Artificial Sequence
  Description of Artificial Sequence Synthetic oligonucleotide
cttcaagcgaatcagctacaaggggtaagtaaaggaaacccagtttgt

SEQ ID NO: 263 - protein, 35 aa, Artificial Sequence
  Description of Artificial Sequence Synthetic polypeptide
LTDDQVSGKLVSNSFTPFVSFIFPFFPFLLSLIEE

SEQ ID NO: 264 - DNA, 105 bp, Artificial Sequence
  Description of Artificial Sequence Synthetic polynucleotide
ttaacagatgatcaggtttcaggtaagttggtttccaactcctttacacccttcgtttccttcatctttccgttcttccctttccttctttctttaattgaagaa

SEQ ID NO: 265 - protein, 23 aa, Artificial Sequence
  Description of Artificial Sequence Synthetic peptide
QRDRDSIKVGLKIYRLRNGLKGL

SEQ ID NO: 266 - DNA, 69 bp, Artificial Sequence
  Description of Artificial Sequence Synthetic oligonucleotide
caaagagacagagactccatcaaggtaggactcaaaatttacagactaaggaatggcttaaaagggctg

SEQ ID NO: 267 - protein, 23 aa, Artificial Sequence
  Description of Artificial Sequence Synthetic peptide
KRYGCVFQVSIRHLSFPVTDILP

SEQ ID NO: 268 - DNA, 69 bp, Artificial Sequence
  Description of Artificial Sequence Synthetic oligonucleotide
aagcgctatggctgcgtttttcaggtcagtatccgacatttgtccttcccagtcactgacattctgcca

SEQ ID NO: 269 - protein, 16 aa, Artificial Sequence
  Description of Artificial Sequence Synthetic peptide
EGTPFEDGKSYSLCFL

SEQ ID NO: 270 - DNA, 48 bp, Artificial Sequence
  Description of Artificial Sequence Synthetic oligonucleotide
gaagggacaccttttgaagatggtaagtcatactcattatgttttcta

SEQ ID NO: 271 - protein, 31 aa, Artificial Sequence
  Description of Artificial Sequence Synthetic polypeptide
SMTQALCRYFFMTLLMVIVDLGKRKYFVFCE

SEQ ID NO: 272 - DNA, 93 bp, Artificial Sequence
  Description of Artificial Sequence Synthetic oligonucleotide
agtatgacccaagccctttgcaggtacttcttcatgacactattaatggtaattgtagatttggggaagaggaagtattttgtattttgtgag

SEQ ID NO: 273 - protein, 29 aa, Artificial Sequence
  Description of Artificial Sequence Synthetic peptide
LLDGMIFQVSRSVSDCNSGAHIRRQSSEL

SEQ ID NO: 274 - DNA, 87 bp, Artificial Sequence
  Description of Artificial Sequence Synthetic oligonucleotide
ttactggatggaatgatattccaggtaagtaggtctgtatcagactgtaacagtggggctcatattagacgacaaagctcagaatta

SEQ ID NO: 275 - protein, 17 aa, Artificial Sequence
  Description of Artificial Sequence Synthetic peptide
EIPRKTIFGKNQVDMRP

SEQ ID NO: 276 - DNA, 51 bp, Artificial Sequence
  Description of Artificial Sequence Synthetic oligonucleotide
gaaataccaaggaagactatttttggtaagaaccaggtagatatgaggcca

SEQ ID NO: 277 - protein, 29 aa, Artificial Sequence
  Description of Artificial Sequence Synthetic peptide
SQKQPAEKCLLRNNQLRRLQVTRKILFQI

SEQ ID NO: 278 - DNA, 87 bp, Artificial Sequence
  Description of Artificial Sequence Synthetic oligonucleotide
tctcagaaacaaccagctgagaagtgtcttctcagaaacaaccagctgagaaggctacaagtgacgagaaagattctgtttcaaata

SEQ ID NO: 279 - protein, 16 aa, Artificial Sequence
  Description of Artificial Sequence Synthetic peptide
SRKKPALKCLLRNNQH

SEQ ID NO: 280 - DNA, 48 bp, Artificial Sequence
  Description of Artificial Sequence Synthetic oligonucleotide
tctcggaaaaaaccagccttgaagtgtcttctccgaaacaaccagcat

SEQ ID NO: 281 - protein, 16 aa, Artificial Sequence
  Description of Artificial Sequence Synthetic peptide
SQKQPALKCLLRNNRP

SEQ ID NO: 282 - DNA, 48 bp, Artificial Sequence
  Description of Artificial Sequence Synthetic oligonucleotide
tctcagaaacaaccagccttgaagtgtcttctcagaaacaaccggcct

SEQ ID NO: 283 - protein, 16 aa, Artificial Sequence
  Description of Artificial Sequence Synthetic peptide
SEQPPGLKCLLGKKQP

SEQ ID NO: 284 - DNA, 48 bp, Artificial Sequence
  Description of Artificial Sequence Synthetic oligonucleotide
tctgagcaaccaccaggcttgaagtgtcttctcggaaaaaagcagcct

SEQ ID NO: 285 - protein, 16 aa, Artificial Sequence
  Description of Artificial Sequence Synthetic peptide
VYYLYKNVAYKGSDID

SEQ ID NO: 286 - DNA, 48 bp, Artificial Sequence
  Description of Artificial Sequence Synthetic oligonucleotide
gtgtactacctctacaagaacgtggcctataagggctctgatatagat

SEQ ID NO: 287 - protein, 16 aa, Artificial Sequence
  Description of Artificial Sequence Synthetic peptide
QLSVVTRDDYNSSELK

SEQ ID NO: 288 - DNA, 48 bp, Artificial Sequence
  Description of Artificial Sequence Synthetic oligonucleotide
cagctgagcgtggtgacaagggatgattacaacagcagtgaattgaaa

SEQ ID NO: 289 - protein, 31 aa, Artificial Sequence
  Description of Artificial Sequence Synthetic polypeptide
LEDKELGRISVCPWTEGLEKMEKTLLCSSSG

SEQ ID NO: 290 - DNA, 93 bp, Artificial Sequence
  Description of Artificial Sequence Synthetic oligonucleotide
ctggaagataaagaactgggaaggatatctgtatgcccttggacagaaggtttggaaaagatggaaaaaacgttactttgttctagttcaggt

SEQ ID NO: 291 - protein, 16 aa, Artificial Sequence
  Description of Artificial Sequence Synthetic peptide
ENQRLSKKDLVSEKLK

SEQ ID NO: 292 - DNA, 48 bp, Artificial Sequence
  Description of Artificial Sequence Synthetic oligonucleotide
gaaaatcaaaggctcagtaaaaaggatttggtgagtgaaaagttgaag

SEQ ID NO: 293 - protein, 99 aa, Artificial Sequence
  Description of Artificial Sequence Synthetic polypeptide
MSATTGSQDNPEGKRDPSRKFQEQKALPKRPPLQKNLRNGVGQHYPATLTSRTRNSIKMRHSRTTRARRPPPSPVLPWAASNSQARPETSSLSEKSPEV

SEQ ID NO: 294 - DNA, 297 bp, Artificial Sequence
  Description of Artificial Sequence Synthetic polynucleotide
atgtcggcgactacaggttcccaagacaacccagaagggaaaagggacccgtcaaggaagttccaggaacaaaaggctctccctaaaagaccaccgcttcaaaaaaacctgaggaatggagtgggccaacactatccagccactctgaccagccgaacgaggaactcaatcaaaatgcgccatagcaggaccacaagggcaaggagaccaccgccttctccagtgcttccttgggcagccagtaattcccaggcaaggccagagacttcaagtctatctgaaaagtctccagaagtc

SEQ ID NO: 295 - protein, 13 aa, Artificial Sequence
  Description of Artificial Sequence Synthetic peptide
QDLATEDTGSVTK

SEQ ID NO: 296 - DNA, 39 bp, Artificial Sequence
  Description of Artificial Sequence Synthetic oligonucleotide
caggacctcgccacagaggataccggaagtgtgacaaaa

SEQ ID NO: 297 - protein, 31 aa, Artificial Sequence
  Description of Artificial Sequence Synthetic polypeptide
DPTTKRSRLTPKLHFLVERNPRWSQEQRKYL

SEQ ID NO: 298 - DNA, 93 bp, Artificial Sequence
  Description of Artificial Sequence Synthetic oligonucleotide
gatcccactacgaaacgctccagattgaccccaaagttgcatttcctcgtcgagcgcaacccaagatggtcacaagaacaaagaaaatatttg

SEQ ID NO: 299 - protein, 12 aa, Artificial Sequence
  Description of Artificial Sequence Synthetic peptide
ILAQKKKDELSS

SEQ ID NO: 300 - DNA, 36 bp, Artificial Sequence
  Description of Artificial Sequence Synthetic oligonucleotide
atattggctcagaagaagaaggatgagctgagcagc

SEQ ID NO: 301 - protein, 16 aa, Artificial Sequence
  Description of Artificial Sequence Synthetic peptide
AKFWSDISINRAPSFG

SEQ ID NO: 302 - DNA, 48 bp, Artificial Sequence
  Description of Artificial Sequence Synthetic oligonucleotide
gctaagttttggagtgatatttcaattaaccgagccccaagctttgga

SEQ ID NO: 303 - protein, 27 aa, Artificial Sequence
  Description of Artificial Sequence Synthetic peptide
LDPPHDEKALACPQWTLLWIFWTAKAL

SEQ ID NO: 304 - DNA, 81 bp, Artificial Sequence
  Description of Artificial Sequence Synthetic oligonucleotide
ctggatccacctcatgatgaaaaggctctggcatgccctcaatggacattattgtggatattttggacagcgaaggctctt

SEQ ID NO: 305 - protein, 14 aa, Artificial Sequence
  Description of Artificial Sequence Synthetic peptide
KKEDVNFQVKQLIH

SEQ ID NO: 306 - DNA, 42 bp, Artificial Sequence
  Description of Artificial Sequence Synthetic oligonucleotide
aagaaagaagatgttaactttcaggtaaaacagttaattcac

SEQ ID NO: 307 - protein, 18 aa, Artificial Sequence
  Description of Artificial Sequence Synthetic peptide
GNVLVRYVSLYVYYILLN

SEQ ID NO: 308 - DNA, 54 bp, Artificial Sequence
  Description of Artificial Sequence Synthetic oligonucleotide
ggaaatgtattggtaagatatgtaagtttatatgtttactatattctactgaac

SEQ ID NO: 309 - protein, 16 aa, Artificial Sequence
  Description of Artificial Sequence Synthetic peptide
QRSFIKAASKSFLSQF

SEQ ID NO: 310 - DNA, 48 bp, Artificial Sequence
  Description of Artificial Sequence Synthetic oligonucleotide
caaagaagtttcatcaaagcagcaagtaagtcttttttgtctcagttt

SEQ ID NO: 311 - protein, 23 aa, Artificial Sequence
  Description of Artificial Sequence Synthetic peptide
FSGRSRSRGKTRRDGFLRATRNN

SEQ ID NO: 312 - DNA, 69 bp, Artificial Sequence
  Description of Artificial Sequence Synthetic oligonucleotide
ttctcgggtcgttcaagaagccgaggtaagacacgccgcgacgggtttctgcgggctaccaggaacaat

SEQ ID NO: 313 - protein, 17 aa, Artificial Sequence
  Description of Artificial Sequence Synthetic peptide
RESKRHQGKRWTLFPHR

SEQ ID NO: 314 - DNA, 51 bp, Artificial Sequence
  Description of Artificial Sequence Synthetic oligonucleotide
cgagaaagtaagaggcaccagggtaaacgatggactctctttcctcatcgt

SEQ ID NO: 315 - protein, 52 aa, Artificial Sequence
  Description of Artificial Sequence Synthetic polypeptide
HLAIPAKISECRSPAQSRLPRQRDPGAGRSPGFSSCPQPGRSWTKWRQQCTR

SEQ ID NO: 316 - DNA, 156 bp, Artificial Sequence
  Description of Artificial Sequence Synthetic polynucleotide
cacctggccatccccgccaagatcagtgagtgccggagcccagcccagtcccgactaccccgccagcgagaccccggggcaggccggtcacctggcttctcctcctgcccgcagcccgggagaagctggaccaagtggcgacagcagtgtaccaga

SEQ ID NO: 317 - protein, 32 aa, Artificial Sequence
  Description of Artificial Sequence Synthetic polypeptide
LDLANPQGKGPKGELRDFAPRTEEAPVCPALP

SEQ ID NO: 318 - DNA, 96 bp, Artificial Sequence
  Description of Artificial Sequence Synthetic oligonucleotide
ttagacctggccaatcctcaaggtaagggccctaagggagaactgagggacttcgcaccaaggacagaagaagccccggtctgccctgcgctgcca

SEQ ID NO: 319 - protein, 27 aa, Artificial Sequence
  Description of Artificial Sequence Synthetic peptide
RDLRSIVEVRRADTRGQWVPRPAPWPP

SEQ ID NO: 320 - DNA, 81 bp, Artificial Sequence
  Description of Artificial Sequence Synthetic oligonucleotide
agggacctccgcagcatcgtggaggtaaggcgggcagacaccagagggcagtgggtacccaggccagccccttggccccca

SEQ ID NO: 321 - protein, 60 aa, Artificial Sequence
  Description of Artificial Sequence Synthetic polypeptide
LLTEMDNKVRQGHGGALHPTALPPGSGQHHLLLGQPPTCFLRGPLAPLSPPQLDRLELDP

SEQ ID NO: 322 - DNA, 180 bp, Artificial Sequence
  Description of Artificial Sequence Synthetic polynucleotide
ctgctcacagagatggacaacaaggtgaggcaggggcatggcggggctctccatcccacagccttgcccccaggatcgggacagcaccaccttctcctgggtcagccccctacctgcttccttagaggtcccctggcccctctttccccgccacagctggaccgcctggagctggaccca

SEQ ID NO: 323 - protein, 15 aa, Artificial Sequence
  Description of Artificial Sequence Synthetic peptide
MLRRCPCVTNVVLAL

SEQ ID NO: 324 - DNA, 45 bp, Artificial Sequence
  Description of Artificial Sequence Synthetic oligonucleotide
atgctcagaaggtgcccatgtgtgacaaatgtggtcctggcattg

SEQ ID NO: 325 - protein, 15 aa, Artificial Sequence
  Description of Artificial Sequence Synthetic peptide
AGKERSTQVRVGPTL

SEQ ID NO: 326 - DNA, 45 bp, Artificial Sequence
  Description of Artificial Sequence Synthetic oligonucleotide
gcaggaaaggagcgtagcacccaggtaagggtgggtcccactctg

SEQ ID NO: 327 - protein, 74 aa, Artificial Sequence
  Description of Artificial Sequence Synthetic polypeptide
AGRSLCLTVSSTPGSWGASGFRRLGRLTSFNFGSESLWLFYREAAFQGLRSRHPFSNPLHLDPTSSPWRKNISV

SEQ ID NO: 328 - DNA, 222 bp, Artificial Sequence
  Description of Artificial Sequence Synthetic polynucleotide
gcggggcgaagtctgtgtctcacggtcagttcaactccaggctcctggggagcgtctgggttccggcgactaggacgcctaacttcctttaacttcgggtctgaaagtctgtggctcttttacagagaagcagcgttccagggactccgttcccgccacccgttcagcaaccctctacacctggacccgacctcctcgccttggaggaagaatataagcgtt

SEQ ID NO: 329 - protein, 39 aa, Artificial Sequence
  Description of Artificial Sequence Synthetic polypeptide
VEDEEKKEAGTHFIHLTGTTVSAGVPEEMPATTLRREVF

SEQ ID NO: 330 - DNA, 117 bp, Artificial Sequence
  Description of Artificial Sequence Synthetic polynucleotide
gttgaagatgaggagaagaaagaggcagggactcatttcatccacctgactggaaccactgtctcagctggagtccctgaggagatgccagccacaactctccgaagagaagtattc

SEQ ID NO: 331 - protein, 57 aa, Artificial Sequence
  Description of Artificial Sequence Synthetic polypeptide
TAILFTRQQSKALPSLGITNRHGFWAGQGNTVMHGRDSDGKQVKGTVVILAQGAFHQ

SEQ ID NO: 332 - DNA, 171 bp, Artificial Sequence
  Description of Artificial Sequence Synthetic polynucleotide
actgccatcctcttcaccaggcagcaaagcaaggccctcccatccttggggattacaaacagacatggattttgggctggacaaggaaatacagtgatgcatggcagagattcggatgggaaacaagttaaaggtacagtagtaatactagcgcagggtgcttttcaccag

SEQ ID NO: 333 - protein, 31 aa, Artificial Sequence
  Description of Artificial Sequence Synthetic polypeptide
NASLHEVKDFFVPFPLPLLHLTSLKRNKLFN

SEQ ID NO: 334 - DNA, 93 bp, Artificial Sequence
  Description of Artificial Sequence Synthetic oligonucleotide
aatgcttcactacatgaagtcaaagatttctttgttcctttcccactcccactacttcatttgactagccttaaaagaaataaattatttaat

SEQ ID NO: 335 - protein, 12 aa, Artificial Sequence
  Description of Artificial Sequence Synthetic peptide
GTTRLLSGEDFS

SEQ ID NO: 336 - DNA, 36 bp, Artificial Sequence
  Description of Artificial Sequence Synthetic oligonucleotide
ggtactacccgtcttctatctggggaggacttttcc

SEQ ID NO: 337 - protein, 27 aa, Artificial Sequence
  Description of Artificial Sequence Synthetic peptide
SEMVAHERYVFEATGNDSFRTLCLLMK

SEQ ID NO: 338 - DNA, 81 bp, Artificial Sequence
  Description of Artificial Sequence Synthetic oligonucleotide
tctgaaatggtggcacatgaaaggtatgtttttgaagctaccggaaatgattcttttagaactctttgtttattgatgaaa

SEQ ID NO: 339 - protein, 33 aa, Artificial Sequence
  Description of Artificial Sequence Synthetic polypeptide
IIMALAILDLFAVLKIEAHKQLLLKSFCYGAGK

SEQ ID NO: 340 - DNA, 99 bp, Artificial Sequence
  Description of Artificial Sequence Synthetic oligonucleotide
ataattatggcattagccatcctggacctctttgcagtcttaaaaattgaagcccacaaacagctcttacttaagagtttctgctatggtgctggaaaa

SEQ ID NO: 341 - protein, 11 aa, Artificial Sequence
  Description of Artificial Sequence Synthetic peptide
QIRVPSYSDEG

SEQ ID NO: 342 - DNA, 33 bp, Artificial Sequence
  Description of Artificial Sequence Synthetic oligonucleotide
caaattagggtaccatcatattcagatgaaggc

SEQ ID NO: 343 - protein, 84 aa, Artificial Sequence
  Description of Artificial Sequence Synthetic polypeptide
RQREVVDLVKKDLVMRAVSLGTQRGDQNGRPSWSGHAIWARIMECAYKGQQECLVETGALGPMAFRVHLGSQVGMDSKEKRGNV

SEQ ID NO: 344 - DNA, 252 bp, Artificial Sequence
  Description of Artificial Sequence Synthetic polynucleotide
cggcagagggaggtggtggacctggtgaagaaggacctggttatgcgtgcagtgagtcttggaactcagaggggagaccaaaatgggaggccatcgtggagcggacatgcgatatgggcccgtataatggaatgtgcttacaagggccagcaggagtgcctggtcgagacgggagccctggggccaatggcattccgggtacacctgggatcccaggtcgggatggattcaaaggagaaaagggggaatgtc

SEQ ID NO: 345 - protein, 30 aa, Artificial Sequence
  Description of Artificial Sequence Synthetic polypeptide
VHLDFSSAEMGFPHAAQANVELLGSSDLLT

SEQ ID NO: 346 - DNA, 90 bp, Artificial Sequence
  Description of Artificial Sequence Synthetic oligonucleotide
gtgcacttggatttttcttcagcagagatgggatttcctcatgctgcccaggctaatgttgaactcctgggctcaagtgatctgctcacc

SEQ ID NO: 347 - protein, 34 aa, Artificial Sequence
  Description of Artificial Sequence Synthetic polypeptide
GEKILFIRGLHGCDYISTAKAKQTFKMLHNKHVM

SEQ ID NO: 348 - DNA, 102 bp, Artificial Sequence
  Description of Artificial Sequence Synthetic polynucleotide
ggagaaaagatcctcttcattaggggtcttcatggttgtgactatatctccacagccaaagcaaagcagacatttaagatgctgcacaacaaacatgtcatg

SEQ ID NO: 349 - protein, 33 aa, Artificial Sequence
  Description of Artificial Sequence Synthetic polypeptide
GFFETEMAVPFFSRKIRKVRRAIMKWTFCCYFY

SEQ ID NO: 350 - DNA, 99 bp, Artificial Sequence
  Description of Artificial Sequence Synthetic oligonucleotide
ggcttctttgagactgaaatggcagtccccttcttttccagaaaaattagaaaagtcagaagagcaataatgaaatggacattttgctgttatttctat

SEQ ID NO: 351 - protein, 32 aa, Artificial Sequence
  Description of Artificial Sequence Synthetic polypeptide
DSQVETTVWGVGNRQEWREILYGSTQNFASLL

SEQ ID NO: 352 - DNA, 96 bp, Artificial Sequence
  Description of Artificial Sequence Synthetic oligonucleotide
gacagtcaagtggaaacaactgtctggggagttggaaataggcaagaatggagagaaattttgtatggcagtacacagaattttgcaagtttattg

SEQ ID NO: 353 - protein, 18 aa, Artificial Sequence
  Description of Artificial Sequence Synthetic peptide
WTRPEKQELNSSCCSLIL

SEQ ID NO: 354 - DNA, 54 bp, Artificial Sequence
  Description of Artificial Sequence Synthetic oligonucleotide
tggactcgaccagagaagcaagagctcaactcatcttgttgctcacttatttta

SEQ ID NO: 355 - protein, 15 aa, Artificial Sequence
  Description of Artificial Sequence Synthetic peptide
LGRLEEAKSFETEEN

SEQ ID NO: 356 - DNA, 45 bp, Artificial Sequence
  Description of Artificial Sequence Synthetic oligonucleotide
ctgggtcgcttggaagaagccaagtcctttgaaactgaggaaaac

SEQ ID NO: 357 - protein, 16 aa, Artificial Sequence
  Description of Artificial Sequence Synthetic peptide
LGRLEEAKVPLKMLLF

SEQ ID NO: 358 - DNA, 48 bp, Artificial Sequence
  Description of Artificial Sequence Synthetic oligonucleotide
ctgggtcgcttggaagaagccaaggtgccattgaaaatgctgctcttc

SEQ ID NO: 359 - protein, 13 aa, Artificial Sequence
  Description of Artificial Sequence Synthetic peptide
WLLRTWERADSGL

SEQ ID NO: 360 - DNA, 39 bp, Artificial Sequence
  Description of Artificial Sequence Synthetic oligonucleotide
tggctcctgcgcacctgggagagagctgacagtggcctt

SEQ ID NO: 361 - protein, 19 aa, Artificial Sequence
  Description of Artificial Sequence Synthetic peptide
LVARPEIQIFMEEDVMKVE

SEQ ID NO: 362 - DNA, 57 bp, Artificial Sequence
  Description of Artificial Sequence Synthetic oligonucleotide
cttgttgcaaggccagagattcagatctttatggaagaagatgtcatgaaagtagag

SEQ ID NO: 363 - protein, 21 aa, Artificial Sequence
  Description of Artificial Sequence Synthetic peptide
RHQENVHTVLVHGKVKGLFYI

SEQ ID NO: 364 - DNA, 63 bp, Artificial Sequence
  Description of Artificial Sequence Synthetic oligonucleotide
agacaccaggaaaatgtccacactgtattagtacatggaaaagtgaaaggcctgttttatata

SEQ ID NO: 365 - protein, 35 aa, Artificial Sequence
  Description of Artificial Sequence Synthetic polypeptide
IMLIGNKSNCQKQLLRDMKILNEFYHGLILTLQLS

SEQ ID NO: 366 - DNA, 105 bp, Artificial Sequence
  Description of Artificial Sequence Synthetic polynucleotide
atcatgctcattgggaataagagcaactgccagaaacagcttctcagagatatgaagatattgaacgaattttatcatggtctgatattgacattgcaactgtct

SEQ ID NO: 367 - protein, 23 aa, Artificial Sequence
  Description of Artificial Sequence Synthetic peptide
PKVFPKNQVISLIPKAQEDPEHT

SEQ ID NO: 368 - DNA, 69 bp, Artificial Sequence
  Description of Artificial Sequence Synthetic oligonucleotide
ccaaaagtgtttcccaaaaatcaggtaatatctttaattccaaaagcacaggaagaccctgagcatact

SEQ ID NO: 369 - protein, 66 aa, Artificial Sequence
  Description of Artificial Sequence Synthetic polypeptide
GWKICREGIGPVMLTMTSGEWSFSFLEAICGVSREPVPRRRHCIWDTRGGVGLCEASCWRPTSEVG

SEQ ID NO: 370 - DNA, 198 bp, Artificial Sequence
  Description of Artificial Sequence Synthetic polynucleotide
ggctggaagatttgccgggaaggcattgggcctgttatgctcacaatgacttcaggtgaatggagtttcagtttcctggaggccatctgtggagtttccagggaacctgtaccgaggagaaggcattgtatatgggacactagaggaggtgtgggactgtgtgaagccagctgttggaggcctacgagtgaagtggga

SEQ ID NO: 371 - protein, 45 aa, Artificial Sequence
  Description of Artificial Sequence Synthetic polypeptide
IDPSSPLHTYYERITKGRNPERRYMKPERISPGHEAMEKNLEDNL

SEQ ID NO: 372 - DNA, 135 bp, Artificial Sequence
  Description of Artificial Sequence Synthetic polynucleotide
attgatccaagctctcccttacatacctactatgaaagaattactaaaggacgtaatccagaaagaagatatatgaaaccggaacgaatcagtccgggacacgaggccatggaaaaaaaccttgaagataactta

SEQ ID NO: 373 - protein, 47 aa, Artificial Sequence
  Description of Artificial Sequence Synthetic polypeptide
NMADMLYNFLSQTSAAAKLNLQFEMITAVFRLNRCAECLGLLLQENS

SEQ ID NO: 374 - DNA, 141 bp, Artificial Sequence
  Description of Artificial Sequence Synthetic polynucleotide
aacatggctgacatgctttataatttcctttcacagacctcagcagctgccaagttgaatctgcaatttgagatgataacagcagtattcaggctcaatagatgtgcggaatgtctagggctacttctccaggagaacagc

SEQ ID NO: 375 - protein, 36 aa, Artificial Sequence
  Description of Artificial Sequence Synthetic polypeptide
QILQQALKKLPFSLCITECETIAYHLARDSNGNLEL

SEQ ID NO: 376 - DNA, 108 bp, Artificial Sequence
  Description of Artificial Sequence Synthetic polynucleotide
cagatactacagcaagccttgaagaaactgcctttttcattatgtattactgagtgtgagaccatagcctatcaccttgccagggatagtaatggaaacttggaatta

SEQ ID NO: 377 - protein, 27 aa, Artificial Sequence
  Description of Artificial Sequence Synthetic peptide
QILQQALKIAFLKVVVPLKLHPYSFCH

SEQ ID NO: 378 - DNA, 81 bp, Artificial Sequence
  Description of Artificial Sequence Synthetic oligonucleotide
cagatactacagcaagccttgaagatagcatttttgaaagtggtggtgcctttgaaactgcatccttactccttttgtcac

SEQ ID NO: 379 - protein, 98 aa, Artificial Sequence
  Description of Artificial Sequence Synthetic polypeptide
YESPIFSRDRVNPGALSGSLGQARAACWRTLGSLKPGGEFRAAEQRRAGWVPGAPGFTCSLISSRFPRERFCLRHRSSWRGARRVQCVCPGQTEGLWL

SEQ ID NO: 380 - DNA, 294 bp, Artificial Sequence
  Description of Artificial Sequence Synthetic polynucleotide
tatgagagtcccatcttcagcagagaccgagtgaatccaggtgccctgagtggctcgctgggtcaggcccgcgcggcctgctggaggaccttggggtctttgaagcctgggggagagttcagagcggcagaacagcgcagggctggctgggtgccaggcgctcccgggttcacctgcagtttaataagctcgaggtttccgagagagcgcttttgcctacgccatcgcagcagctggcgtggtgcacgccgtgtccaatgcgtgtgccctgggcaaactgaaggcctgtggctg

SEQ ID NO: 381 - protein, 26 aa, Artificial Sequence
  Description of Artificial Sequence Synthetic peptide
FSQNMNTKMIRGTERKQQYYGLQILE

SEQ ID NO: 382 - DNA, 78 bp, Artificial Sequence
  Description of Artificial Sequence Synthetic oligonucleotide
ttttctcagaatatgaatacgaaaatgattagaggaacagaaagaaagcaacagtactatggactacaaattttggaa

SEQ ID NO: 383 - protein, 24 aa, Artificial Sequence
  Description of Artificial Sequence Synthetic peptide
GEPGEDAGIGRPEEREREEEMADQ

SEQ ID NO: 384 - DNA, 72 bp, Artificial Sequence
  Description of Artificial Sequence Synthetic oligonucleotide
ggggagccaggggaggacgcaggaatagggaggcctgaggagagggagagagaggaggagatggctgatcag

SEQ ID NO: 385 - protein, 24 aa, Artificial Sequence
  Description of Artificial Sequence Synthetic peptide
PPGGHSPQDLSIREIFCLGHKEVC

SEQ ID NO: 386 - DNA, 72 bp, Artificial Sequence
  Description of Artificial Sequence Synthetic oligonucleotide
cctccgggtggacattcccctcaggatttgtctattagagagatcttttgccttgggcataaagaagtctgc

SEQ ID NO: 387 - protein, 16 aa, Artificial Sequence
  Description of Artificial Sequence Synthetic peptide
NYRNLVFLDPCFQENL

SEQ ID NO: 388 - DNA, 48 bp, Artificial Sequence
  Description of Artificial Sequence Synthetic oligonucleotide
aactacagaaacctggtcttcctggatccctgttttcaagaaaatctt

SEQ ID NO: 389 - protein, 17 aa, Artificial Sequence
  Description of Artificial Sequence Synthetic peptide
YKLVVVGAAGVGKSALT

SEQ ID NO: 390 - DNA, 51 bp, Artificial Sequence
  Description of Artificial Sequence Synthetic oligonucleotide
tataaacttgtggtagttggagctgctggcgtaggcaagagtgccttgacg

SEQ ID NO: 391 - protein, 17 aa, Artificial Sequence
  Description of Artificial Sequence Synthetic peptide
YKLVVVGADGVGKSALT

SEQ ID NO: 392 - DNA, 51 bp, Artificial Sequence
  Description of Artificial Sequence Synthetic oligonucleotide
tataaacttgtggtagttggagctgatggcgtaggcaagagtgccttgacg

SEQ ID NO: 393 - protein, 17 aa, Artificial Sequence
  Description of Artificial Sequence Synthetic peptide
KLVVVGAGDVGKSALTI

SEQ ID NO: 394 - DNA, 51 bp, Artificial Sequence
  Description of Artificial Sequence Synthetic oligonucleotide
aaacttgtggtagttggagctggtgacgtaggcaagagtgccttgacgata

SEQ ID NO: 395 - protein, 17 aa, Artificial Sequence
  Description of Artificial Sequence Synthetic peptide
KLVVVGAGRVGKSALTI

SEQ ID NO: 396 - DNA, 51 bp, Artificial Sequence
  Description of Artificial Sequence Synthetic oligonucleotide
aaacttgtggtagttggagctggtcgcgtaggcaagagtgccttgacgata

SEQ ID NO: 397 - protein, 17 aa, Artificial Sequence
  Description of Artificial Sequence Synthetic peptide
LDILDTAGHEEYSAMRD

SEQ ID NO: 398 - DNA, 51 bp, Artificial Sequence
  Description of Artificial Sequence Synthetic oligonucleotide
ttggatattctcgacacagcaggtcacgaggagtacagtgcaatgagggac

SEQ ID NO: 399 - protein, 17 aa, Artificial Sequence
  Description of Artificial Sequence Synthetic peptide
LDILDTAGREEYSAMRD

SEQ ID NO: 400 - DNA, 51 bp, Artificial Sequence
  Description of Artificial Sequence Synthetic oligonucleotide
ttggatattctcgacacagcaggtcgagaggagtacagtgcaatgagggac

SEQ ID NO: 401 - protein, 17 aa, Artificial Sequence
  Description of Artificial Sequence Synthetic peptide
LDILDTAGKEEYSAMRD

SEQ ID NO: 402 - DNA, 51 bp, Artificial Sequence
  Description of Artificial Sequence Synthetic oligonucleotide
ttggatattctcgacacagcaggtaaagaggagtacagtgcaatgagggac

SEQ ID NO: 403 - protein, 17 aa, Artificial Sequence
  Description of Artificial Sequence Synthetic peptide
YTHFTSPIKRYADVIVH

SEQ ID NO: 404 - DNA, 51 bp, Artificial Sequence
  Description of Artificial Sequence Synthetic oligonucleotide
tacacacattttacttcacccattaaaagatacgcagatgtcattgttcat

SEQ ID NO: 405 - protein, 17 aa, Artificial Sequence
  Description of Artificial Sequence Synthetic peptide
IGDFGLATEKSRWSGSH

SEQ ID NO: 406 - DNA, 51 bp, Artificial Sequence
  Description of Artificial Sequence Synthetic oligonucleotide
ataggtgattttggtctagctacagagaaatctcgatggagtgggtcccat

SEQ ID NO: 407 - protein, 17 aa, Artificial Sequence
  Description of Artificial Sequence Synthetic peptide
QRIGSGSFATVYKGKWH

SEQ ID NO: 408 - DNA, 51 bp, Artificial Sequence
  Description of Artificial Sequence Synthetic oligonucleotide
caaagaattggatctggatcatttgcaacagtctacaagggaaagtggcat

SEQ ID NO: 409 - protein, 9 aa, Homo sapiens
CTELKLSDY

SEQ ID NO: 410 - protein, 9 aa, Homo sapiens
NLVPMVATV

SEQ ID NO: 411 - protein, 9 aa, Homo sapiens
LIYRRRLMK

SEQ ID NO: 412 - protein, 9 aa, Homo sapiens
LYSACFWWL

SEQ ID NO: 413 - protein, 9 aa, Homo sapiens
NPKASLLSL

SEQ ID NO: 414 - protein, 9 aa, Homo sapiens
ELRSRYWAI

SEQ ID NO: 415 - protein, 9 aa, Homo sapiens
NPKASLLSL

SEQ ID NO: 416 - protein, 9 aa, Homo sapiens
IVTDFSVIK

SEQ ID NO: 417 - protein, 9 aa, Homo sapiens
NPKASLLSL

SEQ ID NO: 418 - protein, 9 aa, Homo sapiens
NLVPMVATV

SEQ ID NO: 419 - protein, 9 aa, Homo sapiens
LIYRRRLMK

SEQ ID NO: 420 - protein, 9 aa, Homo sapiens
NLVPMVATV

SEQ ID NO: 421 - protein, 120 aa, Artificial Sequence
  Description of Artificial Sequence Synthetic polypeptide
TQRLPGHKVSNSCMTGLVLTFTKRDPFHKPVTWCGQLADVRQCMFHSIRERAHPLQRALATLNFFFVSFYTALYLTHPLLTLQTWEVVVTGQESRMFTLLLEISYVSYYCMYLVMRGHVH

SEQ ID NO: 422 - DNA, 360 bp, Artificial Sequence
  Description of Artificial Sequence Synthetic polynucleotide
actcaacggttgccaggacacaaagtaagcaacagctgcatgaccggtttagtcctgacgtttacaaagagggaccctttccataagcctgtaacttggtgtgggcagcttgccgatgtcaggcagtgcatgtttcactcgattagggagagagcgcaccctctccagagggctttggccacgcttaattttttctttgtttccttctatactgctttatatctcacacatcccctcttaactctccagacatgggaagttgttgtgacaggtcaggaaagtcgtatgtttacccttctcctagaaattagttatgtaagctattattgtatgtatttagtaatgaggggacatgtgcat

SEQ ID NO: 423 - protein, 21 aa, Artificial Sequence
  Description of Artificial Sequence Synthetic peptide
MACPGFLWALVISTCLEFSMA

SEQ ID NO: 424 - protein, 77 aa, Artificial Sequence
  Description of Artificial Sequence Synthetic polynucleotide
MQIFVKTLTGKTITLEVEPSDTIENVKAKIQDKEGIPPDQQRLIFAGKQLEDGRTLSDYNIQKESTLHLVLRLRGVR

SEQ ID NO: 425 - protein, 28 aa, Artificial Sequence
  Description of Artificial Sequence Synthetic peptide
MGQKEQIHTLQKNSERMSKQLTRSSQAV